(19) United States
(12) Patent Application Publication
(10) Pub. No.: US 2024/0066133 A1
(43) Pub. Date: Feb. 29, 2024

(54) THERAPEUTIC AGENTS AND CONJUGATES THEREOF

(71) Applicant: Beijing Xuanyi PharmaSciences Co., Ltd., Beijing (CN)

(72) Inventors: Yuntao SONG, Palo Alto, CA (US); Anrong LI, Foster City, CA (US); Hui LI, Santa Clara, CA (US); Xianfeng LI, Cupertino, CA (US); Junbao YANG, San Bruno, CA (US)

(21) Appl. No.: 17/908,812

(22) PCT Filed: Mar. 5, 2021
(86) PCT No.: PCT/US2021/021117

§ 371 Date: Sep. 1, 2022

Related U.S. Application Data
(60) Provisional application No. 62/986,223, filed on Mar. 6, 2020.
(60) Provisional application No. 63/062,503, filed on Aug. 7, 2020.

(51) Int. Cl. A61K 47/55; A61K 47/54; A61K 47/65; A61P 35/00

(52) U.S. Cl. CPC A61K 47/55; A61P 35/00; A61K 47/65; A61K 47/549

(57) ABSTRACT

The present disclosure provides a class of conjugates of general formula (X), a class of TLR9 agonist derivatives, such as formula (I), (XX), and (XXI), certain diastereomers of STING agonists, a class of STING agonist derivatives, such as formula (XXVIV), a class of heterocyclic compounds of general formula (II), a class of heterocyclic compounds of general formula (III), as defined herein. A¹, A², T, Z¹, Z², Z³, b₁, and b₂, in formula (X) are defined herein. The conjugate provides unique properties that are based upon the properties of the therapeutic agents that are part of the conjugate. Also provided are methods of synthesis and use of compounds.

### CROSS-REFERENCE TO RELATED APPLICATIONS

This application claims the benefit of and priority to U.S. Provisional Application No. 62/986,223, filed Mar. 6, 2020, and U.S. Provisional Application No. 63/062,503, filed Aug. 7, 2020, the disclosures of which are hereby incorporated by reference in their entireties for all purposes.

### SEQUENCE LISTING

This application is being filed electronically via EFS-Web and includes an electronically submitted sequence listing in .txt format. The .txt file contains a sequence listing entitled “CSPL_011_02WO_SeqList_ST25.txt” created on Mar. 2, 2021 and having a size of ˜1.87 kilobytes. The sequence listing contained in this .txt file is part of the specification and is incorporated herein by reference in its entirety.

### BACKGROUND

It has long been appreciated by cancer researchers that the phenotypic heterogeneity and progressive evolution of malignant tumors minimize the chance that any agent targeting a single molecular pathway could effectively cure advanced cancer. Indeed, not only does a given cancer cell typically usurp otherwise normal growth and anti-apoptotic mechanisms to avoid dying, it also employs mechanisms to avoid elimination by the immune system and orchestrates changes in the tumor microenvironment (TME) to ensure its survival. (Nature. 2017; 541: 321-30.) Despite recent breakthroughs in immune therapy of cancer, it is appreciated that combination therapies that increase efficacy will be needed to treat patients whose tumors do not respond to current and emerging standards of care.

Activation of the immune system is a key component of mounting an effective durable anti-tumor response. Pathogen-associated molecular patterns (PAMPs) have garnered a great deal of interest as immunotherapy, because they have known mechanisms and can invoke a Th1 immune response. PAMPs consist of one or more structures that are sensed by pattern recognition receptors (PRRs) on host cells and can activate immune responses mediated through one or more signaling pathways. Three common classes of PRRs are Toll-like receptors (TLRs), nucleotide-binding oligomerization domain (NOD)-like receptors (NLRs), and stimulator of interferon genes (STING).

STING Agonists

Detection of pathogen-associated molecular patterns (PAMPs) or danger-associated molecular patterns (DAMPs) by pattern recognition receptors (PRRs) triggers the innate immunity, resulting in production of type I and III IFNs, proinflammatory cytokines and chemokines.

STING (Stimulator of Interferon Genes) is an endoplasmic reticulum (ER) membrane signaling protein, which is involved in innate immune responses to cytosolic nucleic acids, including self- and foreign-derived double stranded DNA and bacterial cyclic dinucleotides.

STING is activated by cyclic dinucleotides such as 2′3′-cGAMP, produced by cGAS in response to cytosolic double-stranded DNA. STING activation induces its relocation from the endoplasmic reticulum to the Golgi. During this process, STING recruits TBK1, which phosphorylates STING, generating a platform for IRF3 recruitment and phosphorylation by TBK1. STING also activates NF-κB. Phosphorylated IRF3 and NF-κB subsequently translocate into the nucleus to induce type I IFN and inflammatory gene expression. In DCs, STING activation additionally induces expression of co-stimulatory molecules, leading to cell maturation and launching of adaptive immunity.

Recent publications suggest that lower dosing of STING agonists is optimal to generate systemic tumor-specific T cell responses and durable antitumor immunity, it could be beneficial to combine low immunogenic dosing regimens of STING agonist with other immunotherapies to achieve good efficacy.

TLR Agonists

Toll-like receptors (TLRs) comprise a family of highly conserved germline-encoded pattern recognition receptors that detect pathogen-associated molecular patterns (PAMPs) expressed by a variety of infectious microorganisms. The ability of TLRs to trigger the innate immune system and bolster adaptive immunity against antigens expressed by pathogens and tumors is well established. At least 13 different TLRs have been identified in mammals, with TLRs 7, 8, and 9 being similar in their recognition of nucleic acid motifs and expression within endosomal compartments.

Studies show that TLR7 is primarily expressed by plasmacytoid dendritic cells (pDC), TLR8 by monocytes, monocyte-derived (m)DCs, macrophages and Langerhans cells, and TLR9 by DCs, B cells, monocytes and mast cells. Synthetic agonist analogs such as imiquimod (R837), resiquimod (R848) and loxoribine are designed to stimulate TLR7 typically trigger TLR8 as well and induce the secretion of IL-12 and TNFα by mDCs and/or pDCs. Many TLR7/8 agonists also enhance the expression of co-stimulatory molecules and the migration of DCs, thereby facilitating the induction of Th1 immune responses. Synthetic oligonucleotides that express CpG motifs (such as PF-3512676, SD-101, CMP-001, MGN-1703, IMO-2125) trigger TLR9 and elicit a Th1-dominated immune response characterized by the production of pro-inflammatory cytokines (including IL-12, IFNα, and TNFα) and the up-regulation of costimulatory (CD80 and CD86) and MHC class I and II molecules. The anti-tumor activity of TLR agonists targeting TLRs 7, 8 and 9 has generally been explored by delivering them systemically or intratumorally to tumors. A growing body of evidence suggests that the efficacy of TLR agonists might be improved by using them in combination.

Codelivery of STING, TLR9 and TLR7/8 Agonists

PAMPs such as STING, TLR9 and TLR7/8 agonists are located on cell membranes or in the cytosol and require intracellular delivery. These cytosolic PRRs predominantly coordinate Th1-biased humoral and cellular immunity. Their activity can be further enhanced through combining two or more PAMPs, particularly those that activate multiple immune signaling pathways. One approach for eliciting broader and more protective cytokine responses is codelivery of PAMPS for concurrent activation of PRRs. (Mol. Pharmaceutics 2018, 15, 11, 4933-4946)

It has been shown experimentally that delivery of PAMPs in the same microparticle (MP) induced superior responses both in vitro and in vivo when compared to the same dose of PAMPs delivered in separate MPs. One likely explanation for this observation is that coencapsulated agonists will always engage their respective PRRs in the same cell (cis-engagement), allowing for PRR cross-talk and enhanced immune activation. Conversely, when PAMPs are delivered in separate particles, both particle types need to be delivered intracellularly in order for PRR crosstalk to occur (trans-engagement). The likelihood of this happening is dependent upon both particle location and the phagocytic capacity of the cell, leading to a more variable response. This cis- vs trans-engagement of receptors is poorly studied in the context of innate immunity but may be important for optimal biological responses, as PRR cross-talk plays a central role in innate immunity and allows the generation of a pathogenspecific immune response. (Int. Rev. Immunol. 2014, 33 (6), 443-53). In addition, it has been shown that the combination of coencapsulated PAMPs had a stronger adjuvant effect than the delivery of individually encapsulated PAMPs. (Nature 2011, 470 (7335), 543-7, Sci. Rep. 2017, 7 (1), 2530.) These works strongly support that coengagement of multiple PRRs is able to generate potent humoral and cellular responses.

Whereas many forms of immunotherapy are effective against tumors, many drugs suffer from unfavorable pharmacokinetic parameters that limit their effectiveness. Rapid clearance of such drugs from physiological compartments, either via metabolism or excretion, results in short lifetimes and reduced exposure to targets. Many therapeutic agonists are attractive immune modulators; however, they are not ideal pharmaceutical agents due to poor pharmacokinetics (PK), poor tolerability, and pleiotropic activity that may be exacerbated by frequent dose administration.

We envision that a drug-drug conjugate with releasable linkers connecting the drugs, would deliver active drugs to activate multiple immune signaling pathways at the same time. The delivery of drugs in this way might activate cis-engagement of receptors and may be important for optimal biological responses. Furthermore, drug-drug conjugate would optimize the pharmacokinetics (PK) properties of the drugs and show advantages over co-administration of each individual drugs.

### SUMMARY OF THE INVENTION

The present disclosure provides in part, a conjugate of formula (X), STING agonists, a compound of formula (III) comprising a releasable linker moiety covalently attached to a therapeutic agent A, a compound of formula (XXII) comprising a linker moiety covalently attached to a STING agonist, a compound of formula (V), (VI), (VII), (VIII), or (IX), a STING agonist derivative of formula (XXVIV), a TLR9 agonist derivative of formula (XX), or (XXI), a TLR9 agonist of formula (I), a releasable linker of formula (II), and compositions and methods thereof.

In one aspect, the present disclosure provides a conjugate of formula (X):

[A²-Z²-T-Z³]_{b2}-A¹-[Z¹-T-Z²-A²]_{b1} (X)

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
b1 is an integer of 0 or 1;
b2 is an integer of 0 or 1;
wherein b1+b2 is 1 or 2;
each T is independently a triazole functional group;
Z¹, Z², and Z³ are each independently a spacer; and
A¹ and A² are each independently a therapeutic agent or an active moiety of a therapeutic agent; or a compound that decomposes to a therapeutic agent; wherein one or more atoms or a chemical group in the therapeutic agent or the compound that decomposes to a therapeutic agent is independently replaced with a covalent bond to the spacer.

In certain embodiments, A¹ and A² are each independently a STING agonist, a TLR9 agonist, or a TLR7/8 agonist.

In certain embodiments, the present disclosure provides a STING agonist represented by:

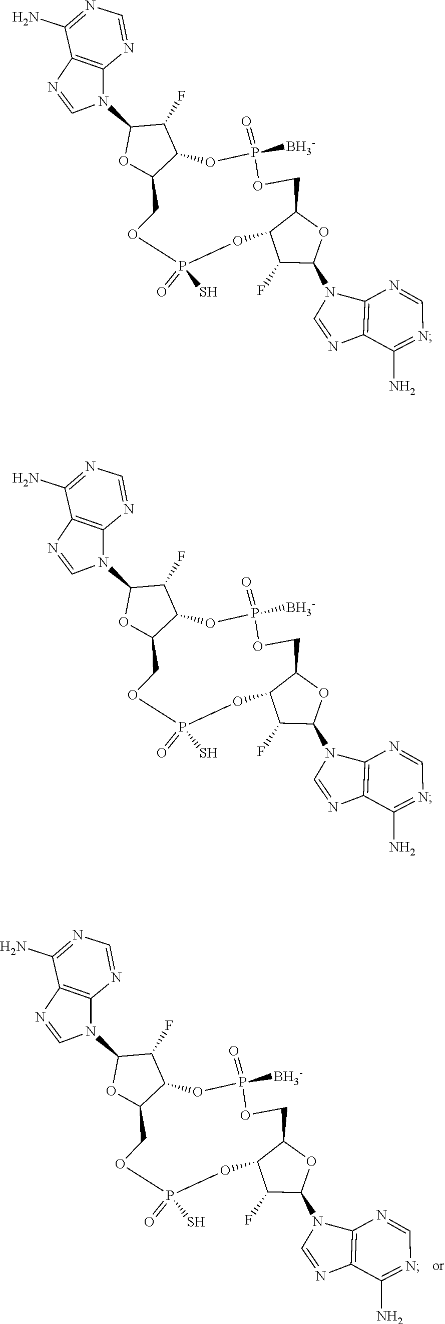

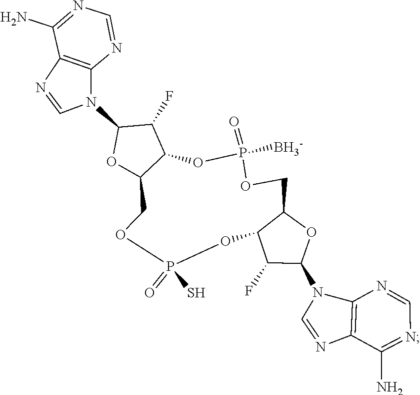

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, the present disclosure relates to conjugates of formula (X), (X-A), (X-B), (X-C), (X-D), (X-E), (X-F), (X-G), (X-H), (XXIV), (XXV), (X-1), (XI), (XI-A), (XI-B), (XXVI), (XXVI-A), (XXVI-B), (XII), (XII-A), (XII-B), (XXVII), (XXVII-A), (XXVII-B), (XIII), (XIII-A), (XIII-B), (XIV), (XIV-A), (XIV-B), (XV), (XV-A), (XV-B), (XVI-1), (XVI), (XVI-A), (XVI-B), (XVII-A), (XVII-B), (XVIII), (XVIII-A), (XVIII-B), (XIX), (XIX-A), (XIX-B), (XXVIII), or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof.

In certain embodiments, the present disclosure provides a compound comprising a releasable linker moiety covalently attached to a therapeutic agent, wherein the compound has a structure according to formula (III):

[Linker]_{b}-A (III);

or an enantiomer, a mixture of enantiomers, a diastereomer, a mixture of two or more diastereomers, a tautomer, a mixture of two or more tautomers, or an isotopic variant thereof;
wherein
A is a therapeutic agent or an active moiety of a therapeutic agent; or a compound that decomposes to a therapeutic agent;
b is an integer of 1 or 2; wherein when b=2, both groups are directly bound to A; and
each linker independently has a structure according to formula (III-L):

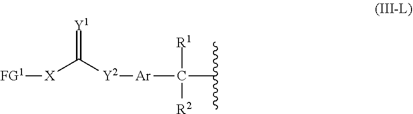

wherein:
Ar is substituted or unsubstituted aryl, or substituted or unsubstituted heteroaryl;
X is a spacer moiety;
each R¹ and R² is, independently, hydrogen, substituted or unsubstituted alkyl, substituted or unsubstituted alkenyl, substituted or unsubstituted alkynyl, or substituted or unsubstituted aryl; or R¹ and R², together with the atom to which they are attached, can join together to form a 3-8 membered ring that can optionally contain one or two heteroatoms;
Y¹ is O or S;
Y² is O, NH or S;
FG¹ is a functional group capable of reacting through click chemistry; and
[symbol] represents a bond to A; wherein one or more atoms in each therapeutic agent or compound that decomposes to a therapeutic agent is independently replaced with a covalent bond to a linker, or a chemical group linking the therapeutic agent or compound to a linker.

In certain embodiments, the present disclosure provides a compound comprising a linker moiety covalently attached to a STING agonist, wherein the compound has a structure according to formula (XXII):

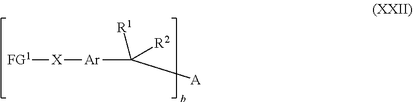

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X is a spacer moiety;
each R¹ and R² is, independently, a hydrogen, substituted or unsubstituted alkyl, substituted or unsubstituted alkenyl, substituted or unsubstituted alkynyl, or substituted or unsubstituted aryl; or
R¹ and R², together with the atom to which they are attached, can join together to form a 3-8 membered ring that can optionally contain one or two heteroatoms;
FG¹ is a functional group capable of reacting through click chemistry;
b is an integer of 1 or 2; wherein when b=2, both groups are directly bound to A; and
A is a STING agonist; wherein one or more atoms in STING agonist is independently replaced with a covalent bond to a linker.

In certain embodiments, the present disclosure provides a compound of formula (VI):

CpG-X-FG¹ (VI)

or an enantiomer, a mixture of enantiomers, a diastereomer, a mixture of two or more diastereomers, a tautomer, a mixture of two or more tautomers, or an isotopic variant thereof;
wherein:
X is a spacer moiety;
FG¹ is a functional group capable of reacting through click chemistry; and
CpG is a TLR9 agonist oligodeoxynucleotide wherein the CpG is covalently bound to X through a 3′-O or 5′-O of a terminal nucleotide of the CpG.

In certain embodiments, the present disclosure provides a compound of formula (V):

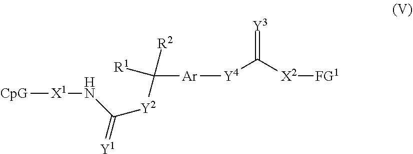

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X¹ and X² are each independently a spacer moiety;
each R¹ and R² is independently, a hydrogen, substituted or unsubstituted alkyl, substituted or unsubstituted alkenyl, substituted or unsubstituted alkynyl, or substituted or unsubstituted aryl; or
R¹ and R², together with the atom to which they are attached, can join together to form a 3-8 membered ring that can optionally contain one or two heteroatoms;
Y¹ is O, NH or S;
Y² is O, NH or S;
Y³ is O, NH or S;
Y⁴ is O, NH or S;
FG¹ is a functional group capable of reacting through click chemistry; and
CpG is a TLR9 agonist oligodeoxynucleotide wherein the CpG is covalently bound to X¹ through a 3′-O or 5′-O of a terminal nucleotide.

In certain embodiments, the present disclosure provides a compound of formula (VII):

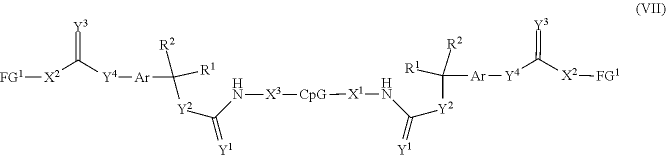

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X¹, X² and X³ are each independently a spacer moiety;
each R¹ and R² is, independently, a hydrogen, substituted or unsubstituted alkyl, substituted or unsubstituted alkenyl, substituted or unsubstituted alkynyl, or substituted or unsubstituted aryl; or
R¹ and R², together with the atom to which they are attached, can join together to form a 3-8 membered ring that can optionally contain one or two heteroatoms;
Y¹ is O, NH, or S;
Y² is O, NH, or S;
Y³ is O, NH, or S;
Y⁴ is O, NH, or S;
FG¹ is a functional group capable of reacting through click chemistry; and
CpG is a TLR9 agonist oligodeoxynucleotide wherein either X¹ is covalently bound to 5′-O of the terminal nucleotide of CpG and X³ is covalently bound to 3′-O of the terminal nucleotide of CpG; or X¹ is covalently bound to 3′-O of the terminal nucleotide of CpG and X³ is covalently bound to 5′-O of the terminal nucleotide of CpG.

In certain embodiments, the present disclosure provides a compound of formula (VIII):

FG¹-X²-CpG-X¹-FG¹ (VIII)

or an enantiomer, a mixture of enantiomers, a diastereomer, a mixture of two or more diastereomers, a tautomer, a mixture of two or more tautomers, or an isotopic variant thereof;
wherein:
X¹ and X² are each independently a spacer moiety;
FG¹ is a functional group capable of reacting through click chemistry; and
CpG is a TLR9 agonist oligodeoxynucleotide wherein either X¹ is covalently bound to 3′-O of the terminal nucleotide of CpG and X² is covalently bound to 5′-O of the terminal nucleotide of CpG; or X¹ is covalently bound to 5′-O of the terminal nucleotide of CpG and X² is covalently bound to 3′-O of the terminal nucleotide of CpG.

In certain embodiments, the present disclosure provides a compound of formula (IX):

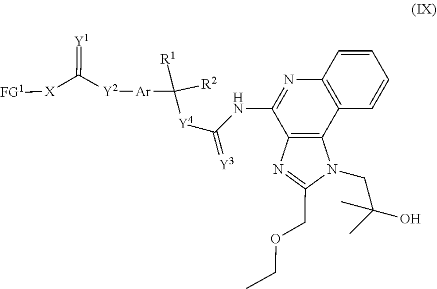

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X is a spacer moiety;
each R¹ and R² is, independently, a hydrogen, substituted or unsubstituted alkyl, substituted or unsubstituted alkenyl, substituted or unsubstituted alkynyl, substituted or unsubstituted aryl; or
R¹ and R², together with the atom to which they are attached, can join together to form a 3-8 membered ring that can optionally contain one or two heteroatoms;
Y¹ is O or S;
Y² is O, NH or S;
Y³ is O or S;
Y⁴ is O or S; and
FG¹ is a functional group capable of reacting through click chemistry.

In certain embodiments, the present disclosure provides a STING agonist derivative that is released from conjugates of the present disclosure, wherein the STING agonist derivative has a structure according to formula (XXVIV):

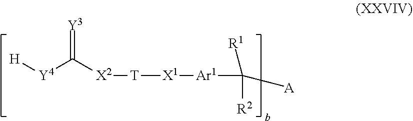

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
Ar¹ is a substituted or unsubstituted aryl, or substituted or unsubstituted heteroaryl;
X¹ and X² are each independently a spacer moiety;
R¹ and R² are each independently a hydrogen, substituted or unsubstituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
Y³ and Y⁴ are each independently O or S;
b is an integer of 1 or 2; wherein when b=2, both groups are directly bound to A;
each T is independently a triazole functional group; and
A is a STING agonist; wherein one or more atoms in the STING agonist is independently replaced with a covalent bond to the spacer.

In certain embodiments, the present disclosure provides a TLR9 agonist derivative that is released from conjugates of the present disclosure, wherein the released TLR9 agonist has a structure according to formula (XX):

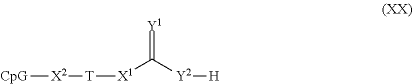

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
X¹ and X² are each independently a spacer moiety;
Y¹ and Y² are each independently O or S;
T is a triazole functional group; and
CpG is a TLR9 agonist oligodeoxynucleotide; wherein one or more atoms in the CpG is independently replaced with a covalent bond to X².

In certain embodiments, the present disclosure provides a TLR9 agonist derivative that is released from conjugates of the present disclosure, wherein the released TLR9 agonist has a structure according to formula (XXI):

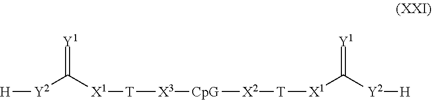

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
X¹, X² and X³ are each independently a spacer moiety;
Y¹ and Y² are each independently O or S;
each T is independently a triazole functional group; and
CpG is a TLR9 agonist oligodeoxynucleotide; wherein one or more atoms in the CpG is independently replaced with a covalent bond to X² and X³.

In certain embodiments, the present disclosure provides a TLR9 agonist having a structure according to formula (I):

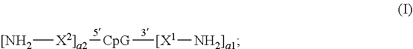

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof;
wherein:
X¹, X² are each independently a spacer moiety;
a1 is an integer of 0 or 1;
a2 is an integer of 0 or 1;
wherein a1+a2 is 1 or 2; and
CpG is a TLR9 agonist oligodeoxynucleotide; wherein one or more atoms in the CpG is independently replaced with a covalent bond to X¹ and/or X².

In certain embodiments, the present disclosure provides a releasable linker having a structure according to formula (II):

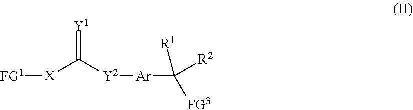

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X is a spacer moiety;
R¹ is hydrogen, substituted or unsubstituted alkyl, substituted or unsubstituted alkenyl, substituted or unsubstituted alkynyl, or substituted or unsubstituted aryl;
R² is hydrogen, substituted or unsubstituted alkyl, substituted or unsubstituted alkenyl, substituted or unsubstituted alkynyl, or substituted or unsubstituted aryl; or
R¹ and R² together with the atom to which they are attached, can join together to form a 3-8 membered ring that can contain one or two heteroatoms;
Y¹ is O, NH or S;
Y² is O, NH or S;
FG¹ is a functional group capable of reacting through click chemistry; and
FG³ is a functional group —OH, SH, LG¹ (leaving group 1) which includes but is not limited to —Cl, —Br, —I,

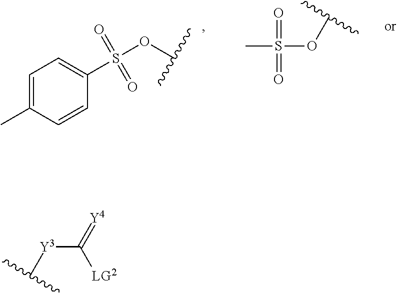

wherein Y³ is O or S; Y⁴ is O or S; LG² is a leaving group

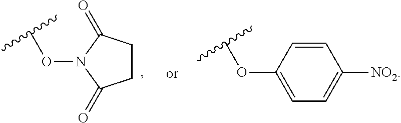 [SMILES: *ON1C(=O)CCC1=O ; *Oc1ccc([N+](=O)[O-])cc1]

In certain embodiments, the present disclosure provides a method for preparing Drug-Drug conjugates (e.g., according to scheme (II)).

In certain embodiments, the present disclosure provides method for the chiral synthesis of the STING agonists, according to Scheme (I).

### BRIEF DESCRIPTION OF THE DRAWINGS

FIG. 1 shows tumor growth curves in a MC38 murine colon cancer model following treatment with compounds A5-I, A5-II, A6-I and A6-II.

FIG. 2 shows tumor growth curves in a MC38 murine colon cancer model following treatment with compounds A1, A11, A1 and A11, and conjugate A16.

FIG. 3 shows tumor growth curves in a MC38 murine colon cancer model following treatment with compounds A11, A12, A13 and A26.

FIG. 4 shows tumor growth curves in a MC38 murine colon cancer model following treatment with compounds A16, A17, A19, A23, A24, A25 and A30.

FIG. 5 shows tumor growth curves in a MC38 murine colon cancer model following treatment with compounds A5-I, A19, A33, A34, A38, A69, A71 and A85.

FIG. 6 shows tumor growth curves in a MC38 murine colon cancer model following treatment with compounds A19, A73, A74 and A75.

FIG. 7 shows tumor growth curves in a MC38 murine colon cancer model following treatment with compound A68.

FIG. 8 shows the structure of formula (XVIII).

FIG. 9 shows the structure of formula (XVIII-A).

FIG. 10 shows the structure of formula (XVIII-B).

FIG. 11 shows the structures of A57, which is a mixture of regioisomers as shown.

FIG. 12 shows the structures of A47, which is a mixture of regioisomers as shown.

FIG. 13 shows the structures of A65, which is a mixture of regioisomers as shown.

FIG. 14 shows the structures of A58, which is a mixture of regioisomers as shown.

FIG. 15 shows the structures of A50, which is a mixture of regioisomers as shown.

FIG. 16 shows the structures of A66, which is a mixture of regioisomers as shown.

### DETAILED DISCLOSURE

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application.

The term “compound(s) of the present invention” or “compound(s) of the present disclosure” refers to compounds of formulae disclosed herein or any subgenera thereof, or a pharmaceutically acceptable salt, stereoisomer, solvate or hydrate thereof, as disclosed herein.

In certain embodiments, intermediates are contemplated as compounds of the present disclosure.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present disclosure contemplates various stereoisomers and mixtures thereof and includes “enantiomers” and a mixture of such isomers is often called an enantiomeric mixture.

“Enantiomers” refer to two stereoisomers of a compound which are non-superimposable mirror images of one another.

A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity. The invention includes all stereoisomers of the compounds described herein. “Diastereomer” refers to a stereoisomer with two or more centers of chirality and whose molecules are not mirror images of one another. Diastereomers have different physical properties, e.g., melting points, boiling points, spectral properties, and reactivities. Mixtures of diastereomers may separate under high resolution analytical procedures such as electrophoresis and chromatography.

The term “regioisomer” is art-recognized and refers to compounds having the same molecular formula but differing in the degree of atomic connectivity. Thus, a “regioselective process” is one in which the formation of a specific regioisomer is advantageous over others, for example, the reaction significantly increases the yield of a specific regioisomer. And so on.

A “tautomer” refers to a proton shift from one atom of a molecule to another atom of the same molecule. The present disclosure includes tautomers of any said compounds.

The terms “pharmaceutical combination,” “therapeutic combination” or “combination” as used herein, refers to a single dosage form comprising at least two therapeutically active agents, or separate dosage forms comprising at least two therapeutically active agents together or separately for use in combination therapy. For example, one therapeutically active agent may be formulated into one dosage form and the other therapeutically active agent may be formulated into a single or different dosage forms. For example, one therapeutically active agent may be formulated into a solid oral dosage form whereas the second therapeutically active agent may be formulated into a solution dosage form for parenteral administration.

The chemical naming protocol and structure diagrams used herein are a modified form of the I.U.P.A.C. nomenclature system, using the ACD/Name Version 9.07 software program, ChemDraw Ultra Version 11.0.1 and/or ChemDraw Ultra Version 14.0 software naming program (Cambridge Soft). For complex chemical names employed herein, a substituent group is named before the group to which it attaches. For example, cyclopropylethyl comprises an ethyl backbone with cyclopropyl substituent. Except as described below, all bonds are identified in the chemical structure diagrams herein, except for some carbon atoms, which are assumed to be bonded to sufficient hydrogen atoms to complete the valency.

The term “composition” or “formulation” denotes one or more substance in a physical form, such as solid, liquid, gas, or a mixture thereof. One example of composition is a pharmaceutical composition, i.e., a composition related to, prepared for, or used in medical treatment.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Salts” include derivatives of an active agent, wherein the active agent is modified by making acid or base addition salts. Preferably, the salts are pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfo aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, PA.

As used herein, “solvate” means a complex formed by solvation (the combination of solvent molecules with molecules or ions of the active agent of the present disclosure), or an aggregate that consists of a solute ion or molecule (the active agent of the present disclosure) with one or more solvent molecules. In the present disclosure, the preferred solvate is hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present disclosure. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The terms “excipient”, “carrier”, and “vehicle” are used interexchangeably throughout this application and denote a substance with which a compound of the present disclosure is administered.

“Therapeutically effective amount” means the amount of a compound or a therapeutically active agent that, when administered to a patient for treating a disease or other undesirable medical condition, is sufficient to have a beneficial effect with respect to that disease or condition. The therapeutically effective amount will vary depending on the type of the selected compound or a therapeutically active agent, the disease or condition and its severity, and the age, weight, etc. of the patient to be treated. Determining the therapeutically effective amount of a given compound or a therapeutically active agent is within the ordinary skill of the art and requires no more than routine experimentation.

“Treating” or “treatment” as used herein covers the treatment of the disease or condition of interest in a mammal, preferably a human, having the disease or condition of interest, and includes: inhibiting the disease or condition, i.e., arresting its development; relieving the disease or condition, i.e., causing regression of the disease or condition; or relieving the symptoms resulting from the disease or condition, i.e., relieving pain without addressing the underlying disease or condition.

As used herein, the terms “disease” and “condition” can be used interchangeably or can be different in that the particular malady or condition cannot have a known causative agent (so that etiology has not yet been worked out) and it is therefore not yet recognized as a disease but only as an undesirable condition or syndrome, wherein a more or less specific set of symptoms have been identified by clinicians.

The present disclosure is also meant to encompass the in vivo metabolic products of the disclosed compounds. Such products can result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabelled compound of the disclosure in a detectable dose to an animal, such as rat, mouse, guinea pig, monkey, or to human, allowing sufficient time for metabolism to occur, and isolating its conversion products from the urine, blood or other biological samples.

As used herein, a “subject” can be a human, non-human primate, mammal, rat, mouse, cow, horse, pig, sheep, goat, dog, cat and the like. The terms “subject” and “patient” are used interchangeably herein in reference, e.g., to a mammalian subject, such as a human subject.

The subject can be suspected of having or at risk for having a cancer, such as prostate cancer, breast cancer, ovarian cancer, salivary gland carcinoma, or endometrial cancer, or suspected of having or at risk for having acne, hirsutism, alopecia, benign prostatic hyperplasia, ovarian cysts, polycystic ovary disease, precocious puberty, spinal and bulbar muscular atrophy, or age-related macular degeneration. Diagnostic methods for various cancers, such as prostate cancer, breast cancer, ovarian cancer, bladder cancer, pancreatic cancer, hepatocellular cancer, salivary gland carcinoma, or endometrial cancer, and diagnostic methods for acne, hirsutism, alopecia, benign prostatic hyperplasia, ovarian cysts, polycystic ovary disease, precocious puberty, spinal and bulbar muscular atrophy, or age-related macular degeneration and the clinical delineation of cancer, such as prostate cancer, breast cancer, ovarian cancer, bladder cancer, pancreatic cancer, hepatocellular cancer, salivary gland carcinoma, or endometrial cancer, diagnoses and the clinical delineation of acne, hirsutism, alopecia, benign prostatic hyperplasia, ovarian cysts, polycystic ovary disease, precocious puberty, spinal and bulbar muscular atrophy, or age-related macular degeneration are known to those of ordinary skill in the art.

“Mammal” includes humans and both domestic animals such as laboratory animals and household pets (e.g., cats, dogs, swine, cattle, sheep, goats, horses, rabbits), and non-domestic animals such as wildlife and the like.

“Optional” or “optionally” means that the subsequently described event of circumstances can or cannot occur, and that the description includes instances where said event or circumstance occurs and instances in which it does not. For example, “optionally substituted aryl” means that the aryl radical can or cannot be substituted and that the description includes both substituted aryl radicals and aryl radicals having no substitution.

The terms below, as used herein, have the following meanings, unless indicated otherwise:

“Acyl” refers to —C(═O)-alkyl radical.

“Amino” refers to the —NH₂ radical.

“Cyano” refers to the —CN radical.

“Halo” “halide” or “halogen” refers to bromo, chloro, fluoro or iodo radical.

“Hydroxy” or “hydroxyl” refers to the —OH radical.

“Imino” refers to the ═NH substituent.

“Nitro” refers to the —NO₂ radical.

“Oxo” refers to the ═O substituent.

“Thioxo” refers to the ═S substituent.

“Sulfhydryl” and “mercapto” refers to —SH radical.

“Alkyl” or “alkyl group” refers to a fully saturated, straight (linear) or branched hydrocarbon chain radical having from one to twenty carbon atoms, and which is attached to the rest of the molecule by a single bond. Alkyls comprising any number of carbon atoms from 1 to 20 are included. An alkyl comprising up to 20 carbon atoms is a C₁-C₂₀ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted. The term “lower alkyl” refers to a C₁-C₆ alkyl, which can be linear or branched, for example including branched C₃-C₆ alkyl.

“Alkylene”, “-alkyl-” or “alkylene chain” refers to a fully saturated, straight or branched divalent hydrocarbon chain radical, and having from one to twenty carbon atoms. Non-limiting examples of C₁-C₂₀ alkylene include methylene, ethylene, propylene, n-butylene, ethenylene, propenylene, n-butenylene, propynylene, n-butynylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twenty carbon atoms, and having one or more carbon-carbon double bonds. Each alkenyl group is attached to the rest of the molecule by a single bond. Alkenyl group comprising any number of carbon atoms from 2 to 20 are included. An alkenyl group comprising up to 20 carbon atoms is a C₂-C₂₀ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₀ alkenyl includes all moieties described above for C₂-C₅ alkenyls and C₂-C₆ alkenyls, but also includes C₇, C₈, C₉ and C₁₀ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkyl group can be optionally substituted.

“Alkenylene” or “alkenylene chain” refers to a straight or branched divalent hydrocarbon chain radical, having from two to twenty carbon atoms, and having one or more carbon-carbon double bonds. Non-limiting examples of C₂-C₂₀ alkenylene include ethene, propene, butene, and the like. The alkenylene chain is attached to the rest of the molecule through a single bond and to the radical group through a single bond. The points of attachment of the alkenylene chain to the rest of the molecule and to the radical group can be through one carbon or any two carbons within the chain. Unless stated otherwise specifically in the specification, an alkenylene chain can be optionally substituted.

“Alkynyl” or “alkynyl group” refers to a straight or branched hydrocarbon chain radical having from two to twenty carbon atoms, and having one or more carbon-carbon triple bonds. Each alkynyl group is attached to the rest of the molecule by a single bond. Alkynyl group comprising any number of carbon atoms from 2 to 20 are included. An alkynyl group comprising up to 20 carbon atoms is a C₂-C₂₀ alkynyl, an alkynyl comprising up to 10 carbon atoms is a C₂-C₁₀ alkynyl, an alkynyl group comprising up to 6 carbon atoms is a C₂-C₆ alkynyl and an alkynyl comprising up to 5 carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all the foregoing moieties, but also includes C₁₁ and C₁₂ alkynyls. Non-limiting examples of C₂-C₁₂ alkenyl include ethynyl, propynyl, butynyl, pentynyl and the like. Unless stated otherwise specifically in the specification, an alkyl group can be optionally substituted.

“Alkynylene” or “alkynylene chain” refers to a straight or branched divalent hydrocarbon chain radical, having from two to twenty carbon atoms, and having one or more carbon-carbon triple bonds. Non-limiting examples of C₂-C₂₀ alkynylene include ethynylene, propargylene and the like. The alkynylene chain is attached to the rest of the molecule through a single bond and to the radical group through a single bond. The points of attachment of the alkynylene chain to the rest of the molecule and to the radical group can be through one carbon or any two carbons within the chain Unless stated otherwise specifically in the specification, an alkynylene chain can be optionally substituted.

“Alkoxy” or “—O-alkyl” refers to a radical of the formula —ORₐ where Rₐ is an alkyl, alkenyl or alkynyl radical as defined above containing one to twenty carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Alkylcarbonyl” refers to the —C(═O)Rₐ moiety, wherein Rₐ is an alkyl, alkenyl or alkynyl radical as defined above. A non-limiting example of an alkyl carbonyl is the methyl carbonyl (“acetal”) moiety. Alkylcarbonyl groups can also be referred to as “Cw-Cz acyl” where w and z depicts the range of the number of carbon in Rₐ, as defined above. For example, “C1-C₁₀ acyl” refers to alkylcarbonyl group as defined above, where Rₐ is C₁-C₁₀ alkyl, C₁-C₁₀ alkenyl, or C₁-C₁₀ alkynyl radical as defined above. Unless stated otherwise specifically in the specification, an alkyl carbonyl group can be optionally substituted.

The term “aminoalkyl” refers to an alkyl group that is substituted with one or more —NH₂ groups. In certain embodiments, an aminoalkyl group is substituted with one, two, three, four, five or more —NH₂ groups. An aminoalkyl group may optionally be substituted with one or more additional substituents as described herein.

“Aryl” refers to a hydrocarbon ring system radical comprising hydrogen, 6 to 18 carbon atoms and at least one aromatic ring. For purposes of this disclosure, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl”, “arylalkyl” or “-alkylaryl” refers to a radical of the formula —R_{b}-R_{c} where R_{b} is an alkylene, alkenylene or alkynylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a rings structure, wherein the atoms which form the ring are each carbon. Carbocyclic rings can comprise from 3 to 20 carbon atoms in the ring. Carbocyclic rings include aryls and cycloalkyl. Cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, preferably having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, bicyclo[3.1.0]hexane, octahydropentalene, bicyclo[1.1.1]pentane, cubane, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, preferably having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, preferably having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Cycloalkylalkyl” or “-alkylcycloalkyl” refers to a radical of the formula —R_{b}-R_{d} where R_{b} is an alkylene, alkenylene, or alkynylene group as defined above and R_{d} is a cycloalkyl, cycloalkenyl, cycloalkynyl radical as defined above. Unless stated otherwise specifically in the specification, a cycloalkylalkyl group can be optionally substituted.

“Haloalkyl” refers to an alkyl radical, as defined above, that is substituted by one, two, three, four, five, six or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one, two, three, four, five, six or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one, two, three, four, five, six or more halo radicals, as defined above, e.g., 1-fluoropropynyl, 1-fluorobutynyl, and the like. Unless stated otherwise specifically in the specification, a haloalkenyl group can be optionally substituted.

“Heterocyclyl,” “heterocyclic ring” or “heterocycle” refers to a stable 3- to 20-membered non-aromatic ring radical which consists of two to twelve carbon atoms and from one to six heteroatoms selected from the group consisting of nitrogen, oxygen and sulfur. Heterocyclyl or heterocyclic rings include heteroaryls as defined below. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

The term “hydroxyalkyl” or “hydroxylalkyl” refers to an alkyl group that is substituted with one or more hydroxyl (—OH) groups. In certain embodiments, a hydroxyalkyl group is substituted with one, two, three, four, five or more —OH groups. A hydroxyalkyl group may optionally be substituted with one or more additional substituents as described herein.

The term “hydrocarbyl” refers to a monovalent hydrocarbon radical, whether aliphatic, partially or fully unsaturated, acyclic, cyclic or aromatic, or any combination of the preceding. In certain embodiments, a hydrocarbyl group has 1 to 40 or more, 1 to 30 or more, 1 to 20 or more, or 1 to 10 or more, carbon atoms. The term “hydrocarbylene” refers to a divalent hydrocarbyl group. A hydrocarbyl or hydrocarbylene group may optionally be substituted with one or more substituents as described herein.

The term “heterohydrocarbyl” refers to a hydrocarbyl group in which one or more of the carbon atoms are each independently replaced by a heteroatom selected from oxygen, sulfur, nitrogen and phosphorus. In certain embodiments, a heterohydrocarbyl group has 1 to or more, 1 to 30 or more, 1 to 20 or more, or 1 to 10 or more, carbon atoms, and 1 to 10 or more, or 1 to 5 or more, heteroatoms. The term “heterohydrocarbylene” refers to a divalent hydrocarbyl group. Examples of heterohydrocarbyl and heterohydrocarbylene groups include without limitation ethylene glycol and polyethylene glycol moieties, such as (—CH₂CH₂O—)ₙH (a monovalent heterohydrocarbyl group) and (—CH₂CH₂O—)ₙ (a divalent heterohydrocarbylene group) where n is an integer from 1 to 12 or more, and propylene glycol and polypropylene glycol moieties, such as (—CH₂CH₂CH₂O—)ₙH and (—CH₂CH(CH₃)O—)ₙH (monovalent heterohydrocarbyl groups) and (—CH₂CH₂CH₂O—)ₙ and (—CH₂CH(CH₃)O—)ₙ (divalent heterohydrocarbylene groups) where n is an integer from 1 to 12 or more. A heterohydrocarbyl or heterohydrocarbylene group may optionally be substituted with one or more substituents as described herein.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heterocyclylalkyl” or “-alkylheterocyclyl” refers to a radical of the formula —R_{b}-Rₑ where R_{b} is an alkylene, alkenylene, or alkynylene chain as defined above and Rₑ is a heterocyclyl radical as defined above, and if the heterocyclyl is a nitrogen-containing heterocyclyl, the heterocyclyl can be attached to the alkyl, alkenyl, alkynyl radical at the nitrogen atom. Unless stated otherwise specifically in the specification, a heterocyclylalkyl group can be optionally substituted.

“Heteroaryl” refers to a 5- to 20-membered ring system radical comprising hydrogen atoms, one to thirteen carbon atoms, one to six heteroatoms selected from the group consisting of nitrogen, oxygen and sulfur, and at least one aromatic ring. For purposes of this disclosure, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” or “-alkylheteroaryl” refers to a radical of the formula —R_{b}-R_{f} where R_{b} is an alkylene, alkenylene, or alkynylene chain as defined above and R_{f} is a heteroaryl radical as defined above. Unless stated otherwise specifically in the specification, a heteroarylalkyl group can be optionally substituted.

“Thioalkyl” refers to a radical of the formula —SRₐ where Rₐ is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (i.e., alkyl, alkylene, alkenyl, alkenylene, alkynyl, alkynylene, alkoxy, alkylamino, alkylcarbonyl, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms with a list provided herein. If no substituent list is included, substituents can be, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with halide, cyano, nitro, hydroxyl, sulfhydryl, amino, —OR_{g}, —SR_{g}, —NRₕRᵢ, alkyl, alkenyl, alkynyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl, heteroaryl, —C(═O)R_{g}, —C(═NRⱼ)R_{g}, —S(═O)R_{g}, —S(═O)₂R_{g}, —S(═O)₂ORₖ, —C(═O)ORₖ, —OC(═O)R_{g}, —C(═O)NRₕRᵢ, —NR_{g}C(═O)R_{g}, —S(═O)₂NRₕRᵢ, —NR_{g}S(═O)₂R_{g}, —OC(═O)OR_{g}, —OC(═O)NRₕRᵢ, —NR_{g}C(═O)OR_{g}, —NR_{g}C(═O)NRₕRᵢ, —NR_{g}C(═NRⱼ)NRₕRᵢ, —P(═O)(R_{g})₂, —P(═O)(ORₖ)R_{g}, —P(═O)(ORₖ)₂, —OP(═O)(R_{g})₂, —OP(═O)(ORₖ)R_{g}, and —OP(═O)(ORₖ)₂, wherein: each occurrence of R_{g} is independently selected from hydrogen, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl; each occurrence of Rₕ and Rᵢ is independently selected from hydrogen, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl, or Rₕ and Rᵢ together with the nitrogen atom to which they are attached, form a heterocyclic or heteroaryl ring; each occurrence of Rⱼ independently is hydrogen, —OR_{g}, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl; and each occurrence of Rₖ independently is hydrogen, W, alkyl, haloalkyl, hydroxyalkyl, aminoalkyl, -alkylcycloalkyl, -alkylheterocyclyl, -alkylaryl, -alkylheteroaryl, cycloalkyl, heterocyclyl, aryl or heteroaryl, wherein each occurrence of W independently is H⁺, Li⁺, Na⁺, K⁺, Cs⁺, Mg⁺², Ca⁺², or —⁺N(R_{g})₂RₕRᵢ.

As used herein, when BH(OR⁷)₂, BH(R^{b})₂, or BH₃ group forms a single bond with a P(═O) group (e.g., the BH(OR⁷)₂, BH(R^{b})₂, or BH₃ has one negative charge.) The “-” in BH(OR⁷)₂⁻, BH(R^{b})₂⁻, and BH₃⁻, indicates that the B group has a single negative charge.

As used herein, the symbol

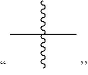

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

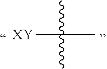

indicates that the chemical entity “XY” is bonded to another chemical entity via the point of attachment bond. Furthermore, the specific point of attachment to the non-depicted chemical entity can be specified by inference. For example, the compound CH₃—R³, wherein R³ is H or

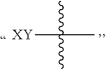

infers that when R³ is “XY”, the point of attachment bond is the same bond as the bond by which R³ is depicted as being bonded to CH₃.

“Fused” refers to any ring structure described herein which is fused to an existing ring structure in the compounds of the disclosure. When the fused ring is a heterocyclyl ring or a heteroaryl ring, any carbon atom on the existing ring structure which becomes part of the fused heterocyclyl ring or the fused heteroaryl ring can be replaced with a nitrogen atom.

“Phosphorothioate bonds” or “phosphorothioate linkage” or “phosphorothioate linked nucleotides” as used herein, occur when a sulfur atom is substituted for a non-bridging oxygen in the phosphate backbone of an oligodeoxynucleotide. As used herein, an asterisk (*) between two nucleotides indicates that these two nucleotides are linked through a phosphorothioate bonds. For example, a sequence of 5′-T*C*G *A-3′ indicates that all of the nucleotides are linked through phosphorothioate bonds, while a sequence of 5′-TC*GA-3′ indicates that C and G are linked through a phosphorothioate bond. As used herein, the phosphorothioate linkage “*” represents both stereoisomer thereof.

The term “immunoregulatory sequence” or “IRS” as used herein refers to a nucleic acid sequence that has immunoregulatory activity as measured in vitro, in vivo and/or ex vivo.

The term “immunoregulatory compound” or “IRC”, as used herein, refers to a molecule, which has immunoregulatory activity and comprises a nucleic acid moiety with an immunoregulatory sequence (IRS). IRCs provided herein contain one or more nucleic acid moieties and one or more non-nucleotide spacer moieties. For example, the IRC may comprise a non-nucleotide spacer bound to a nucleic acid moiety. The IRC can comprise more than one IRS, or at least one IRS. The IRC may comprise a modified and/or unmodified IRS. Modified IRS can include modifications to the Sugar, base or backbone. Accordingly, the term IRC includes compounds, which incorporate one or more nucleic acid moieties covalently bound to a non-nucleotide spacer moiety, wherein at least one of the nucleic acid moieties comprises an IRS. In some embodiments, the non-nucleotide spacer is covalently bound to the nucleic acid moiety through the 3′-O or 5′-O of a terminal nucleotide. In an IRC comprising more than one spacer moiety, the spacers may be the same or different.

The term “nucleic acid moiety” as used herein, refers to a nucleotide monomer (i.e., a mononucleotide) or polymer (i.e., comprising at least 2 contiguous nucleotides). As used herein, a nucleotide comprises (1) a purine or pyrimidine base linked to a sugar that is in an ester linkage to a phosphate group, or (2) an analog in which the base and/or sugar and/or phosphate ester are replaced by analogs, e.g., as described herein. In an IRC comprising more than one nucleic acid moiety, the nucleic acid moieties may be the same or different. Accordingly, in various variations, IRCs incorporated into the immunoregulatory compositions comprise (a) nucleic acid moieties with the same sequence, (b) more than one iteration of a nucleic acid moiety, or (c) two or more different nucleic acid moieties. Additionally, a single nucleic acid moiety may comprise more than one IRS, which may be adjacent, overlapping, or separated by additional nucleotide bases within the nucleic acid moiety.

Nucleic acid moieties used in IRCs incorporated in the immunoregulatory compositions may comprise any of the IRS sequences disclosed herein, and may additionally be sequences of six base pairs or less. It is contemplated that in an IRC comprising multiple nucleic acid moieties, the nucleic acid moieties can be the same or different lengths. In some variations where the IRC comprises more than one nucleic acid moiety, only one of the moieties need comprise the IRS. In some variations, the IRS is a modified IRS. In some variations, the IRS is an unmodified IRS.

The term “immunoregulatory polynucleotide” or “IRP” as used herein refers to a polynucleotide comprising at least one IRS that has immunoregulatory activity as measured in vitro, in vivo and/or ex vivo.

The term diane whipple37 conjugate’ refers to a compound in which two or more the same or different therapeutic agents are linked together by one or more the same or different linker moieties. The therapeutic agent can be an IRP and/or an IRC. Such conjugate linker moiety can be metabolically or chemically stable or unstable.

As used herein, the notation 3′ generally refers to a region or position in a polynucleotide or oligonucleotide that is 3′ (downstream) from another region or position in the same polynucleotide or oligonucleotide. The term “3′ end” refers to the 3′ terminus of the polynucleotide.

As used herein, the notation 5′ generally refers to a region or position in a polynucleotide or oligonucleotide that is 5′ (upstream) from another region or position in the same polynucleotide or oligonucleotide. The term “5′ end” refers to the 5′ terminus of the polynucleotide.

The term “oligonucleotide” or “polynucleotide” as used herein refers to a nucleic acid sequence comprising 2 or more nucleotides, generally at least about 6 nucleotides to about 100,000 nucleotides, or about 6 to about 2000 nucleotides, or about 6 to about 300 nucleotides, or about 20 to about 300 nucleotides, or about 20 to about 100 nucleotides. The terms “oligonucleotide” or “oligomer” also refer to a nucleic acid sequence comprising more than 100 to about 2000 nucleotides, or more than 100 to about 1000 nucleotides, or more than 100 to about 500 nucleotides. The term “Oligonucleotide” also generally refers to any polyribonucleotide or polydeoxyribonucleotide, which may be unmodified RNA or DNA or modified RNA or DNA.

“Oligonucleotides” include without limitation single stranded DNA (ssDNA), double-stranded DNA (dsDNA), single-stranded RNA (ssRNA) and double-stranded RNA (dsRNA), modified polynucleotides and polynucleosides or combinations thereof. The polynucleotide can be linearly or circularly configured, or the polynucleotide can contain both linear and circular segments. Polynucleotides are polymers of nucleosides joined, generally, through phosphodiester linkages, although alternate linkages, such as phosphorothioate esters may also be used in polynucleotides. A nucleoside consists of a purine (adenine (A) or guanine (G) or derivative thereof) or pyrimidine (thymine (T), cytosine (C) or uracil (U), or derivative thereof) base bonded to a sugar. The four nucleoside units (or bases) in DNA are called deoxyadenosine, deoxyguanosine, deoxythymidine, and deoxycytidine. A nucleotide is a phosphate ester of a nucleoside.

The term “oligodeoxynucleotide” as used herein is an oligonucleotide whose nucleotides contain deoxyribose.

The term 5′-O as used herein refers to the oxygen attached to the 5′ carbon of a deoxyribonucleotide or a ribonucleotide. The term 3′-O as used herein refers to the oxygen attached to the 3′ carbon of a deoxyribonucleotide or ribonucleotide. For example, as shown below the 5′-O of a deoxyribonucleotide is the oxygen attached to the 5′ carbon of the deoxyribonucleotide, and the 3′-O is the oxygen attached to the 3′ carbon of the deoxyribonucleotide:

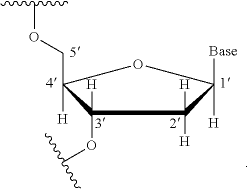

“Immunostimulatory nucleic acid” As used herein, the term immunostimulatory nucleic acid refers to a nucleic acid capable of inducing and/or enhancing an immune response. Immunostimulatory nucleic acids, as used herein, comprise ribonucleic acids and in particular deoxyribonucleic acids. Preferably, immunostimulatory nucleic acids contain at least one CpG motif e.g. a CG dinucleotide in which the C is unmethylated. The CG dinucleotide can be part of a palindromic sequence or can be encompassed within a non-palindromic sequence. The term “immunostimulatory nucleic acid” as used herein should also refer to nucleic acids that contain modified bases such as 4-bromo-cytosine.

The term “CpG oligodeoxynucleotides” (or CpG ODN) as used herein are short single-stranded synthetic DNA molecules that contain a cytosine phosphate deoxynucleotide (“C”) followed by a guanine phosphate deoxynucleotide (“G”). The “p” refers to the phosphodiester link between consecutive nucleotides, although some ODN have a modified phosphorothioate (PS) backbone instead. In certain embodiments, one or more of the internucleotide linkages of the CpG ODN are modified linkages. In some embodiments, one or more of the internucleotide linkages of CpG ODN are phosphorothioate (PS) linkages. In some embodiments, all of the internucleotide linkages of CpG-ODN are phosphorothioate (PS) linkages. A phosphorothioate backbone refers to all of the internucleotide linkages of CpG ODN being phosphorothioate (PS) linkages. Three major classes of stimulatory CpG ODNs have been identified based on structural characteristics and activity on human peripheral blood mononuclear cells (PBMCs), in particular B cells and plasmacytoid dendritic cells (pDCs). These three classes are Class A (Type D), Class B (Type K) and Class C. Synthetic oligodeoxynucleotides (ODN) containing unmethylated CpG motifs (CpG-ODN) act as potent immune stimulators. CpGs provided herein can stimulate/activate, e.g., have a mitogenic effect on, or induce and/or increase cytokine expression by, a vertebrate bone marrow derived cell. For example, CpGs can be useful in activating B cells, NK cells, and antigen-presenting cells, such as monocytes, dendritic cells and macrophages, and T cells. The CpGs can include nucleotide modifications/analogs such as phosphorothioate modifications and can be double-stranded or single-stranded. Generally, double-stranded molecules are more stable in vivo, while single-stranded molecules have increased immune activity.

As used herein, the term “functional group” or any synonym thereof is meant to encompass protected forms thereof as well as unprotected forms.

As used herein, the term “electron altering group” is meant to include any atom or functional group that modifies the electron density of the moiety to which it is attached. Electron altering groups include electron donating groups, which donate electron density (e.g., amine, hydroxy, alkoxyl, alkyl) and electron withdrawing groups (e.g., nitro, cyano, trifluoromethyl) which withdraw electron density.

The terms “spacer”, “spacer moiety”, “non-nucleotide spacer”, “non-nucleotide spacer moiety”, “linkage” and “linker” are used herein to refer to a bond or an atom or a collection of atoms optionally used to link interconnecting moieties such as a terminus of a macromolecule segment (e.g., the 5′-terminus of a CpG oligodeoxynucleotide) and a second segment (e.g., a cyclic dinucleotide, and a protein or an electrophile or nucleophile of a protein). The spacer moiety may be hydrolytically stable or may include a physiologically hydrolyzable or enzymatically degradable linkage. Unless the context clearly dictates otherwise, a spacer moiety optionally exists between any two elements of a compound (e.g., the provided conjugates comprising a CpG moiety and a cyclic dinucleotide moiety, which are attached directly or indirectly through a spacer moiety).

Suitable spacers of the present disclosure, include spacers comprising a linker that can include one or more of carbon atoms, nitrogen atoms, sulfur atoms, phosphorus atoms, oxygen atoms, and combinations thereof. A suitable spacer moiety may comprise an amide, secondary amine, carbamate, thioether, phosphate, phosphorothioate, disulfide group and/or click chemistry product groups. Non-limiting examples of specific spacer moieties include those selected from the group consisting of —O—, —S—, —S—S—, —C(O)—, —C(O)—NH—, —NH—C(O)—NH—, —O—C(O)—NH—, —OP(O)(OH)—, —OP(S)(OH)—, —C(S)—, —CH₂—, —CH₂—CH₂—, —CH₂—CH₂—CH₂—, —CH₂—CH₂—CH₂—CH₂—, —CH₂—CH₂—CH₂—CH₂—CH₂—, O—CH₂—, —CH₂—O—, —O—CH₂—CH₂—, —CH₂—O—CH₂—, —CH₂—CH₂—O—, —O—CH₂—CH₂—CH₂—, —CH₂—O—CH₂—CH₂—, —CH₂—CH₂—O—CH₂—, —CH₂—CH₂—CH₂—O—, —O—CH₂—CH₂—CH₂—CH₂—, —CH₂—O—CH₂—CH₂—CH₂—, —CH₂—CH₂—O—CH₂—CH₂—, —CH₂—CH₂—CH₂—O—CH₂—, —CH₂—CH₂—CH₂—CH₂—O—, —C(O)—NH—CH₂—, —C(O)—NH—CH₂—CH₂—, —CH₂—C(O)—NH—CH₂—, —CH₂—CH₂—C(O)—NH—, —C(O)—NH—CH₂—CH₂—CH₂—, —CH₂—C(O)—NH—CH₂—CH₂—, —CH₂—CH₂—C(O)—NH—CH₂—, —CH₂—CH₂—CH₂—C(O)—NH—, —C(O)—NH—CH₂—CH₂—CH₂—CH₂—, —CH₂—C(O)—NH—CH₂—CH₂—CH₂—, —CH₂—CH₂—C(O)—NH—CH₂—CH₂—, —CH₂—CH₂—CH₂—C(O)—NH—CH₂—, —CH₂—CH₂—CH₂—C(O)—NH—CH₂—CH₂—, —CH₂—CH₂—CH₂—CH₂—C(O)—NH—, —C(O)—O—CH₂—, —CH₂—C(O)—O—CH₂—, —CH₂—CH₂—C(O)—O—CH₂—, —C(O)—O—CH₂—CH₂—, —NH—C(O)—CH₂—, —CH₂—NH—C(O)—CH₂—, —CH₂—CH₂—NH—C(O)—CH₂—, —NH—C(O)—CH₂—CH₂—, —CH₂—NH—C(O)—CH₂—CH₂—, —CH₂—CH₂—NH—C(O)—CH₂—CH₂—, —C(O)—NH—CH₂—, —C(O)—NH—CH₂—CH₂—, —O—C(O)—NH—CH₂—, —O—C(O)—NH—CH₂—CH₂—, —NH—CH₂—, —NH—CH₂—CH₂—, —CH₂—NH—CH₂—, —CH₂—CH₂—NH—CH₂—, —C(O)—CH₂—, —C(O)—CH₂—CH₂—, —CH₂—C(O)—CH₂—, —CH₂—CH₂—C(O)—CH₂—, —CH₂—CH₂—C(O)—CH₂—CH₂—, —CH₂—CH₂—C(O)—, —CH₂—CH₂—CH₂—C(O)—NH—CH₂—CH₂—NH—, —CH₂—CH₂—CH₂—C(O)—NH—CH₂—CH₂—NH—C(O)—, —CH₂—CH₂—CH₂—C(O)—NH—CH₂—CH₂—NH—C(O)—CH₂—, —CH₂—CH₂—CH₂—C(O)—NH—CH₂—CH₂—NH—C(O)—CH₂—CH₂—, —O—C(O)—NH—[CH₂]ₗ—(OCH₂CH₂)ₘ—, bivalent cycloalkyl group, bivalent aryl, —O—, —S—, a divalent amino acid residue, —N(R³)—, and combinations of two or more of any of the foregoing, wherein R³ is H or an organic radical selected from the groups consisting of substituted or unsubstituted alkyl, substituted or unsubstituted alkenyl, substituted or unsubstituted alkynyl, substituted or unsubstituted aryl, (1) is zero to six, and (m) is zero to 20. Other specific spacer moieties have the following structures: —C(O)—NH—(CH₂)₁₋₆—NH—C(O)—, —NH—C(O)—NH—(CH₂)₁₋₆—NH—C(O)—, and —O—C(O)—NH—(CH₂)₁₋₆—NH—C(O)—, wherein the subscript values following each methylene indicate the number of methylenes contained in the structure, e.g., (CH₂)₁₋₆ means that the structure can contain 1, 2, 3, 4, 5 or 6 methylenes.

An “organic radical” as used herein shall include alkyl, substituted alkyl, aryl, and substituted aryl.

A “physiologically cleavable” or “hydrolyzable” or “degradable” bond is a bond that reacts with water (i.e., is hydrolyzed) under physiological conditions. The tendency of a bond to hydrolyze in water will depend not only on the general type of linkage connecting two central atoms but also on the substituents attached to these central atoms. Appropriate hydrolytically unstable or weak linkages include but are not limited to carbamate, carboxylate ester, phosphate ester, anhydrides, acetals, ketals, acyloxyalkyl ether, imines, orthoesters, peptides and oligonucleotides.

A “releasable linker” refers to a linker that connects different therapeutic agents in the conjugates. Either through hydrolysis, enzymatic processes, catalytic processes or otherwise, the therapeutic agent is released, thereby resulting in the unconjugated moiety. In certain embodiments, the releasable linker releases the therapeutic agent by the aforementioned processes that take place in vivo.

“Optional” or “optionally” means that the subsequently described circumstance may or may not occur, so that the description includes instances where the circumstance occurs and instances where it does not.

The present disclosure includes all pharmaceutically acceptable isotopically labeled compounds of the disclosure wherein one or more atoms are replaced by atoms having the same atomic number, but an atomic mass or mass number different from the atomic mass or mass number usually found in nature. Examples of isotopes suitable for inclusion in the compounds of the disclosure include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S.

Certain isotopically-labeled compounds of the disclosure, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of the disclosure can generally be prepared by conventional techniques known to those skilled in the art.

The phrase “an enantiomer, a mixture of enantiomers, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof” has the same meaning as the phrase “(i) an enantiomer, a mixture of enantiomers, a mixture of two or more diastereomers, a tautomer, a mixture of two or more tautomers, a regioisomer, a mixture of two or more regioisomers, or an isotopic variant of the compound referenced therein; (ii) a pharmaceutically acceptable salt, solvate, hydrate, or prodrug of the compound referenced therein; or (iii) a pharmaceutically acceptable salt, solvate, hydrate, or prodrug of an enantiomer, a mixture of enantiomers, a mixture of two or more diastereomers, a tautomer, a mixture of two or more tautomers, a regioisomer, a mixture of two or more regioisomers, or an isotopic variant of the compound referenced therein.”

In certain embodiments, the present disclosure provides a STING agonist selected from the group consisting of:

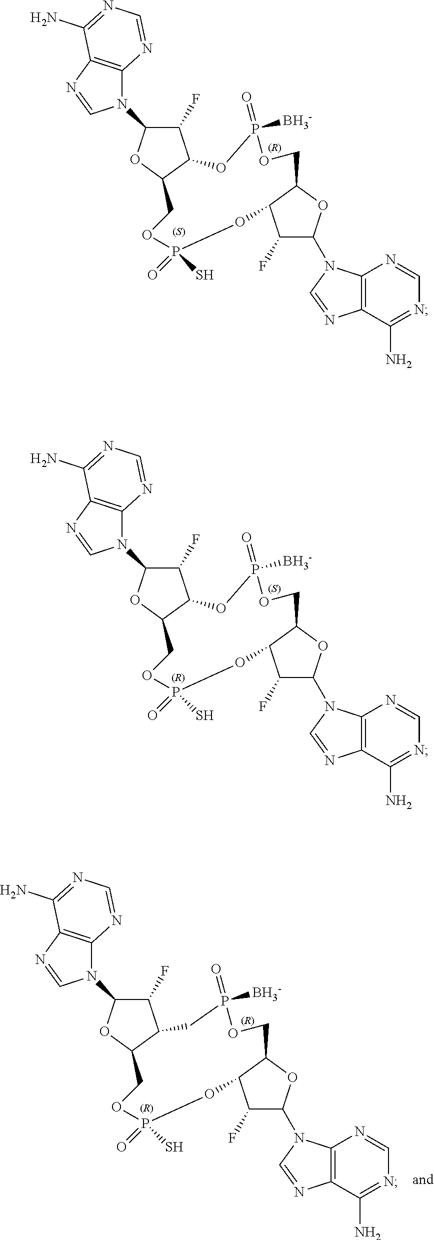

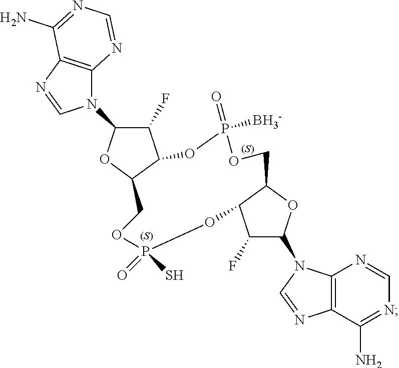

or a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof.

In certain embodiments, provided herein is a method for the synthesis of optically pure STING agonists as shown in Scheme 1.

In certain embodiments, the present disclosure provides for large scale quantities optically pure STING agonists. In certain embodiments, the STING agonist is a STING agonist disclosed in WO 2019/043634, the disclosure of which is hereby incorporated by reference in its entirety for all purposes.

In one aspect, the present disclosure provides a TLR9 agonist of formula (I):

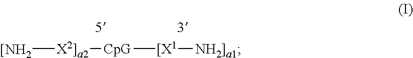

or an enantiomer, a mixture of enantiomers, a diastereomer, a mixture of two or more diastereomers, a tautomer, a mixture of two or more tautomers, or an isotopic variant thereof; or a pharmaceutically acceptable salt, solvate, hydrate, or prodrug thereof;
wherein:
X¹ and X² are each independently a spacer moiety; wherein X¹ is connected to the 3′-terminal nucleotide of the CpG, and X² is connected to the 5′-terminal nucleotide of the CpG moiety;
a1 is an integer of 0 or 1;
a2 is an integer of 0 or 1;
with the proviso that a1+a2 is 1 or 2; and
CpG is an TLR9 agonist oligodeoxynucleotide moiety; wherein one or more atoms in the CpG is independently replaced with a covalent bond to X¹ and/or X².

In certain embodiments of the TLR9 agonist of formula (I), the CpG is an oligodeoxynucleotide moiety that comprises a cytosine deoxynucleotide (“C”) followed by a guanine deoxynucleotide (“G”). In some embodiments there is at least one phosphorothioate link between two nucleotides in the CpG.

In certain embodiments of the TLR9 agonist of formula (I), the CpG is a TLR9 agonist ODNs (oligodeoxynucleotides). In certain embodiments, the ODN is a short synthetic single-stranded DNA molecules containing unmethylated CpG motifs.

In certain embodiments, wherein CpG comprises a following formula:

5′X₁X₂CGX₃X₄3′
wherein X₁, X₂, X₃, and X₄ are any nucleotide, and
CpG is connecting with spacer at 5′-O or/and 3′-O of the terminal nucleotide.

In certain embodiments, CpG comprises a following formula:

5′N₁X₁CGX₂N₂3′
wherein at least one nucleotide separates consecutive CpGs; X₁ is adenine, guanine, or thymine; X₂ is cytosine or thymine; N is any nucleotide and N₁+N₂ is from about 0-26 bases with the proviso that N₁ and N₂ does not contain a CCGG quadmer or more than one CCG or CGG trimer; the nucleic acid sequence is from about 8-30 bases in length; and
CpG is connecting with spacer at 5′-O or/and 3′-O of the terminal nucleotide.

In certain embodiments, CpG comprises the following formula:

5′-Nₓ(TCG(N_{q}))_{y}N_{w}(X₁X₂CGX₂′X₁′(CG)ₚ)_{z} a)
wherein N are nucleosides, x=0, y=1, w=0, p=0 or 1, q=0, 1 or 2, and z=1-20, X₁ and X₁′ are self-complimentary nucleosides, X₂ and X₂′ are self-complimentary nucleosides, and wherein the 5′ T of the (TCG(N_{q}))_{y} sequence is positioned at the 5′ end of the polynucleotide; and
b) a palindromic sequence at least 8 bases in length wherein the palindromic sequence comprises the first (X₁X₂CGX₂′X₁′) of the (X₁X₂CGX₂′X₁′(CG)ₚ)_{z} sequences, wherein the polynucleotide is at least 15 bases in length; and
CpG is connecting with spacer at 5′-O or/and 3′-O of the terminal nucleotide.

In certain embodiments, CpG comprises at least two oligonucleotides linked together at their 3′ ends, an internucleotide linkage, or a functionalized nucleobase or sugar by a non-nucleotidic linker;
wherein at least one of the oligonucleotides is an immunostimulatory oligonucleotide having an accessible 5′ end and comprising an immunostimulatory dinucleotide selected from the group consisting of CG, C^{#}G, CG^{#}, and C^{#}G^{#};
wherein C is cytidine or 2′-deoxycytidine, C^{#} is 2′-deoxythymidine, arabinocytidine, 2′-deoxy-2′-substituted arabinocytidine, 2′-O-substituted arabinocytidine, 2′-deoxy-5-hydroxycytidine, 2′-deoxy-N4-alkyl-cytidine, 2′-deoxy-4-thiouridine or other non-natural pyrimidine nucleoside, G is guanosine or 2′-deoxyguanosine, G^{#} is 2′-deoxy-7-deazaguanosine, 2′-deoxy-6-thioguanosine, arabinoguanosine, 2′-deoxy-2′ substituted-arabinoguanosine, 2′-O-substituted-arabinoguanosine, or other non-natural purine nucleoside;
p is an internucleotide linkage selected from the group consisting of phosphodiester, phosphorothioate, and phosphorodithioate; and
CpG is connecting with spacer at one or two 5′-O of the terminal nucleotide or/and internucleotide linkage.

In certain embodiments of the TLR9 agonist of formula (I), the CpG is a phosphorothioate linked 5′-T*C*G *A*A*C *G*T*T *C*G*A *A*C*G *T*T*C *G*A*A *C*G*T *T*C*G *A*A*T-3′ (SD-101; SEQ ID NO: 1), connecting at 5′-O or/and 3′-O of the terminal nucleotide; a phosphorothioate linked 5′-T*C*G₁*A*A*C*G₁*T*T*C*G₁*-X-*G₁*C*T*T*G₁*C*A*A*G₁*C*T*-5′, wherein X is a glycerol linker and GI is 2′-deoxy-7-deazaguanosine (IMO-2125; SEQ ID NO: 2), connecting at one or two 5′-O of the terminal nucleotide or/and glycerol; 5′-GGGGGGGGGGGACGATCGTCGGGGGGGGGG-3′ (CMP-001; SEQ ID NO: 3), connecting at 5′-O or/and 3′-O of the terminal nucleotide; or phosphorothioate linked 5′-T*C*G*T*C*G*T*T*T*T*G*T*C*G*T*T*T*T*G*T*C*G*T*T-3′ (PF-3512676; SEQ ID NO: 4), connecting at 5′-O or/and 3′-O of the terminal nucleotide.

TABLE A

| SEQ | |
|---|---|
| ID | |
| NO: | Sequence |
| 1 | 5′-T*C*G *A*A*C *G*T*T *C*G*A *A*C*G *T*T*C |
| | *G*A*A *C*G*T *T*C*G *A*A*T - 3′ |
| 2 | 5′-T*C*G₁*A*A*C*G₁*T*T*C*G₁*- X- |
| | *G₁*C*T*T*G₁*C*A*A*G₁*C*T *- 5′ |
| | wherein X is a glycerol linker and G₁ is |
| | 2′-deoxy-7-deazaguanosine |
| 3 | 5′-GGGGGGGGGGGACGATCGTCGGGGGGGGGG-3′ |
| 4 | 5′-T*C*G*T*C*G*T*T*T*T*G*T*C*G*T*T*T*T*G*T* |
| | C*G*T*T-3′ |

In certain embodiments of the TLR9 agonist of formula (I), X¹ and X² are each independently: C₃-C₁₂ alkyl;

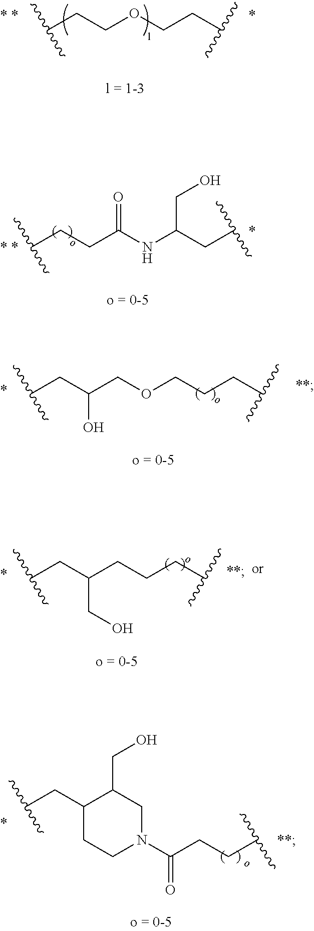

wherein * indicates the attachment point connecting to CpG and ** indicates the attachment point connecting to —NH₂.

In certain embodiments of the TLR9 agonist of formula (I), X¹ and X² are each independently: **—C₃-C₁₂ alkylene-L¹-*;

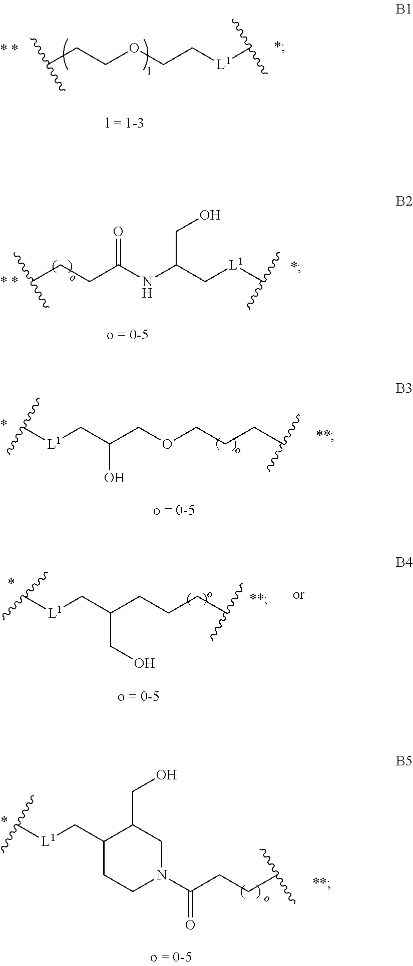

wherein L¹ is independently a bond, —OP(O)(OH)—, or —OP(S)(OH)—; * indicates the attachment point connecting to a 3′-O or 5′-O of a terminal nucleotide of the CpG and ** indicates the attachment point connecting to —NH₂.

In certain embodiments of the TLR9 agonist of formula (I), X² is **—C₃-C₁₂ alkylene-L¹-*; wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; and X¹ is selected from spacer B3 as described herein, wherein L¹ is independently —OP(O)(OH)—, or —OP(S)(OH)—; * indicates the attachment point connecting to a 3′-O of a terminal nucleotide of the CpG; ** indicates the attachment point connecting to the amino group. In certain embodiments, CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at 5′-O or/and 3′-O of the terminal nucleotide.

In certain embodiments of the TLR9 agonist of formula (I), the TLR9 agonist is:

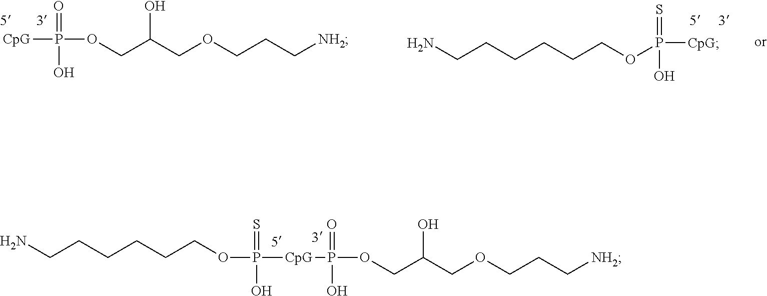

wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at 5′-O or/and 3′-O of the terminal nucleotide.

In certain embodiments, the present disclosure provides a releasable linker of formula (II):

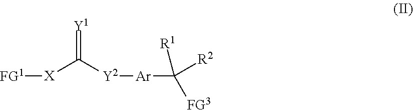

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X is a spacer moiety;
R¹ and R² are, independently, a hydrogen, substituted or unsubstituted alkyl, substituted or unsubstituted alkenyl, substituted or unsubstituted alkynyl, aryl, or substituted aryl, or R¹ and R² together with the atom to which they are attached form a 3-8 membered ring that can contain one or two heteroatoms;
Y¹ is O, NH or S;
Y² is O, NH or S;
FG¹ is a functional group capable of reacting through click chemistry; and
FG³ is a functional group selected from —OH, SH, LG¹ (leaving group 1) which includes but is not limited to —Cl, —Br, —I,

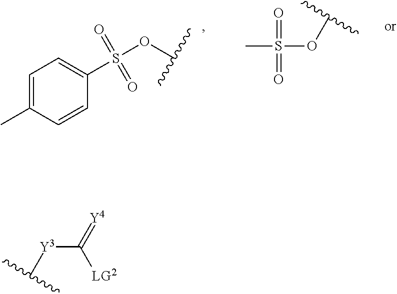

wherein Y³ is O or S; Y⁴ is O or S; and LG² is a leaving group that includes but is not limited to

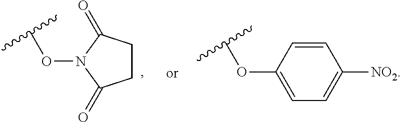 [SMILES: *ON1C(=O)CCC1=O ; *Oc1ccc([N+](=O)[O-])cc1]

In certain embodiments, FG³ is a group selected from —OH, —Cl, —Br, —I,

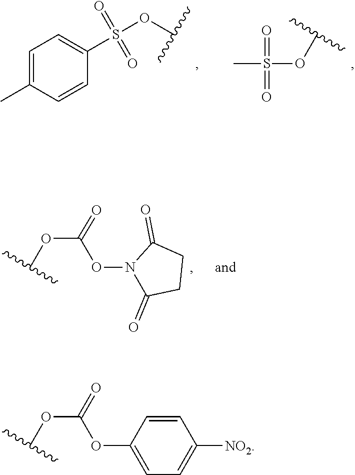

In certain embodiments of the releasable linker of formula (II), the releasable linker has a structure according to formula (II-A):

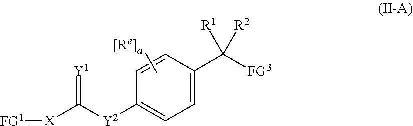

wherein:
X is:

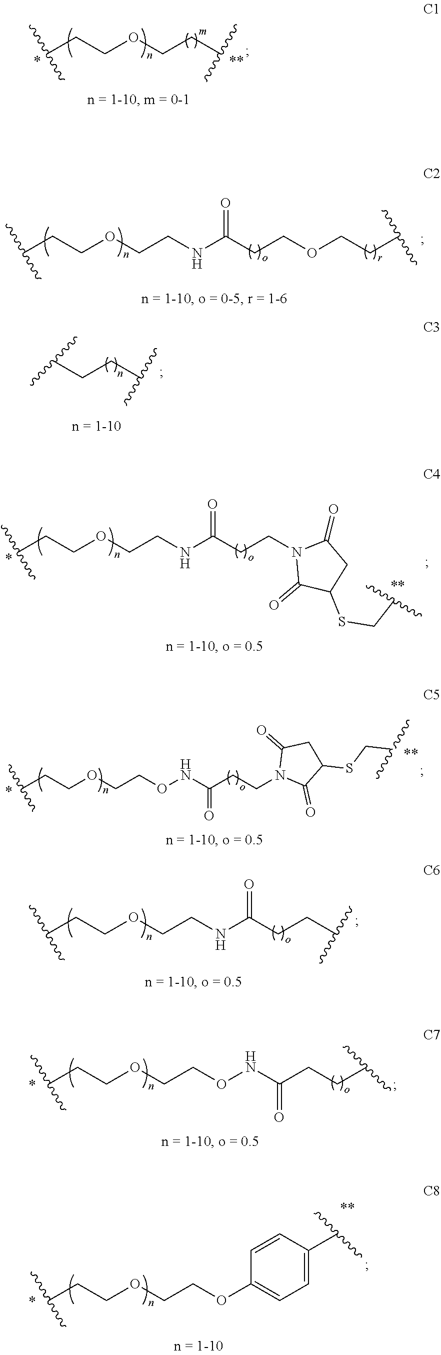

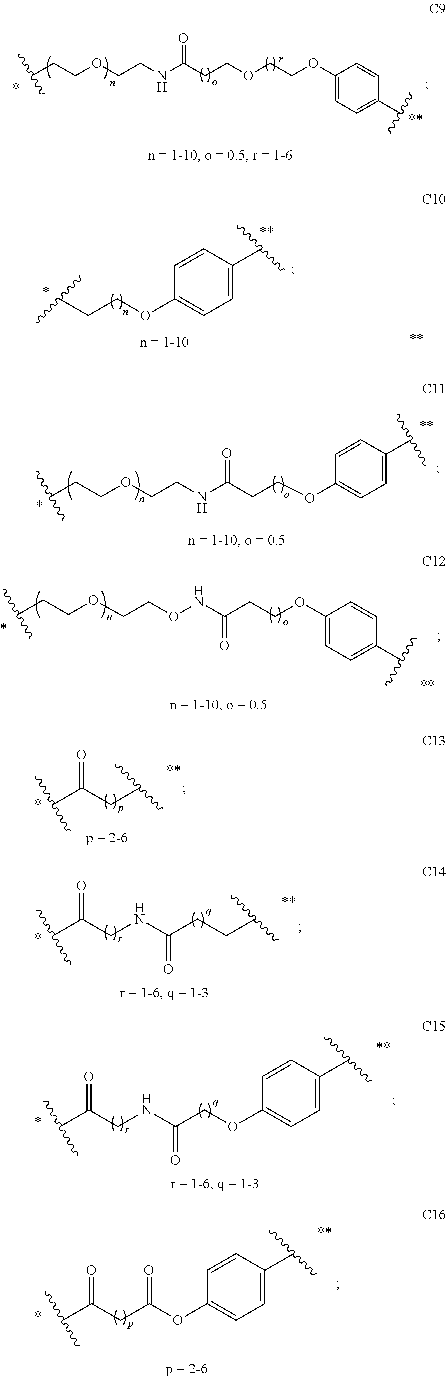

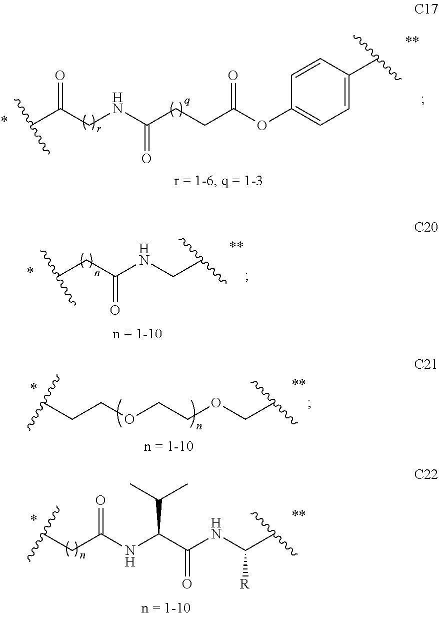

wherein R is methyl or

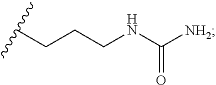 [SMILES: *CCCNC(N)=O]

wherein each oxygen is independently and optionally replaced by NH, NMe, NAc, S, or SO₂; * indicates the attachment point connecting to FG¹, ** indicates the attachment point connecting to the carbonyl or thiocarbonyl group, and the spacer without * or ** indicates each of the two attachment points can be connected to either FG¹ or the carbonyl or thiocarbonyl group;
each R¹ and R² is independently hydrogen or a C₁-C₆ alkyl;
or R¹ and R², together with the atom they are attached to, can form a 3-8 membered ring that can optionally contain one or more O, NMe, NAc, NSO₂Me, S, or SO₂;
a is an integer of 0 to 4;
each R^{e} is independently selected from nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, substituted or unsubstituted alkenyl, substituted or unsubstituted alkynyl, aryl or heteroaryl, and substituted aryl or heteroaryl;
Y¹ is O, NH or S;
Y² is O, NH or S;
FG¹ is a functional group capable of reacting through click chemistry; and
FG³ is a functional group selected from the group consisting of: —OH, —Cl, —Br, —I,

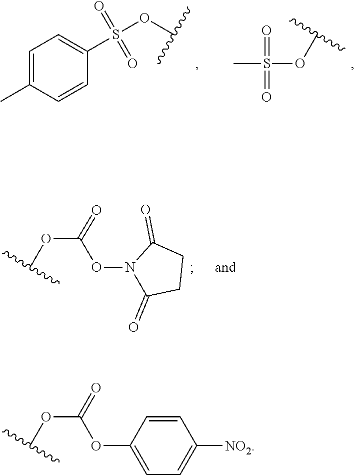

In certain embodiments, R¹ and R², together with the atom to which they are attached, can join together to form a 3-6 membered ring that can optionally contain one or more of O, NMe, NAc, NSO₂Me, S, or SO₂.

In certain embodiments of the releasable linker of formula (II), the releasable linker has a structure according to formula (II-B):

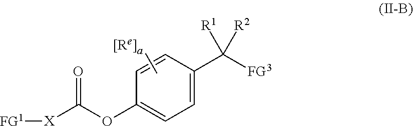

wherein:
X is selected from spacers C1 to C17 as described herein;
   wherein each oxygen is independently and optionally replaced by NH, NMe, NAc, S, or SO₂; * indicates the attachment point connecting to selected FG¹, ** indicates the attachment point connecting to the carbonyl group, and the spacer without * or ** indicates each of the two attachment points can be connected to either FG¹ or the carbonyl group;
R¹ is hydrogen, Me, or Et;
R² is hydrogen, Me, or Et;
a is an integer of 0 to 2;
each R^{e} is independently selected from the group consisting of nitro, cyano, halogen, —OMe, —NHMe, —NHAc, —NHSO₂Me, and —OCF₃;
FG¹ is a functional group capable of reacting through click chemistry; and
FG³ is a functional group selected from the group consisting of:
—OH, —Cl, —Br, —I,

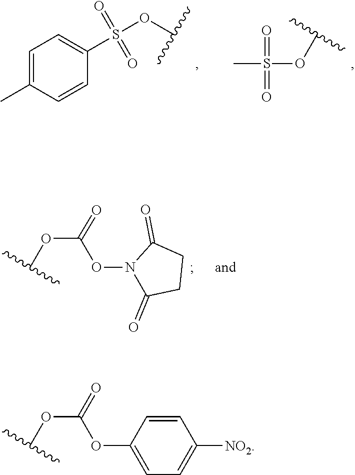

In certain embodiments, the releasable linker has a structure according to formula (II-C):

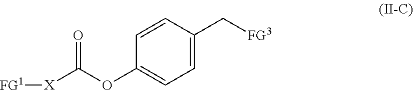

wherein:
X is selected from spacers C1, C3, C8, C10, or C13, as described herein, or C18 or C19:

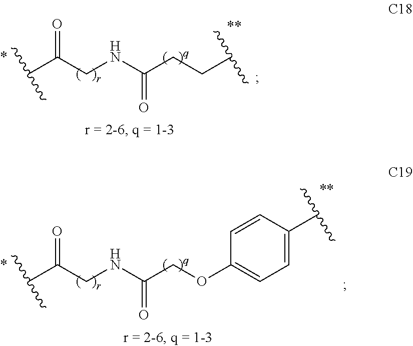

wherein * indicates the attachment point connecting to selected FG¹, ** indicates the attachment point connecting to the carbonyl group, and the spacer without * or ** indicates each of the two attachment points can be connected to either FG¹ or the carbonyl group;
FG¹ is an azide, dibenzocyclooctyne (DBCO), or alkynyl; and
FG³ is a functional group selected from the group consisting of:

### —OH, —Cl, —Br, —I,

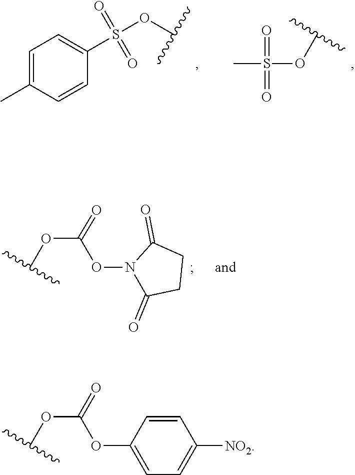

In certain embodiments of compound of formula (II-C), X is selected from spacers C3, C10, C13, C18 or C19 as described herein.

In certain embodiments, the releasable linker of formula (II) is:

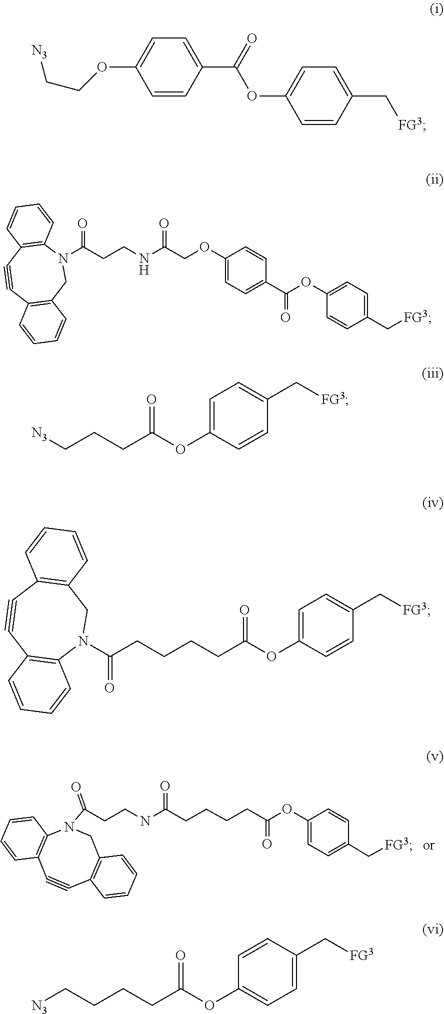

wherein each FG³ is independently —OH, —Cl, —I,

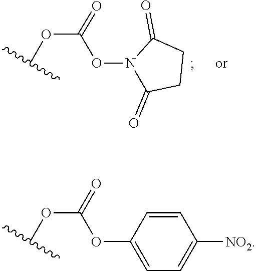 [SMILES: *OC(=O)ON1C(=O)CCC1=O ; *OC(=O)Oc1ccc([N+](=O)[O-])cc1]

In certain embodiments, the releasable linker of formula (II) has the following structures:

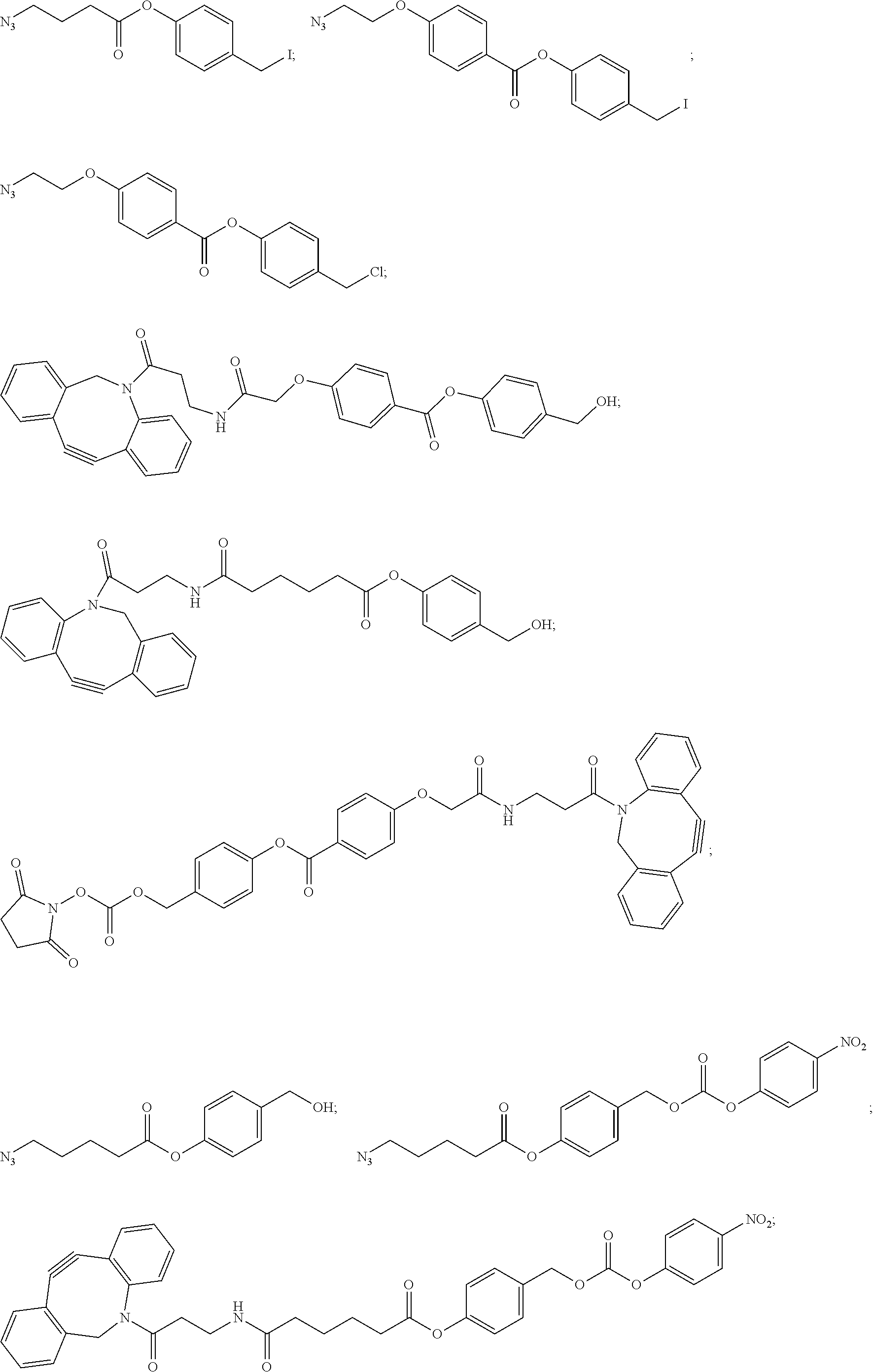

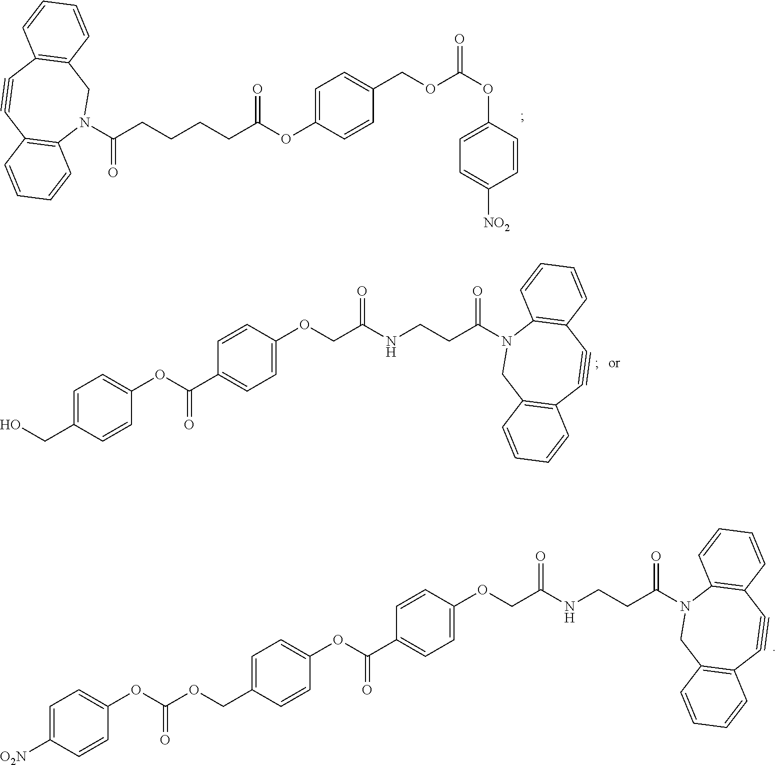 [SMILES: O=C(CCCCC(=O)N1Cc2ccccc2C#Cc2ccccc21)Oc1ccc(COC(=O)Oc2ccc([N+](=O)[O-])cc2)cc1 ; O=C(COc1ccc(C(=O)Oc2ccc(CO)cc2)cc1)NCCC(=O)N1Cc2ccccc2C#Cc2ccccc21 ; O=C(COc1ccc(C(=O)Oc2ccc(COC(=O)Oc3ccc([N+](=O)[O-])cc3)cc2)cc1)NCCC(=O)N1Cc2ccccc2C#Cc2ccccc21]

In certain embodiments, the present disclosure provides a linker with following structures:

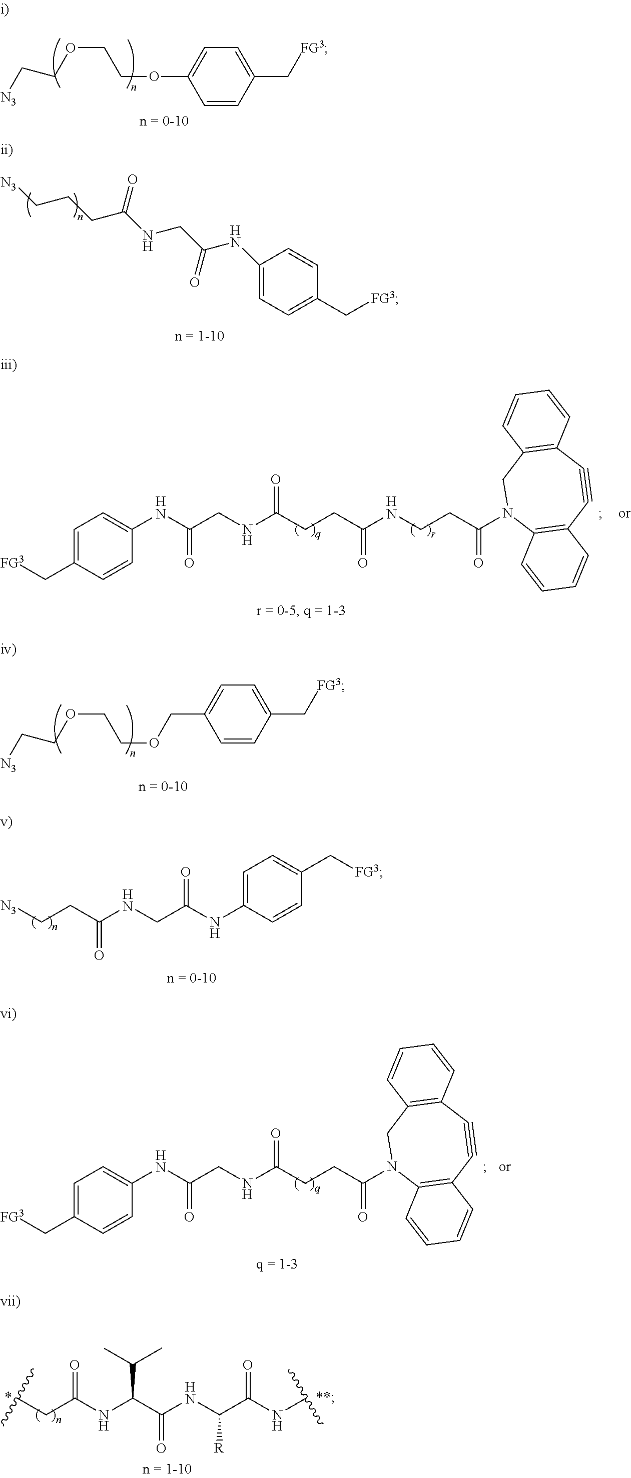

R is methyl or

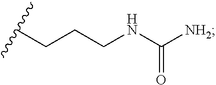 [SMILES: *CCCNC(N)=O]

wherein FG³ is independently —OH, —Cl, —I, or

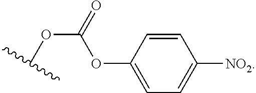 [SMILES: *OC(=O)Oc1ccc([N+](=O)[O-])cc1]

In certain embodiments, the linker has following structures:

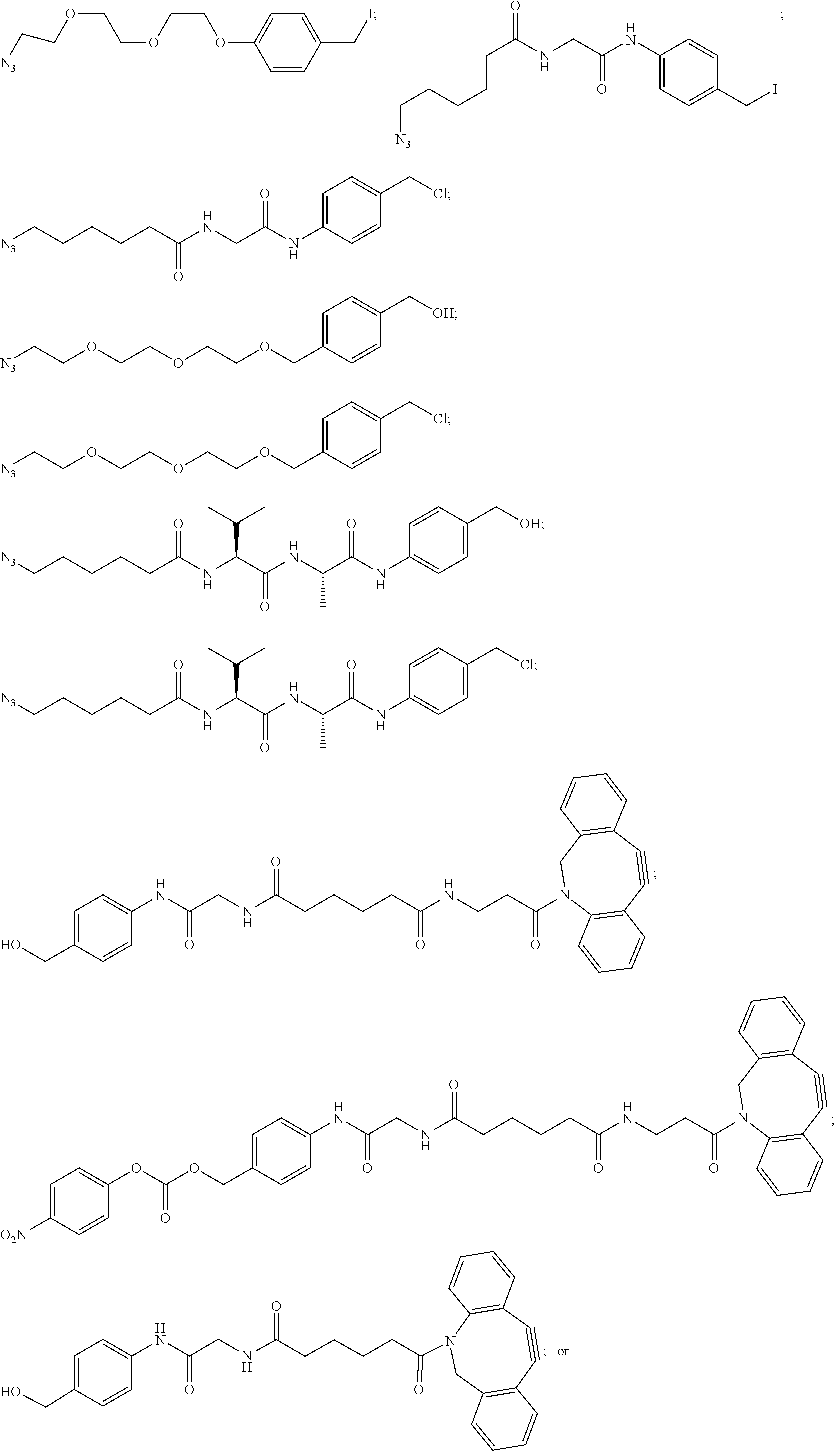

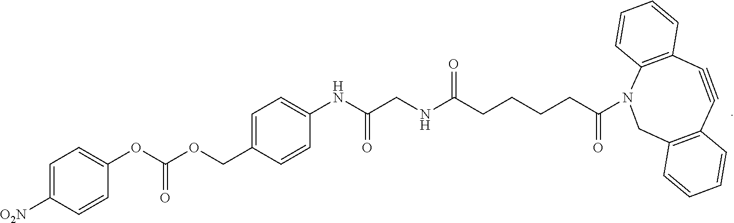 [SMILES: O=C(CCCCC(=O)N1Cc2ccccc2C#Cc2ccccc21)NCC(=O)Nc1ccc(COC(=O)Oc2ccc([N+](=O)[O-])cc2)cc1]

In certain embodiments, the present disclosure provides a compound comprising a releasable linker moiety covalently attached to a therapeutic agent, wherein the compound has a structure according to formula (III):

[Linker]_{b}-A; (III)

or an enantiomer, a mixture of enantiomers, a diastereomer, a mixture of two or more diastereomers, a tautomer, a mixture of two or more tautomers, or an isotopic variant thereof;
wherein
A is a therapeutic agent or an active moiety of a therapeutic agent; or a compound that decomposes to a therapeutic agent;
b is an integer of 1 or 2; wherein when b=2, both groups (inside the bracket, e.g., the Linker) are directly bound to A; and
each linker independently has a structure according to formula (III-L):

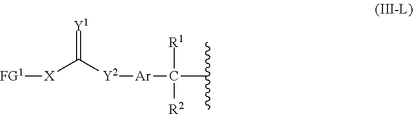

wherein:
Ar is substituted or unsubstituted aryl, or substituted or unsubstituted heteroaryl;
X is a spacer moiety;
each R¹ and R² is, independently, a hydrogen, substituted or unsubstituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
Y¹ is O or S;
Y² is O, NH or S;
FG¹ is a functional group capable of reacting through click chemistry; and
[symbol] represents a bond to A; wherein one or more atoms in each therapeutic agent or compound that decomposes to a therapeutic agent is independently replaced with a covalent bond to a linker, or a chemical group linking the therapeutic agent or compound to a linker.

In certain embodiments, in formula (III-L), R¹ and R², together with the atom to which they are attached, can join together to form a 3-8 membered ring that can optionally contain one or more O, NR^{XA} (wherein R^{XA} is alkyl, —C(O)-alkyl, or —S(O)₀₋₂-alkyl), or S(O)_{w} (wherein w is 0, 1, or 2). In certain embodiments, the R¹ and R², together with the atom to which they are attached, can join together to form a 3-8 membered ring that can optionally contain one or more of O, NMe, NAc, NSO₂Me, S, or SO₂.

An active moiety of a therapeutic agent refers to a moiety that is a specific part of a therapeutic agent that is responsible for characteristic activity of that agent. For example, and without being bound by this particular example, in one embodiment, CpG is the active moiety of the therapeutic agent TLR9 agonist of formula (I):

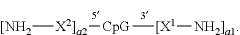

A compound that decomposes to a therapeutic agent refers to a chemical structure that can be readily converted into a therapeutic agent. For example, and without being bound by this particular example, in one embodiment, in formula (III), if A is

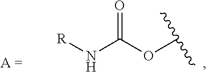

then RNH₂ is the therapeutic agent.

In certain embodiments, the compound of formula (III) is a compound of formula (III-A):

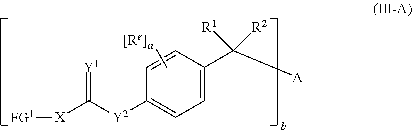

wherein:
each X is independently selected from spacers C1 to C17 or C20 to C22 as described herein:
wherein each oxygen is independently and optionally replaced by NH, NMe, NAc, S, or SO₂; * indicates the attachment point connecting to selected FG¹, ** indicates the attachment point connecting to the carbonyl or thiocarbonyl group, and the spacer without * or ** indicates each of the two attachment points can be connected to either FG¹ or the carbonyl or thiocarbonyl group;
each R¹ and R² is, independently, a hydrogen or a C₁-C₆ alkyl; or
R¹ and R² can join together, with the atom to which they are attached to form a 3-6 membered ring that can optionally contain one or more of O, NMe, NAc, NSO₂Me, S, or SO₂;
a is an integer of 0 to 4;
b is an integer of 1 or 2; wherein when b=2, both groups are directly bound to A;
each R^{e} is independently selected from nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, substituted or unsubstituted alkenyl, substituted or unsubstituted alkynyl, aryl or heteroaryl, and substituted aryl or heteroaryl;
Y¹ is O or S;
Y² is O, NH or S;
FG¹ is a functional group capable of reacting through click chemistry; and
A is a therapeutic agent or an active moiety of a therapeutic agent; or a compound that decomposes to a therapeutic agent.

In certain embodiments of the compound of formula (III), the compound is a compound of (III-B):

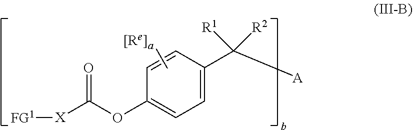

wherein:
each X is independently selected from spacers C1 to C17 as described herein;
   wherein each oxygen is independently and optionally replaced by NH, NMe, NAc, S, or SO₂; * indicates the attachment point connecting to selected FG¹, ** indicates the attachment point connecting to the carbonyl group, and the spacer without * or ** indicates each of the two attachment points can be connected to either FG¹ or the carbonyl group;
each R¹ and R² is independently hydrogen, Me, or Et;
a is an integer of 0 to 2;
b is an integer of 1 or 2; wherein when b=2, both groups are directly bound to A;
each R^{e} is independently selected from the group consisting of nitro, cyano, halogen, —OMe, —NHMe, —NHAc, —NHSO₂Me, and —OCF₃;
FG¹ is a functional group capable of reacting through click chemistry; and
A is a therapeutic agent or an active moiety of a therapeutic agent; or a compound that decomposes to a therapeutic agent.

In certain embodiments of the compound of formula (III), the compound is a compound of formula (III-C):

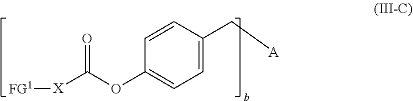

wherein:
each X is independently selected from spacers C1, C3, C8, C10, C13, C18 and C19 as described herein:
   wherein each oxygen is independently and optionally replaced by NH, NMe, NAc, S, or SO₂; * indicates the attachment point connecting to selected FG¹, ** indicates the attachment point connecting to the carbonyl group, and the spacer without * or ** indicates each of the two attachment points can be connected to either FG¹ or the carbonyl group;
b is an integer of 1 or 2; wherein when b=2, both groups are directly bound to A;
FG¹ is a functional group capable of reacting through click chemistry; and
A is a therapeutic agent or an active moiety of a therapeutic agent; or a compound that decomposes to a therapeutic agent.

In certain embodiments of compound of formula (III-C), each X is independently selected from spacers C3, C10, C13, C18 and C19 as described herein.

In certain embodiments, the present disclosure provides a compound comprising a linker moiety covalently attached to a therapeutic agent, wherein the compound has a structure according to formula (XXII):

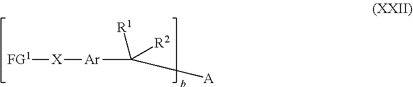

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X is a spacer moiety;
each R¹ and R² is, independently, a hydrogen, alkyl, substituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
FG¹ is a functional group capable of reacting through click chemistry;
b is an integer of 1 or 2; wherein when b=2, both groups are directly bound to A; and
A is a STING agonist; wherein one or more atoms in STING agonist is independently replaced with a covalent bond to a linker.

In certain embodiments of the compound of formula (III), the therapeutic agent is a TLR9 agonist.

In certain embodiments of the compound of formula (III), the therapeutic agent is a TLR7/8 agonist.

In certain embodiments of the compound of formula (III) or formula (XXII), the therapeutic agent is a STING agonist. Suitable STING agonists include those disclosed in WO 2019/043634 the contents of which are hereby incorporated by reference in its entirety. In certain embodiments, the STING agonist is an agonist disclosed in WO 2019/043634, ADU-S100, MK-1454, BMS-986301, GSK3745417, E7766, SB11285,

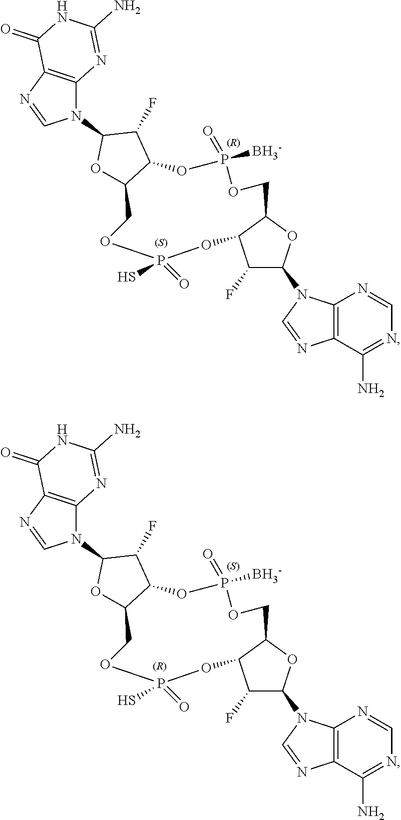

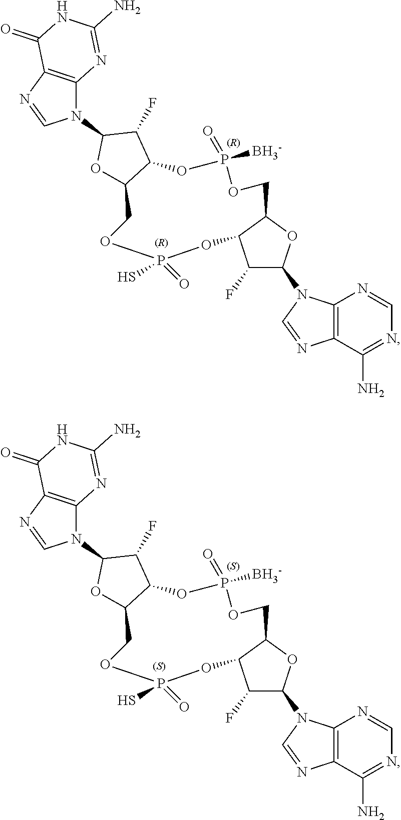

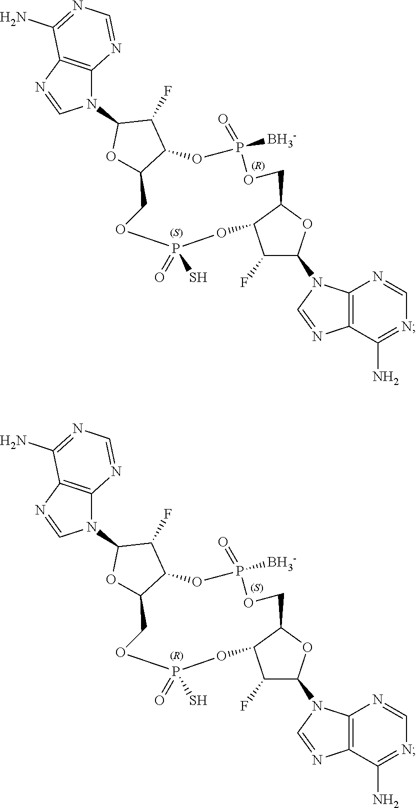

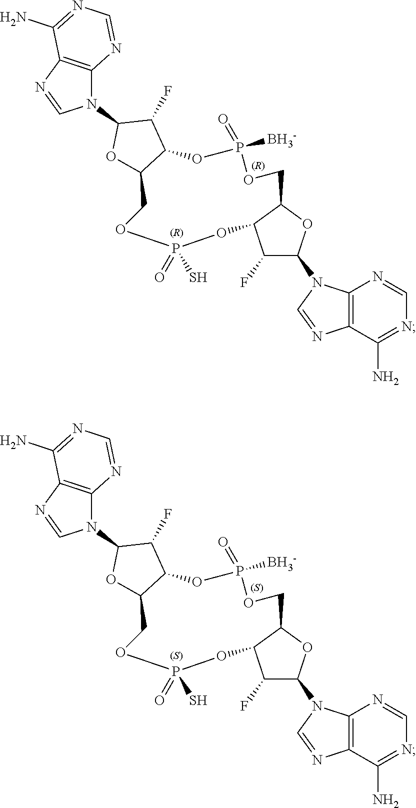

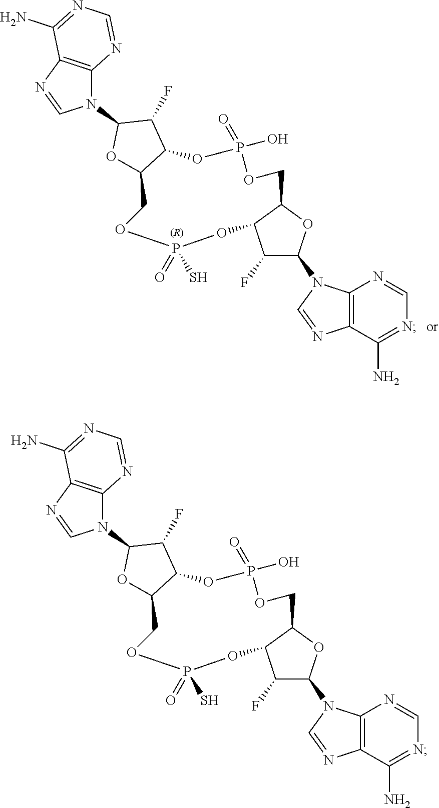

or a stereoisomer thereof.

In certain embodiments, the present disclosure provides a compound of formula (IV):

[Linker]_{b}-CDN; (IV)

or an enantiomer, a mixture of enantiomers, a diastereomer, a mixture of two or more diastereomers, a tautomer, a mixture of two or more tautomers, or an isotopic variant thereof;
wherein
CDN is a cyclic dinucleotide that is a STING agonist;
b is an integer of 1 or 2; wherein when b=2, both groups are directly bound to CDN; and
and each linker independently has a structure according to formula (IV-L):

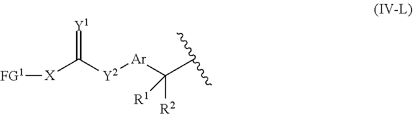

wherein:
X is a spacer moiety;
Ar is substituted or unsubstituted aryl, or substituted or unsubstituted heteroaryl;
each R¹ and R² is, independently, a hydrogen, alkyl, substituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
Y¹ is O or S;
Y² is O, NH or S;
FG¹ is a functional group capable of reacting through click chemistry; and
the linker is covalently bound to the cyclic dinucleotide STING agonist.

In certain embodiments of formula (IV), the linker is covalently bound to a thiol group (—SH) of the cyclic dinucleotide STING agonist (i.e., H of thiol is replaced with a covalent bond). In certain embodiments of formula (IV), the linker is covalently bound to a nitrogen of the cyclic dinucleotide STING agonist (by replacing an amino H with a covalent bond).

In certain embodiments of the linker of formula (IV-L), R¹ and R², together with the atom to which they are attached, can join together to form a 3-8 membered ring that can optionally contain one or more O, NR^{XA} (wherein R^{XA} is alkyl, —C(O)-alkyl, or —S(O)₀₋₂-alkyl), or S(O)_{w} (wherein w is 0, 1, or 2). In certain embodiments, of the linker of formula (IV-L), R¹ and R², together with the atom to which they are attached, can join together to form a 3-8 membered ring that can optionally contain one or more O, NMe, NAc, NSO₂Me, S, or SO₂.

In certain embodiments of the compound of formula (IV), the compound is a compound of formula (IV-A):

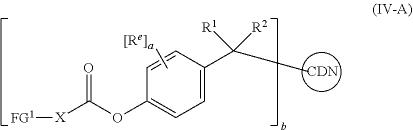

wherein:
each X is independently selected from spacers C1 to C17 as described herein:
   wherein each oxygen is independently and optionally replaced by NH, NMe, NAc, S, or SO₂; * indicates the attachment point connecting to selected FG¹, ** indicates the attachment point connecting to the carbonyl group, and the spacer without * or ** indicates each of the two attachment points can be connected to either FG¹ or the carbonyl group;
   each R¹ and R² is independently, hydrogen, Me, or Et;
   a is an integer of 0 to 2;
   b is an integer of 1 or 2; wherein when b=2, both groups are directly bound to CDN;
   each R^{e} is independently selected from the group consisting of nitro, cyano, halogen, —OMe, —NHMe, —NHAc, —NHSO₂Me, and —OCF₃;
   FG¹ is a functional group capable of reacting through click chemistry;
   CDN is a cyclic dinucleotide that is a STING agonist; and
   the linker is covalently bound to the cyclic dinucleotide STING agonist.

In certain embodiments of formula (IV-A), the linker is covalently bound to a thiol group of the cyclic dinucleotide STING agonist. In certain embodiments of formula (IV-A), the linker is covalently bound to a nitrogen of the cyclic dinucleotide STING agonist.

In certain embodiments of the compound of formula (IV), the compound is a compound of formula (IV-B):

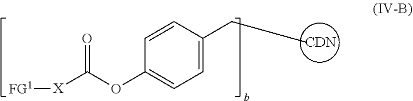

wherein:
X is selected from spacers C1, C3, C8, C10, C13, C18 and C19 as described herein:
   wherein * indicates the attachment point connecting to selected FG¹, ** indicates the attachment point connecting to the carbonyl group, and the spacer without * or ** indicates each of the two attachment points can be connected to either FG¹ or the carbonyl group;
b is an integer of 1 or 2; wherein when b=2, both groups are directly bound to CDN;
FG¹ is an azide, dibenzocyclooctyne (DBCO), or alkynyl;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments of the compound of formula (IV-A) or (IV-B), wherein X is selected from spacers C1, C3, C8, and C10 as described herein; wherein * indicates the attachment point connecting to FG¹, ** indicates the attachment point connecting to the carbonyl group, and the spacer without * or ** indicates the attachment points can be connected to either FG¹ or the carbonyl group; b is an integer of 1 or 2; wherein when b=2, both groups are directly bound to CDN; FG¹ is an azide; CDN is a cyclic dinucleotide that is a STING agonist; and the linker is covalently bound to a thiol group of the cyclic dinucleotide STING agonist. In certain embodiments, X is C3 or C10. In certain embodiments, n is 1 or 2.

In certain embodiments, the present disclosure provides a compound of formula (XXIII):

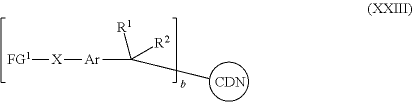

or an enantiomer, a mixture of enantiomers, a diastereomer, a mixture of two or more diastereomers, a tautomer, a mixture of two or more tautomers, or an isotopic variant thereof;
wherein:
X is a spacer moiety;
Ar is substituted or unsubstituted aryl, or substituted or unsubstituted heteroaryl;
each R¹ and R² is, independently, a hydrogen, alkyl, substituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
FG¹ is a functional group capable of reacting through click chemistry;
CDN is a cyclic dinucleotide that is a STING agonist; and
the linker is covalently bound to an atom or a group of the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments of the compound of formula (XXIII), the compound is a compound of formula (XXIII-A):

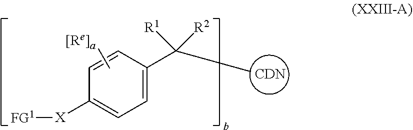

wherein:
X is:

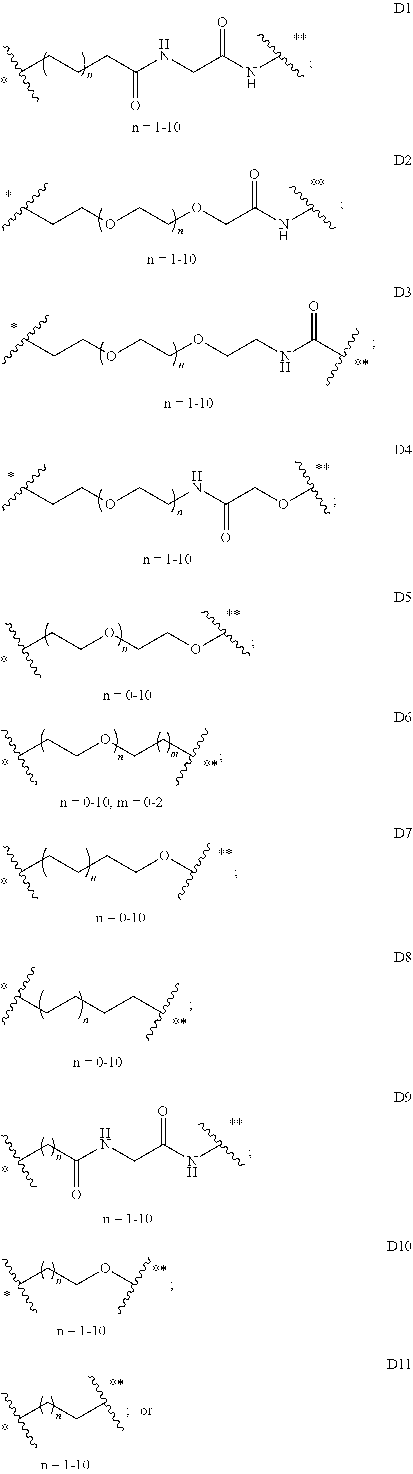

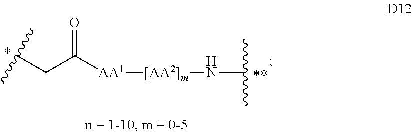

wherein AA¹-[AA²]ₘ comprises the groups selected from: Gly, Lys, Val-Ala, Val-Lys, Val-Cit, Ala-Lys, Phe-Lys, Phe-Cit, Leu-Cit, Ile-Cit, Trp-Cit, Phe-Arg, Phe-Leu, Ala-Phe, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Pro-(D)Lys, Met-Cit-Val, Gly-Cit-Val, Phe-Phe-Lys, (D)Phe-Phe-Lys, (D)Ala-Phe-Lys, Gly-Phe-Lys, Glu-Val-Cit, Ser-Val-Cit, Gly-Phe-Leu-Gly, and Ala-Leu-Ala-Leu;
   * indicates the attachment point connecting to FG¹, ** indicates the attachment point connecting to the phenyl group, and the spacer without * or ** indicates the attachment points can be connected to either FG¹ or the phenyl group;
each R¹ and R² is independently, hydrogen, Me, or Et;
a is an integer of 0 to 2;
b is an integer of 1 or 2; wherein when b=2, both groups are directly bound to CDN;
each R^{e} is independently selected from the group consisting of nitro, cyano, halogen, —OMe, —NHMe, —NHAc, —NHSO₂Me, and —OCF₃;
FG¹ is a functional group capable of reacting through click chemistry;
CDN is a cyclic dinucleotide that is a STING agonist; and
the linker is covalently bound to a thiol group of the cyclic dinucleotide STING agonist.

As used herein, amino acid “Cit” refers to Citrulline and amino acid “NorVal” refers to Norvaline.

In certain embodiments of the compound of formula (XXIII), the compound is a compound of formula (XXIII-B):

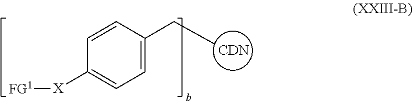

wherein:
X is selected from spacer D1, D5, D6, or D9 as described herein, or D13:

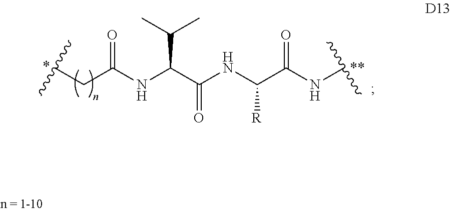

wherein R is methyl or

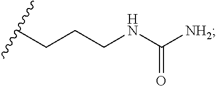 [SMILES: *CCCNC(N)=O]

wherein * indicates the attachment point connecting to FG¹, ** indicates the attachment point connecting to the phenyl group;
b is an integer of 1 or 2; wherein when b=2, both groups are directly bound to CDN;
FG¹ is an azide;
CDN is a cyclic dinucleotide that is a STING agonist; and
the linker is covalently bound to a thiol group of the cyclic dinucleotide STING agonist.

In certain embodiments of compound of formula (XXIII-B), R is methyl for spacer D13 as described herein.

In certain embodiments of the compound of formula (III), (XXII), (IV) and (XXIII), the cyclic dinucleotide STING agonist is:

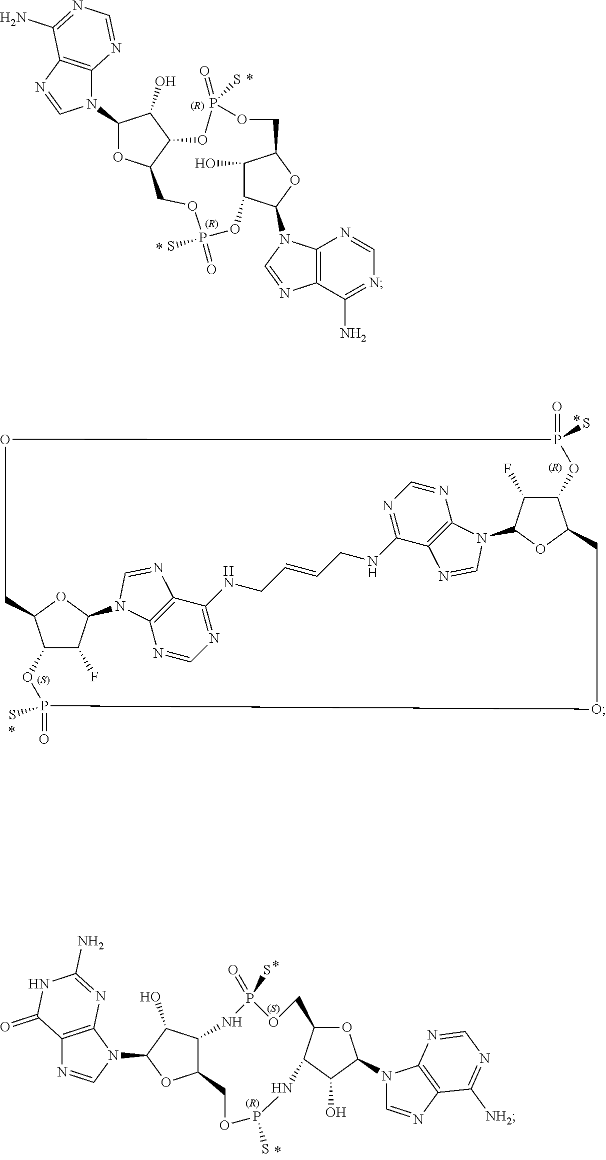

or a stereoisomer thereof;
wherein the * indicates the thiol group of the cyclic dinucleotide that is connecting with the linker. Where there is only one linker connected to the STING agonist shown above, the remaining S* is SH.

In some embodiments of the compound of formula (III), (IV), (XXII), and (XXIII), the cyclic dinucleotide STING agonist is:

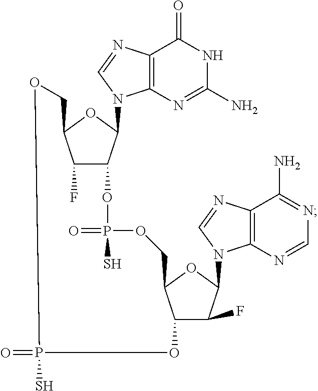 [SMILES: Nc1nc2c(ncn2[C@@H]2O[C@@H]3CO[P@@](=O)(S)O[C@H]4[C@H](F)[C@H](n5cnc6c(N)ncnc65)O[C@@H]4CO[P@@](=O)(S)O[C@@H]2[C@@H]3F)c(=O)[nH]1]

or a stereoisomer thereof, wherein any atom or group of the STING agonist is replaced to form a covalent bond with the rest of the compound, including H of —SH and —NH₂.

In certain embodiments of the compound of formula (III), (XXII), (IV) and (XXIII), b is one. In certain embodiments of the compound of formula (III), (XXII), (IV) and (XXIII), b is two.

In certain embodiments of the compound of formula (III), (XXII), (IV) and (XXIII), when b is one, the cyclic dinucleotide STING agonist is:

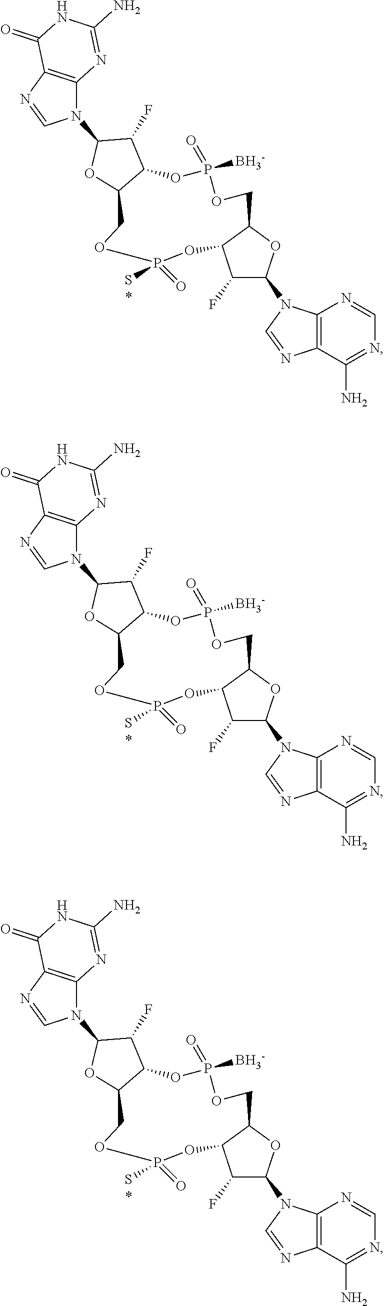

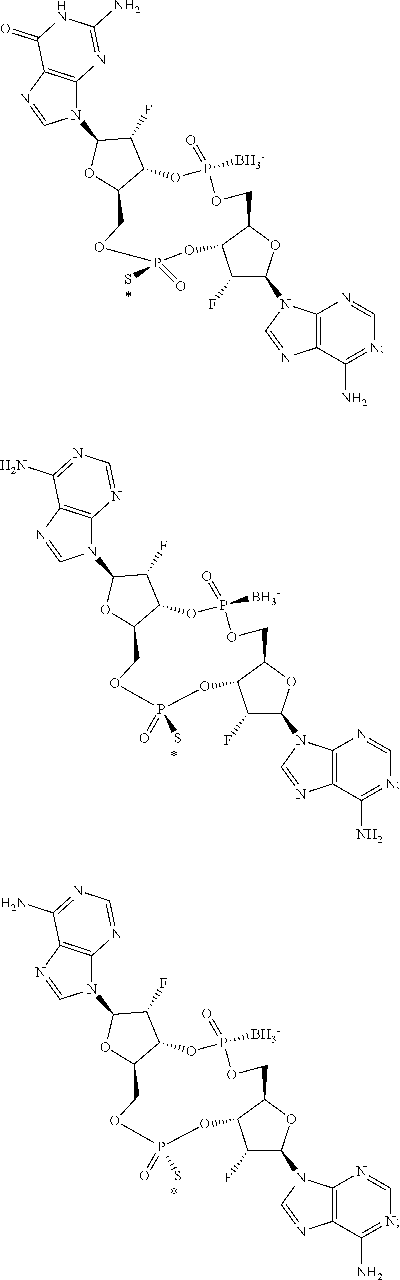

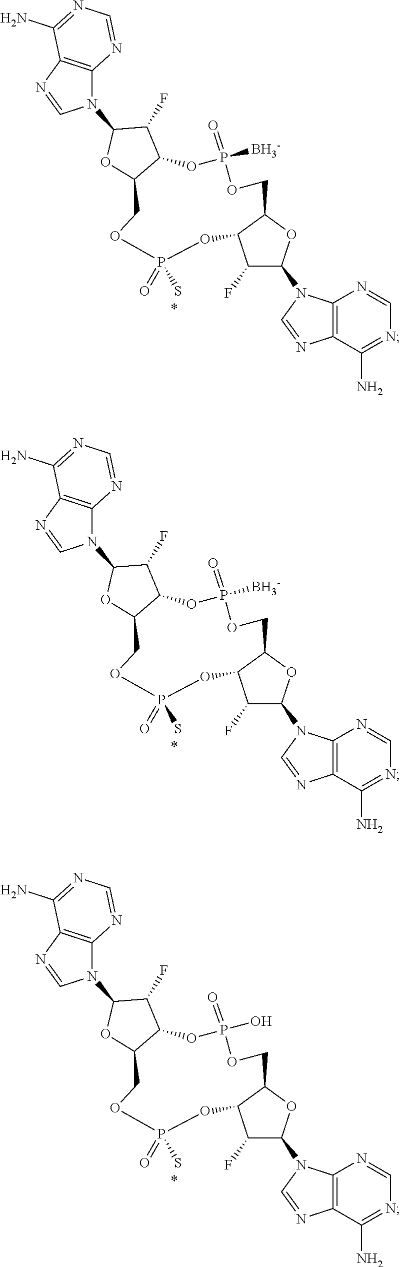

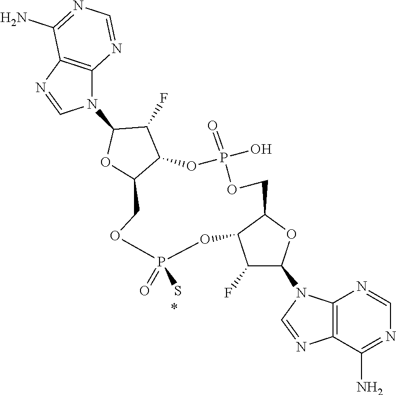 [SMILES: Nc1ncnc2c1ncn2[C@@H]1O[C@@H]2CO[P@](=O)(S)O[C@H]3[C@@H](F)[C@H](n4cnc5c(N)ncnc54)O[C@@H]3COP(=O)(O)O[C@H]2[C@H]1F]

or a stereoisomer thereof;
wherein the * indicates the thiol group of the cyclic dinucleotide that is connecting with the linker.

In certain embodiments of the compound of formula (III), (IV), (XXII) and (XXIII), FG¹ is an azide.

In certain embodiments of the compound of formula (IV) and (XXIII), n is 2.

In certain embodiments of the compounds of formula (IV), (IV-A), (IV-B), (XXIII), (XXIII-A), and (XXIII-B), the compound is selected from:

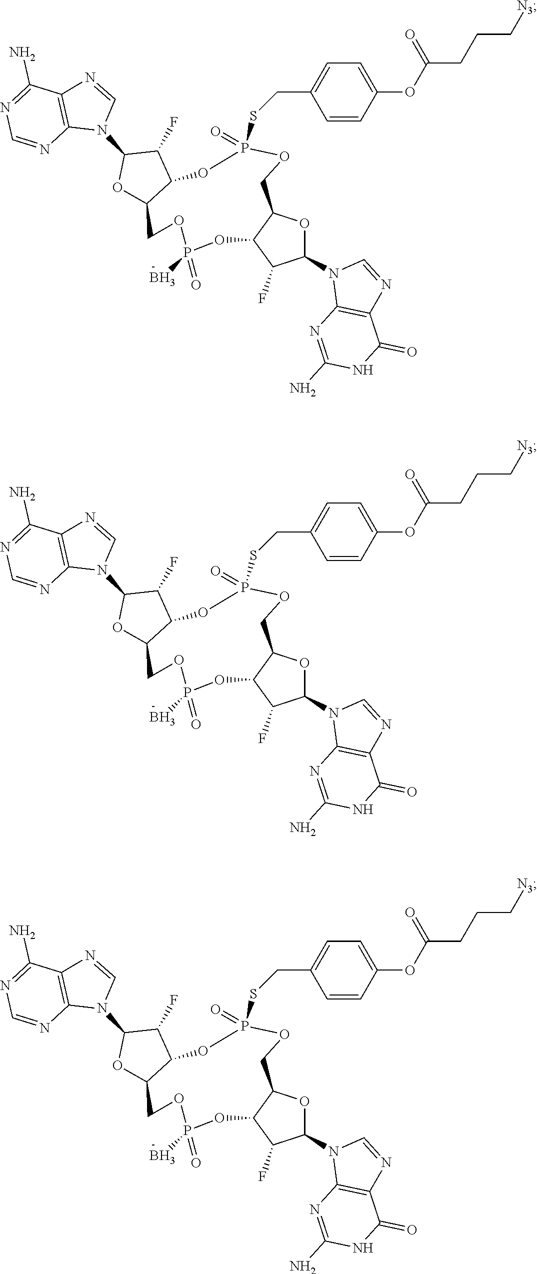

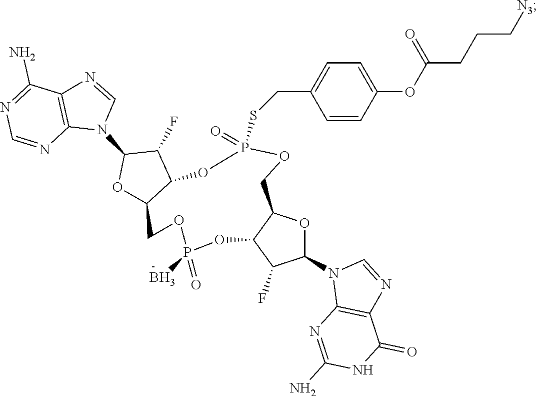

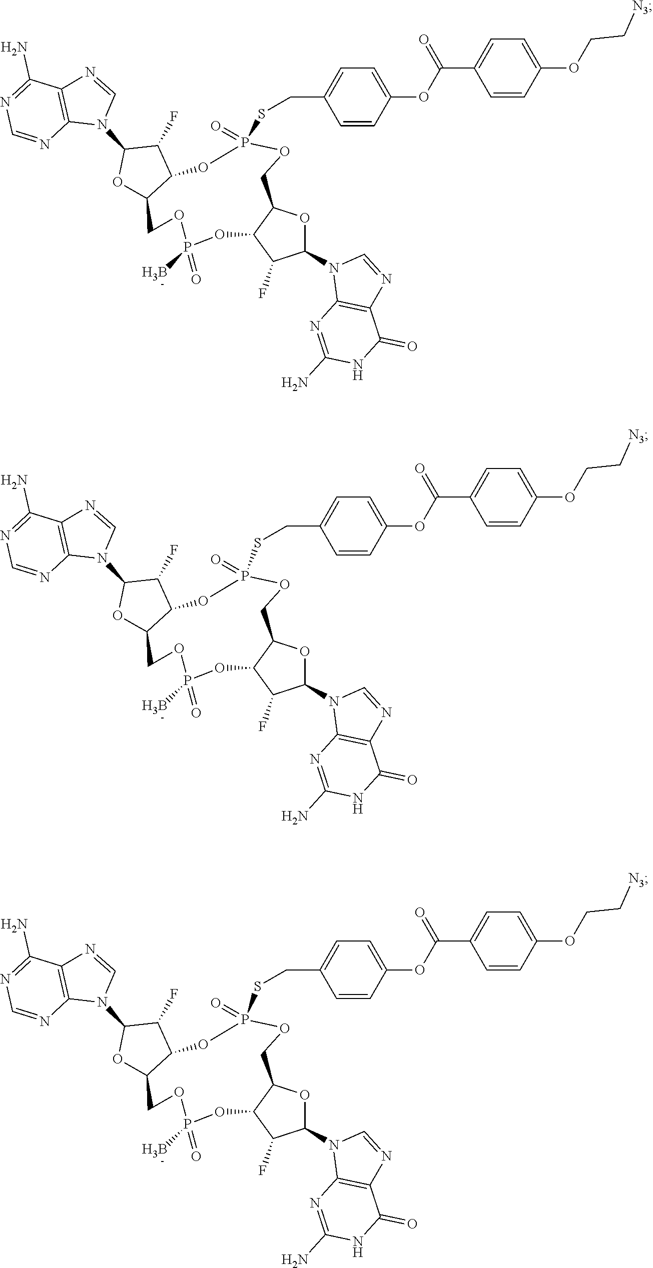

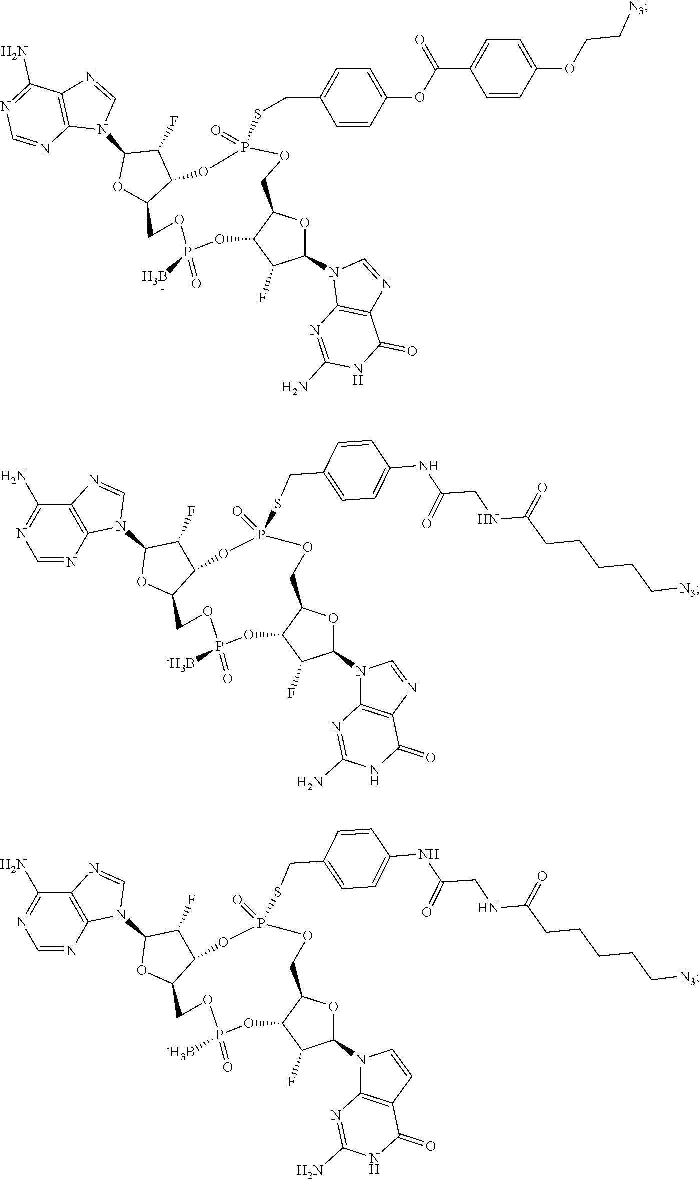

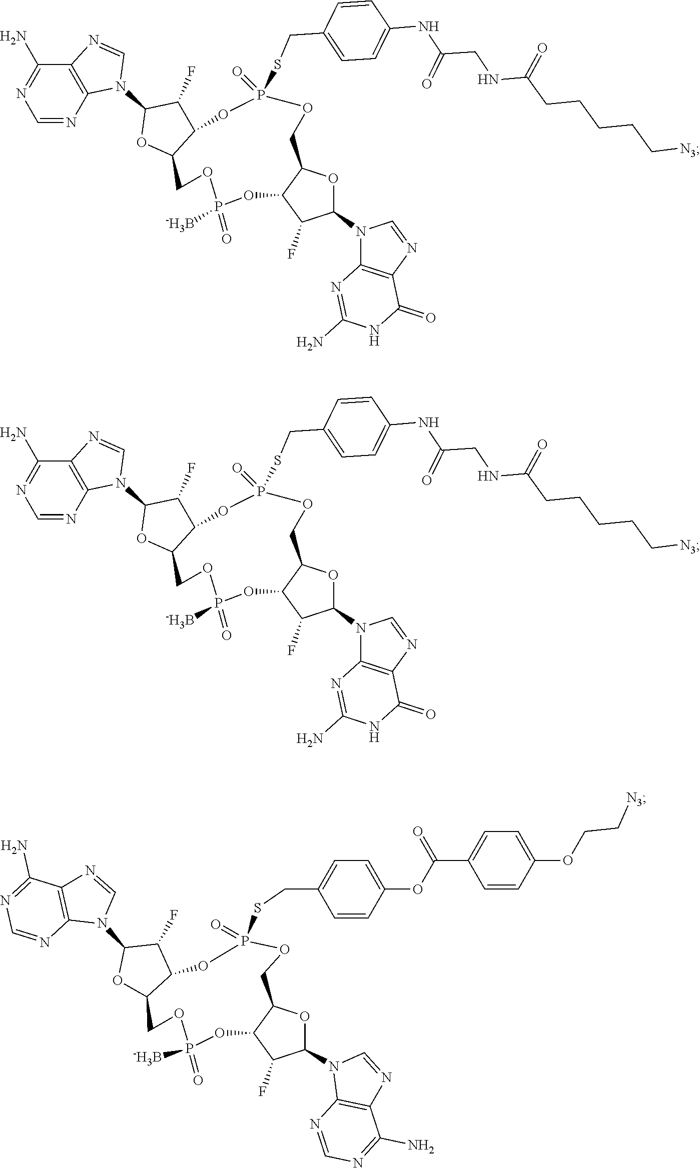

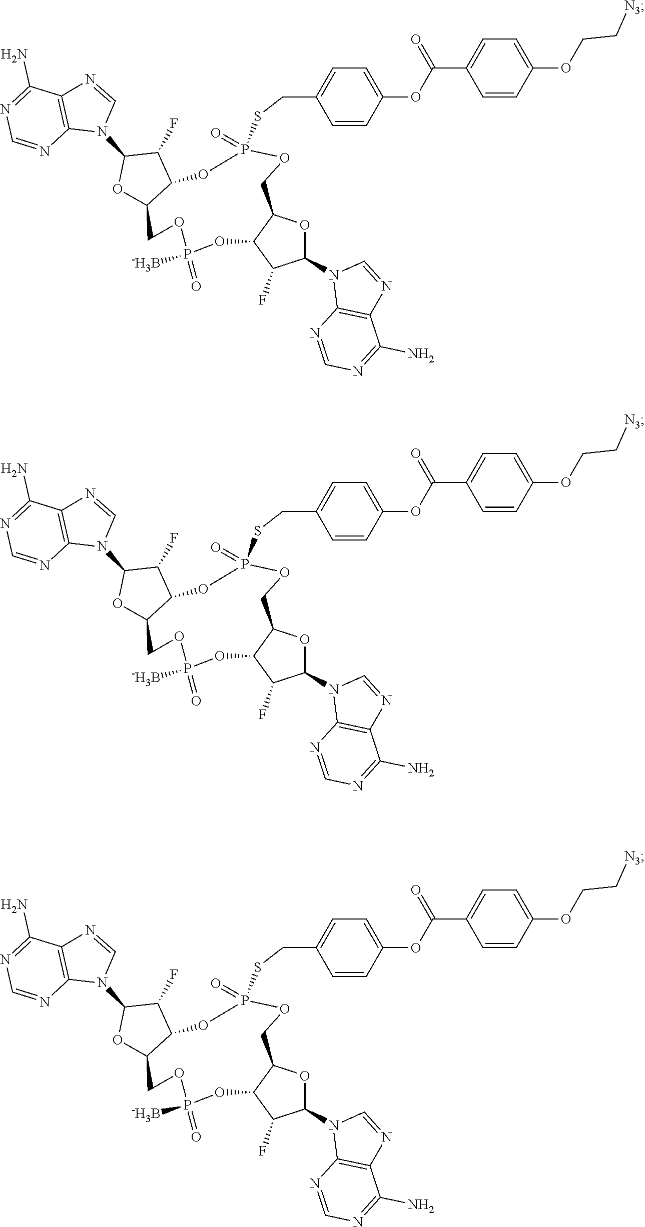

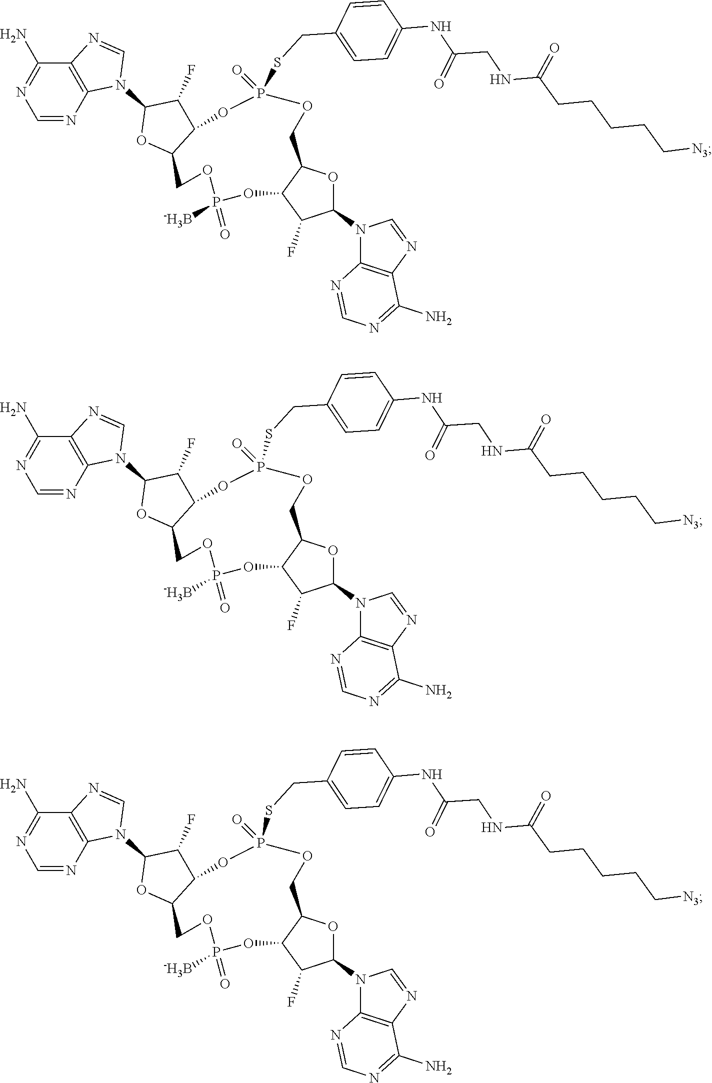

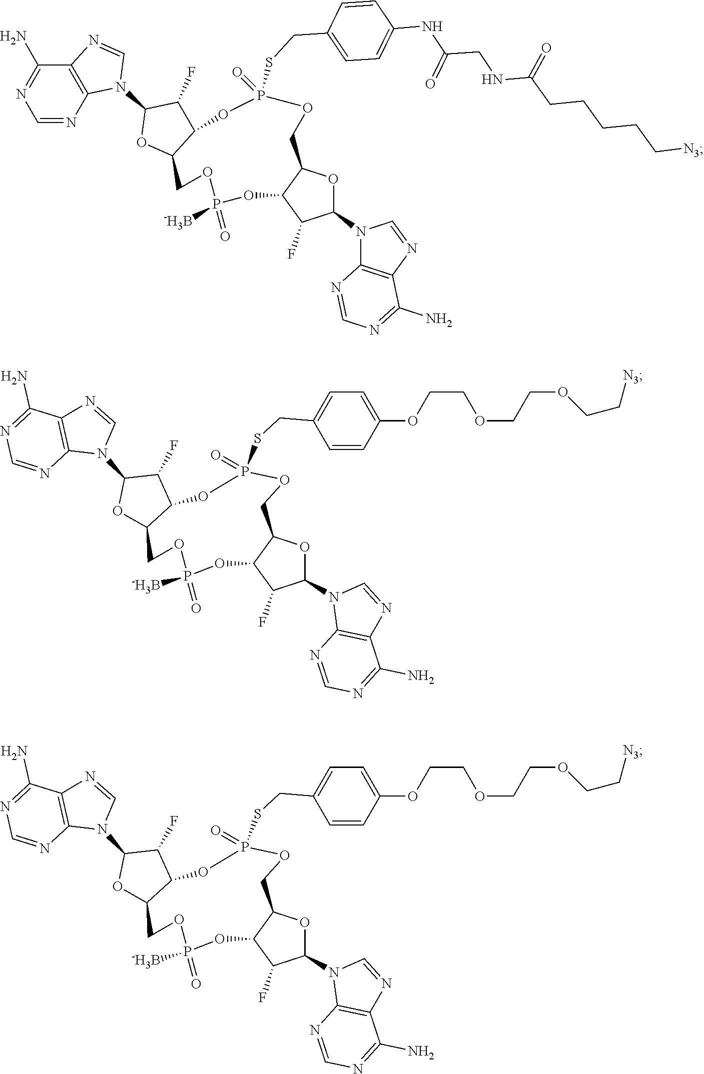

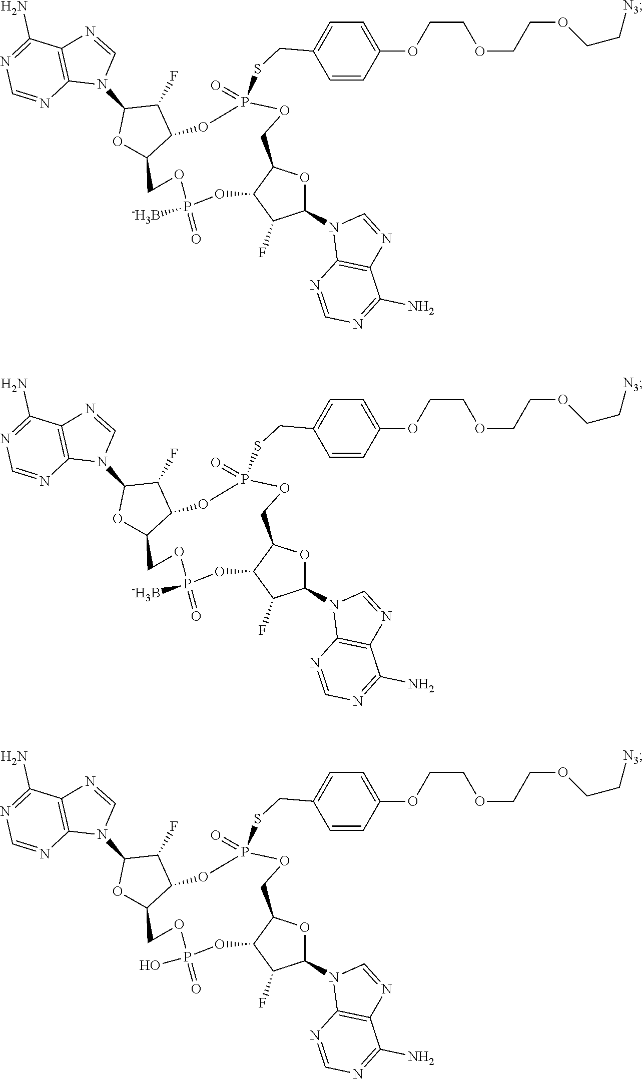

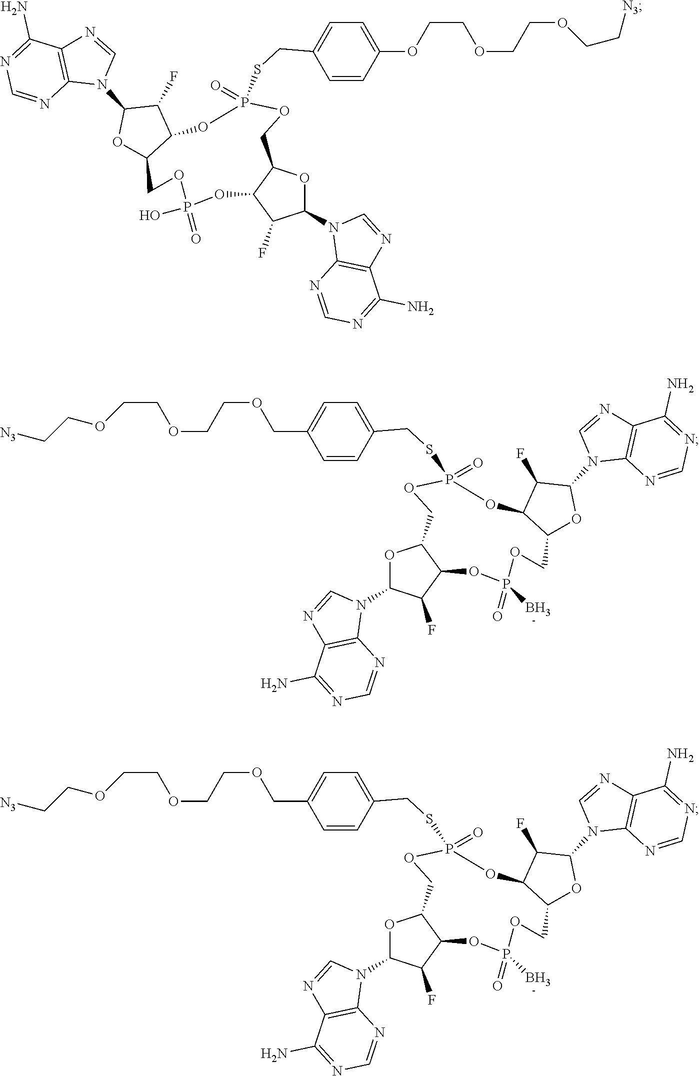

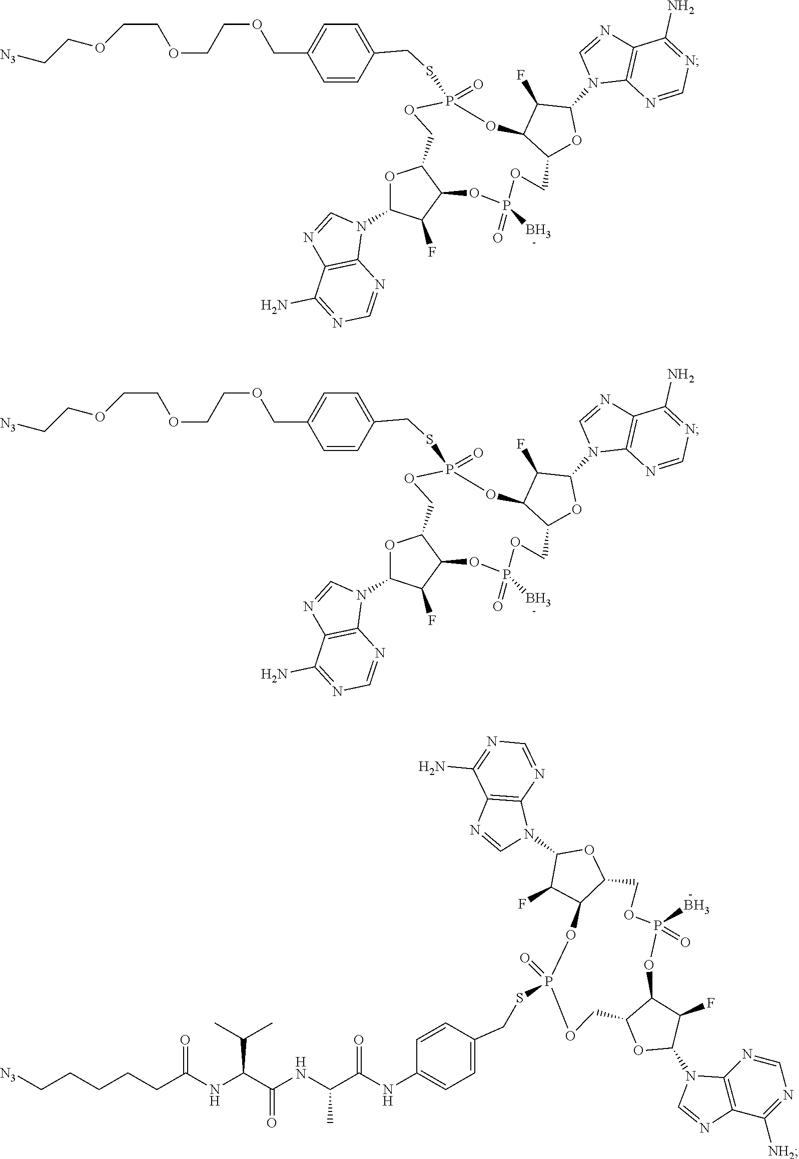

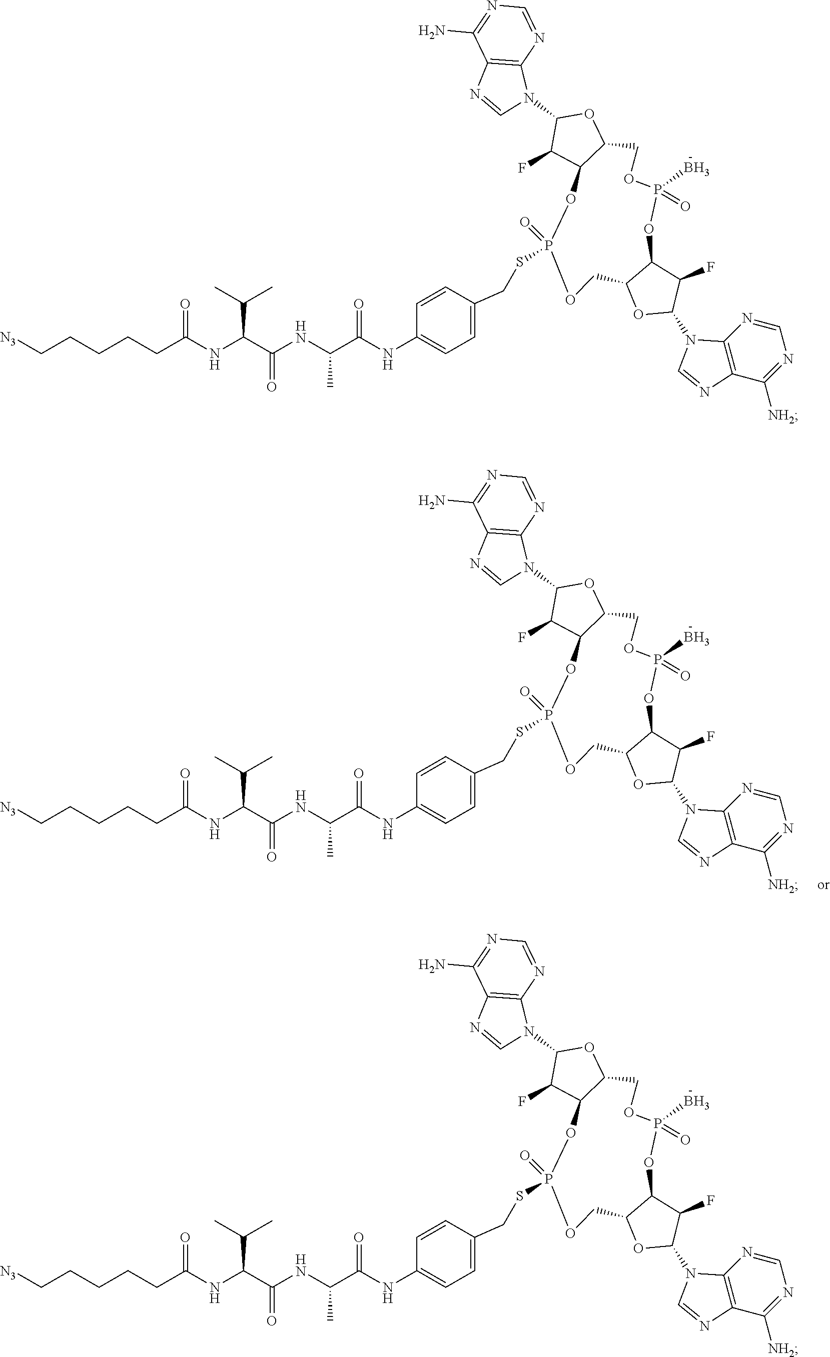

or a stereoisomer thereof.

In certain embodiments, the present disclosure provides a compound of formula (V):

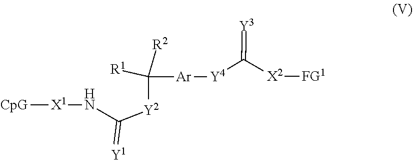

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X¹ and X² are each independently a spacer moiety;
each R¹ and R² is independently, a hydrogen, substituted or unsubstituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
Y¹ is O, NH or S;
Y² is O, NH or S;
Y³ is O, NH or S;
Y⁴ is O, NH or S;
FG¹ is a functional group capable of reacting through click chemistry; and
CpG is a TLR9 agonist oligodeoxynucleotide wherein the CpG is covalently bound to X¹ through 3′-O or 5′-O of a terminal nucleotide;

In certain embodiments of the compound of formula (V), R¹ and R², together with the atom to which they are attached, can join together to form a 3-8 membered ring that can optionally contain one or more O, NR^{XA} (wherein R^{XA} is alkyl, —C(O)-alkyl, or —S(O)₀₋₂-alkyl), or S(O)_{w} (wherein w is 0, 1, or 2). In certain embodiments of the compound of formula (V), R¹ and R², together with the atom to which they are attached, can join together to form a 3-8 membered ring that can optionally contain one or more O, NMe, NAc, NSO₂Me, S, or SO₂.

In certain embodiments of the compound of formula (V), the compound is a compound of formula (V-A):

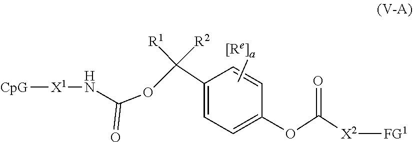

wherein:
X¹ is **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; ** indicates the attachment point connecting to the amino group;
X² is selected from spacers C1 to C17 as described herein:
   wherein each oxygen is independently and optionally replaced by NH, NMe, NAc, S, or SO₂; * indicates the attachment point connecting to selected FG¹, ** indicates the attachment point connecting to the carbonyl group, and the spacer without * or ** indicates each of the two attachment points can be connected to either FG¹ or the carbonyl group
   each R¹ and R² is independently hydrogen, Me, or Et;
   a is an integer of 0 to 2;
   each R^{e} is independently selected from the group consisting of nitro, cyano, halogen, —OMe, —NHMe, —NHAc, —NHSO₂Me, and —OCF₃;
   FG¹ is a functional group capable of reacting through click chemistry; and
   CpG is a TLR9 agonist oligodeoxynucleotide wherein the CpG is covalently bound to X¹ through a 3′-O or 5′-O of a terminal nucleotide.

In certain embodiments of the compound of formula (V), the compound is a compound of formula (V-B):

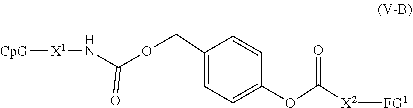

wherein:
X¹ is selected from **—C₃-C₁₂ alkylene-L¹-*; or spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; ** indicates the attachment point connecting to the amino group;
X² is selected from spacers C1, C3, C8, C10, C13, C18 and C19 as described herein:
   wherein * indicates the attachment point connecting to selected FG¹, ** indicates the attachment point connecting to the carbonyl group, and the spacer without * or ** indicates each of the two attachment points can be connected to either FG¹ or the carbonyl group;
FG¹ is an azide, dibenzocyclooctyne (DBCO), or alkynyl; and
CpG is a TLR9 agonist oligodeoxynucleotide wherein the CpG is covalently bound to X¹ through a 3′-O or 5′-O of a terminal nucleotide.

In certain embodiments of the compound of formula (V-B), wherein X¹ is spacer B3 as described herein: wherein L¹ is independently —OP(O)(OH)—, or —OP(S)(OH)—; * indicates the attachment point connecting to a 3′-O of a terminal nucleotide of the CpG; and ** indicates the attachment point connecting to the amino group; X² is spacer C19 as described herein: wherein * indicates the attachment point connecting to FG¹, and ** indicates the attachment point connecting to the carbonyl group; and FG¹ is dibenzocyclooctyne (DBCO). In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101); wherein the CpG is covalently bound to X¹ through a 3′-O of a terminal nucleotide of the CpG.

In certain embodiments of the compound of formula (V), (V-A), or (V-B), X¹ is B3 and o is 1. In certain embodiments of the compound of formula (V), (V-A), or (V-B), X² is C19; q is 1; and r is 2.

In certain embodiments of the compound of formula (V), (V-A), or (V-B), the compound is

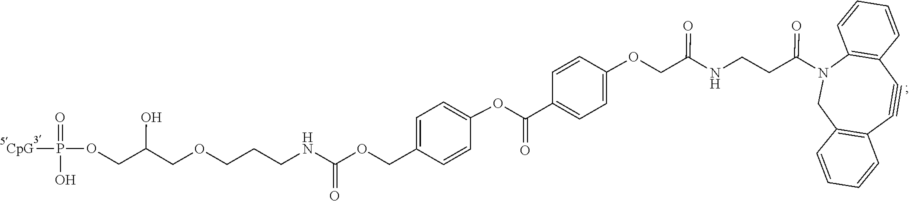

wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide.

In certain embodiments, the present disclosure provides a compound of formula (VI):

CpG-X-FG¹ (VI)

or an enantiomer, a mixture of enantiomers, a diastereomer, a mixture of two or more diastereomers, a tautomer, a mixture of two or more tautomers, or an isotopic variant thereof;
wherein:
X is a spacer moiety;
FG¹ is a functional group capable of reacting through click chemistry; and
CpG is a TLR9 agonist oligodeoxynucleotide wherein the CpG is covalently bound to X¹ through 3′-O or 5′-O of a terminal nucleotide of the CpG.

In certain embodiments of the compound of formula (VI), X is —X¹—NH—CO—X²—, wherein: X¹ is **—C₃-C₁₂ alkylene-L¹-*; or spacers B1 to B5 or B6 or B7 as described herein:

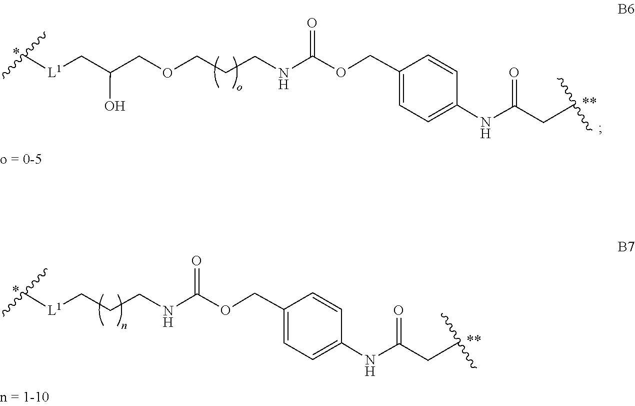

wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
X² is selected from spacers C1, C3, C8, C10, C13, C18 and C19 as described herein: wherein * indicates the attachment point connecting to selected FG¹, ** indicates the attachment point connecting to the carbonyl group, and the spacer without * or ** indicates each of the two attachment points can be connected to either FG¹ or the carbonyl group;
FG¹ is an azide, dibenzocyclooctyne (DBCO), or alkynyl.

In certain embodiments of the compound of formula (VI), X is —X¹—NH—CO—X²—, wherein: X¹ is selected from spacers B3 and B6 as described herein: wherein L¹ is independently —OP(O)(OH)—, or —OP(S)(OH)—; * indicates the attachment point connecting to a 3′-O of a terminal nucleotide of the CpG; and ** indicates the attachment point connecting to the amino group; or X¹ is: **—C₃-C₁₂ alkylene-L¹-*, or B7 as described herein, wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; X² is selected from spacers C1, C3, C13 and C18 as described herein: wherein * indicates the attachment point connecting to selected FG¹, ** indicates the attachment point connecting to the carbonyl group, and the spacer without * or ** indicates each of the two attachment points can be connected to either FG¹ or the carbonyl group; FG¹ is an azide, or dibenzocyclooctyne (DBCO). In certain embodiments, CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ (SD-101), connecting at 3′-O or 5′-O of the terminal nucleotide.

In certain embodiments of the compound of formula (VI), X¹ is B3 or B6. In embodiments, X¹ is B3 or B6, and o is 1. In embodiments, X² is C3 or C13. In embodiments, X² is C3 or C13; n is 4; and p is 4. In embodiments, X² is C18. In embodiments, X² is C18; q is 3; and r is 2.

In some embodiments of the compound of formula (VI), X¹ is B7 wherein * indicates the attachment point connecting to a 5′-O of a terminal nucleotide of the CpG; and ** indicates the attachment point connecting to the amino group.

In certain embodiments of the compound of formula (VI), the compound is represented by:

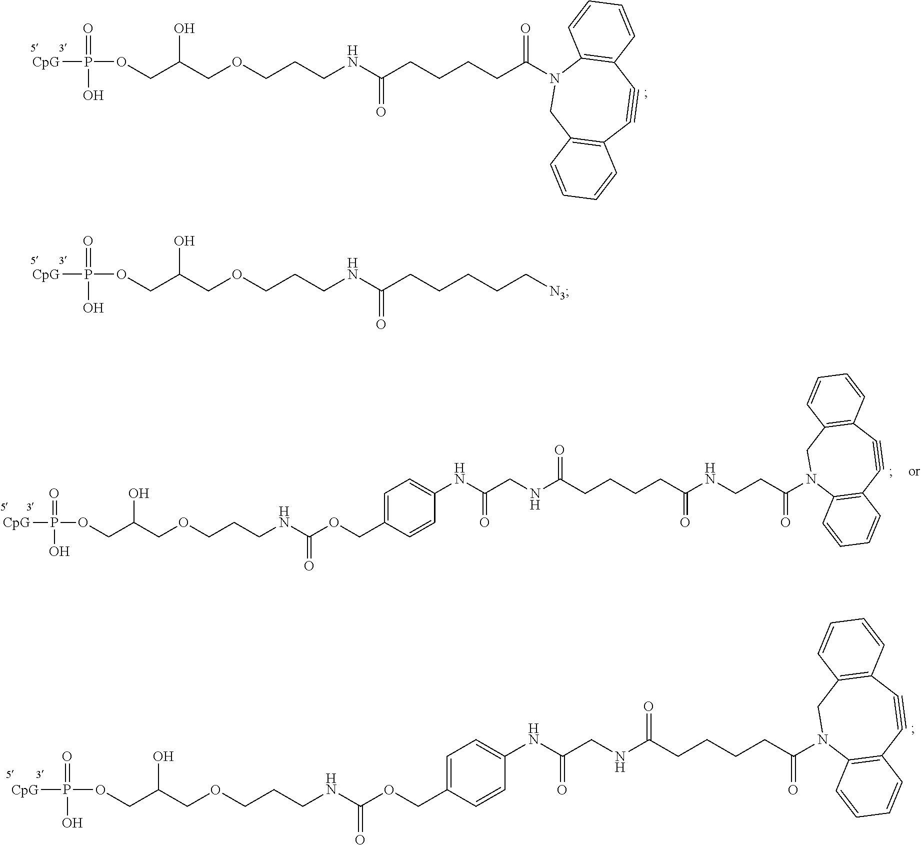

wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide; or

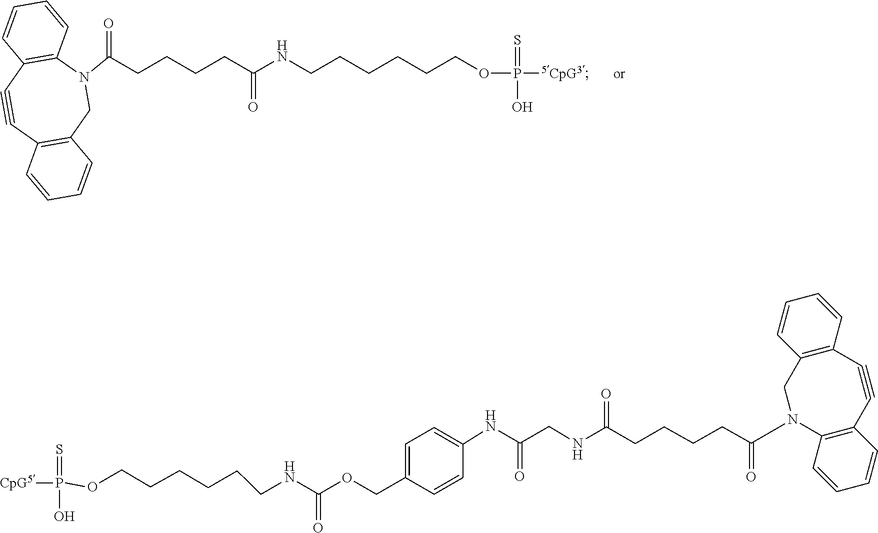

wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ (SD-101), connecting at 5′-O of the terminal nucleotide.

In certain embodiments of the present disclosure, a compound of formula (VII) is provided:

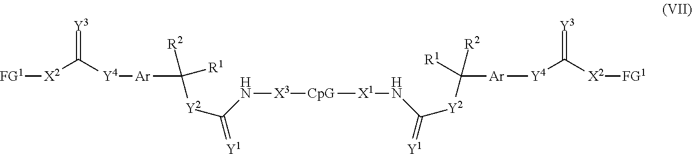

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X¹, X² and X³ are each independently a spacer moiety;
each R¹ and R² is, independently, a hydrogen, substituted or unsubstituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
Y¹ is O, NH or S;
Y² is O, NH or S;
Y³ is O, NH or S;
Y⁴ is O, NH or S;
FG¹ is a functional group capable of reacting through click chemistry; and
CpG is a TLR9 agonist oligodeoxynucleotide wherein either X¹ is covalently bound to 5′-O of the terminal nucleotide of CpG and X³ is covalently bound to 3′-O of the terminal nucleotide of CpG; or X¹ is covalently bound to 3′-O of the terminal nucleotide of CpG and X³ is covalently bound to 5′-O of the terminal nucleotide of CpG.

In certain embodiments, of the compound of formula (VII), R¹ and R², together with the atom to which they are attached, can join together to form a 3-8 membered ring that can optionally contain one or more O, NR^{XA} (wherein R^{XA} is alkyl, —C(O)-alkyl, or —S(O)₀₋₂-alkyl), or S(O)_{w} (wherein w is 0, 1, or 2).

In certain embodiments of the compound of formula (VII), the compound is a compound of formula (VII-A):

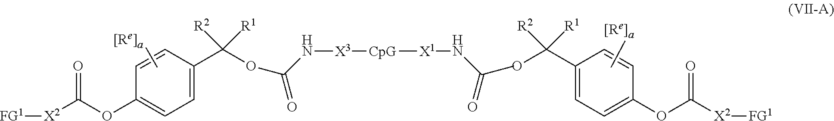

wherein:
X¹ and X³ are each independently selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; ** indicates the attachment point connecting to the amino group;
X² is selected from spacers C1 to C17 as described herein:
   wherein each oxygen is independently and optionally replaced by NH, NMe, NAc, S, or SO₂; * indicates the attachment point connecting to selected FG¹, ** indicates the attachment point connecting to the carbonyl group, and the spacer without * or ** indicates each of the two attachment points can be connected to either FG¹ or the carbonyl group;
each R¹ and R² is independently hydrogen, Me, or Et;
a is an integer of 0 to 2;
each R^{e} is independently selected from nitro, cyano, halogen, —OMe, —NHMe, —NHAc, —NHSO₂Me, and —OCF₃; and
FG¹ is a functional group capable of reacting through click chemistry and
CpG is a TLR9 agonist oligodeoxynucleotide wherein either X¹ is covalently bound to 5′-O of the terminal nucleotide of CpG and X³ is covalently bound to 3′-O of the terminal nucleotide of CpG; or X¹ is covalently bound to 3′-O of the terminal nucleotide of CpG and X³ is covalently bound to 5′-O of the terminal nucleotide of CpG.

In certain embodiments of the compound of formula (VII), The compound is a compound of formula (VII-B):

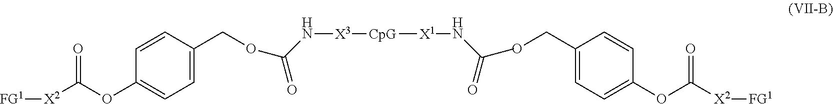

wherein:
X¹ and X³ are each independently selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; ** indicates the attachment point connecting to the amino group;
X² is selected from spacers C1, C3, C8, C10, C13, C18 and C19 as described herein:
   wherein * indicates the attachment point connecting to selected FG¹, ** indicates the attachment point connecting to the carbonyl group, and the spacer without * or ** indicates each of the two attachment points can be connected to either FG¹ or the carbonyl group;
FG¹ is an azide, dibenzocyclooctyne (DBCO), or alkynyl; and
CpG is a TLR9 agonist oligodeoxynucleotide wherein either X¹ is covalently bound to 5′-O of the terminal nucleotide of CpG and X³ is covalently bound to 3′-O of the terminal nucleotide of CpG; or X¹ is covalently bound to 3′-O of the terminal nucleotide of CpG and X³ is covalently bound to 5′-O of the terminal nucleotide of CpG.

In certain embodiments of the compound of formula (VII-B), X³ is **—C₃-C₁₂ alkylene-L¹-*; wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; and X¹ is spacer B3 as described herein: wherein L¹ is independently —OP(O)(OH)—, or —OP(S)(OH)—; and * indicates the attachment point connecting to a 3′-O of a terminal nucleotide of the CpG; and ** indicates the attachment point connecting to the amino group; X² is spacer C19 as described herein: wherein * indicates the attachment point connecting to selected FG¹, and ** indicates the attachment point connecting to the carbonyl group; FG¹ is dibenzocyclooctyne (DBCO). In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101); wherein X¹ is covalently bound to a 3′-O of a terminal nucleotide of CpG; and X³ is covalently bound to a 5′-O of a terminal nucleotide of CpG.

In certain embodiments, the present disclosure provides a compound of formula (VIII):

FG¹-X²-CpG-X¹-FG¹ (VIII)

or an enantiomer, a mixture of enantiomers, a diastereomer, a mixture of two or more diastereomers, a tautomer, a mixture of two or more tautomers, or an isotopic variant thereof;
wherein:
X¹ and X² are each independently a spacer moiety;
FG¹ is a functional group capable of reacting through click chemistry; and
CpG is a TLR9 agonist oligodeoxynucleotide wherein either X¹ is covalently bound to 3′-O of the terminal nucleotide of CpG and X² is covalently bound to 5′-O of the terminal nucleotide of CpG; or X¹ is covalently bound to 5′-O of the terminal nucleotide of CpG and X² is covalently bound to 3′-O of the terminal nucleotide of CpG.

In certain embodiments of the compound of formula (VIII), the X¹ and X² are each independently X³—NH—CO—X⁴—, wherein X³ is selected from **—C₃-C₁₂ alkylene-L¹-*; and spacers B1 to B5 as described herein:
wherein L¹ is independently a bond, —OP(O)(OH)—, or —OP(S)(OH)—; and * indicates the attachment point connecting to a 3′-O or 5′-O of a terminal nucleotide of the CpG; ** indicates the attachment point connecting to the amino group;
X⁴ is selected from spacers C1, C3, C8, C10, C13, C18 and C19 as described herein:
wherein * indicates the attachment point connecting to selected FG¹, ** indicates the attachment point connecting to the carbonyl group, and the spacer without * or ** indicates each of the two attachment points can be connected to either FG¹ or the carbonyl group.

In certain embodiments of the compound of formula (VIII), the X¹ and X² are each independently X³—NH—CO—X⁴—, wherein X³ is **—C₃-C₁₂ alkylene-L¹-* wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; or X³ is spacer B3 as described herein; wherein L 1 is independently —OP(O)(OH)—, or —OP(S)(OH)—; * indicates the attachment point connecting to a 3′-O of a terminal nucleotide of the CpG; and ** indicates the attachment point connecting to the amino group; and
X⁴ is selected from space C13 as described herein: wherein * indicates the attachment point connecting to selected FG¹, ** indicates the attachment point connecting to the carbonyl group; and
FG¹ is dibenzocyclooctyne (DBCO).

In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101).

In certain embodiments, the present disclosure provides a compound of formula (IX):

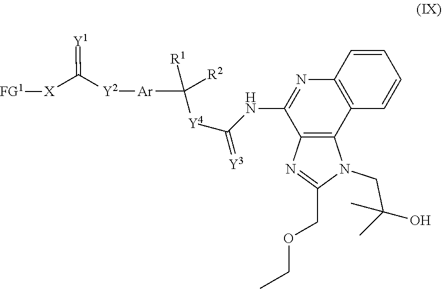

or an enantiomer, a mixture of enantiomers, a diastereomer, a mixture of two or more diastereomers, a tautomer, a mixture of two or more tautomers, or an isotopic variant thereof;
wherein:
Ar is substituted or unsubstituted aryl, or substituted or unsubstituted heteroaryl;
X is a spacer moiety;
each R¹ and R² is, independently, a hydrogen, substituted or unsubstituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
Y¹ is O or S;
Y² is O, NH or S;
Y³ is O or S;
Y⁴ is O or S; and
FG¹ is a functional group capable of reacting through click chemistry.

In certain embodiments, of the compound of formula (IX), R¹ and R², together with the atom to which they are attached, can join together to form a 3-8 membered ring that can optionally contain one or more O, NR^{XA} (wherein R^{XA} is alkyl, —C(O)-alkyl, or —S(O)₀₋₂-alkyl), or S(O)_{w} (wherein w is 0, 1, or 2).

In certain embodiments of the compound of formula (IX), the compound is a compound of formula (IX-A):

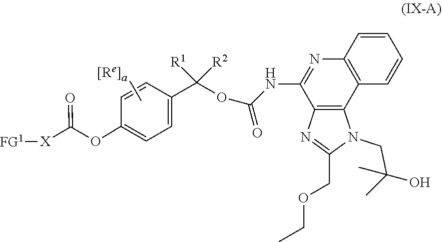

wherein:
X is selected from spacers C1 to C17 as described herein:
   wherein each oxygen is independently and optionally replaced by NH, NMe, NAc, S, or SO₂; * indicates the attachment point connecting to selected FG¹, ** indicates the attachment point connecting to the carbonyl group, and the spacer without * or ** indicates each of the two attachment points can be connected to either FG¹ or the carbonyl group;
each R¹ and R² is independently hydrogen, Me, or Et;
a is an integer of 0 to 2;
each R^{e} is independently selected from the group consisting of nitro, cyano, halogen, —OMe, —NHMe, —NHAc, —NHSO₂Me, and —OCF₃; and
FG¹ is a functional group capable of reacting through click chemistry.

In certain embodiments of the compound of formula (IX), the compound is a compound of formula (IX-B):

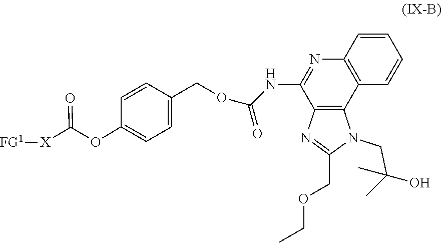

wherein:
X is selected from spacers C1, C3, C8, C10, C13, C18 and C19 as described herein
   wherein * indicates the attachment point connecting to selected FG¹, ** indicates the attachment point connecting to the carbonyl group, and the spacer without * or ** indicates each of the two attachment points can be connected to either FG¹ or the carbonyl group; and
FG¹ is an azide, dibenzocyclooctyne (DBCO), or alkynyl.

In certain embodiments of the compound of formula (IX-B), X is selected from spacers C1, C3, C13, and C18 as described herein: wherein * indicates the attachment point connecting to selected FG¹, ** indicates the attachment point connecting to the carbonyl group, and the spacer without * or ** indicates each of the two attachment points can be connected to either FG¹ or the carbonyl group; and FG¹ is an azide, or dibenzocyclooctyne (DBCO).

In certain embodiments of the compound of formula (IX), (IX-A), or (IX-B), X is C3, C13, or C18. In embodiments, X is C3 and n is 3. In embodiments, X is C13 and p is 4. In embodiments, X is C18; q is 3, and r is 2.

In certain embodiments of the compound of formula (IX), (IX-A), or (IX-B), the compound has following structure:

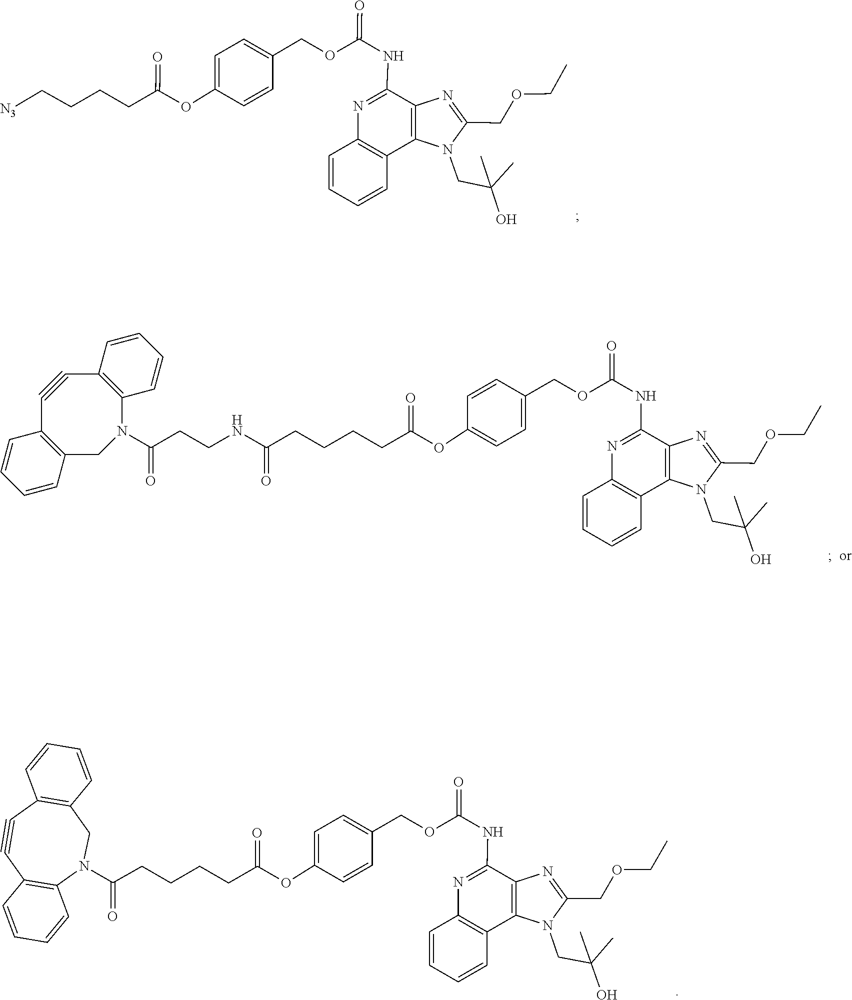 [SMILES: CCOCc1nc2c(NC(=O)OCc3ccc(OC(=O)CCCCN=[N+]=[N-])cc3)nc3ccccc3c2n1CC(C)(C)O ; CCOCc1nc2c(NC(=O)OCc3ccc(OC(=O)CCCCC(=O)NCCC(=O)N4Cc5ccccc5C#Cc5ccccc54)cc3)nc3ccccc3c2n1CC(C)(C)O ; CCOCc1nc2c(NC(=O)OCc3ccc(OC(=O)CCCCC(=O)N4Cc5ccccc5C#Cc5ccccc54)cc3)nc3ccccc3c2n1CC(C)(C)O]

In certain embodiments of formulae (II), (II-A), (II-B), (II-C), (III), (III-A), (III-B), (III-C), (IV), (IV-A), (IV-B), (V), (V-A), (V-B), (VI), (VII), (VII-A), (VII-B), (VIII), (IX), (IX-A), (IX-B), (XXII), (XXIII), (XXIII-A), and (XXIII-B), FG¹ is an azide, an alkynyl, or a cycloalkynyl group. In certain embodiments, the cycloalkynyl group is dibenzocyclooctyne (DBCO), or bicyclo[6.1.0]nonyne (BCN).

In certain embodiments, the present disclosure provides a conjugate of formula (X):

[A²-Z²-T-Z³]_{b2}-A¹-[Z¹-T-Z²-A²]_{b1} (X)

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
b1 is an integer of 0 or 1;
b2 is an integer of 0 or 1;
with the proviso that b1+b2 is 1 or 2;
each T is independently a triazole functional group;
Z¹, Z², and Z³ are each independently a spacer; and
A¹ and A² are each independently a therapeutic agent or an active moiety of a therapeutic agent; or a compound that decomposes to a therapeutic agent.

In certain embodiments of the conjugate of formula (X), one or more atoms or chemical groups in each therapeutic agent or compound that decomposes to a therapeutic agent is independently replaced to form a covalent bond to a spacer. In certain embodiments, one or more atoms in each therapeutic agent or compound that decomposes to a therapeutic agent is replaced with a covalent bond to a spacer. In certain embodiments, one or more chemical groups in each therapeutic agent or compound that decomposes to a therapeutic agent is replaced with a covalent bond to a spacer. In certain embodiments, one or more atoms in each therapeutic agent or compound that decomposes to a therapeutic agent is replaced with a chemical group linking the therapeutic agent or compound to a spacer. In certain embodiments, one or more chemical groups in each therapeutic agent or compound that decomposes to a therapeutic agent is replaced with a chemical group linking the therapeutic agent or compound to a spacer. In certain embodiments, one or more hydrogens (e.g., C—H, N—H, O—H, or S—H), in each therapeutic agent or compound that decomposes to a therapeutic agent is replaced to form the covalent bond to a spacer. In certain embodiments, A¹ and A² are each independently a STING agonist, a TLR9 agonist, or a TLR7/8 agonist. In certain embodiments, A¹ is a TLR9 agonist and A² is a TLR7/8 agonist. In certain embodiments, A¹ is a TLR9 agonist and A² is a STING agonist.

In certain embodiments, A² is a STING agonist. In certain embodiments, one or more hydrogens in the STING agonist (e.g., one or more hydrogens in a S—H moiety) is replaced with a covalent bond to the spacer. In certain embodiments, the STING agonist is a cyclic dinucleotide (CDN).

In certain embodiments, A² is a TLR7/8 agonist or derivative thereof. In certain embodiments, A is a TRL7/8 agonist or derivative thereof. In certain embodiments, the TLR7/8 agonist is R848.

In certain embodiments, a hydrogen (e.g., N—H) in the TLR7/8 agonist (e.g., R848) is replaced with a *—C(O)-O—** group linking the TLR7/8 agonist to a spacer; wherein * indicates the point of attachment to the TLR7/8 agonist ** indicates the point of attachment to the spacer. In certain embodiments, a hydrogen (e.g., N—H) in the TLR7/8 agonist (e.g., R848) is replaced with a covalent bond to the spacer.

In certain embodiments of the conjugate of formula (X),
Z¹ is

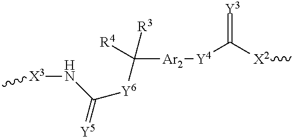

wherein Z¹ is connected to A¹ through X³ and T through X²;
Z² is

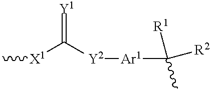

wherein Z² is connected to T through X¹; and
Z³ is

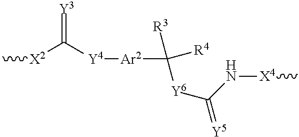

wherein Z³ is connected to A¹ through X⁴ and T through X².

In certain embodiments, the conjugate of formula (X) is a conjugate of formula (X-A)

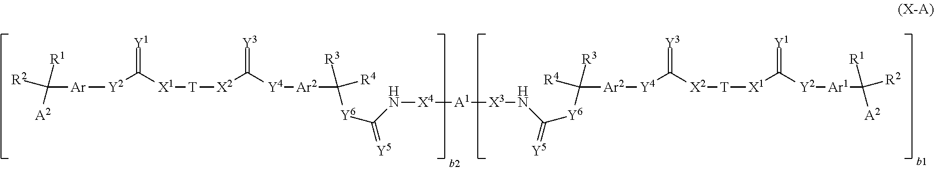

wherein:
b1 is an integer of 0 or 1;
b2 is an integer of 0 or 1;
with the proviso that b1+b2 is 1 or 2;
each T is independently a triazole functional group;
Ar¹ and Ar² are each independently a substituted or unsubstituted aryl or heteroaryl;
X¹, X², X³, and X⁴ are each independently a spacer;
R¹, R², R³ and R⁴ are each independently a hydrogen, alkyl, substituted alkyl, substituted or unsubstituted alkenyl, substituted or unsubstituted alkynyl, aryl, or substituted aryl;
R¹ and R², or R³ and R⁴ together with the atom to which they are attached, can join together to form a substituted or unsubstituted 3-8 membered ring that can optionally contain one or two heteroatoms;
Y¹, Y², Y³, Y⁴, Y⁵ and Y⁶ are each independently O, NH or S; and
A¹ and A² are each independently a therapeutic agent or an active moiety of a therapeutic agent; or a compound that decomposes to a therapeutic agent.

In certain embodiments, the conjugate of formula (X) has a structure according to formula (X-B):

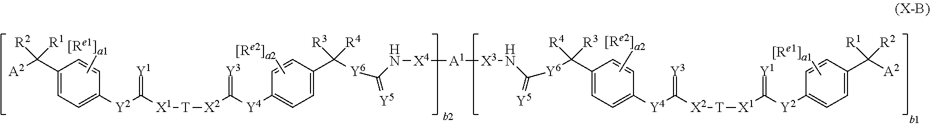

wherein:
b1 is an integer of 0 or 1;
b2 is an integer of 0 or 1;
with the proviso that b1+b2 is 1 or 2;
a1 and a2 are each independently an integer of 0 to 4;
each R^{e1} and R^{e2} are each independently selected from nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, substituted or unsubstituted alkenyl, substituted or unsubstituted alkynyl, aryl or heteroaryl, and substituted aryl or heteroaryl;
each T is independently a triazole functional group;
X¹, X² are selected from spacers C1 to C17 or C20 to C22 as described herein, each oxygen is independently and optionally replaced by NH, NMe, NAc, S, or SO₂; * indicates the attachment point connecting to T, ** indicates the attachment point connecting to the carbonyl or thiocarbonyl group, and the spacer without * or ** indicates each of the two attachment points can be connected to either T or the carbonyl or thiocarbonyl group;
X³ and X⁴ are each independently selected from **—C₃-C₁₂ alkylene-L¹-*; and spacers B1 to B5 as described herein:
   wherein L¹ is independently a bond, —OP(O)(OH)—, or —OP(S)(OH)—; * indicates the attachment point connecting to A¹; and ** indicates the attachment point connecting to the amino group;
R¹, R², R³ and R⁴ are each independently hydrogen or a C₁-C₆ alkyl; or
R¹ and R² together with the carbon atom to which they are attached, can join together to form a 3-6 membered ring that can optionally contain one or more of O, NMe, NAc, NSO₂Me, S, or SO₂;
R³ and R⁴ together with the carbon atom to which they are attached, can join together to form a 3-6 membered ring that can optionally contain one or more of O, NMe, NAc, NSO₂Me, S, or SO₂;
Y¹, Y², Y³, Y⁴, Y⁵ and Y⁶ are each independently O, NH or S; and
A¹ and A² are each independently a therapeutic agent or an active moiety of a therapeutic agent; or a compound that decomposes to a therapeutic agent.

In certain embodiments, the conjugate of formula (X) has a structure according to formula (X-C):

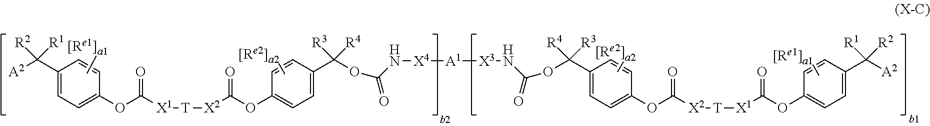

wherein:
b1 is an integer of 0 or 1;
b2 is an integer of 0 or 1;
with the proviso that b1+b2 is 1 or 2;
a1 and a2 are each independently an integer of 0 to 2;
each R^{e1} and R^{e2} is independently selected from nitro, cyano, halogen, —OMe, —NHMe, —NHAc, —NHSO₂Me, and —OCF₃;
each T is independently a triazole functional group;
X¹, X² are selected from spacers C1 to C17 as described herein, each oxygen is independently and optionally replaced by NH, NMe, NAc, S, or SO₂; * indicates the attachment point connecting to T, ** indicates the attachment point connecting to the carbonyl group; and the spacer without * or ** indicates the attachment points can be connected to either T or the carbonyl group.
X³ and X⁴ are each independently selected from **—C₃-C₁₂ alkylene-L¹-*; and spacers B1 to B5 as described herein:
wherein L¹ is independently a bond, —OP(O)(OH)—, or —OP(S)(OH)—; * indicates the attachment point connecting to A¹, and ** indicates the attachment point connecting to the amino group;
R¹, R², R³ and R⁴ are each independently hydrogen, Me, or Et; and
A¹ and A² are each independently a therapeutic agent or an active moiety of a therapeutic agent; or a compound that decomposes to a therapeutic agent.

In certain embodiments, the conjugate of formula (X) has a structure according to formula (X-D):

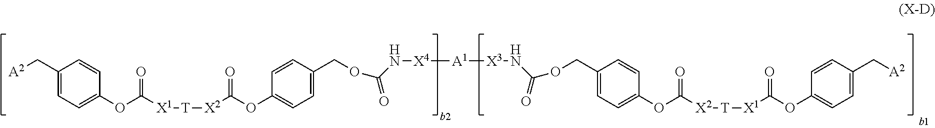

wherein:
b1 is an integer of 0 or 1;
b2 is an integer of 0 or 1;
with the proviso that b1+b2 is 1 or 2;
each T is independently a triazole functional group;
X¹, X² are each independently selected from spacers C1, C3, C8, C10, C13, C18 and C19 as described herein:

wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
X³ and X⁴ are each independently selected from **—C₃-C₁₂ alkylene-L¹-* and spacer B3 as described herein: wherein L¹ is independently —OP(O)(OH)—, or —OP(S)(OH)—; * indicates the attachment point connecting to A¹; and ** indicates the attachment point connecting to the amino group; and
A¹ and A² are each independently a therapeutic agent or an active moiety of a therapeutic agent; or a compound that decomposes to a therapeutic agent.

In certain embodiments, the conjugate of formula (X) has a structure according to formula (X-E):

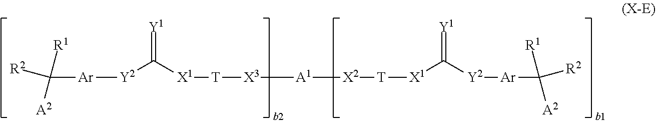

wherein:
b1 is an integer of 0 or 1;
b2 is an integer of 0 or 1;
with the proviso that b1+b2 is 1 or 2;
each T is independently a triazole functional group;
Ar is a substituted or unsubstituted aryl, or substituted or unsubstituted heteroaryl;
X¹, X², and X³ are each independently a spacer;
R¹ and R² are each independently a hydrogen, alkyl, substituted alkyl, substituted or unsubstituted alkenyl, substituted or unsubstituted alkynyl, aryl, or substituted aryl; or
R¹ and R² together with the atom to which they attach can join together to form a 3-8 membered ring that can optionally contain one or two heteroatoms;
Y¹ and Y² are each independently O, NH or S; and
A¹ and A² are each independently a therapeutic agent or an active moiety of a therapeutic agent; or a compound that decomposes to a therapeutic agent.

In certain embodiments, the conjugate of formula (X) has a structure according to formula (X-F):

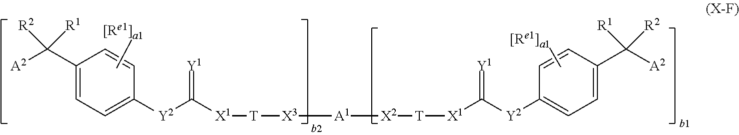

wherein:
b1 is an integer of 0 or 1;
b2 is an integer of 0 or 1;
with the proviso that b1+b2 is 1 or 2;
a1 and a2 are each independently an integer of 0 to 4;
each R^{e1} is independently nitro, cyano, halogen, amide, substituted amide, sulfone, substituted sulfone, sulfonamide, substituted sulfonamide, alkoxy, substituted alkoxy, alkyl or cycloalkyl, substituted alkyl or cycloalkyl, substituted or unsubstituted alkenyl, substituted or unsubstituted alkynyl, aryl or heteroaryl, and substituted aryl or heteroaryl;
each T is independently a triazole functional group;
X¹ is selected from spacers C1 to C17 or C20 to C22 as described herein, each oxygen is independently and optionally replaced by NH, NMe, NAc, S, or SO₂; * indicates the attachment point connecting to T, ** indicates the attachment point connecting to the carbonyl or thiocarbonyl group, and the spacer without * or ** indicates each of the two attachment points can be connected to either T or the carbonyl or thiocarbonyl group;
X² and X³ are each independently —X⁴—NH—CO—X⁵—, wherein X⁴ is selected from **—C₃-C₁₂ alkylene-L¹-*; and spacers B1 to B7 as described herein: wherein L¹ is independently a bond, —OP(O)(OH)—, or —OP(S)(OH)—; * indicates the attachment point connecting to A¹; and ** indicates the attachment point connecting to the amino group;
X⁵ is selected from spacers C1, C3, C8, C10, C13, C18 and C19 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
R¹ and R² are each independently hydrogen or a C₁-C₆ alkyl; or
R¹ and R² together with the atom to which they are attached, can join together to form a 3-6 membered ring that can optionally contain one or more of O, NMe, NAc, NSO₂Me, S, or SO₂;
Y¹ and Y² are each independently O, NH or S; and
A¹ and A² are each independently a therapeutic agent or an active moiety of a therapeutic agent; or a compound that decomposes to a therapeutic agent.

In certain embodiments, the conjugate of formula (X) has a structure according to formula (X-G):

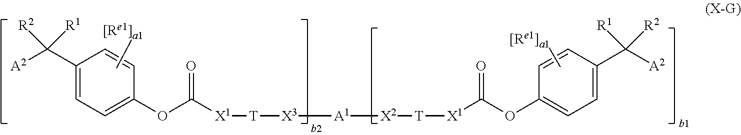

wherein:
b1 is an integer of 0 or 1;
b2 is an integer of 0 or 1;
with the proviso that b1+b2 is 1 or 2;
a1 is an integer of 0 to 2;
each R^{e1} and Rₑ₂ is independently selected from nitro, cyano, halogen, —OMe, —NHMe, —NHAc, —NHSO₂Me, and —OCF₃;
each T is independently a triazole functional group;
X¹ is selected from spacers C1 to C17 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
X² and X³ are each independently —X⁴—NH—CO—X⁵—, wherein X⁴ is selected from **—C₃-C₁₂ alkylene-L¹-*; and spacers B1 to B7 as described herein: wherein L¹ is independently a bond, —OP(O)(OH)—, or —OP(S)(OH)—; * indicates the attachment point connecting to A¹; and ** indicates the attachment point connecting to the amino group;
X⁵ is selected from spacers C1, C3, C8, C10, C13, C18 and C19 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
R¹ and R² are each independently hydrogen, Me, or Et; and
A¹ and A² are each independently a therapeutic agent or an active moiety of a therapeutic agent; or a compound that decomposes to a therapeutic agent.

In certain embodiments, the conjugate of formula (X) has a structure according to formula (X-H):

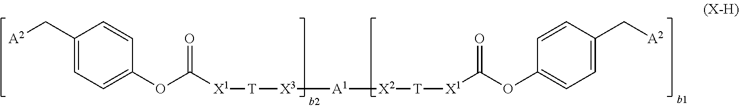

wherein:
b1 is an integer of 0 or 1;
b2 is an integer of 0 or 1;
with the proviso that b1+b2 is 1 or 2;
each T is independently a triazole functional group;
X¹ is selected from spacers C1, C3, C8, C10, C13, C18 and C19 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
X² and X³ are each independently —X⁴—NH—CO—X⁵—, wherein X⁴ is selected from **—C₃-C₁₂ alkylene-L^{1-*}; spacers B3, B6 or B7 as described herein: wherein L¹ is independently —OP(O)(OH)—, or —OP(S)(OH)—; * indicates the attachment point connecting to A¹; and ** indicates the attachment point connecting to the amino group;
X⁵ is selected from spacers C13 and C18 as described herein:
   wherein * indicates the attachment point connecting to T, ** indicates the attachment point connecting to the carbonyl group; and
A¹ and A² are each independently a therapeutic agent or an active moiety of a therapeutic agent; or a compound that decomposes to a therapeutic agent.

In certain embodiments of the conjugates of the present disclosure, X⁴ is B3 or B6 and L¹ is —OP(O)(OH)—.

In certain embodiments of the conjugates of the present disclosure, X⁴ is **—C₃-C₁₂ alkylene-L¹-*; and L¹ is —OP(S)(OH)—.

In some of the conjugates of the present disclosure, X⁵ is C13 or C18. In certain embodiments, X⁵ is C13. In certain embodiments, X⁵ is C18.

In certain embodiments of the conjugate of formula (X), the therapeutic agent is a STING agonist, a TLR9 agonist, or a TLR7/8 agonist.

In certain embodiments of the conjugates of formula (X), the conjugate comprises a STING agonist described in OncoImmunology, 9:1, 1777624; Theranostics. 2019; 9(25): 7759-7771; US20140341976; WO2016145102; WO2019232392; WO2017027645; WO2017027646; WO2017123657; WO2017123669; WO2018098203; WO2017161349; WO2018009652; WO2019069275; or WO2019069270; the contents of which are hereby incorporated by reference.

In certain embodiments of the conjugate of formula (X), the conjugate comprises a TLR9 agonist described in Nat Rev Drug Discov 2010 April; 9(4):293-307; Front Immunol 2019 Oct. 22; 10:2388; Immunotherapy 2009 November; 1(6):949-64; Oncogene 2008 Jan. 7; 27(2):161-7; J Clin Invest 2007 May; 117(5):1184-94; J Leukoc Biol 2013 June; 93(6):847-63; WO 2004/058179 A1; WO 2018/053242 A1; WO1998018810A1; WO2003024480A2; WO2015013673A1; and WO2017050806A1, the contents of which are hereby incorporated by reference.

In certain embodiments of the conjugate of formula (X), A¹ is a TLR9 agonist. In certain embodiments of the conjugate of formula (X), A² is a TLR7/8 agonist or derivative thereof. In certain embodiments of the conjugate of formula (X), A² is a STING agonist. In certain embodiments of the conjugate of formula (X), A¹ is a TLR9 agonist and A² is a STING agonist. In certain embodiments of the conjugate of formula (X), A¹ is a TLR9 agonist and A² is a TLR7/8 agonist or derivative thereof. In certain embodiments of the conjugate of formula (X), the STING agonist is a CDN. In certain embodiments of the conjugate of formula (X), the TLR7/8 agonist is R848 or a derivative thereof. In certain embodiments of the conjugate of formula (X), A¹ is CpG that is a TLR9 agonists CpG ODNs (oligodeoxynucleotides) that is short synthetic single-stranded DNA molecules containing unmethylated CpG motifs.

In certain embodiments, CpG comprises a following formula:

X₁X₂CGX₃X₄3′
wherein X₁, X₂, X₃, and X₄ are any nucleotide, and
CpG is connecting with spacer at 5′-O or/and 3′-O of the terminal nucleotide.

In certain embodiments, CpG is: 5′-GGGGGGGGGGGACGATCGTCGGGGGGGGGG-3′ (CMP-001); connecting at 5′-O or/and 3′-O of the terminal nucleotide.

In certain embodiments, CpG comprises a following formula:

5′N₁X₁CGX₂N₂3′
wherein at least one nucleotide separates consecutive CpGs; X₁ is adenine, guanine, or thymine; X₂ is cytosine or thymine; N is any nucleotide and N₁+N₂ is from about 0-26 bases with the proviso that N₁ and N₂ does not contain a CCGG quadmer or more than one CCG or CGG trimer; the nucleic acid sequence is from about 8-30 bases in length; and
CpG is connecting with spacer at 5′-O or/and 3′-O of the terminal nucleotide.

In certain embodiments, CpG is a phosphorothioate linked 5′-T*C*G*T*C*G*T*T*T*T*G*T*C*G*T*T*T*T*G*T*C*G*T*T-3′ (PF-3512676); connecting at 5′-O or/and 3′-O of the terminal nucleotide.

In certain embodiments, CpG comprises the following formula:

5′-Nₓ(TCG(N_{q}))_{y}N_{w}(X₁X₂CGX₂′X₁′(CG)ₚ)_{z} a)
wherein N are nucleosides, x=0, y=1, w=0, p=0 or 1, q=0, 1 or 2, and z=1-20, X₁ and X₁′ are self-complimentary nucleosides, X₂ and X₂′ are self-complimentary nucleosides, and wherein the 5′ T of the (TCG(N_{q}))_{y} sequence is positioned at the 5′ end of the polynucleotide; and
b) a palindromic sequence at least 8 bases in length wherein the palindromic sequence comprises the first (X₁X₂CGX₂′X₁′) of the (X₁X₂CGX₂′X₁′(CG)ₚ)_{z} sequences, wherein the polynucleotide is at least 15 bases in length; and
CpG is connecting with spacer at 5′-O or/and 3′-O of the terminal nucleotide.

In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101); connecting at 5′-O or/and 3′-O of the terminal nucleotide.

In certain embodiments, CpG comprises at least two oligonucleotides linked together at their 3′ ends, an internucleotide linkage, or a functionalized nucleobase or sugar by a non-nucleotidic linker, wherein at least one of the oligonucleotides is an immunostimulatory oligonucleotide having an accessible 5′ end and comprising an immunostimulatory dinucleotide selected from the group consisting of CG, C^{#}G, CG^{#}, and C^{#}G^{#}, wherein C is cytidine or 2′-deoxycytidine, C^{#} is 2′-deoxythymidine, arabinocytidine, 2′-deoxy-2′-substituted arabinocytidine, 2′-O-substituted arabinocytidine, 2′-deoxy-5-hydroxycytidine, 2′-deoxy-N4-alkyl-cytidine, 2′-deoxy-4-thiouridine or other non-natural pyrimidine nucleoside, G is guanosine or 2′-deoxyguanosine, G^{#} is 2′-deoxy-7-deazaguanosine, 2′-deoxy-6-thioguanosine, arabinoguanosine, 2′-deoxy-2′ substituted-arabinoguanosine, 2′-O-substituted-arabinoguanosine, or other non-natural purine nucleoside, p is an internucleotide linkage selected from the group consisting of phosphodiester, phosphorothioate, and phosphorodithioate; and
CpG is connecting with spacer at one or two 5′-O of the terminal nucleotide or/and internucleotide linkage.

In certain embodiments, CpG is a phosphorothioate linked 5′-T*C*G₁*A*A*C*G₁*T*T*C*G₁*-X-*G₁*C*T*T*G₁*C*A*A*G₁*C*T*-5′, wherein X is a glycerol linker and GI is 2′-deoxy-7-deazaguanosine (IMO-2125); connecting at one or two of the terminal nucleotide or/and glycerol.

In certain embodiments of the compound of formula (X), (X-A), (X-B), (X-C), (X-D), (X-E), (X-F), (X-G), or (X-H), A¹ is a CpG and *indicates the attachment point connecting to A¹ via i) 5′-O of the terminal nucleotide of CpG; and/or ii) 3′-O of the terminal nucleotide of CpG.

In certain embodiments of formula (X), (X-A), (X-B), (X-C), (X-D), (X-E), (X-F), (X-G), or (X-H), A¹ is a CpG with following sequences: a phosphorothioate linked 5′-T*C*G *A*A*C *G*T*T *C*G*A *A*C*G *T*T*C *G*A*A *C*G*T *T*C*G *A*A*T-3′ (SD-101); or a phosphorothioate linked 5′-T*C*G₁*A*A*C*G₁*T*T*C*G₁*-X-*G₁*C*T*T*G₁*C*A*A*G₁*C*T*-5′, wherein X is a glycerol linker and GI is 2′-deoxy-7-deazaguanosine (IMO-2125); or 5′-GGGGGGGGGGGACGATCGTCGGGGGGGGGG-3′ (CMP-001) or phosphorothioate linked 5′-T*C*G*T*C*G*T*T*T*T*G*T*C*G*T*T*T*T*G*T*C*G*T*T-3′ (PF-3512676), connecting through 5′-O or/and 3′-O of the terminal nucleotide of CpG.

In certain embodiments of the conjugate of formula (X), the conjugate has a structure according to formula (X-1):

[A²-Z²-T-Z³]_{b2}-CpG-[Z¹-T-Z²-A²]_{b1} (X-1)
wherein:
CpG is a TLR9 agonist oligodeoxynucleotide;
A² is a STING agonist;
Z¹ and Z³ are each independently selected from the group consisting of:

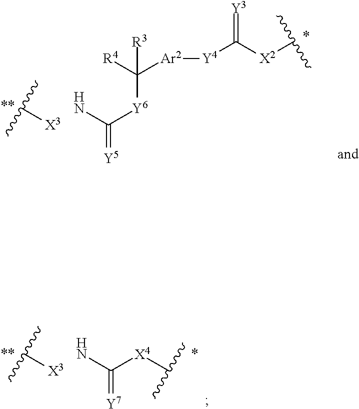

wherein * indicates the attachment point to T and ** indicates the attachment point to a 3′-O or 5′-O of a terminal nucleotide of the CpG;
Z² is selected from the group consisting of:

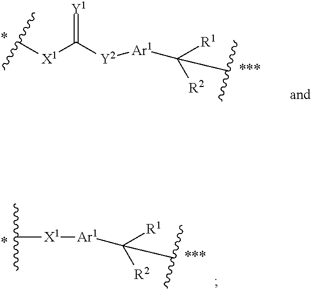

wherein * indicates the attachment point to T and *** indicates the attachment point to the STING agonist;
Ar¹ and Ar² are each independently a substituted or unsubstituted aryl or substituted or unsubstituted heteroaryl;
X¹, X², X³, and X⁴ are each independently a spacer;
R¹, R², R³ and R⁴ are each independently a hydrogen, substituted or unsubstituted alkyl, substituted or unsubstituted alkenyl, substituted or unsubstituted alkynyl, or substituted or unsubstituted aryl; or
R¹ and R², or R³ and R⁴ together with the atom to which they are attached, can join together to form a substituted or unsubstituted 3-8 membered ring that can optionally contain one or two heteroatoms; and
Y¹, Y², Y³, Y⁴, Y⁵, Y⁶, and Y₇ and are each independently O or S.

In certain embodiments of the conjugate of formula (X) and (X-1), Z¹-T-Z², and/or Z²-T-Z³ is selected from the group consisting of:

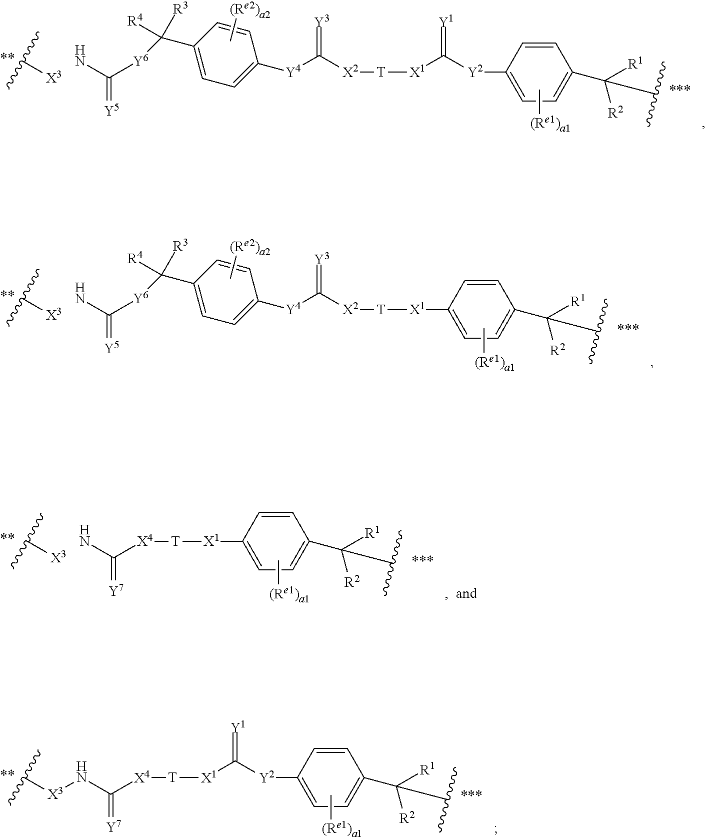

wherein ** indicates the attachment point to a 3′-O or 5′-O of a terminal nucleotide of the CpG and *** indicates the attachment point to the STING agonist;
wherein:
a1 and a2 are each independently an integer of 0 to 4; and
each R^{e1} and Rₑ₂ is independently for each occurrence selected from the group consisting of nitro, cyano, halogen, substituted or unsubstituted amide, substituted or unsubstituted sulfone, substituted or unsubstituted sulfonamide, substituted or unsubstituted alkoxy, substituted or unsubstituted alkyl, substituted or unsubstituted alkenyl, substituted or unsubstituted alkynyl, substituted or unsubstituted cycloalkyl, substituted or unsubstituted aryl, and substituted or unsubstituted heteroaryl.

In certain embodiments X¹, X² and X⁴ are selected from the group consisting of:

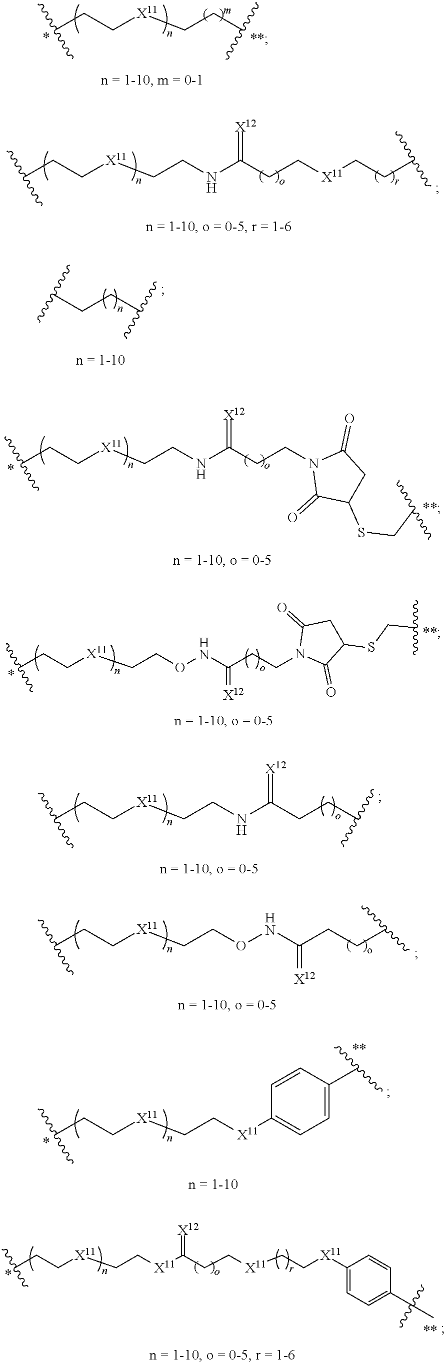

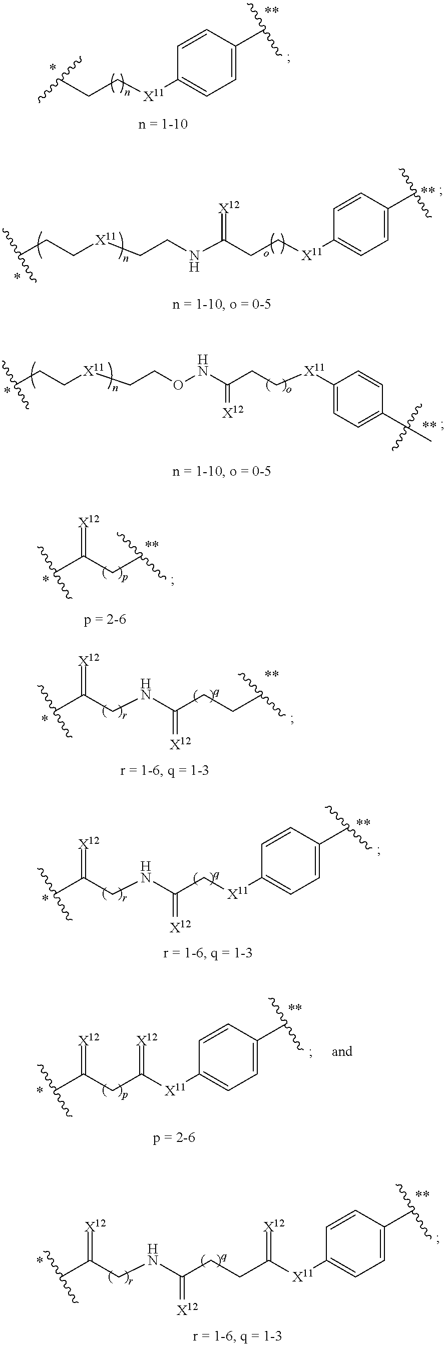

wherein
X¹¹ is independently selected from the group consisting of: —O—, —NC(O)R^{C}—, —NR^{C}—, —S—, SO₂, and CR^{D}R^{E};
X¹² is independently O, NR^{C}, or S;
R^{C} is hydrogen, substituted or unsubstituted alkyl, substituted or unsubstituted alkenyl, substituted or unsubstituted alkynyl, substituted or unsubstituted cycloalkyl, substituted or unsubstituted aryl or substituted or unsubstituted heteroaryl; and
R^{D} and R^{E} are each independently hydrogen, substituted or unsubstituted alkyl, substituted or unsubstituted alkenyl, substituted or unsubstituted alkynyl, substituted or unsubstituted cycloalkyl, substituted or unsubstituted aryl or substituted or unsubstituted heteroaryl;
wherein * indicates the attachment point connecting to T, and ** indicates the attachment point connecting to the carbonyl or thiocarbonyl group, and the spacer without * or ** indicates each of the two attachment points can be connected to either T or the carbonyl or thiocarbonyl group.

In certain embodiments of the conjugate of formula (X) and (X-1), X¹, X² and X⁴ are independently selected from the group consisting of:

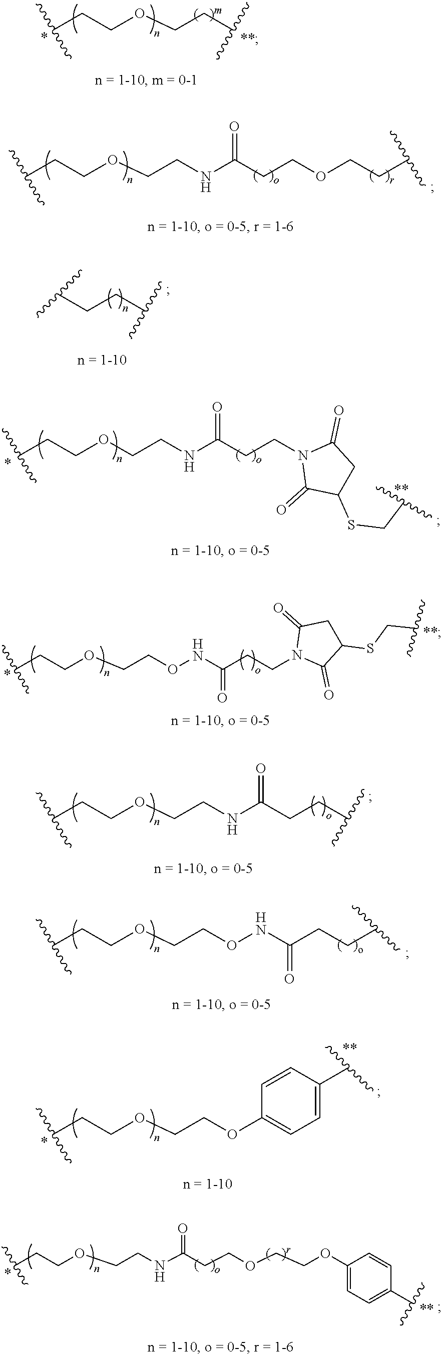

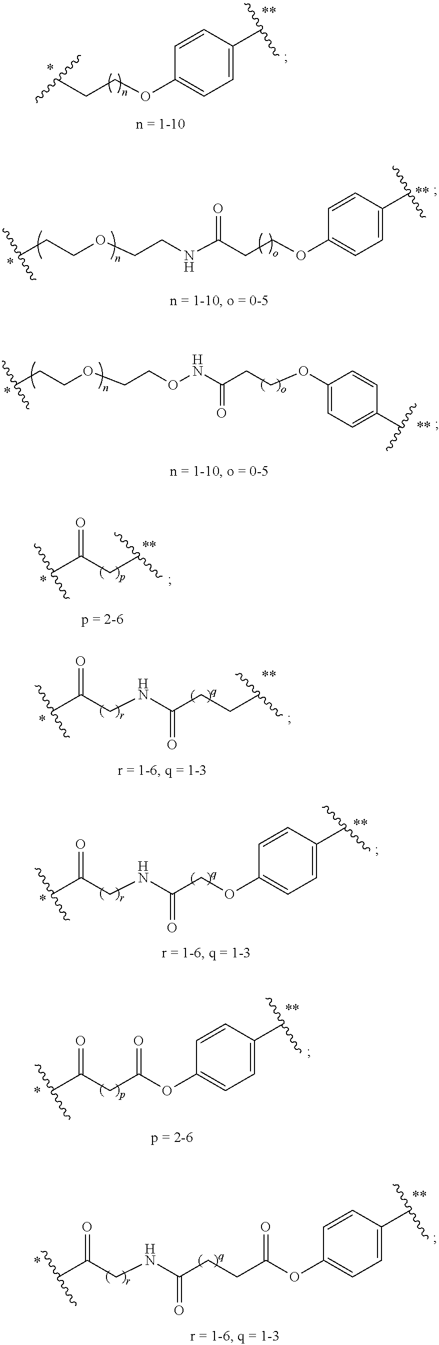

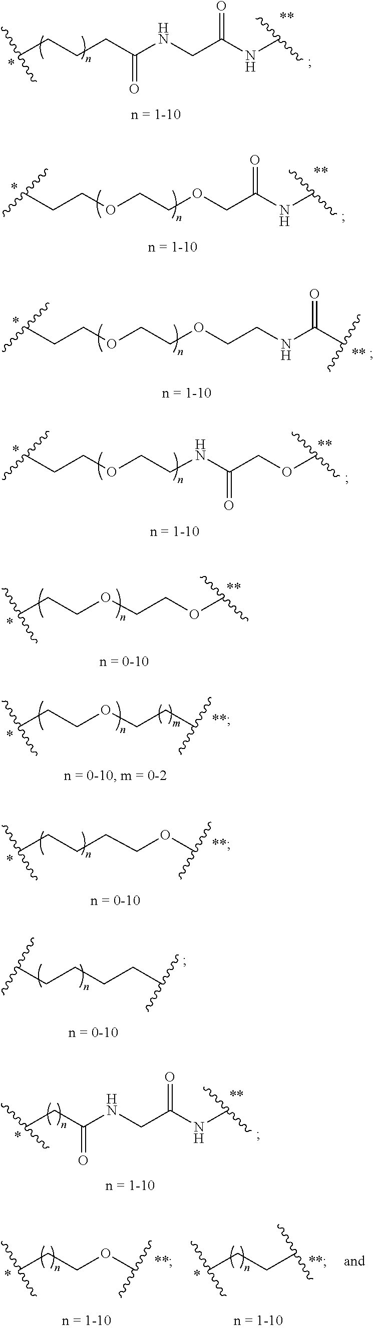

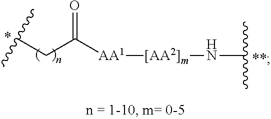

wherein AA¹-[AA²]ₘ comprises the groups selected from: Gly, Lys, Val-Ala, Val-Lys, Val-Cit, Ala-Lys, Phe-Lys, Phe-Cit, Leu-Cit, Ile-Cit, Trp-Cit, Phe-Arg, Phe-Leu, Ala-Phe, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Pro-(D)Lys, Met-Cit-Val, Gly-Cit-Val, Phe-Phe-Lys, (D)Phe-Phe-Lys, (D)Ala-Phe-Lys, Gly-Phe-Lys, Glu-Val-Cit, Ser-Val-Cit, Gly-Phe-Leu-Gly, and Ala-Leu-Ala-Leu.

In certain embodiments of the conjugate of formula (X) and (X-1), X¹, X² and X⁴ are independently selected from the group consisting of:

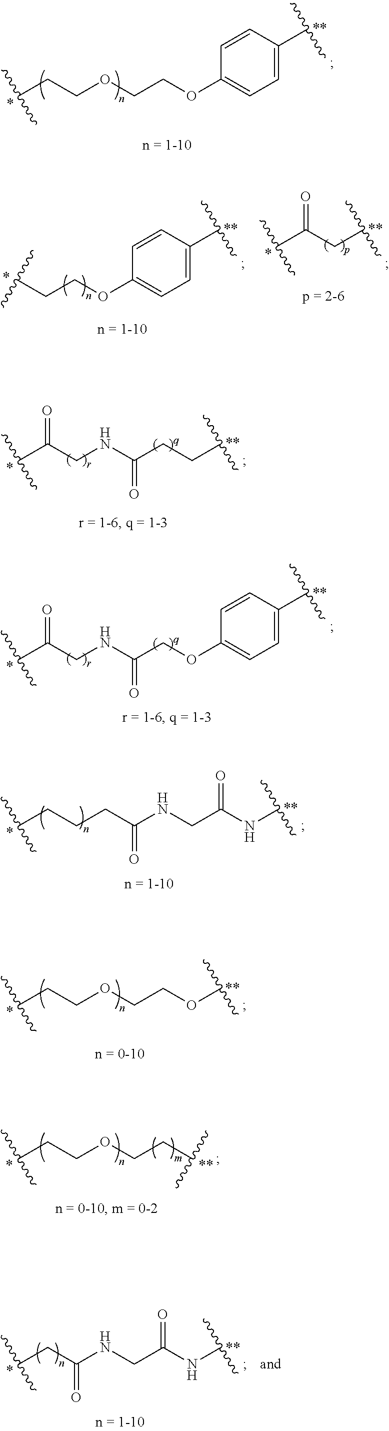

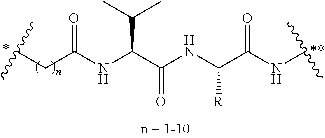

wherein R is methyl or

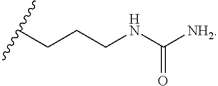 [SMILES: *CCCNC(N)=O]

In certain embodiments of the conjugate of formula (X) and (X-1), X¹, X² and X⁴ are each independently:

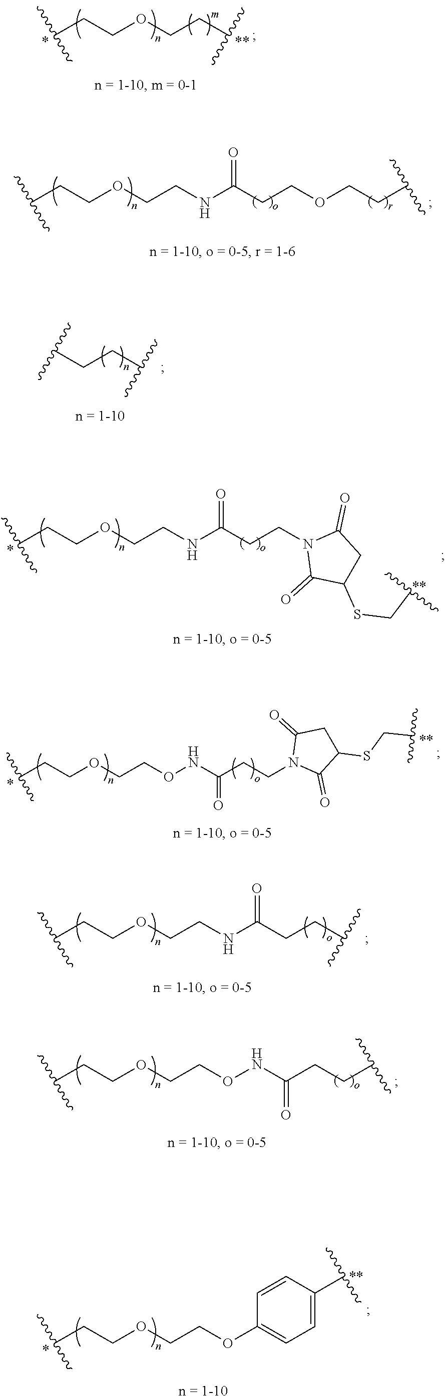

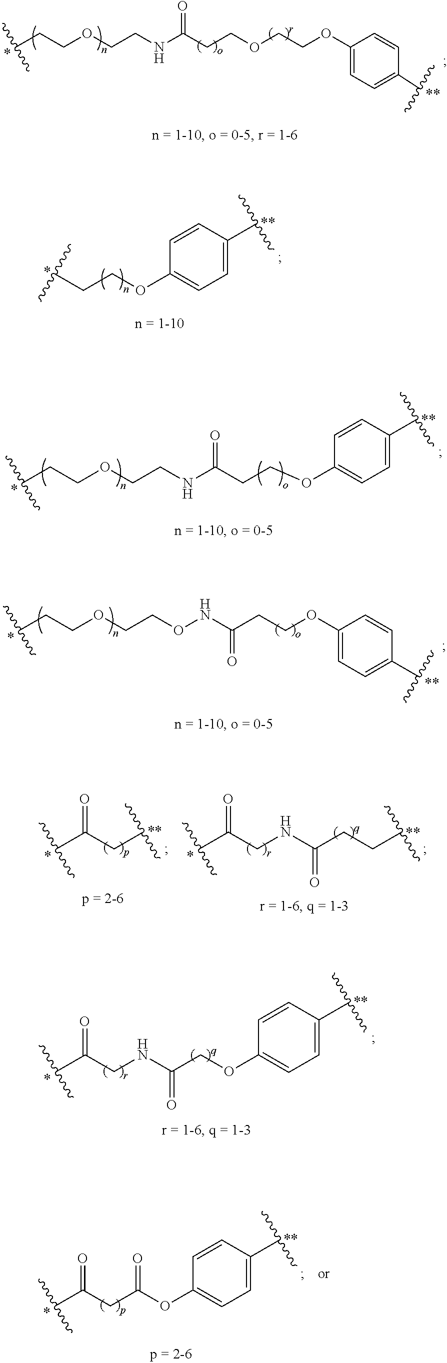

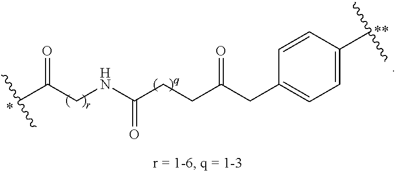

In certain embodiments of the conjugate of formula (X) and (X-1), X¹, X² and X⁴ are each independently:

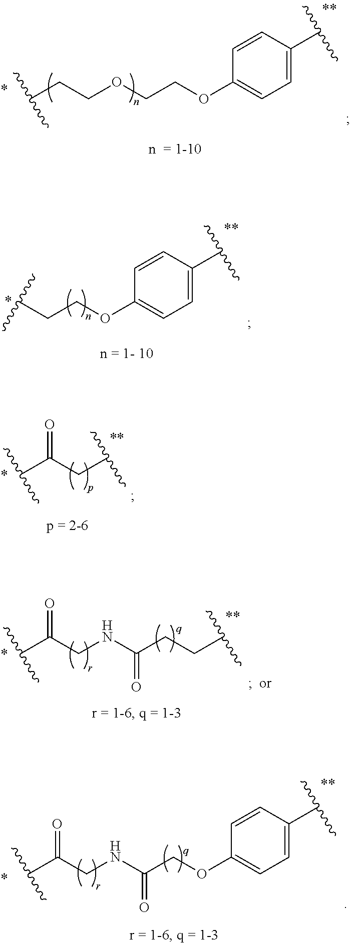

In certain embodiments of the conjugate of formula (X) and (X-1), X³ is independently: **—C₃-C₁₂ alkylene-L¹-*;

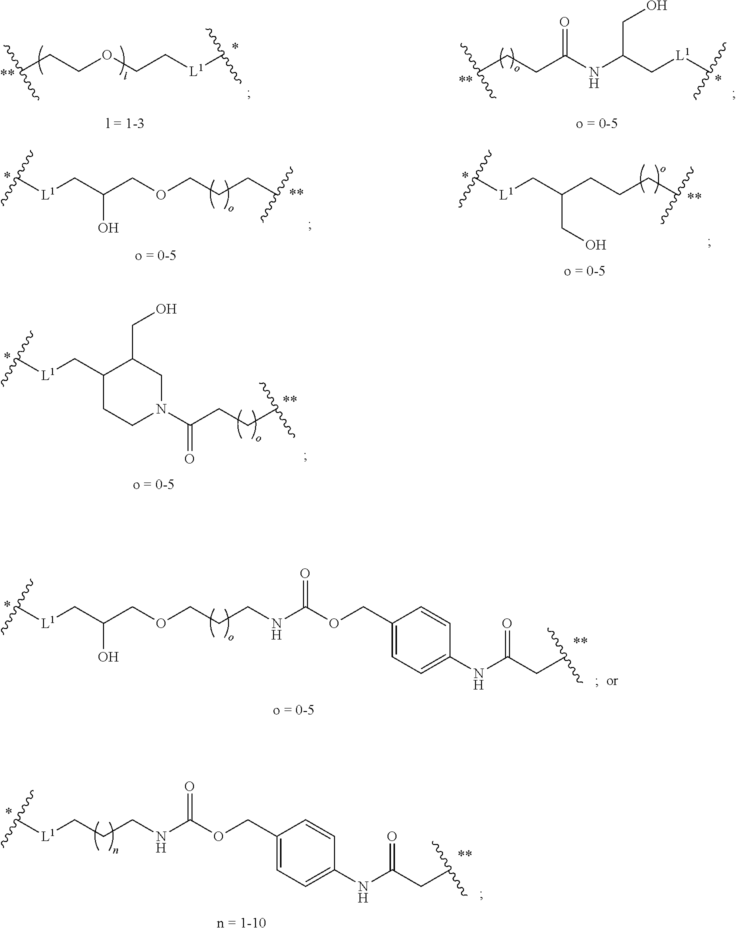

wherein L¹ is independently a bond, —OP(O)(OH)—, or —OP(S)(OH)—; * indicates the attachment point connecting to a 3′-O or 5′-0 of a terminal nucleotide of the CpG; ** indicates the attachment point connecting to the amino group. In certain embodiments, L¹ is independently —OP(O)(OH)—, or —OP(S)(OH)—. In certain embodiments, L¹ is —OP(O)(OH)—.

In certain embodiments, X³ is independently: **—C₃-C₁₂ alkylene-L¹-*;

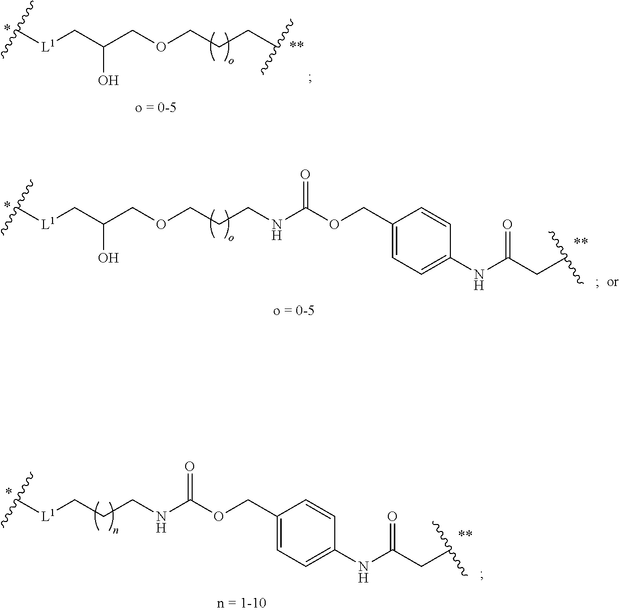

wherein L¹ is independently —OP(O)(OH)—, or —OP(S)(OH)—; * indicates the attachment point connecting to a 3′-O or 5′-O of a terminal nucleotide of the CpG; ** indicates the attachment point connecting to the amino group.

In certain embodiments of the conjugates of formula (X) and (X-1), X¹ is selected from the group consisting of:

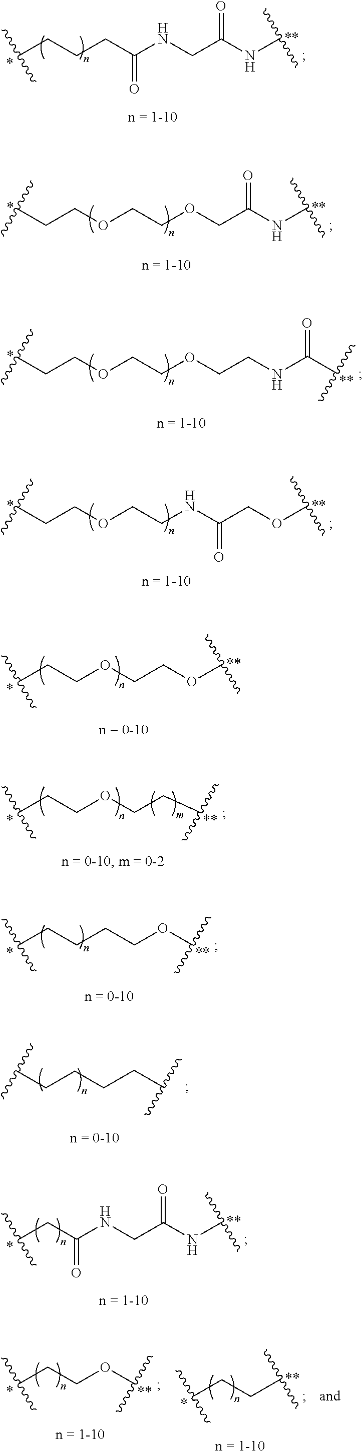

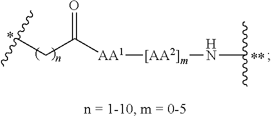

wherein AA¹-[AA²]ₘ comprises the groups selected from: Gly, Lys, Val-Ala, Val-Lys, Val-Cit, Ala-Lys, Phe-Lys, Phe-Cit, Leu-Cit, Ile-Cit, Trp-Cit, Phe-Arg, Phe-Leu, Ala-Phe, Met-Lys, Asn-Lys, Ile-Pro, Ile-Val, Asp-Val, His-Val, Met-(D)Lys, Asn-(D)Lys, Val-(D)Asp, NorVal-(D)Asp, Ala-(D)Asp, Me₃Lys-Pro, PhenylGly-(D)Lys, Pro-(D)Lys, Met-Cit-Val, Gly-Cit-Val, Phe-Phe-Lys, (D)Phe-Phe-Lys, (D)Ala-Phe-Lys, Gly-Phe-Lys, Glu-Val-Cit, Ser-Val-Cit, Gly-Phe-Leu-Gly, and Ala-Leu-Ala-Leu;
wherein * indicates the attachment point connecting to selected T, ** indicates the attachment point connecting to Ar¹.

In certain embodiments of the conjugate of formula (X) and (X-1), X¹ is selected from the group consisting of:

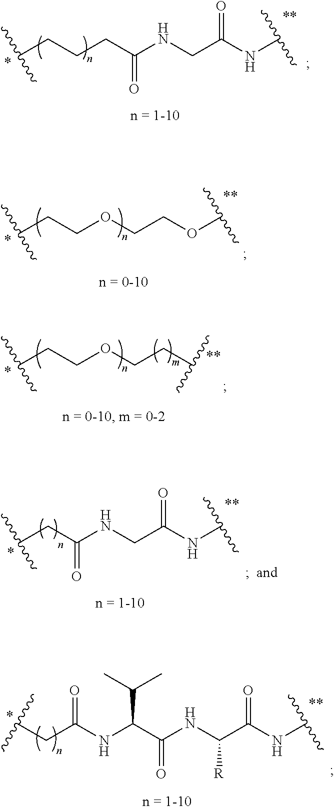

wherein R is methyl or

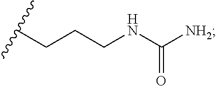 [SMILES: *CCCNC(N)=O]

indicates the attachment point connecting to selected T, ** indicates the attachment point connecting to Ar¹.

In certain embodiments of the conjugate of formula (X) and (X-1), R¹, R², R³ and R⁴ are hydrogen; and/or each Y¹, Y₂, Y³, Y⁴, Y⁵, Y⁶, and Y⁷ is O.

In certain embodiments of the conjugate of formula (X) and (X-1), X³ is

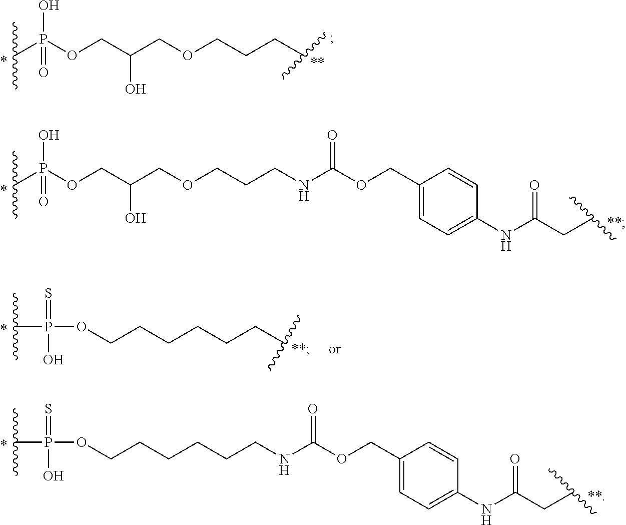 [SMILES: *CCCOCC(O)COP(*)(=O)O ; *CC(=O)Nc1ccc(COC(=O)NCCCOCC(O)COP(*)(=O)O)cc1 ; *CCCCCCOP(*)(O)=S ; *CC(=O)Nc1ccc(COC(=O)NCCCCCCOP(*)(O)=S)cc1]

In certain embodiments, X³ is

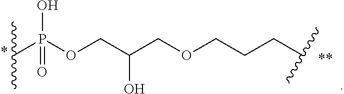 [SMILES: *CCCOCC(O)COP(*)(=O)O]

In certain embodiments, X³ is

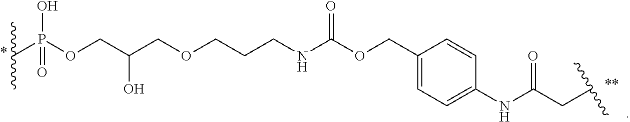 [SMILES: *CC(=O)Nc1ccc(COC(=O)NCCCOCC(O)COP(*)(=O)O)cc1]

In certain embodiments, X³ is

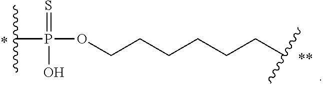 [SMILES: *CCCCCCOP(*)(O)=S]

In certain embodiments, X³ is

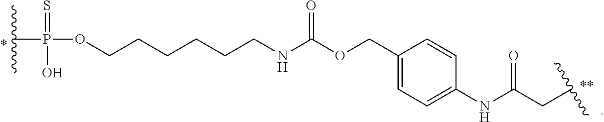 [SMILES: *CC(=O)Nc1ccc(COC(=O)NCCCCCCOP(*)(O)=S)cc1]

In certain embodiments of the conjugate of formula (X) and (X-1):
X¹ is:

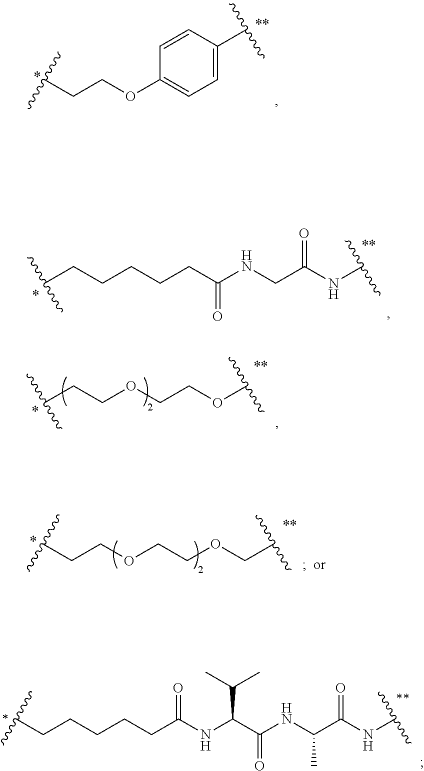

and/or
X², is

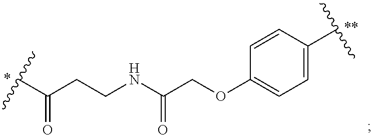 [SMILES: *C(=O)CCNC(=O)COc1ccc(*)cc1]

and/or
X⁴ is

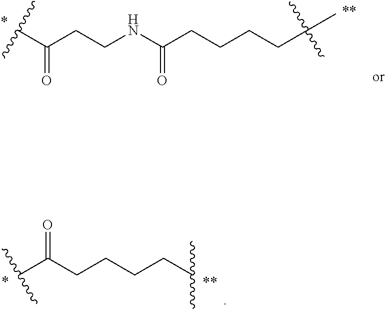 [SMILES: *CCCCC(=O)NCCC(*)=O ; *CCCCC(*)=O]

In certain embodiments, X¹ is:

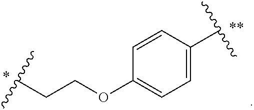 [SMILES: *CCOc1ccc(*)cc1]

In certain embodiments, X¹ is:

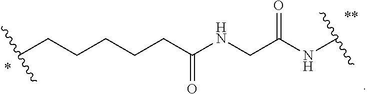 [SMILES: *CCCCCC(=O)NCC(=O)N*]

In certain embodiments, X¹ is:

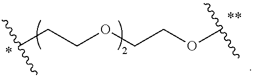

In certain embodiments, X¹ is:

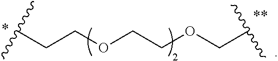

In certain embodiments, X¹ is:

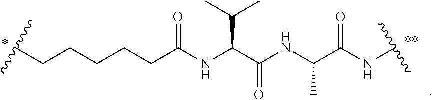 [SMILES: *CCCCCC(=O)N[C@H](C(=O)N[C@@H](C)C(=O)N*)C(C)C]

In certain embodiments, X⁴ is:

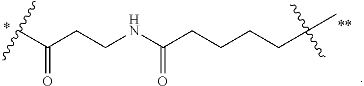 [SMILES: *CCCCC(=O)NCCC(*)=O]

In certain embodiments, X⁴ is

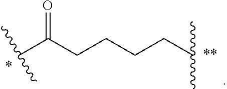 [SMILES: *CCCCC(*)=O]

In certain embodiments, b1 is 1 and b2 is 0, or b1 is 1 and b2 is 1. In certain embodiments, b1 is 1 and b2 is 0. In certain embodiments, b1 is 1 and b2 is 1. In certain embodiments, b2 is 1 and b1 is 0. In certain embodiments when b1 is 0 or b2 is 0, one end of X³ is attached to the 3′-O of the terminal nucleotide of the CpG. In certain embodiments, when b1 is 0 or b2 is 0, one end of X³ is attached to the 5′-O of the terminal nucleotide of the CpG.

In certain embodiments, the triazole functional group is selected from the group consisting of:

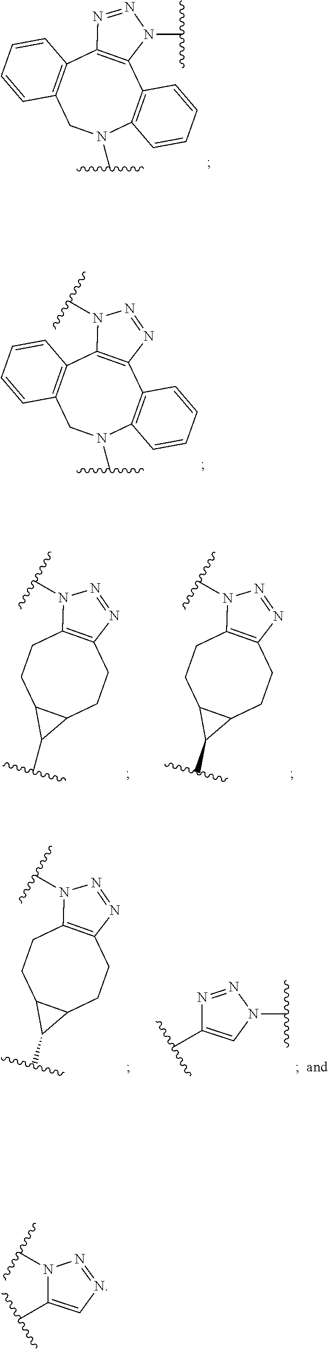

In certain embodiments, the triazole functional group is selected from the group consisting of:

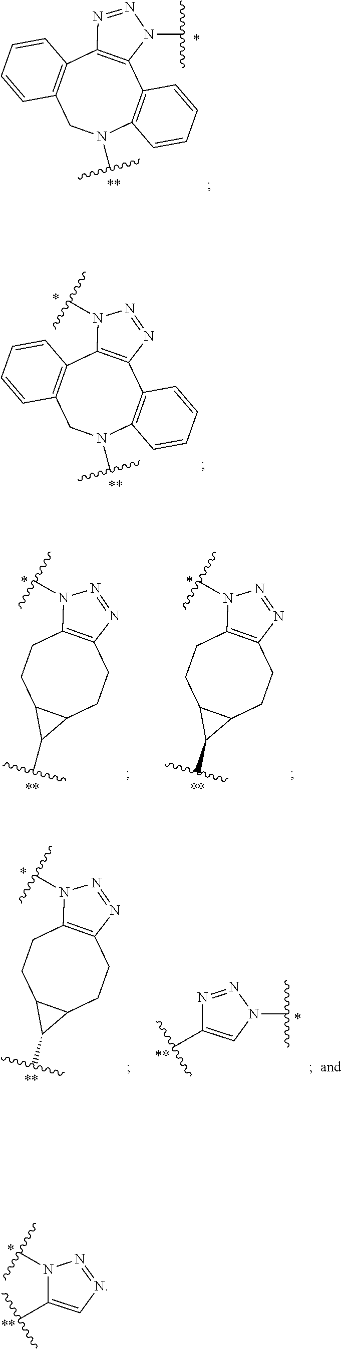

In certain embodiments, T is connected to —Z²-A² through * and T is connected to —Z¹— or —Z³— through **; In certain embodiments, T is connected to —Z²-A² through ** and T is connected to —Z¹— or —Z³— through *.

In certain embodiments, the triazole functional group is

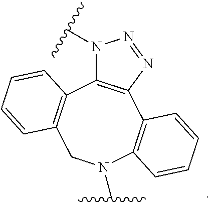 [SMILES: *N1Cc2ccccc2-c2c(nnn2*)-c2ccccc21]

In certain embodiments, the triazole functional group is

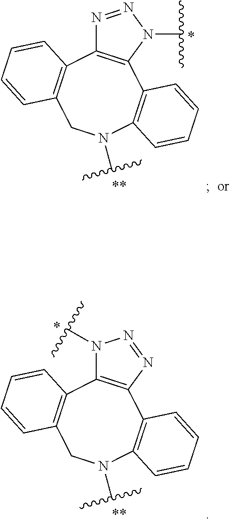 [SMILES: *N1Cc2ccccc2-c2nnn(*)c2-c2ccccc21 ; *N1Cc2ccccc2-c2c(nnn2*)-c2ccccc21]

In certain embodiments, T is connected to —Z²-A² through * and T is connected to —Z¹— or —Z³— through **; In certain embodiments, T is connected to —Z²-A² through ** and T is connected to —Z¹— or —Z³-through *.

In certain embodiments of the conjugate of formula (X), the conjugate has a structure according to formula (XXIV):

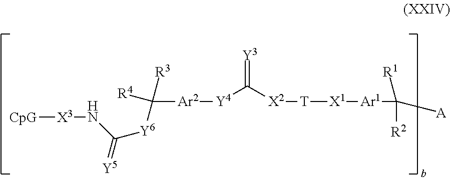

wherein:
Ar¹ and Ar² are each independently a substituted or unsubstituted aryl, or substituted or unsubstituted heteroaryl;
X¹, X² and X³ are each independently a spacer moiety;
R¹, R², R³ and R⁴ are each independently a hydrogen, alkyl, substituted alkyl, substituted or unsubstituted alkenyl, substituted or unsubstituted alkynyl, aryl, or substituted aryl; or
R¹ and R², or R³ and R⁴ together with the atom to which they are attached, can join together to form a 3-8 membered ring that can optionally contain one or two heteroatoms;
Y³, Y⁴, Y⁵ and Y⁶ are each independently O, NH or S;
b is an integer of 1 or 2; wherein when b=2, both groups are directly bound to A;
each T is independently a triazole functional group;
CpG is a TLR9 agonist oligodeoxynucleotide; and
A is a STING agonist.

In certain embodiments, the spacer is covalently bound to a 3′-O of a terminal nucleotide of CpG. In certain embodiments, the spacer is covalently bound to a 5′-O of a terminal nucleotide of CpG.

In certain embodiments of the conjugate of formula (X), the conjugate has a structure according to formula (XXV):

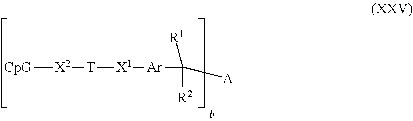

wherein:
Ar is substituted or unsubstituted aryl, or substituted or unsubstituted heteroaryl;
X¹ and X² are each independently a spacer moiety;
R¹ and R² are each independently a hydrogen, alkyl, substituted alkyl, substituted or unsubstituted alkenyl, substituted or unsubstituted alkynyl, aryl, or substituted aryl; or R¹ and R² together with the atom to which they are attached, can join together to form a 3-8 membered ring that can optionally contain one or two heteroatoms;
b is an integer of 1 or 2; wherein when b=2, both groups are directly bound to A;
each T is independently a triazole functional group;
CpG is a TLR9 agonist oligodeoxynucleotide; and
A is a STING agonist.

In certain embodiments of formula (XXV), X² is —X³—NH—CO—X⁴—, and X¹, and X³ and X⁴ are each independently a spacer moiety.

In certain embodiments, the spacer is covalently bound to a 3′-O of a terminal nucleotide of CpG. In certain embodiments, the spacer is covalently bound to a 5′-O of a terminal nucleotide of CpG.

In certain embodiments of the conjugate of formula (X), (X-1), (XXIV) and (XXV), the STING agonist is a STING agonist disclosed in U.S. application Ser. No. 16/643,127, ADU-S100, MK-1454, BMS-986301, GSK3745417, E7766, SB11285,

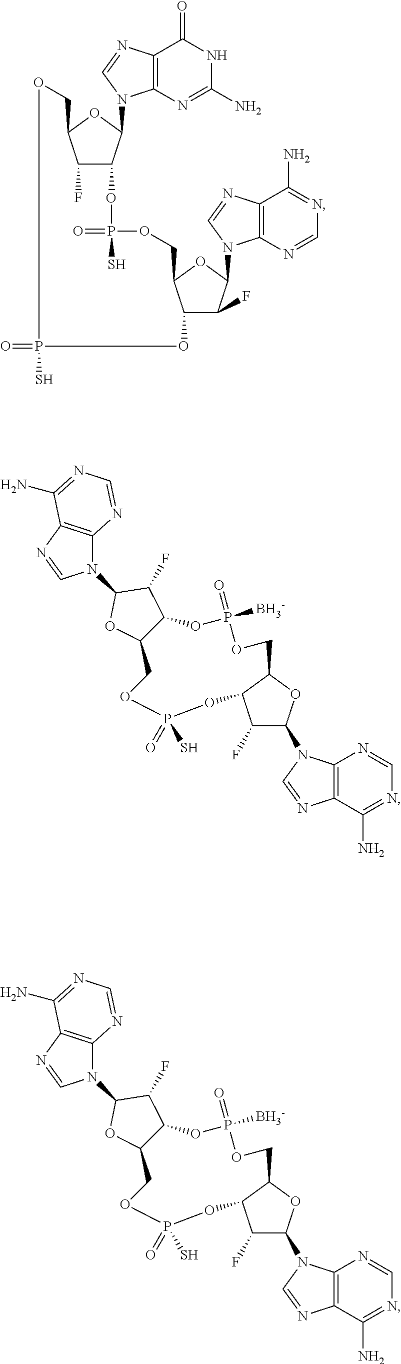

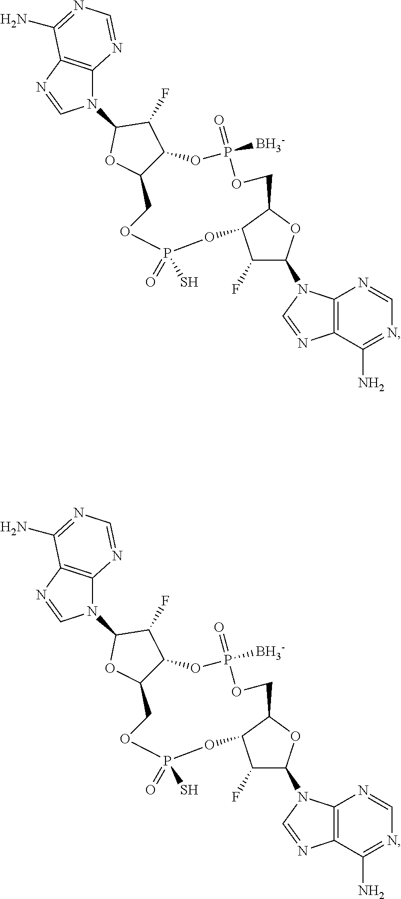

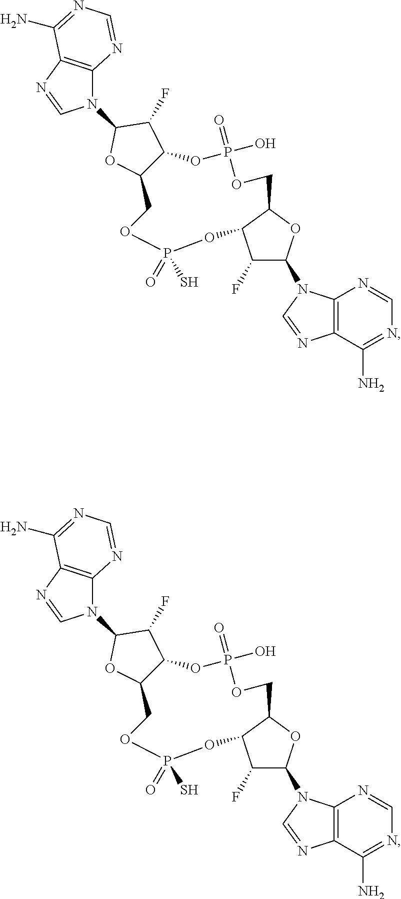 [SMILES: Nc1ncnc2c1ncn2[C@@H]1O[C@@H]2CO[P@@](=O)(S)O[C@H]3[C@@H](F)[C@H](n4cnc5c(N)ncnc54)O[C@@H]3COP(=O)(O)O[C@H]2[C@H]1F ; Nc1ncnc2c1ncn2[C@@H]1O[C@@H]2CO[P@](=O)(S)O[C@H]3[C@@H](F)[C@H](n4cnc5c(N)ncnc54)O[C@@H]3COP(=O)(O)O[C@H]2[C@H]1F]

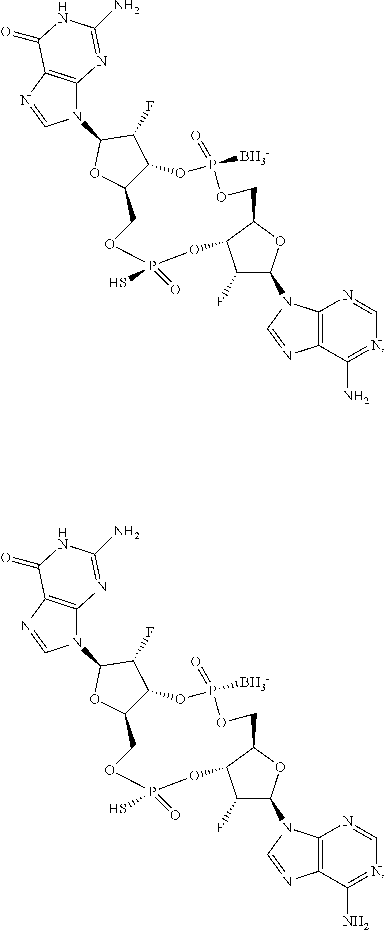

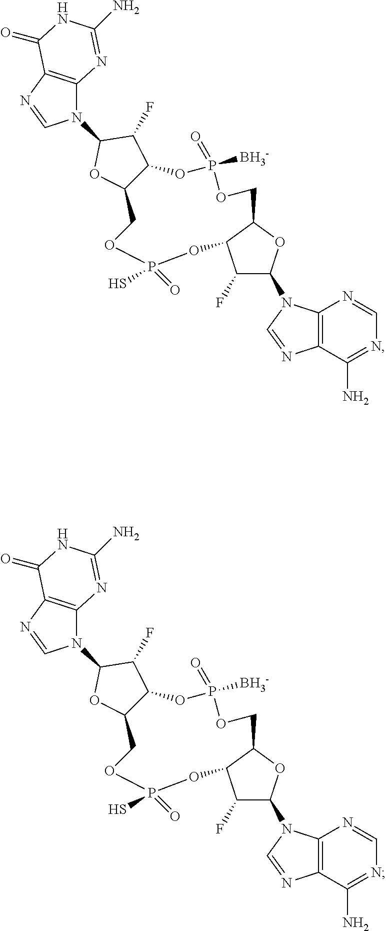

or a stereoisomer thereof.

In certain embodiments, the conjugate of formula (X) has a structure according to formula (XI):

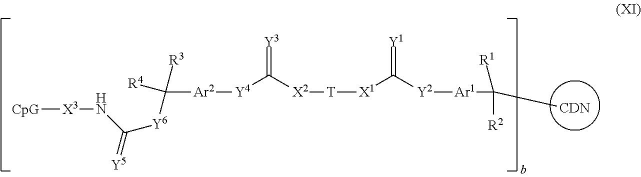

wherein:
Ar¹ and Ar² are each independently a substituted or unsubstituted aryl or heteroaryl;
X¹, X² and X³ are each independently a spacer moiety;
R¹, R², R³ and R⁴ are each independently a hydrogen, alkyl, substituted alkyl, substituted or unsubstituted alkenyl, substituted or unsubstituted alkynyl, aryl, or substituted aryl; or
R¹ and R², or R³ and R⁴ together with the atom to which they are attached, can join together to form a 3-8 membered ring that can optionally contain one or two heteroatoms;
Y¹, Y², Y³, Y⁴, Y⁵ and Y⁶ are each independently O, NH or S;
b is an integer of 1 or 2; wherein when b=2, both groups are directly bound to CDN;
each T is independently a triazole functional group;
CpG is a TLR9 agonist oligodeoxynucleotide; and
CDN is a cyclic dinucleotide that is a STING agonist; and the linker is covalently bound to the cyclic dinucleotide STING agonist.

In certain embodiments, the spacer is covalently bound to 3′-O of the terminal nucleotide of CpG. In certain embodiments, the spacer is covalently bound to 5′-O of the terminal nucleotide of CpG.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XI-A):

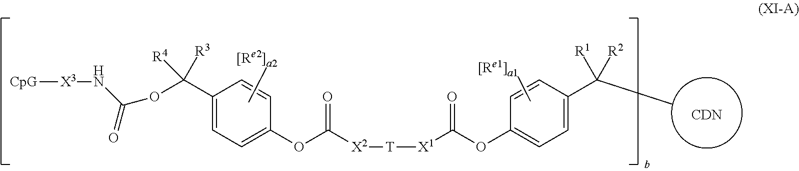

wherein:
X¹ and X² are each independently selected from spacers C1 to C17 as described herein;
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
X³ is selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; ** indicates the attachment point connecting to the amino group;
R¹, R², R³ and R⁴ are each independently a hydrogen, Me, or Et;
each T is independently a triazole functional group;
a1 and a2 are each independently an integer of 0 to 2;
b is an integer of 1 or 2; wherein when b=2, both groups are directly bound to CDN;
R^{e1} and R^{e2} are each independently selected from nitro, cyano, halogen, —OMe, and —OCF₃;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the spacer is covalently bound to a 3′-O of the terminal nucleotide of CpG. In certain embodiments, the spacer is covalently bound to a 5′-O of the terminal nucleotide of CpG.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XI-B):

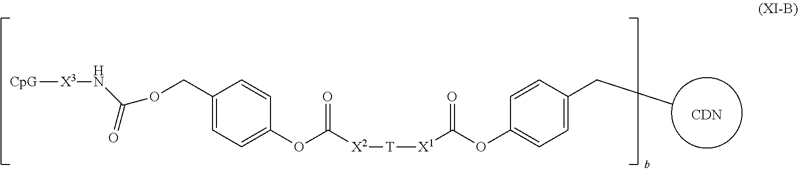

wherein:
X¹ and X² are each independently selected from spacers C1, C3, C8, C10, C13, C18 and C19 as described herein;
   wherein * indicates the attachment point connecting to T, ** indicates the attachment point connecting to the carbonyl group, and the spacer without * or ** indicates each of the two attachment points can be connected to either T or the carbonyl group;
X³ is selected from **—C₃-C₁₂ alkylene-L¹-*; or spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; ** indicates the attachment point connecting to the amino group;
b is an integer of 1 or 2; wherein when b=2, both groups are directly bound to CDN;
each T is independently a triazole functional group;
CpG is a TLR9 agonist oligodeoxynucleotide;
CDN is a cyclic dinucleotide that is a STING agonist; and the linker is covalently bound to the cyclic dinucleotide STING agonist.

In certain embodiments, the spacer is covalently bound to a 3′-O of a terminal nucleotide of CpG. In certain embodiments, the spacer is covalently bound to a 5′-O of a terminal nucleotide of CpG.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments of conjugates of formula (XI-B), wherein X¹ is selected from spacers C8, and C10 as described herein; X² is selected from spacer C19 as described herein: wherein * indicates the attachment point connecting to T, and ** indicates the attachment point connecting to the carbonyl group; X³ is selected from spacer B3 as described herein: wherein L¹ is independently —OP(O)(OH)—, or —OP(S)(OH)—; and * indicates the attachment point connecting to a 3′-O terminal nucleotide of the CpG; and ** indicates the attachment point connecting to the amino group; b is an integer of 1 or 2; wherein when b=2, both groups are directly bound to CDN; each T is independently a triazole functional group; CDN is a cyclic dinucleotide that is a STING agonist; and the linker is covalently bound to a thiol group of the cyclic dinucleotide STING agonist. In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101), connecting at a 3′-O terminal nucleotide of CpG.

In certain embodiments of conjugates of formula (XI-B), X¹ is spacer C10 as described herein.

In certain embodiments, the conjugate of formula (X) has a structure according to formula (XXVI):

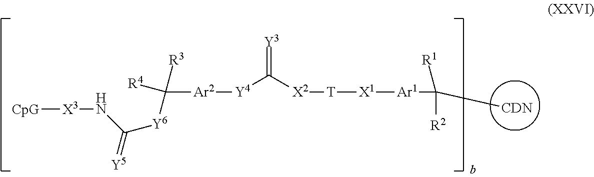

wherein:
Ar¹ and Ar² are each independently a substituted or unsubstituted aryl, or substituted or unsubstituted heteroaryl;
X¹, X² and X³ are each independently a spacer moiety;
R¹, R², R³ and R⁴ are each independently a hydrogen, alkyl, substituted alkyl, substituted or unsubstituted alkenyl, substituted or unsubstituted alkynyl, aryl, or substituted aryl; or
R¹ and R², or R³ and R⁴ together with the atom to which they are attached, can join together to form a 3-8 membered ring that can optionally contain one or two heteroatoms;
Y³, Y⁴, Y⁵ and Y⁶ are each independently O, NH or S;
b is an integer of 1 or 2; wherein when b=2, both groups are directly bound to CDN;
each T is independently a triazole functional group;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the spacer is covalently bound to a 3′-O of a terminal nucleotide of CpG. In certain embodiments, the spacer is covalently bound to a 5′-O of a terminal nucleotide of CpG.

In certain embodiments, the conjugate of formula (X) has a structure according to formula (XXVI-A):

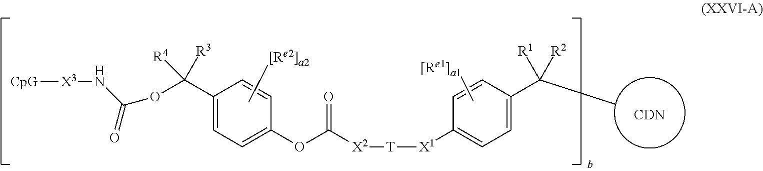

wherein:
   X¹ is selected from spacers D1 to D12 as described herein; wherein * indicates the attachment point connecting to selected T, ** indicates the attachment point connecting to the phenyl group, and the spacer without * or ** indicates the attachment points can be connected to either T or the phenyl group;
X² is selected from spacers C13 to C17 as described herein: wherein oxygen can be optionally replaced by NH, NMe, NAc, S, or SO₂; * indicates the attachment point connecting to T, and ** indicates the attachment point connecting to the carbonyl group;
X³ is selected from: **—C₃-C₁₂ alkylene-L¹-*, and spacers B1 to B5 as described herein: wherein L¹ is independently a bond, —OP(O)(OH)—, or —OP(S)(OH)—, * indicates the attachment point connecting to a 3′-O or 5′-O of a terminal nucleotide of the CpG; and ** indicates the attachment point connecting to the amino group;
R¹, R², R³ and R⁴ are each independently a hydrogen, Me, or Et;
each T is independently a triazole functional group;
a1 and a2 are each independently an integer of 0 to 2;
b is an integer of 1 or 2; wherein when b=2, both groups are directly bound to CDN;
R^{e1} and R^{e2} are each independently selected from nitro, cyano, halogen, —OMe, and —OCF₃;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XXVI-B):

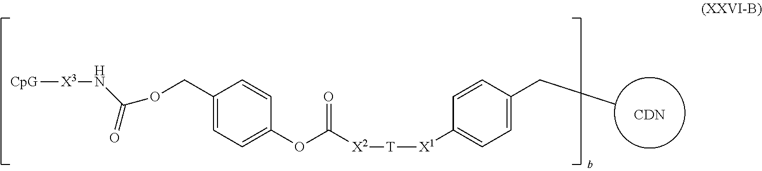

wherein:
X¹ is selected from spacers D1, D5 D6, D9 and D13 as described herein: wherein * indicates the attachment point connecting to selected T, and ** indicates the attachment point connecting to the phenyl group;
X² is selected from spacer C19 as described herein; wherein * indicates the attachment point connecting to T, and ** indicates the attachment point connecting to the carbonyl group;
X³ is selected from spacer B3 as described herein: wherein L¹ is independently —OP(O)(OH)—, or —OP(S)(OH)—, * indicates the attachment point connecting to a 3′-O of a terminal nucleotide of the CpG; ** indicates the attachment point connecting to the amino group;
b is an integer of 1 or 2; wherein when b=2, both groups are directly bound to CDN;
each T is independently a triazole functional group;
CDN is a cyclic dinucleotide that is a STING agonist; and
the linker is covalently bound to a thiol group of the cyclic dinucleotide STING agonist.

In certain embodiments of conjugates of formula (XXVI-B), X¹ is selected from spacers D9 and D13 as described herein. In certain embodiments of conjugates of formula (XXVI-B), R is methyl in spacer D13.

In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101), connecting at 3′-O of a terminal nucleotide.

In certain embodiments, the conjugate of formula (X) has a structure according to formula (XII):

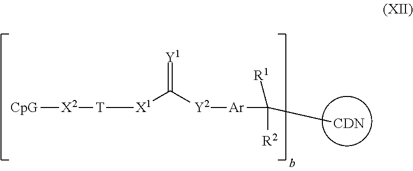

wherein:
Ar is a substituted or unsubstituted aryl, or substituted or unsubstituted heteroaryl;
X¹ and X² are each independently a spacer moiety;
R¹ and R² are each independently a hydrogen, alkyl, substituted alkyl, substituted or unsubstituted alkenyl, substituted or unsubstituted alkynyl, aryl, or substituted aryl; or R¹ and R² together with the atom to which they are attached, can join together to form a 3-8 membered ring that can optionally contain one or two heteroatoms;
Y¹ and Y² are each independently O, NH or S;
b is an integer of 1 or 2; wherein when b=2, both groups are directly bound to CDN;
each T is independently a triazole functional group;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the spacer is covalently bound a 3′-O of a terminal nucleotide of CpG. In certain embodiments, the spacer is covalently bound to a 5′-O of a terminal nucleotide of CpG.

In certain embodiments, the conjugate of formula (X) has a structure according to formula (XII-A):

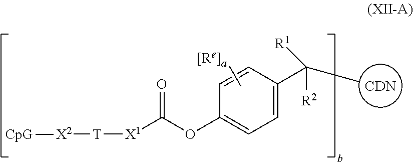

wherein:
X¹ is selected from spacers C1 to C17 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
X² is —X³—NH—CO—X⁴—, wherein X³ is selected from **—C₃-C₁₂ alkylene-L¹-*; and spacers B1 to B6 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
X⁴ is selected from spacers C1, C3, C8, C10, C13, C18 and C19 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
R¹ and R² are each independently hydrogen, Me, or Et;
each T is independently a triazole functional group;
a is an integer of 0 to 2;
b is an integer of 1 or 2; wherein when b=2, both groups are directly bound to CDN;
each R^{e1} and R^{e2} is independently selected from nitro, cyano, halogen, —OMe, —NHMe, —NHAc, —NHSO₂Me, and —OCF₃;
CpG is a TLR9 agonist oligodeoxynucleotide;
CDN is a cyclic dinucleotide that is a STING agonist; and the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the spacer is covalently bound to a 3′-O of a terminal nucleotide of CpG. In certain embodiments, the spacer is covalently bound to a 5′-O of a terminal nucleotide of CpG.

In certain embodiments, the conjugate of formula (X) has a structure according to formula (XII-B):

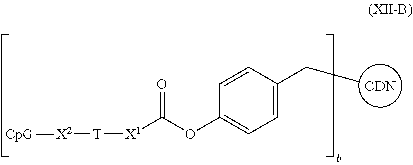

wherein:
X¹ is selected from spacers C1, C3, C8, C10, C13, C18 and C19 as described herein:
   wherein * indicates the attachment point connecting to T, ** indicates the attachment point connecting to the carbonyl group, and the spacer without * or ** indicates each of the two attachment points can be connected to either T or the carbonyl group;
X² is —X³—NH—CO—X⁴—, wherein X³ is selected from **—C₃-C₁₂ alkylene-L¹-*; and spacers B1 to B6 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
X⁴ is selected from spacers C1, C3, C8, C10, C13, C18 and C19 as described herein:
   wherein * indicates the attachment point connecting to T, ** indicates the attachment point connecting to the carbonyl group, and the spacer without * or ** indicates each of the two attachment points can be connected to either T or the carbonyl group;
each T is independently a triazole functional group;
b is an integer of 1 or 2; wherein when b=2, both groups are directly bound to CDN;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the spacer is covalently bound to a 3′-O of a terminal nucleotide of CpG. In certain embodiments, the spacer is covalently bound to a 5′-O of a terminal nucleotide of CpG.

In certain embodiments of conjugates of formula (XII-B), wherein: X¹ is selected from spacers C8 and C10 as described herein: wherein * indicates the attachment point connecting to T, and ** indicates the attachment point connecting to the carbonyl group; X² is —X³—NH—CO—X⁴—, wherein X³ is selected from spacers B3 and B6 as described herein: wherein L¹ is independently —OP(O)(OH)—, or —OP(S)(OH)—; and * indicates the attachment point connecting to a 3′-O of a terminal nucleotide of the CpG; and ** indicates the attachment point connecting to the amino group; or X³ is: **—C₃-C₁₂ alkylene-L¹-*; wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; X⁴ is selected from spacers C13 and C18 as described herein: wherein * indicates the attachment point connecting to T, and ** indicates the attachment point connecting to the carbonyl group; each T is independently a triazole functional group; b is an integer of 1 or 2; wherein when b=2, both groups are directly bound to CDN; CDN is a cyclic dinucleotide that is a STING agonist; and the linker is covalently bound to a thiol group of the cyclic dinucleotide STING agonist. In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101); connecting at a 3′-O or 5′-O of a terminal nucleotide of the CpG.

In certain embodiments of conjugates of formula (XII-B), wherein: X¹ is spacer C10 as described herein.

In certain embodiments, the conjugate of formula (X) has a structure according to formula (XXVII):

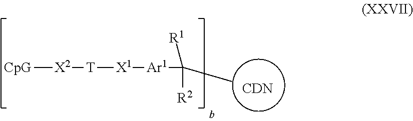

wherein:
Ar is substituted or unsubstituted aryl, or substituted or unsubstituted heteroaryl;
X¹ and X² are each independently a spacer moiety;
R¹ and R² are each independently a hydrogen, alkyl, substituted alkyl, substituted or unsubstituted alkenyl, substituted or unsubstituted alkynyl, aryl, or substituted aryl; or R¹ and R² together with the atom to which they are attached, can join together to form a 3-8 membered ring that can optionally contain one or two heteroatoms;
b is an integer of 1 or 2; wherein when b=2, both groups are directly bound to CDN;
each T is independently a triazole functional group;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XXVII-A):

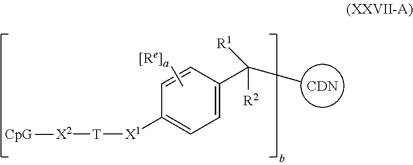

wherein:
X¹ is selected from spacer D1 to D12 as described herein: wherein * indicates the attachment point connecting to selected T, ** indicates the attachment point connecting to the phenyl group, and the spacer without * or ** indicates the attachment points can be connected to either T or the phenyl group;
X² is —X³—NH—CO—X⁴—, wherein X³ is selected from: **—C₃-C₁₂ alkylene-L¹-*; and spacer B1 to B7 as described herein: wherein L¹ is independently a bond, —OP(O)(OH)—, or —OP(S)(OH)—, * indicates the attachment point connecting to a 3′-O or 5′-O of a terminal nucleotide of the CpG; and ** indicates the attachment point connecting to the amino group;
X⁴ is selected from spacer C13, C18 and C19 as described herein: wherein oxygen can be optionally replaced by NH, NMe, NAc, S, or SO₂; * indicates the attachment point connecting to T, and ** indicates the attachment point connecting to the carbonyl group;
R¹ and R² are each independently hydrogen, Me, or Et;
each T is independently a triazole functional group;
a is an integer of 0 to 2;
b is an integer of 1 or 2; wherein when b=2, both groups are directly bound to CDN;
R^{e1} and R^{e2} are each independently selected from nitro, cyano, halogen, —OMe, —NHMe, —NHAc, —NHSO₂Me, and —OCF₃;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XXVII-B):

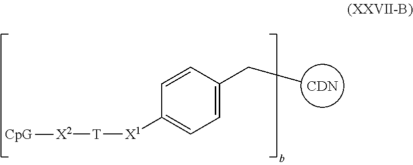

wherein:
X¹ is selected from spacer D1, D5, D6, D9 and D13 as described herein; wherein * indicates the attachment point connecting to T, ** indicates the attachment point connecting to the phenyl group;
X² is —X³—NH—CO—X⁴—, wherein X³ is selected from spacer B3 and B6 as described herein: wherein L¹ is independently —OP(O)(OH)—, or —OP(S)(OH)—, * indicates the attachment point connecting to a 3′-O of a terminal nucleotide of the CpG; and ** indicates the attachment point connecting to the amino group; or X³ is: **—C₃-C₁₂ alkylene-L¹-* or B7 as described herein: wherein L¹ is independently —OP(O)(OH)—, or —OP(S)(OH)—; * indicates the attachment point connecting to a 5′-O of a terminal nucleotide of the CpG; and ** indicates the attachment point connecting to the amino group;
X⁴ is selected from spacer C13 and C18 as described herein: wherein * indicates the attachment point connecting to T, and ** indicates the attachment point connecting to the carbonyl group;
each T is independently a triazole functional group;
b is an integer of 1 or 2; wherein when b=2, both groups are directly bound to CDN;
CDN is a cyclic dinucleotide that is a STING agonist; and
the linker is covalently bound to a thiol group of the cyclic dinucleotide STING agonist.

In certain embodiments of conjugates of formula (XXVII-B), X¹ is selected from spacers D9 and D13 as described herein. In certain embodiments of conjugates of formula (XXVI-B), wherein R is methyl in spacer D13.

In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101), connecting at a 3′-O or 5′-O of a terminal nucleotide of the CpG.

In certain embodiments, the conjugate of formula (X) has a structure according to formula (XIII):

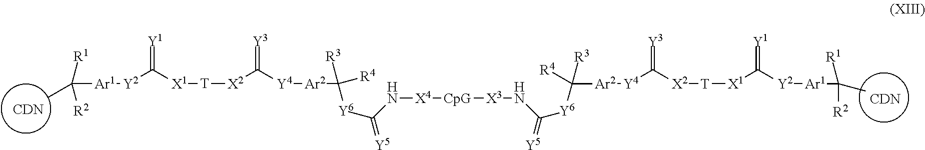

wherein:
Ar¹ and Ar² are each independently a substituted or unsubstituted aryl or heteroaryl;
X¹, X², X³ and X⁴ are each independently a spacer moiety;
R¹, R², R³ and R⁴ are each independently a hydrogen, alkyl, substituted alkyl, substituted or unsubstituted alkenyl, substituted or unsubstituted alkynyl, aryl, or substituted aryl; or
R¹ and R² together with the atom to which they are attached, can join together to form a 3-8 membered ring that can optionally contain one or two heteroatoms; or
R³ and R⁴ together with the atom to which they are attached, can join together to form a 3-8 membered ring that can optionally contain one or two heteroatoms;
Y¹, Y², Y³, Y⁴, Y⁵ and Y⁶ are each independently O, NH or S;
each T is independently a triazole functional group;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XIII-A):

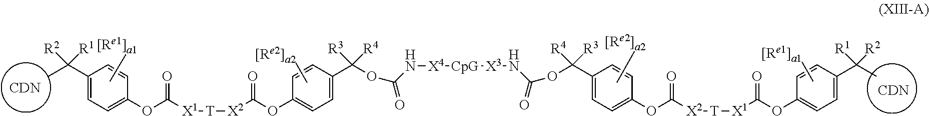

wherein:
X¹ and X² are each independently selected from spacers C1 to C17 as described herein;
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
X³ and X⁴ are each independently selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
R¹, R², R³ and R⁴ are each independently a hydrogen, Me, or Et;
each T is independently a triazole functional group;
a1 and a2 are each independently an integer of 0 to 2;
each R^{e1} and R^{e2} is independently selected from nitro, cyano, halogen, —OMe, and —OCF₃;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XIII-B):

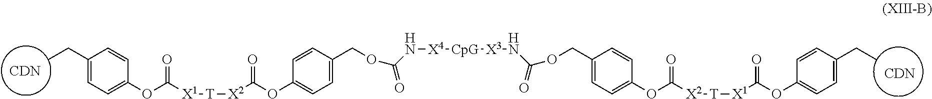

wherein:
X¹ and X² are each independently selected from spacers C1, C3, C8, C10, C13, C18 and C19 as described herein;
wherein * indicates the attachment point connecting to T, ** indicates the attachment point connecting to the carbonyl group, and the spacer without * or ** indicates each of the two attachment points can be connected to either T or the carbonyl group;
X³ and X⁴ are each independently selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
each T is independently a triazole functional group;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments of the conjugate of formula (XIII-B), wherein X¹ is selected from spacers C8 and C10 as described herein and X² is selected from spacer C19 as described herein; wherein * indicates the attachment point connecting to T, and ** indicates the attachment point connecting to the carbonyl group; X⁴ is selected from **—C₃-C₁₂ alkylene-L′-*; wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; and X³ is selected from spacer B3 as described herein: wherein L¹ is independently —OP(O)(OH)—, or —OP(S)(OH)—; and * indicates the attachment point connecting to a 3′-O of a terminal nucleotide of the CpG; ** indicates the attachment point connecting to the amino group; each T is independently a triazole functional group; CDN is a cyclic dinucleotide that is a STING agonist; and the linker is covalently bound to a thiol group of the cyclic dinucleotide STING agonist. In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101), wherein X⁴ is covalently bound to a 5′-O of a terminal nucleotide of CpG and X³ is covalently bound to a 3′-O of a terminal nucleotide of CpG.

In certain embodiments, the conjugate of formula (X) has a structure according to formula (XIV):

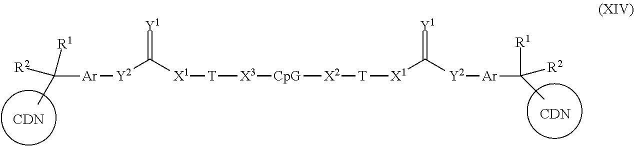

wherein:
Ar is a substituted or unsubstituted aryl, or substituted or unsubstituted heteroaryl;
X¹, X² and X³ are each independently a spacer moiety;
R¹ and R² are each independently a hydrogen, alkyl, substituted alkyl, substituted or unsubstituted alkenyl, substituted or unsubstituted alkynyl, aryl, or substituted aryl; or
R¹ and R² together with the atom to which they attach can join together to form a 3-8 membered ring that can optionally contain one or two heteroatoms;
Y¹ and Y² are each independently O, NH or S;
each T is independently a triazole functional group;
CpG is a TLR9 agonist oligodeoxynucleotide;
CDN is a cyclic dinucleotide that is a STING agonist; and
the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XIV-A):

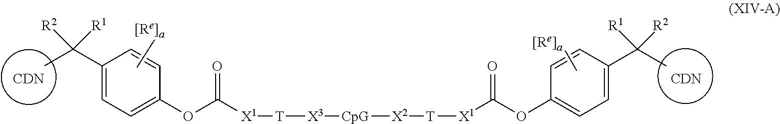

wherein:
X¹ is selected from spacers C1 to C17 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
X² and X³ are each independently —X⁴—NH—CO—X⁵—, wherein X⁴ is selected from **—C₃-C₁₂ alkylene-L¹-*; and spacers B1 to B5 as described herein: wherein L¹ is independently a bond, —OP(O)(OH)—, or —OP(S)(OH)—; and * indicates the attachment point connecting to a 3′-O or 5′-O of a terminal nucleotide of the CpG; ** indicates the attachment point connecting to the amino group;
X⁵ is selected from spacers C1, C3, C8, C10, C13, C18 and C19 as described herein: wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
R¹ and R² are each independently a hydrogen, Me, or Et;
each T is independently a triazole functional group;
a is an integer of 0 to 2;
each R^{e} is independently selected from nitro, cyano, halogen, —OMe, —NHMe, —NHAc, —NHSO₂Me, and —OCF₃;
CpG is a TLR9 agonist oligodeoxynucleotide;
CDN is a cyclic dinucleotide that is a STING agonist; and the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XIV-B):

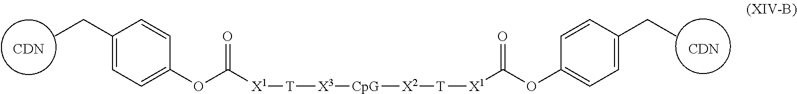

wherein:
X¹ is selected from spacers C1, C3, C8, C10, C13, C18 and C19 as described herein:
   wherein * indicates the attachment point connecting to T, ** indicates the attachment point connecting to the carbonyl group, and the spacer without * or ** indicates each of the two attachment points can be connected to either T or the carbonyl group;
X² and X³ are each independently —X⁴—NH—CO—X⁵—, wherein X⁴ is selected **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; ** indicates the attachment point connecting to the amino group;
X⁵ is selected from spacers C1, C3, C8, C10, C13, C18 and C19 as described herein:
   wherein * indicates the attachment point connecting to T, ** indicates the attachment point connecting to the carbonyl group, and the spacer without * or ** indicates each of the two attachment points can be connected to either T or the carbonyl group;
each T is independently a triazole functional group;
CpG is a TLR9 agonist oligodeoxynucleotide;
CDN is a cyclic dinucleotide that is a STING agonist; and the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments of the conjugate of formula (XIV-B), wherein X¹ is selected from spacers C8 and C10 as described herein: wherein * indicates the attachment point connecting to T and ** indicates the attachment point connecting to the carbonyl group; X² and X³ are each independently —X⁴—NH—CO—X⁵—, wherein X⁴ is **—C₃-C₁₂ alkylene-L¹-*; wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; or X⁴ is spacer B3 as described herein: wherein L¹ is independently —OP(O)(OH)—, or —OP(S)(OH)—; and * indicates the attachment point connecting to a 3′-O of a terminal nucleotide of the CpG; ** indicates the attachment point connecting to the amino group; X⁵ is spacer C13 as described herein: wherein * indicates the attachment point connecting to T, and ** indicates the attachment point connecting to the carbonyl group; each T is independently a triazole functional group; CDN is a cyclic dinucleotide that is a STING agonist; and the linker is covalently bound to a thiol group of the cyclic dinucleotide STING agonist. In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101).

In certain embodiments, the conjugate of formula (X) has a structure according to formula (XV):

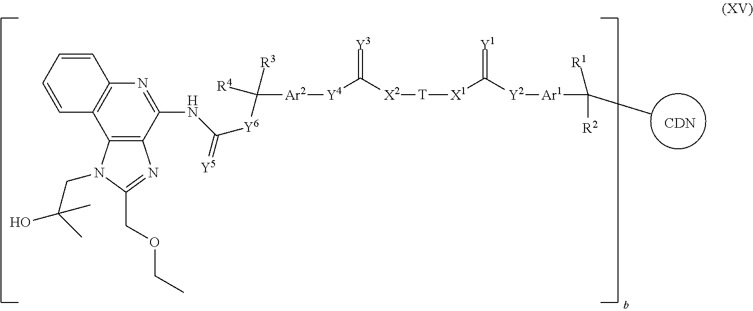

wherein:
Ar¹ and Ar² are each independently a substituted or unsubstituted aryl or heteroaryl;
X¹ and X² are each independently a spacer moiety;
R¹, R², R³ and R⁴ are each independently a hydrogen, alkyl, substituted alkyl, substituted or unsubstituted alkenyl, substituted or unsubstituted alkynyl, aryl, or substituted aryl; or
R¹ and R² together with the atom to which they are attached, can join to form a 3-8 membered ring that can contain one or two heteroatoms; or
R³ and R⁴ together with the atom to which they are attached, can join to form a 3-8 membered ring that can contain one or two heteroatoms;
b is an integer of 1 or 2; wherein when b=2, both groups are directly bound to CDN;
Y¹, Y², Y³, Y⁴, Y⁵ and Y⁶ are each independently O or S;
each T is independently a triazole functional group;
CDN is a cyclic dinucleotide that is a STING agonist; and the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XV-A):

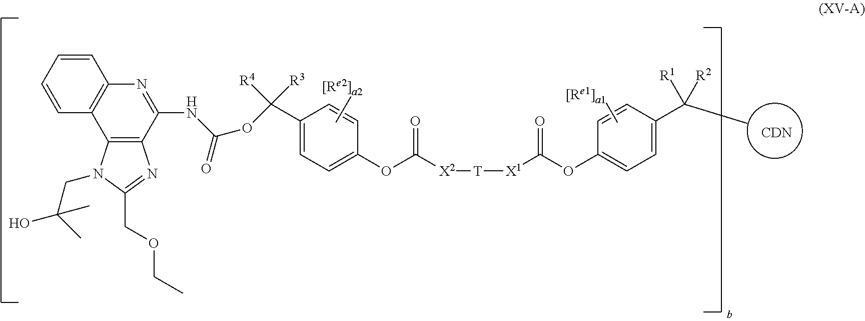

wherein:
X¹ and X² are each independently selected from spacers C1 to C17 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group.
R¹, R², R³ and R⁴ are each independently a hydrogen, Me, or Et;
each T is independently a triazole functional group;
a1 and a2 are each independently an integer of 0 to 2;
b is an integer of 1 or 2; wherein when b=2, both groups are directly bound to CDN;
each R^{e1} and R^{e2} is independently selected from nitro, cyano, halogen, —OMe, and —OCF₃;
CDN is a cyclic dinucleotide that is a STING agonist; and the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments, the conjugate of formula (X) has a structure according to formula (XV-B):

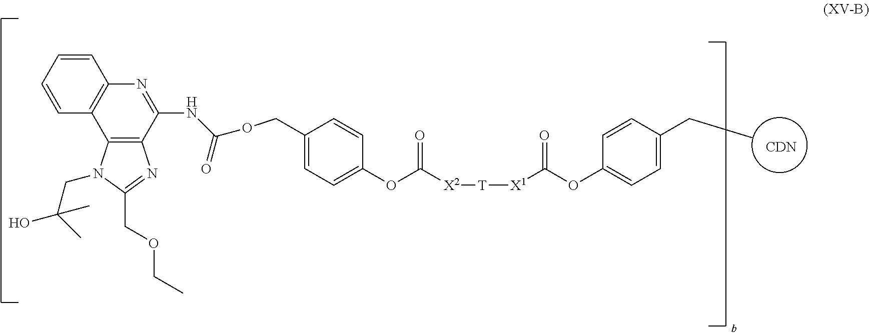

wherein:
X¹ and X² are each independently selected from spacers C1, C3, C8, C10, C13, C18 and C19 as described herein;
   wherein * indicates the attachment point connecting to T, ** indicates the attachment point connecting to the carbonyl group, and the spacer without * or ** indicates each of the two attachment points can be connected to either T or the carbonyl group;
each T is independently a triazole functional group;
b is an integer of 1 or 2; wherein when b=2, both groups are directly bound to CDN;
CDN is a cyclic dinucleotide that is a STING agonist; and the linker is covalently bound to the cyclic dinucleotide STING agonist.

In certain embodiments, the linker is covalently bound to a thiol group of the cyclic dinucleotide STING agonist. In certain embodiments, the linker is covalently bound to a nitrogen of the cyclic dinucleotide STING agonist.

In certain embodiments of the conjugate of formula (XV-B), X¹ is selected from spacers C8 and C10 as described herein; and X² is selected from spacer C13 as described herein: wherein * indicates the attachment point connecting to T, and ** indicates the attachment point connecting to the carbonyl group; each T is independently a triazole functional group; b is an integer of 1 or 2; wherein when b=2, both groups are directly bound to CDN; CDN is a cyclic dinucleotide that is a STING agonist; and the linker is covalently bound to a thiol group of the cyclic dinucleotide STING agonist. In certain embodiments of the conjugate of formula (XV-B), X¹ is C10.

In certain embodiments of the therapeutic agent of formulae (III), (III-A), (III-B), (III-C), (IV), (IV-A), (IV-B), (X), (X-1), (X-A), (X-B), (X-C), (X-D), (X-E), (X-F), (X-G), (X-H), (XI), (XI-A), (XI-B), (XII), (XII-A), (XII-B), (XIII), (XIII-A), (XIII-B), (XIV), (XIV-A), (XIV-B), (XV), (XV-A), (XV-B), (XXII), (XXIII), (XXIV), (XXV), (XXVI), (XXVI-A), (XXVI-B), (XXVII), (XXVII-A) and (XXVII-B), the cyclic dinucleotide is

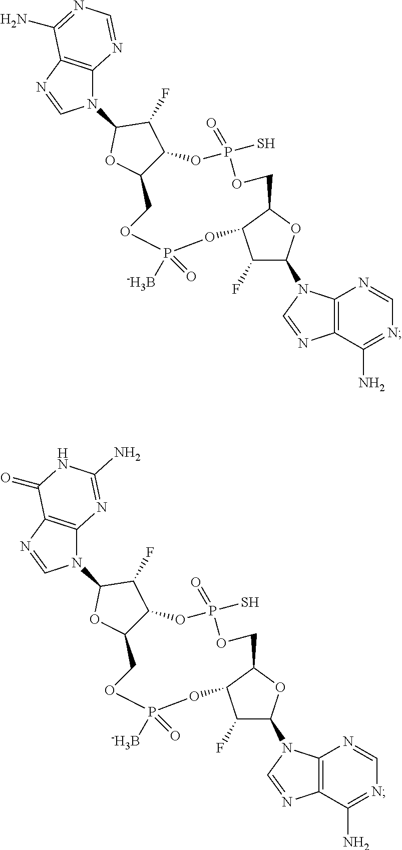

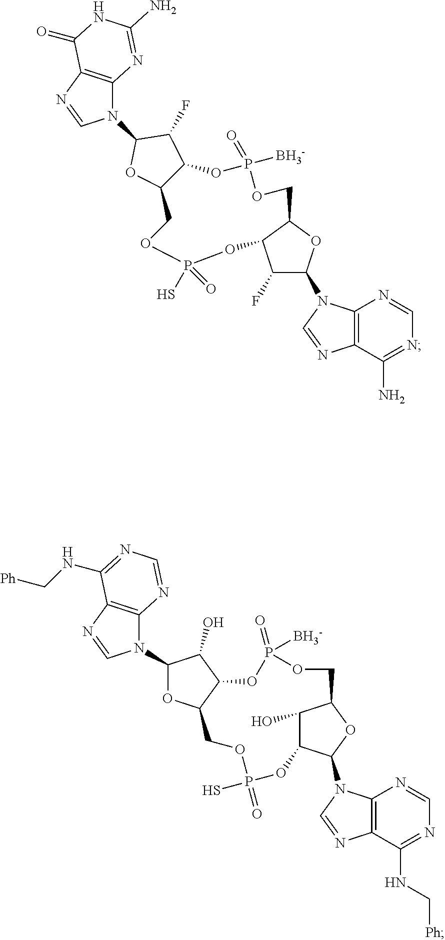

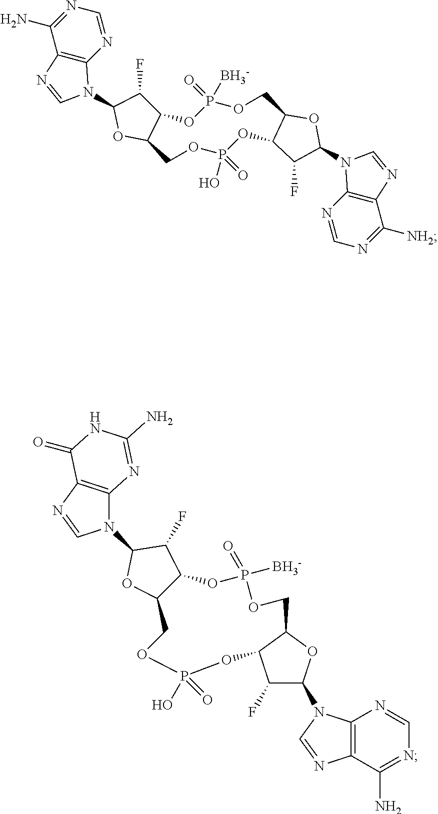

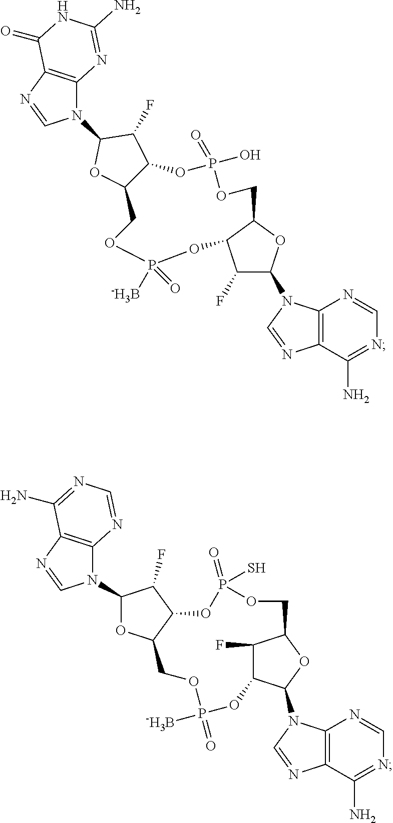

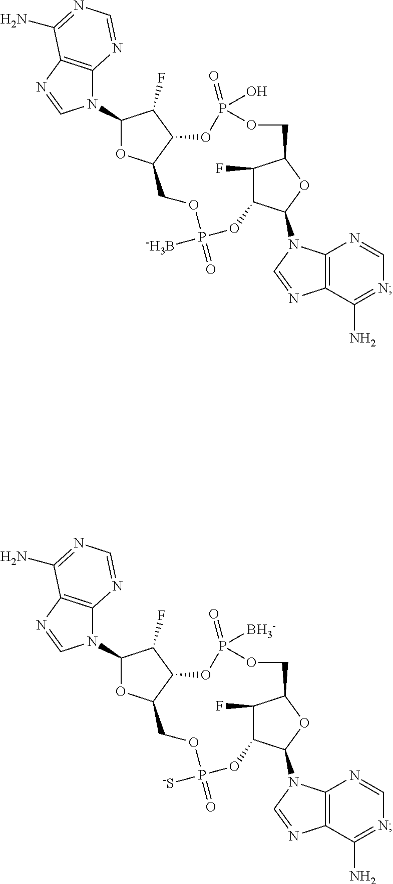

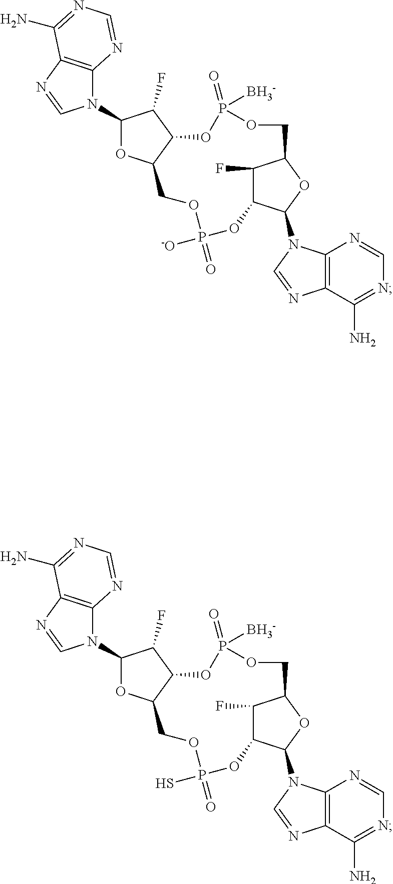

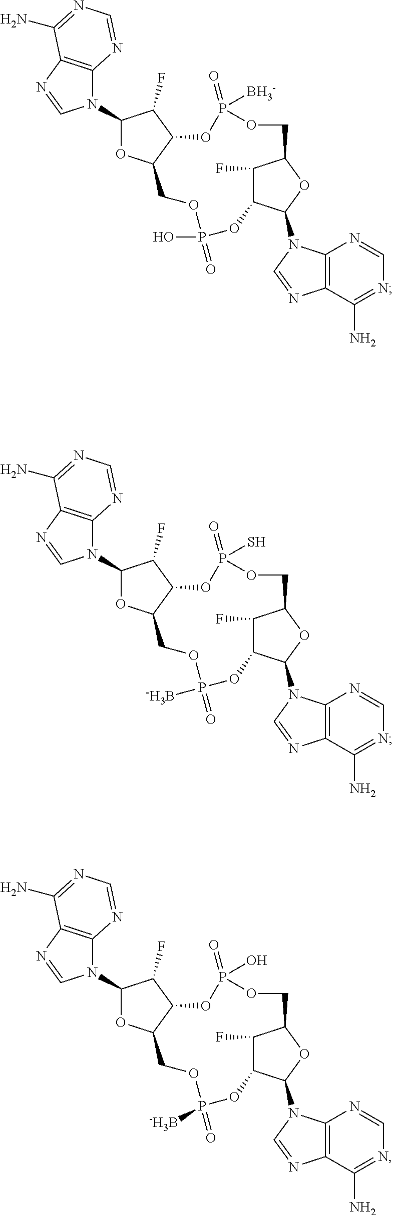

or a pharmaceutically acceptable salt, stereoisomer, solvate or hydrate thereof.

In certain embodiments of the therapeutic agent of formulae (III), (III-A), (III-B), (II-C), (IV), (IV-A), (IV-B), (X), (X-1), (X-A), (X-B), (X-C), (X-D), (X-E), (X-F), (X-G), (X-H), (XI), (XI-A), (XI-B), (XII), (XII-A), (XII-B), (XIII), (XIII-A), (XIII-B), (XIV), (XIV-A), (XIV-B), (XV), (XV-A), (XV-B), (XXII), (XXIII), (XXIV), (XXV), (XXVI), (XXVI-A), (XXVI-B), (XXVII), (XXVII-A) and (XXVII-B), the cyclic dinucleotide is:

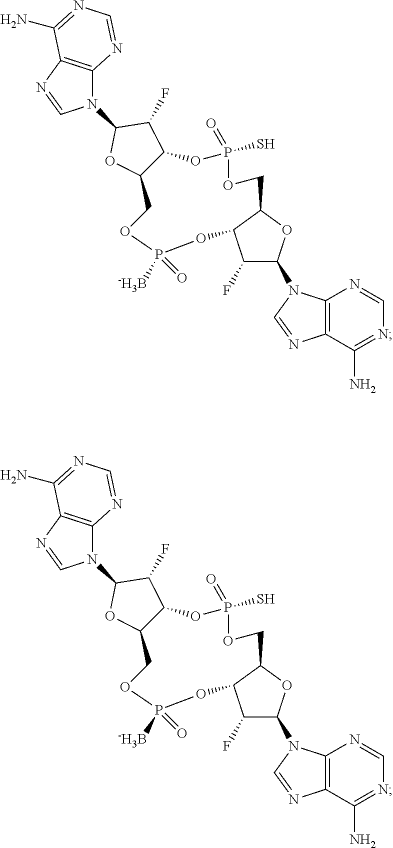

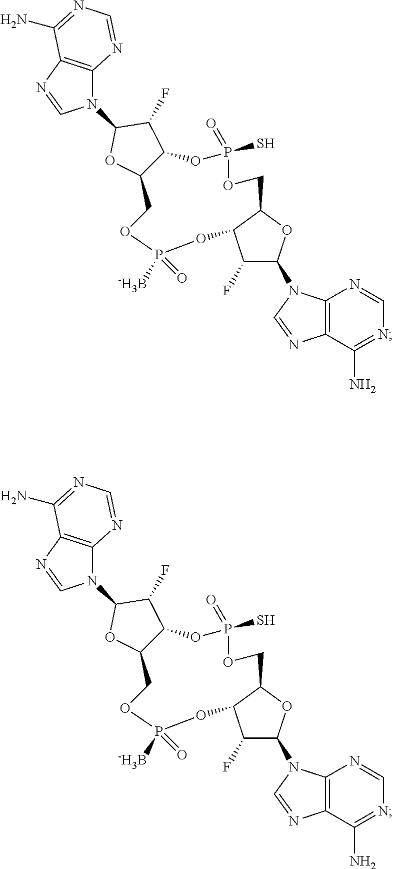

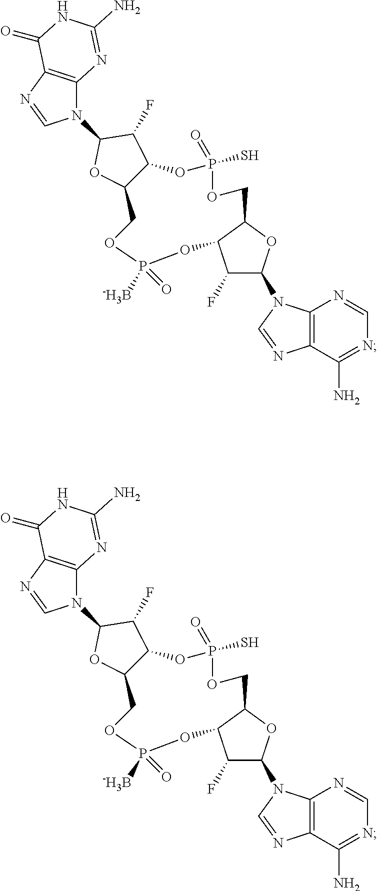

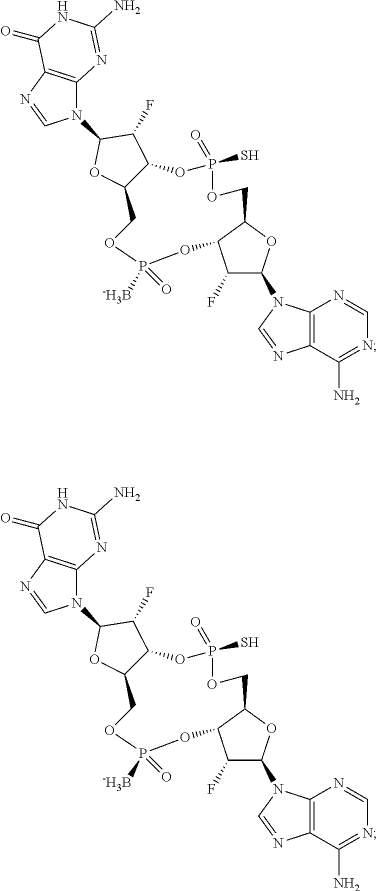

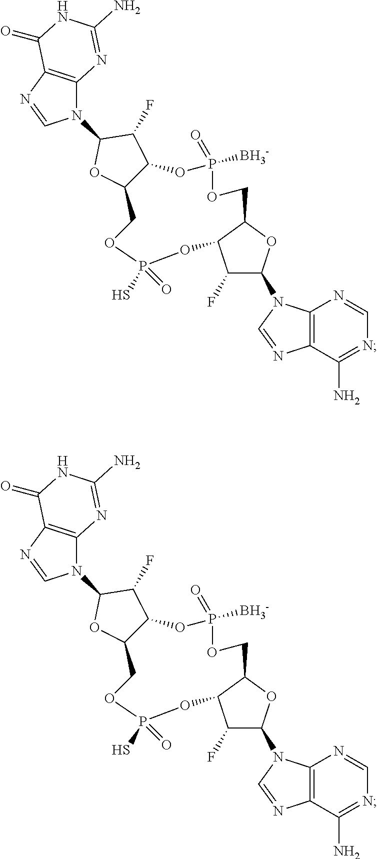

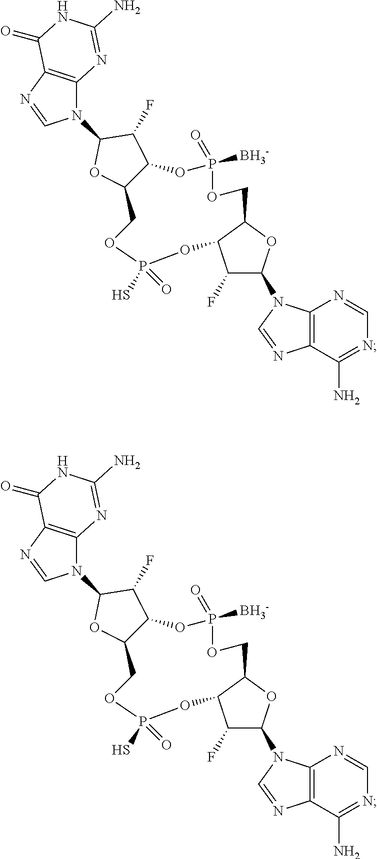

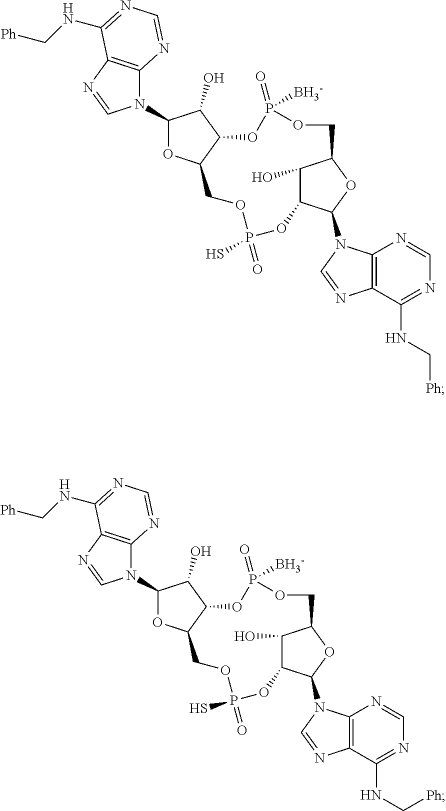

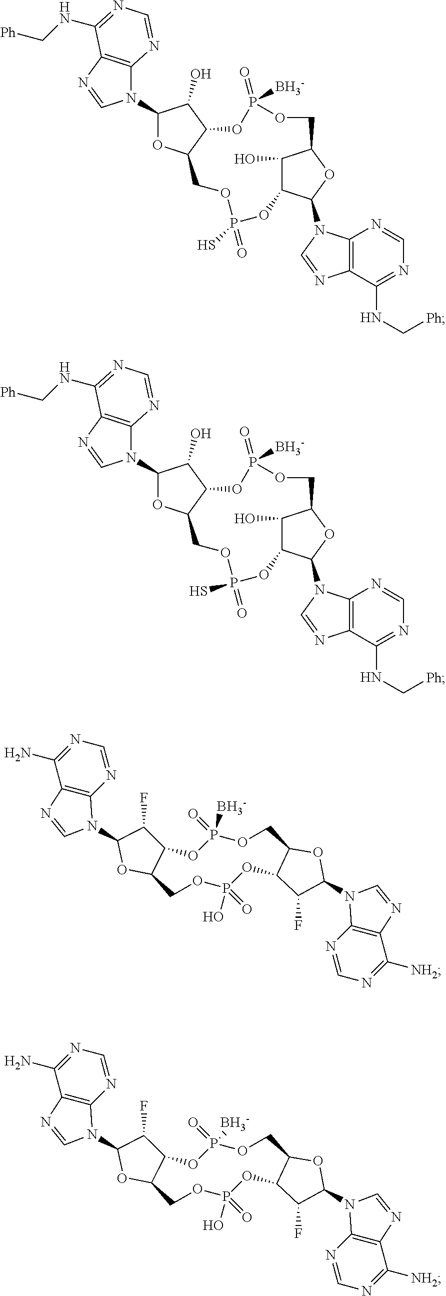

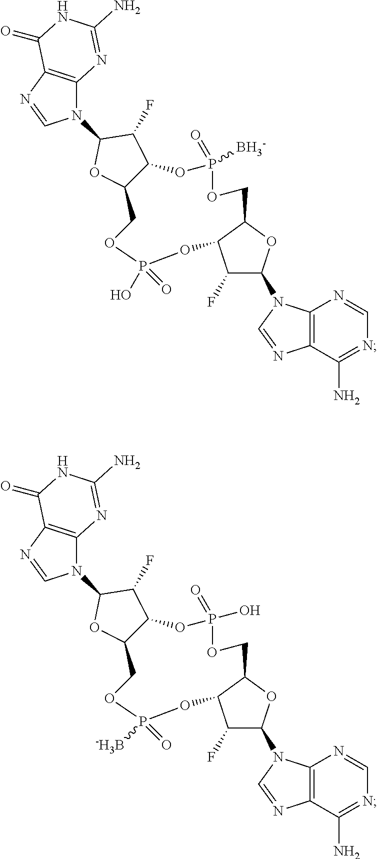

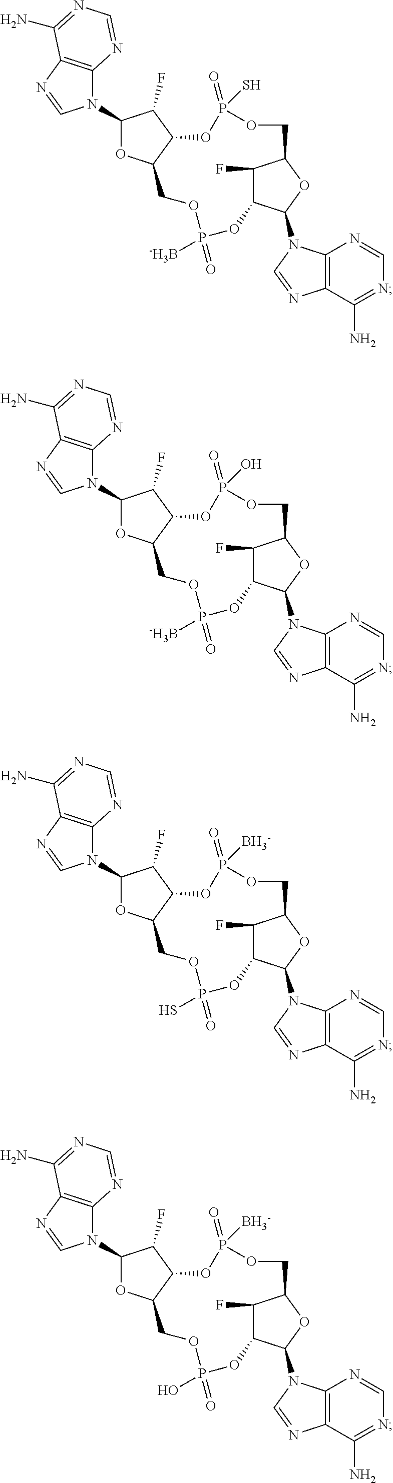

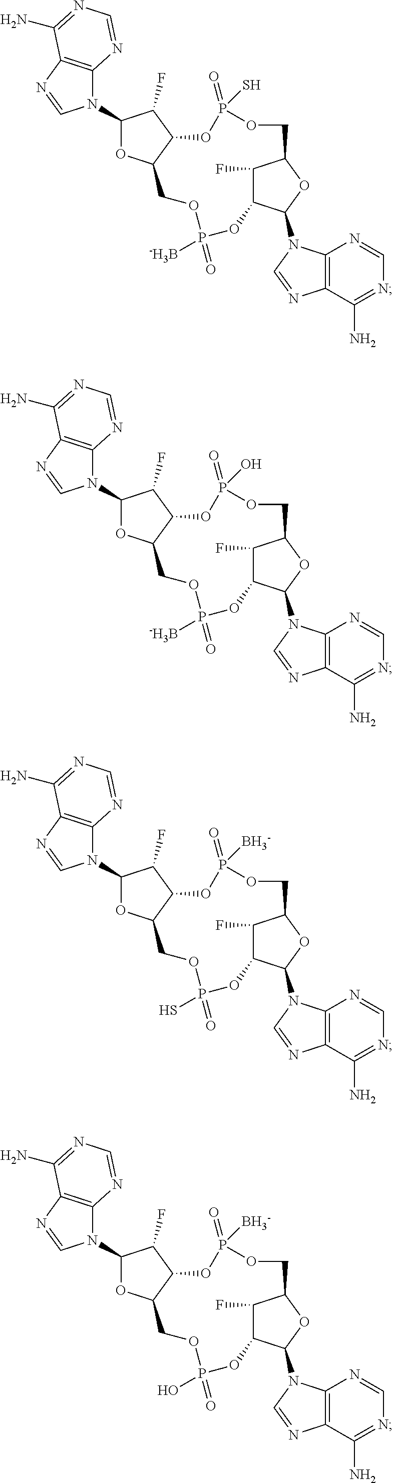

or a stereoisomer thereof.

In certain embodiments of the conjugate of formulae (XI), (XI-A), (XI-B), (XII), (XII-A), (XII-B), (XIII), (XIII-A), (XIII-B), (XIV), (XIV-A), (XIV-B), (XV), (XV-A), (XV-B), (XXVI), (XXVI-A), (XXVI-B), (XXVII), (XXVII-A) and (XXVII-B), the cyclic dinucleotide is:

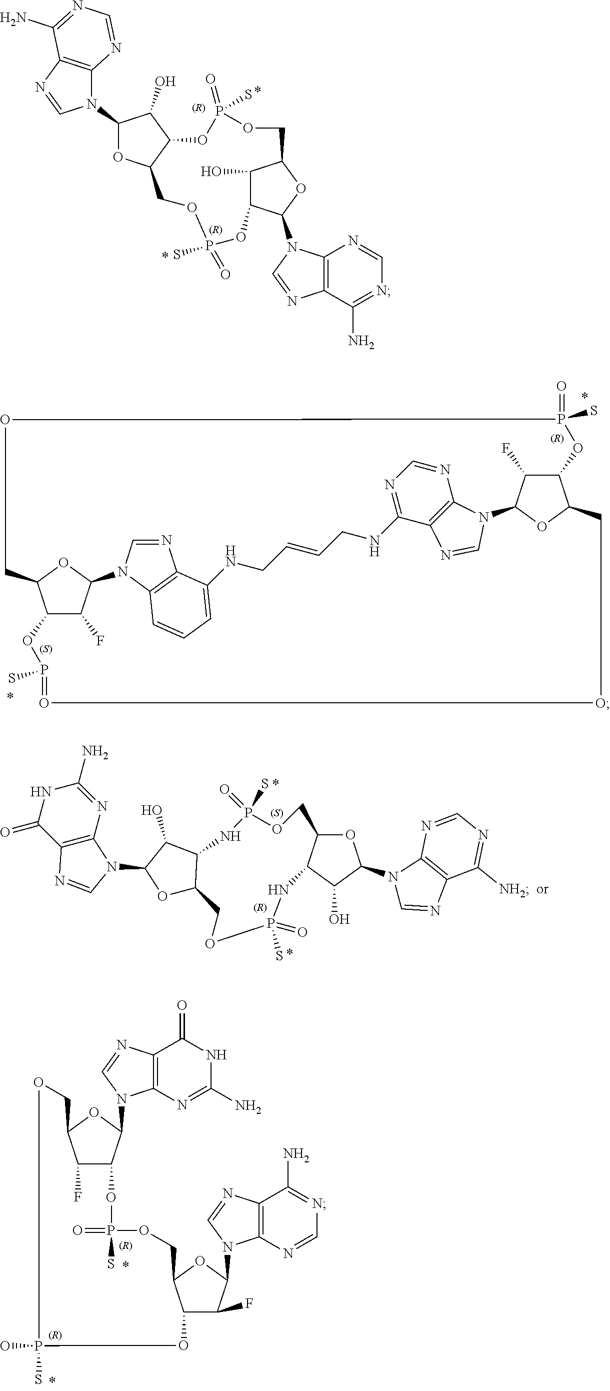

or a stereoisomer thereof;
   wherein the * indicates the S of thiol group (—SH, H not shown where * appears) of the cyclic dinucleotide that is connecting with the linker. In certain embodiments, when b is 1, one S* of the cyclic dinucleotide is connected to a linker and the other S* is connected to a hydrogen. In certain embodiments, when b is 2, both S* of the cyclic dinucleotide are connected to a linker.

In certain embodiments, the conjugate is selected from the group consisting of:

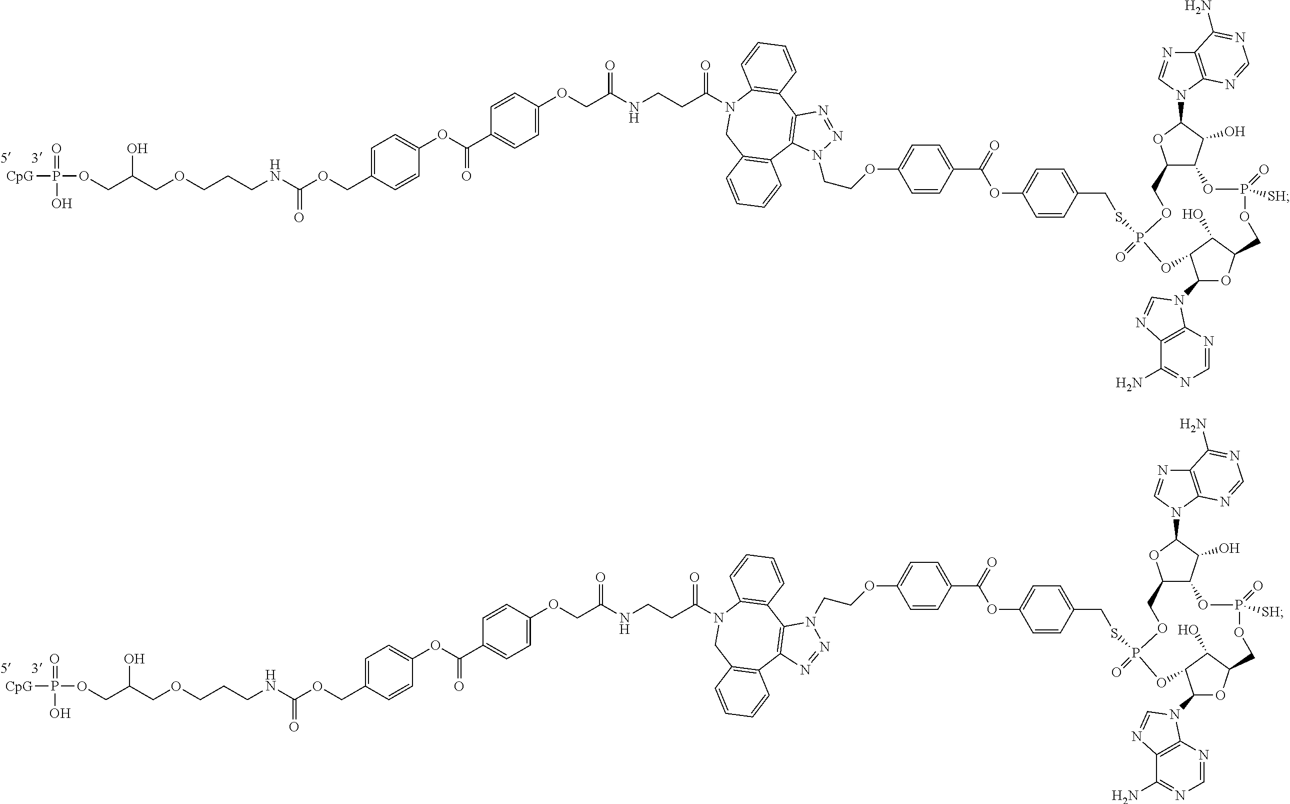

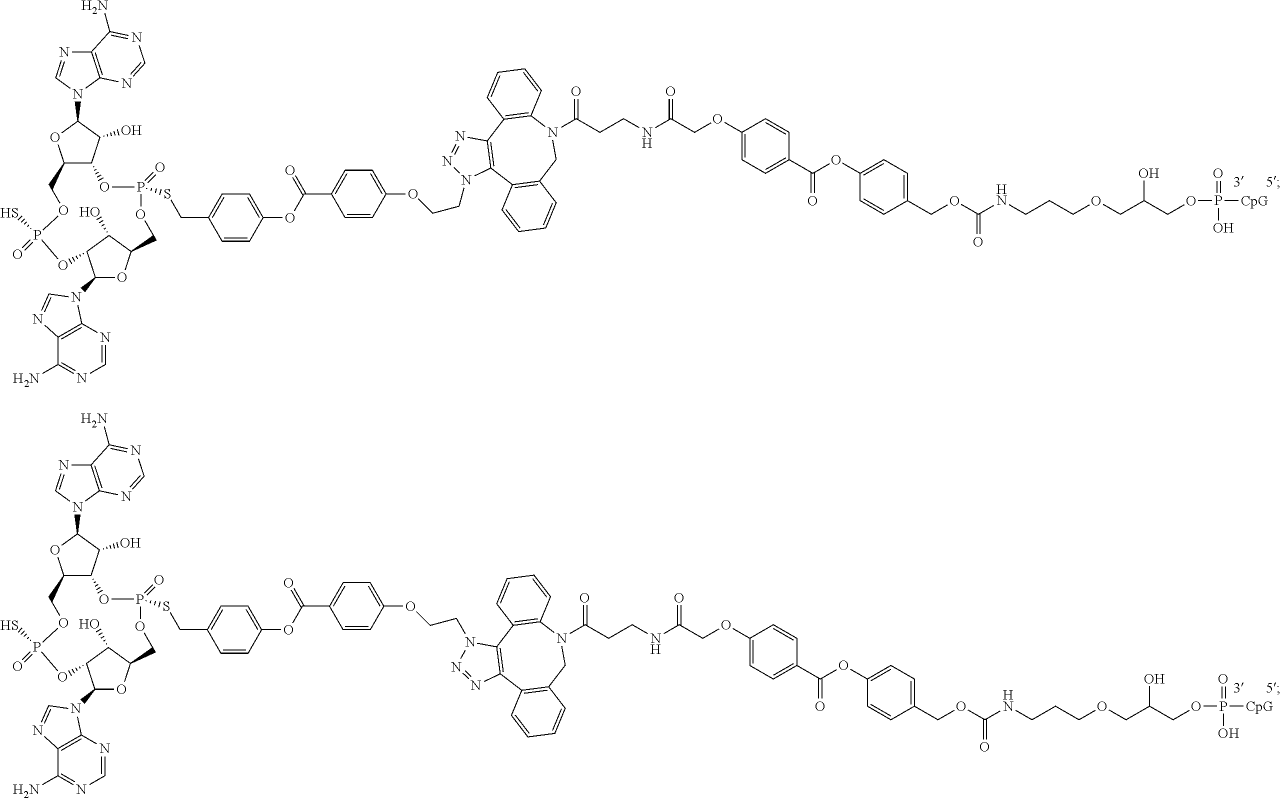

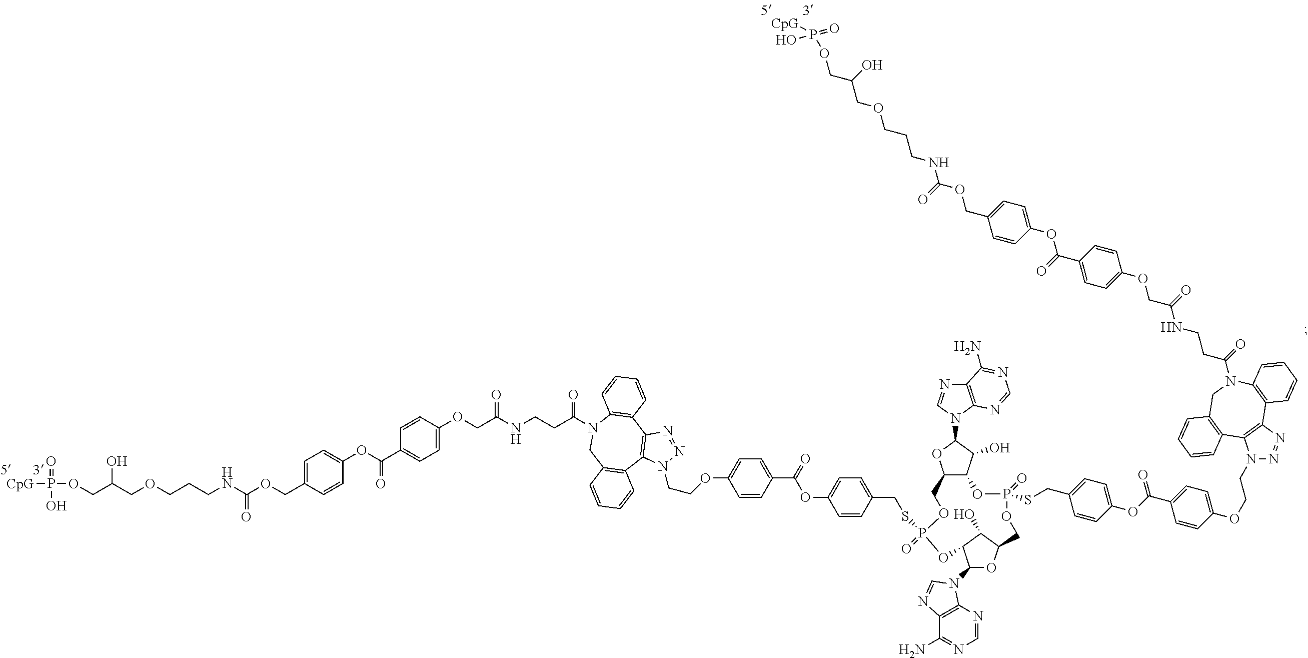

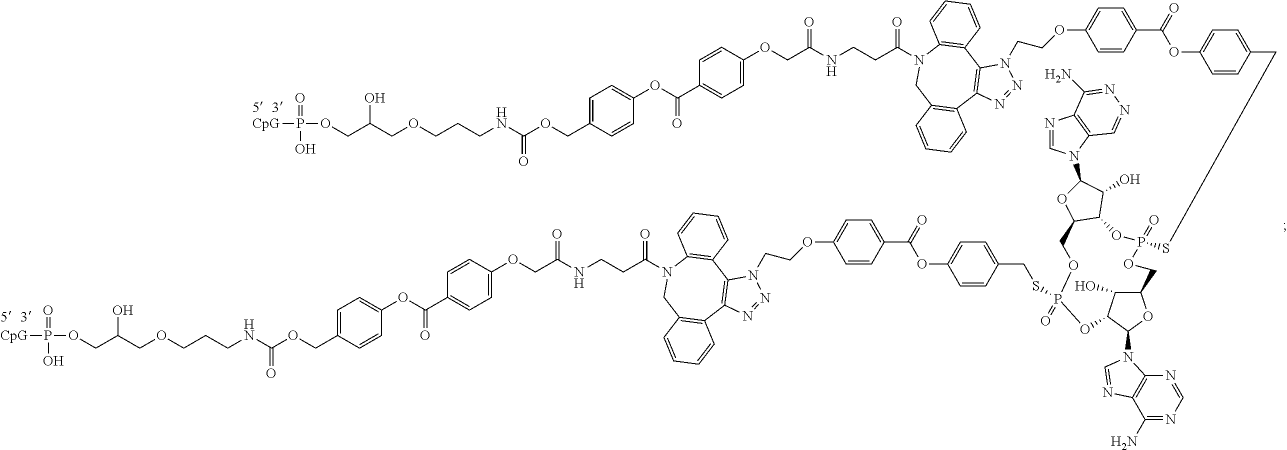

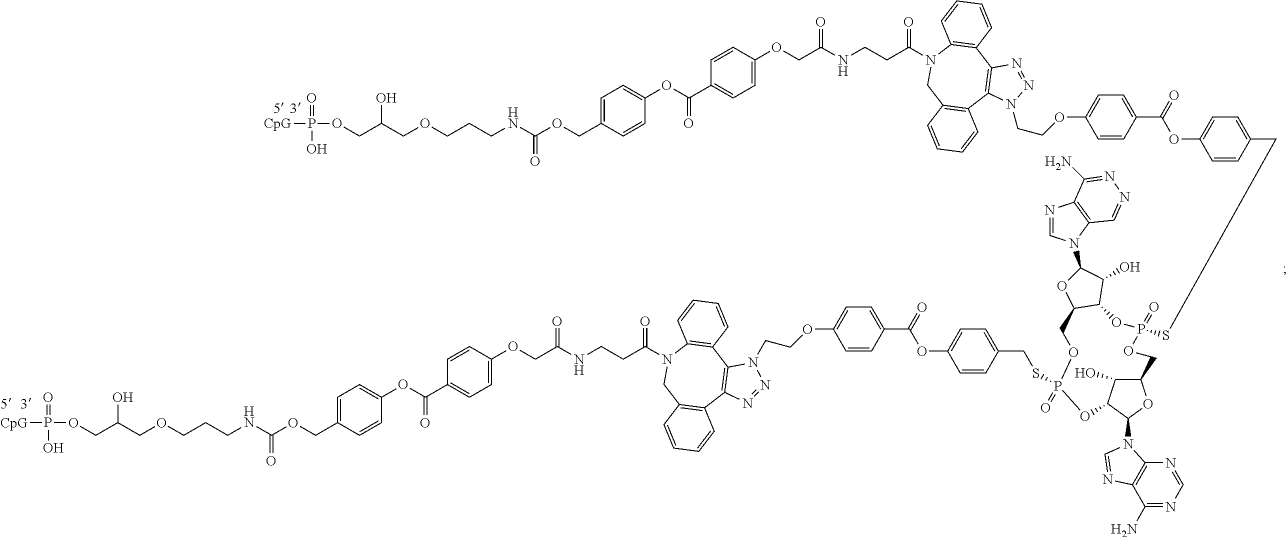

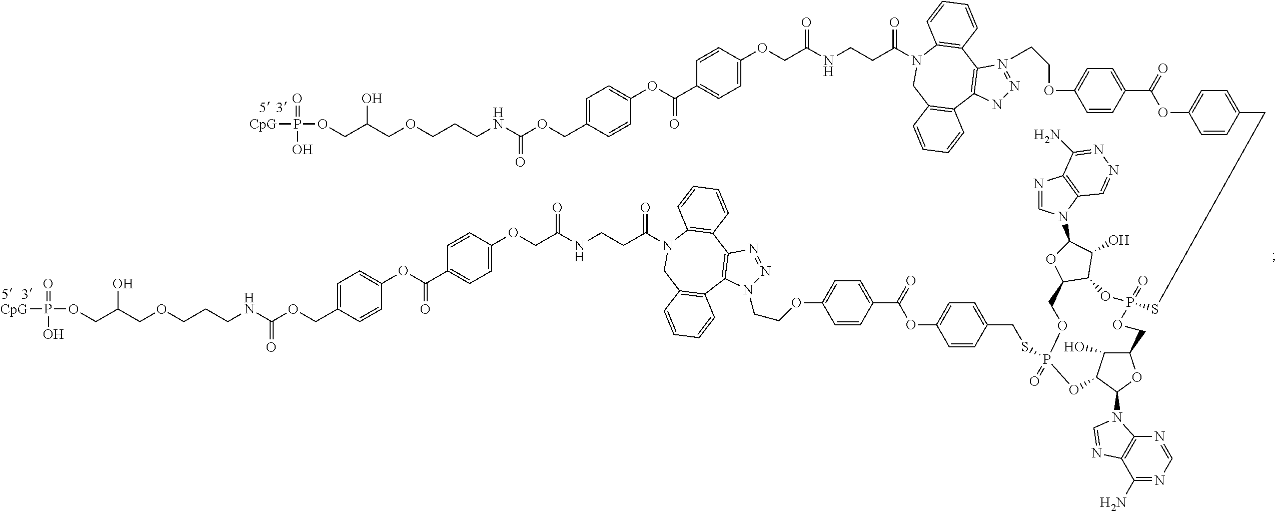

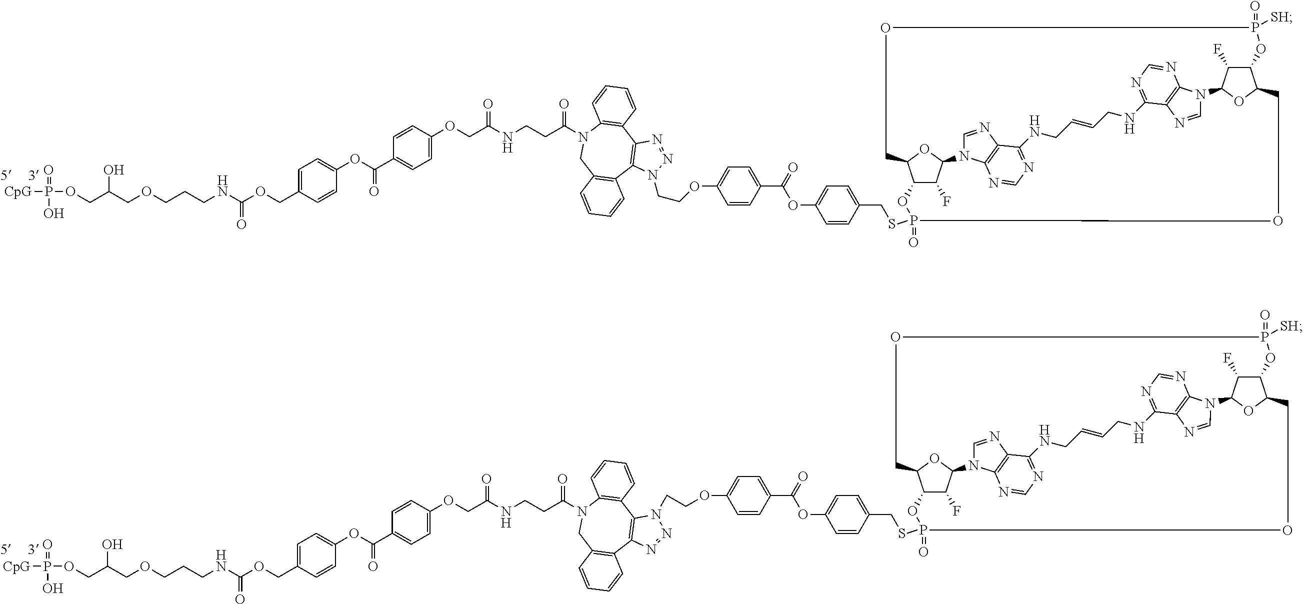

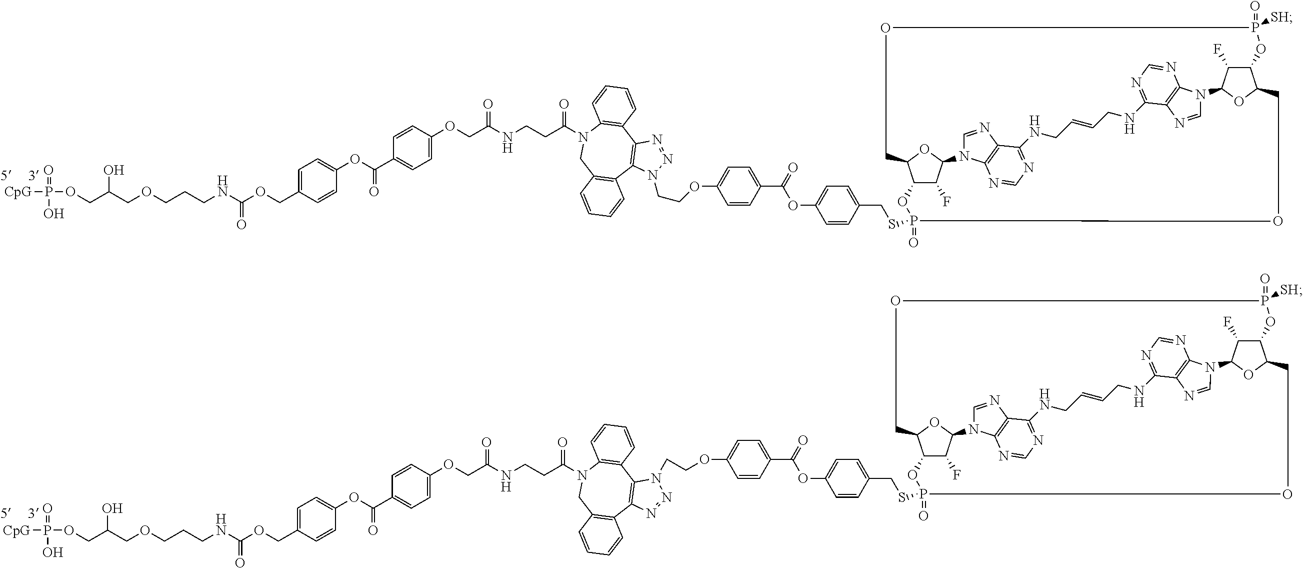

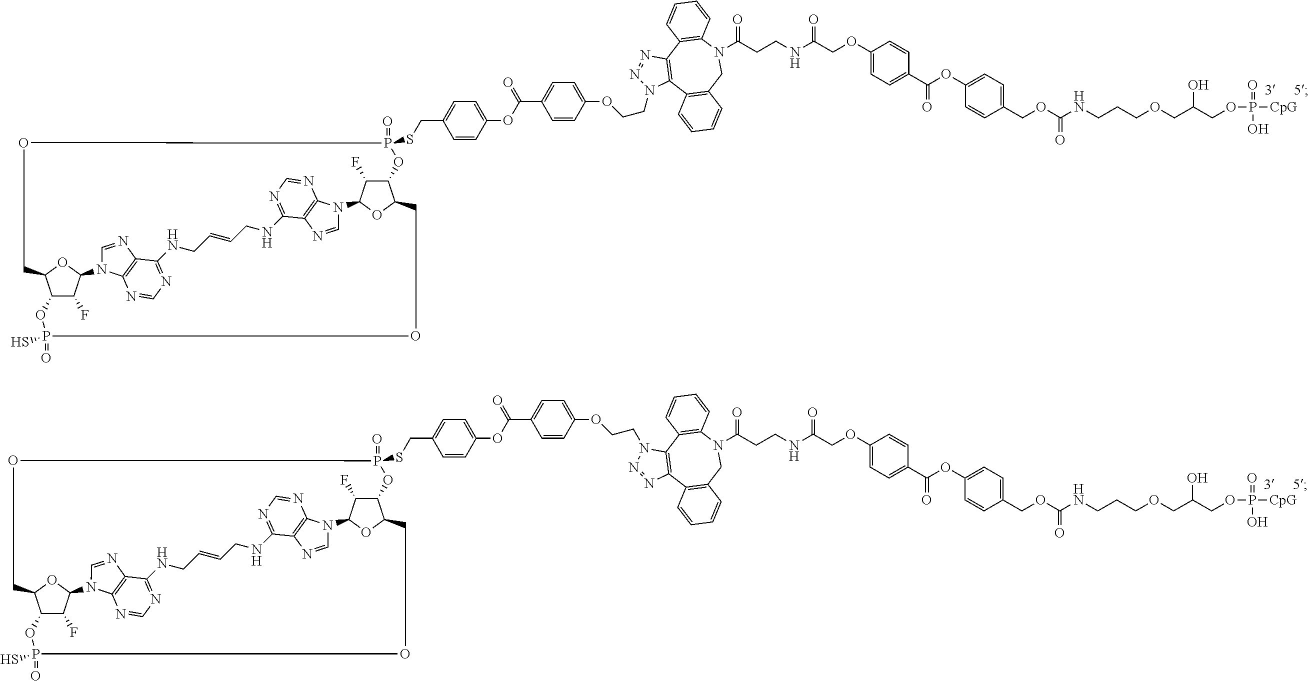

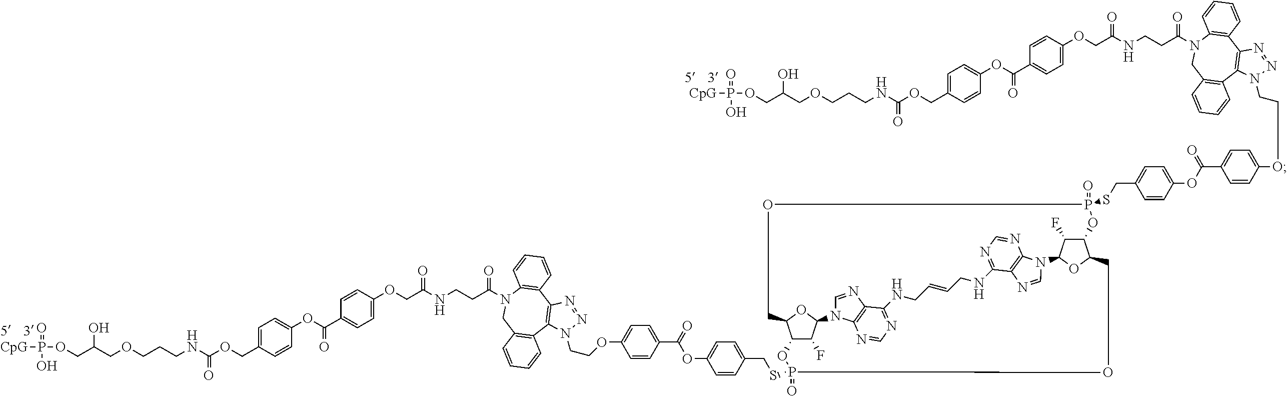

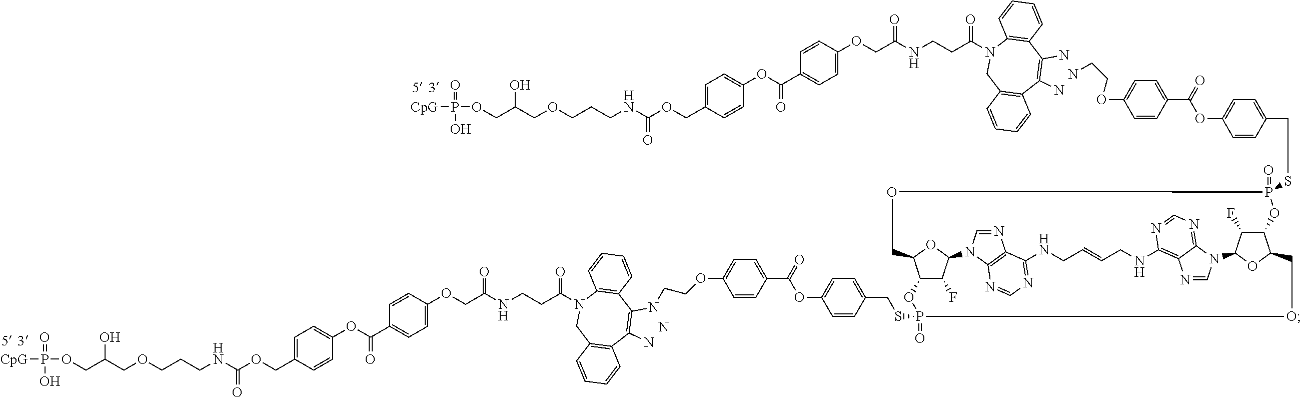

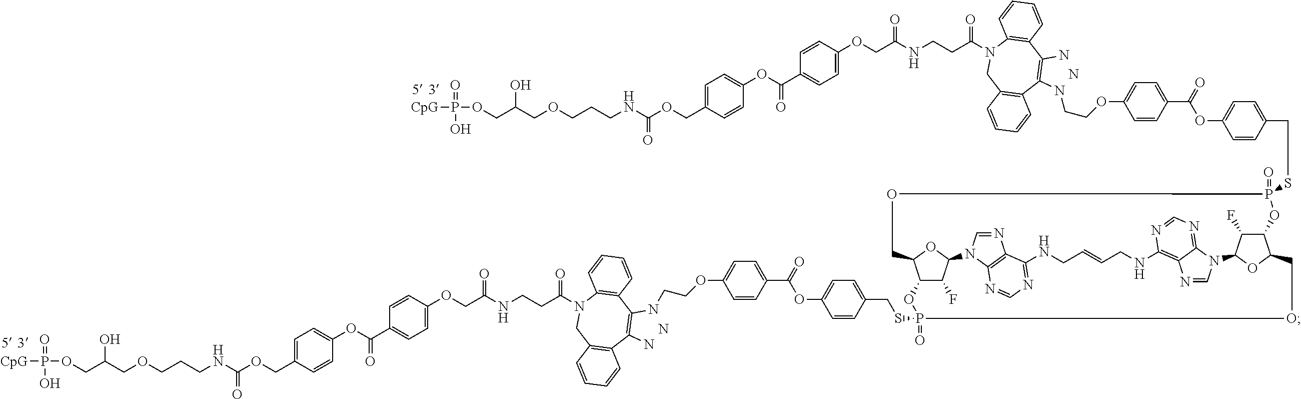

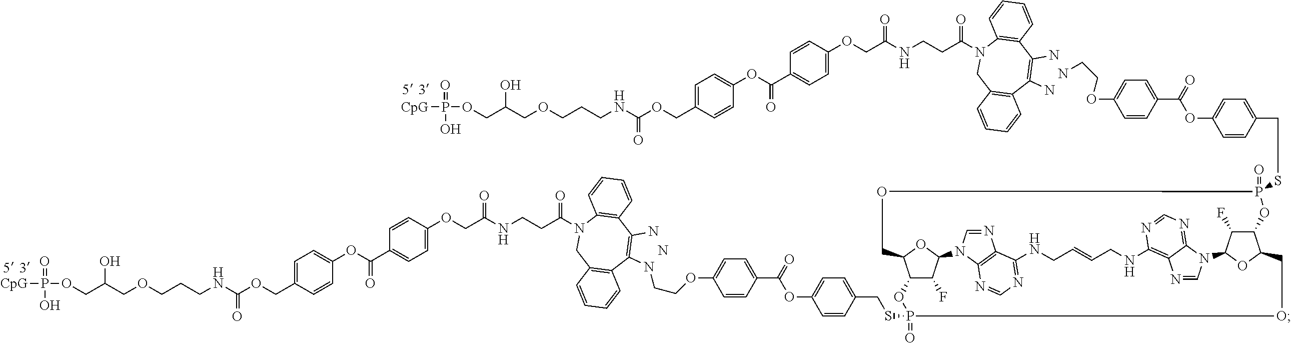

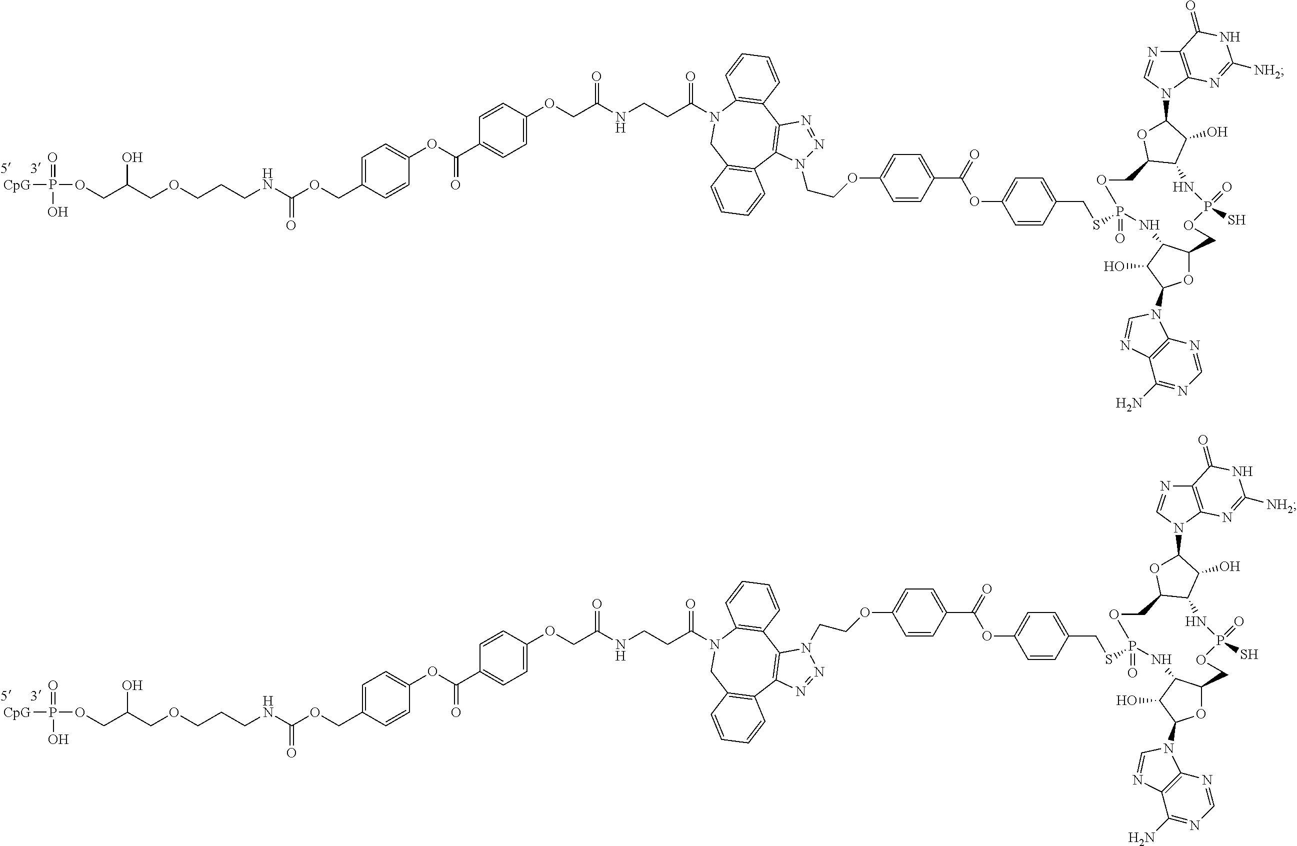

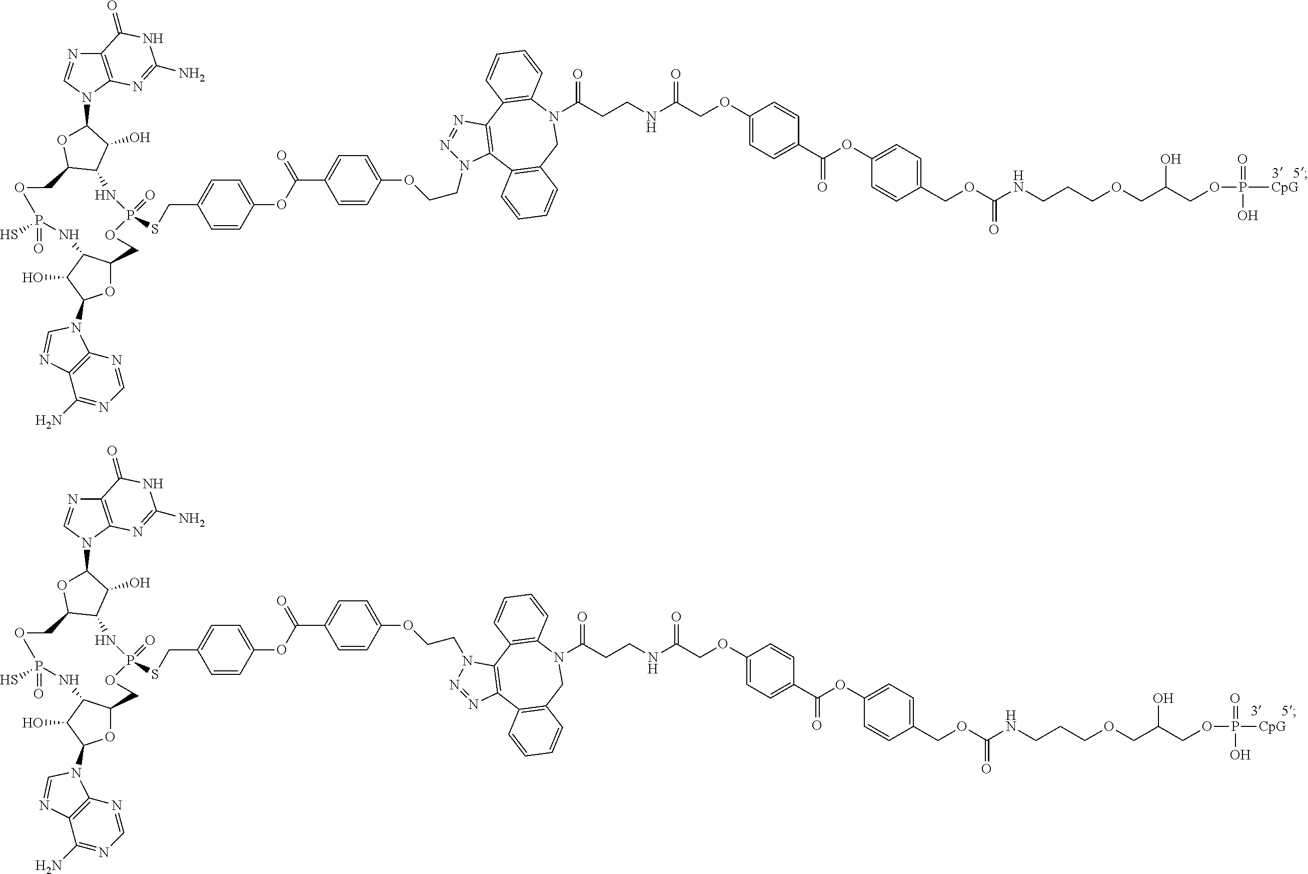

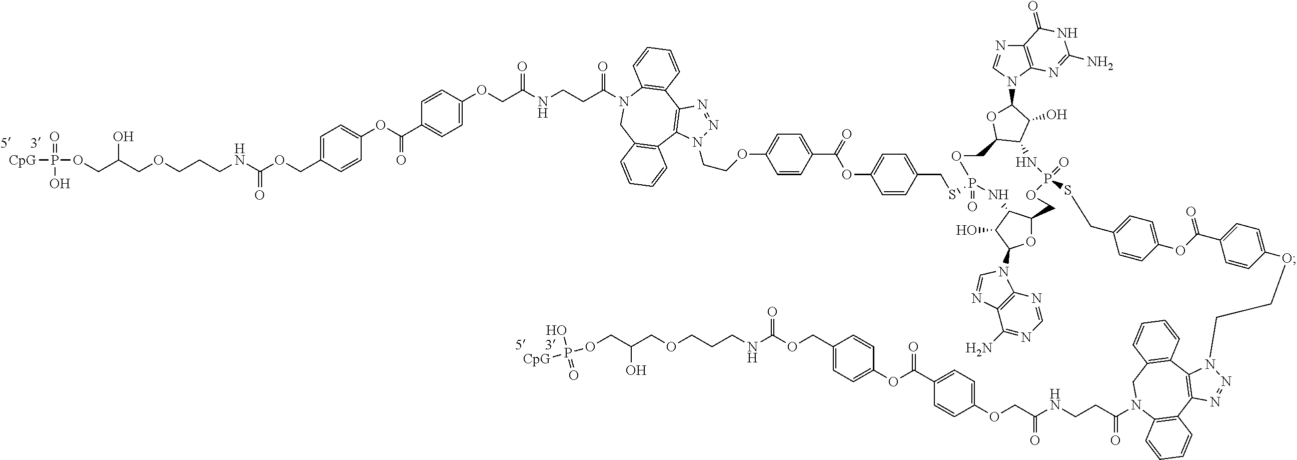

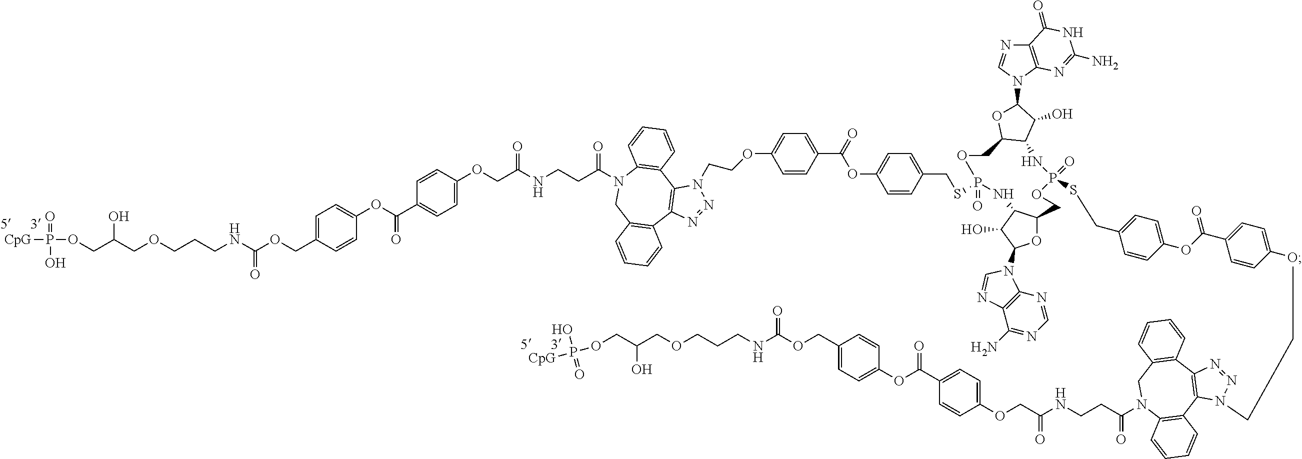

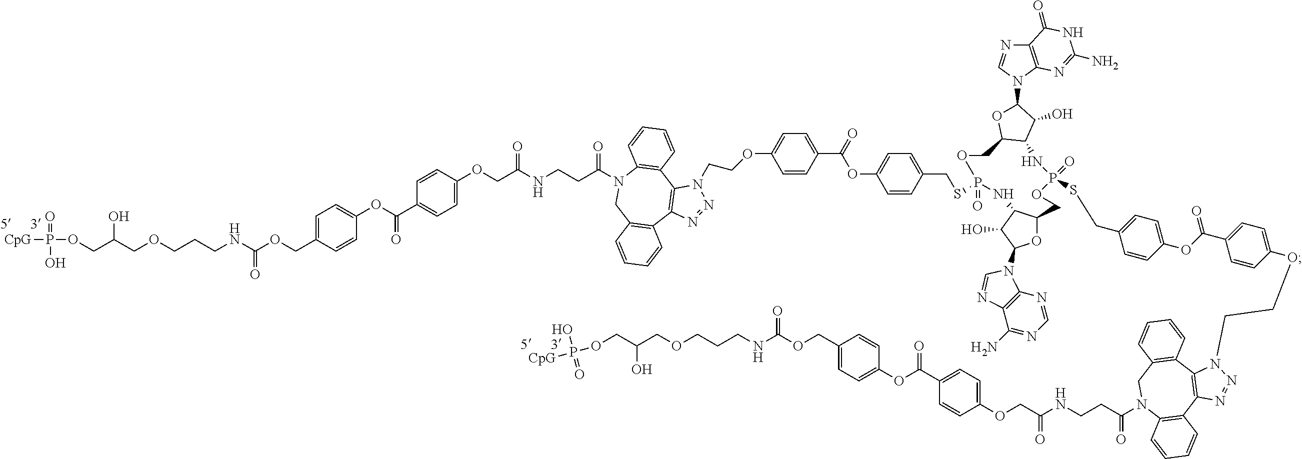

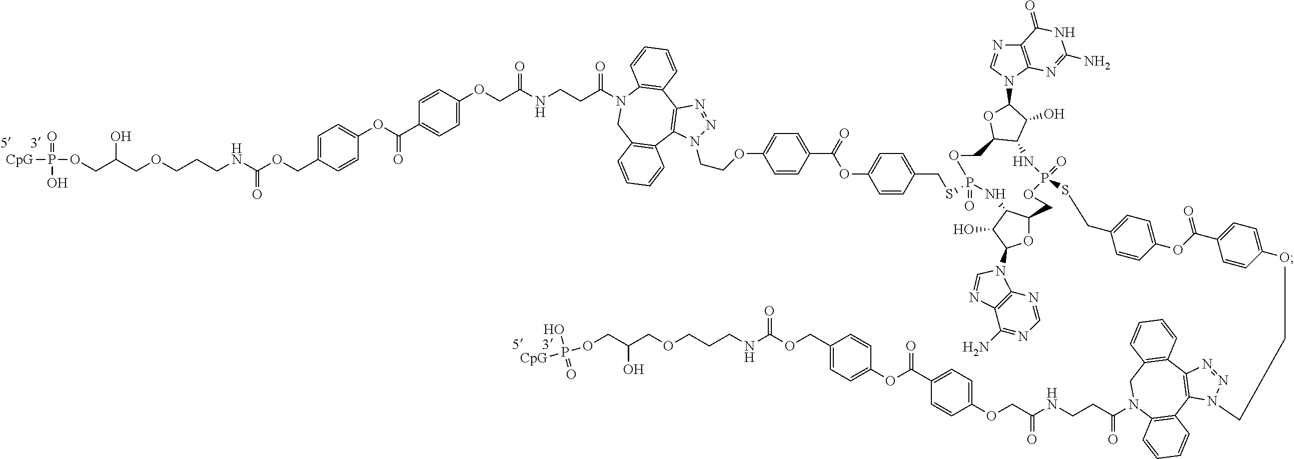

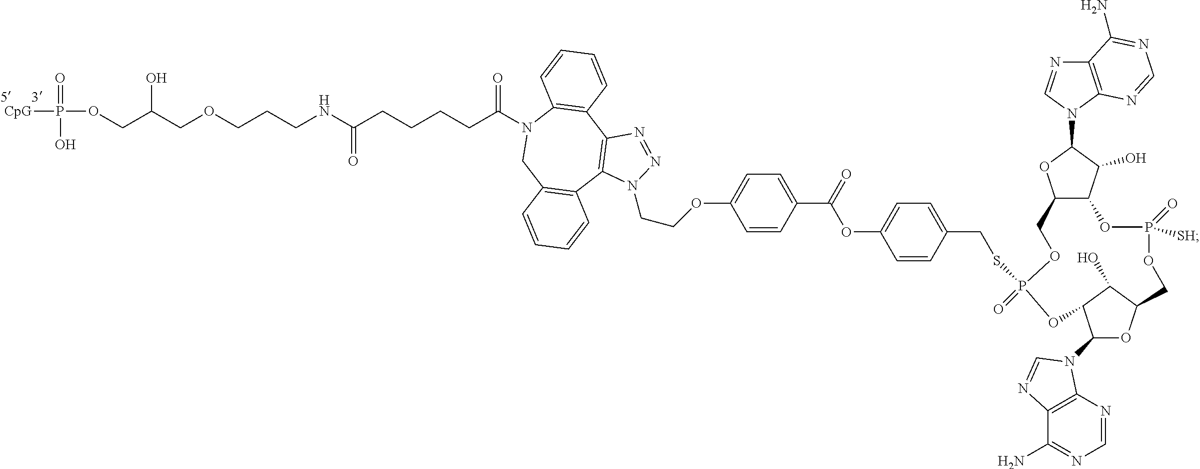

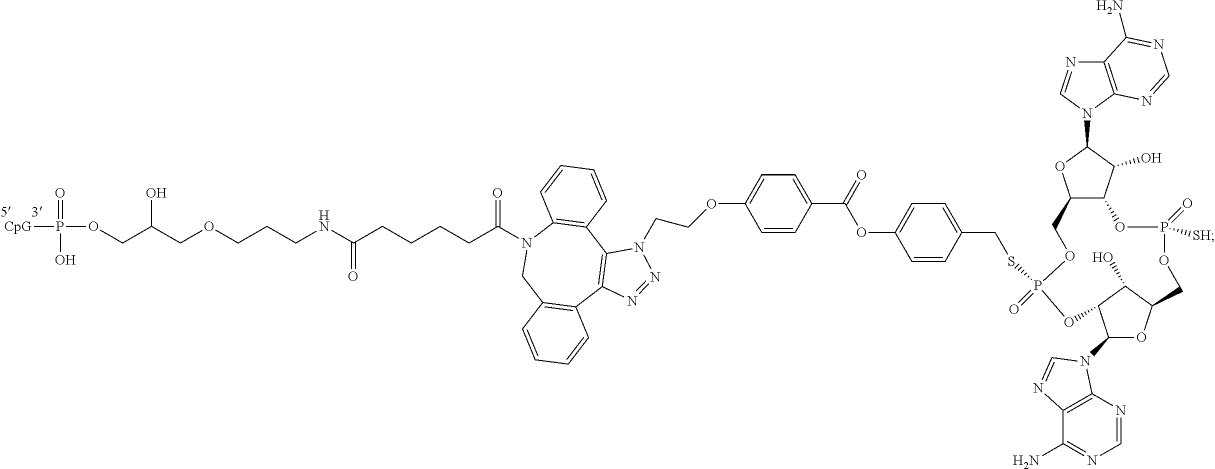

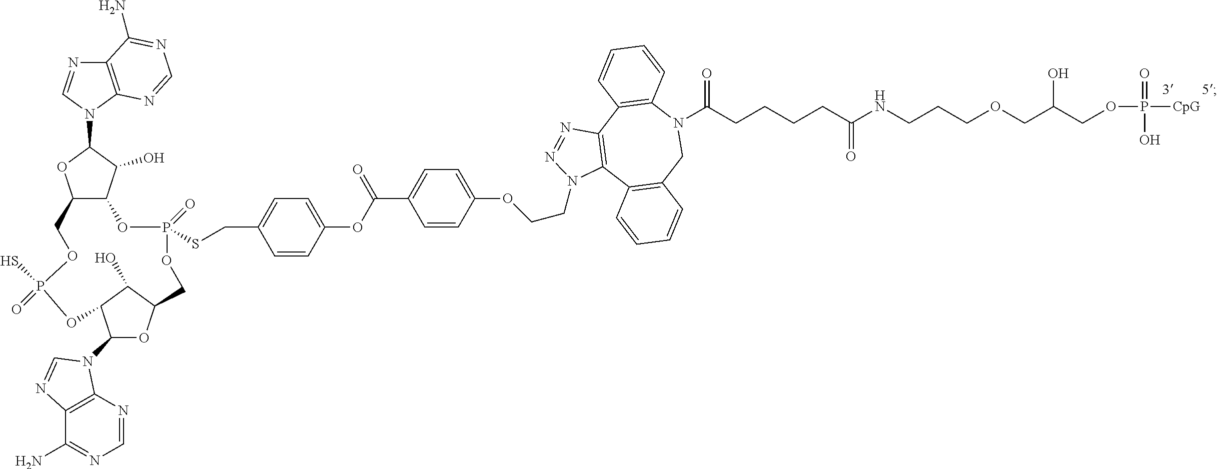

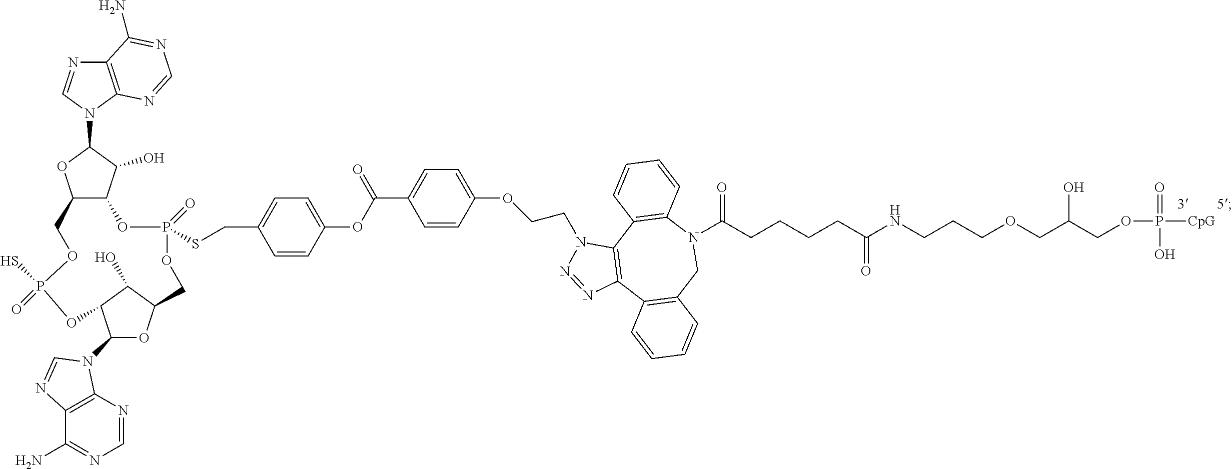

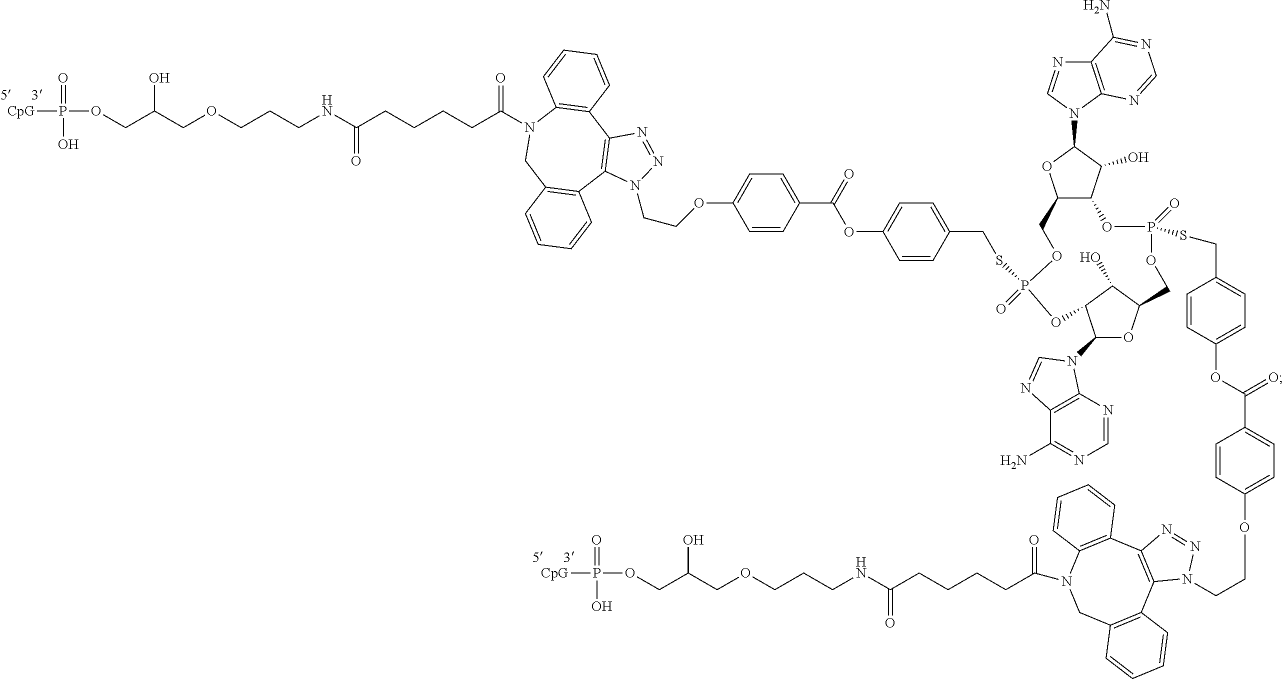

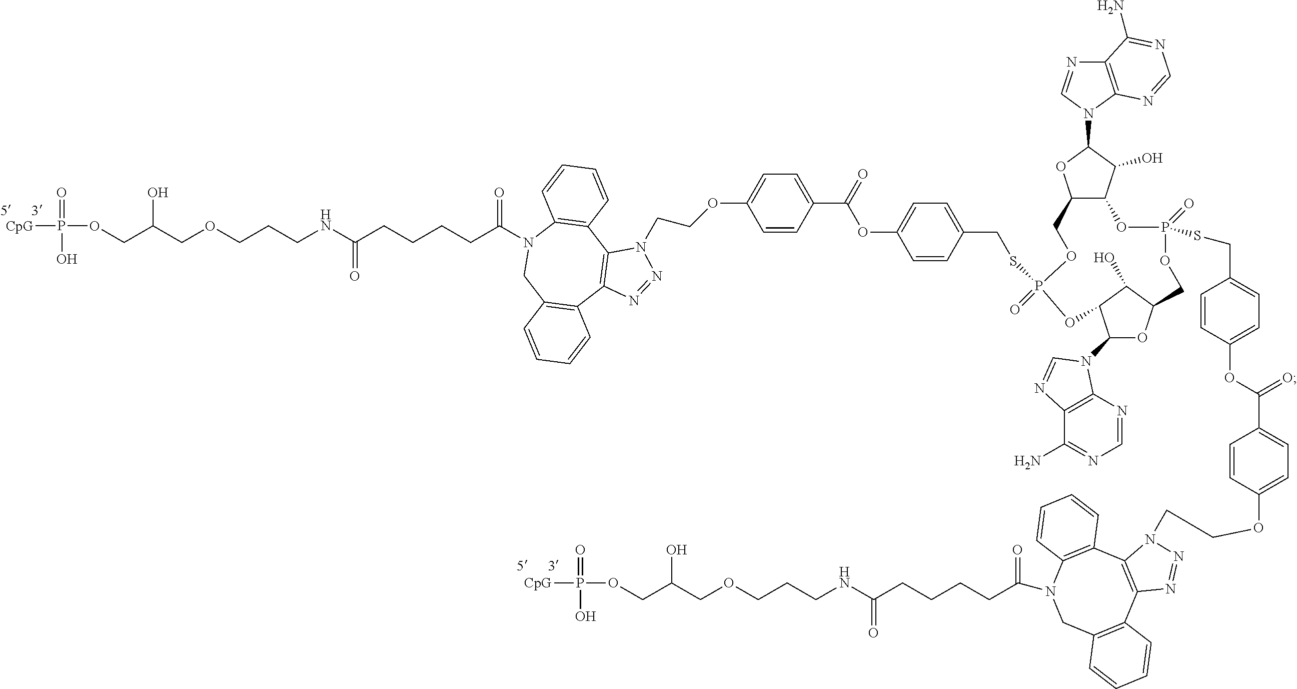

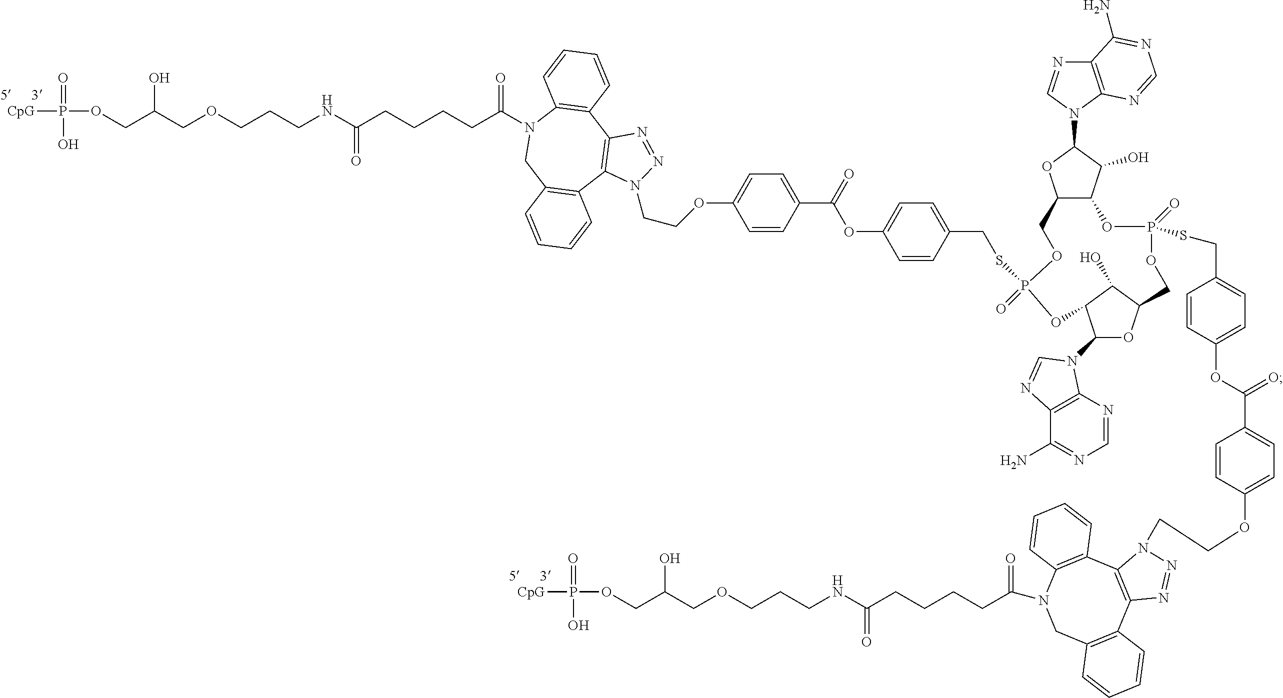

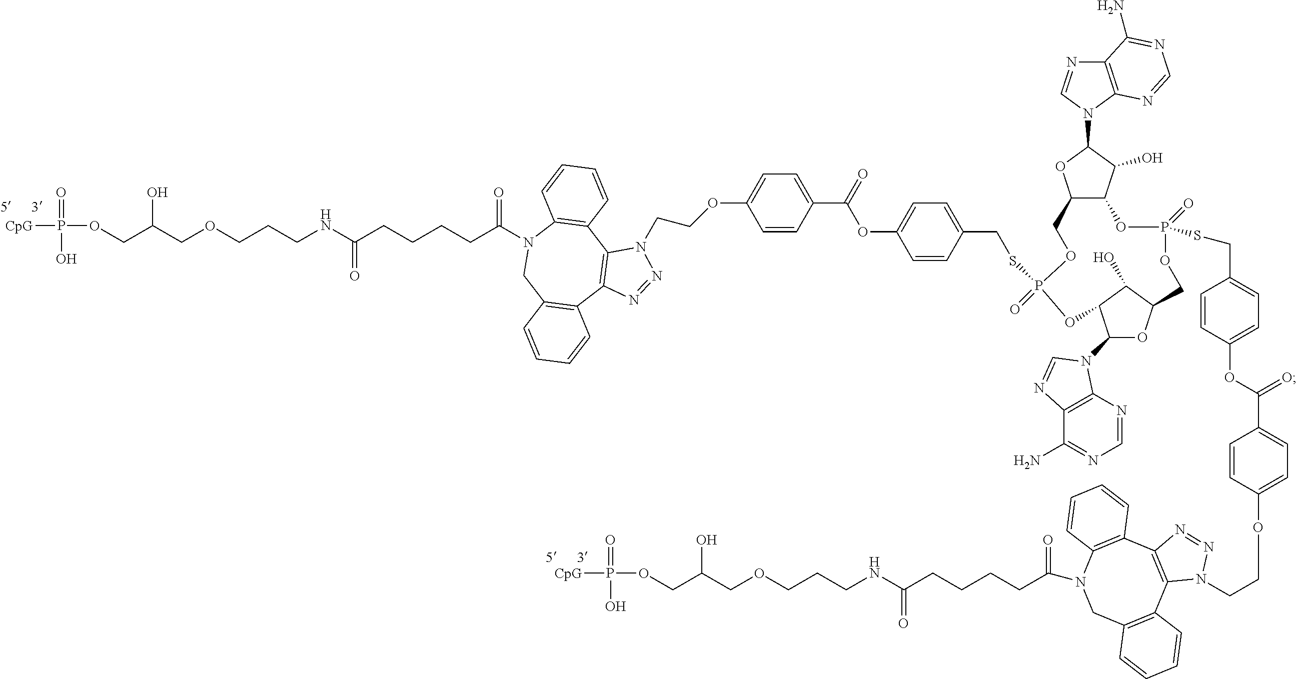

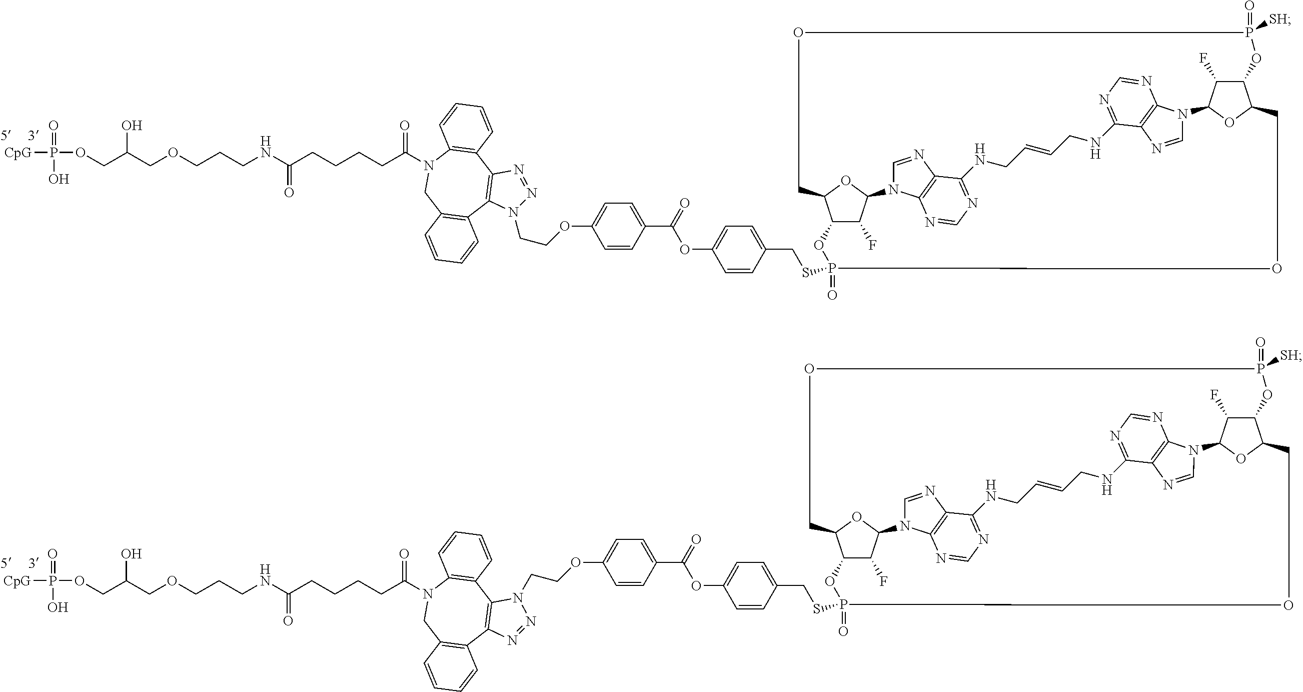

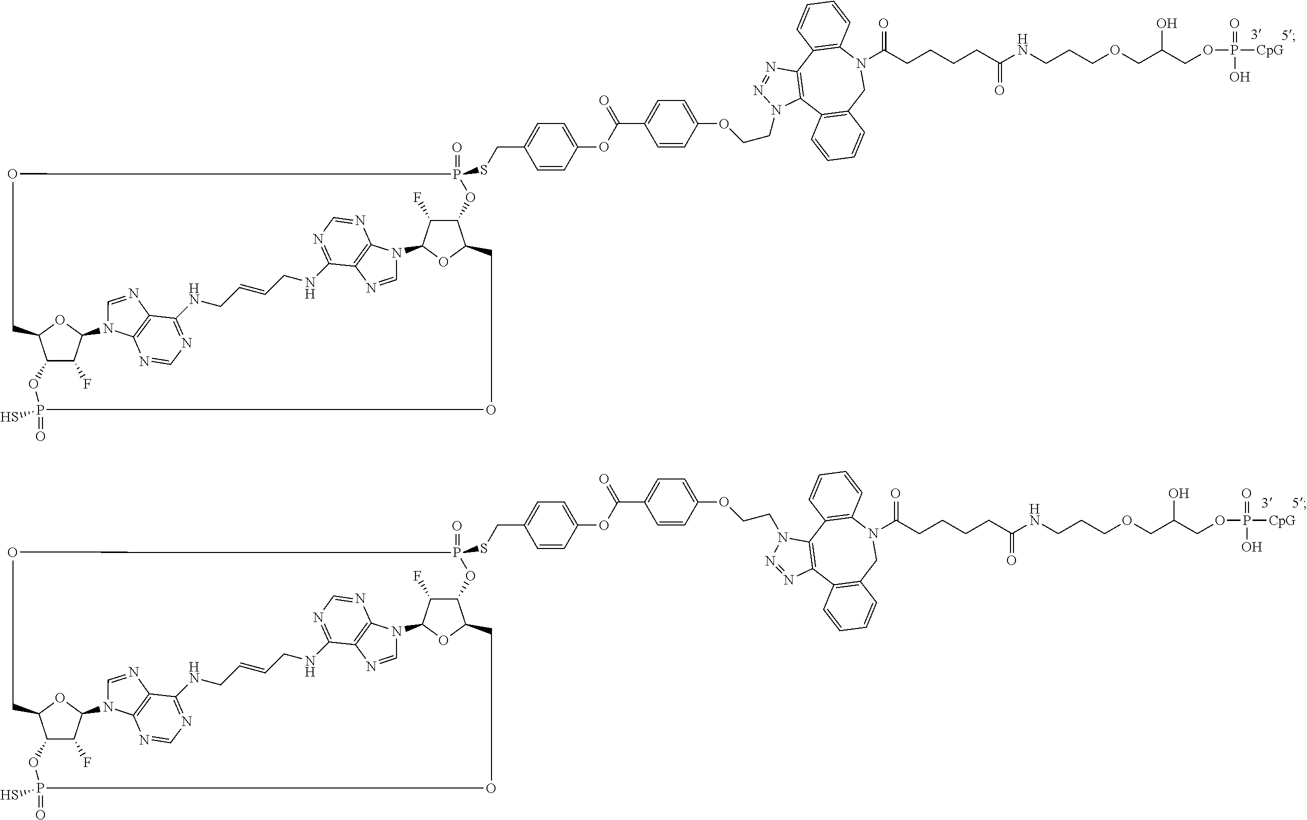

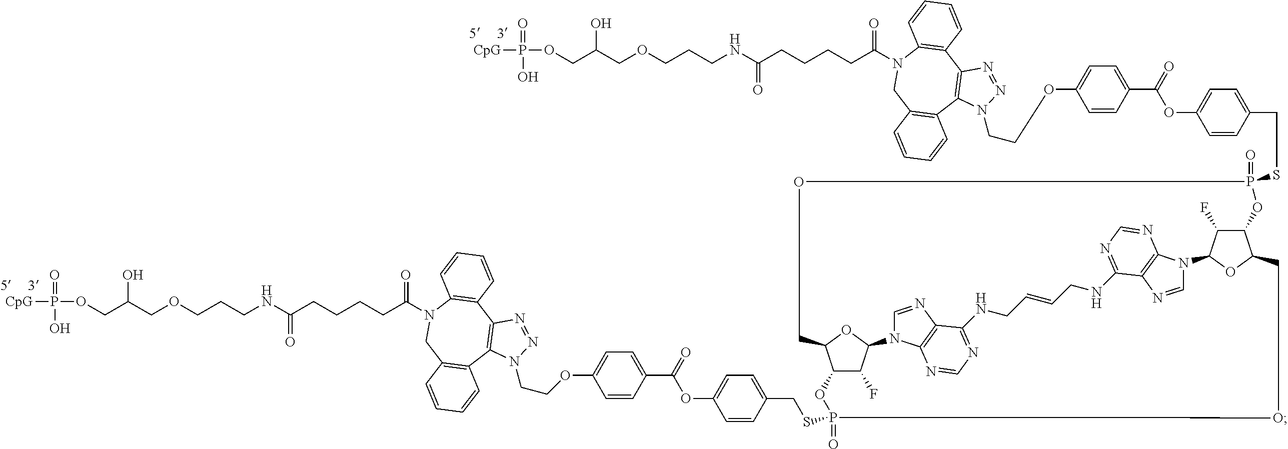

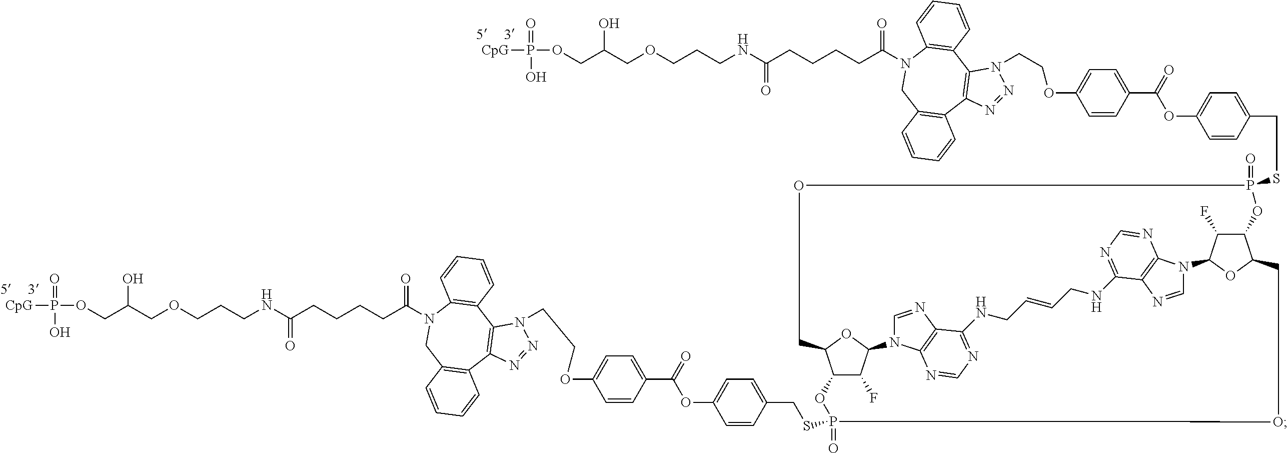

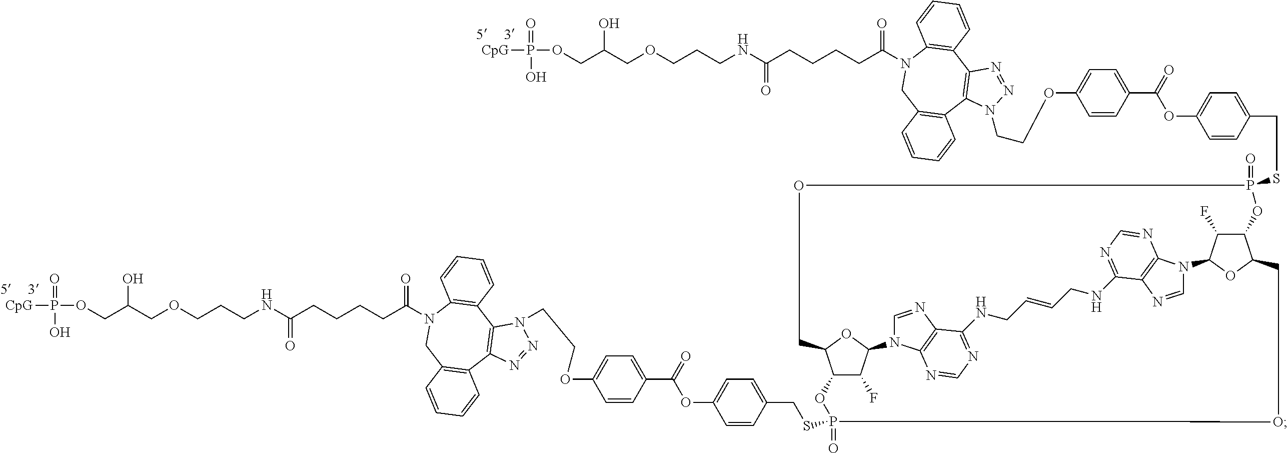

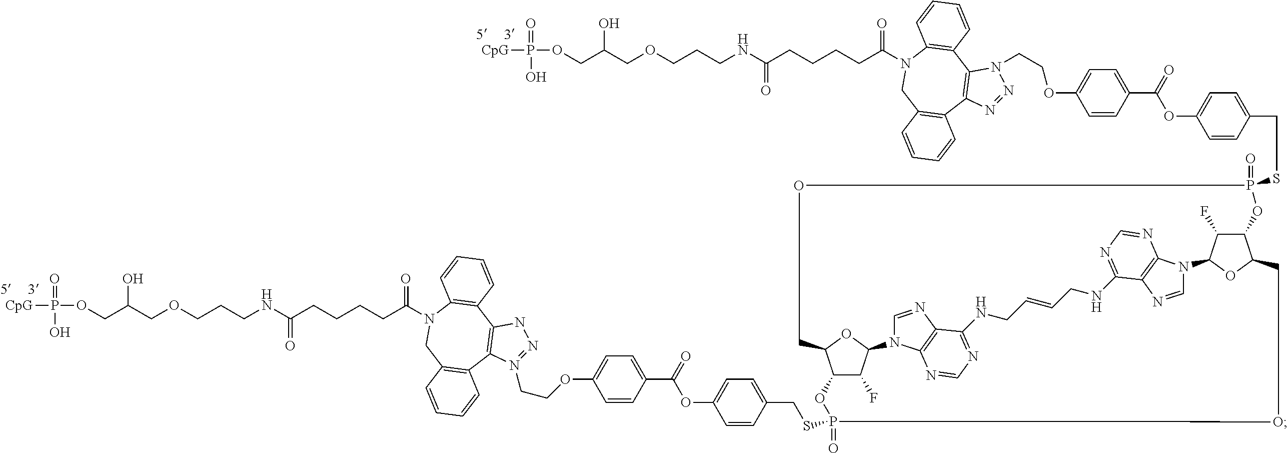

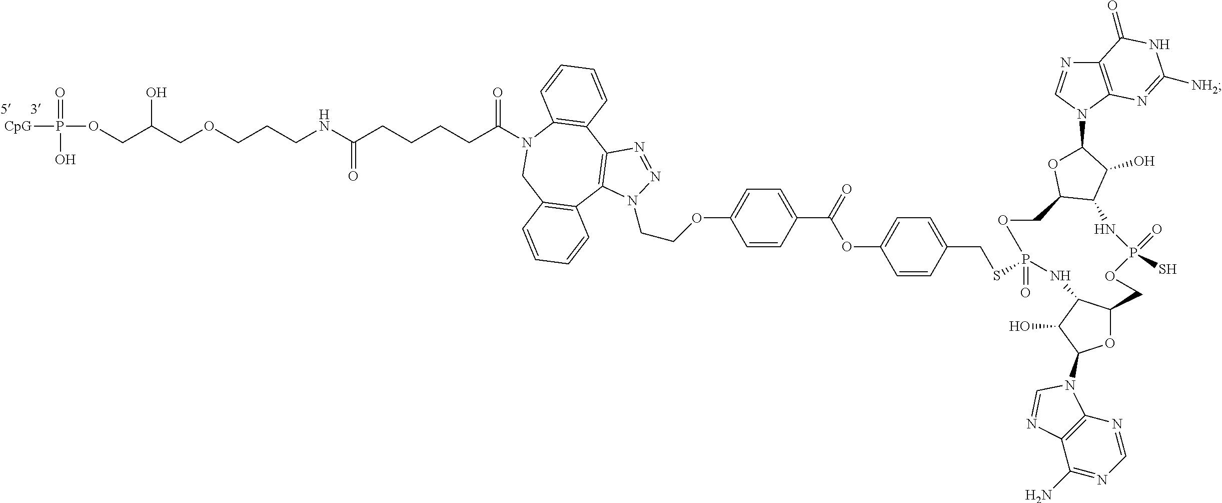

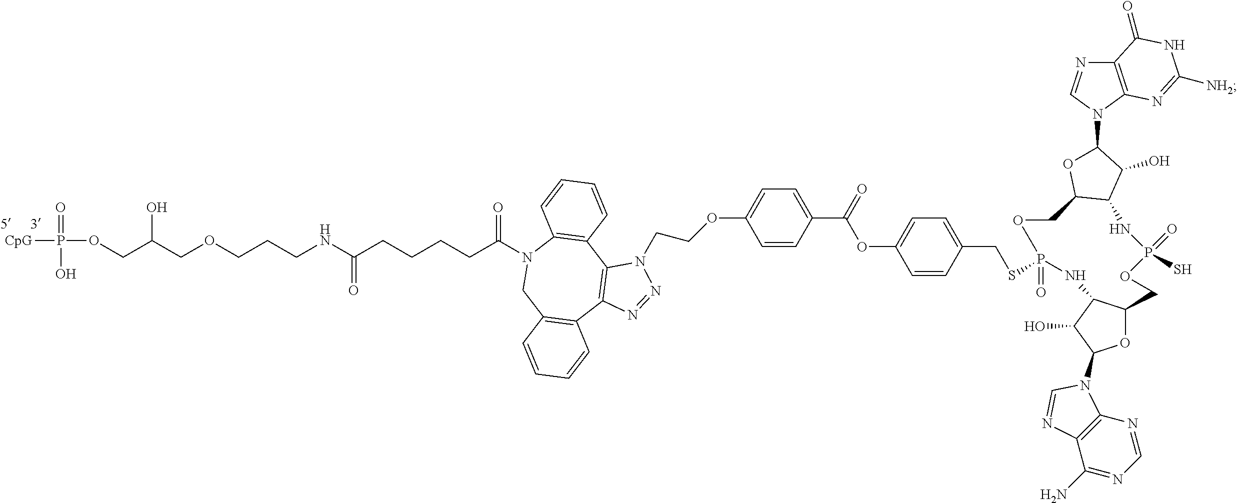

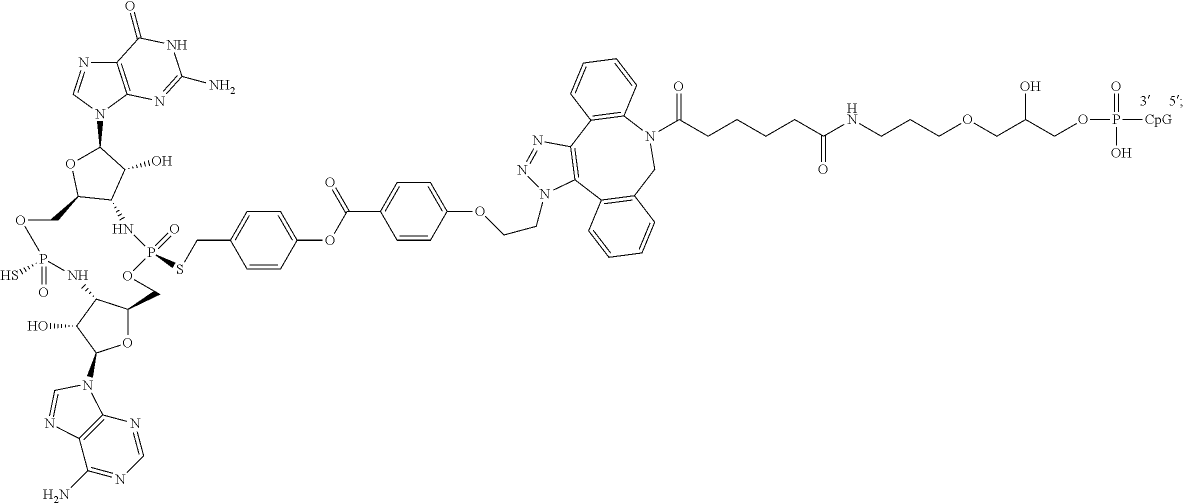

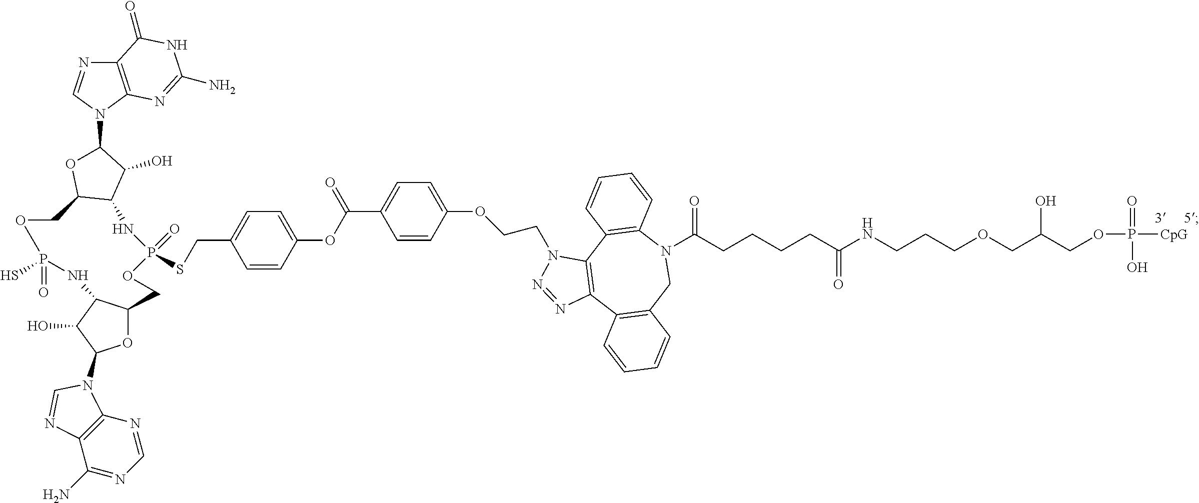

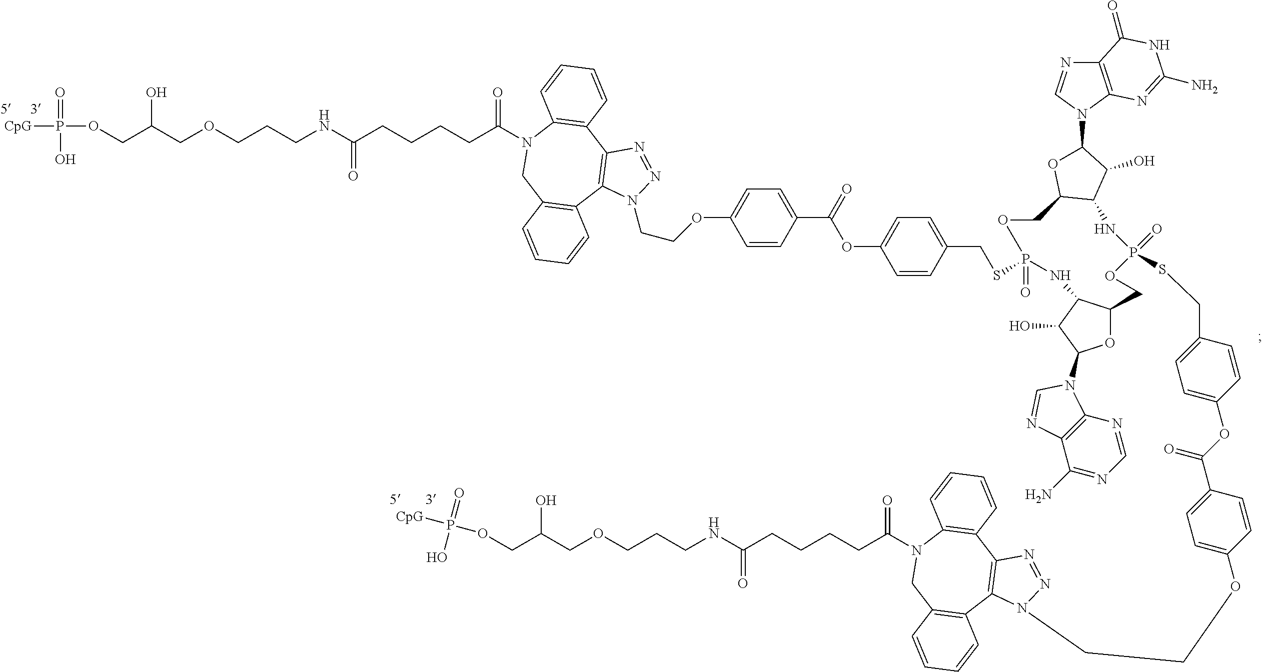

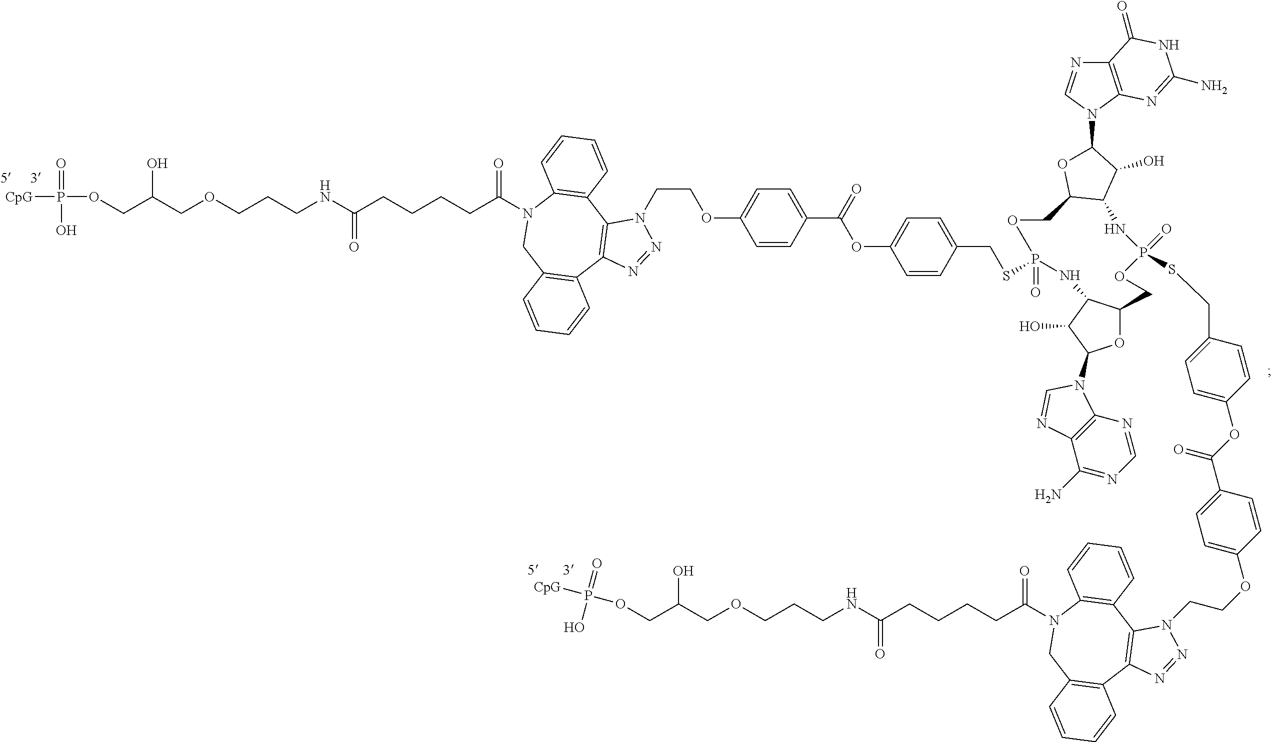

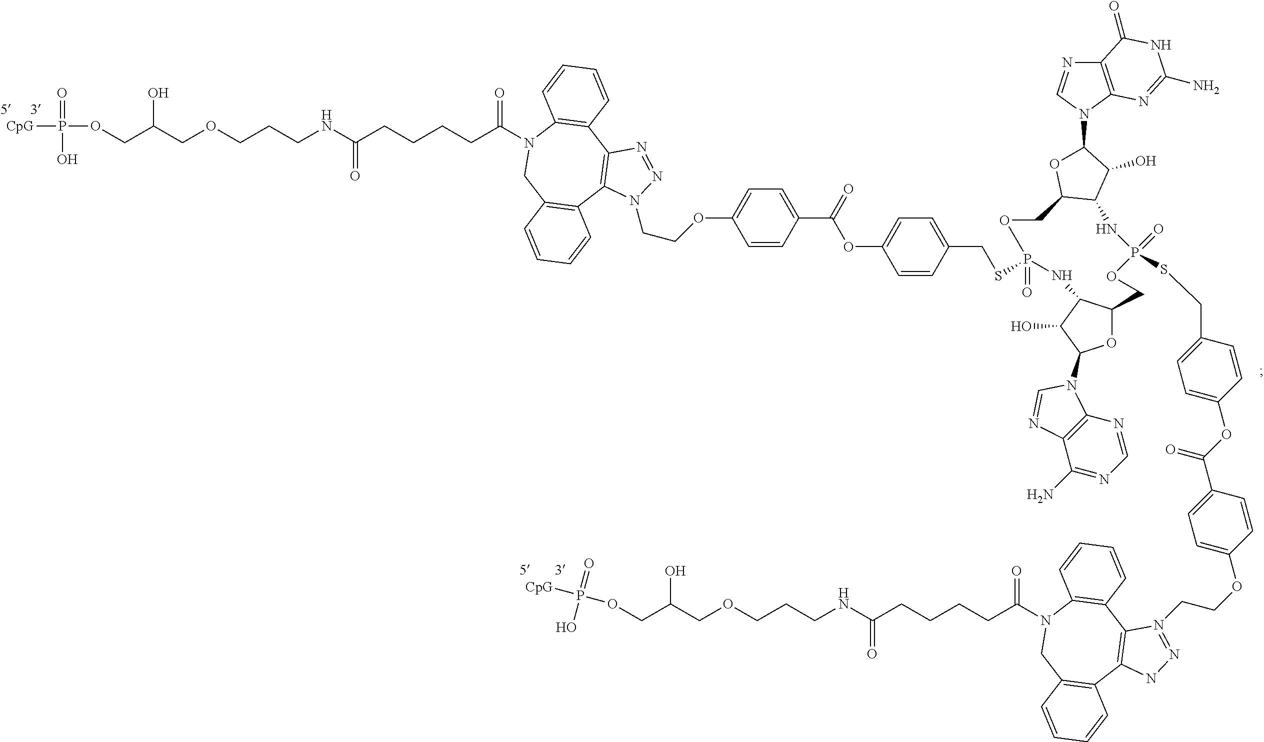

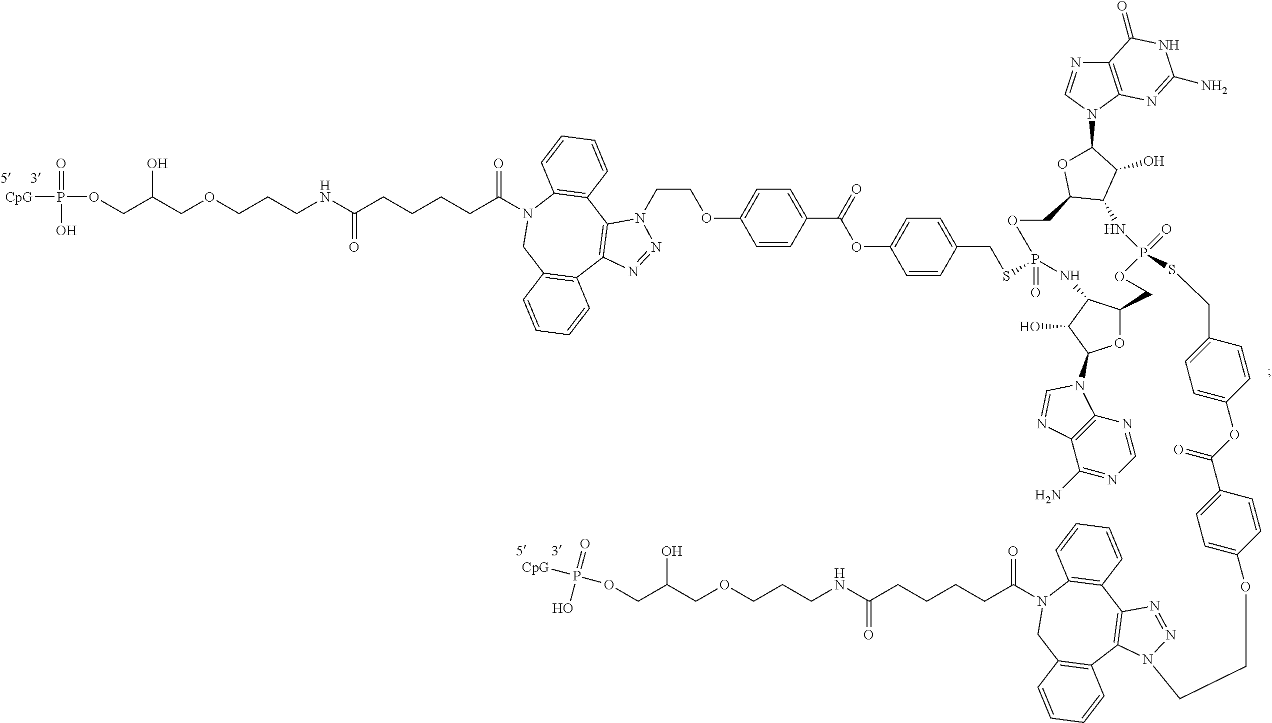

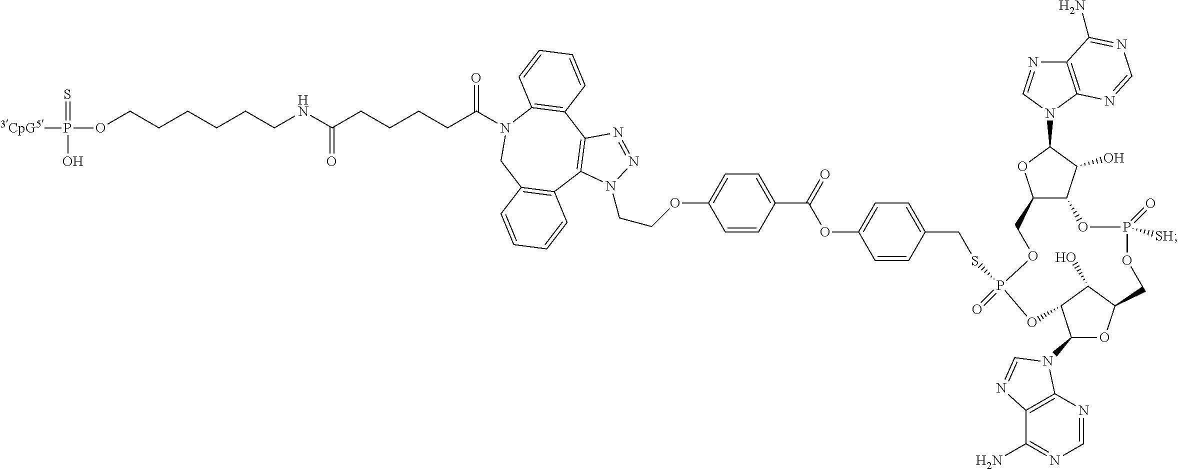

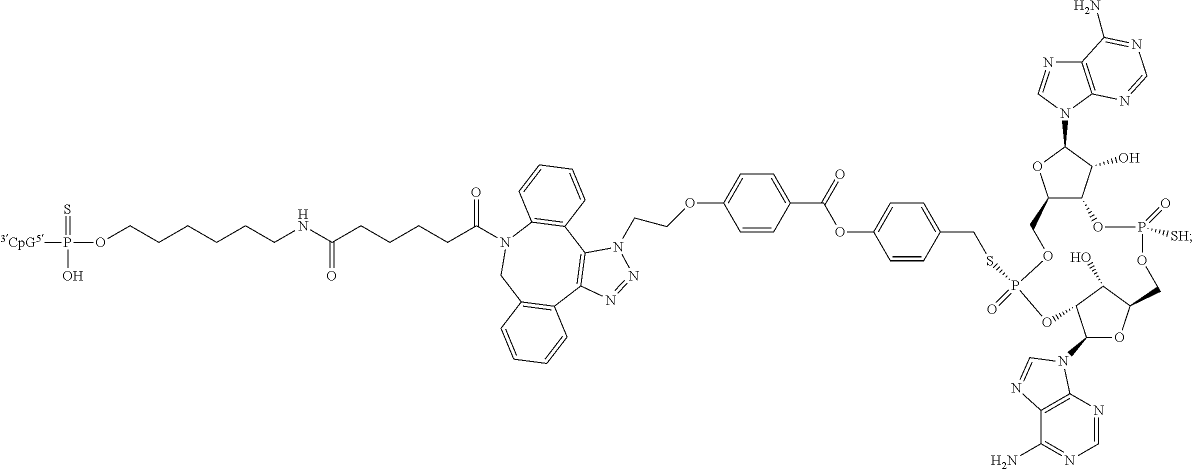

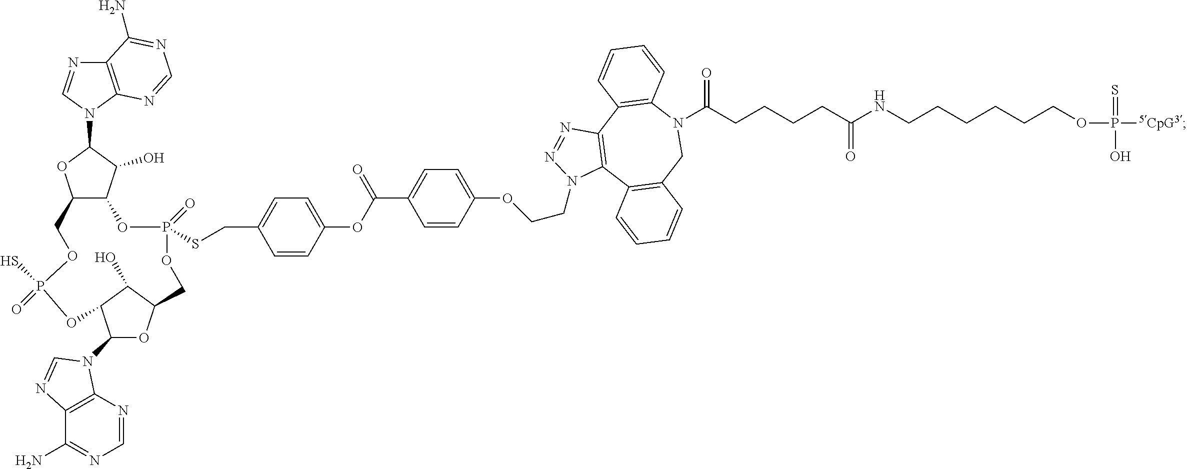

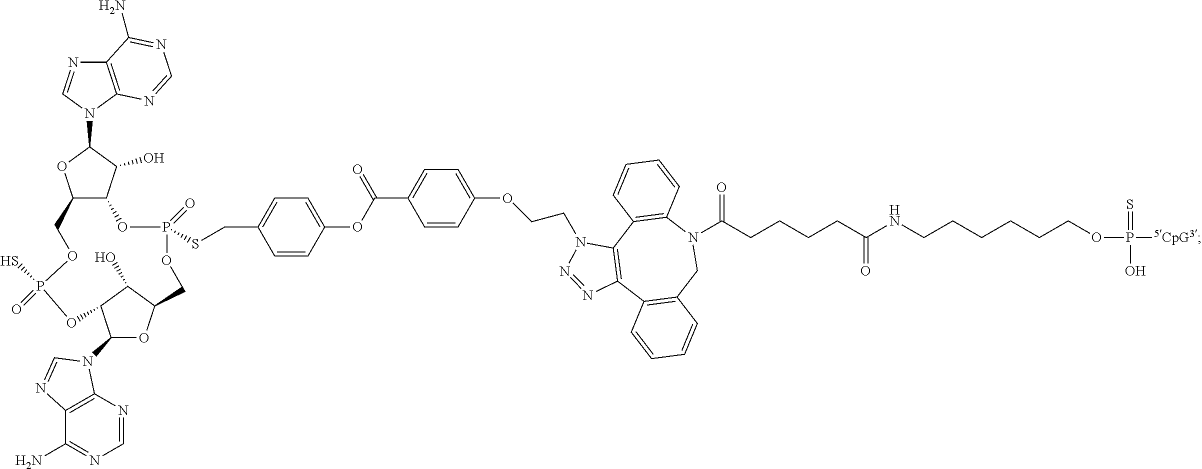

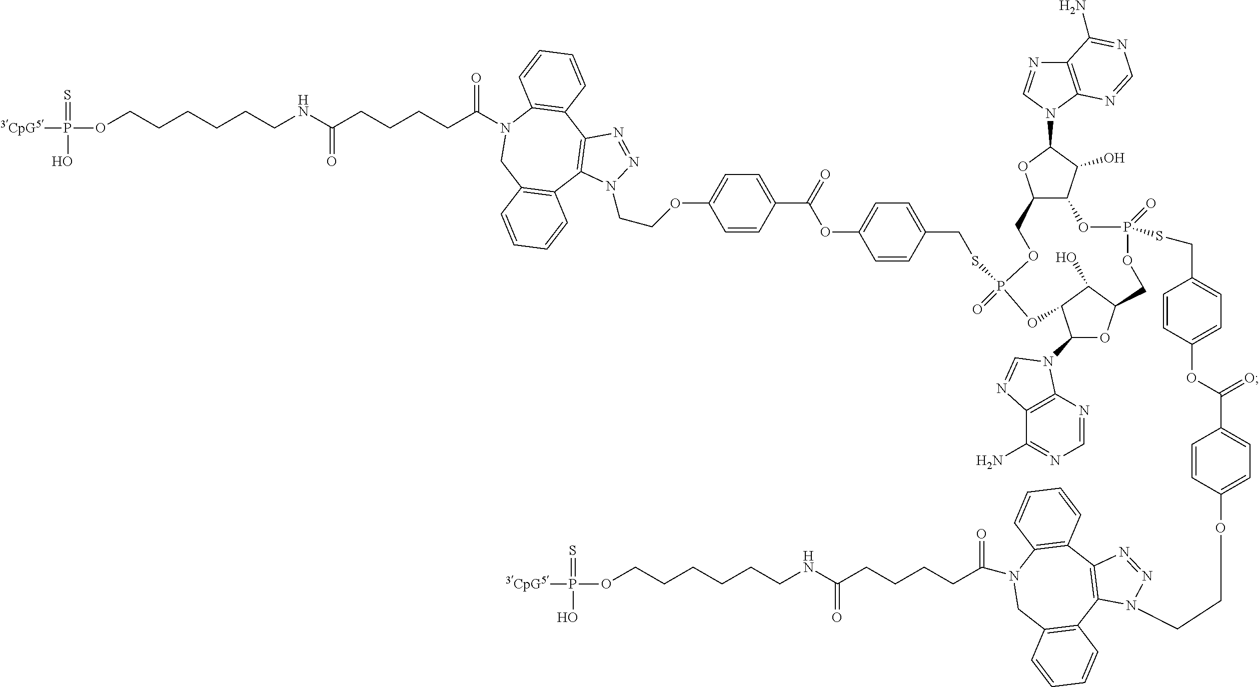

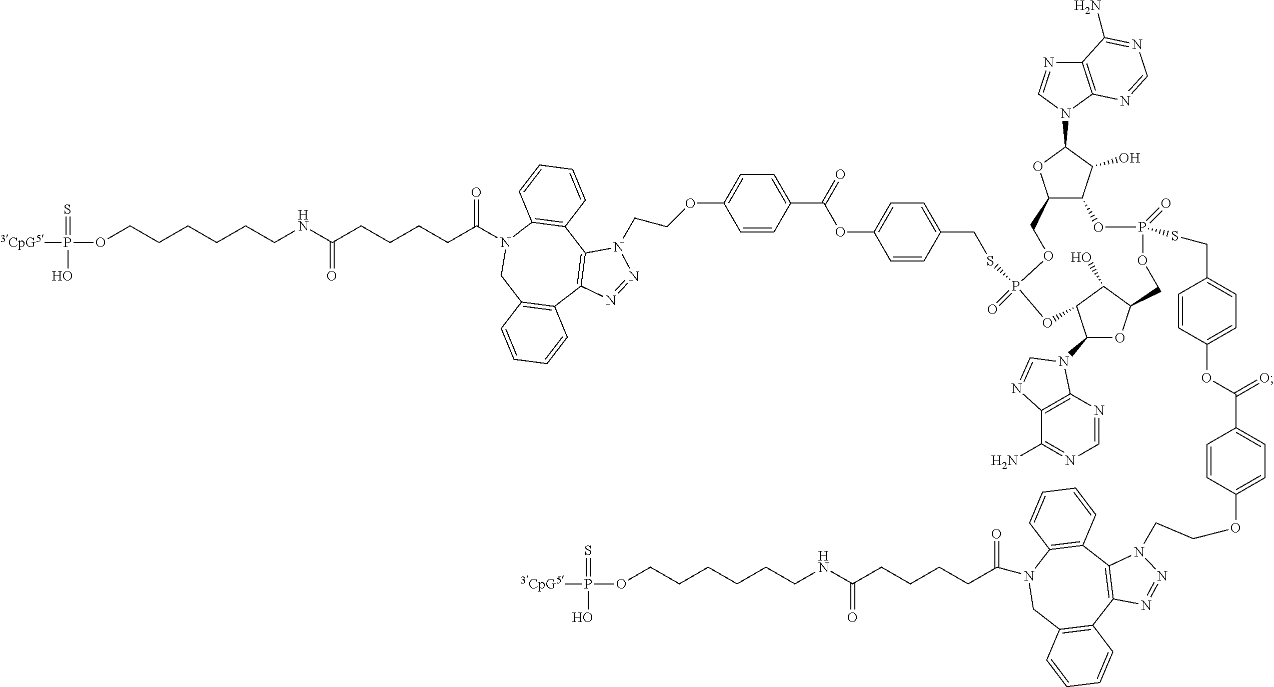

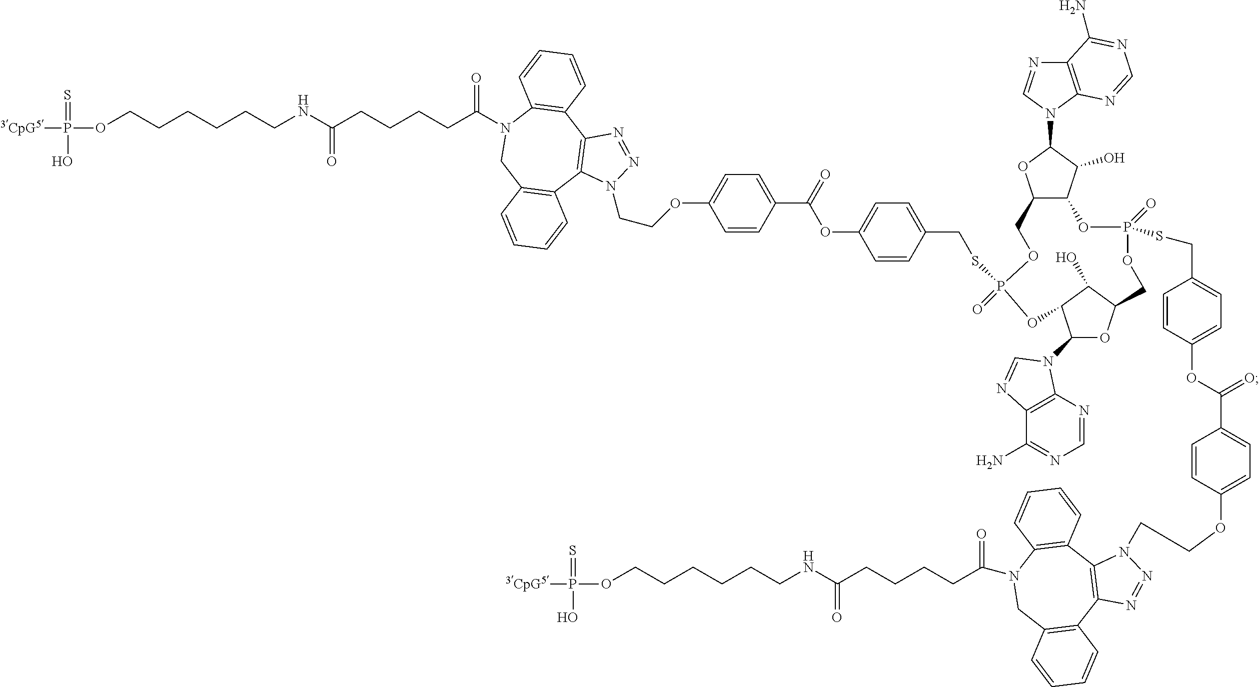

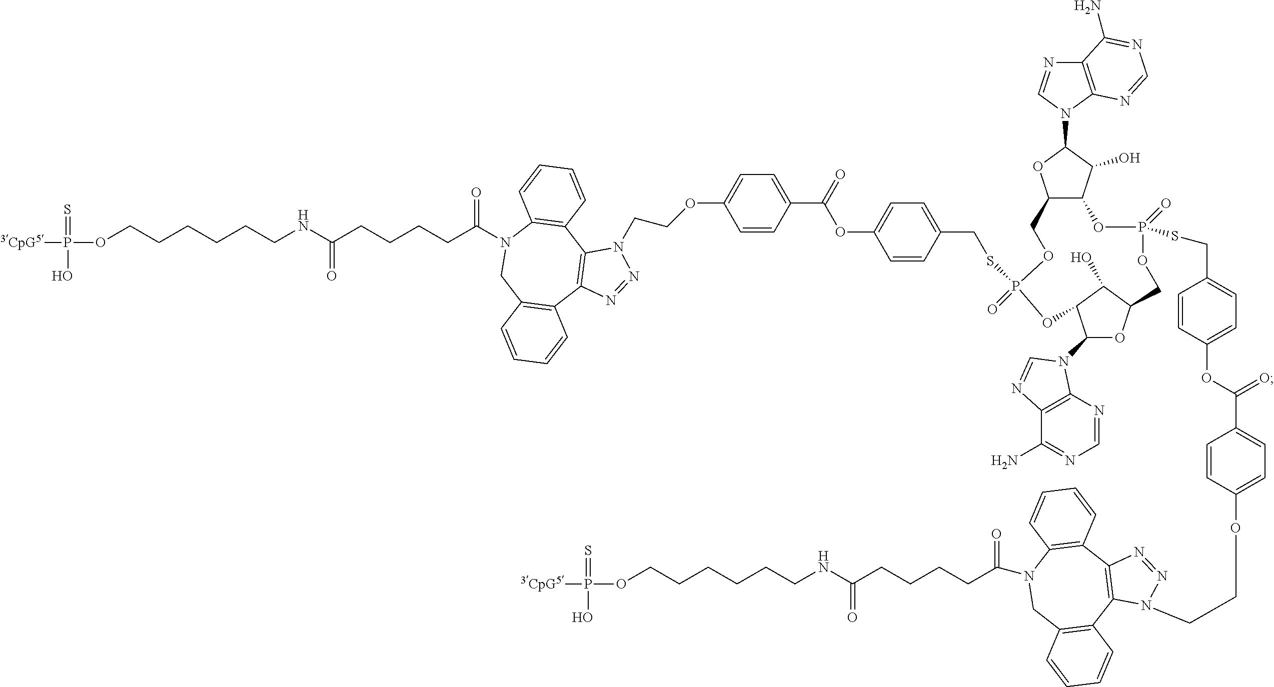

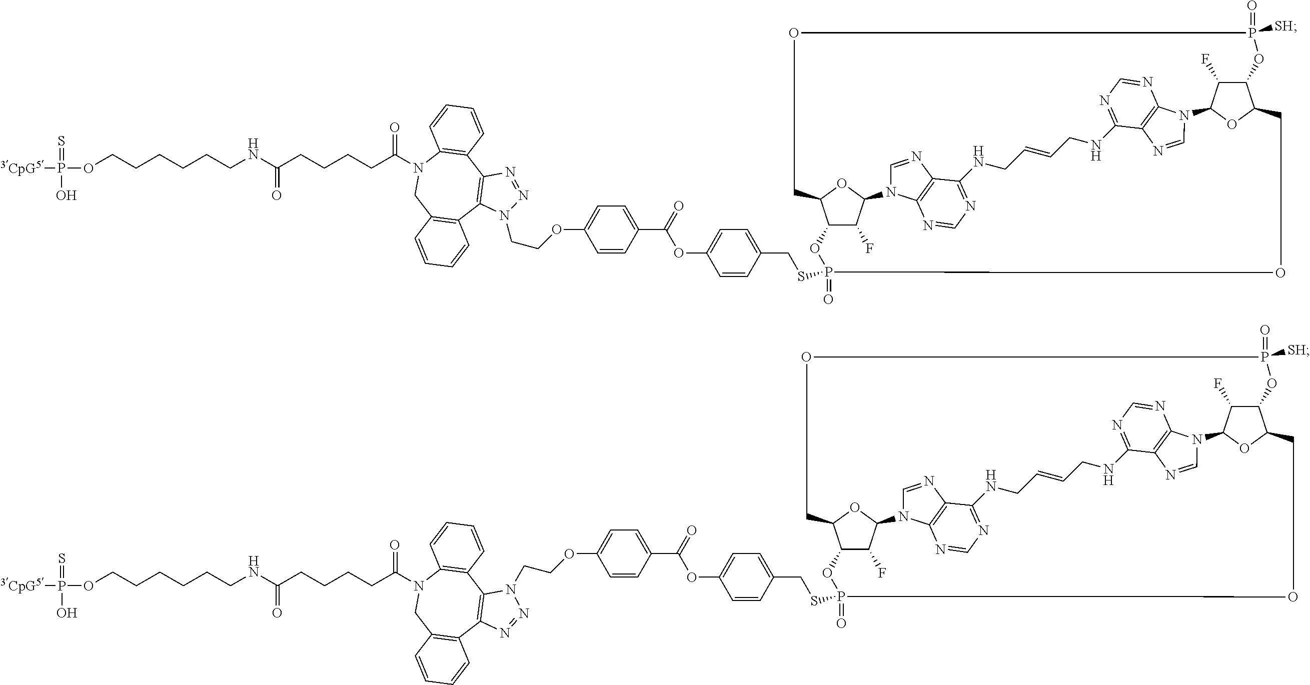

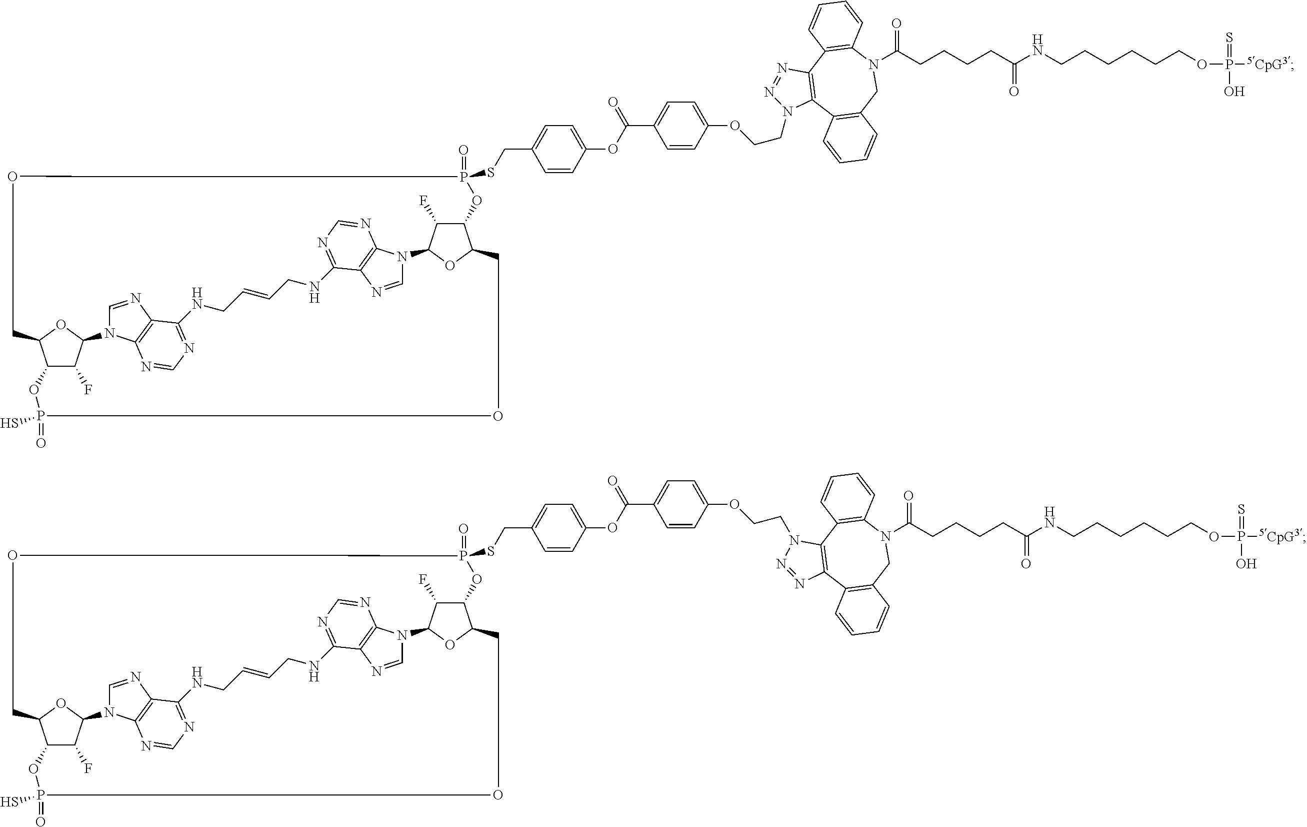

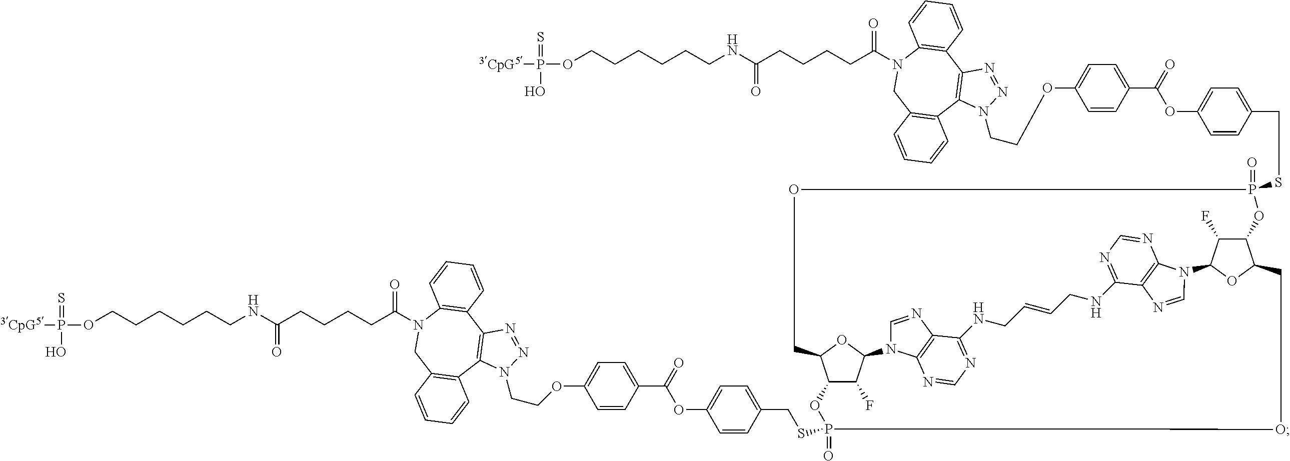

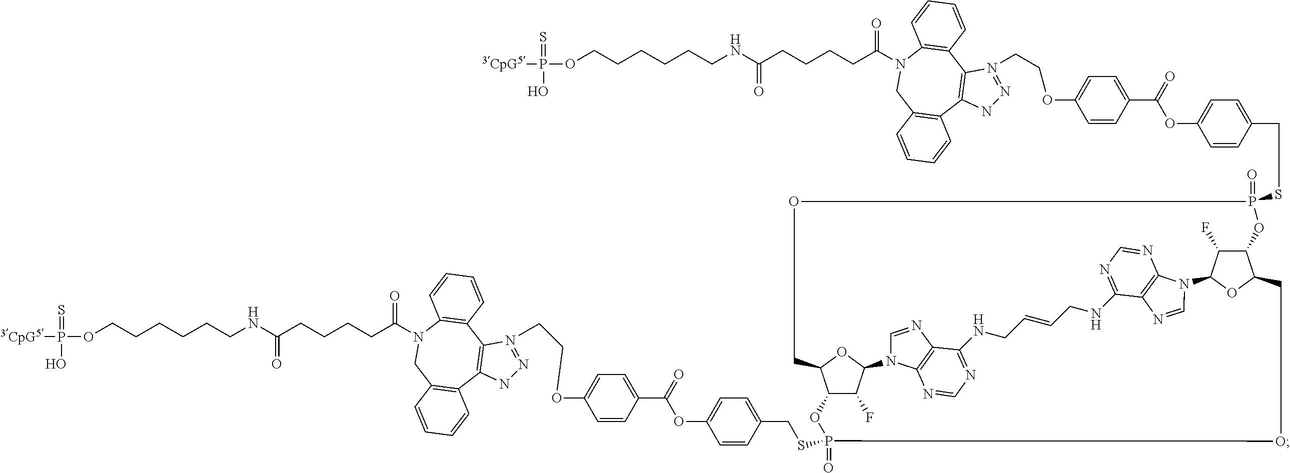

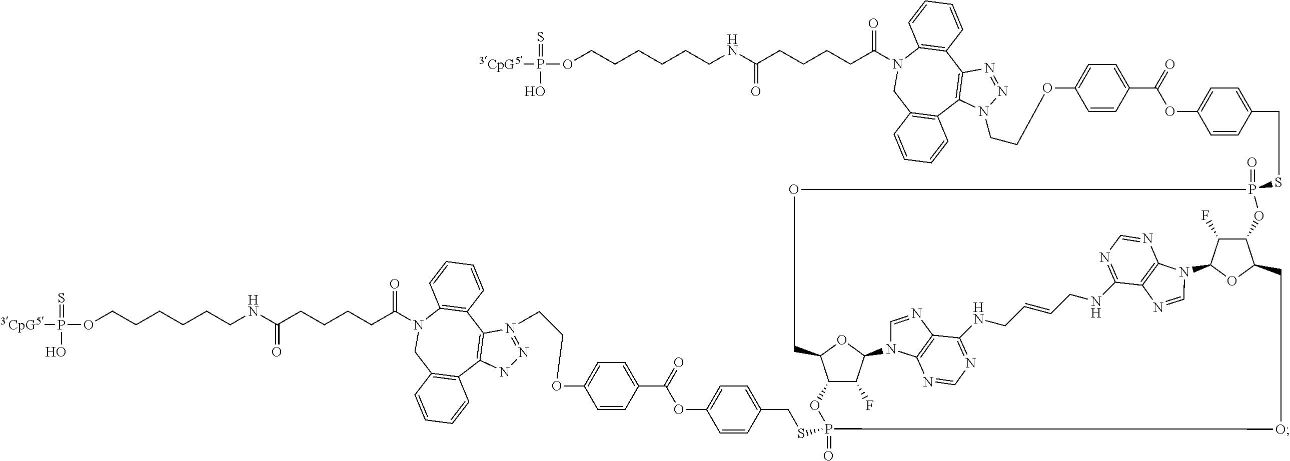

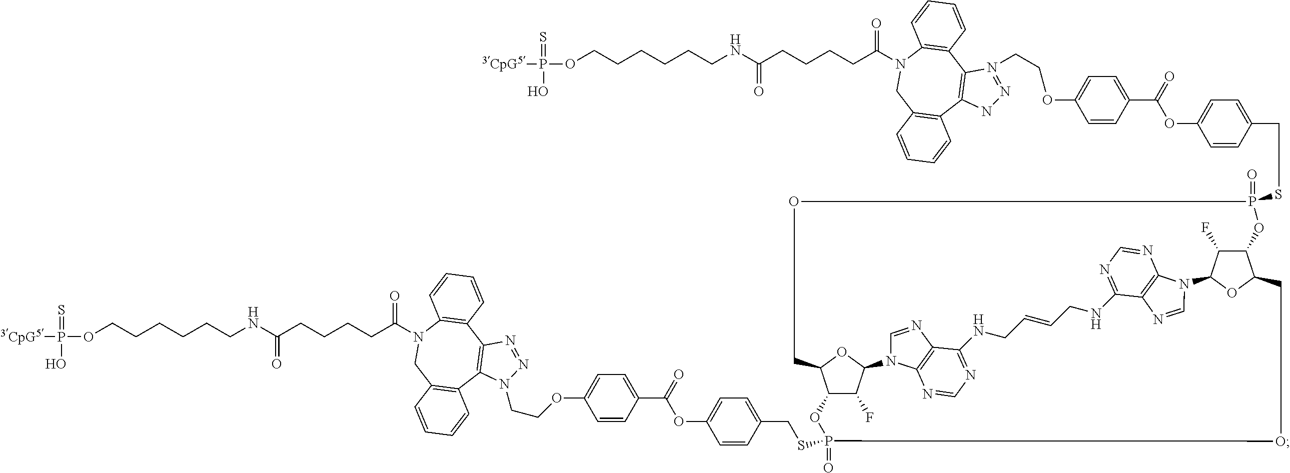

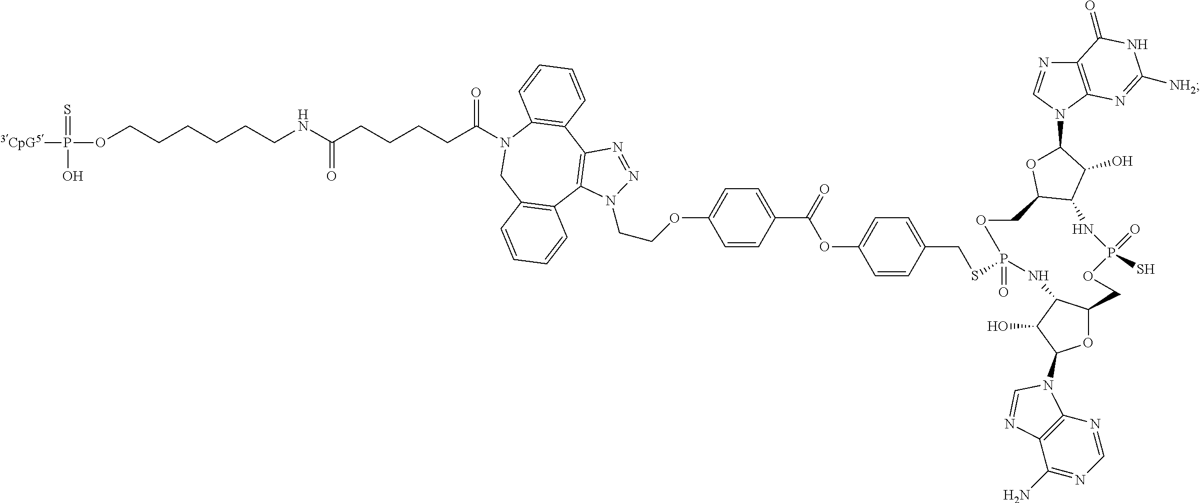

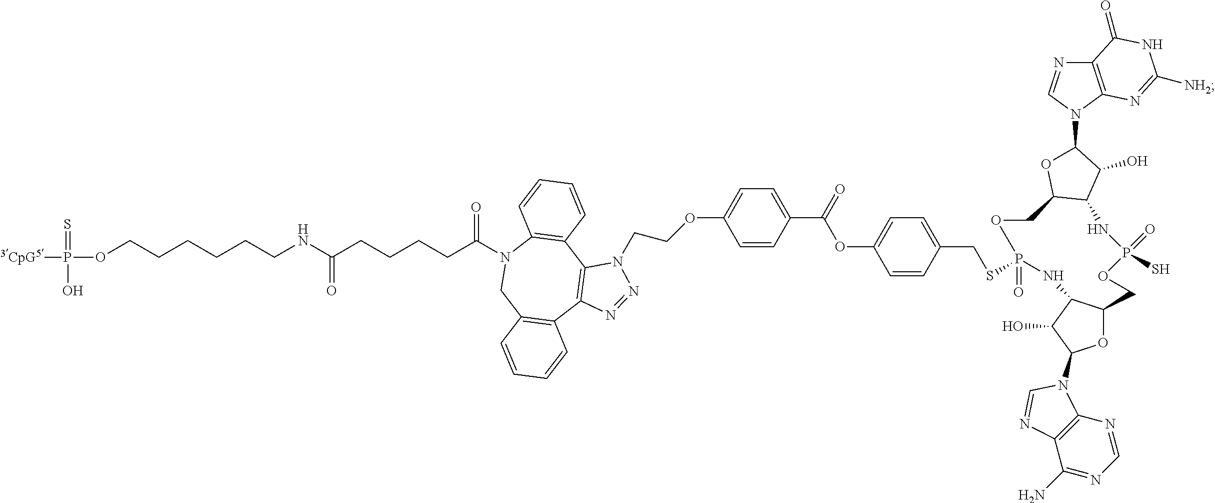

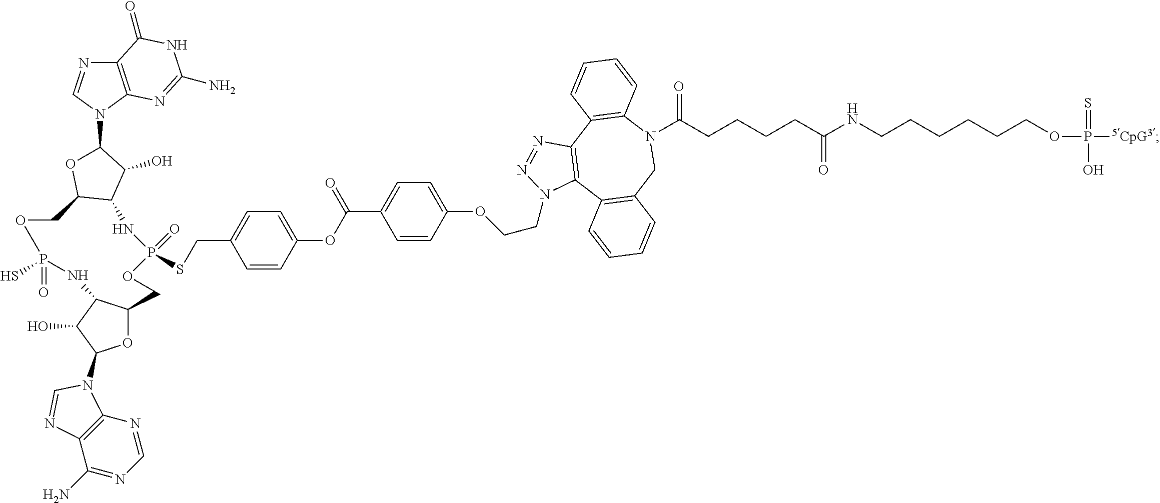

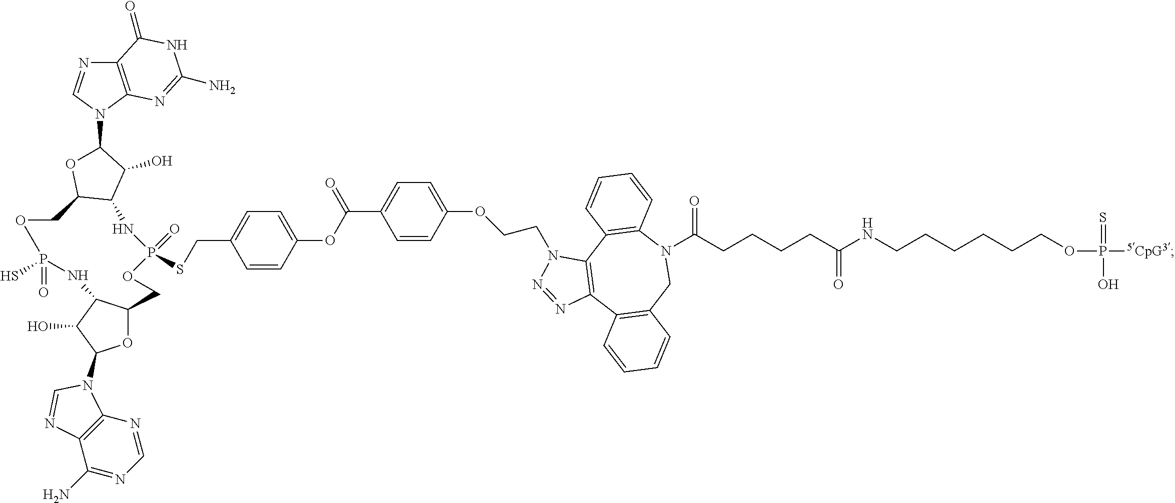

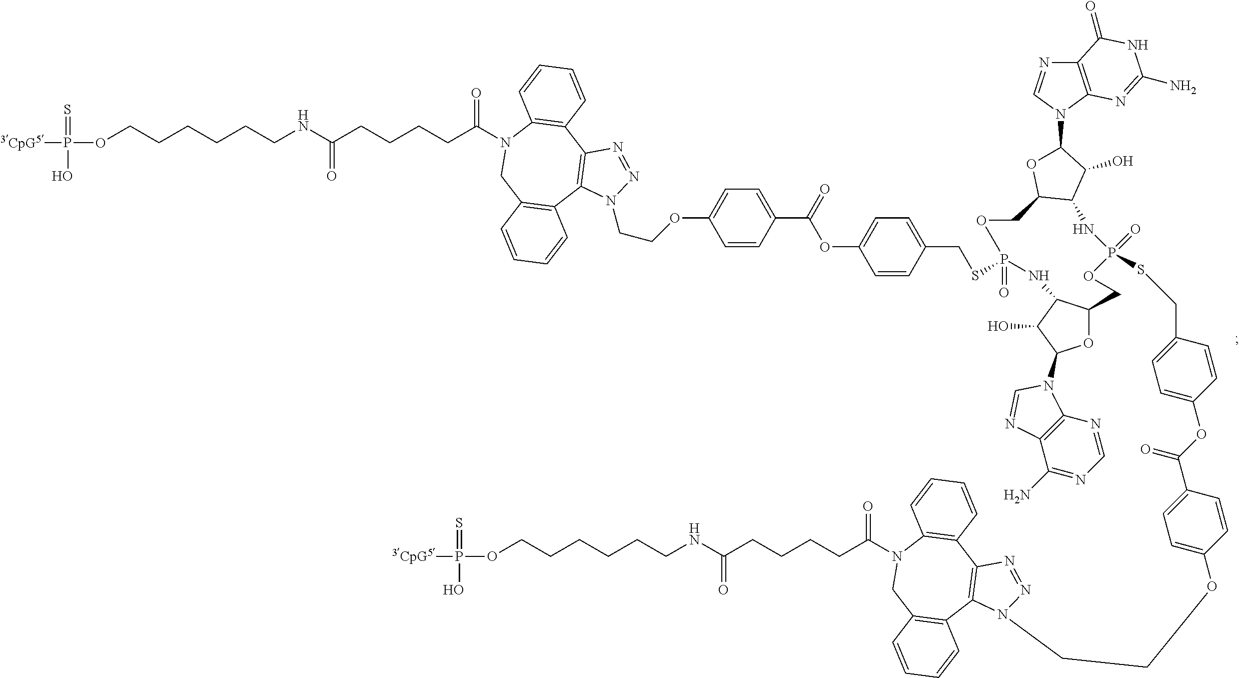

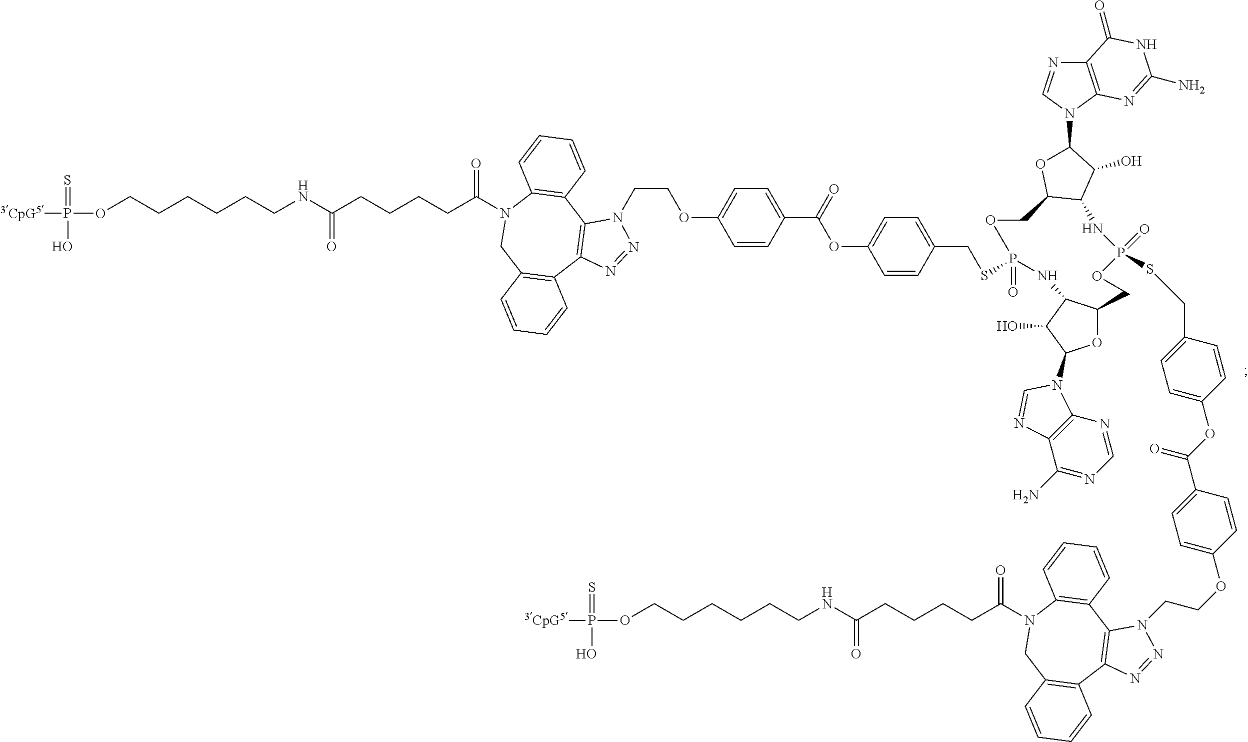

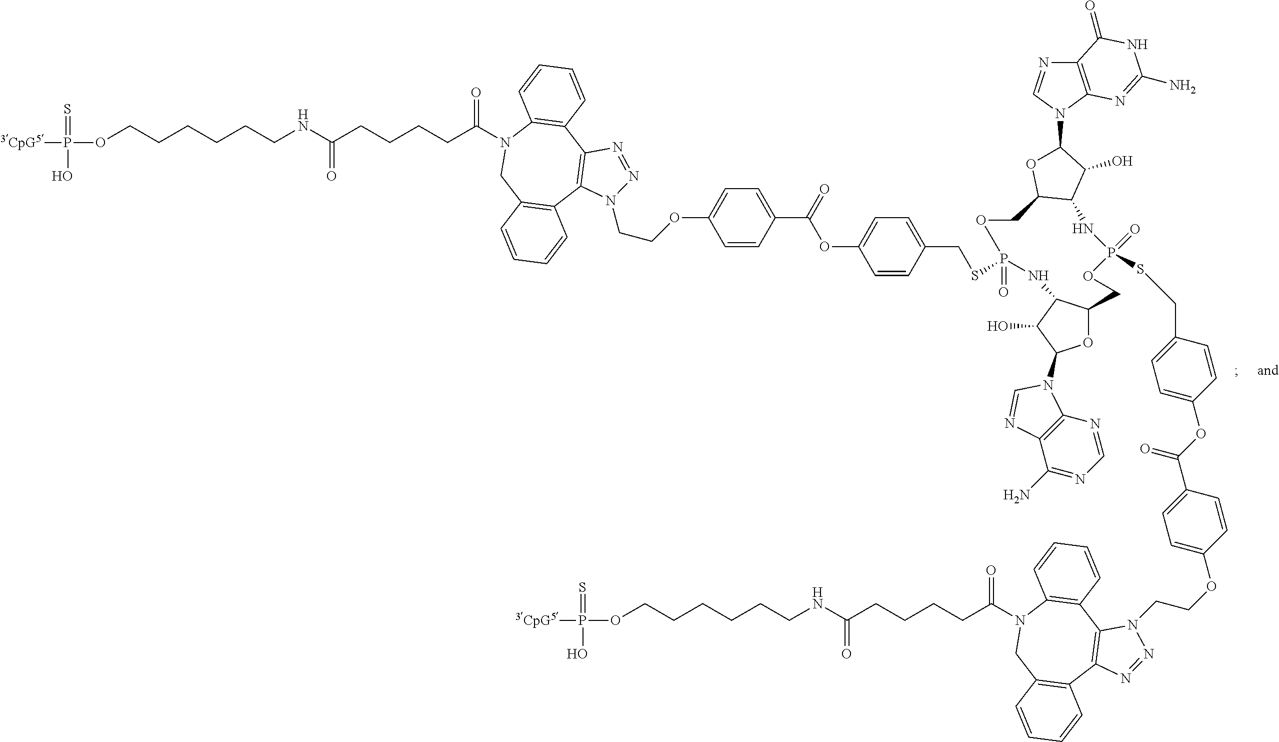

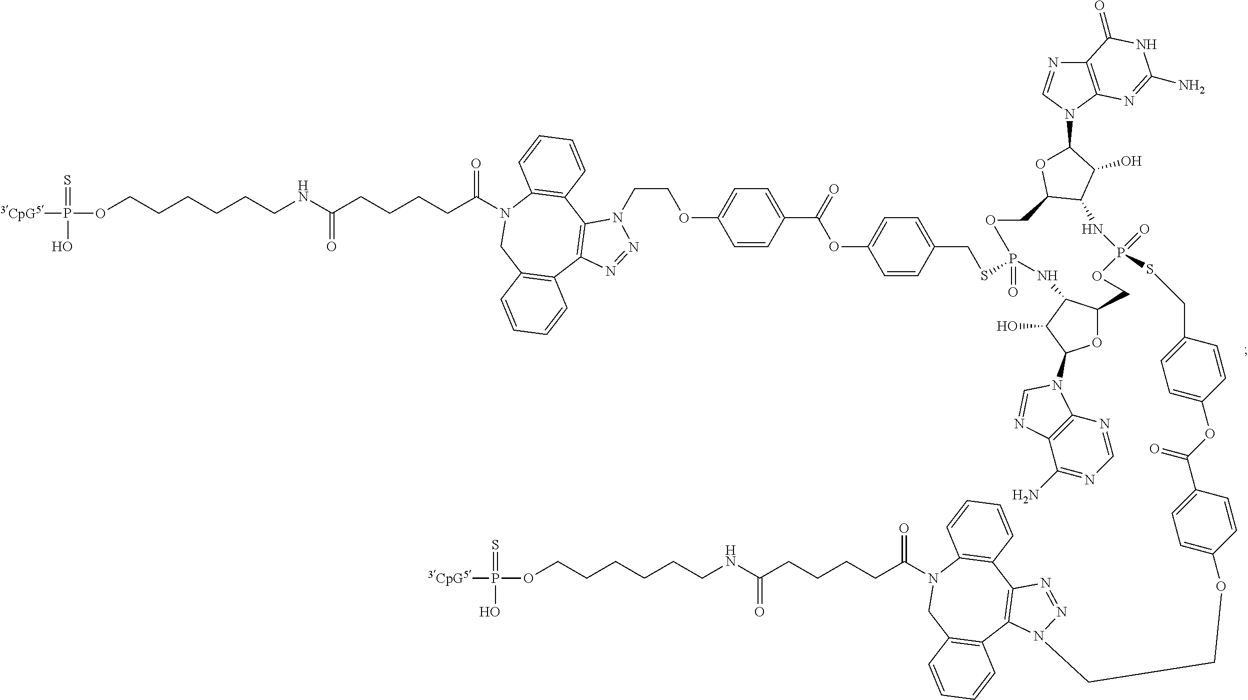

wherein CpG is a phosphorothioate linked 5′-T*C*G *A*A*C *G*T*T *C*G*A *A*C*G *T*T*C *G*A*A *C*G*T *T*C*G *A*A*T-3′ (SD-101); or a phosphorothioate linked 5′-T*C*G₁*A*A*C*G₁*T*T*C*G₁*-X-*G₁*C*T*T*G₁*C*A*A*G₁*C*T*-5′, wherein X is a glycerol linker and GI is 2′-deoxy-7-deazaguanosine (IMO-2125); or 5′-GGGGGGGGGGGACGATCGTCGGGGGGGGGG-3′ (CMP-001) or phosphorothioate linked 5′-T*C*G*T*C*G*T*T*T*T*G*T*C*G*T*T*T*T*G*T*C*G*T*T-3′ (PF-3512676), connecting at 3′-O or 5′-O of the terminal nucleotide.

In certain embodiments of the conjugate of formulae (XI), (XI-A), (XI-B), (XII), (XII-A), (XII-B), (XV), (XV-A), (XV-B), (XXIV), (XXV), (XXVI), (XXVI-A), (XXVI-B), (XXVII), (XXVII-A), and (XXVII-B), CpG is connected at 3′-O of the terminal nucleotide. In certain embodiments, CpG is connected at 5′-O of the terminal nucleotide.

In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide. In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101), connecting at 5′-O of the terminal nucleotide.

In certain embodiments of the conjugate of formulae (XI), (XI-A), (XI-B), (XII), (XII-A), (XII-B), (XV), (XV-A), (XV-B), (XXIV), (XXV), (XXVI), (XXVI-A), (XXVI-B), (XXVII), (XXVII-A), and (XXVII-B), b is one.

In certain embodiments of the conjugate of formulae (XI), (XI-A), (XI-B), (XII), (XII-A), (XII-B), (XV), (XV-A), (XV-B), (XXIV), (XXV), (XXVI), (XXVI-A), (XXVI-B), (XXVII), (XXVII-A), and (XXVII-B), b is two.

In certain embodiments of the conjugate of formulae (XI), (XI-A), (XI-B), (XII), (XII-A), (XII-B), (XIII), (XIII-A), (XIII-B), (XIV), (XIV-A), (XIV-B), (XV), (XV-A), (XV-B), (XXVI), (XXVI-A), (XXVI-B), (XXVII), (XXVII-A), and (XXVII-B), wherein b is one and the cyclic dinucleotide is selected from:

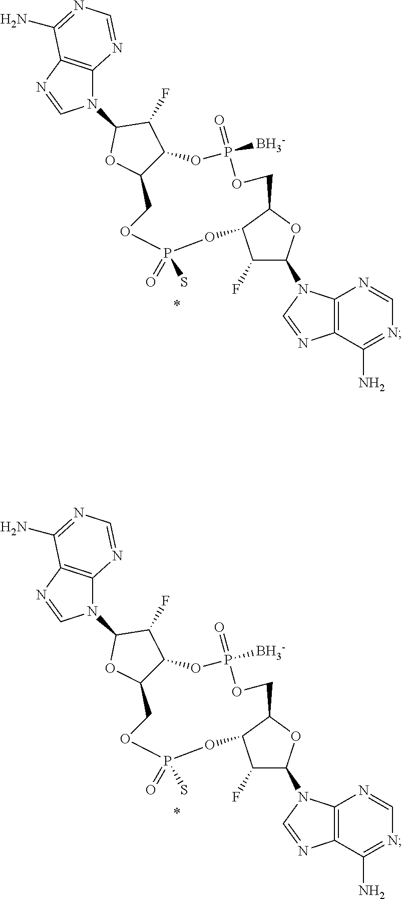

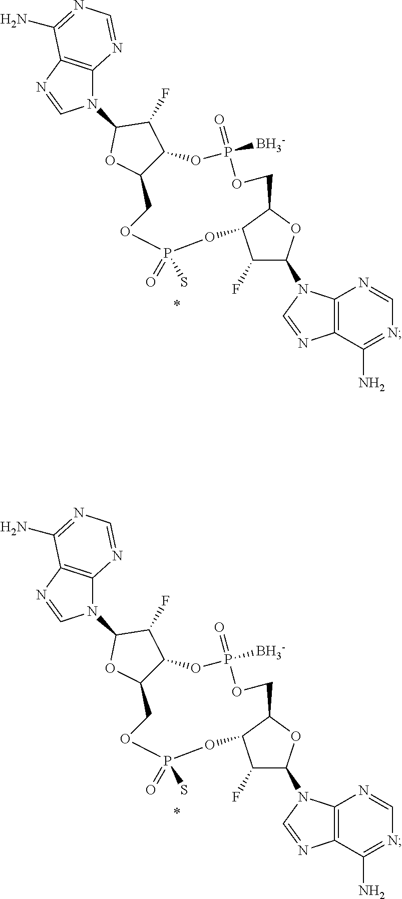

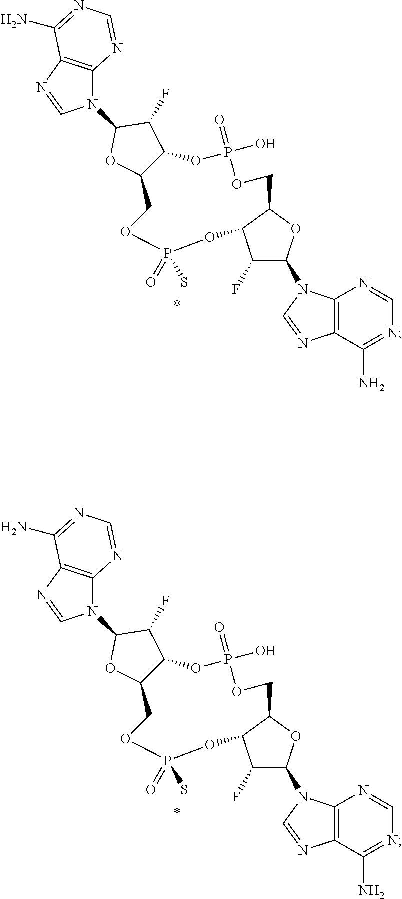 [SMILES: Nc1ncnc2c1ncn2[C@@H]1O[C@@H]2CO[P@@](=O)(S)O[C@H]3[C@@H](F)[C@H](n4cnc5c(N)ncnc54)O[C@@H]3COP(=O)(O)O[C@H]2[C@H]1F ; Nc1ncnc2c1ncn2[C@@H]1O[C@@H]2CO[P@](=O)(S)O[C@H]3[C@@H](F)[C@H](n4cnc5c(N)ncnc54)O[C@@H]3COP(=O)(O)O[C@H]2[C@H]1F]

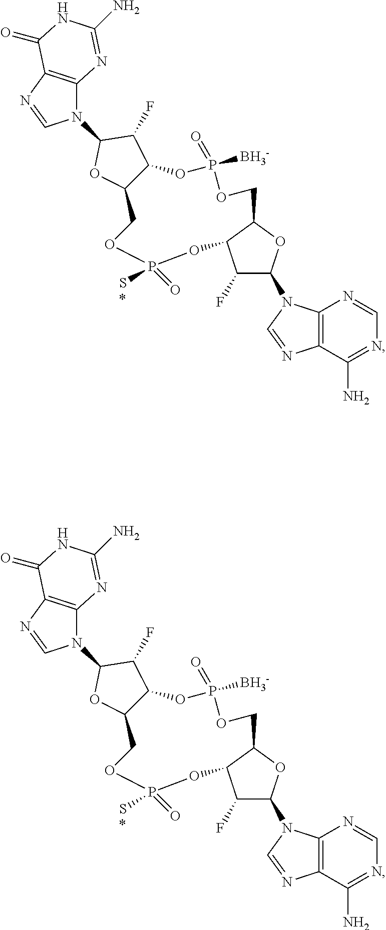

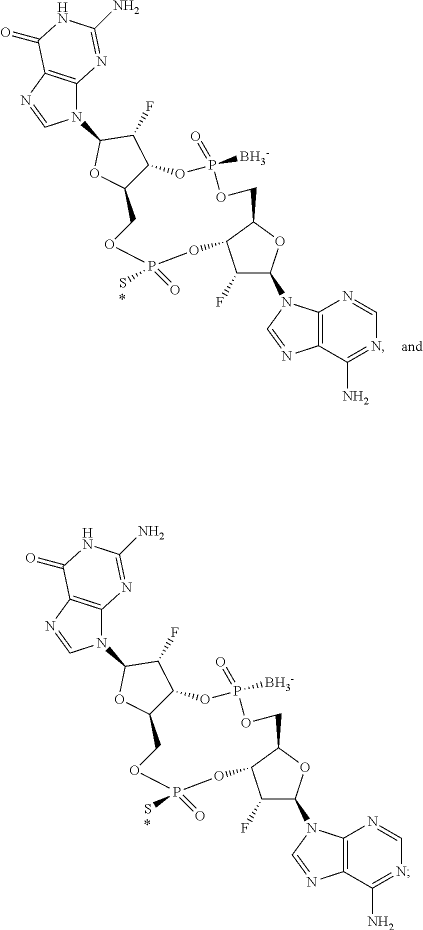

wherein the * indicates the S of thiol group (—SH, H not shown where * appears) of the cyclic dinucleotide that is connecting with the linker.

In certain embodiments, the conjugate has following structures:

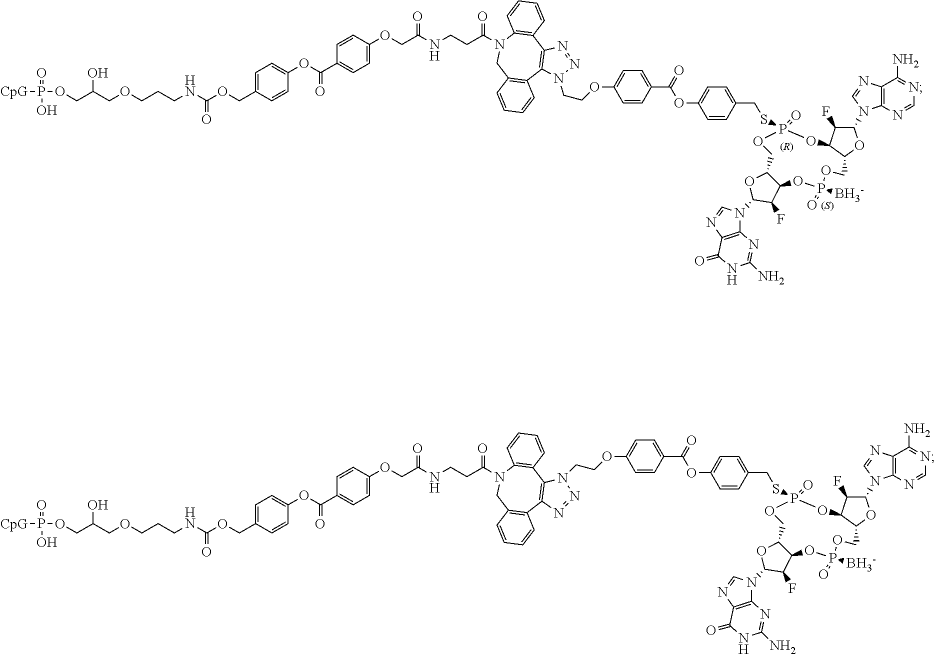

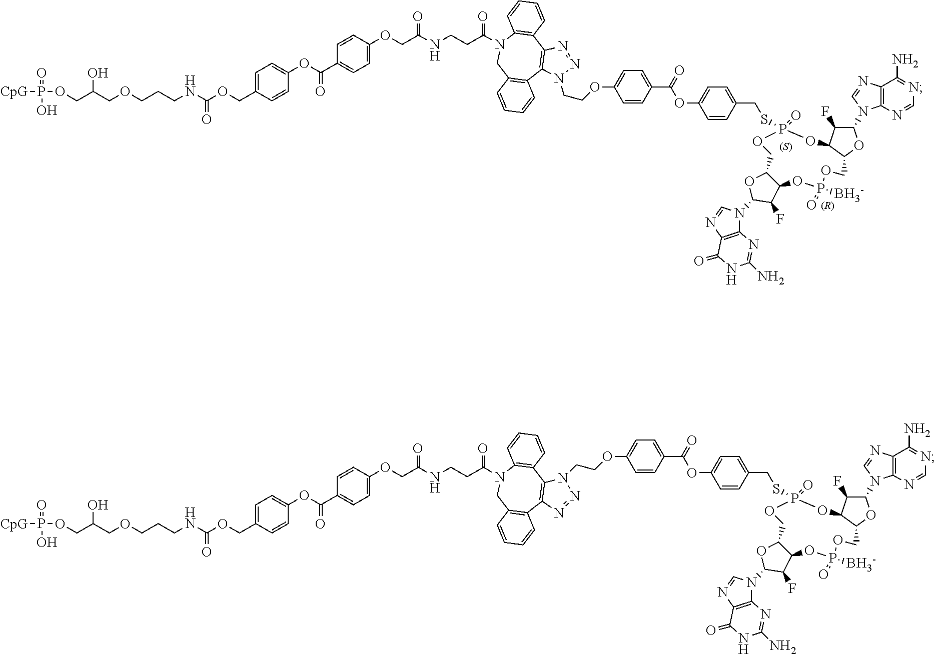

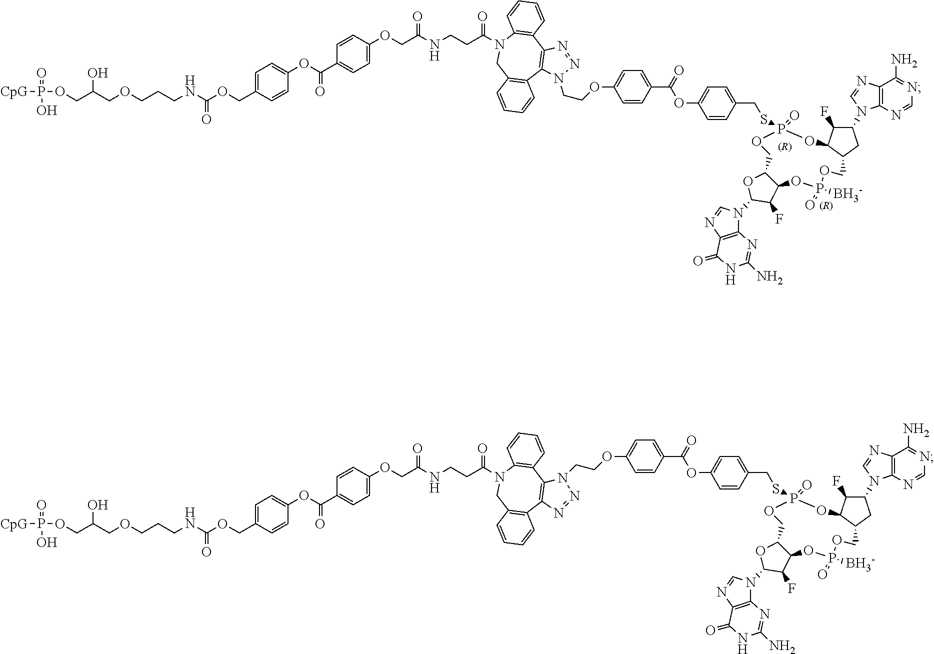

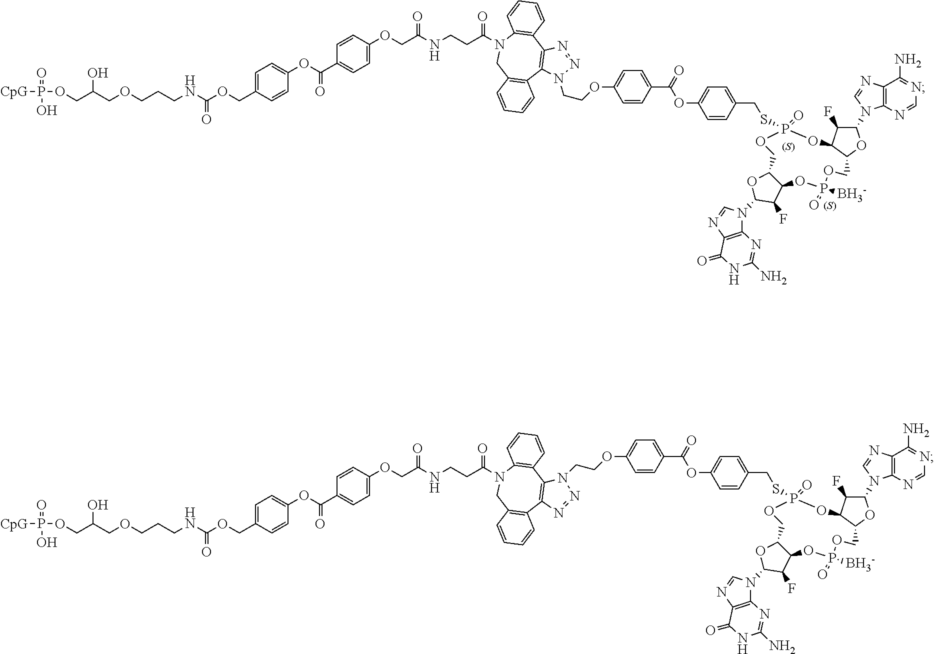

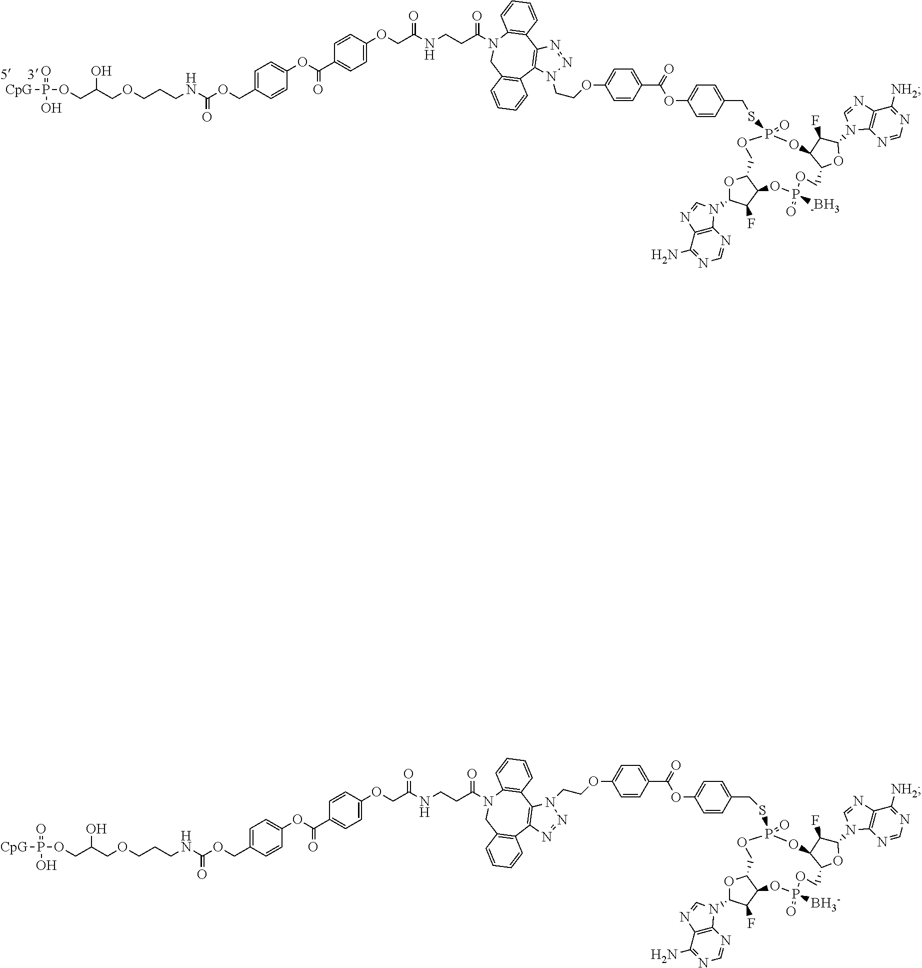

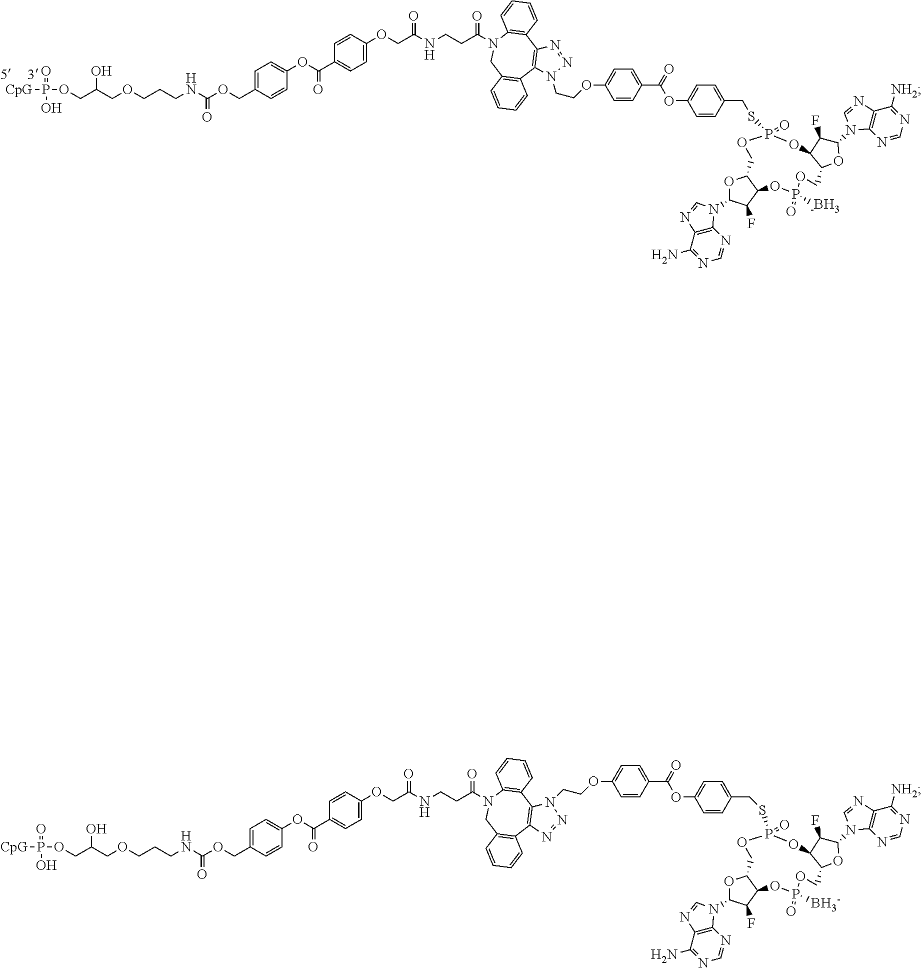

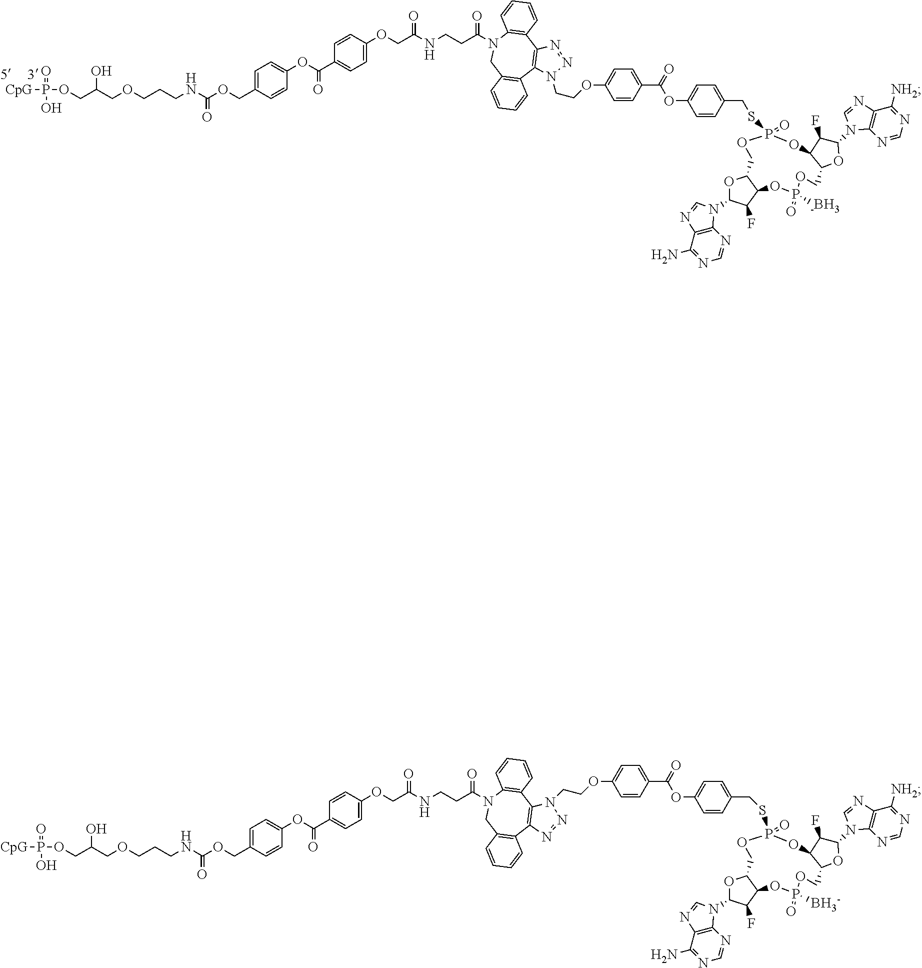

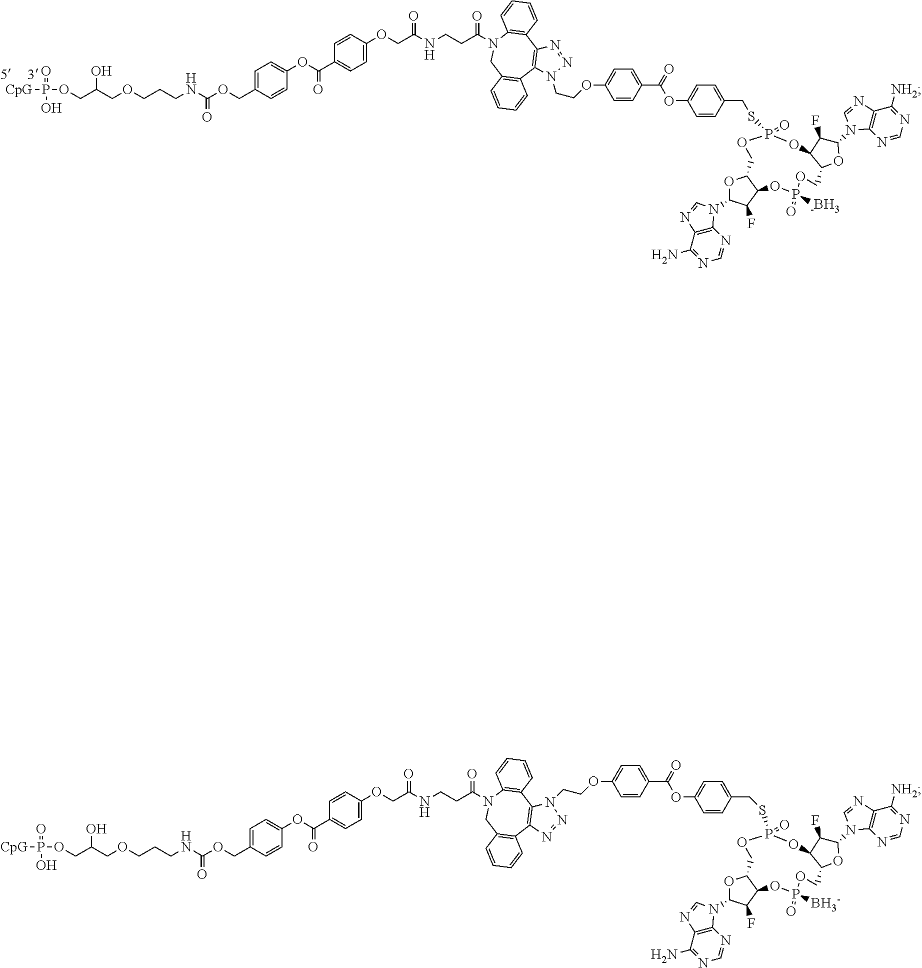

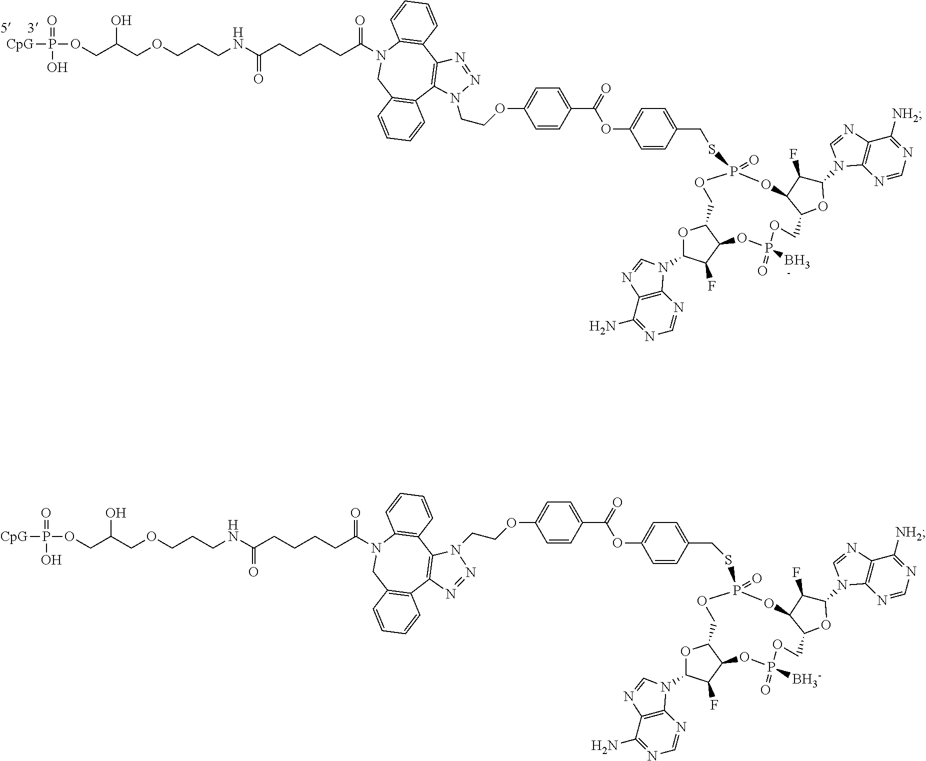

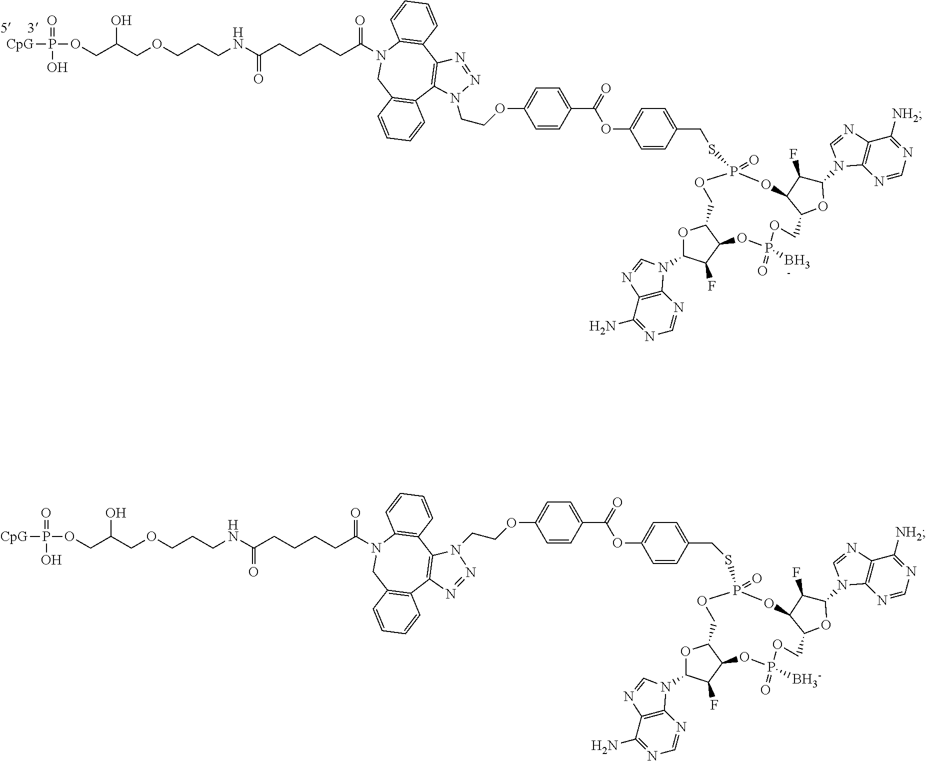

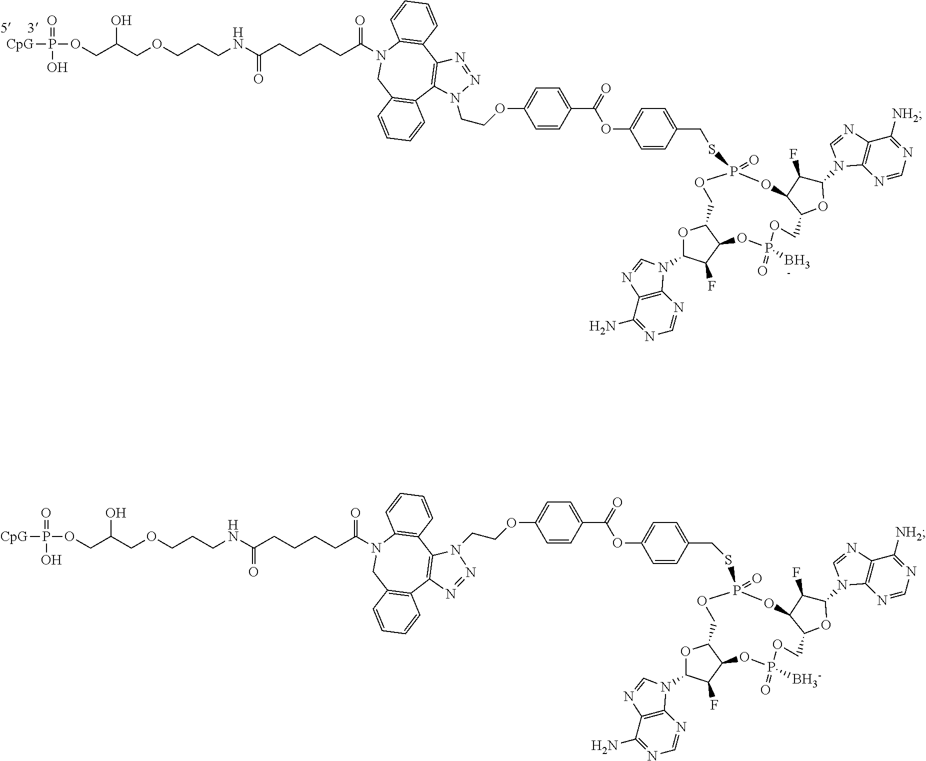

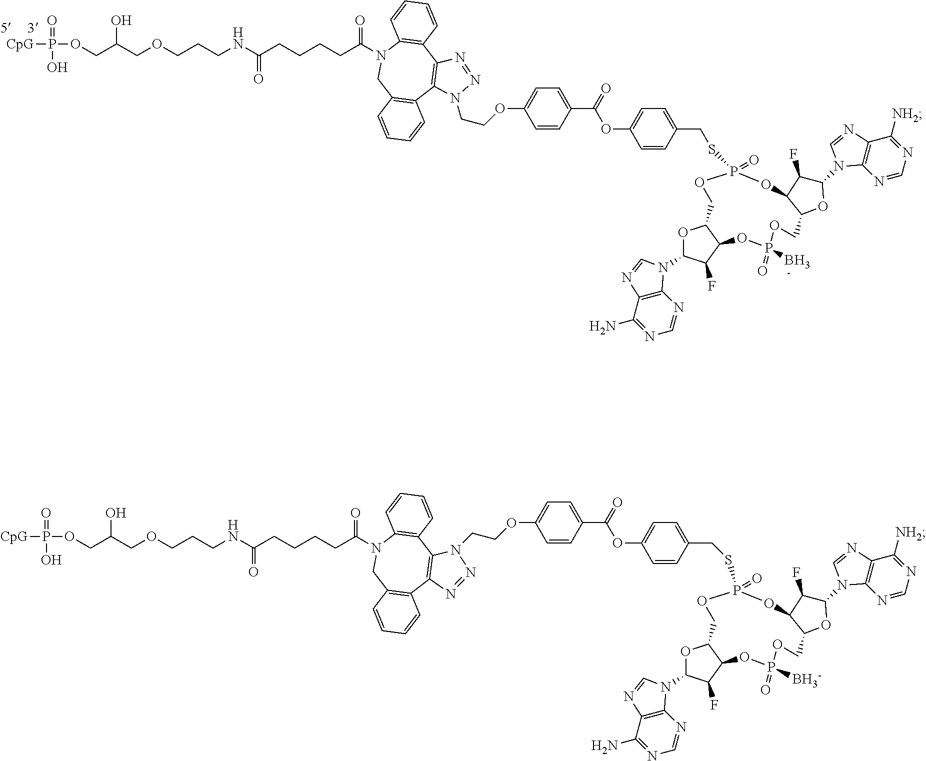

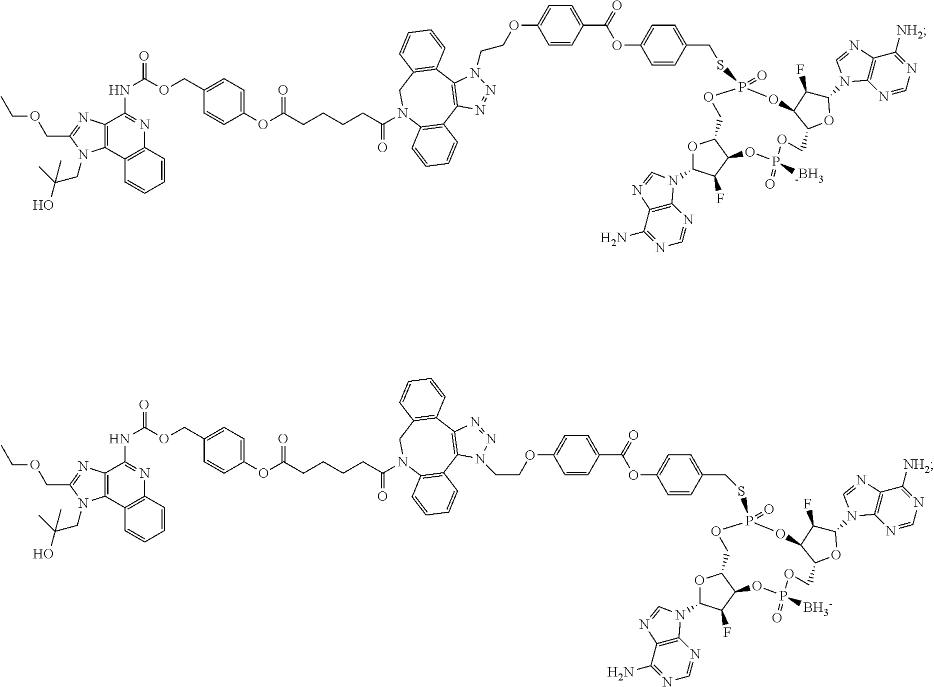

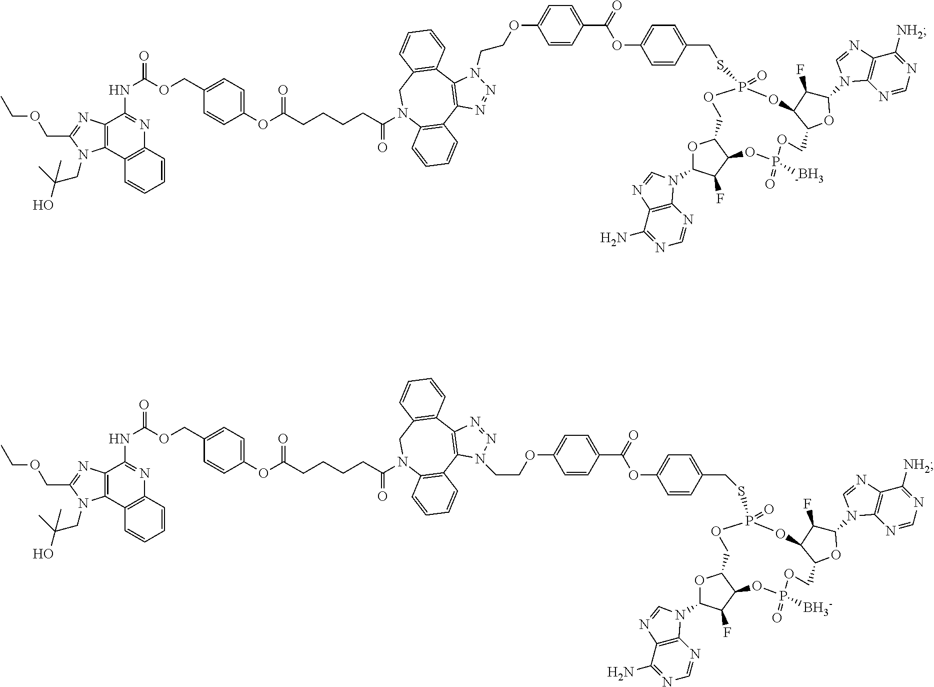

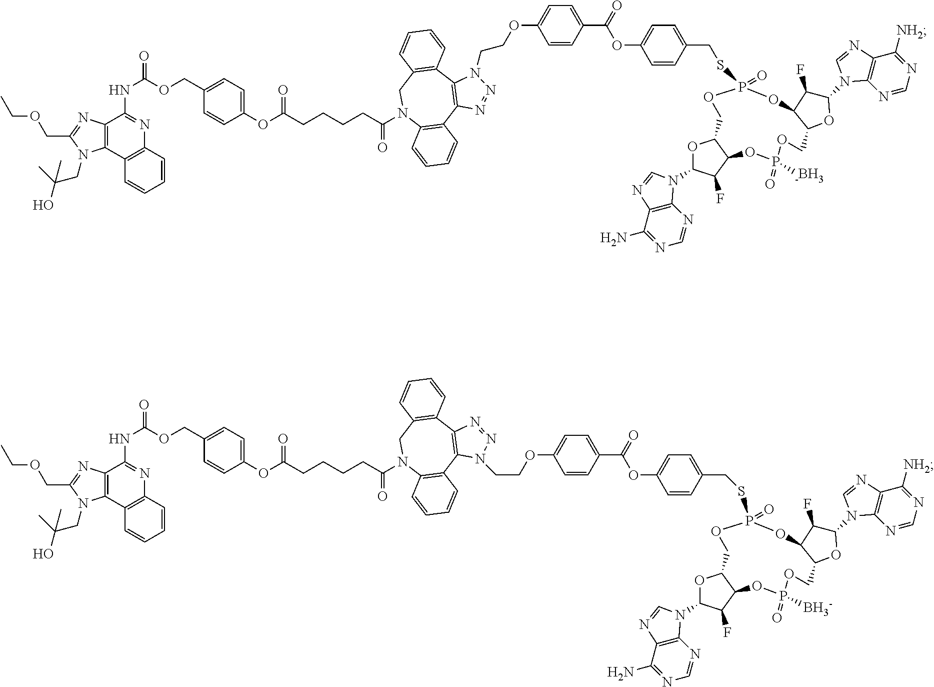

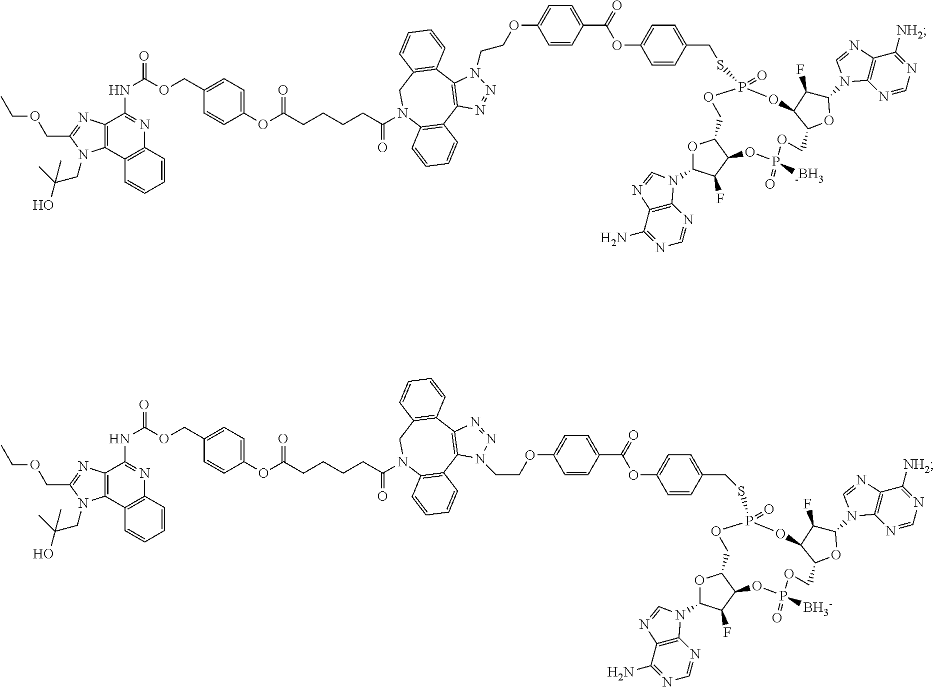

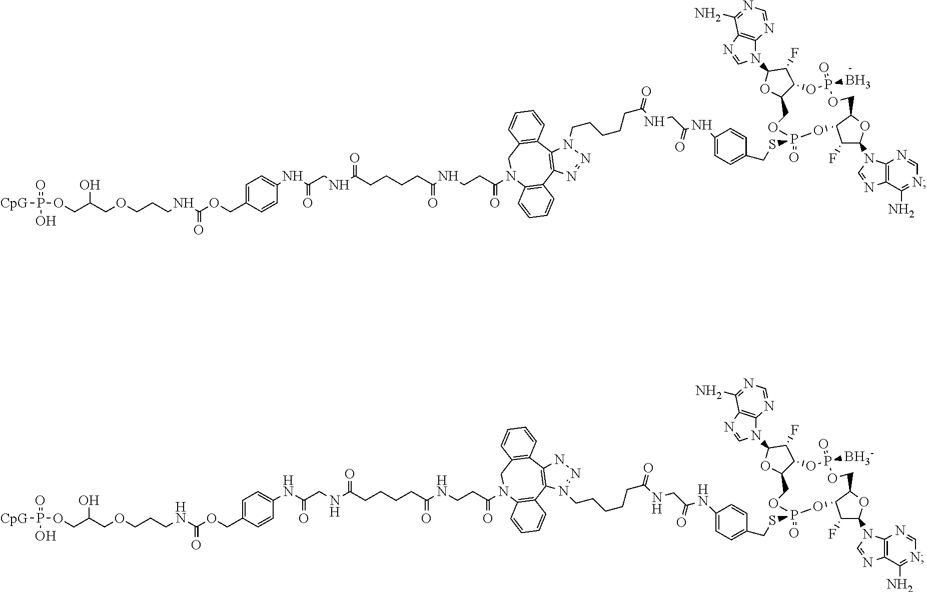

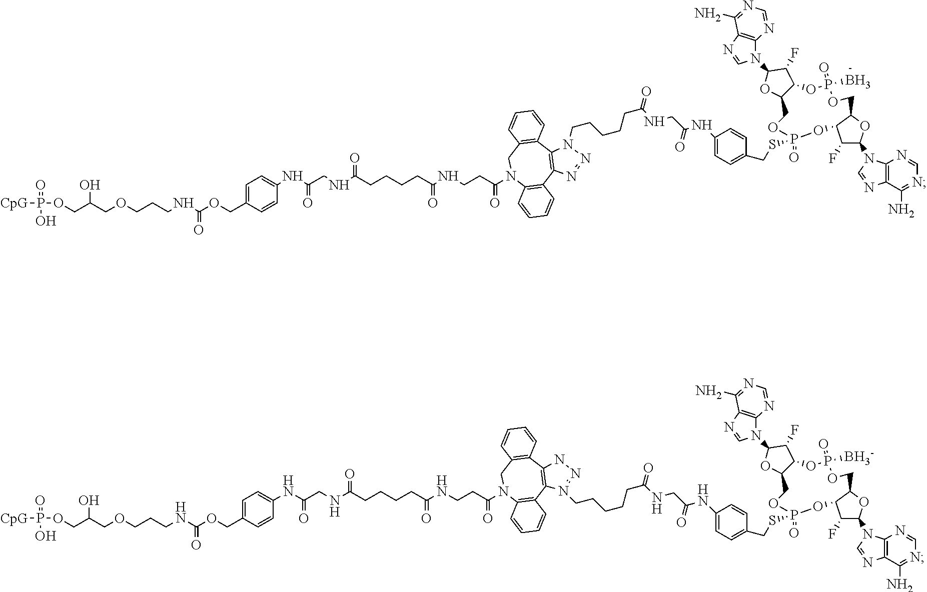

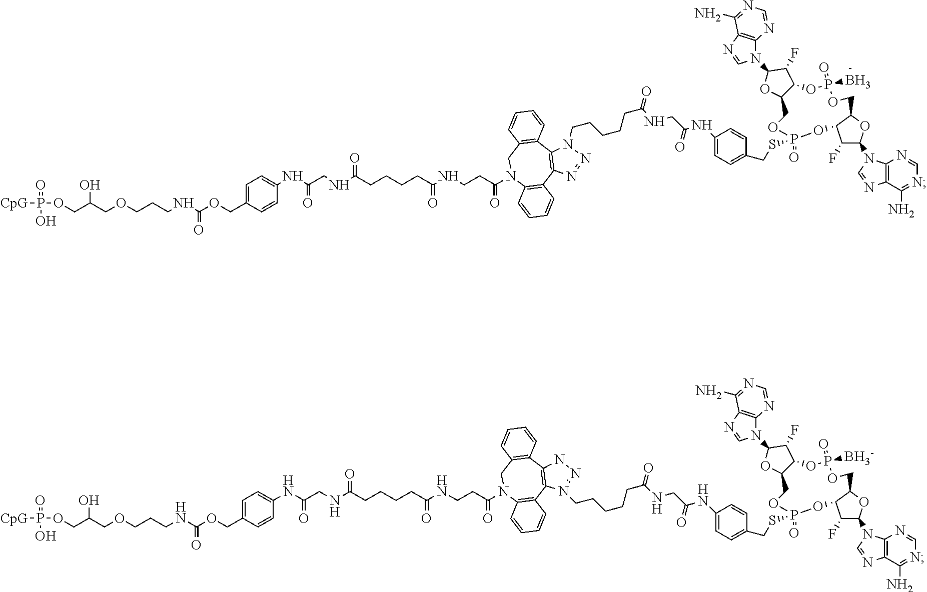

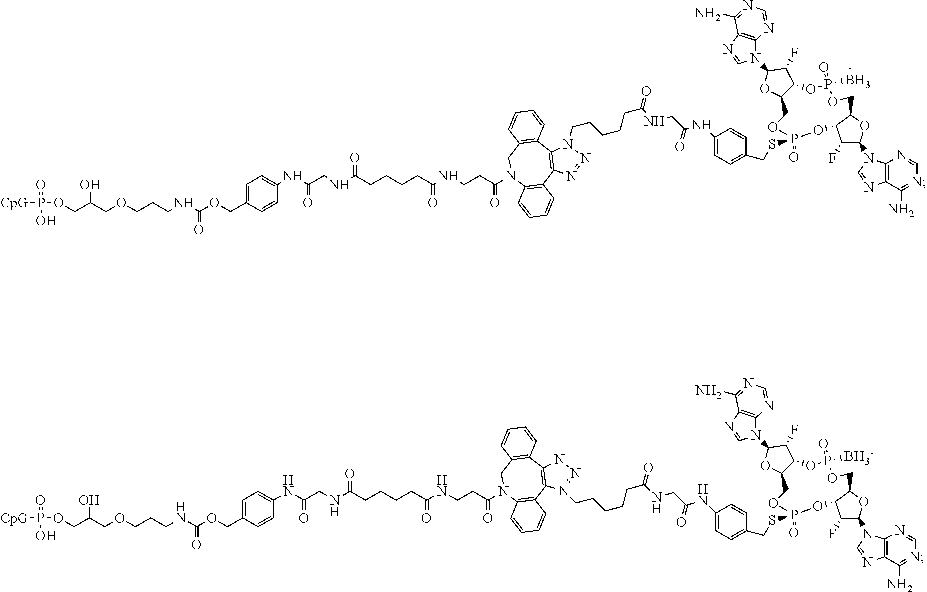

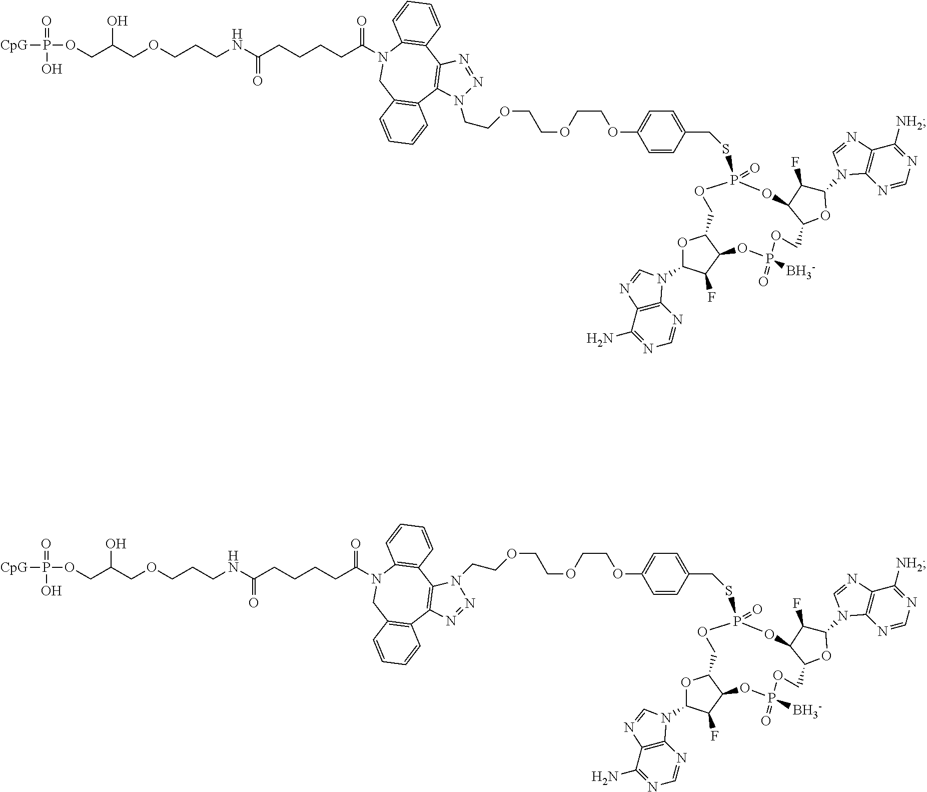

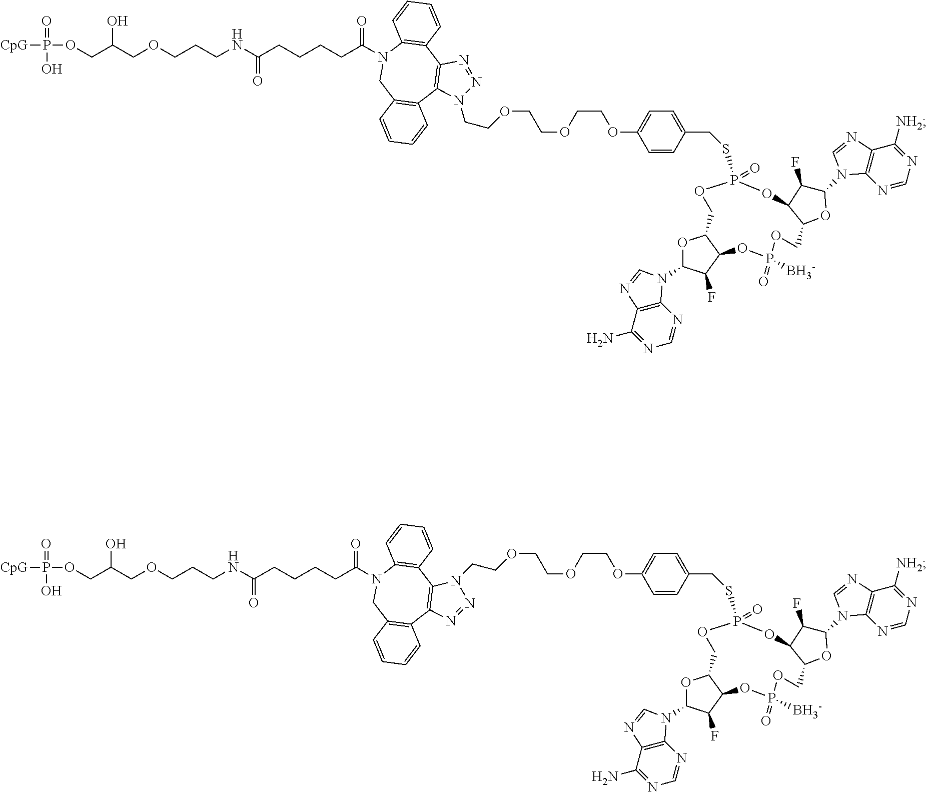

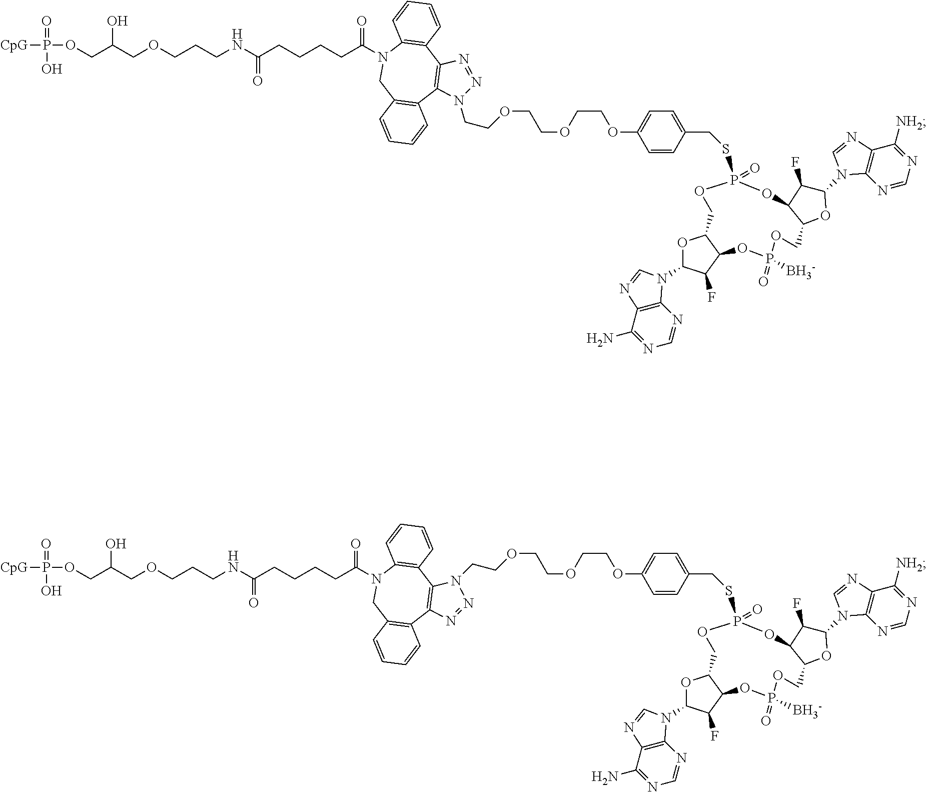

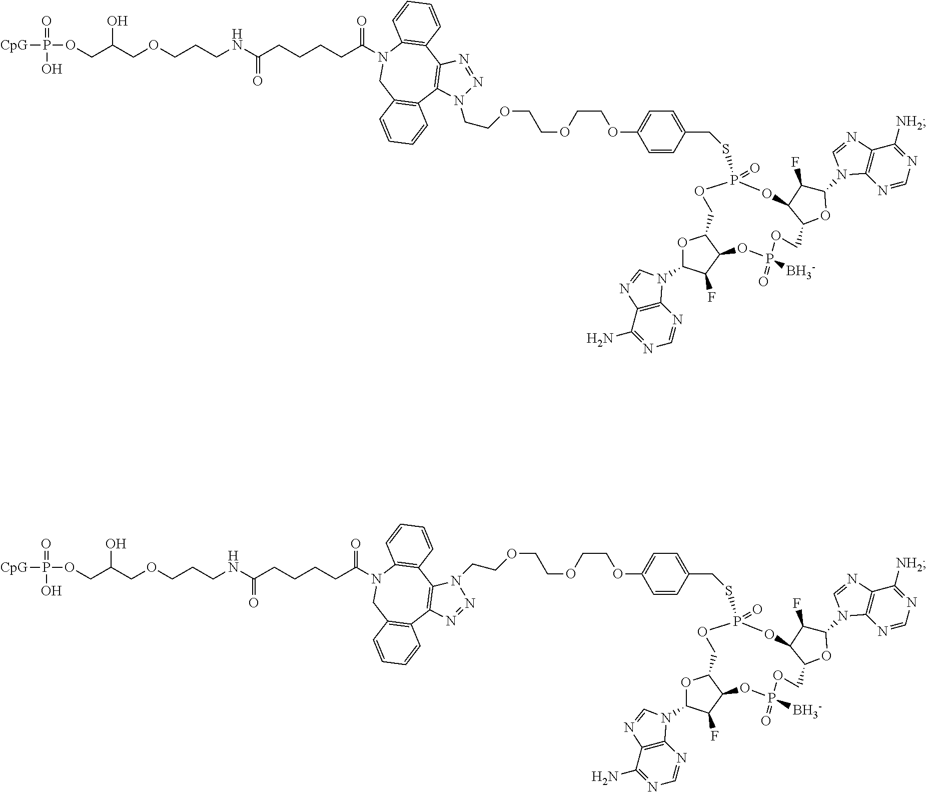

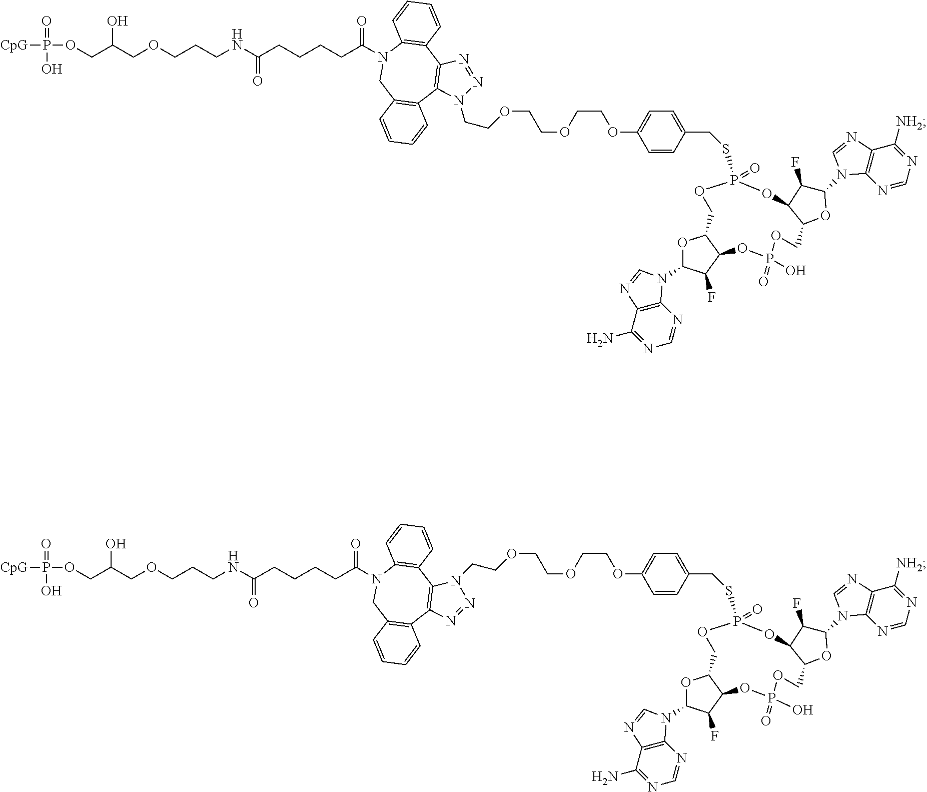

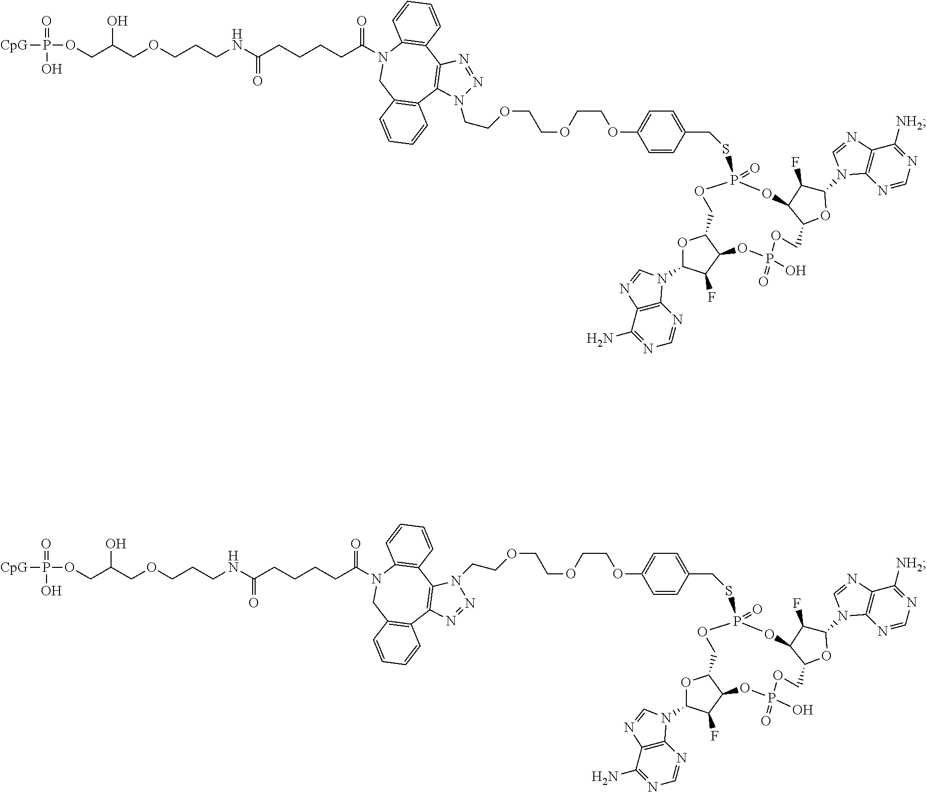

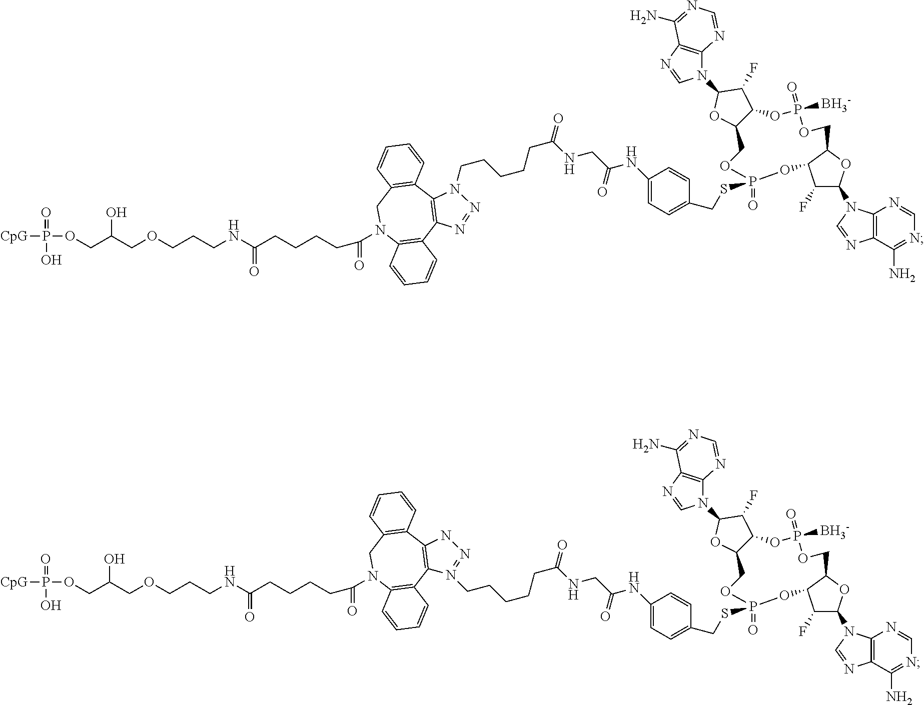

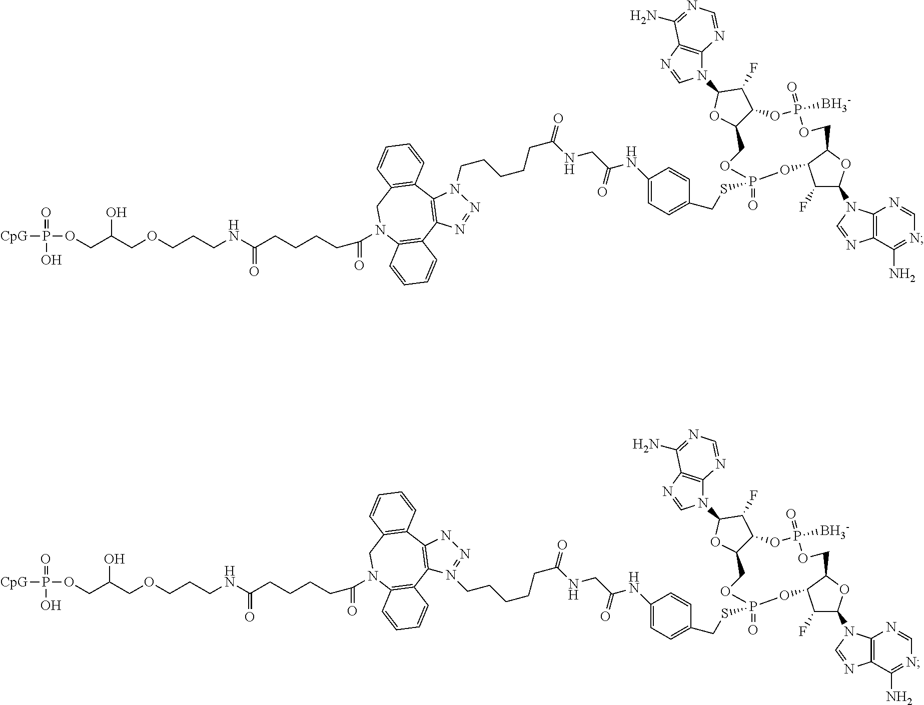

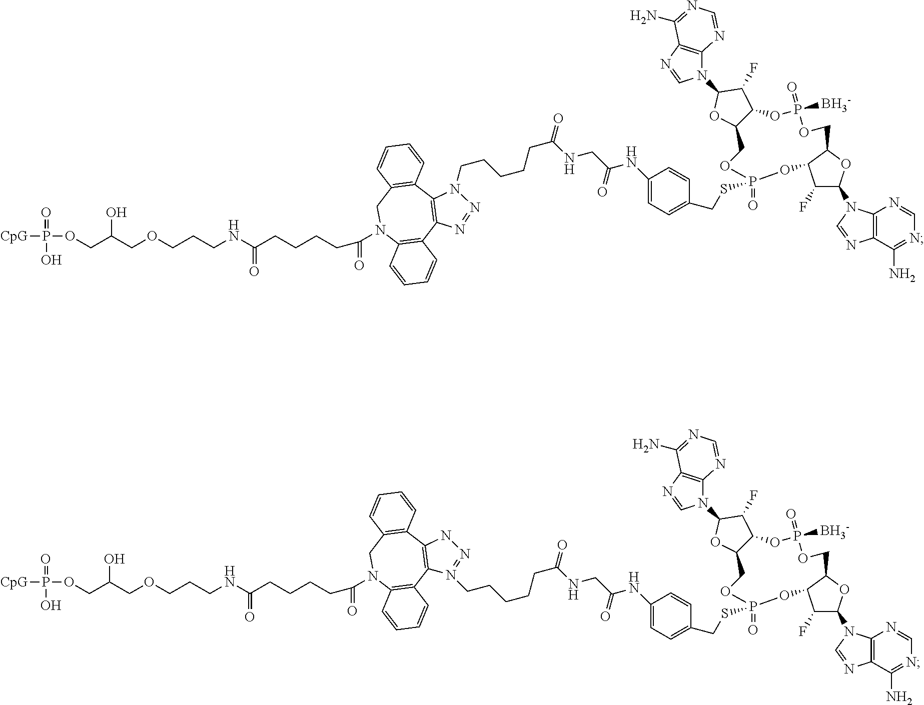

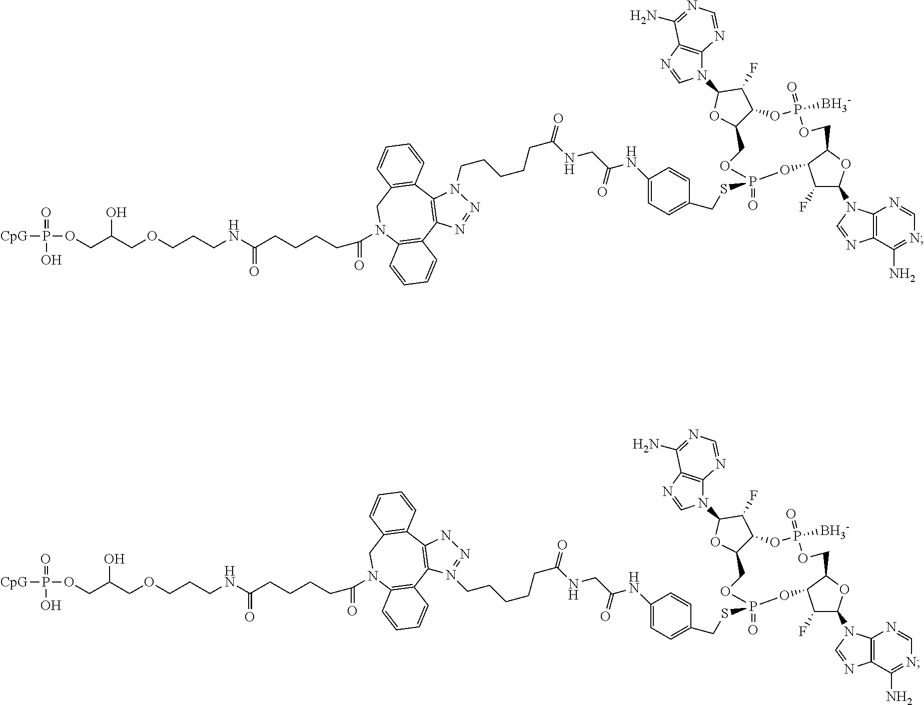

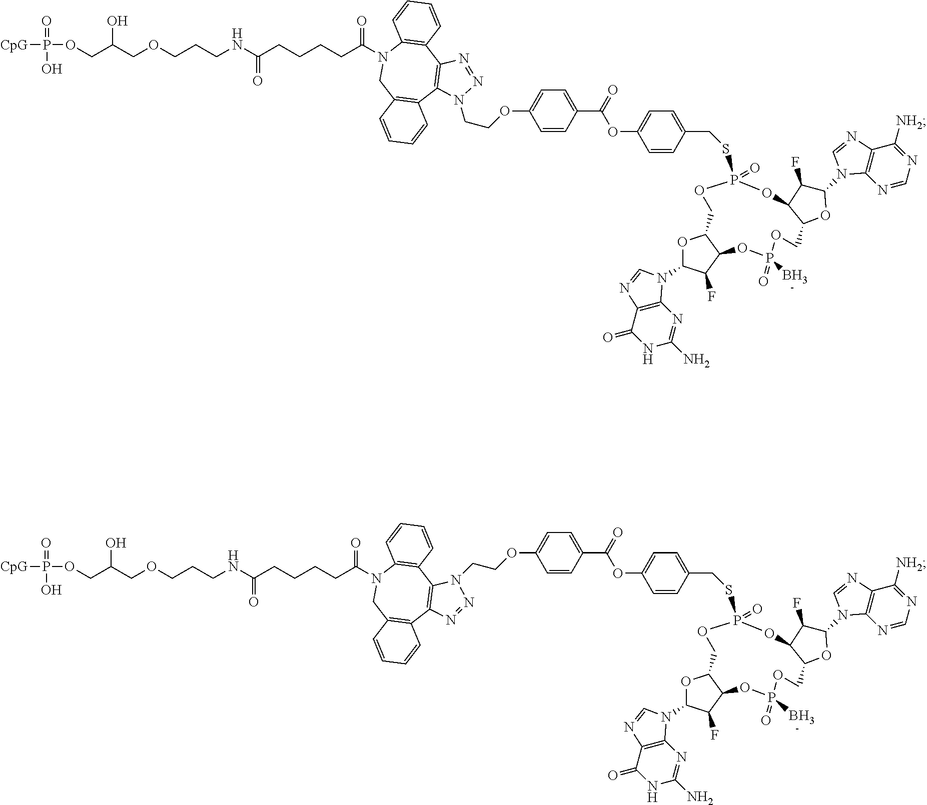

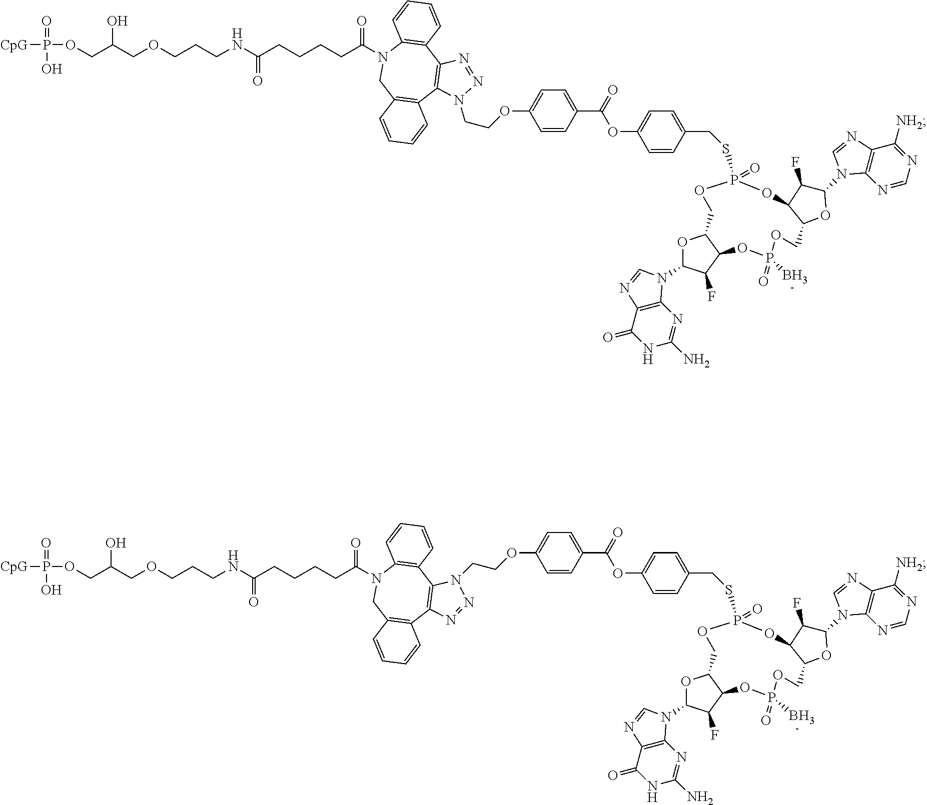

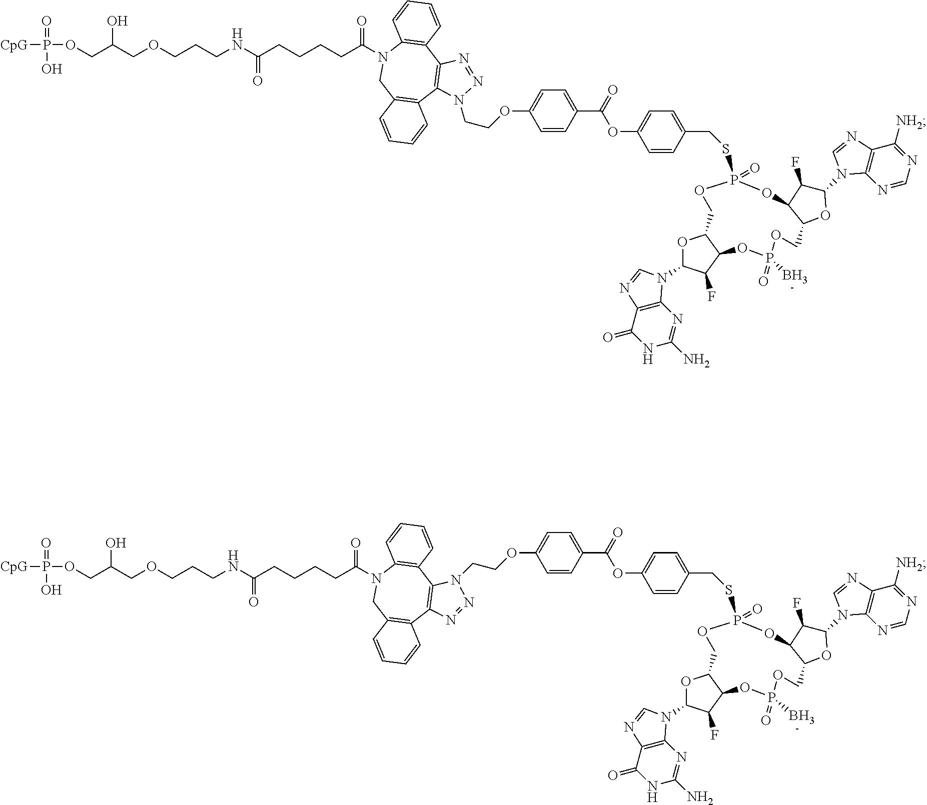

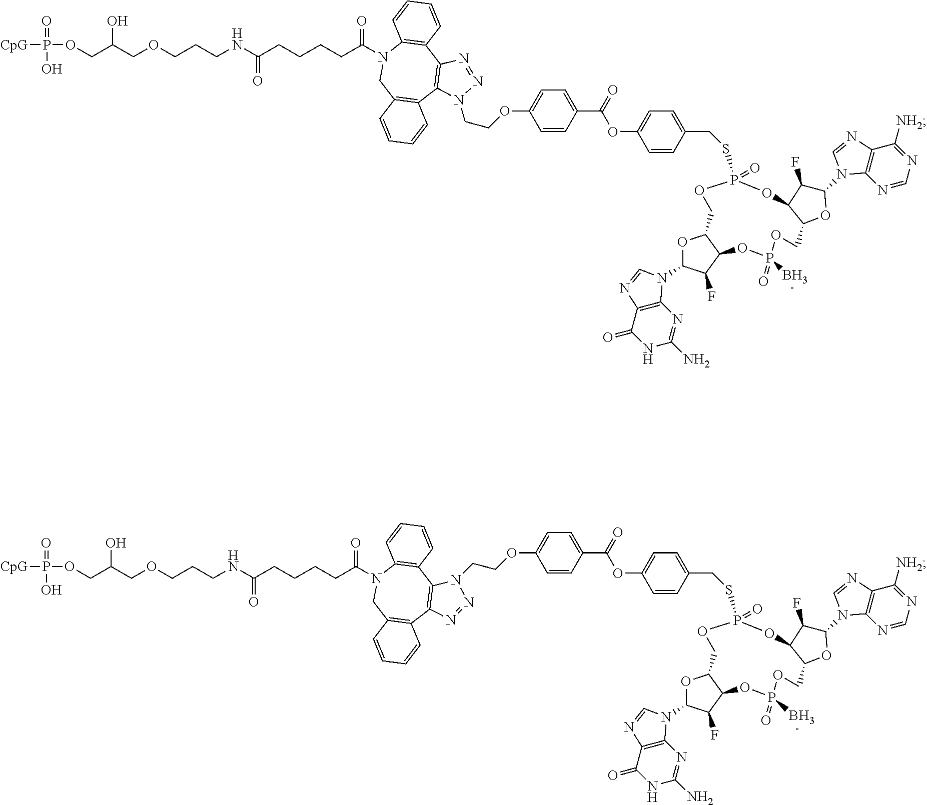

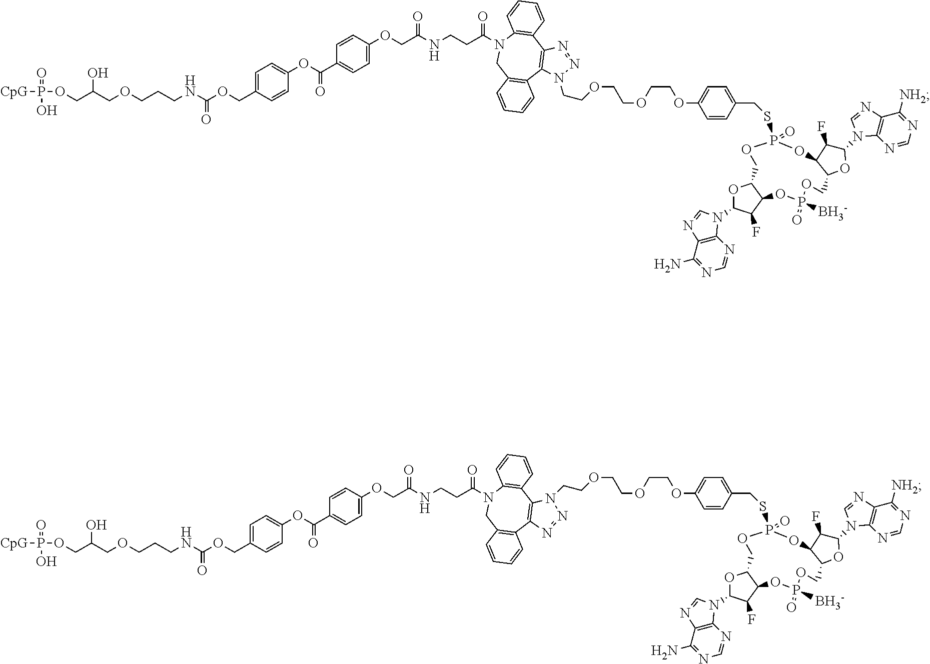

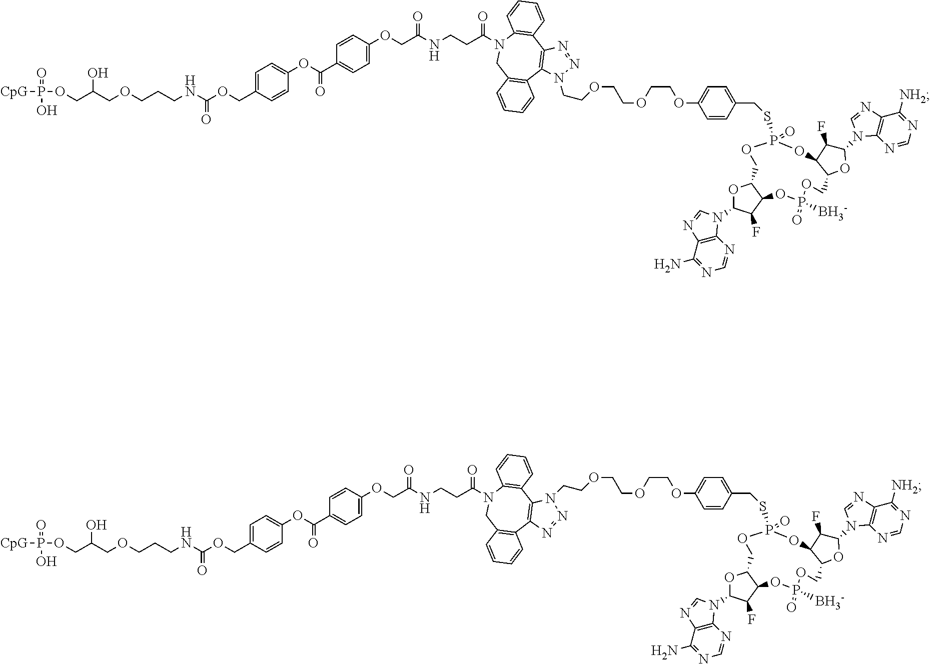

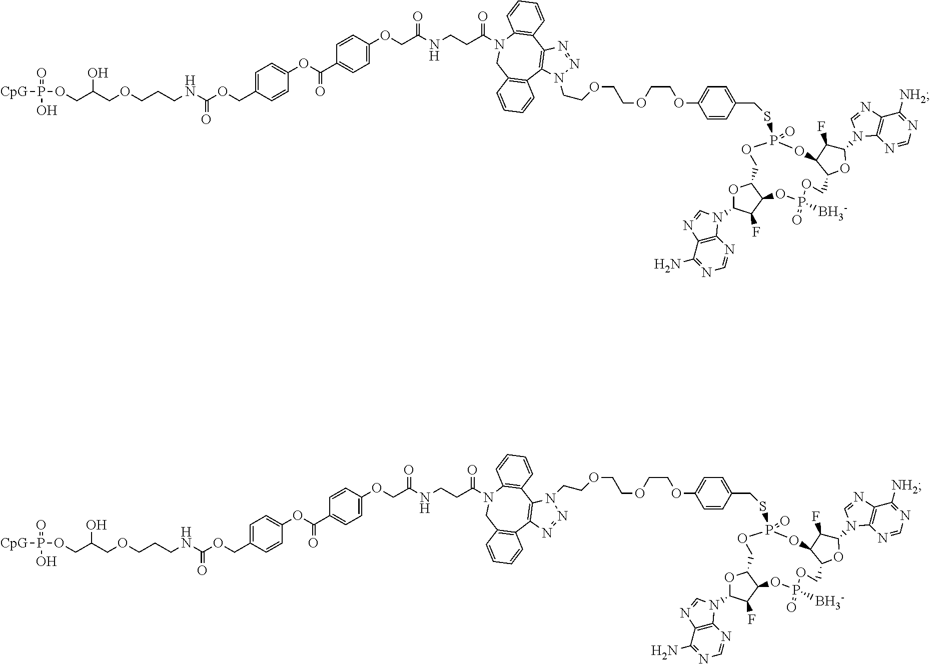

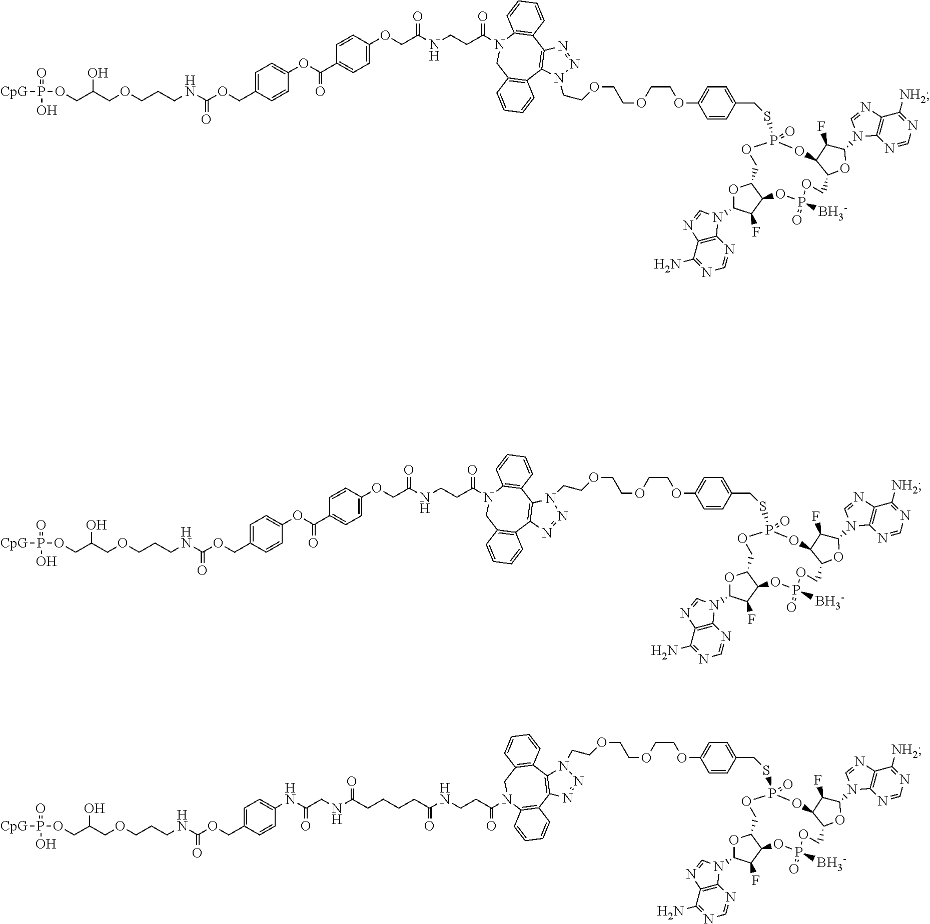

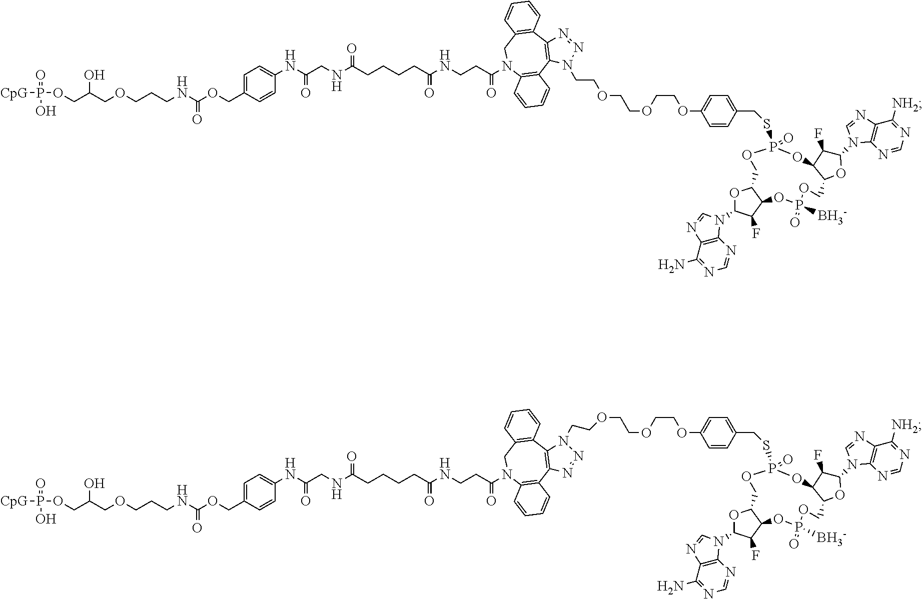

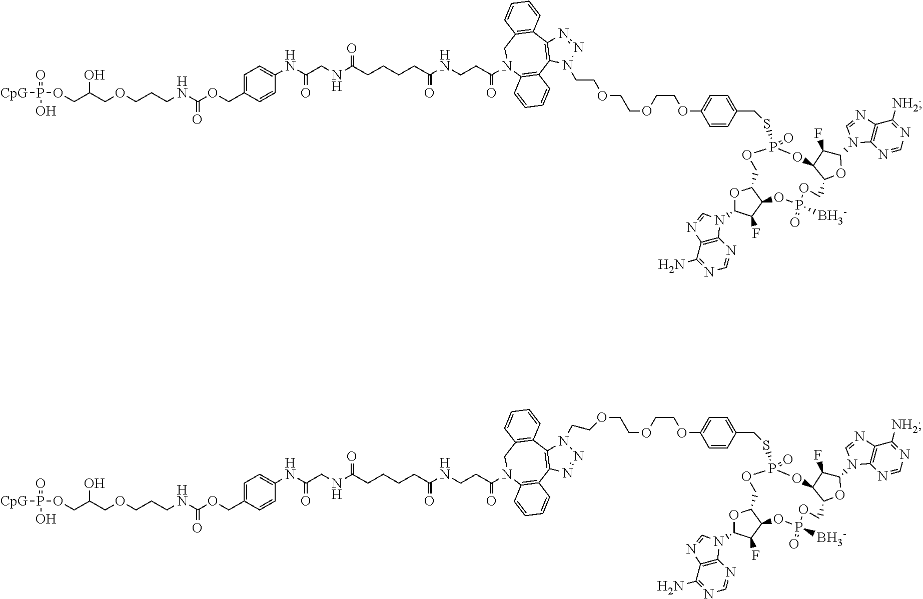

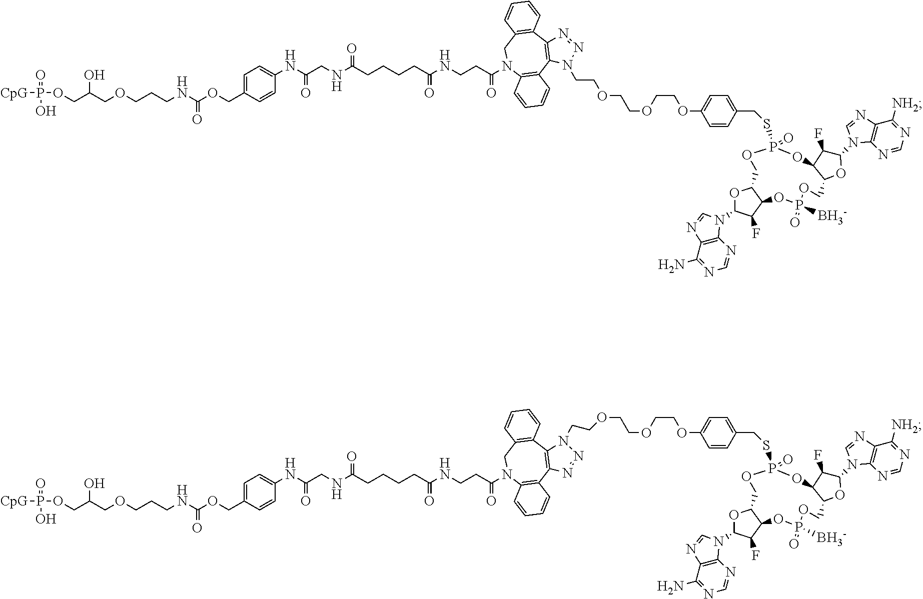

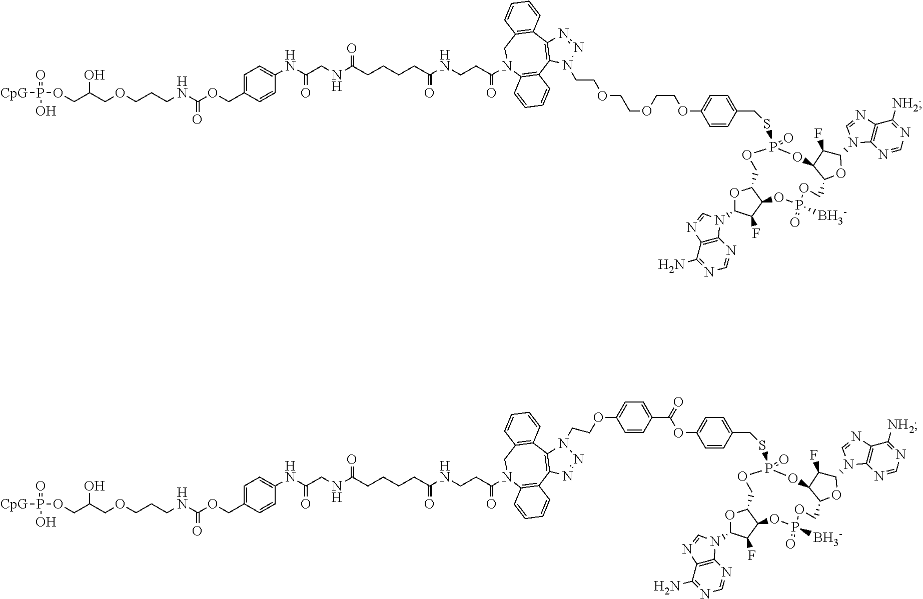

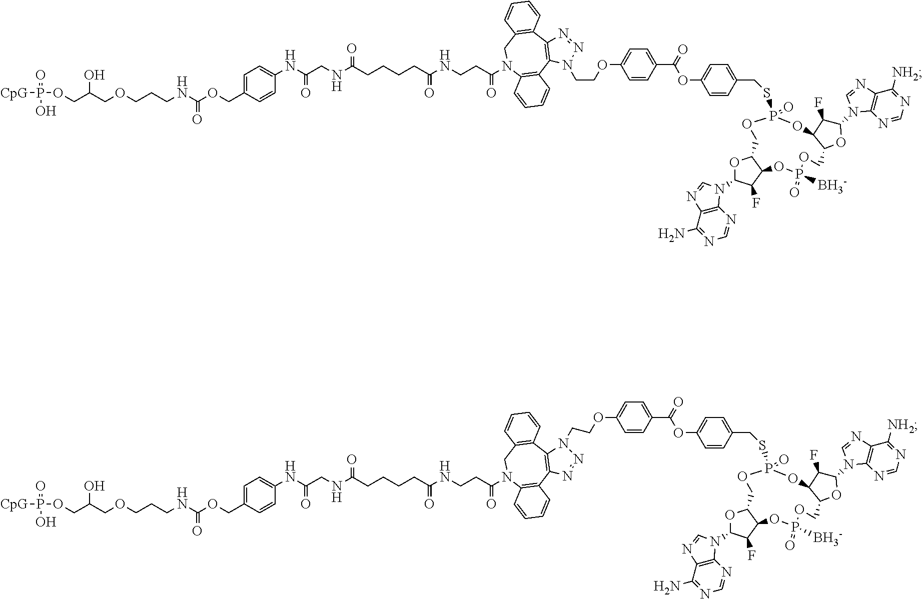

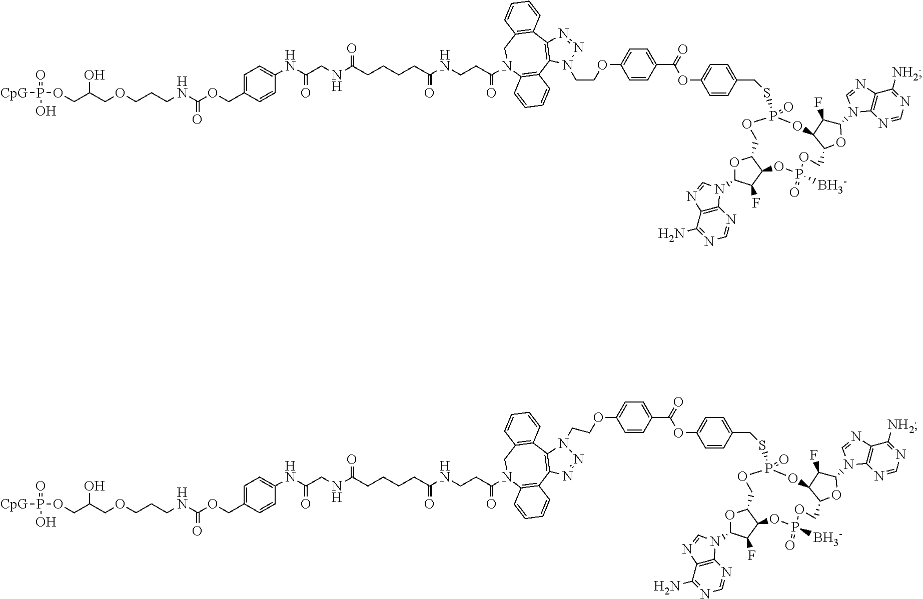

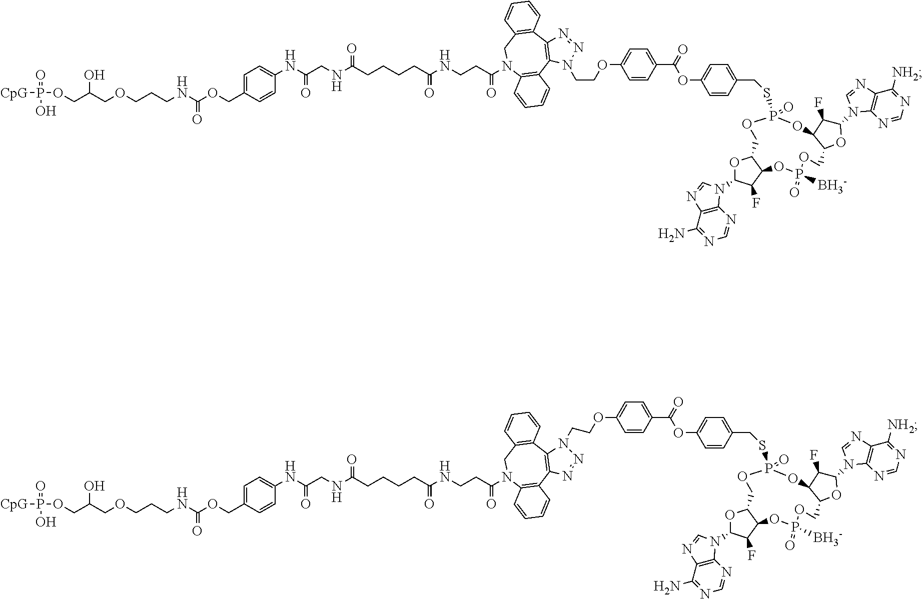

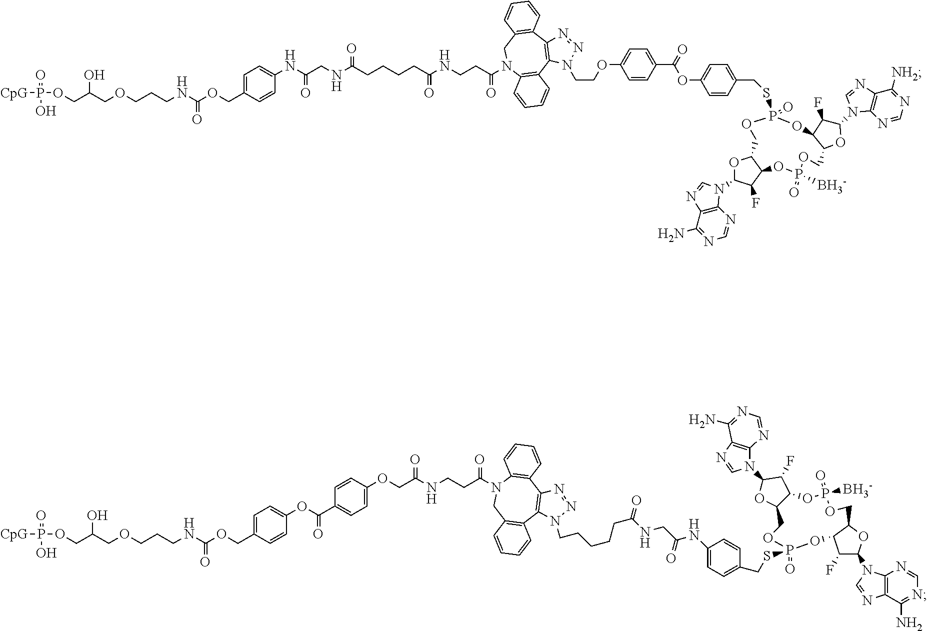

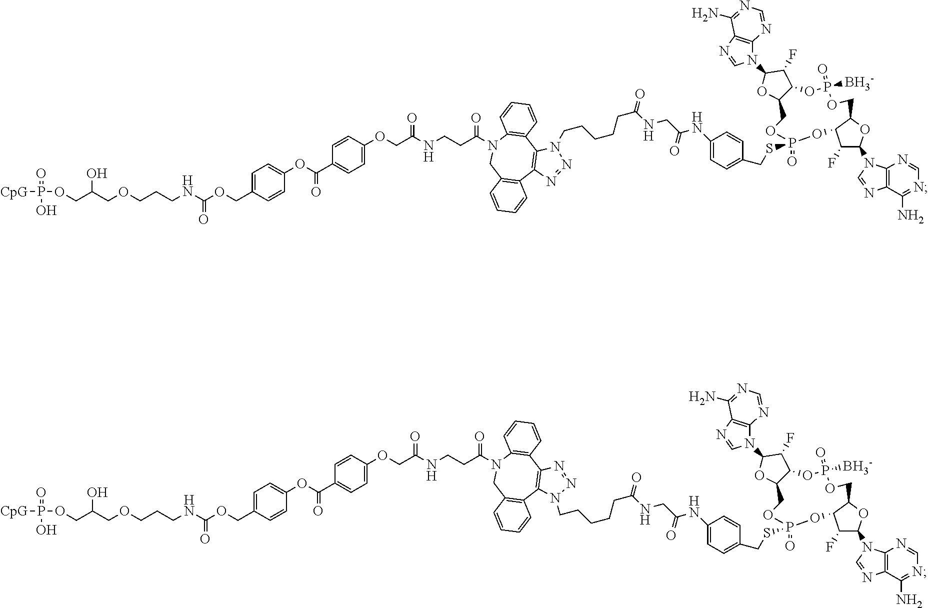

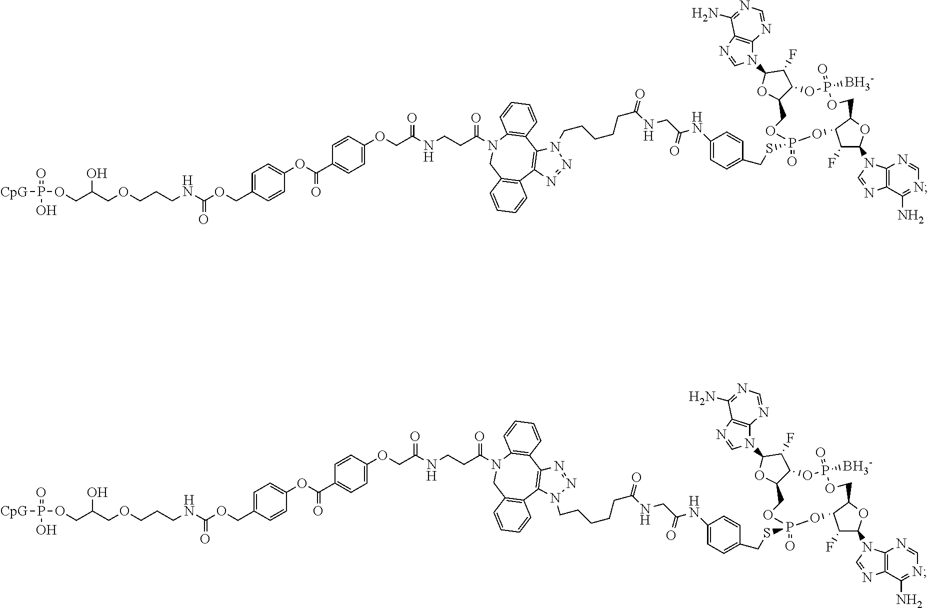

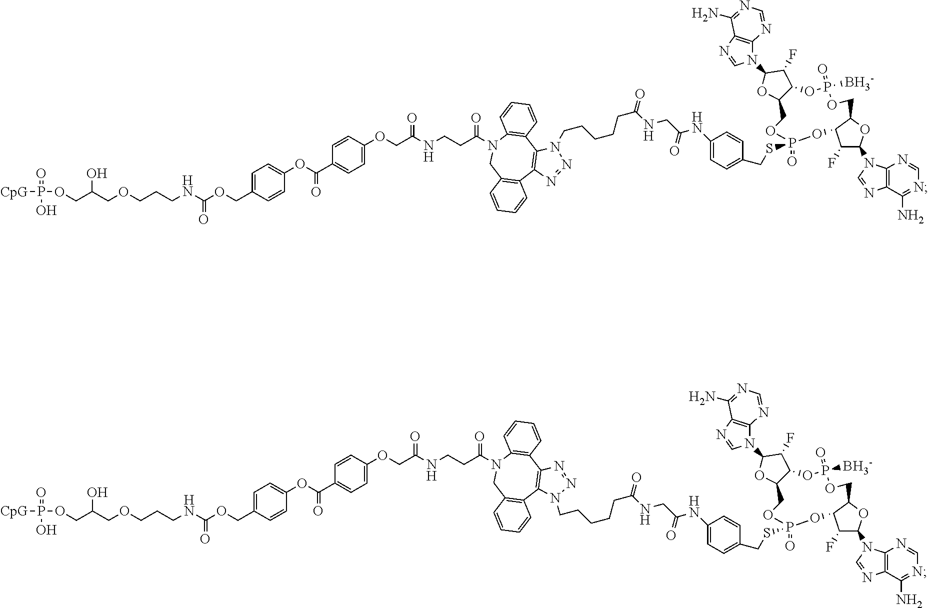

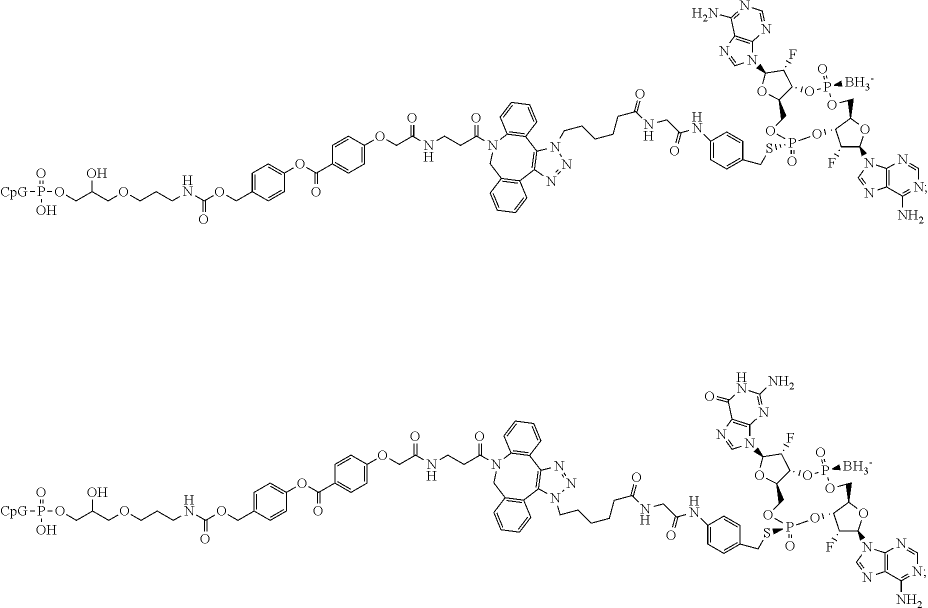

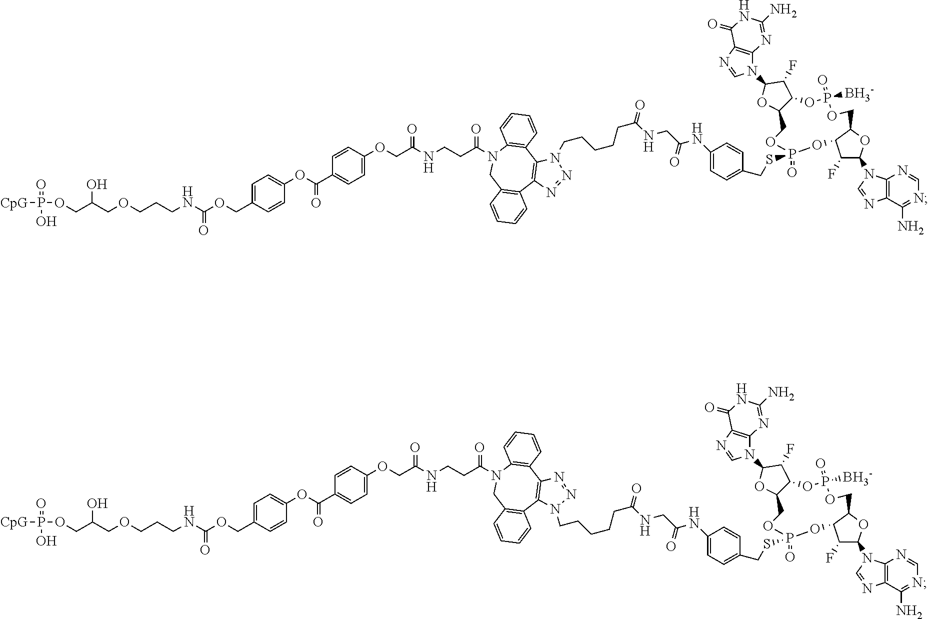

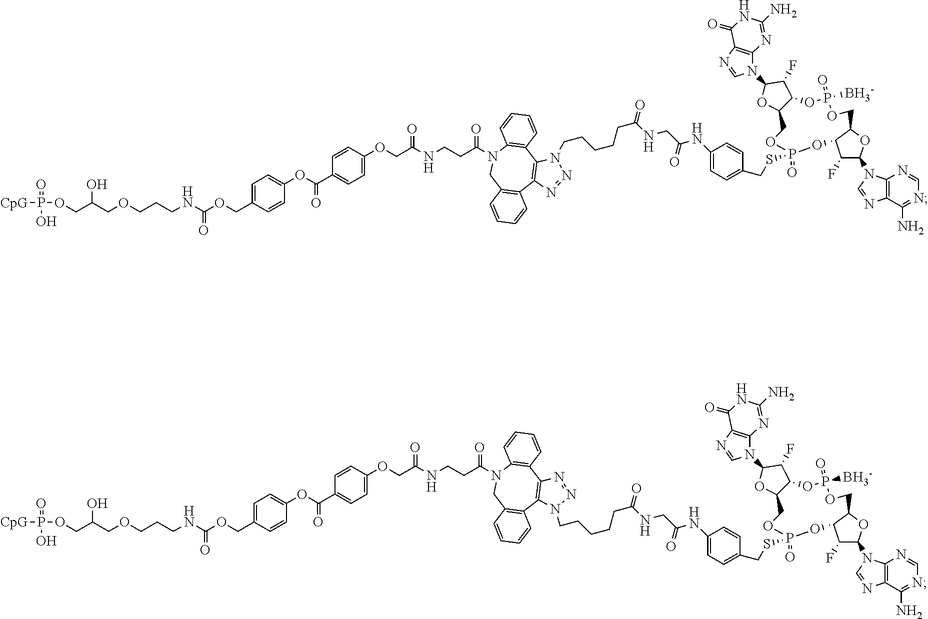

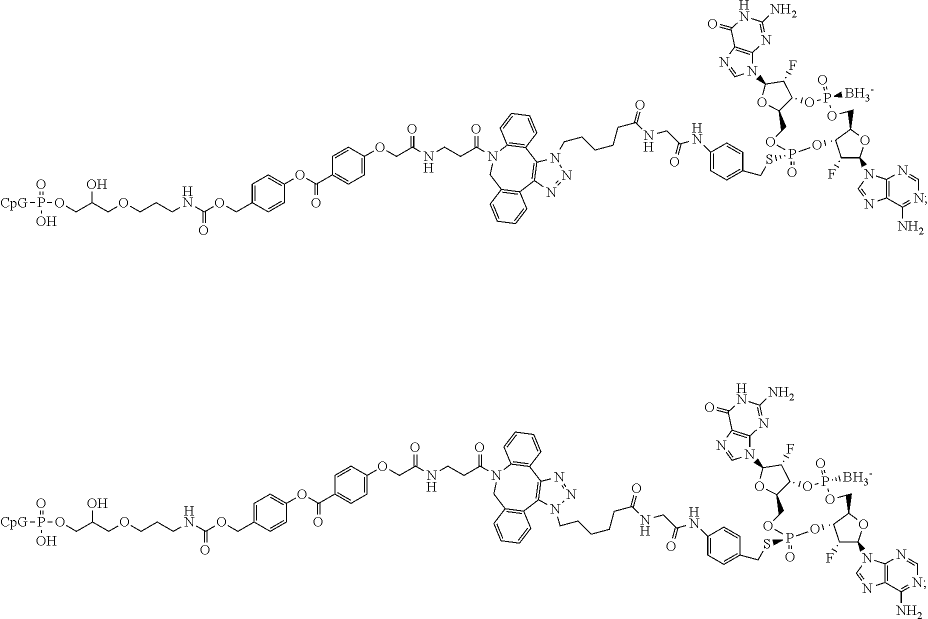

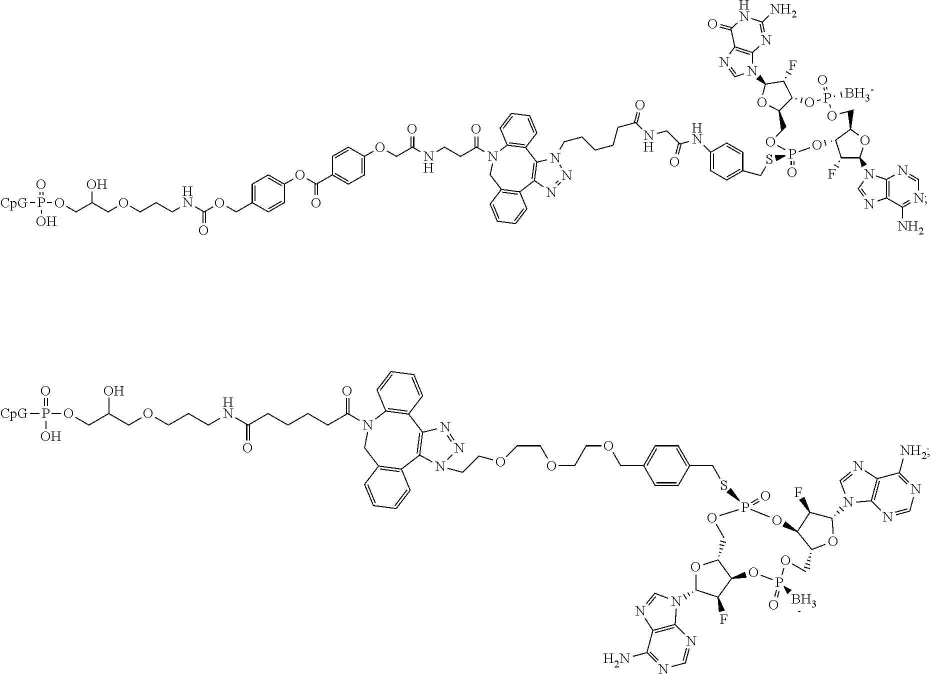

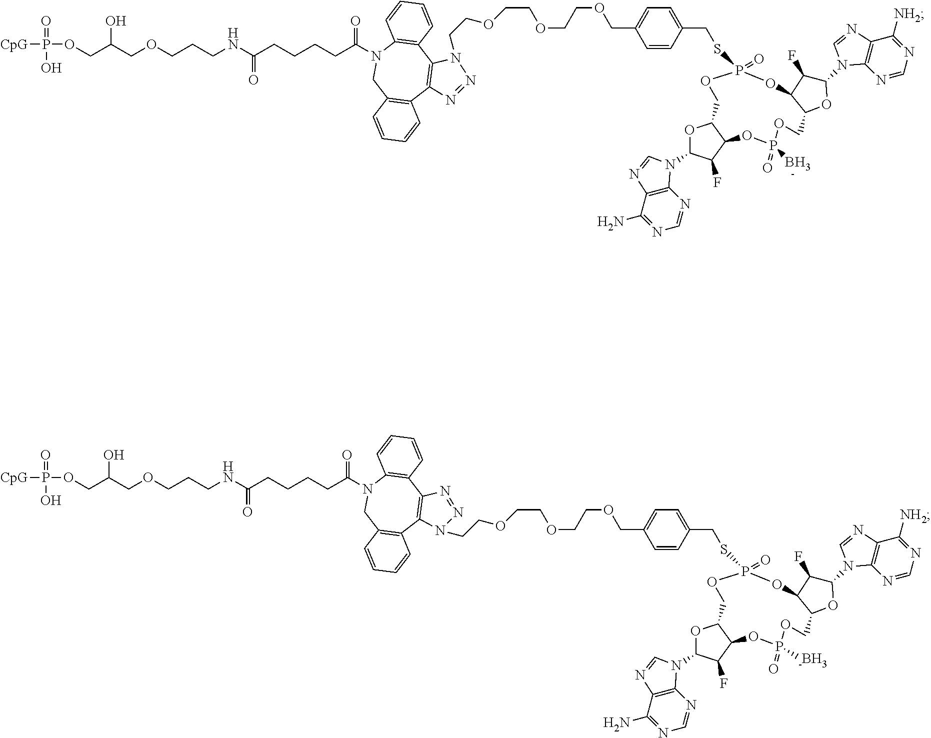

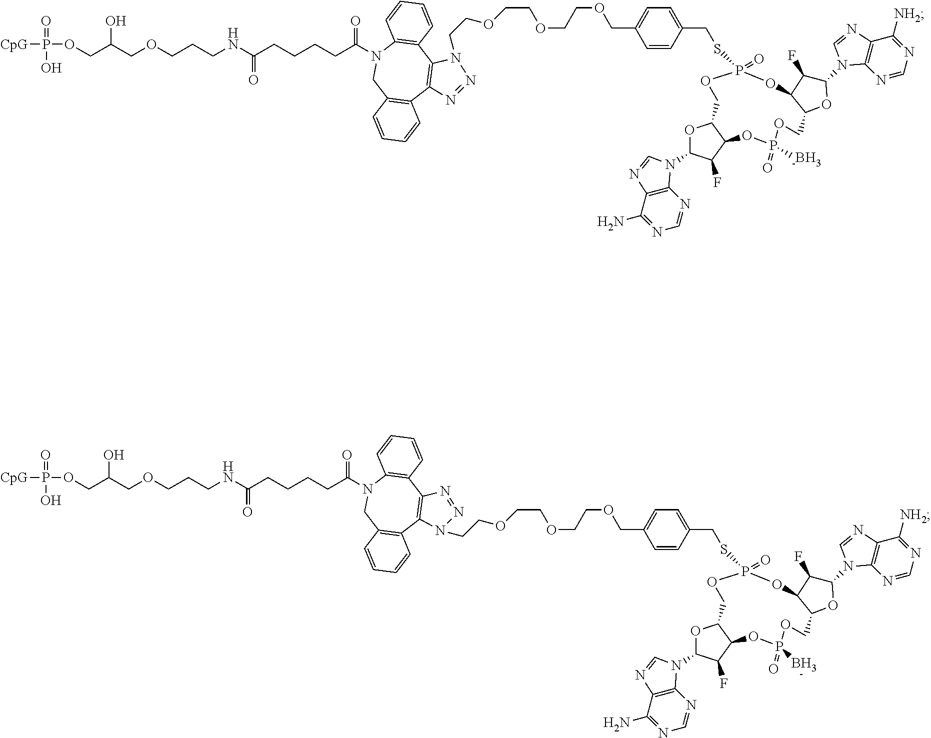

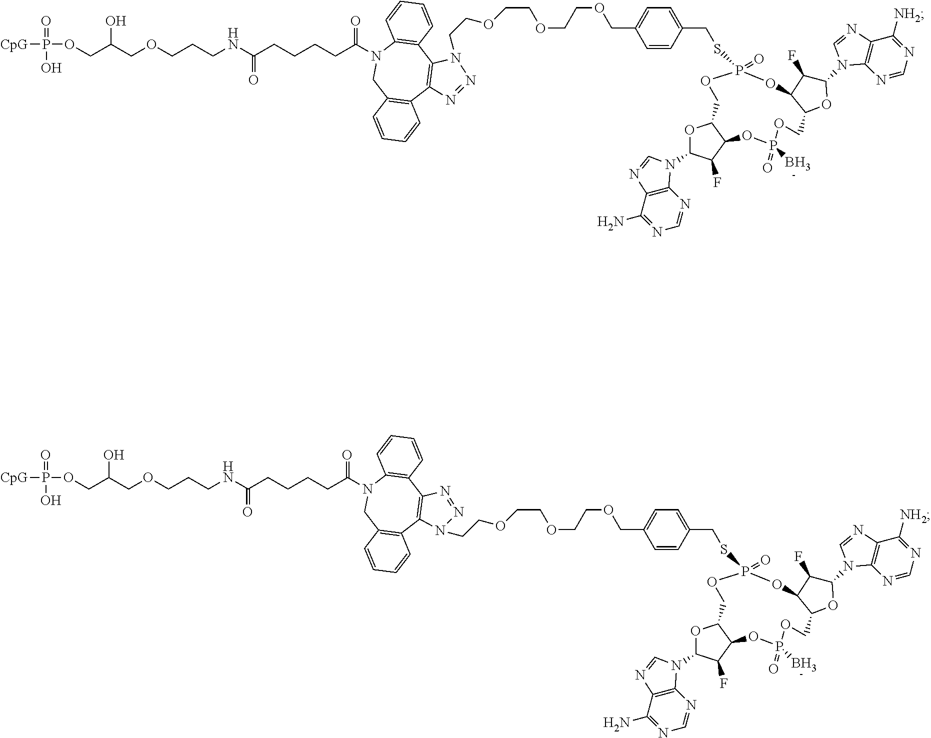

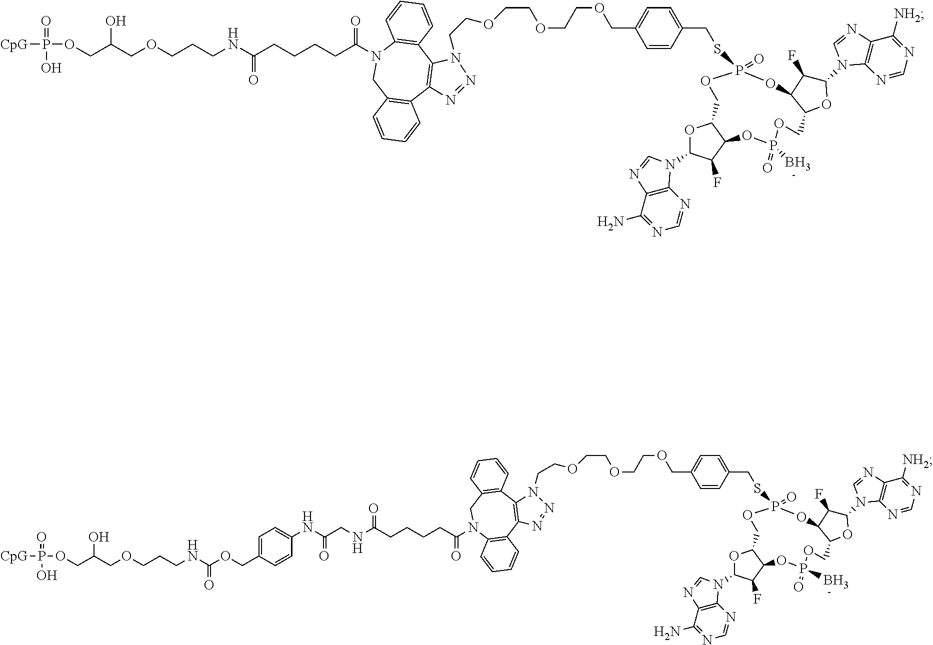

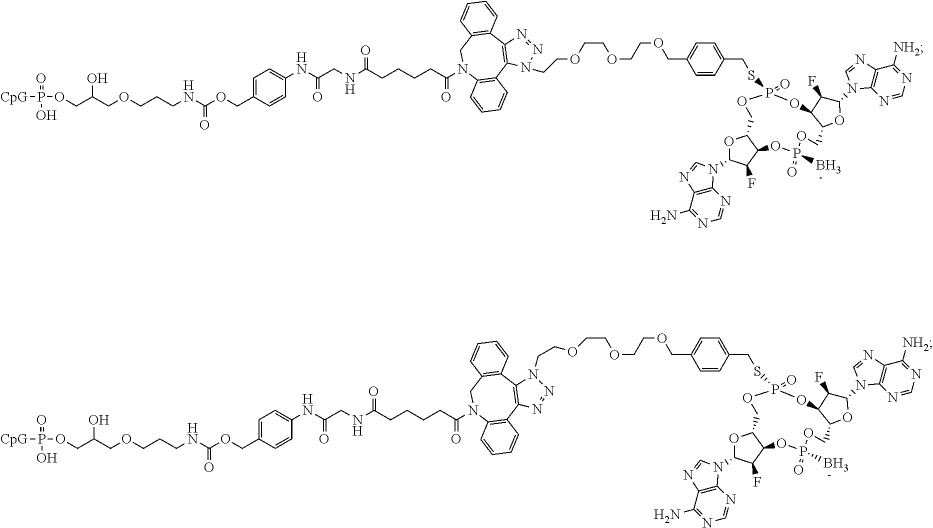

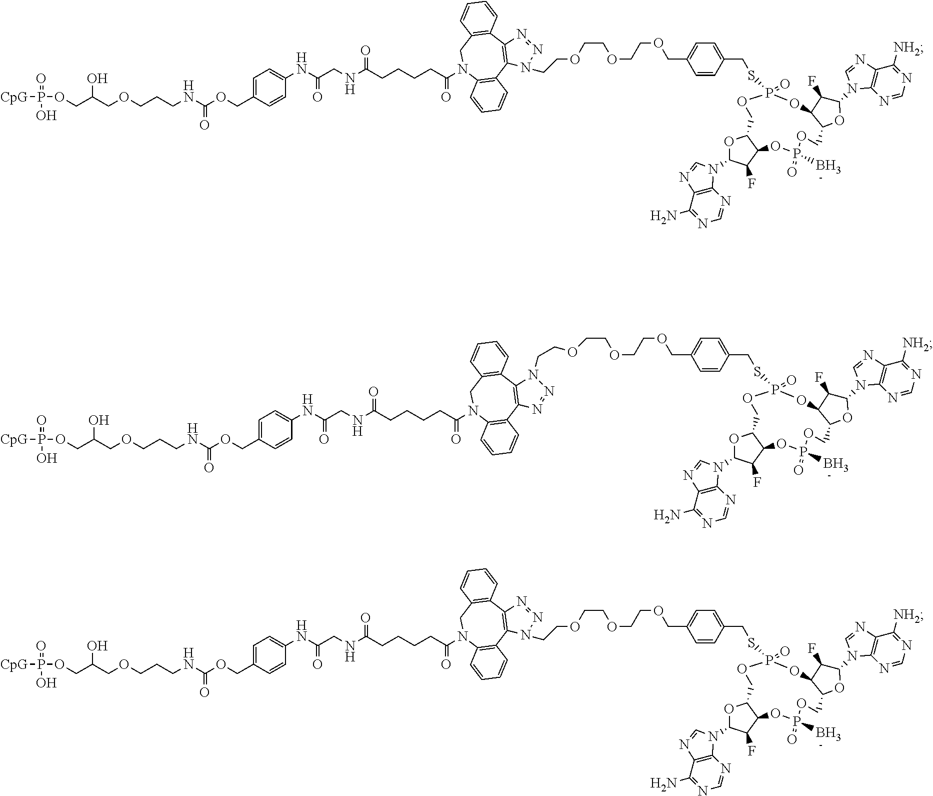

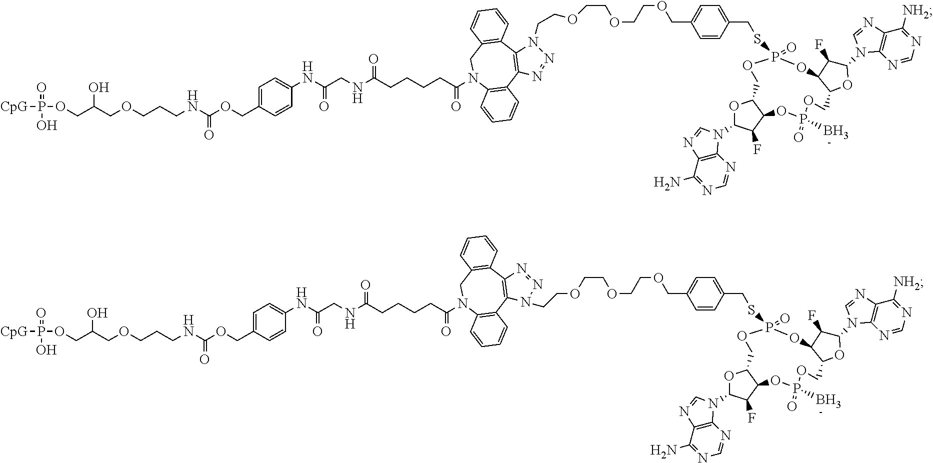

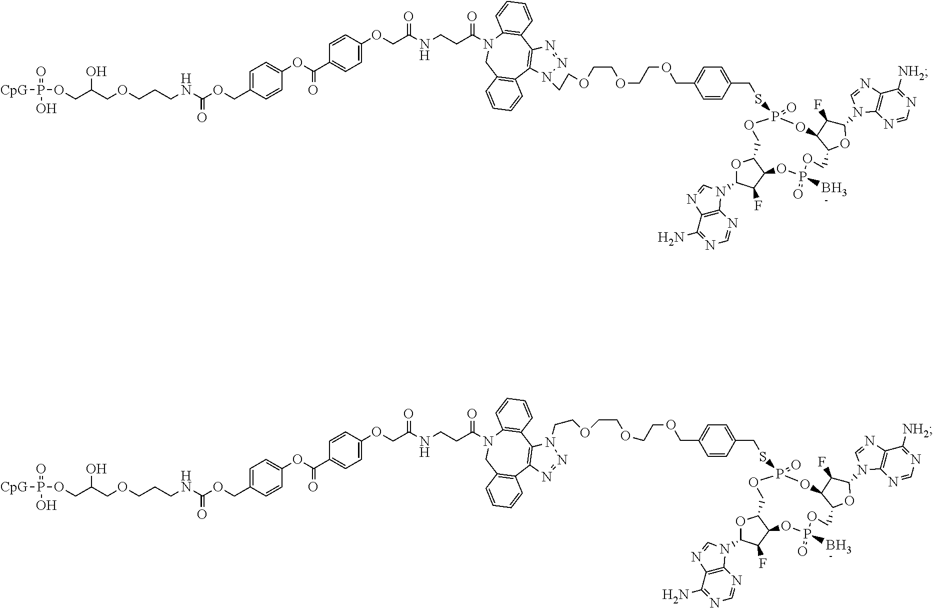

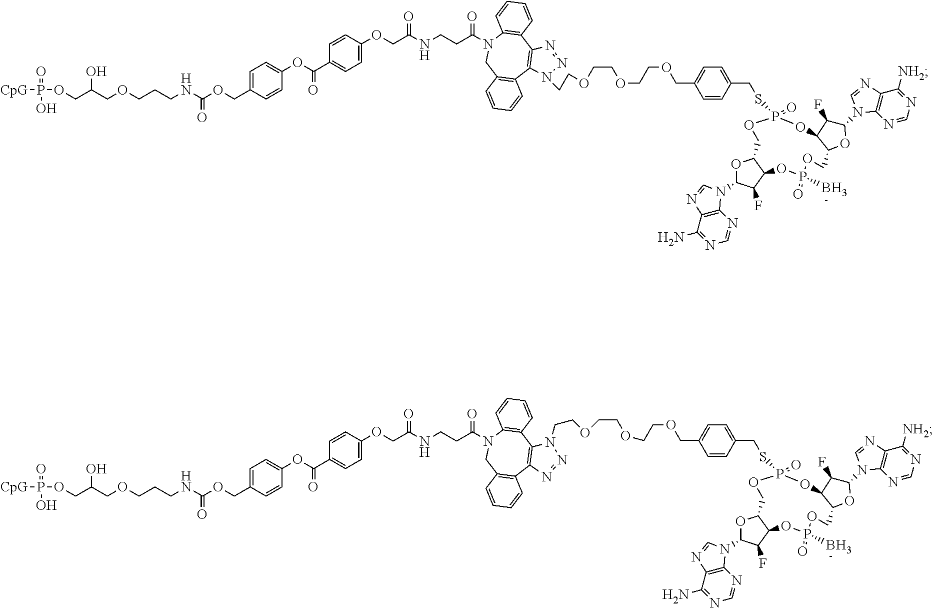

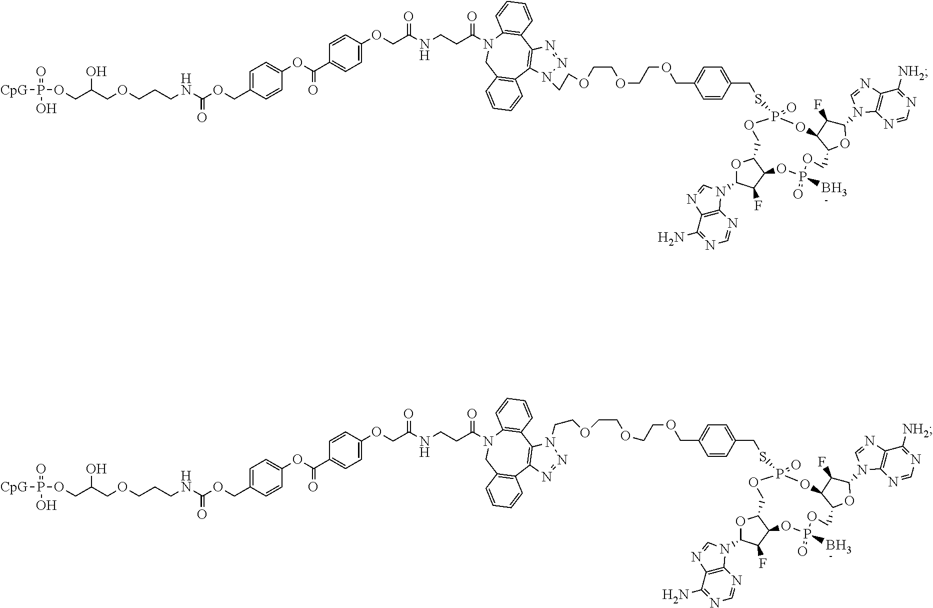

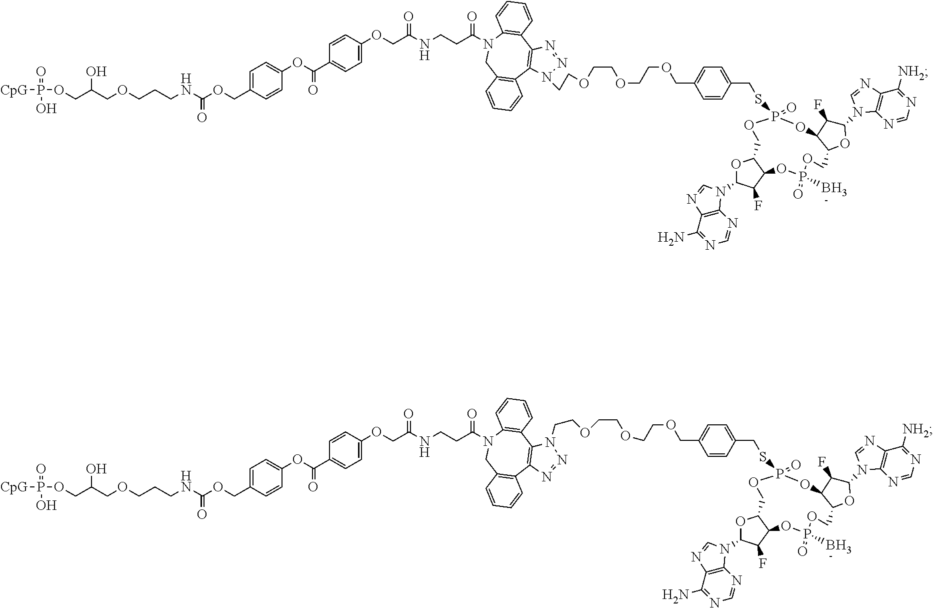

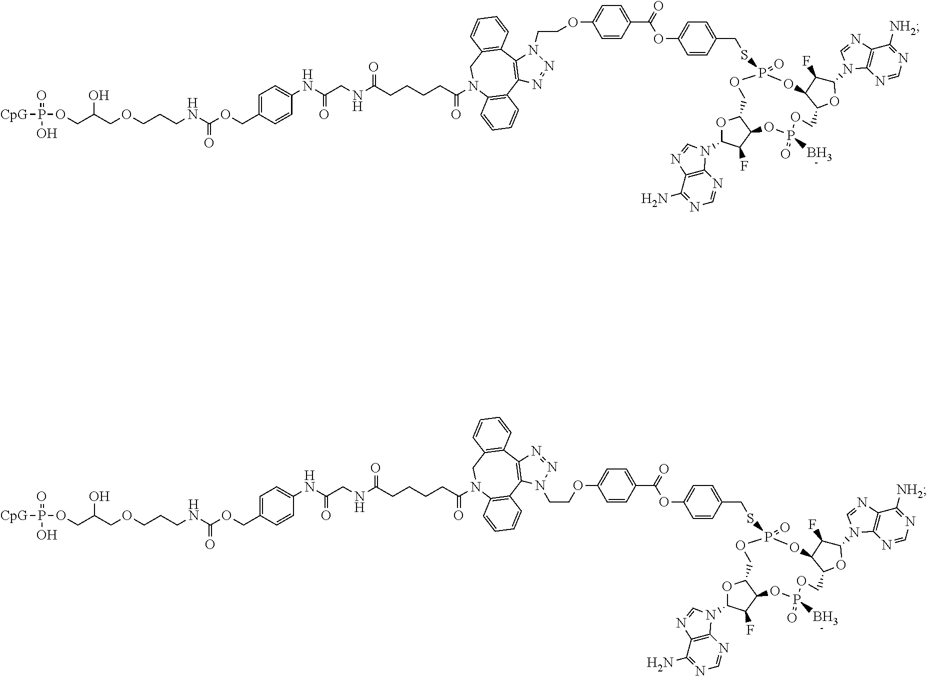

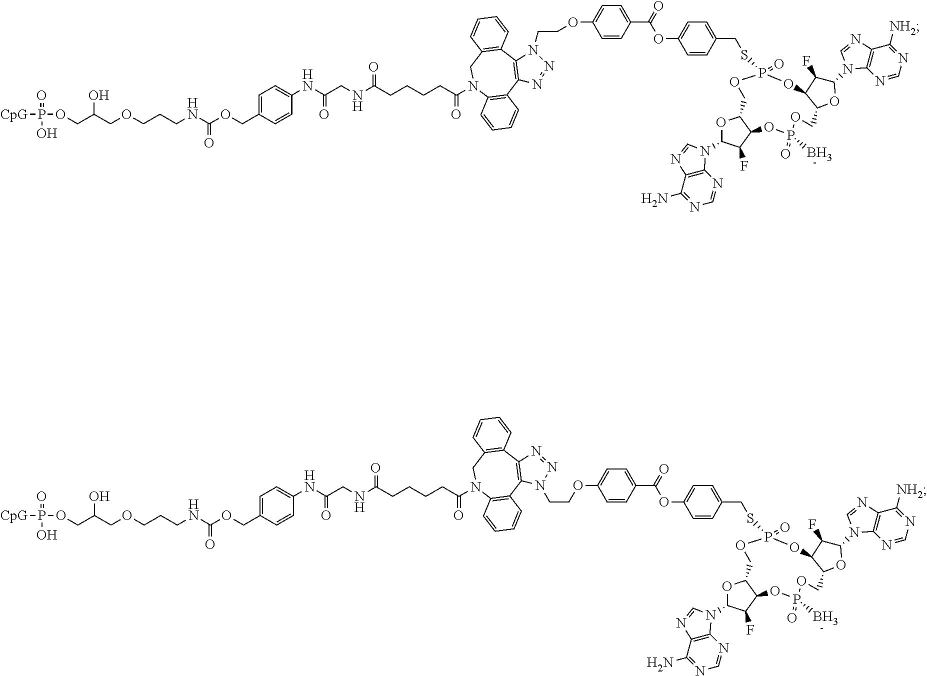

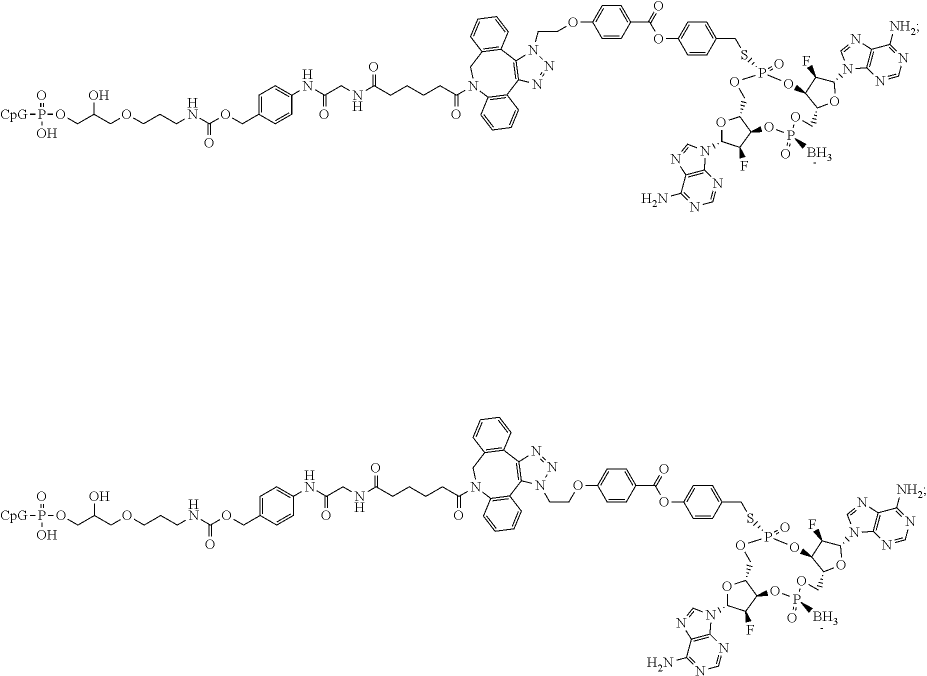

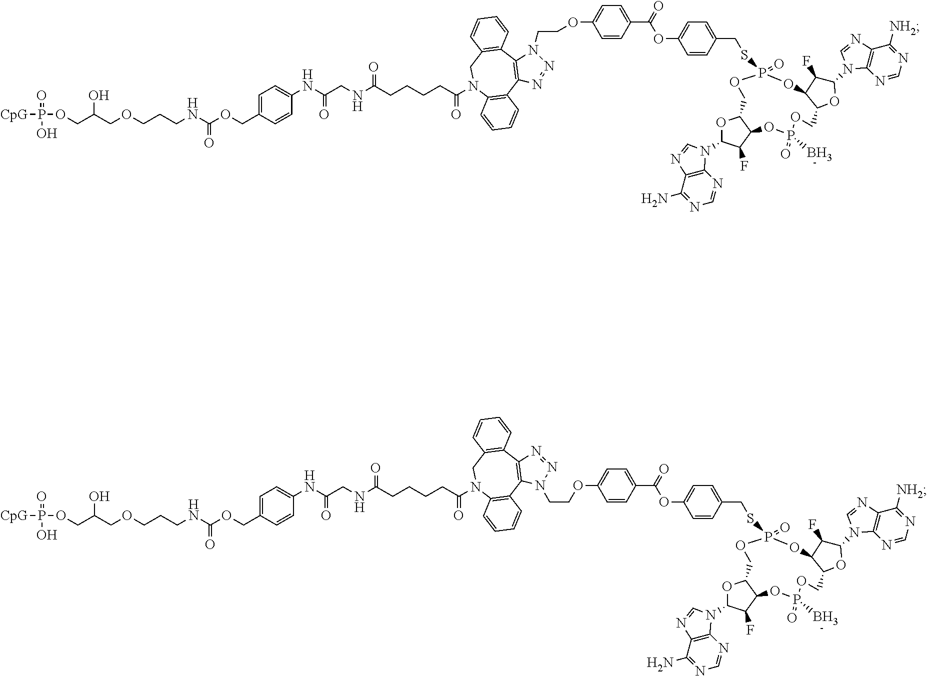

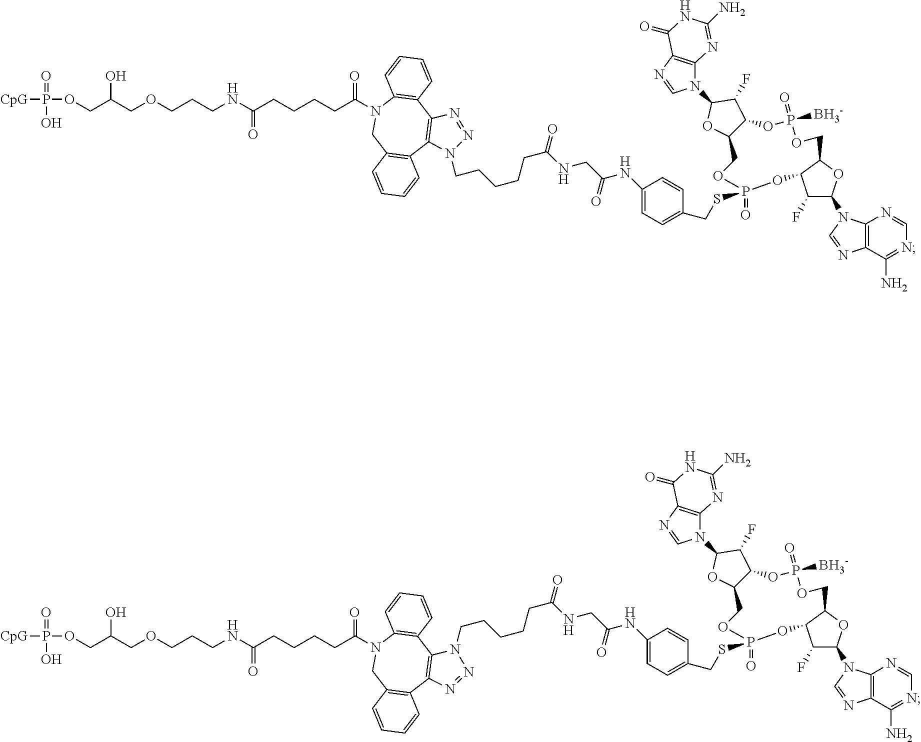

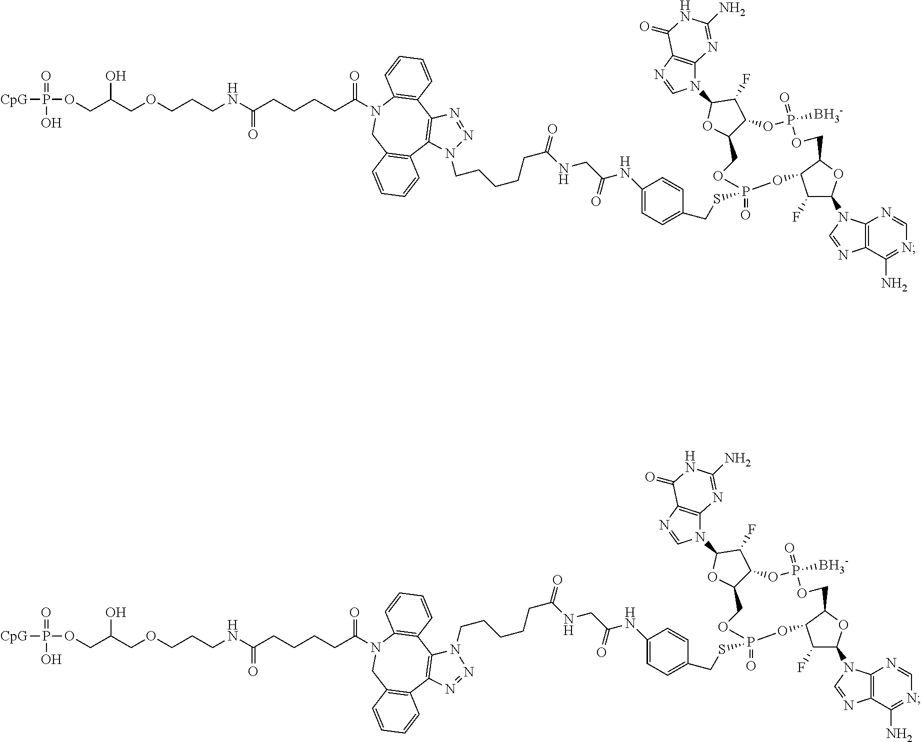

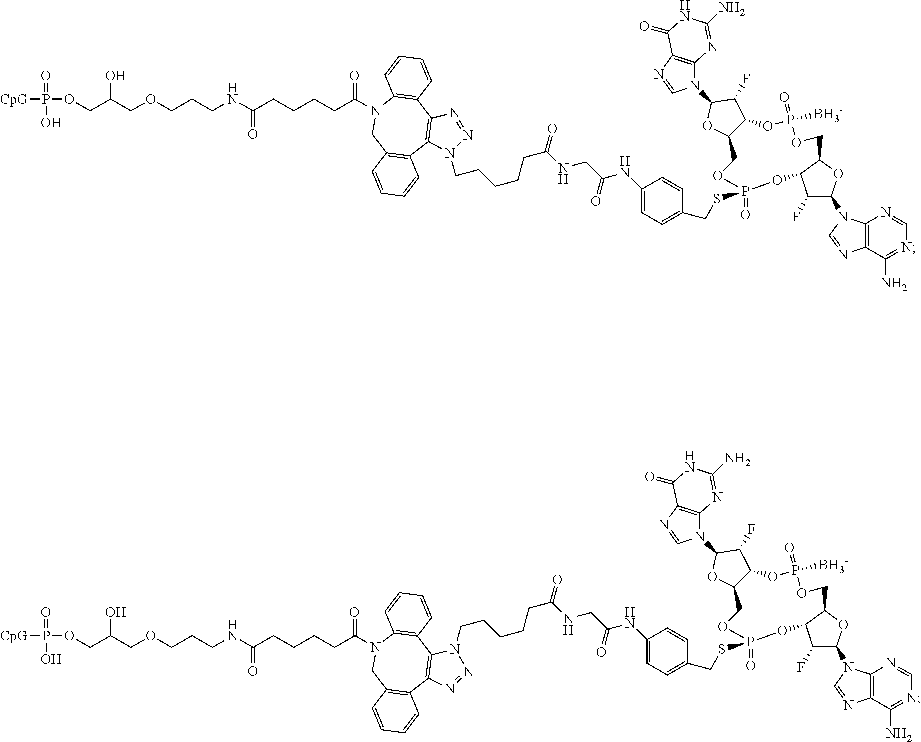

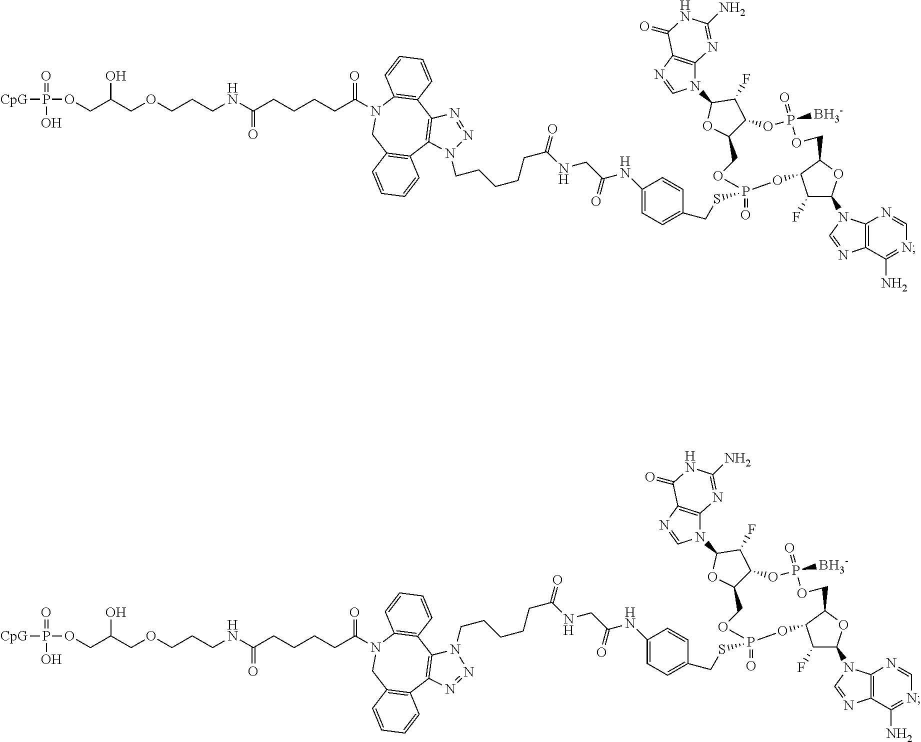

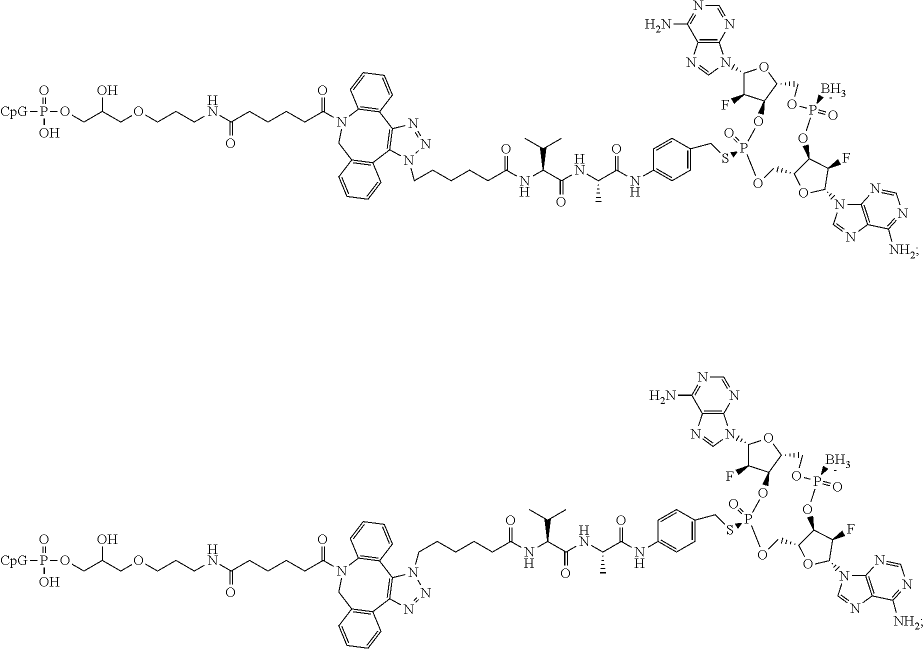

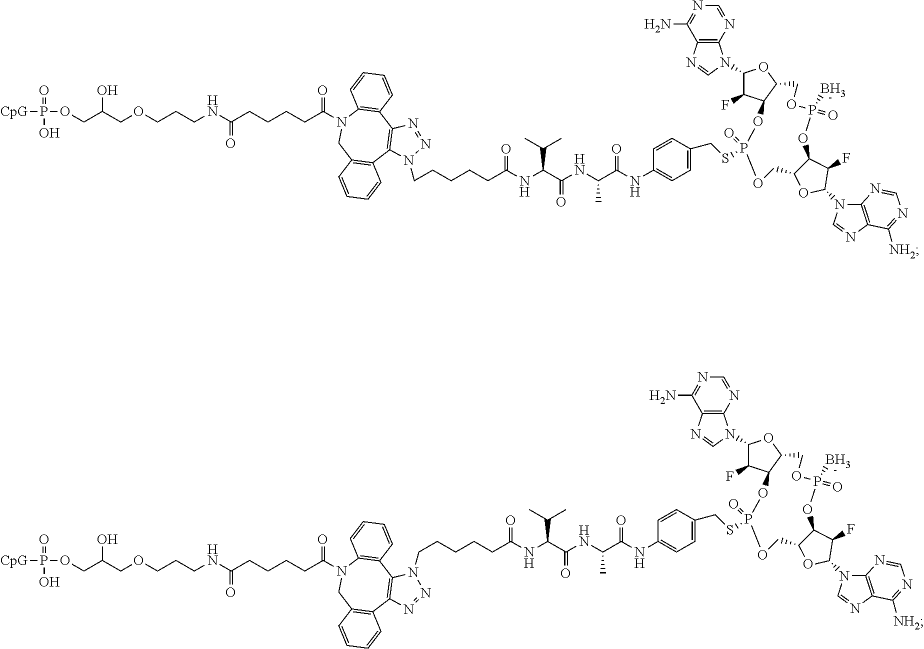

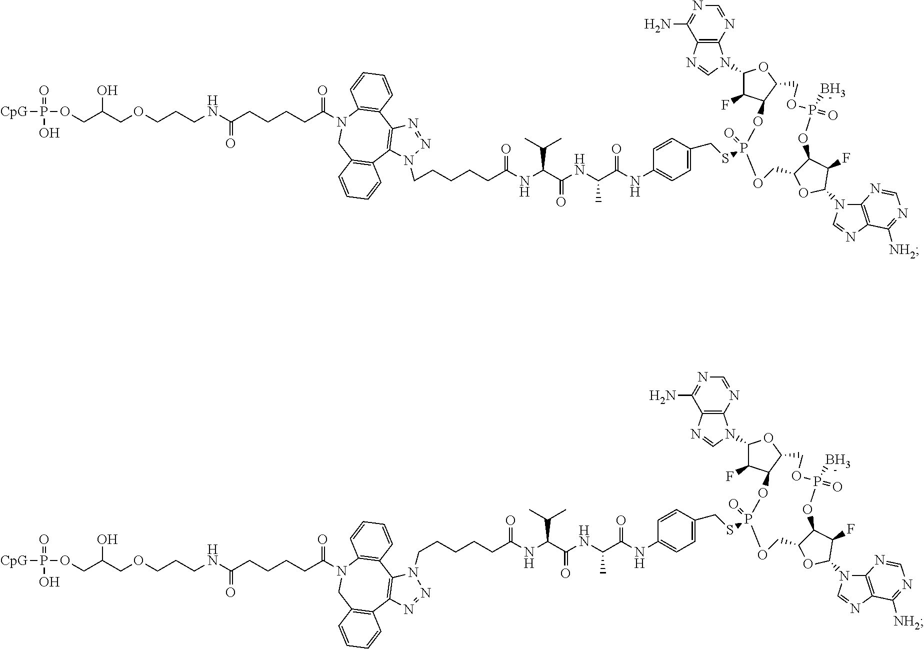

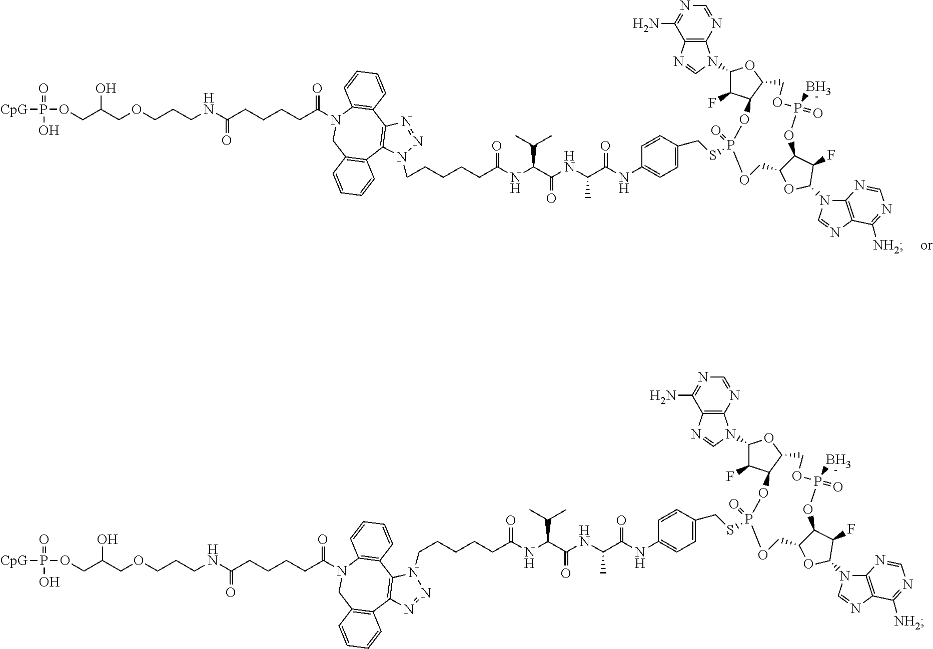

or a stereoisomer thereof;
wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide; and

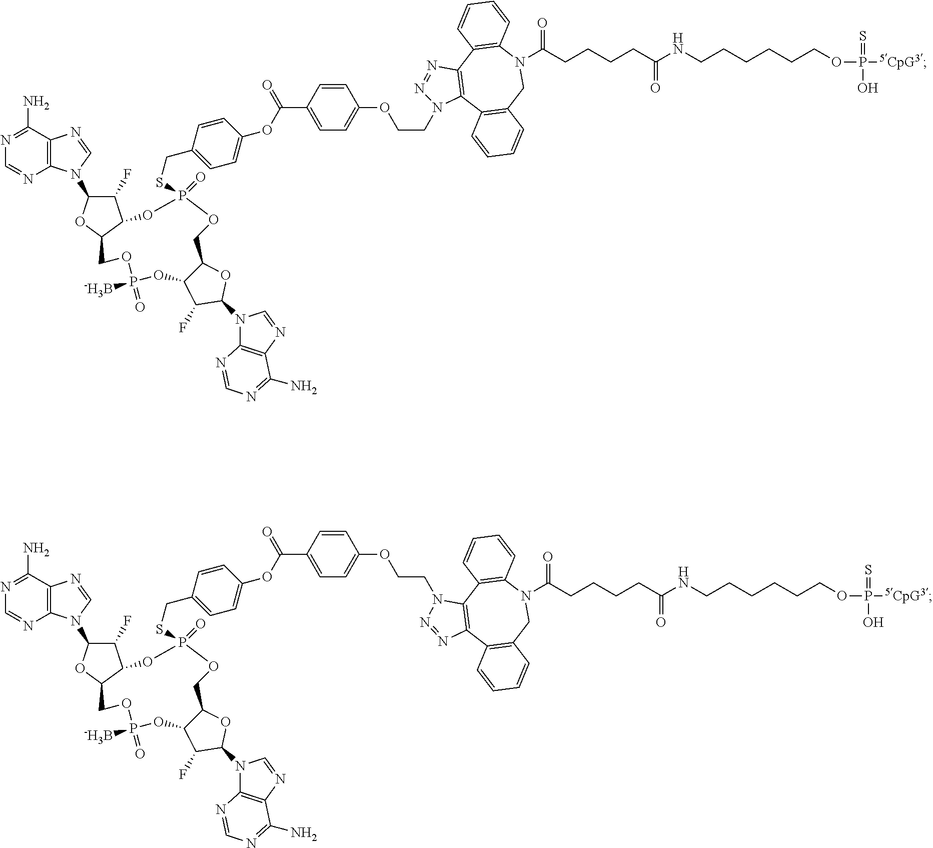

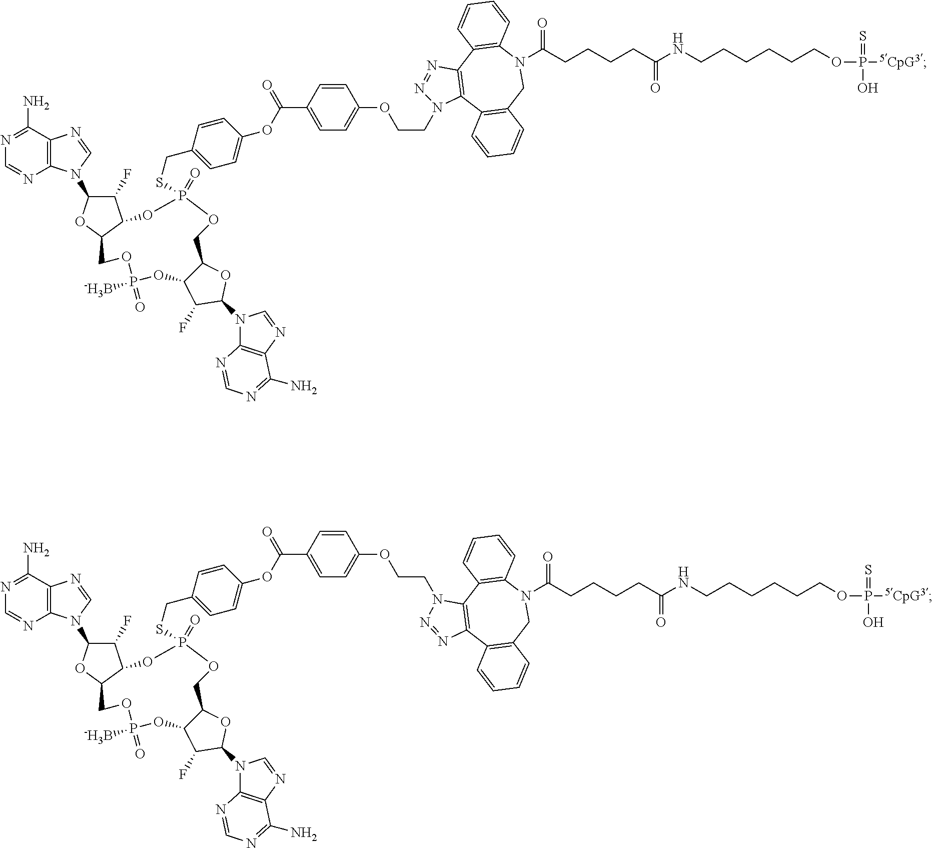

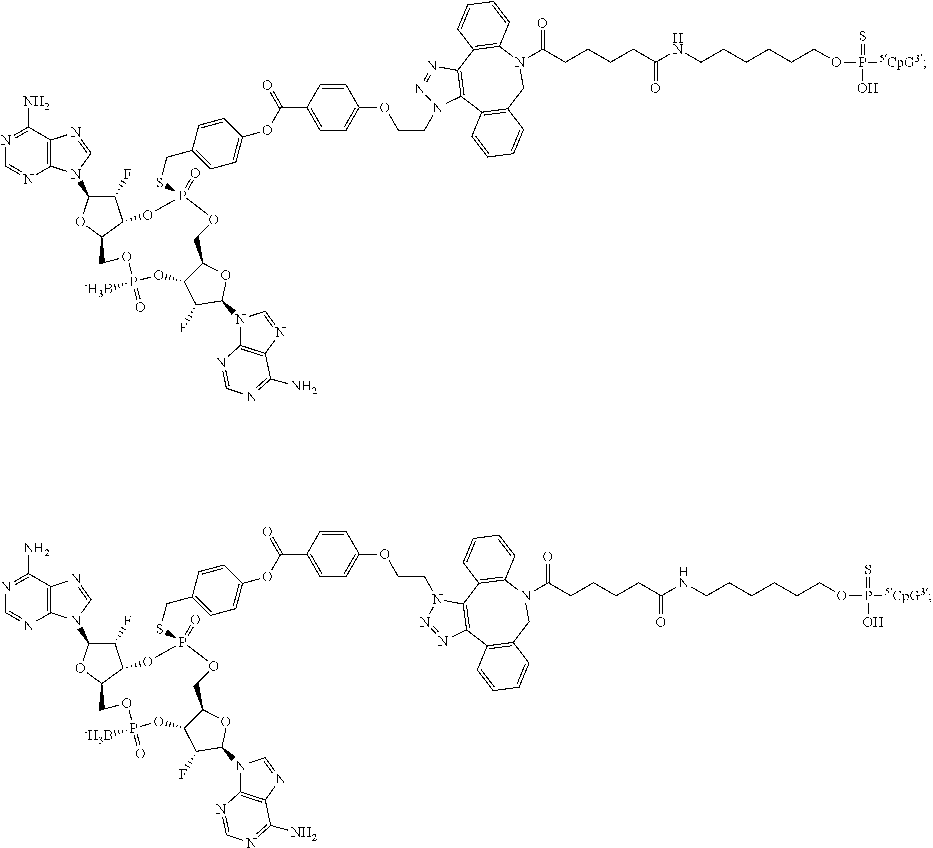

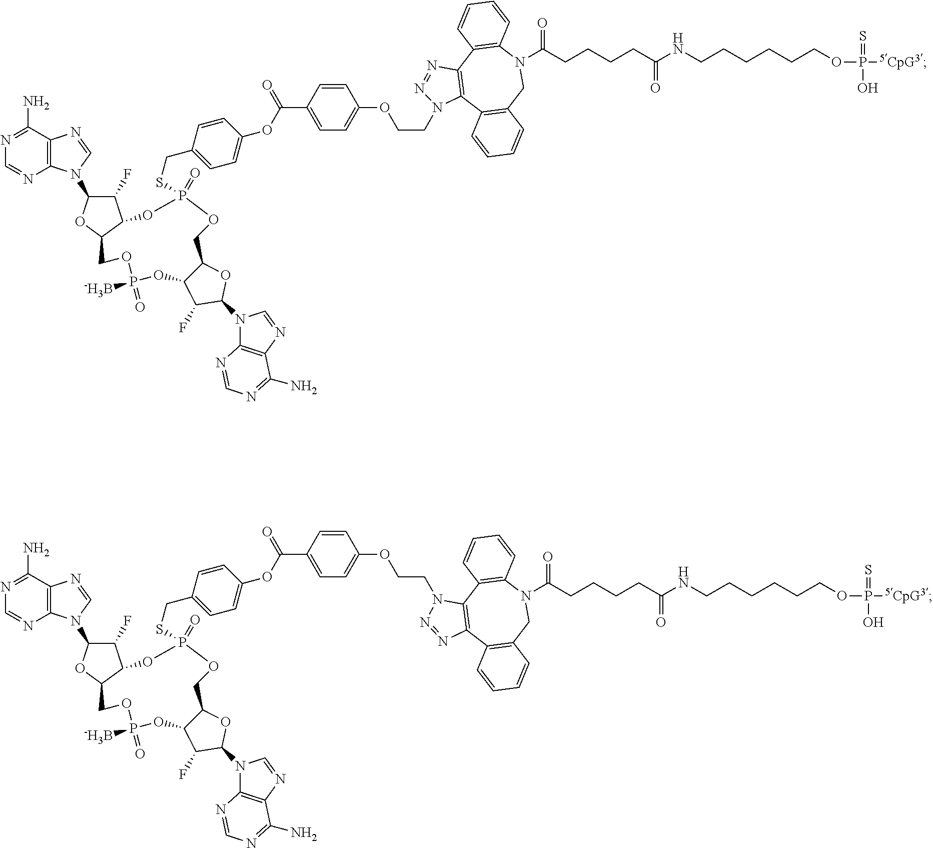

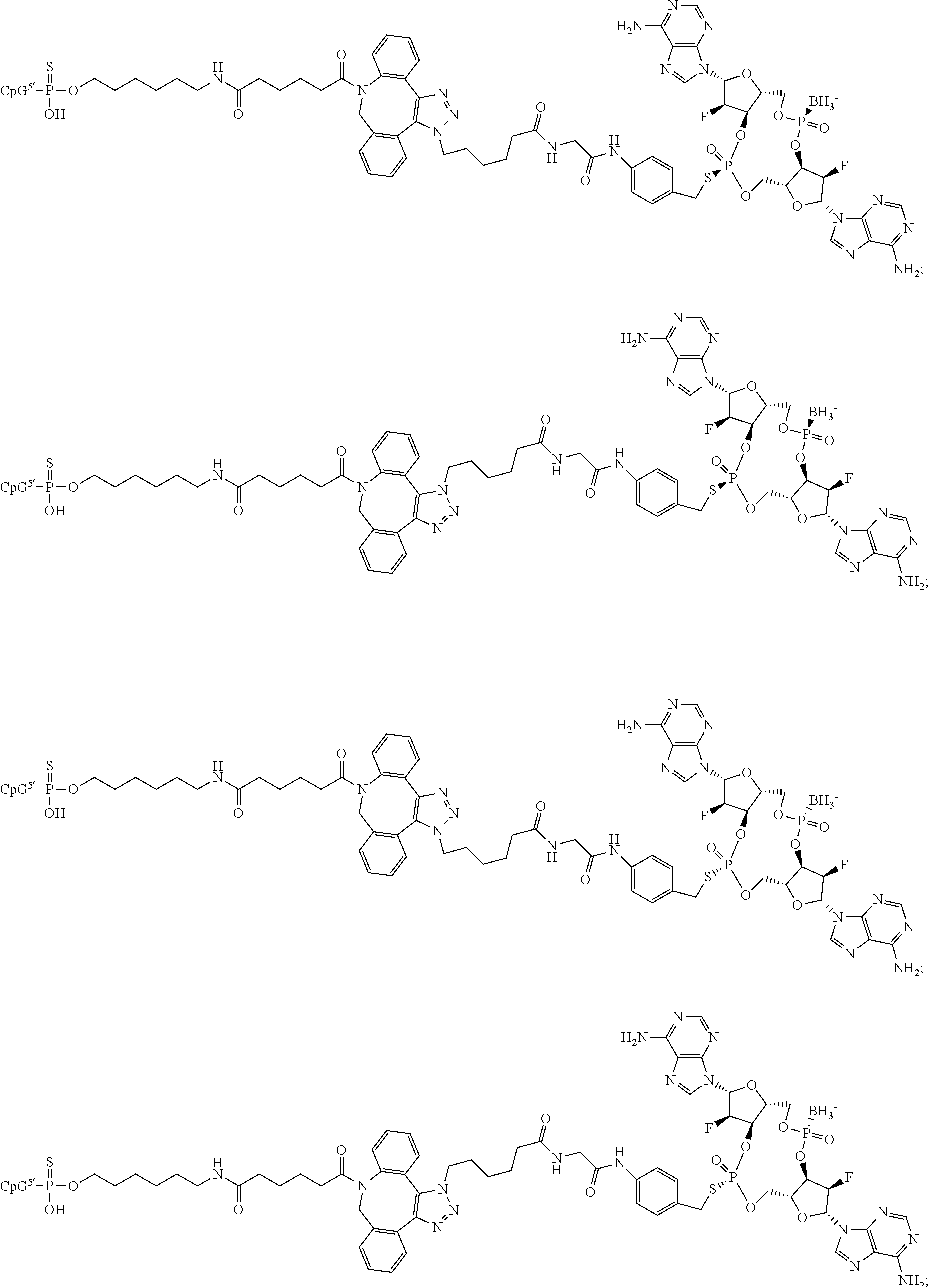

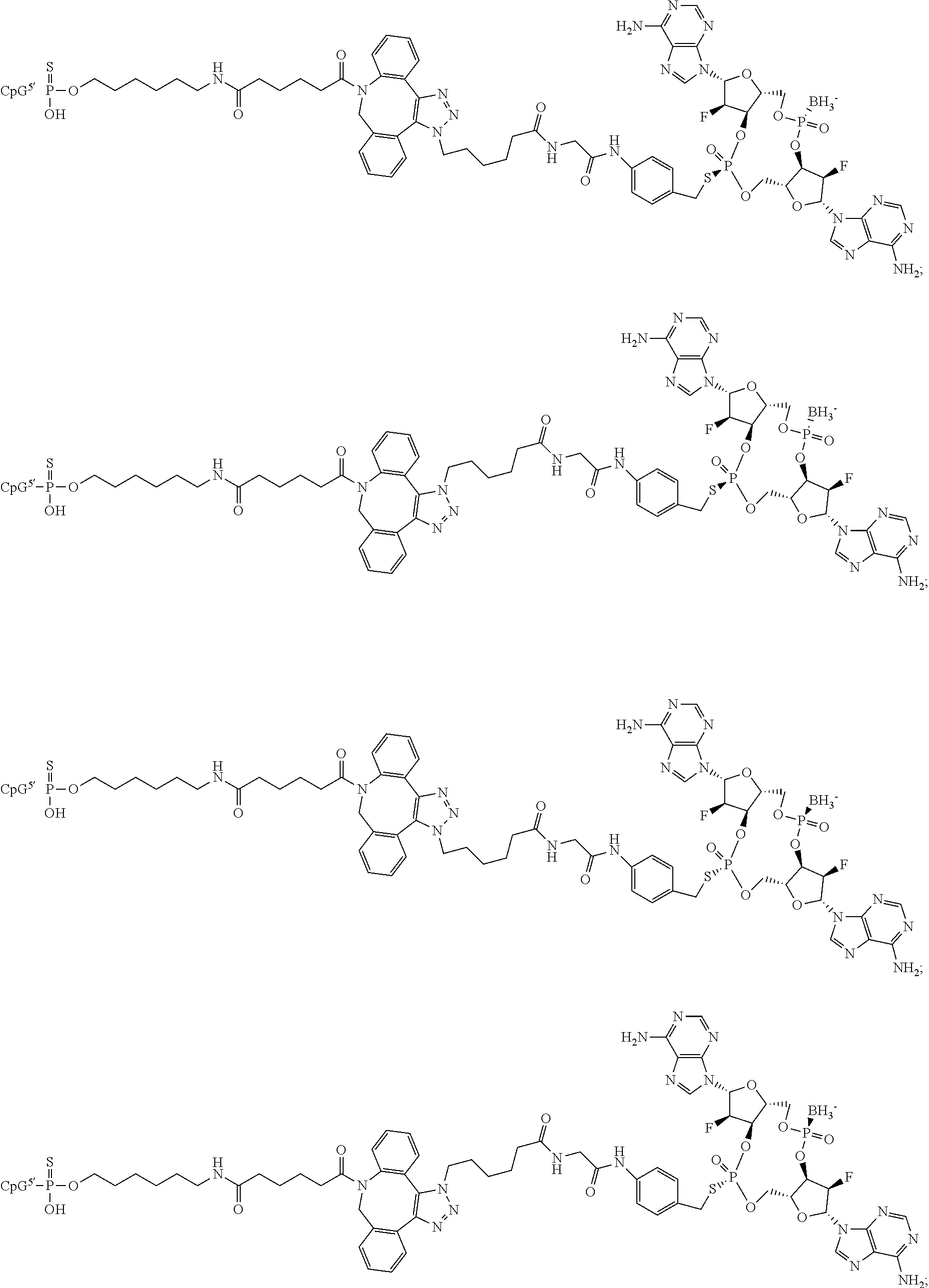

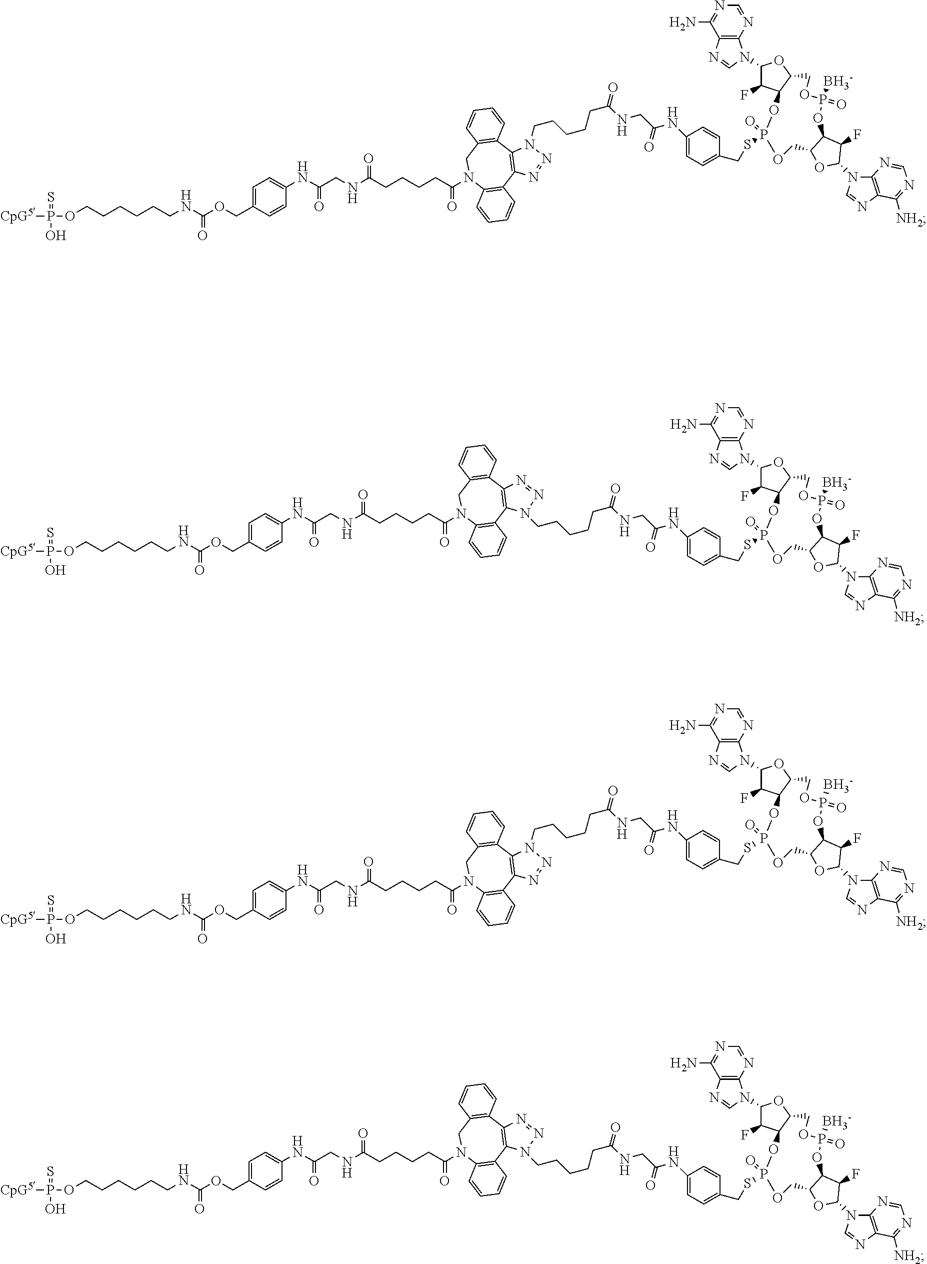

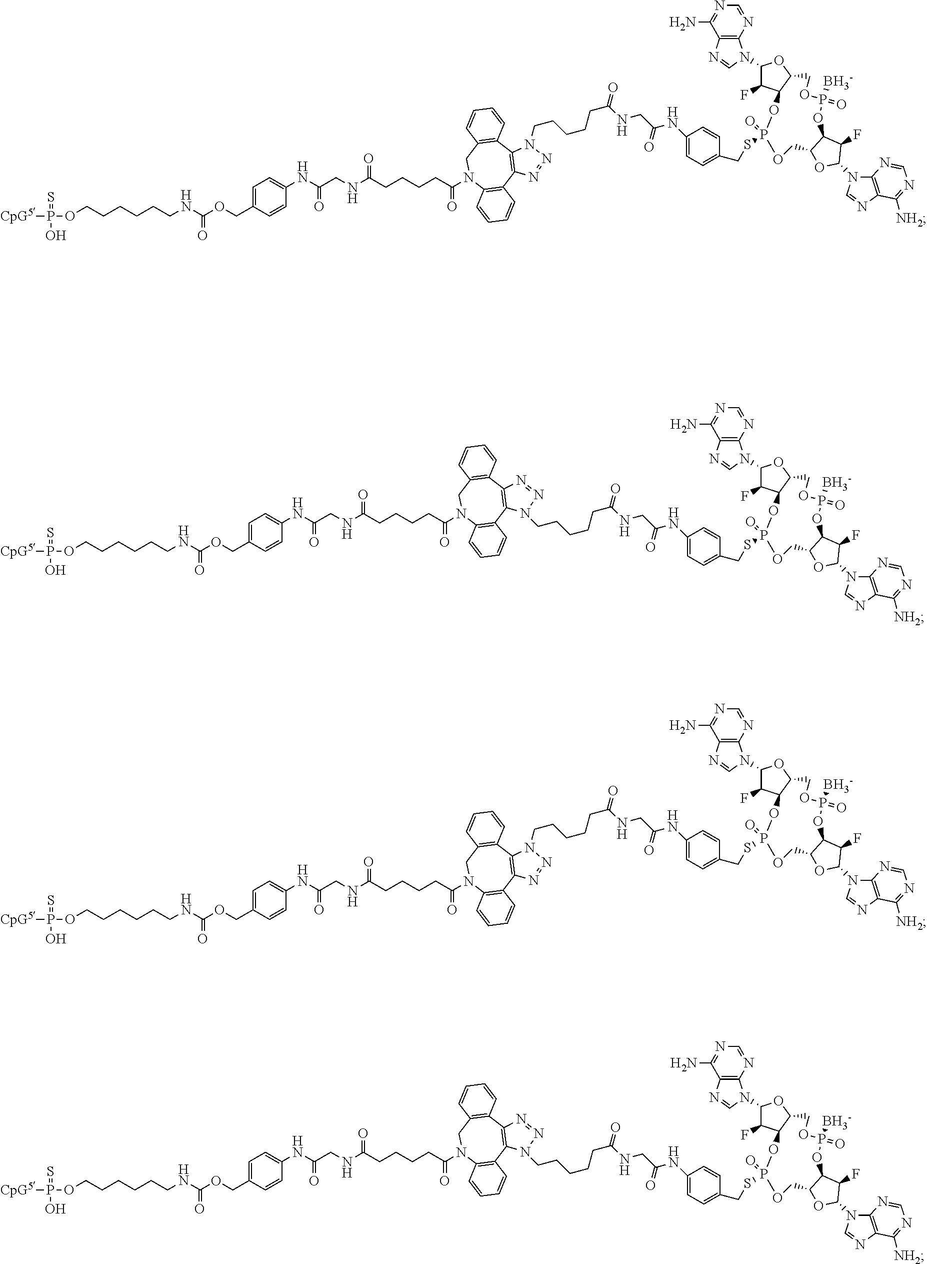

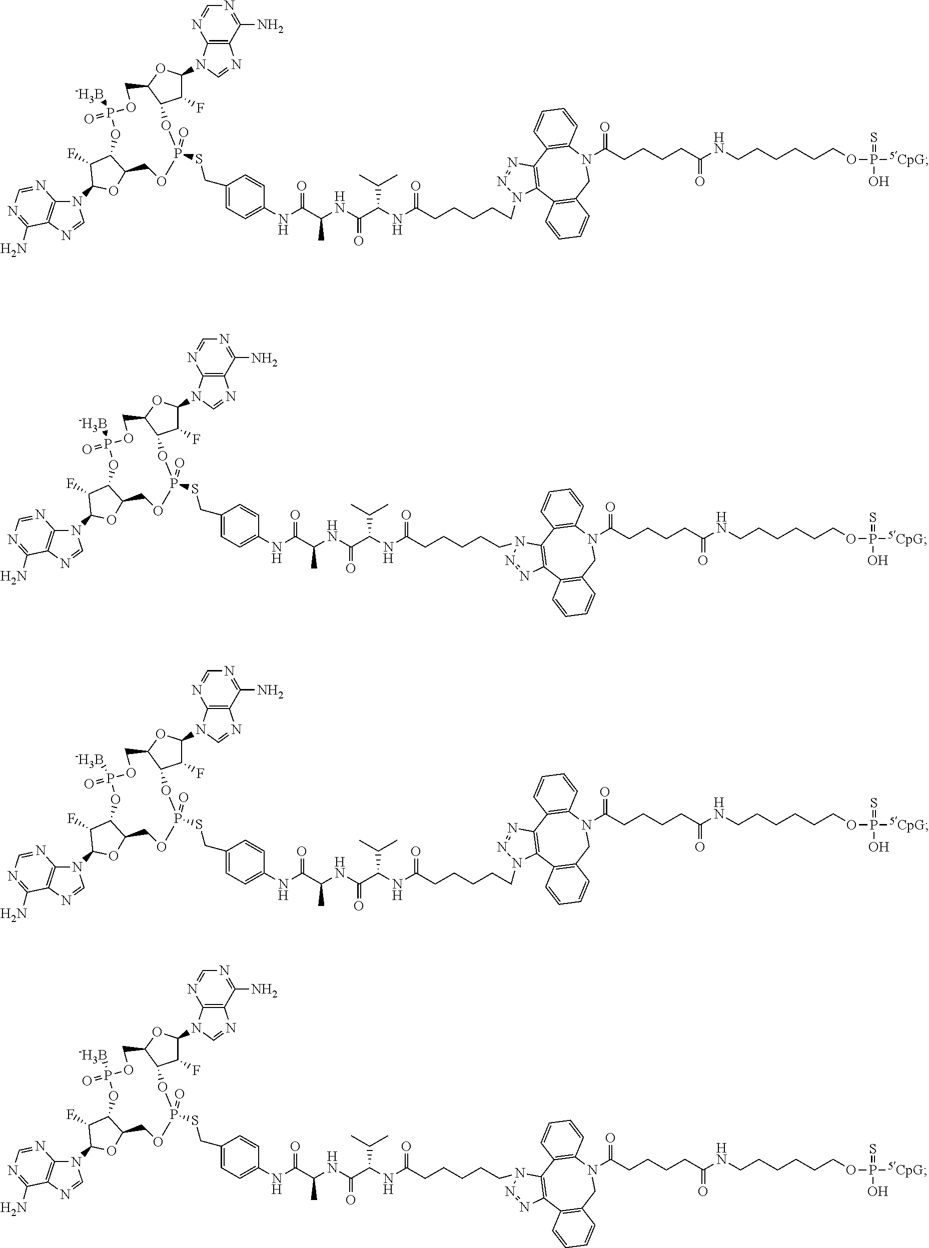

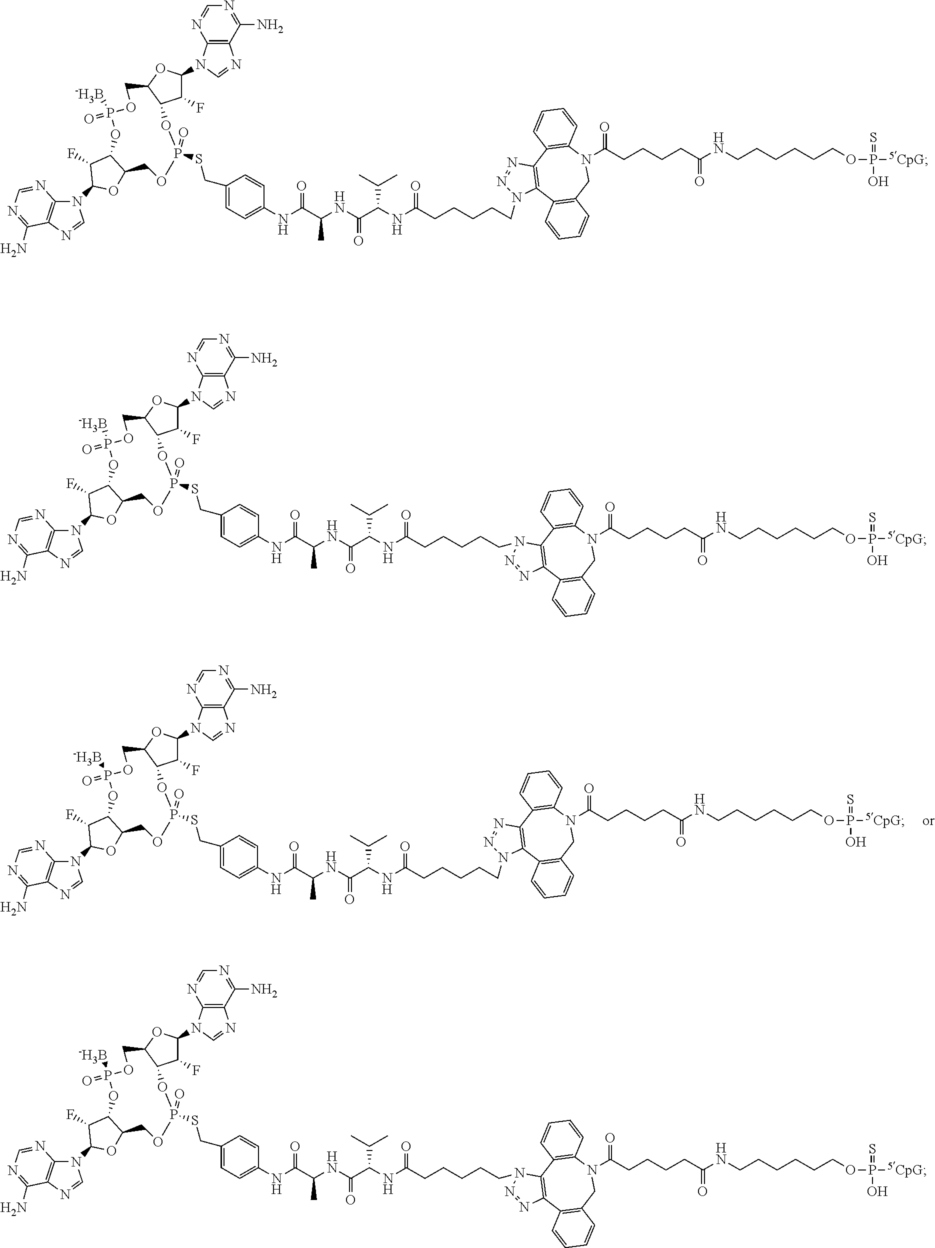

wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at 5′-O of the terminal nucleotide.

In certain embodiments, the present disclosure provides a conjugate of formula (XVI-1):

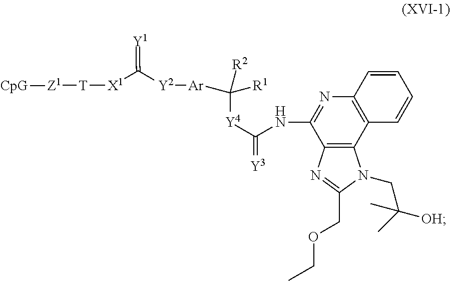

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
   wherein Z¹ and X¹ are each independently a spacer moiety;
   Ar is a substituted or unsubstituted aryl, or substituted or unsubstituted heteroaryl;
   R¹ and R² are each independently a hydrogen, substituted or unsubstituted alkyl, substituted or unsubstituted alkenyl, substituted or unsubstituted alkynyl, substituted or unsubstituted aryl; or
   R¹ and R² together with the atom to which they are attached, can join together to form a 3-8 membered ring that can contain one or two heteroatoms;
   Y² is O, NH, or S;
   Y¹, Y³ and Y⁴ are each independently O or S; and
   CpG is a TLR9 agonist oligodeoxynucleotide.

In certain embodiments, the present disclosure provides a conjugate has a structure according to formula (XVI):

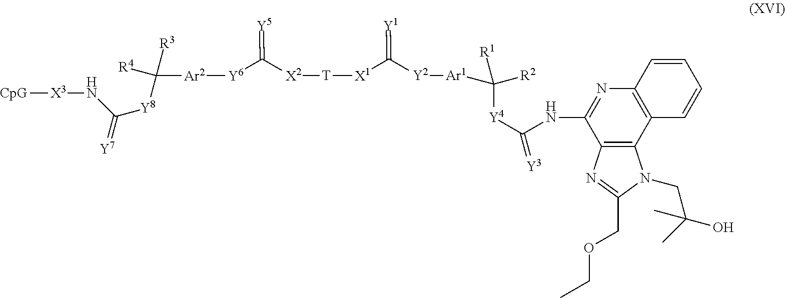

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
Ar¹ and Ar² are each independently a substituted or unsubstituted aryl or heteroaryl;
X¹, X² and X³ are each independently a spacer moiety;
R¹, R², R³ and R⁴ are each independently a hydrogen, alkyl, substituted alkyl, substituted or unsubstituted alkenyl, substituted or unsubstituted alkynyl, aryl, or substituted aryl; or
R¹ and R² together with the atom to which they are attached, can join together to form a 3-8 membered ring that can contain one or two heteroatoms; or
R³ and R⁴ together with the atom to which they are attached, can join together to form a 3-8 membered ring that can contain one or two heteroatoms;
Y¹, Y², Y³, Y⁴, Y⁵, Y⁶, Y⁷ and Y⁸ are each independently O or S;
T is a triazole functional group; and
CpG is a TLR9 agonist oligodeoxynucleotide.

In certain embodiments, the conjugate of formula (XVI) has a structure according to formula (XVI-A):

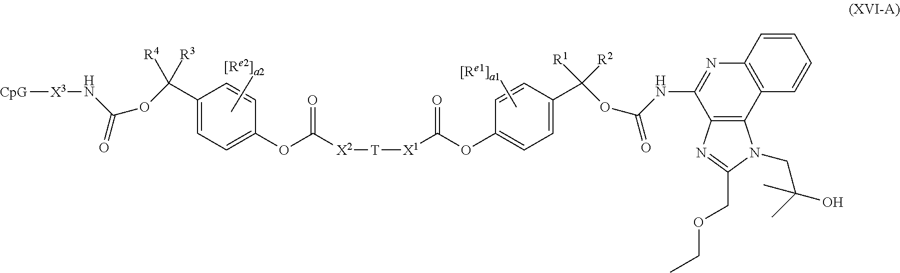

wherein:
X¹ and X² are each independently selected from spacers C1 to C17 as described herein; wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group.
X³ is selected from **—C₃-C₁₂ alkylene-L¹-*; and spacers B1 to B5 as described herein: wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
R¹, R², R³ and R⁴ are each independently a hydrogen, Me, or Et;
a1 and a2 are each independently an integer of 0 to 2;
R^{e1} and R^{e2} are each independently selected from nitro, cyano, halogen, —OMe, and —OCF₃;
T is a triazole functional group; and
CpG is a TLR9 agonist oligodeoxynucleotide.

In certain embodiments, the conjugate of formula (XVI) has a structure according to formula (XVI-B):

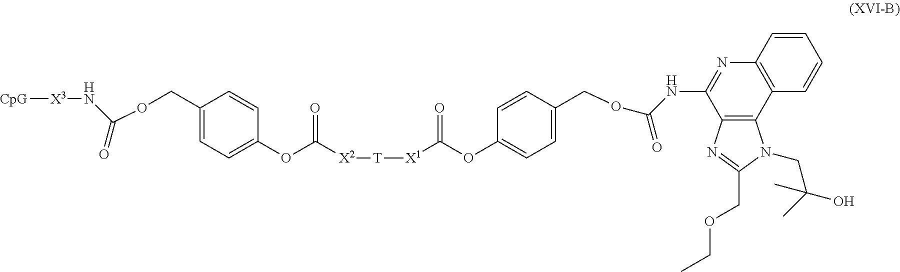

wherein:
X¹ and X² are each independently selected from spacers C1, C3, C8, C10, C13, C18 and C19 as described herein;
   wherein * indicates the attachment point connecting to T, ** indicates the attachment point connecting to the carbonyl group, and the spacer without * or ** indicates each of the two attachment points can be connected to either T or the carbonyl group;
X³ is selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
T is a triazole functional group; and
CpG is a TLR9 agonist oligodeoxynucleotide.

In certain embodiments of the conjugate of formula (XVI-B), wherein: X¹ is selected from spacers C1 and C3 as described herein; X² is selected from spacer C19 as described herein: wherein * indicates the attachment point connecting to T, ** indicates the attachment point connecting to the carbonyl group, and the spacer without * or ** indicates each of the two attachment points can be connected to either T or the carbonyl group; X³ is selected from spacer B3 as described herein: wherein L¹ is independently —OP(O)(OH)—, or —OP(S)(OH)—; and * indicates the attachment point connecting to a 3′-O of a terminal nucleotide of the CpG; and ** indicates the attachment point connecting to the amino group; and T is a triazole functional group. In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101), connecting at a 3′-O of a terminal nucleotide. In certain embodiments of the conjugate of formula (XVI-B), wherein: X¹ is C3.

In certain embodiments, the conjugate of formula (XVI) is represented by:

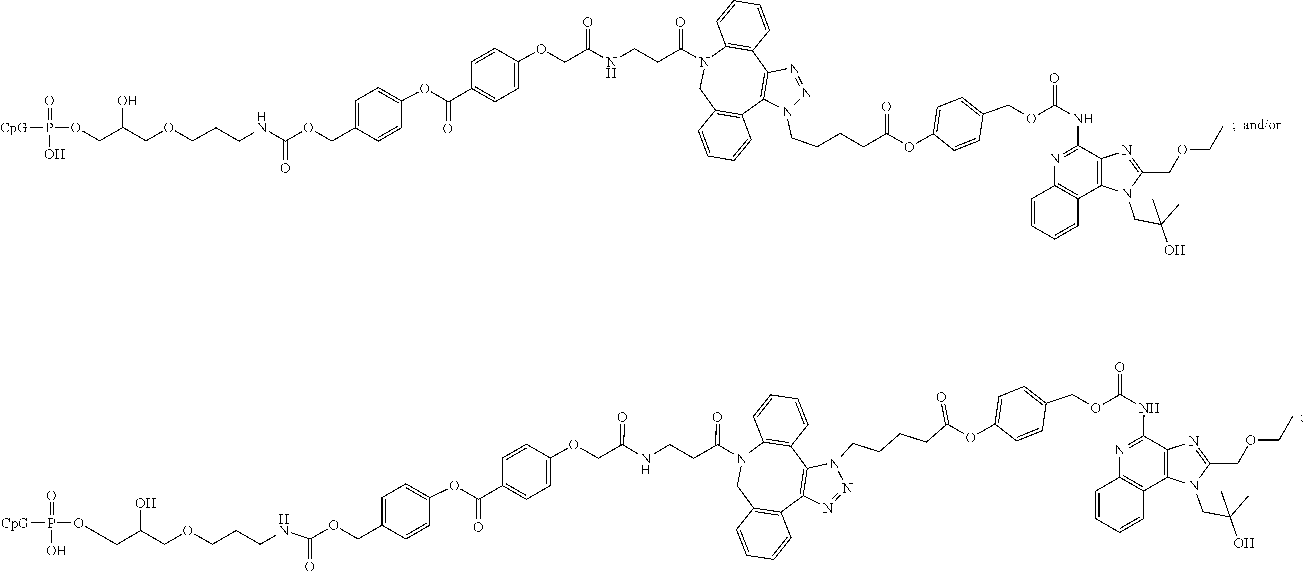

wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at a 3′-O of a terminal nucleotide.

In certain embodiments, the conjugate of formula (X) has a structure according to formula (XVII):

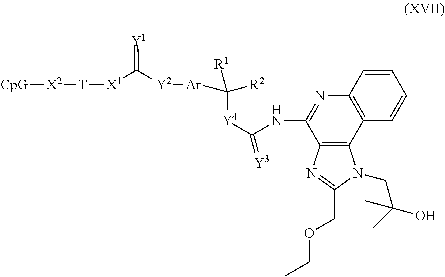

wherein:
Ar is a substituted or unsubstituted aryl, or substituted or unsubstituted heteroaryl;
X¹ and X² are each independently a spacer moiety;
R¹ and R² are each independently a hydrogen, alkyl, substituted alkyl, substituted or unsubstituted alkenyl, substituted or unsubstituted alkynyl, aryl, or substituted aryl; or
R¹ and R² together with the atom to which they are attached, can join together to form a 3-8 membered ring that can contain one or two heteroatoms;
Y¹, Y², Y³ and Y⁴ are each independently O or S;
T is a triazole functional group; and
CpG is a TLR9 agonist oligodeoxynucleotide.

In certain embodiments, the conjugate is a conjugate of formula (XVII-A):

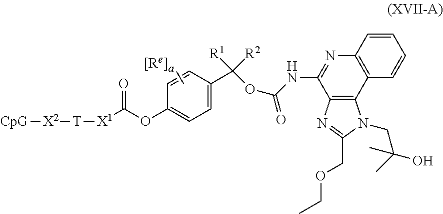

wherein:
X¹ is selected from spacers C1 to C17 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group.
X² is —X³—NH—CO—X⁴—, wherein X³ is selected **—C₃-C₁₂ alkylene-L¹-*; and spacers B1 to B5 as described herein: wherein L¹ is independently a bond, —OP(O)(OH)—, or —OP(S)(OH)—; and * indicates the attachment point connecting a 3′-O or 5′-O of a terminal nucleotide of the CpG; and ** indicates the attachment point connecting to the amino group;
X⁴ is selected from spacers C1, C3, C8, C10, C13, C18 and C19 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
R¹ and R² are each independently a hydrogen, Me, or Et;
T is a triazole functional group;
a is an integer of 0 to 2;
each R^{e} is independently selected from nitro, cyano, halogen, —OMe, —NHMe, —NHAc, —NHSO₂Me, and —OCF₃; and
CpG is a TLR9 agonist oligodeoxynucleotide.

In certain embodiments, the conjugate of formula (XVII-A) has a structure according to formula (XVII-B):

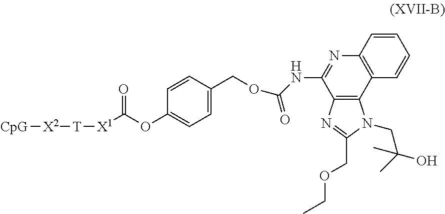

wherein:
X¹ is selected from spacers C1, C3, C8, C10, C13, C18 and C19 as described herein:
   wherein * indicates the attachment point connecting to T, ** indicates the attachment point connecting to the carbonyl group, and the spacer without * or ** indicates each of the two attachment points can be connected to either T or the carbonyl group;
X² is —X³—NH—CO—X⁴—, wherein X³ is selected from **—C₃-C₁₂ alkylene-L¹-*; and spacers B1 to B5 as described herein: wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
X⁴ is selected from spacers C1, C3, C8, C10, C13, C18 and C19 as described herein:
   wherein * indicates the attachment point connecting to T, ** indicates the attachment point connecting to the carbonyl group, and the spacer without * or ** indicates each of the two attachment points can be connected to either T or the carbonyl group;
T is a triazole functional group; and
CpG is a TLR9 agonist oligodeoxynucleotide.

In certain embodiments of the conjugate of formula (XVII-B), X¹ is selected from spacers C1, C3 and C13 as described herein: wherein * indicates the attachment point connecting to T, ** indicates the attachment point connecting to the carbonyl group, and the spacer without * or ** indicates each of the two attachment points can be connected to either T or the carbonyl group; X² is —X³—NH—CO—X⁴—, wherein X³ is spacer B3 as described herein: wherein L¹ is independently —OP(O)(OH)—, or —OP(S)(OH)—; * indicates the attachment point connecting to a 3′-O of a terminal nucleotide of the CpG; and ** indicates the attachment point connecting to the amino group; X⁴ is selected from spacers C1, C3 and C13 as described herein: wherein * indicates the attachment point connecting to T, ** indicates the attachment point connecting to the carbonyl group, and the spacer without * or ** indicates each of the two attachment points can be connected to either T or the carbonyl group; T is a triazole functional group. In certain embodiments, CpG is a phosphorothioate linked 5′-T*C*G *A*A*C *G*T*T*C*G*A *A*C*G *T*T*C *G*A*A *C*G*T *T*C*G *A*A*T-3′ (SD-101), connecting at 3′-O of the terminal nucleotide.

In certain embodiments of the conjugate of formula (XVII-B), X¹ is selected from spacers C3 and C13 as described herein, and X⁴ is selected from spacers C3 and C13 as described herein.

In certain embodiments, the conjugate is represented by:

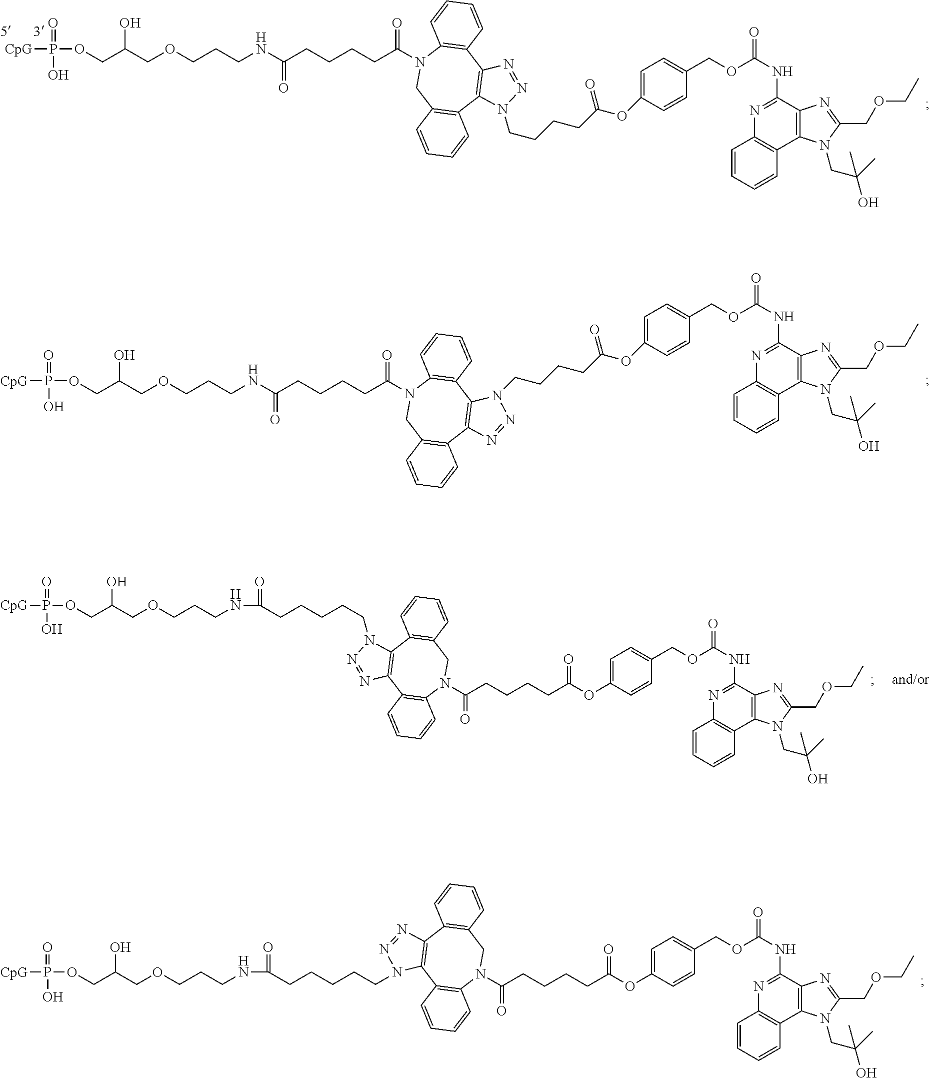

wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide.

In certain embodiments, the conjugate of formula (X) has a structure according to formula (XVIII):

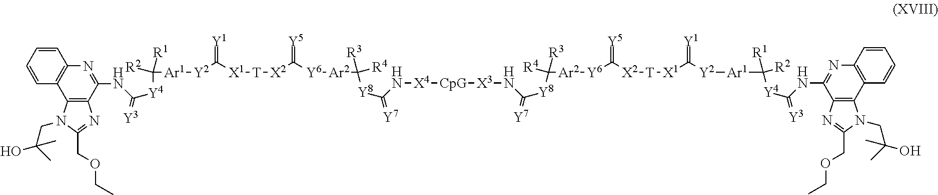

wherein:
Ar¹ and Ar² are each independently a substituted or unsubstituted aryl, or substituted or substituted heteroaryl;
X¹, X², X³ and X⁴ are each independently a spacer moiety;
R¹, R², R³ and R⁴ are each independently a hydrogen, alkyl, substituted alkyl, substituted or unsubstituted alkenyl, substituted or unsubstituted alkynyl, aryl, or substituted aryl; or
R¹ and R² together with the atom to which they are attached, can join together to form a 3-8 membered ring that can optionally contain one or two heteroatoms; or
R³ and R⁴ together with the atom to which they are attached, can join together to form a 3-8 membered ring that can optionally contain one or two heteroatoms;
Y² and Y 6 are each independently O, NH or S;
Y¹, Y³, Y⁴, Y⁵, Y⁷ and Y⁸ are each independently O or S;
   each T is independently a triazole functional group; and
   CpG is a TLR9 agonist oligodeoxynucleotide.

In certain embodiments, the conjugate of formula (X) has a structure according to formula (XVIII-A):

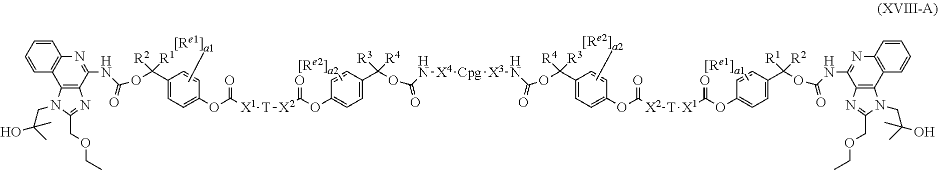

wherein:
X¹ and X² are each independently selected from spacers C1 to C17 as described herein;
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group.
X³ and X⁴ are each independently selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
R¹, R², R³ and R⁴ are each independently a hydrogen, Me, or Et;
each T is independently a triazole functional group;
a1 and a2 are each independently an integer of 0 to 2;
each R^{e1} and R^{e2} are independently selected from nitro, cyano, halogen, —OMe, and —OCF₃; and
CpG is a TLR9 agonist oligodeoxynucleotide.

In certain embodiments, the conjugate of formula (X) has formula (XVIII-B):

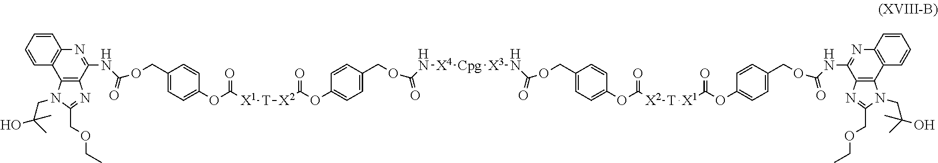

wherein:
X¹ and X² are each independently selected from spacers C1, C3, C8, C10, C13, C18 and C19 as described herein;
   wherein * indicates the attachment point connecting to T, ** indicates the attachment point connecting to the carbonyl group, and the spacer without * or ** indicates each of the two attachment points can be connected to either T or the carbonyl group;
X³ and X⁴ are each independently selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
each T is independently a triazole functional group; and
CpG is a TLR9 agonist oligodeoxynucleotide.

In certain embodiments of the conjugate of formula (XVIII-B), X¹ is selected from spacers C1 and C3 as described herein and X² is selected from spacer C19 as described herein: wherein * indicates the attachment point connecting to T, ** indicates the attachment point connecting to the carbonyl group, and the spacer without * or ** indicates each of the two attachment points can be connected to either T or the carbonyl group; X⁴ is **—C₃-C₁₂ alkylene-L¹-*; wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; and X³ is spacer B3 as described herein: wherein L¹ is independently —OP(O)(OH)—, or —OP(S)(OH)—; and * indicates the attachment point connecting to a 3′-O of a terminal nucleotide of the CpG; and ** indicates the attachment point connecting to the amino group; each T is independently a triazole functional group. In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101), wherein X⁴ is covalently bound to a 5′-O of a terminal nucleotide of CpG and X³ is covalently bound to a 3′-O of a terminal nucleotide of CpG.

In certain embodiments, the conjugate of formula (X) has a structure according to formula (XIX):

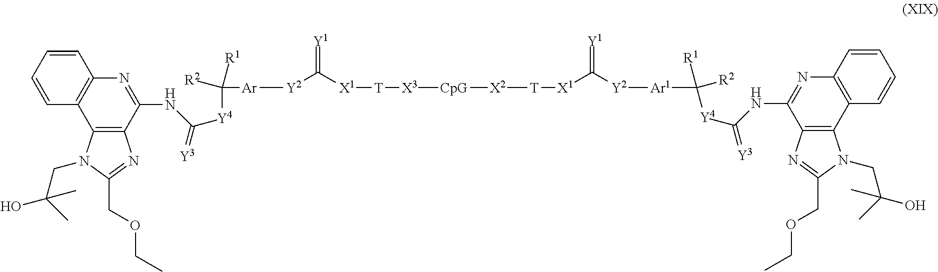

wherein:
Ar is a substituted or unsubstituted aryl, or substituted or unsubstituted heteroaryl;
X¹, X² and X³ are each independently a spacer moiety;
R¹ and R² are each independently a hydrogen, alkyl, substituted alkyl, substituted or unsubstituted alkenyl, substituted or unsubstituted alkynyl, aryl, or substituted aryl; or
R¹ and R² together with the atom to which they are attached, can join together to form a 3-8 membered ring that can contain one or two heteroatoms;
Y² is O, NH or S;
Y¹, Y³ and Y⁴ are each independently O or S;
each T is independently a triazole functional group; and
CpG is a TLR9 agonist oligodeoxynucleotide.

In certain embodiments, the conjugate of formula (X) has a structure according to formula (XIX-A):

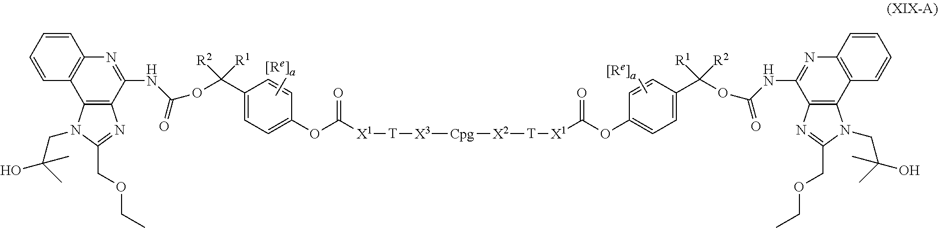

wherein:
X¹ is selected from spacers C1 to C17 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group.
X² and X³ are each independently —X⁴—NH—CO—X⁵—, wherein X⁴ is selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
X⁵ is selected from spacers C1, C3, C8, C10, C13, C18 and C19 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates each of the two attachment points can be connected to either T or the carbonyl group;
R¹ and R² are each independently a hydrogen, Me, or Et;
each T is independently a triazole functional group;
a is an integer of 0 to 2;
each R^{e} is independently selected from nitro, cyano, halogen, —OMe, —NHMe, —NHAc, —NHSO₂Me, and —OCF₃; and
CpG is a TLR9 agonist oligodeoxynucleotide.

In certain embodiments, the conjugate of formula (X) has a structure according to formula (XIX-B):

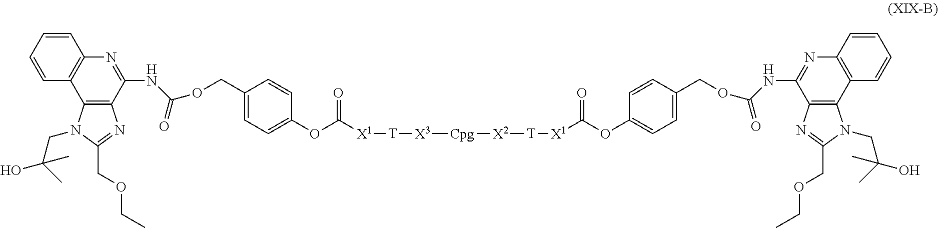

wherein:
X¹ is selected from spacers C1, C3, C8, C10, C13, C18 and C19 as described herein:
   wherein * indicates the attachment point connecting to T, ** indicates the attachment point connecting to the carbonyl group, and the spacer without * or ** indicates each of the two attachment points can be connected to either T or the carbonyl group;
X² and X³ are each independently —X⁴—NH—CO—X⁵—, wherein X⁴ is selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
X⁵ is selected from spacers C1, C3, C8, C10, C13, C18 and C19 as described herein:
   wherein * indicates the attachment point connecting to T, ** indicates the attachment point connecting to the carbonyl group, and the spacer without * or ** indicates each of the two attachment points can be connected to either T or the carbonyl group;
each T is independently a triazole functional group; and
CpG is a TLR9 agonist oligodeoxynucleotide.

In certain embodiments of the conjugate of formula (XIX-B), X¹ is selected from spacers C1 and C3 as described herein: wherein * indicates the attachment point connecting to T, ** indicates the attachment point connecting to the carbonyl group, and the spacer without * or ** indicates each of the two attachment points can be connected to either T or the carbonyl group; X² and X³ are each independently —X⁴—NH—CO—X⁵—, wherein X⁴ is **—C₃-C₁₂ alkylene-L¹-*; wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; or X⁴ is spacer B3 as described herein: wherein L¹ is independently —OP(O)(OH)—, or —OP(S)(OH)—; and * indicates the attachment point connecting to a 3′-O of a terminal nucleotide of the CpG; and ** indicates the attachment point connecting to the amino group; X⁵ is selected from spacer C13 as described herein: wherein * indicates the attachment point connecting to T, and ** indicates the attachment point connecting to the carbonyl group; each T is independently a triazole functional group. In certain embodiments, CpG is a phosphorothioate linked 5′-T*C*G *A*A*C *G*T*T *C*G*A *A*C*G *T*T*C*G*A*A *C*G*T *T*C*G *A*A*T-3′ (SD-101).

In certain embodiments, the conjugate of formula (X) has a structure according to formula (XXVIII):

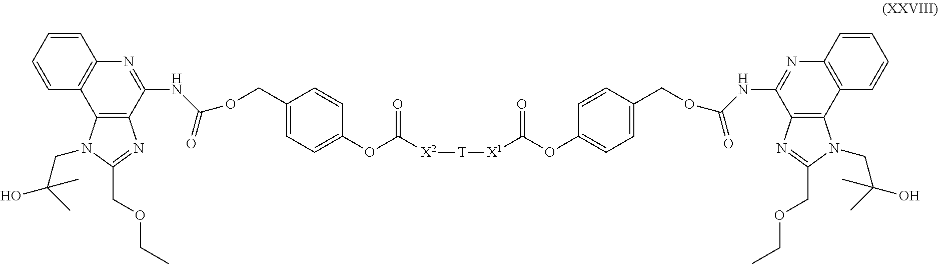

wherein:
X¹ and X² are each independently selected from spacer C1, C3, and C13 as described herein;
wherein * indicates the attachment point connecting to T, ** indicates the attachment point connecting to the carbonyl group, and the spacer without * or ** indicates each of the two attachment points can be connected to either T or the carbonyl group; and
T is a triazole functional group.

In certain embodiments of the conjugate of formula (XXVIII), X¹ and X² are each independently selected from spacer C3, and C13 as described herein.

In certain embodiments, the conjugate is represented by:

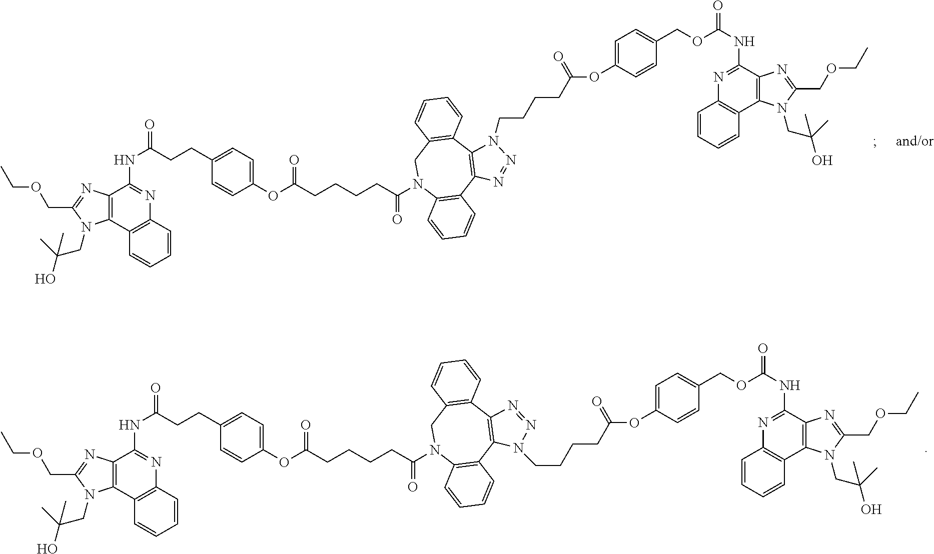

In certain embodiments, the present disclosure provides a STING agonist that is released from the conjugates of formulae (XXIV), (XXVI), (XXVI-A) and (XXVI-B), wherein the released STING agonist has a structure according to formula (XXVIV):

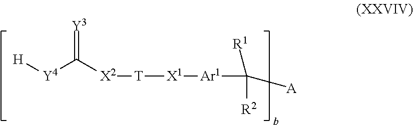

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
Ar¹ is a substituted or unsubstituted aryl, or substituted or unsubstituted heteroaryl;
X¹ and X² are each independently a spacer moiety;
R¹ and R² are each independently a hydrogen, alkyl, substituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
Y³ and Y⁴ are each independently O or S;
b is an integer of 1 or 2; wherein when b=2, both groups are directly bound to A; each T is independently a triazole functional group; and
A is a STING agonist.
   In certain embodiments of the STING agonist of formula (XXVIV), wherein the STING agonist is selected from: ADU-S100, MK-1454, BMS-986301, GSK3745417, E7766, SB 11285,

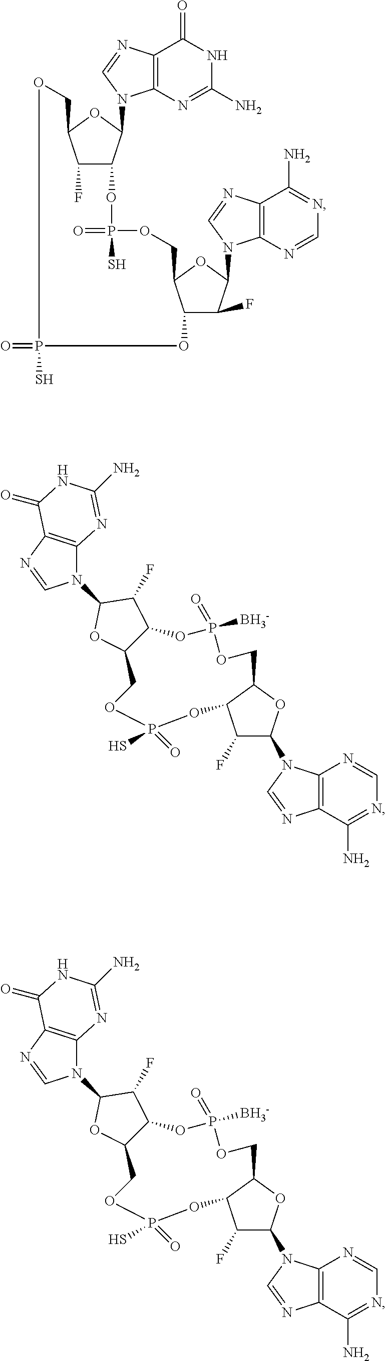

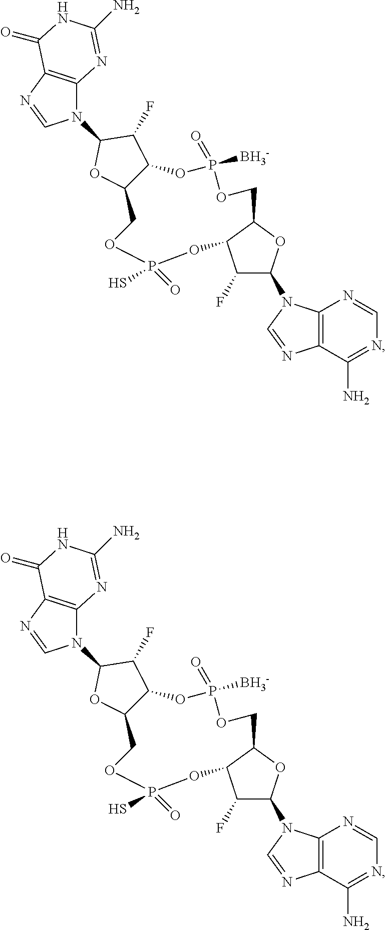

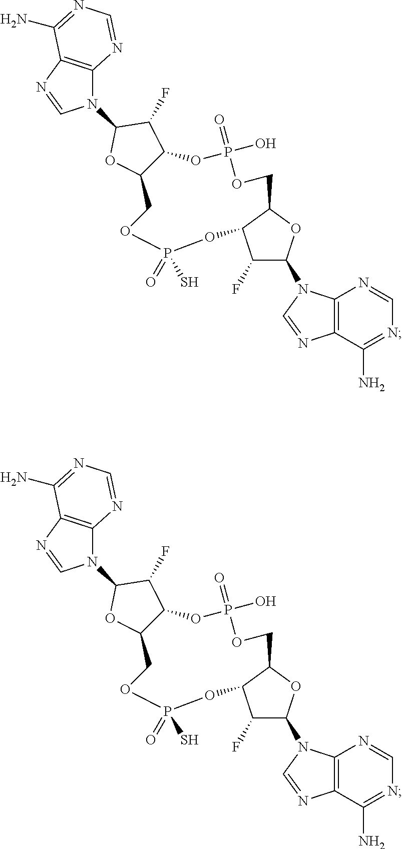 [SMILES: Nc1ncnc2c1ncn2[C@@H]1O[C@@H]2CO[P@@](=O)(S)O[C@H]3[C@@H](F)[C@H](n4cnc5c(N)ncnc54)O[C@@H]3COP(=O)(O)O[C@H]2[C@H]1F ; Nc1ncnc2c1ncn2[C@@H]1O[C@@H]2CO[P@](=O)(S)O[C@H]3[C@@H](F)[C@H](n4cnc5c(N)ncnc54)O[C@@H]3COP(=O)(O)O[C@H]2[C@H]1F]

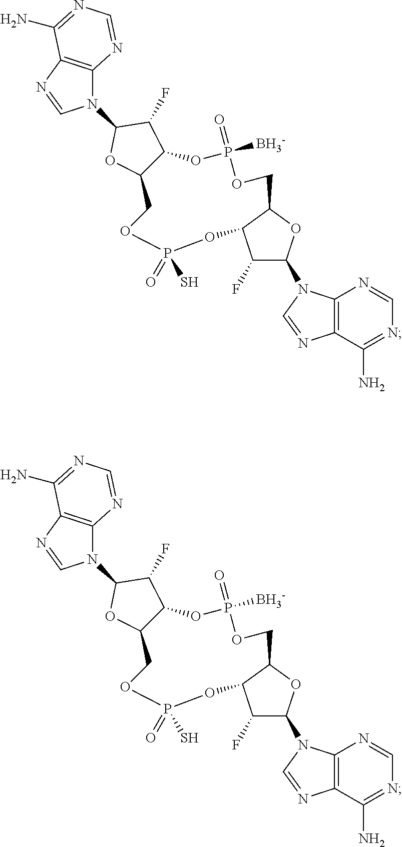

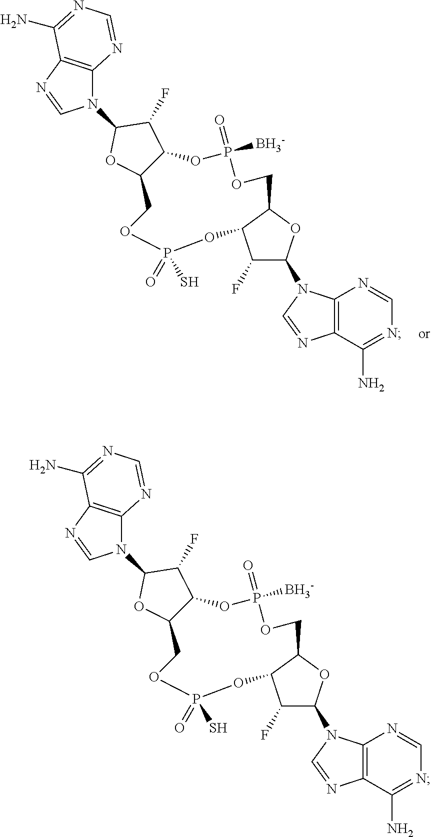

or a stereoisomer thereof.

In certain embodiments, the released STING agonist has a structure according to formula (XXVIV-A):

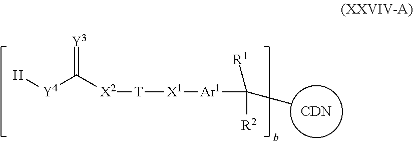

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
Ar¹ is a substituted or unsubstituted aryl, or substituted or unsubstituted heteroaryl;
X¹ and X² are each independently a spacer moiety;
R¹ and R² are each independently a hydrogen, alkyl, substituted alkyl, substituted or unsubstituted alkenyl, substituted or unsubstituted alkynyl, aryl, or substituted aryl; or
R¹ and R², together with the atom to which they are attached, can join together to form a 3-8 membered ring that can optionally contain one or two heteroatoms;
Y³ and Y⁴ are each independently O or S;
b is an integer of 1 or 2; wherein when b=2, both groups are directly bound to CDN;
each T is independently a triazole functional group;
CDN is a cyclic dinucleotide that is a STING agonist; and
the linker is covalently bound to a thiol group of the cyclic dinucleotide STING agonist.

In certain embodiments, the released STING agonist has a structure according to formula (XXVIV-B):

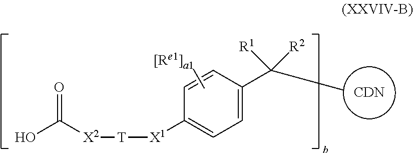

wherein:
X¹ is selected from spacers D1 to D12 as described herein:
   wherein * indicates the attachment point connecting to selected T, ** indicates the attachment point connecting to the phenyl group, and the spacer without * or ** indicates the attachment points can be connected to either T or the phenyl group;
X² is selected from spacers C13 to C17 as described herein;
   wherein oxygen can be optionally replaced by NH, NMe, NAc, S, or SO₂; * indicates the attachment point connecting to T, and ** indicates the attachment point connecting to the carbonyl group;
R¹ and R² are each independently a hydrogen, Me, or Et;
each T is independently a triazole functional group;
a1 is an integer of 0 to 2;
b is an integer of 1 or 2; wherein when b=2, both groups are directly bound to CDN;
R^{e1} is selected from nitro, cyano, halogen, —OMe, and —OCF₃;
CDN is a cyclic dinucleotide that is a STING agonist; and
the linker is covalently bound to a thiol group of the cyclic dinucleotide STING agonist.

In certain embodiments, the released STING agonist has a structure according to formula (XXVIV-C):

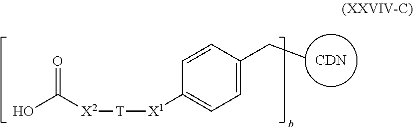

wherein:
X¹ is selected from spacer D5 and D6 as described herein: wherein * indicates the attachment point connecting to selected T, and ** indicates the attachment point connecting to the phenyl group;
X² is selected from spacer C19 as described herein: wherein * indicates the attachment point connecting to T, and ** indicates the attachment point connecting to the carbonyl group;
b is an integer of 1 or 2; wherein when b=2, both groups are directly bound to CDN;
each T is independently a triazole functional group;
CDN is a cyclic dinucleotide that is a STING agonist; and
the linker is covalently bound to a thiol group of the cyclic dinucleotide STING agonist.

In certain embodiments of released STING agonists of formulae (XXVIV), (XXVIV-A), (XXVIV-B) and (XXVIV-C), b is one. In certain embodiments of released STING agonists of formulae (XXVIV), (XXVIV-A), (XXVIV-B) and (XXVIV-C), b is two.

In certain embodiments of released STING agonists of formulae (XXVIV), (XXVIV-A), (XXVIV-B) and (XXVIV-C), wherein b is one and the cyclic dinucleotide is:

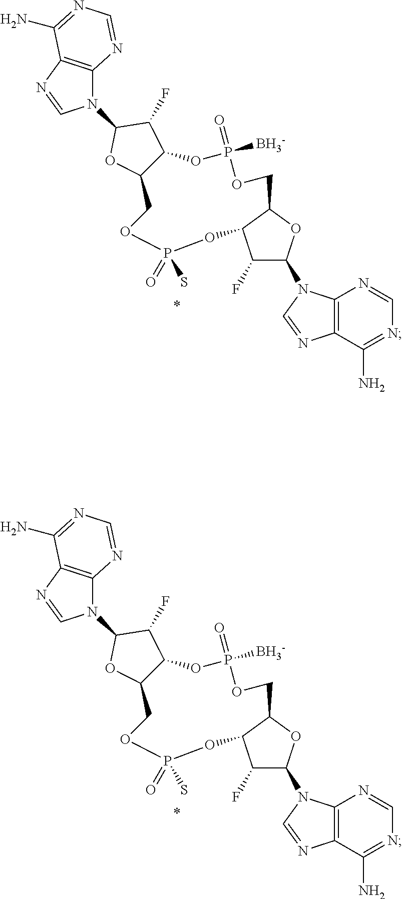

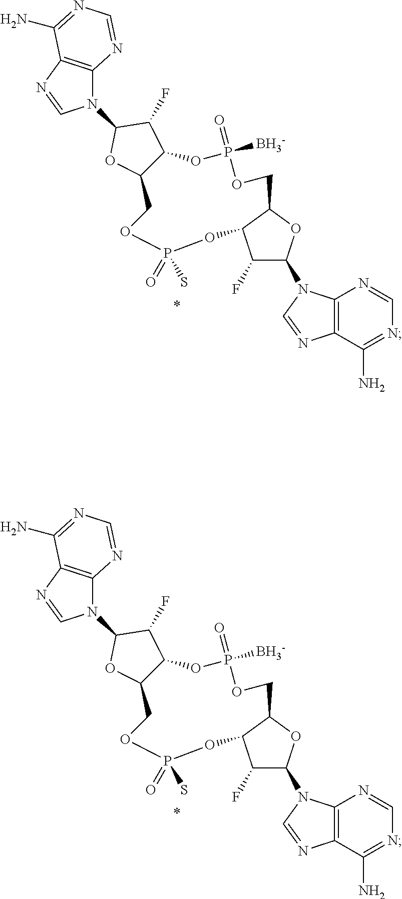

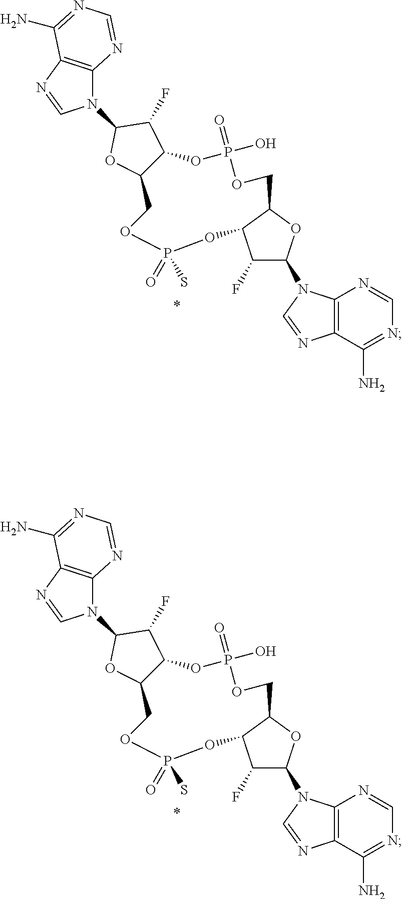 [SMILES: Nc1ncnc2c1ncn2[C@@H]1O[C@@H]2CO[P@@](=O)(S)O[C@H]3[C@@H](F)[C@H](n4cnc5c(N)ncnc54)O[C@@H]3COP(=O)(O)O[C@H]2[C@H]1F ; Nc1ncnc2c1ncn2[C@@H]1O[C@@H]2CO[P@](=O)(S)O[C@H]3[C@@H](F)[C@H](n4cnc5c(N)ncnc54)O[C@@H]3COP(=O)(O)O[C@H]2[C@H]1F]

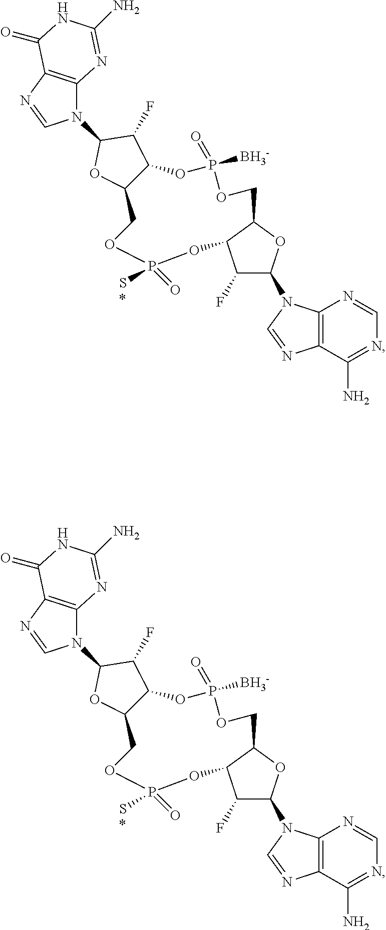

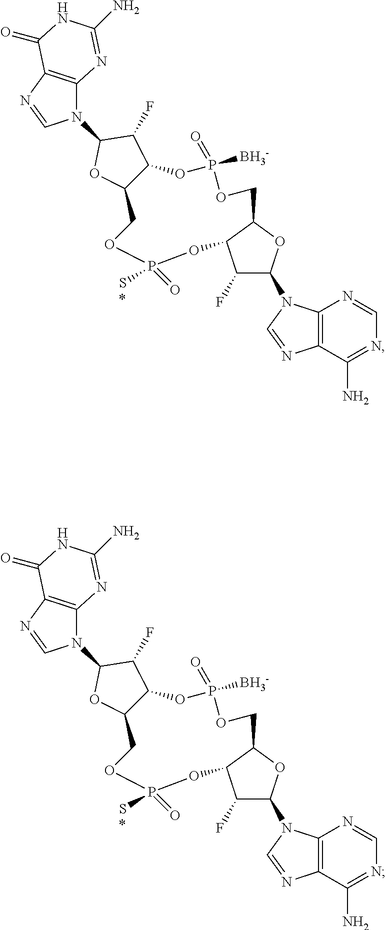

or a stereoisomer thereof; wherein the * indicates the S of thiol group (—SH, H not shown where * appears) of the cyclic dinucleotide that is connecting with the linker.

In certain embodiments, the present disclosure provides a TLR9 agonist that is released from the conjugates of formulae (XII), (XII-A), (XII-B), (XVII), (XVII-A), and (XVII-B) wherein the released TLR9 agonist has a structure according to formula (XX):

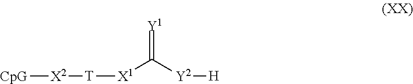

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
X¹ and X² are each independently a spacer moiety;
Y¹ and Y² are each independently O or S;
T is a triazole functional group; and
CpG is a TLR9 agonist oligodeoxynucleotide; wherein one or more atoms in the CpG is independently replaced with a covalent bond to X².

In certain embodiments, the disclosure provides conjugates of formulae (XIV), (XIV-A), (XIV-B), (XIX), (XIX-A), and (XIX-B) wherein the released TLR9 agonist has a structure according to formula (XXI):

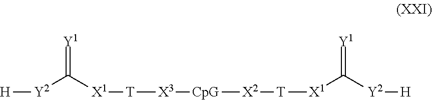

or an enantiomer, a mixture of enantiomers, a diastereomer, a mixture of two or more diastereomers, a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
X¹, X² and X³ are each independently a spacer moiety;
Y¹ and Y² are each independently O or S;
each T is independently a triazole functional group; and
CpG is a TLR9 agonist oligodeoxynucleotide; wherein one or more atoms in the CpG is independently replaced with a covalent bond to X² and X³.

In certain embodiments of the TLR9 agonists of formulae (XX) and (XXI), X¹ is selected from spacers C1 to C17 as described herein:
wherein each oxygen is independently and optionally replaced by NH, NMe, NAc, S, or SO₂; * indicates the attachment point connecting to T, ** indicates the attachment point connecting to the carbonyl or thiocarbonyl group, and the spacer without * or ** indicates the attachment points can be connected to either T or the carbonyl or thiocarbonyl group.
X² and X³ are each independently —X⁴—NH—CO—X⁵—, wherein X⁴ is selected from **—C₃-C₁₂ alkylene-L¹-*; and spacers B1 to B5 as described herein:
   wherein L¹ is independently a bond, —OP(O)(OH)—, or —OP(S)(OH)—; and * indicates the attachment point connecting to a 3′-O or 5′-O of a terminal nucleotide of the CpG; and ** indicates the attachment point connecting to the amino group;
   X⁵ is selected from spacers C1, C3, C8, C10, C13, C18 and C19 as described herein:
   wherein each oxygen is independently and optionally replaced by NH, NMe, NAc, S, or SO₂; * indicates the attachment point connecting to T, ** indicates the attachment point connecting to the carbonyl group, and the spacer without * or ** indicates the attachment points can be connected to either T or the carbonyl group; and
Y¹ and Y² are O.

In certain embodiments of the TLR9 agonists of formulae (XX) and (XXI), XI is selected from spacers C1, C3, C8, C10 to C19 as described herein: wherein * indicates the attachment point connecting to T, ** indicates the attachment point connecting to the carbonyl group, and the spacer without * or ** indicates the attachment points can be connected to either T or the carbonyl group.
X² and X³ are each independently —X⁴—NH—CO—X⁵—, wherein X⁴ is **—C₃-C₁₂ alkylene-L¹-*; wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; or X⁴ is spacer B3 as described herein: wherein L¹ is independently —OP(O)(OH)—, or —OP(S)(OH)—; and * indicates the attachment point connecting to a 3′-O of a terminal nucleotide of the CpG; ** indicates the attachment point connecting to the amino group;
   X⁵ is selected from spacers C1, C3 and C13 as described herein: wherein * indicates the attachment point connecting to T, ** indicates the attachment point connecting to the carbonyl group, and the spacer without * or ** indicates the attachment points can be connected to either T or the carbonyl group;
Y¹ and Y² are O.

In certain embodiments of the TLR9 agonists of formulae (XX) and (XXI), wherein X¹ is selected from spacers C3, C10 to C19 as described herein, and X⁵ is selected from spacers C3 and C13 as described herein.

In certain embodiments, CpG is a TLR9 agonists CpG ODNs (oligodeoxynucleotides) that is short synthetic single-stranded DNA molecules containing unmethylated CpG motifs in following sequences: a phosphorothioate linked 5′-T*C*G *A*A*C *G*T*T *C*G*A *A*C*G *T*T*C *G*A*A *C*G*T *T*C*G *A*A*T-3′ (SD-101), connecting at 3′-O or 5′-O of the terminal nucleotide.

In certain embodiments of the TLR9 agonists of formulae (XX), the TLR9 agonist has following structure:

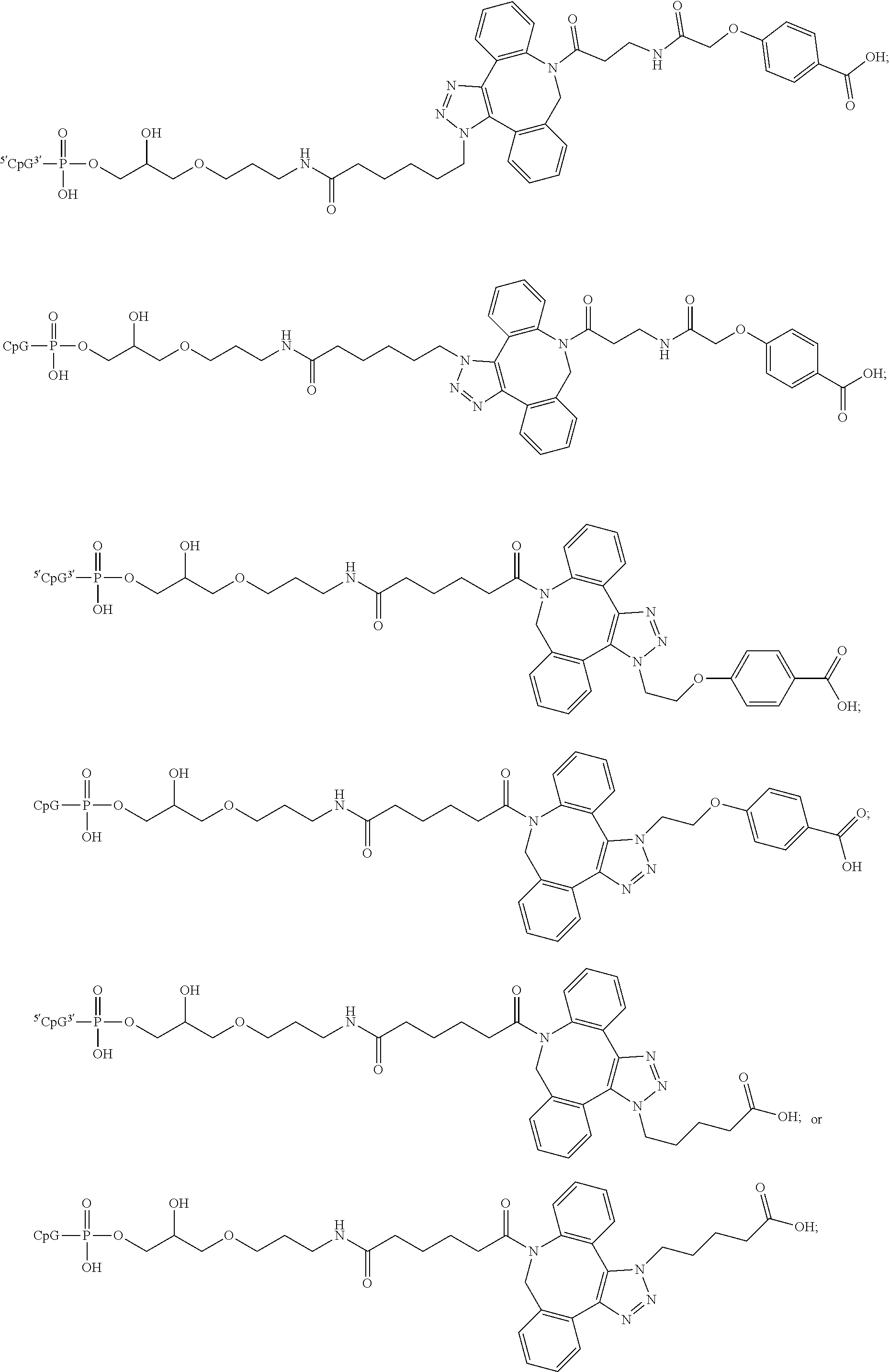

wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ connecting at 3′-O of the terminal nucleotide; or
the TLR9 agonist has following structure

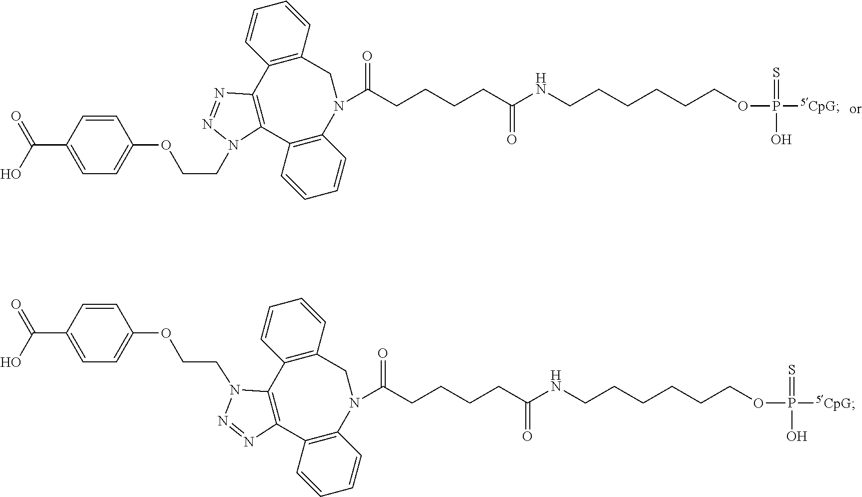

wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ connecting at 5′-O of the terminal nucleotide.

In certain embodiments of the compounds of formulae (I), (V), (V-A), (V-B), (VI), (VII), (VII-A), (VII-B), (VIII), (X-1), (XI), (XI-A), (XI-B), (XII), (XII-A), (XII-B), (XIII), (XIII-A), (XIII-B), (XIV), (XIV-A), (XIV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A) and (XXVII-B), wherein CpG comprises a following formula:
5′X₁X₂CGX₃X₄3′
wherein X₁, X₂, X₃, and X₄ are any nucleotide, and
CpG is connecting with spacer at 5′-O or/and 3′-O of the terminal nucleotide.

In certain embodiments, CpG is: 5′-GGGGGGGGGGGACGATCGTCGGGGGGGGGG-3′ (CMP-001); connecting at 5′-O or/and 3′-O of the terminal nucleotide.

In certain embodiments of the compounds of formulae (I), (V), (V-A), (V-B), (VI), (VII), (VII-A), (VII-B), (VIII), (X-1), (XI), (XI-A), (XI-B), (XII), (XII-A), (XII-B), (XIII), (XIII-A), (XIII-B), (XIV), (XIV-A), (XIV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A) and (XXVII-B), wherein CpG comprises a following formula:

5′N₁X₁CGX₂N₂3′
wherein at least one nucleotide separates consecutive CpGs; X₁ is adenine, guanine, or thymine; X₂ is cytosine or thymine; N is any nucleotide and N₁+N₂ is from about 0-26 bases with the proviso that N₁ and N₂ does not contain a CCGG quadmer or more than one CCG or CGG trimer; the nucleic acid sequence is from about 8-30 bases in length; and
CpG is connecting with spacer at 5′-O or/and 3′-O of the terminal nucleotide.

In certain embodiments, CpG is a phosphorothioate linked 5′-T*C*G*T*C*G*T*T*T*T*G*T*C*G*T*T*T*T*G*T*C*G*T*T-3′ (PF-3512676); connecting at 5′-O or/and 3′-O of the terminal nucleotide.

In certain embodiments of the compounds of formulae (I), (V), (V-A), (V-B), (VI), (VII), (VII-A), (VII-B), (VIII), (X-1), (XI), (XI-A), (XI-B), (XII), (XII-A), (XII-B), (XIII), (XIII-A), (XIII-B), (XIV), (XIV-A), (XIV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A) and (XXVII-B), wherein CpG comprises the following formula:

5′-Nₓ(TCG(N_{q}))_{y}N_{w}(X₁X₂CGX₂′X₁′(CG)ₚ)_{z} a)
wherein N are nucleosides, x=0, y=1, w=0, p=0 or 1, q=0, 1 or 2, and z=1-20, X₁ and X₁′ are self-complimentary nucleosides, X₂ and X₂′ are self-complimentary nucleosides, and wherein the 5′ T of the (TCG(N_{q}))_{y} sequence is positioned at the 5′ end of the polynucleotide; and
b) a palindromic sequence at least 8 bases in length wherein the palindromic sequence comprises the first (X₁X₂CGX₂′X₁′) of the (X₁X₂CGX₂′X₁′(CG)ₚ)_{z} sequences, wherein the polynucleotide is at least 15 bases in length; and
CpG is connecting with spacer at 5′-O or/and 3′-O of the terminal nucleotide.

In certain embodiments, CpG is a phosphorothioate linked 5′-T*C*G *A*A*C *G*T*T *C*G*A *A*C*G *T*T*C *G*A*A *C*G*T *T*C*G *A*A*T-3′ (SD-101); connecting at 5′-O or/and 3′-O of the terminal nucleotide.

In certain embodiments of the compounds of formulae (I), (V), (V-A), (V-B), (VI), (VII), (VII-A), (VII-B), (VIII), (X-1), (XI), (XI-A), (XI-B), (XII), (XII-A), (XII-B), (XIII), (XIII-A), (XIII-B), (XIV), (XIV-A), (XIV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A) and (XXVII-B), wherein CpG comprises at least two oligonucleotides linked together at their 3′ ends, an internucleotide linkage, or a functionalized nucleobase or sugar by a non-nucleotidic linker, wherein at least one of the oligonucleotides is an immunostimulatory oligonucleotide having an accessible 5′ end and comprising an immunostimulatory dinucleotide selected from the group consisting of CG, C^{#}G, CG^{#}, and C^{#}G^{#}, wherein C is cytidine or 2′-deoxycytidine, C^{#} is 2′-deoxythymidine, arabinocytidine, 2′-deoxy-2′-substituted arabinocytidine, 2′-O-substituted arabinocytidine, 2′-deoxy-5-hydroxycytidine, 2′-deoxy-N4-alkyl-cytidine, 2′-deoxy-4-thiouridine or other non-natural pyrimidine nucleoside, G is guanosine or 2′-deoxyguanosine, G^{#} is 2′-deoxy-7-deazaguanosine, 2′-deoxy-6-thioguanosine, arabinoguanosine, 2′-deoxy-2′ substituted-arabinoguanosine, 2′-O-substituted-arabinoguanosine, or other non-natural purine nucleoside, and p is an internucleoside linkage selected from the group consisting of phosphodiester, phosphorothioate, and phosphorodithioate; and
CpG is connecting with spacer at one or two 5′-0 of the terminal nucleotide or/and internucleotide linkage.

In certain embodiments, CpG is a phosphorothioate linked 5′-T*C*G₁*A*A*C*G₁*T*T*C*G₁*-X-*G₁*C*T*T*G₁*C*A*A*G₁*C*T*-5′, wherein X is a glycerol linker and GI is 2′-deoxy-7-deazaguanosine (IMO-2125); connecting at one or two of the terminal nucleotide or/and glycerol.

In certain embodiments of the conjugates, TLR9 agonist derivative or STING agonist derivative of formulae (X), (X-1), (X-A), (X-B), (X-C), (X-D), (X-E), (X-F), (X-G), (X-H), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) and (XXVIV-C), wherein the triazole functional groups have the following structure:

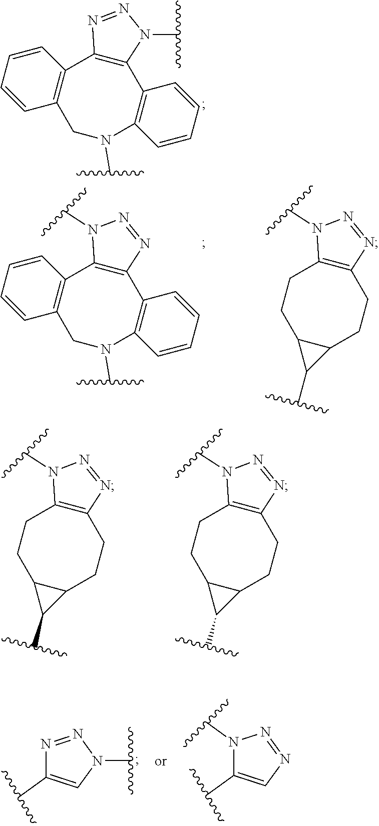

In certain embodiments of the conjugates, TLR9 agonist derivative or STING agonist derivative of formulae (X), (X-1), (X-A), (X-B), (X-C), (X-D), (X-E), (X-F), (X-G), (X-H), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) and (XXVIV-C), wherein the triazole functional groups have the following structure:

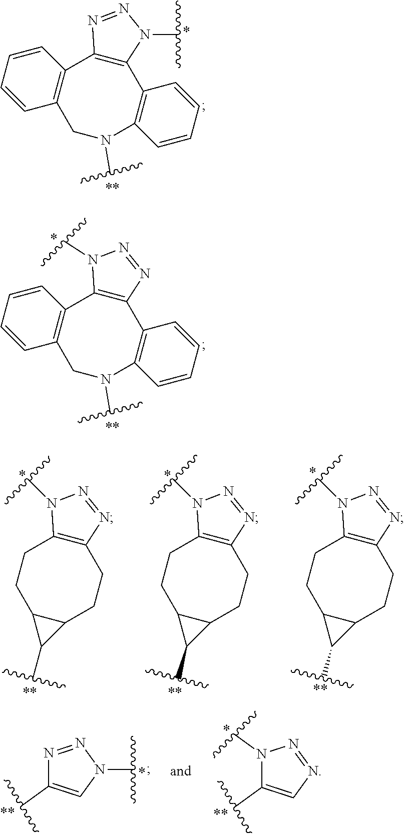

In certain embodiments, triazole functional group is

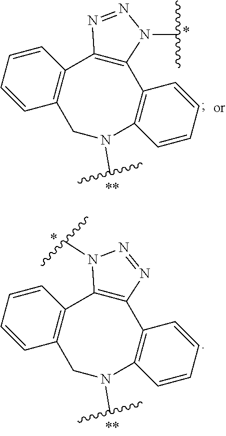 [SMILES: *N1Cc2ccccc2-c2nnn(*)c2-c2ccccc21 ; *N1Cc2ccccc2-c2c(nnn2*)-c2ccccc21]

In certain embodiments, of the conjugate of formula (X) or conjugate of formula (X-1) T is connected to —Z²-A² through * and T is connected to —Z or —Z³-through **. In certain embodiments, T is connected to —Z²-A² through ** and T is connected to —Z¹— or —Z³— through*.

In certain embodiments, the conjugates, TLR9 agonist derivative or STING agonist derivative of formulae (X-A), (X-B), (X-C), (X-D), (X-E), (X-F), (X-G), (X-H), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) and (XXVIV-C), T is connected to X² through ** and X¹ through *. In certain embodiments, T is connected to X² through * and X¹ through **. In certain embodiments, T is connected to X⁴ through ** and X¹ through *. In certain embodiments, T is connected to X⁴ through * and X¹ through **. In certain embodiments, T is connected to X³ through ** and X¹ through *. In certain embodiments, T is connected to X³ through * and X¹ through **.

In certain embodiments, the conjugates, TLR9 agonist derivative or STING agonist derivative of formulae (X-A), (X-B), (X-C), (X-D), (X-E), (X-F), (X-G), (X-H), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) and (XXVIV-C), a variable that can exist more than once (e.g., T, Z², and A² in formula (X)) or a variable that is defined in groups (e.g., Z¹, Z², and Z³ in formula (X)) can be same or different. At each occurrence of a variable, the selection is made independently from other variables present in the same formula, even if the same variable exist in duplicate or more.

In certain embodiments, the present disclosure provides a method for preparing Drug-Drug conjugates according to scheme (II):

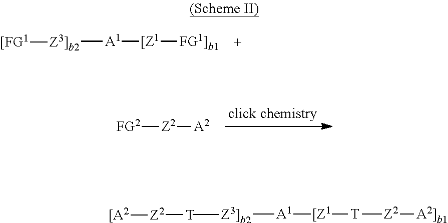

wherein:
b1 is an integer of 0 or 1;
b2 is an integer of 0 or 1;
with the proviso that b1+b2 is 1 or 2;
each T is independently a triazole functional group;
Z¹, Z², and Z³ are each independently a spacer moiety;
A¹ and A² are each independently a therapeutic agent or an active moiety of a therapeutic agent; or a compound that decomposes to a therapeutic agent.
FG¹ is a functional group capable of reacting with FG² through click chemistry selected from the group consisting of azide, alkynyl, and cycloalkynyl groups; and
FG² is a functional group capable of reacting with FG¹ through click chemistry selected from the group consisting of azide, alkynyl, and cycloalkynyl groups. In certain embodiments, Z¹, Z², and Z³ are each independently a releasable linker moiety. In certain embodiments, the releasable linker moiety is derived from the releasable linker of formula (II), (II-A), (II-B) and (II-C). In certain embodiments, Z¹, Z², and Z³ are each independently a non-releasable linker moiety. In certain embodiments, the cycloalkynyl is dibenzocyclooctyne (DBCO), or bicyclo[6.1.0]nonyne (BCN).

In certain embodiments of the method for preparing Drug-Drug conjugates, the therapeutic agent is a STING agonist, a TLR9 agonist, or a TLR7/8 agonist. In certain embodiments, the STING agonist is any STING agonist disclosed herein. Exemplary STING agonist are disclosed in WO 2019/043634, ADU-S100, MK-1454, BMS-986301, GSK3745417, E7766, SB11285,

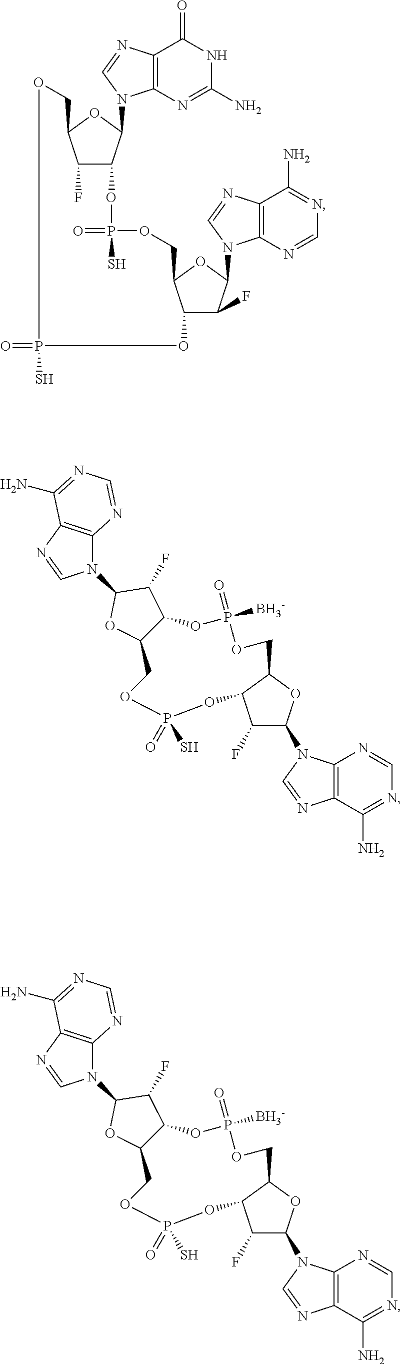

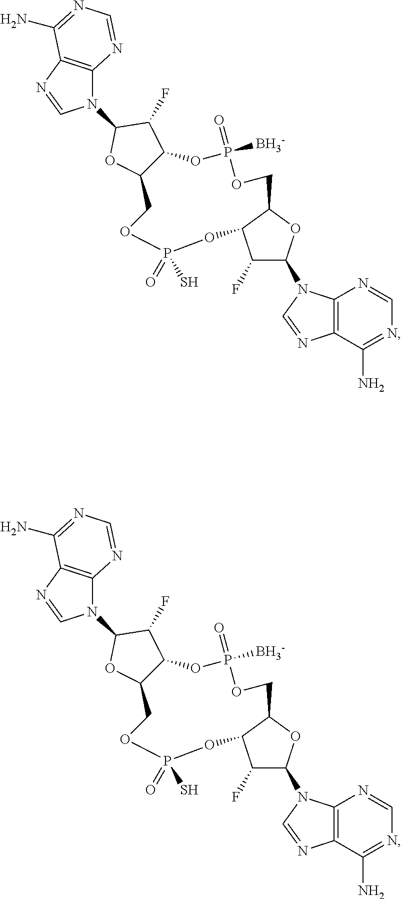

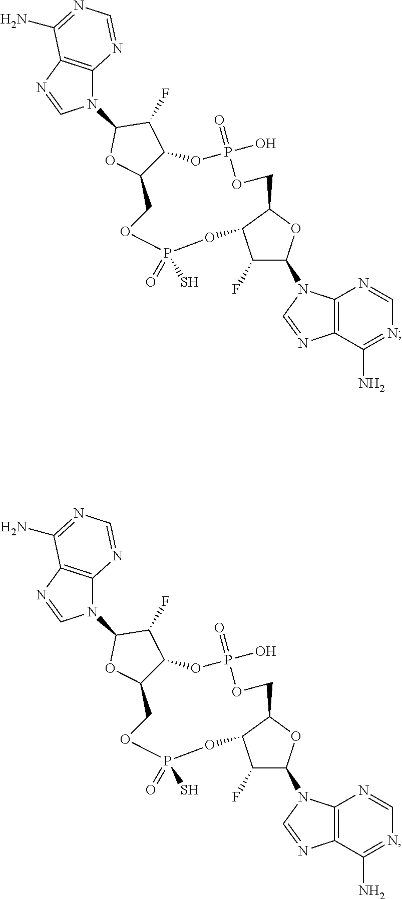 [SMILES: Nc1ncnc2c1ncn2[C@@H]1O[C@@H]2CO[P@@](=O)(S)O[C@H]3[C@@H](F)[C@H](n4cnc5c(N)ncnc54)O[C@@H]3COP(=O)(O)O[C@H]2[C@H]1F ; Nc1ncnc2c1ncn2[C@@H]1O[C@@H]2CO[P@](=O)(S)O[C@H]3[C@@H](F)[C@H](n4cnc5c(N)ncnc54)O[C@@H]3COP(=O)(O)O[C@H]2[C@H]1F]

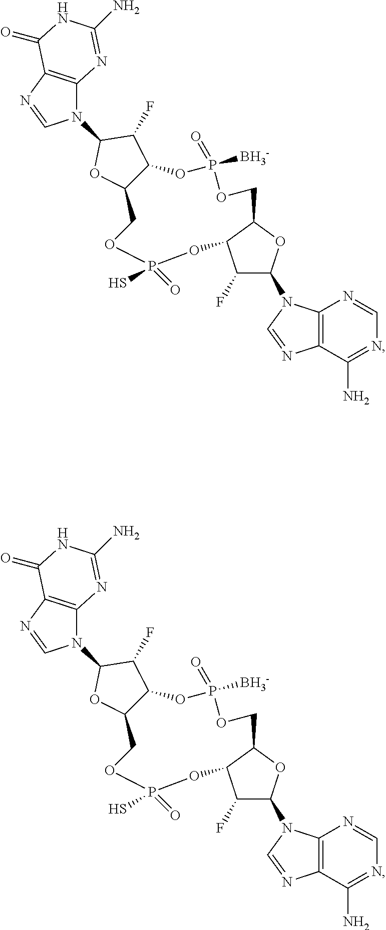

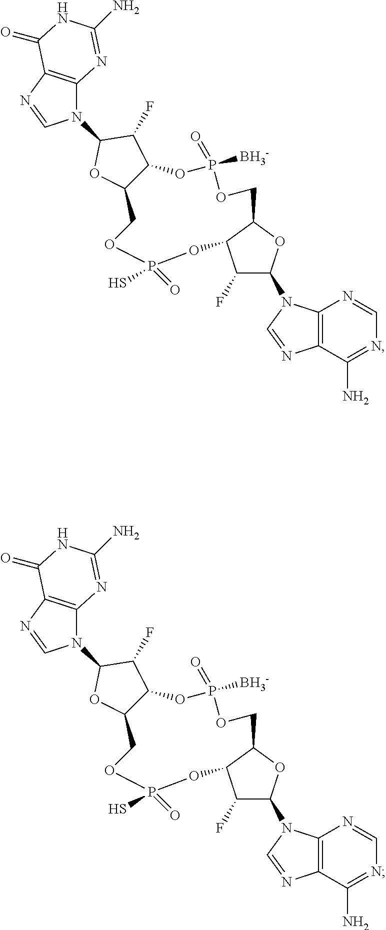

or stereoisomer thereof.

In certain embodiments of the methods for preparing Drug-Drug conjugates, the TLR9 agonist is a TLR9 agonist of formula (I).

In certain embodiments of the methods of preparing Drug-Drug conjugates the TLR7/8 agonist is R848.

In certain embodiments, the present disclosure provides a pharmaceutical composition comprising a compound or conjugate disclosed herein, and one or more pharmaceutically acceptable excipients. In certain embodiments, the pharmaceutical composition is administered for the treatment of cancer, an infection, or an autoimmune disease. In certain embodiments, the pharmaceutical composition is administered in combination with other suitable therapeutic agents.

In certain embodiments, the compounds of the present disclosure comprise a phosphorus atom bonded to a boron atom to form a Lewis acid/Lewis base adduct. The Phosphorus-boron bond may be depicted interchangeably as a coordinate covalent (or dative bond) or as a covalent bond with formal charges.

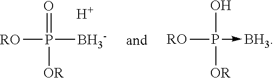

Although one form of the Lewis acid/Lewis base adduct may be depicted herein, all such forms are contemplated within the scope of the disclosure.

When a compound provided herein contains an acidic or basic moiety, it can also be provided as a pharmaceutically acceptable salt. See, Berge et al., J. Pharm. Sci. 1977, 66, 1-19; Handbook of Pharmaceutical Salts: Properties, Selection, and Use, 2nd ed.; Stahl and Wermuth Eds.; John Wiley & Sons, 2011. In certain embodiments, a pharmaceutically acceptable salt of a compound provided herein is a solvate. In certain embodiments, a pharmaceutically acceptable salt of a compound provided herein is a hydrate.

Suitable acids for use in the preparation of pharmaceutically acceptable salts of a compound provided herein include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts of a compound provided herein include, but are not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including, but not limited to, L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, triethanolamine, trimethylamine, triethylamine, N-methyl-D-glucamine, 2-amino-2-(hydroxymethyl)-1,3-propanediol, and tromethamine.

A compound provided herein may also be provided as a prodrug, which is a functional derivative of the compound and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis.

### Pharmaceutical Compositions and Formulations

In one embodiment, the present disclosure relates to a pharmaceutical composition comprising a compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI), (XVI-1), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C) or any subgenera thereof, or a pharmaceutically acceptable salt, stereoisomer, solvate or hydrate thereof, and one or more pharmaceutically acceptable excipients.

The pharmaceutically acceptable excipients and adjuvants are added to the composition or formulation for a variety of purposes. In another embodiment, a pharmaceutical composition comprising one or more compounds of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI), (XVI-1), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C) or any subgenera thereof, or a pharmaceutically acceptable salt, stereoisomer, solvate or hydrate thereof, further comprises a pharmaceutically acceptable carrier. In one embodiment, a pharmaceutically acceptable carrier includes a pharmaceutically acceptable excipient, binder, and/or diluent. In one embodiment, suitable pharmaceutically acceptable excipients include, but are not limited to, water, salt solutions, alcohol, polyethylene glycols, gelatin, lactose, amylase, magnesium stearate, talc, silicic acid, viscous paraffin, hydroxymethylcellulose and polyvinylpyrrolidone.

In one embodiment, excipients can be used to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present disclosure have improved cellular potency, pharmacokinetic properties, as well as improved oral bioavailability.

In certain embodiments, the pharmaceutical compositions of the present disclosure may additionally contain other adjunct components conventionally found in pharmaceutical compositions, at their art-established usage levels. Thus, for example, the pharmaceutical compositions may contain additional, compatible, pharmaceutically-active materials such as, for example, antipruritics, astringents, local anesthetics or anti-inflammatory agents, or may contain additional materials useful in physically formulating various dosage forms of the compositions of the present disclosure, such as dyes, flavoring agents, preservatives, antioxidants, opacifiers, thickening agents and stabilizers. However, such materials, when added, should not unduly interfere with the biological activities of the components of the compositions of the present disclosure. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents.

Liquid formulations may also be prepared by the reconstitution of a solid.

The compounds of the present disclosure may also be used in fast-dissolving, fast-disintegrating dosage forms such as those described in Expert Opinion in Therapeutic Patents, 11 (6), 981986 by Liang and Chen (2001), the disclosure of which is incorporated herein by reference in its entirety.

For tablet dosage forms, depending on dose, the drug may make up from 1 wt % to wt % of the dosage form, more typically from 5 wt % to 60 wt % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant will comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colorants, flavoring agents, preservatives and taste-masking agents.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in “Pharmaceutical Dosage Forms: Tablets, Vol. 1”, by H. Lieberman and L. Lachman, Marcel Dekker, N.Y., N.Y., 1980 (ISBN 0-8247-6918-X).

The foregoing formulations for the various types of administration discussed herein may be in an immediate and/or modified release formulation. Modified release formulations include delayed-, sustained-, pulsed-, controlled-, targeted and programmed release. Suitable modified release formulations for the purposes of the disclosure are described in U.S. Pat. No. 6,106,864. Details of other suitable release technologies such as high energy dispersions and osmotic and coated particles are to be found in Verma et al, Pharmaceutical Technology On-line, 25(2), 1-14 (2001). The use of chewing gum to achieve controlled release is described in WO 00/35298.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Injection solutions and suspensions may be prepared from sterile powders, granules and tablets of the kind previously described.

The solubility of compounds of the present disclosure used in the preparation of parenteral solutions may be increased by the use of appropriate formulation techniques, such as the incorporation of solubility-enhancing agents.

Formulations for parenteral administration may be in an immediate and/or modified release formulation. Thus, compounds of the disclosure may be formulated as a solid, semi-solid, or thixotropic liquid for administration as an implanted depot providing modified release of the active compound. Examples of such formulations include drug-coated stents and poly (glycolideco-dl-lactide) or PGLA microspheres.

The compounds of the disclosure may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubilizer. Most commonly used for these purposes are alpha-, beta- and gammacyclodextrins, examples of which may be found in International Patent Applications WO 91/11172, WO 94/02518 and WO 98/55148.

Because of their potential use in medicine, the salts of the compounds of this disclosure are preferably pharmaceutically acceptable. In one embodiment, the salt of the compounds of the present disclosure in a pharmaceutical formulation or a pharmaceutical composition is a pharmaceutically acceptable salt. Suitable pharmaceutically acceptable salts include, but are not limited to, those described by P. Heinrich Stahl and Camille G. Wermuth in Handbook of Pharmaceutical Salts: Properties, Selection, and Use, 2nd ed. (Wiley-VCH: 2011) and also Remington's Pharmaceutical Sciences, 18th ed. (Mack Publishing, Easton PA: 1990) and also Remington: The Science and Practice of Pharmacy, 19th ed. (Mack Publishing, Easton PA: 1995). Salt encompassed within the term “pharmaceutically acceptable salts” refer to non-toxic salts of the compounds in this disclosure.

Salts of the compounds of this disclosure containing a basic amine or other basic functional group may be prepared by any suitable method known in the art, including treatment of the free bases with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, formic acid, alginic acid, pyruvic acid, oxalic acid, glycolic acid, salicylic acid, pyranosildyl acid, such as glucuronic acid or galacturonic acid, alphahydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, phenylacetates, phenylpropionates, phenylbutrates, citrates, lactates, glycolate, resinate, lactates, camsylates, tartrates, mandelates, and sulfonates, such as xylenesulfonates, methanesulfonates, propanesulfonates, naphthalene-1-sulfonates and naphthalene-2-sulfonates.

Salts of the compounds of this disclosure can be prepared by reacting with a suitable base. Pharmaceutically acceptable salts include, but are not limited to: alkali metal salts (especially sodium and potassium), alkaline earth metal salts (especially calcium and magnesium), aluminum salts and ammonium salts, zinc, as well as salts made from physiologically acceptable organic bases such as diethylamine, isopropylamine, olamine, benzathine, benethamine, tromethamine (2-amino-2-(hydroxymethyl)propane-1,3-diol), morpholine, epolamine, piperidine, piperazine, picoline, dicyclohexylamine, N,N′-dibenzylethylenediamine, 2-hydroxyethylamine, tri-(2-hydroxyethyl)amine, chloroprocaine, choline, deanol, imidazole, diethanolamine, ethylenediamine, meglumine (N-methylglucamine), procaine, dibenzylpiperidine, dehydroabietylamine, glucamine, collidine, quinine, quinolone, erbumine and basic amino acids such as lysine and arginine. Additional pharmaceutically acceptable salts are known to those skilled in the art.

### Therapeutic Use

In certain embodiments, the present disclosure relates to therapeutic methods in the treatment of diseases and conditions in which modulation of STING (Stimulator of Interferon Genes) and/or TLR is beneficial. In one embodiment, the present disclosure relates to a method of treating a disease or a condition in which the modulation of STING and/or TLR is beneficial in a subject, comprising administering a therapeutically effective amount of a compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C), or any subgenera thereof, or a pharmaceutically acceptable salt, stereoisomer, solvate or hydrate thereof, to the subject in need thereof. In one embodiment, the present disclosure relates to a compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C), or any subgenera thereof, or a pharmaceutically acceptable salt, stereoisomer, solvate or hydrate thereof, for use in treating a disease or a condition in which the modulation of STING and/or TLR is beneficial in a subject.

In one embodiment, the present disclosure relates to a method modulating STING and/or TLR, comprising administering a therapeutically effective amount of a compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C) or any subgenera thereof, or a pharmaceutically acceptable salt, stereoisomer, solvate or hydrate thereof, to the subject in need thereof. In one embodiment, the present disclosure relates to compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C) or any subgenera thereof, or a pharmaceutically acceptable salt, stereoisomer, solvate or hydrate thereof, for use in modulating STING and/or TLR.

In certain embodiments, the present disclosure relates to a method for inducing, modifying or stimulating an appropriate immune response in a mammal comprising administering to the mammal a therapeutically effective amount of a compound of the disclosure, or pharmaceutically acceptable salt thereof. In one embodiment, the present disclosure relates to compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C), or any subgenera thereof, or a pharmaceutically acceptable salt, stereoisomer, solvate or hydrate thereof, for use in inducing, modifying or stimulating an appropriate immune response in a mammal. The immune response can comprise, without limitation, specific immune response, non-specific immune response, both specific and non-specific response, innate response, primary immune response, adaptive immunity, secondary immune response, memory immune response, immune cell activation, immune cell proliferation, immune cell differentiation, and cytokine expression.

In certain embodiments, the compounds of the present disclosure induce STING- and/or TLR dependent type I interferon production in a subject (e.g., a human).

In certain embodiments, the diseases or conditions in which modulation of STING and/or TLR is beneficial is cancer.

In certain embodiments, the compounds of the present disclosure can be useful in the treatment of cancer. In one embodiment, the present disclosure relates to a method of treating cancer, comprising administering a therapeutically effective amount of a compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C), or any subgenera thereof, or a pharmaceutically acceptable salt, stereoisomer, solvate or hydrate thereof, to the subject in need thereof. In one embodiment, the present disclosure relates to a compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C), or any subgenera thereof, or a pharmaceutically acceptable salt, stereoisomer, solvate or hydrate thereof, for use in treating cancer. Non-limiting examples of cancer include, colorectal cancer, an aero-digestive squamous cancer, a lung cancer, a brain cancer, a liver cancer, a stomach cancer, a bladder cancer, a thyroid cancer, an adrenal cancer, a gastrointestinal cancer, an oropharyngeal cancer, an esophageal cancer, a head and neck cancer, an ovarian cancer, a uterine cancer, a cervical cancer, an endometrial cancer, a breast cancer, a melanoma, a prostate cancer, a pancreatic carcinoma, a renal carcinoma, a sarcoma, a leukemia, a lymphoma and a multiple myeloma.

In one aspect, the present disclosure provides a plurality of methods of treating cancer in a mammalian subject in need thereof, comprising administering a therapeutically effective amount of a pharmaceutical composition comprising a conjugate or compound comprising a STING and/or TLR agonist, as disclosed herein. In certain embodiments, the present disclosure provides methods of treating cancer in a mammalian subject in need thereof, comprising administering to the subject a therapeutically effective amount of a pharmaceutical composition comprising a conjugate or compound disclosed herein by intratumoral delivery. In another aspect of the method, intratumoral delivery comprises injection of the pharmaceutical composition into at least one tumor lesion. In other aspects, treating cancer comprises inducing accumulation of tumor antigen-specific T cells in the injected tumor, for example, at greater numbers than had the pharmaceutical composition been administered at an extratumoral site. In other aspects, treating cancer comprises eliciting a systemic, tumor antigen-specific T cell response, including for example, a systemic, tumor antigen-specific T cell response of a higher magnitude than had the immunogenic composition been administered at an extratumoral site. In other aspects, treating cancer comprises eliciting a systemic tumor antigen-specific T cell response. In other aspects, treating cancer comprises reducing numbers of CD4+ FoxPS+ regulatory T cells in the injected tumor. In other aspects, the subject has one or more uninjected tumors (primary or metastatic lesions) in addition to the injected tumor, and treating cancer comprises one or more of the following: (a) reducing the number of uninjected tumors; (b) reducing the volume of uninjected tumors; and (c) retarding the growth of uninjected tumors. In some aspects, treating cancer comprises one or more of the following: (d) increasing the survival time of the subject; (e) reducing the volume of the injected tumor; and (f) retarding the growth of the injected tumor. In certain embodiments, when the cancer is a solid tumor, “treating” cancer comprises shrinking the size of the solid tumor and any metastatic lesions, or otherwise reducing viable cancer cell numbers. In other embodiments, when the cancer is a solid tumor, “treating” cancer comprises delaying growth of the solid tumor and any metastatic lesions. In some aspects, treating cancer comprises increasing progression free survival or increasing time to progression. In other embodiments, the method further comprises administering an effective amount of a second, or additional, therapeutic agents to the subject. In certain embodiments, “treating” cancer means to bring about a beneficial clinical result, such as causing remission or otherwise prolonging survival as compared to expected survival in the absence of treatment. In certain embodiments, “treating cancer” comprises assessing a patient's response to the immunogenic composition according to the Response Evaluation Criteria in Solid Tumors (RECIST version 1.1) as described (see, e.g., Eisenhauer et al 2009 Eur J Cancer 45:228-247). Response criteria to determine objective anti-tumor responses per RECIST include: complete response, partial response, progressive disease, and stable disease. In certain embodiments, the tumor is a sarcoma, a carcinoma, or an actinic keratosis. In certain embodiments, the tumor is a lymphoma. In certain embodiments, the cancer is selected from the group consisting of breast cancer, prostate cancer, lung cancer, colorectal cancer, uterine cancer, bladder cancer, melanoma, head and neck cancer, non-Hodgkin lymphoma, kidney cancer, ovarian cancer, pancreatic cancer, and thyroid cancer. In certain embodiments, the cancer is a primary cancer of a site selected from the group consisting of oral cavity, digestive system, respiratory’ system, skin, breast, genital system, urinary system, ocular system, nervous system, endocrine system, and lymphoma.

In certain embodiments, the method further comprises administering an effective amount of a second therapeutic agent to the subject. In some of these embodiments, the second therapeutic agent comprises a chemotherapeutic agent selected from the group consisting of actinomycin, afatinib, alectimb, asparaginase, azacitidine, azathioprine, bicalutamide, binimetinib, bleomycin, bortezomib, camptothecin, carbopktin, capecitabine, carmustine, certinib, cisplatin, chlorambucil, cobimetinib, crizotinib, cyclophosphamide, cytarabine, dabrafenib, dacarbazine, daunorubiein, docetaxel, doxifluridine, doxorubicin, encorafemb, erlotimb, epirubicin, epotlnlone, etoposide, fludarabine, flutamine, fluorouracil, gefkinib, gemcitabme, hydroxyurea, idarubicin, ifosfamide, imatinib, irinotecan, lapatinib, letrozole, mechlorethamine, mercaptopurine, methotrexate, mitomycin, mitoxantrone, octreotide, oxaliplatin, paclitaxel, pemetrexed, rakitrexed, sorafenib, sunitinib, tamoxifen, temozoionnde, teniposide, tioguamne, topotecan, trametimb, valrubicin, vemurafenib, vinblastine, vincristine, vindesine, vinorelbine, and combinations thereof. In certain embodiments, the second therapeutic agent comprises one or both of a BRAF inhibitor and a MEK inhibitor. In certain embodiments, the second therapeutic agent comprises a epigenetic modulator selected from the group consisting of HDAC inhibitors (see e g., voronistat [SAHA], ronudepsin, entinostat, abexinostat, elinostat [CHR-3996], panobinostat, quisrnostat [JNJ-26481585], 4SC-202, resminostat [SB939], pracmostat [CI-9940], and valproate), DNAmethyltransferase inhibitors (see e g., azacytidme, decitabine, zebularine, SGI-1027, RG-108, and smfungin), and combinations thereof. In some of these embodiments, the second therapeutic agent is an antagonist of an inhibitory immune checkpoint molecule, for example, an inhibitory immune checkpoint molecule selected from the group consisting of PD-I, PD-L1, PD-L2, CTLA-4 (CD152), LAG-3, TIM-3, TIGIT, IL-10, indoleamine 2,3-dioxygenase (IDO), P-selectin glycoprotein ligand-1 (PSGL-1), and TGF-beta. In some of these embodiments, the second therapeutic agent is an agonist of an immune stimulatory molecule. In some of these embodiments, the immune stimulatory molecule is selected from the group consisting of CD27, CD4Q, 0X40 (CD 134), GITR, 4-IBB (CD 137), CD28, and ICOS (CD278), In some of these embodiments, the second therapeutic agent comprises an antibody, fragment, or derivative thereof. In some of these embodiments, the second therapeutic agent is an antagonist of an inhibitory immune checkpoint molecule and the second therapeutic agent comprises an antibody, fragment, or derivative thereof. In certain embodiments, the method further comprises administering radiation therapy and/or administering an effective amount of a second therapeutic agent to the subject. In some of these embodiments, the effective amount of the immunogenic composition and the effective amount of the second therapeutic agent together result in an additive effect or better against the tumor. In some of these embodiments, the effective amount of the immunogenic composition and the effective amount of the second therapeutic agent together result in a synergistic effect against the tumor.

In one embodiment, the present disclosure relates to a method of treating a disease, comprising administering a therapeutically effective amount of a compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C), or any subgenera thereof, or a pharmaceutically acceptable salt, stereoisomer, solvate or hydrate thereof, to the subject in need thereof, wherein the disease is selected from cancer, rheumatoid arthritis, psoriasis, acute rejection of an organ transplant, allergic asthma or Crohn's disease. In one embodiment, the present disclosure relates to a compound of formulae (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C), or any subgenera thereof, or a pharmaceutically acceptable salt, stereoisomer, solvate or hydrate thereof, for use in treating a disease, wherein the disease is selected from cancer, rheumatoid arthritis, psoriasis, acute rejection of an organ transplant, allergic asthma or Crohn's disease.

In one embodiment, the diseases or conditions in which modulation of STING and/or TLR is beneficial are neurological disorders. In certain embodiments, the compounds of the present disclosure can be useful in the treatment of a neurological disorder, which includes, but is not limited to, disorders that involve the central nervous system (brain, brainstem and cerebellum), the peripheral nervous system (including cranial nerves), and the autonomic nervous system (parts of which are located in both central and peripheral nervous system). Non-limiting examples of cancer include acquired epileptiform aphasia; acute disseminated encephalomyelitis; adrenoleukodystrophy; age-related macular degeneration; agenesis of the corpus callosum; agnosia; Aicardi syndrome; Alexander disease; Alpers' disease; alternating hemiplegia; Alzheimer's disease; Vascular dementia; amyotrophic lateral sclerosis; anencephaly; Angelman syndrome; angiomatosis; anoxia; aphasia; apraxia; arachnoid cysts; arachnoiditis; Anronl-Chiari malformation; arteriovenous malformation; Asperger syndrome; ataxia telegiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome; causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy; chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease; cytomegalovirus infection; dancing eyes-dancing feet syndrome; Dandy-Walker syndrome; Dawson disease; De Morsier's syndrome; Dejerine-Klumke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; fronto-temporal dementia and other “tauopathies”; Gaucher's disease; Gerstmann's syndrome; giant cell arteritis; giant cell inclusion disease; globoid cell leukodystrophy; Guillain-Barre syndrome; HTLV-1-associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile phytanic acid storage disease; infantile refsum disease; infantile spasms; inflammatory myopathy; intracranial cyst; intracranial hypertension; Joubert syndrome; Kearns-Sayre syndrome; Kennedy disease Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gustaut syndrome; Lesch-Nyhan syndrome; leukodystrophy; Lewy body dementia; Lissencephaly; locked-in syndrome; Lou Gehrig's disease (i.e., motor neuron disease or amyotrophic lateral sclerosis); lumbar disc disease; Lyme disease—neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neuron disease; Moyamoya disease; mucopolysaccharidoses; multi-infarct dementia; multifocal motor neuropathy; multiple sclerosis and other demyelinating disorders; multiple system atrophy with postural hypotension; p muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenital; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; post-polio syndrome; postherpetic neuralgia; postinfectious encephalomyelitis; postural hypotension; Prader-Willi syndrome; primary lateral sclerosis; prion diseases; progressive hemifacial atrophy; progressive multifocal leukoencephalopathy; progressive sclerosing poliodystrophy; progressive supranuclear palsy; pseudotumor cerebri; Ramsay-Hunt syndrome (types I and II); Rasmussen's encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus dance; Sandhoff disease; Schilder's disease; schizencephaly; septo-optic dysplasia; shaken baby syndrome; shingles; Shy-Drager syndrome; Sjögren's syndrome; sleep apnea; Soto's syndrome; spasticity; spina bifida; spinal cord injury; spinal cord tumors; spinal muscular atrophy; Stiff-Person syndrome; stroke; Sturge-Weber syndrome; subacute sclerosing panencephalitis; subcortical arteriosclerotic encephalopathy; Sydenham chorea; syncope; syringomyelia; tardive dyskinesia; Tay-Sachs disease; temporal arteritis; tethered spinal cord syndrome; Thomsen disease; thoracic outlet syndrome; Tic Douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau disease; Wallenberg's syndrome; Werdnig-Hoffman disease; West syndrome; whiplash; Williams syndrome; Wildon's disease; and Zellweger syndrome.

In one embodiment, the diseases or conditions in which modulation of STING and/or TLR is beneficial are autoimmune diseases and disorders. In certain embodiments, the compounds of the present disclosure can be useful in the treatment of an autoimmune disease. Non-limiting examples include rheumatoid arthritis, systemic lupus erythematosus, multiple sclerosis, inflammatory bowel diseases (IBDs) comprising Crohn disease (CD) and ulcerative colitis (UC), which are chronic inflammatory conditions with polygenic susceptibility. In certain embodiments, the condition is an inflammatory bowel disease. In certain embodiments, the condition is Crohn's disease, autoimmune colitis, iatrogenic autoimmune colitis, ulcerative colitis, colitis induced by one or more chemotherapeutic agents, colitis induced by treatment with adoptive cell therapy, colitis associated by one or more alloimmune diseases (such as graft-vs-host disease, e.g., acute graft vs. host disease and chronic graft vs. host disease), radiation enteritis, collagenous colitis, lymphocytic colitis, microscopic colitis, and radiation enteritis. In certain of these embodiments, the condition is alloimmune disease (such as graft-vs-host disease, e.g., acute graft vs. host disease and chronic graft vs. host disease), celiac disease, irritable bowel syndrome, rheumatoid arthritis, lupus, scleroderma, psoriasis, cutaneous T-cell lymphoma, uveitis, and mucositis (e.g., oral mucositis, esophageal mucositis or intestinal mucositis).

In one embodiment, the present disclosure relates to modulation of the immune system by STING and/or TLR comprising administering a therapeutically effective amount of the compounds of the present disclosure. In certain embodiments, modulation of the immune system by STING and/or TLR provides for the treatment of diseases, such as diseases caused by foreign agents. Exemplary infections by foreign agents which may be treated and/or prevented by the method of the present disclosure include an infection by a bacterium (e.g., a Gram-positive or Gram-negative bacterium), an infection by a fungus, an infection by a parasite, and an infection by a virus. In one embodiment of the present disclosure, the infection is a bacterial infection (e.g., infection by E. coli, Klebsiella pneumoniae, Pseudomonas aeruginosa, Salmonella spp., Staphylococcus aureus, Streptococcus spp., or vancomycin-resistant Enterococcus). In another embodiment, the infection is a fungal infection (e.g. infection by a mold, an yeast, or a higher fungus). In still another embodiment, the infection is a parasitic infection (e.g., infection by a single-celled or multicellular parasite, including Giardia duodenalis, Cryptosporidium parvum, Cyclospora cayetanensis, and Toxoplasma gondiz). In yet another embodiment, the infection is a viral infection (e.g., infection by a virus associated with AIDS, avian flu, chickenpox, cold sores, common cold, gastroenteritis, glandular fever, influenza, measles, mumps, pharyngitis, pneumonia, rubella, SARS, and lower or upper respiratory tract infection (e.g., respiratory syncytial virus).

In one embodiment, the disease or condition in which modulation of STING and/or TLR is beneficial is hepatitis B. In certain embodiments, the compounds of the present disclosure can be useful in the treatment of hepatitis B (see, e.g., WO 2015/061294).

In one embodiment, the disease or condition in which modulation of STING and/or TLR is beneficial is mucositis. In certain embodiments, the compounds of the present disclosure can be useful in the treatment of mucositis, also known as stomatitis, which can occur as a result of chemotherapy or radiation therapy, either alone or in combination as well as damage caused by exposure to radiation outside of the context of radiation therapy.

In one embodiment, the disease or condition in which modulation of STING and/or TLR is beneficial is uveitis. In certain embodiments, the compounds of the present disclosure can be useful in the treatment of uveitis, which is inflammation of the uvea (e.g., anterior uveitis, e.g., iridocyclitis or iritis; intermediate uveitis (also known as pars planitis); posterior uveitis; or chorioretinitis, e.g., pan-uveitis).

Combination Therapy

In certain embodiments, the methods described herein can further comprise administering one or more additional therapies (e.g., one or more additional therapeutic agents and/or one or more therapeutic regimens) in combination with administration of the compounds of the present disclosure.

The compounds or pharmaceutical compositions of the present disclosure may be co-administered with one or more therapeutically active agent. The term “co-administration” or “coadministration” refers to administration of (a) compound of formula (I), (X), (X-1), (X-A), (X-B), (X-C), (X-D), (X-E), (X-F), (X-G), (XI), (XI-A), (XI-B), (XII), (XII-A), (XII-B), (XIII), (XIII-A), (XIII-B), (XIV), (XIV-A), (XIV-B), (XV), (XV-A), (XV-B), (XVI-1), (XVI), (XVI-A), (XVI-B), (XVII), (XVII-A), (XVII-B), (XVIII), (XVIII-A), (XVIII-B), (XIX), (XIX-A), (XIX-B), (XX), (XXI), (XXIV), (XXV), (XXVI), (XXVI-A), (XXVI-B), (XXVII), (XXVII-A), (XXVII-B), (XXVIII), (XXVIV), (XXVIV-A), (XXVIV-B) or (XXVIV-C), or any subgenera thereof, or a pharmaceutically acceptable salt, stereoisomer, solvate, or hydrate thereof, and (b) at least one additional therapeutically active agent, together in a coordinated fashion. For example, the co-administration can be simultaneous administration, sequential administration, overlapping administration, interval administration, continuous administration, or a combination thereof. In one embodiment, the compound of the present disclosure and at least one additional therapeutically active agent are formulated into a single dosage form. In another embodiment, the compound of the present disclosure and at least one additional therapeutically active agent are provided in a separate dosage forms.

In certain embodiments, the one or more additional therapeutic agent is selected from the group consisting of an immune checkpoint inhibitor (e.g. CTLA-4, PD-1, Tim-3, Vista, BTLA, LAG-3 and TIGIT pathway antagonists; PD-1 pathway blocking agents; PD-L1 inhibitors; including without limitation anti-PD-1 antibodies nivolumab, pembrolizumab or pidilizumab; PD-1 inhibitor AMP-224; anti-CTLA-4 antibody ipilimumab; and anti-PD-L 1 antibodies BMS-936559, MPDL3280A, MEDI4736, or avelumab); a TLR agonist (e.g. CpG or monophosphoryl lipid A); an inactivated or attenuated bacteria which induce innate immunity (e.g., inactivated or attenuated Listeria monocytogenes); a composition that mediates innate immune activation via Toll-like Receptors (TLRs), via (NOD)-like receptors (NLRs), via Retinoic acid inducible gene-based (RIG)-I-like receptors (RLRs), via C-type lectin receptors (CLRs), or via pathogen-associated molecular patterns (PAMPs); and a chemotherapeutic agent.

In certain embodiments, the compound of the present disclosure can be used in combination with a Toll like receptor agonist. The term “Toll like receptor” (or “TLR”) as used herein refers to a member of the Toll-like receptor family of proteins or a fragment thereof that senses a microbial product and/or initiates an adaptive immune response. In one embodiment, a TLR activates a dendritic cell (DC). Toll like receptors (TLRs) are a family of pattern recognition receptors that were initially identified as sensors of the innate immune system that recognize microbial pathogens. TLRs comprise a family of conserved membrane spanning molecules containing an ectodomain of leucine-rich repeats, a transmembrane domain and an intracellular TIR (Toll/IL-1R) domain. TLRs recognize distinct structures in microbes, often referred to as “PAMPs” (pathogen associated molecular patterns). Ligand binding to TLRs invokes a cascade of intra-cellular signaling pathways that induce the production of factors involved in inflammation and immunity. TLR agonists known in the art and finding use in the present disclosure include, but are not limited to, the following: Pam3Cys, a TLR-1/2 agonist; CFA, a TLR-2 agonist; MALP2, a TLR-2 agonist; Pam2Cys, a TLR-2 agonist; FSL-1, a TLR-2 agonist; Hib-OMPC, a TLR-2 agonist; polyribosinic:polyribocytidic acid (Poly I:C), a TLR-3 agonist; polyadenosine-polyuridylic acid (poly AU), a TLR-3 agonist; Polyinosinic-Polycytidylic acid stabilized with poly-L-lysine and carboxymethylcellulose (Hiltonol®), a TLR-3 agonist; monophosphoryl lipid A (MPL), a TLR-4 agonist; LPS, a TLR-4 agonist; bacterial flagellin, a TLR-5 agonist; sialyl-Tn (STn), a carbohydrate associated with the MUC1 mucin on a number of human cancer cells and a TLR-4 agonist; imiquimod, a TLR-7 agonist; resiquimod, a TLR-7/8 agonist; loxoribine, a TLR-7/8 agonist; and unmethylated CpG dinucleotide (CpG-ODN), a TLR-9 agonist.

In certain embodiments, the compounds of the present disclosure as described herein can be used in combination with a chemokine or cytokine. In certain embodiments the chemokine is selected from MCP-1, MCP-2, MCP-3, MCP-24, MCP-5, CXCL76, I-309 (CCL1), BCA1 (CXCL13), MIG, SDF-1/PBSF, IP-10, I-TAC, MIP-1α, MIP-1β, RANTES, eotaxin-1, eotaxin-2, GCP-2, Gro-α, Gro-β, Gro-γ, LARC (CCL20), ELC (CCL19), SLC (CCL21), ENA-78, PBP, TECK(CCL25), CTACK (CCL27), MEC, XCL1, XCL2, HCC-1, HCC-2, HCC-3, or HCC-4. In certain embodiments the cytokine is selected from GM-CSF, IL-1α, IL-1β, IL-2, IL-3, IL-4, IL-5, IL-6, IL-7, IL-8, IL-10, IL-12, IFN-α, IFN-β, IFN-γ, MIP-1α, MIP-1β, TGF-β, TNF-α, or TNF-β. In certain embodiments the cytokine is IL-2.

In certain embodiments, the compound of the present disclosure can be used in combination with therapeutic antibodies. In certain embodiments, the mechanism of action of the therapeutic antibody is Antibody-Dependent Cell-Mediated Cytotoxicity (ADCC). ADCC is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection. Classical ADCC is mediated by natural killer (NK) cells; macrophages, neutrophils and eosinophils can also mediate ADCC. ADCC is an important mechanism of action of therapeutic monoclonal antibodies, including trastuzumab and rituximab, against tumors. Compounds of the present disclosure may act to potentiate ADCC. The following are an exemplary list of antibodies which may be used together with the compounds of the present disclosure. Muromonab-CD3 is used to prevent acute rejection of organ, e.g., kidney transplants. The humanized versions show promise in inhibiting the autoimmune destruction of beta cells in Type 1 diabetes mellitus; Infliximab (Remicade®) and adalimumab (Humira®), which bind to tumor necrosis factor-alpha (TNF-α) and is used in some inflammatory diseases such as rheumatoid arthritis, psoriasis, Crohn's disease; Omalizumab (Xolair®), which binds to IgE thus preventing IgE from binding to mast cells and is used against allergic asthma; Daclizumab (Zenapax®), which binds to part of the IL-2 receptor exposed at the surface of activated T cells and is used to prevent acute rejection of transplanted kidneys; Rituximab (trade name=Rituxan®), which binds to the CD20 molecule found on most B-cells and is used to treat B-cell lymphomas; Ibritumomab (trade name=Zevalin®) is a monoclonal antibody against the CD20 molecule on B cells (and lymphomas) conjugated to isotopes and is given to the lymphoma patient supplemented with Rituxan; Tositumomab (Bexxar®), which is a conjugate of a monoclonal antibody against CD20 and the radioactive isotope iodine-131 (131I); Cetuximab (Erbitux®), which blocks HER1, a receptor for epidermal growth factor (EGF) that is found on some tumor cells (some breast cancers, lymphomas); Trastuzumab (Herceptin®), which blocks HER2, a growth factor receptor over-expressed in some 20% of breast cancers; Adcetris®, which is a conjugate of a monoclonal antibody that binds CD30, a cell-surface molecule expressed by the cells of some lymphomas but not found on the normal stem cells needed to repopulate the bone marrow; Alemtuzumab (Campath-1H®), which binds to CD52, a molecule found on lymphocytes and depletes both T cells and B cells, has produced complete remission of chronic lymphocytic leukemia and shows promise in preventing rejection of kidney transplants; Lym-1 (Oncolym®), which binds to the HLA-DR-encoded histocompatibility antigen that can be expressed at high levels on lymphoma cells; Ipilimumab (Yervoy®), which acts to enhance the body's own immune response to tumors; Vitaxin Binds to a vascular integrin (alpha-v/beta-3) found on the blood vessels of tumors but not on the blood vessels supplying normal tissues; Bevacizumab (Avastin®), which binds to vascular endothelial growth factor (VEGF) preventing it from binding to its receptor and is used for the treatment of colorectal cancers; Abciximab (ReoPro®), which inhibits the clumping of platelets by binding the receptors on their surface that normally are linked by fibrinogen.

Additional therapeutic antibodies that may be used in combination with the compounds of the present disclosure as described herein include a prolactin receptor (PRLR) inhibitor, e.g. as disclosed in U.S. Pat. No. 7,867,493, a HERS inhibitor, e.g. as disclosed in PCT Publication No. WO2012/022814, an EGFR2 and/or EGFR4 inhibitor, e.g. as disclosed in PCT Publication No. WO2014/160160, an M-CSF inhibitor, e.g. as disclosed in PCT Publication No. WO2004/045532, an anti-APRIL antibody, e.g. as disclosed in U.S. Pat. No. 8,895,705, or an anti-SIRPα or anti-CD47 antibody, e.g. as disclosed in U.S. Pat. Nos. 8,728,476 and 8,562,997.

In certain embodiments, the compounds of the present disclosure as described herein can be used in combination with an immune checkpoint inhibitor, such as an immune checkpoint inhibitor selected from the group consisting of a CTLA-4 pathway antagonist, a PD-1 pathway antagonist, a Tim-3 pathway antagonist, a Vista pathway antagonist, a BTLA pathway antagonist, a LAG-3 pathway antagonist, and a TIGIT pathway antagonist.

In certain embodiments, the compounds of the present disclosure are used in combination with chemotherapeutic agents (e.g. small molecule pharmaceutical compounds). Thus, the methods of the present disclosure further involve administering to the subject an effective amount of one or more chemotherapeutic agents as an additional treatment or a combination treatment. In certain embodiments the one or more chemotherapeutic agents is selected from the group consisting of sotrastaurin, nilotinib, 5-(2,4-dihydroxy-5-isopropylphenyl)-N-ethyl-4-(4-(morpholinomethyl)phenyl)isoxazole-3-carboxamide, dactolisib, 8-(6-Methoxy-pyridin-3-yl)-3-methyl-1-(4-piperazin-1-yl-3-trifluoromethyl-phenyl)-1,3-dihydro-imidazo[4,5-c]quinolin-2-one, 3-(2,6-dichloro-3,5-dimethoxyphenyl)-1-(6-((4-(4-ethylpiperazin-1-yl)phenyl)amino)pyrimidin-4-yl)-1-methylurea, buparlisib, 8-(2,6-difluoro-3,5-dimethoxyphenyl)-N-(4-((dimethylamino)methyl)-1H-imidazol-2-yl)quinoxaline-5-carboxamide, (S)-N1-(4-methyl-5-(2-(1,1,1-trifluoro-2-methylpropan-2-yl)pyridin-4-yl)thiazol-2-yl)pyrrolidine-1,2-dicarboxamide, (S)-1-(4-chlorophenyl)-7-isopropoxy-6-methoxy-2-(4-(methyl (((1r,4S)-4-(4-methyl-3-oxopiperazin-1-yl)cyclohexyl)methyl)amino)phenyl)-1,2-dihydroisoquinolin-3 (4H)-one , deferasirox, letrozole, (4 S,5R)-3-(2′-amino-2-morpholino-4′-(trifluoromethyl)-[4,5′-bipyrimidin]-6-yl)-4-(hydroxymethyl)-5-methyloxazolidin-2-one, (S)-5-(5-chloro-1-methyl-2-oxo-1,2-dihydropyridin-3-yl)-6-(4-chlorophenyl)-2-(2,4-dimethoxypyrimidin-5-yl)-1-isopropyl-5,6-dihydropyrrolo[3,4-d]imidazol-4(1H)-one, 4-((2-(((1R,2R)-2-hydroxycyclohexyl)amino)benzo[d]thiazol-6-yl)oxy)-N-methylpicolinamide, imatinib mesylate, 2-fluoro-N-methyl-4-(7-(quinolin-6-ylmethyl)imidazo[1,2-b][1,2,4]triazin-2-yl)benzamide, ruxolitinib, panobinostat, osilodrostat, (S)—N—((S)-1-cyclohexyl-2-4S)-2-(4-(4-fluorobenzoyl)thiazol-2-yl)pyrrolidin-1-yl)-2-oxoethyl)-2-(methylamino)propanamide, (S)-N-((S)-1-cyclohexyl-2-((S)-2-(4-(4-fluorobenzoyl)thiazol-2-yl)pyrrolidin-1-yl)-2-oxoethyl)-2-(methylamino)propanamide, sonidegib phosphate, ceritinib, 7-cyclopentyl-N,N-dimethyl-2-((5-(piperazin-1-yl)pyridin-2-yl)amino)-7H-pyrrolo[2,3-d]pyrimidine-6-carboxamide, N-(4-((1R,3S,5S)-3-amino-5-methylcyclohexyl)pyridin-3-yl)-6-(2,6-difluorophenyl)-5-fluoropicolinamide, 2-(2′,3-dimethyl-[2,4′-bipyridin]-5-yl)-N-(5-(pyrazin-2-yl)pyridin-2-yl)acetamide, encorafenib, 7-cyclopentyl-N,N-dimethyl-2-((5-((1R,6S)-9-methyl-4-oxo-3,9-diazabicyclo[4.2.1]nonan-3-yl)pyridin-2-yl)amino)-7H-pyrrolo[2,3-d]pyrimidine-6-carboxamide , binimetinib, midostaurin, everolimus, 1-methyl-5-((2-(5-(trifluoromethyl)-1H-imidazol-2-yl)pyridin-4-yl)oxy)-N-(4-(trifluoromethyl)phenyl)-1H-benzo[d]imidazol-2-amine, pasireotide diaspartate, dovitinib, (R,E)-N-(7-chloro-1-(1-(4-(dimethylamino)but-2-enoyl)azepan-3-yl)-1H-benzo[d]imidazol-2-yl)-2-methylisonicotinamide, N6-(2-isopropoxy-1-methyl-4-(1-methylpiperidin-4-yl)phenyl)-N4-(2-(isopropyl sulfonyl)phenyl)-1H-pyrazolo[3,4-d]pyrimidine-4,6-diamine, 3-(4-(4-((5-chloro-4-((5-methyl-1H-pyrazol-3-yl)amino)pyrimidin-2-yl)amino)-5-fluoro-2-methylphenyl)piperidin-1-yl)thietane 1,1-dioxide, 5-chloro-N2-(2-fluoro-5-methyl-4-(1-(tetrahydro-2H-pyran-4-yl)piperidin-4-yl)phenyl)-N4-(5-methyl-1H-pyrazol-3-yl)pyrimidine-2,4-diamine, 5-chloro-N2-(4-(1-ethylpiperidin-4-yl)-2-fluoro-5-methylphenyl)-N4-(5-methyl-1H-pyrazol-3-yl)pyrimidine-2,4-diamine, valspodar, and vatalanib succinate.

In certain embodiments, the compounds of the present disclosure are administered in conjunction with one or more vaccines intended to stimulate an immune response to one or more predetermined antigens. Examples of target antigens that may find use in the disclosure include, but are not limited to, tumor antigens: mesothelin, Wilms' tumor-1 associated protein, including isoform A; isoform B, isoform C; isoform D, stratum corneum chymotryptic enzyme and variants et al, MHC class I chain-related protein A and MHC class I chain-related protein B, CCK-B, glypican-3, coactosin-like protein, prostate stem cell antigen, PAP, PSA, PSM, PSMA, STEAP, PCTA-1, PTI-1, prostase, proteinase 3, cancer testis antigens etc. This list is not meant to be limiting.

### Administration and Dosages

Administration of the compounds of the present disclosure may be affected by any method that enables delivery of the compounds to the site of action. These methods include a variety of means including, but are not limited to, non-parenterally, parenterally, inhalation spray, topically, or rectally in formulations containing pharmaceutically acceptable carriers, adjuvants and vehicles. “Non-parenteral administration” encompasses oral, buccal, sublingual, topical, transdermal, ophthalmic, otic, nasal, rectal, cervical, pulmonary, mucosal, and vaginal routes. The term parenteral as used here includes but is not limited to subcutaneous, intravenous, intramuscular, intraarterial, intradermal, intrathecal and epidural injections with a variety of infusion techniques. Intraarterial and intravenous injection as used herein includes administration through catheters. Administration via intracoronary stents and intracoronary reservoirs is also contemplated. Intra-tumoral (directly into the tumor mass) or peri-tumoral (around the tumor mass) administration of the compounds of the present disclosure may directly activate locally infiltrating DC, directly promote tumor cell apoptosis or sensitize tumor cells to cytotoxic agents.

In one embodiment, the compounds of the present disclosure may be administered orally. Oral administration may involve swallowing, so that the compound enters the gastrointestinal tract, or buccal or sublingual administration may be employed by which the compound enters the blood stream directly from the mouth.

The compounds of the present disclosure may also be administered directly into the blood stream, into muscle, into an internal organ or into a tumor. Suitable means for parenteral administration include intravenous, intraarterial, intraperitoneal, intrathecal, intraventricular, intraurethral, intrasternal, intracranial, intramuscular, intra-tumoral (directly into the tumor mass), peri-tumoral (around the tumor mass) and subcutaneous. Suitable devices for parenteral administration include needle (including microneedle) injectors, needle-free injectors and infusion techniques.

The dosages may be varied depending on the requirement of the patient, the severity of the condition being treating and the particular compound being employed. Determination of the proper dosage for a particular situation can be determined by one skilled in the medical arts. The total daily dosage may be divided and administered in portions throughout the day or by means providing continuous delivery.

In certain embodiments, the compounds described herein are administered at a dosage of from about 0.001 mg/Kg to about 500 mg/Kg (e.g., from about 0.001 mg/Kg to about 200 mg/Kg; from about 0.01 mg/Kg to about 200 mg/Kg; from about 0.01 mg/Kg to about 150 mg/Kg; from about 0.01 mg/Kg to about 100 mg/Kg; from about 0.01 mg/Kg to about 50 mg/Kg; from about 0.01 mg/Kg to about 10 mg/Kg; from about 0.01 mg/Kg to about 5 mg/Kg; from about 0.01 mg/Kg to about 1 mg/Kg; from about 0.01 mg/Kg to about 0.5 mg/Kg; from about 0.01 mg/Kg to about 0.1 mg/Kg; from about 0.1 mg/Kg to about 200 mg/Kg; from about 0.1 mg/Kg to about 150 mg/Kg; from about 0.1 mg/Kg to about 100 mg/Kg; from about 0.1 mg/Kg to about 50 mg/Kg; from about 0.1 mg/Kg to about 10 mg/Kg; from about 0.1 mg/Kg to about 5 mg/Kg; from about 0.1 mg/Kg to about 1 mg/Kg; from about 0.1 mg/Kg to about 0.5 mg/Kg).

The foregoing dosages can be administered on a daily basis (e.g., as a single dose or as two or more divided doses) or non-daily basis (e.g., every other day, every two days, every three days, once weekly, twice weeks, once every two weeks, once a month).

In certain embodiments, the period of administration of a compound described herein is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Click Chemistry

In certain embodiments the compounds, linkers, and formula disclosed herein comprise a functional group capable of reacting through click chemistry. As used herein, click chemistry refers to a 1,3-dipolar cycloaddition or [3+2] cycloaddition between an azide and an alkyne to form a 1,2,3-triazole. The terms “1,3-dipolar cycloaddition” or “[3+2] cycloaddition” also encompasses “copperless” 1,3-dipolar cycloadditions between azides and cyclooctynes.

Thus, unless stated otherwise, the description of any triazole compound herein is meant to include regioisomers of a compound, as well as mixtures thereof.

For example, the [3+2] cycloaddition of an azide and alkyne may produce two regioisomeric triazoles as follows:

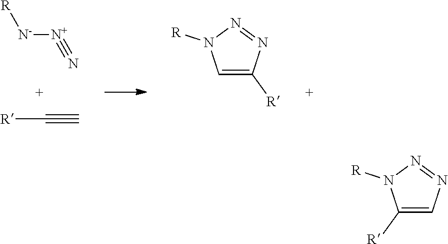

In certain embodiments, the alkyne is a strained cycloalkynyl or heterocycloalkynyl, and the cycloaddition reaction may be performed in the presence or absence of a catalyst. In certain embodiments, for example, the cycloaddition reaction may occur spontaneously by a reaction called strain-promoted azide-alkyne cycloaddition (SPAAC), which is known in the art as “metal-free click chemistry”. In certain embodiments, the strained cycloalkynyl or heterocycloalkynyl is as described herein.

Such catalyst-free [3+2] cycloadditions can be used in methods described herein to form conjugates of the present disclosure. Alkynes can be activated by ring strain such as, by way of example only, eight membered ring structures, appending electron-withdrawing groups to such alkyne rings, or alkynes can be activated by the addition of a Lewis acid such as, Au(1) or Au(lll). Alkynes activated by ring strain have been described. For example, the cyclooctynes and difluorocyclooctynes described by Agard et al., J. Am. Chem. Soc, 2004, 126 (46):15046-15047, the dibenzocyclooctynes described by Boon et al., WO2009/067663 A1 (2009), and the aza-dibenzocyclooctynes described by Debets et al., Chem. Comm., 2010, 46:97-99.

In certain embodiments conjugates of the present disclosure can be obtained by reacting an alkyne functionalized compound comprising therapeutic agent A¹ with an azide functionalized compound comprising therapeutic A², to form a conjugate, as described herein. In other embodiments the compound comprising therapeutic agent A² can possess an activated alkyne moiety, and the compound comprising therapeutic agent A¹ possesses an azide moiety.

In certain embodiments, an azide in a compound comprising a STING agonist reacts with the alkyne in a compound comprising a TLR9 agonist to form a triazole moiety (e.g. via a 1,3-dipolar cycloaddition). In certain embodiments, an azide in a compound comprising a TLR9 agonist reacts with the alkyne in a compound comprising a STING agonist to form a triazole moiety.

In certain embodiments, an azide in a compound comprising a TLR7/8 agonist reacts with an alkyne in a compound comprising a TLR9 agonist to form a triazole moiety (e.g. via a 1,3-dipolar cycloaddition). In certain embodiments, an azide in a compound comprising a TLR9 agonist reacts with an alkyne in a TLR7/8 agonist to form a triazole moiety (e.g. via a 1,3-dipolar cycloaddition). In certain embodiments, an azide in a compound comprising a TLR7/8 agonist reacts with an alkyne in a compound comprising a TLR7/8 agonist to form a triazole moiety (e.g. via a 1,3-dipolar cycloaddition).

In certain embodiments, an azide in a compound comprising a STING agonist reacts an alkyne in a compound comprising a TLR7/8 agonist to form a triazole moiety (e.g. via a 1,3-dipolar cycloaddition). In certain embodiments, an azide in a compound comprising a TLR7/8 agonist reacts with an alkyne in a compound comprising a STING agonist to form a triazole moiety (e.g. via a 1,3-dipolar cycloaddition).

In certain embodiments, upon conjugation, the triazole is represented by:

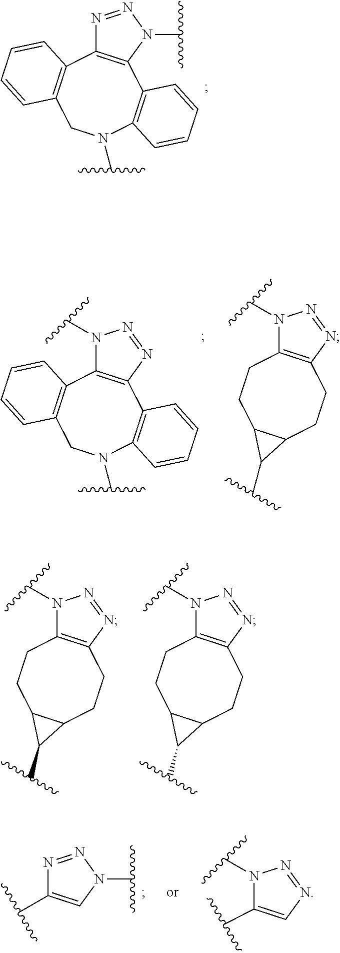

In certain embodiments of the compounds, conjugates, and formula disclosed herein, T is selected from:

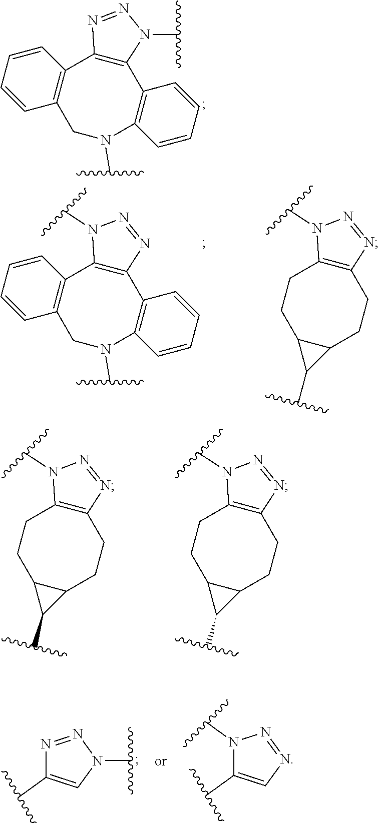

In certain embodiments, click chemistry product groups of the present disclosure comprise a triazole group.

In certain embodiments, click chemistry product groups are selected from the group consisting of:

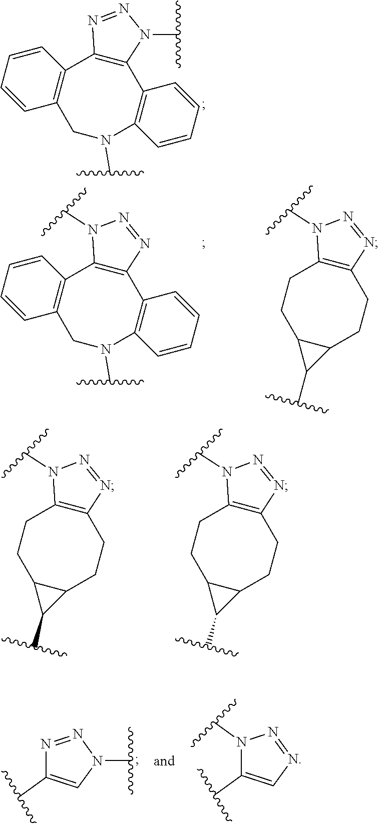

In certain embodiments of any compounds, conjugates, STING agonist derivatives, or TLR9 agonist derivatives of any one of formula disclosed herein comprising a triazole functional group (T), the triazole functional group can exist as a mixture of regioisomers resulting in the compounds, conjugates, STING agonist derivatives, or TLR9 agonist derivatives to exist as a mixture of regioisomers.

### Method of Making

Compounds of the disclosure and intermediates thereof can be prepared in a number of ways known to one of ordinary skill in the art of organic synthesis. Non-limiting examples are illustrated below. It is understood by one skilled in the art that these methods are representative and are not limiting. Starting materials and intermediates can be purchased from commercial sources or can be made from known procedures. The skilled artisan will also recognize that conditions and reagents described herein can be interchanged with alternative art-recognized equivalents. The variations of the examples provided below within the scope of the claims are within the purview of one skilled in the art and are considered to fall within the scope of the disclosure as described and claimed herein. The reader will recognize that the skilled artisan, provided with the present disclosure will be able to prepare and use the disclosure without exhaustive examples.

The depiction of the stereochemistry in the structures of the compound synthesized was determined to the best of knowledge at the time of synthesis. Due to the complexity of some of the compounds disclosed herein, the identification of the stereocenters are not meant to be absolute. The present disclosure includes compounds and conjugates specifically disclosed as depicted (drawn out) as well as actually synthesized according to the procedures described herein, regardless of whether the initial stereochemical center assignments were correct or not.

### Chiral Synthesis of 3, 3-Cyclic Nucleotide Boranophosphates

One example of synthetic route for CDN is outlined in Scheme 1. The 3-phosphoramidite of fully protected ribo-nucleoside or thio-nucleoside was reacted with 5′-hydroxyl of second modified ribo-nucleoside or thio-nucleoside to give a phosphite triester, which was then oxidized with dimethyl sulfide-borane. After removal of DMTr under acidic condition, the cyclization was achieved in the presence of (2R,3aS,6R,7aS)-3a-methyl-2-((perfluorophenyl)thio)-6-(prop-1-en-2-yl)hexahydrobenzo[d][1,3,2]oxathiaphosphole 2-sulfide or (2S,3aS,6R,7aS)-3a-methyl-2-((perfluorophenyl)thio)-6-(prop-1-en-2-yl)hexahydrobenzo[d][1,3,2]oxathiaphosphole 2-sulfide. Treatment with base such as methyl amine or ammonium provided the cyclic dinucleotides. This method, as well as other methods described herein, can be used to synthesize the CDNs of WO 2019/043634, which is hereby incorporated by reference in all its entirety.

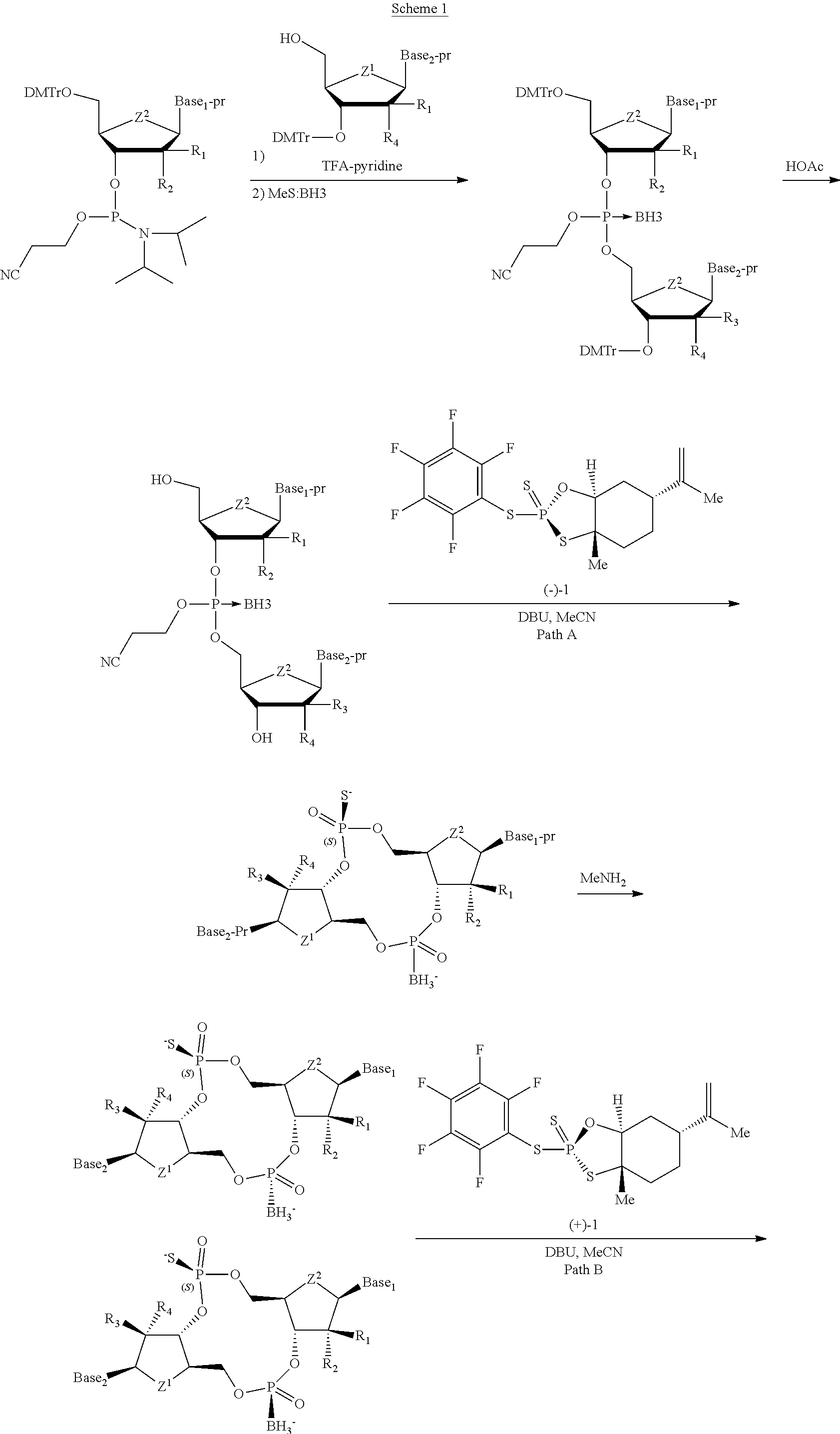

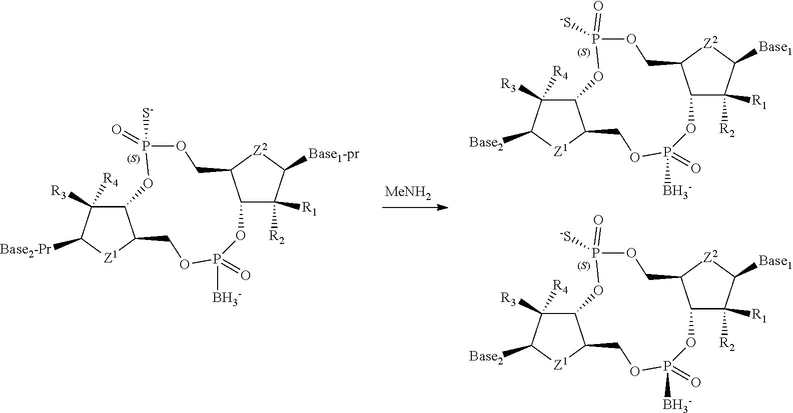

Base₁ or Base₂ is selected from nucleobases G, A, U, T, C and the analogues or derivatives of nucleobases. Base₁-Pr or Base₂-Pr represents protected nucleobases, the protection group can be benzoyl group or isobutyryl group. R₁, R₂, R₃ and R₄ are each independently H, F, OH or OTBS, Z¹ and Z² are each independently O or S.
Synthesis of STING Agonist with a Linker

A general method for preparing STING agonists with a linker is shown in Scheme 2.

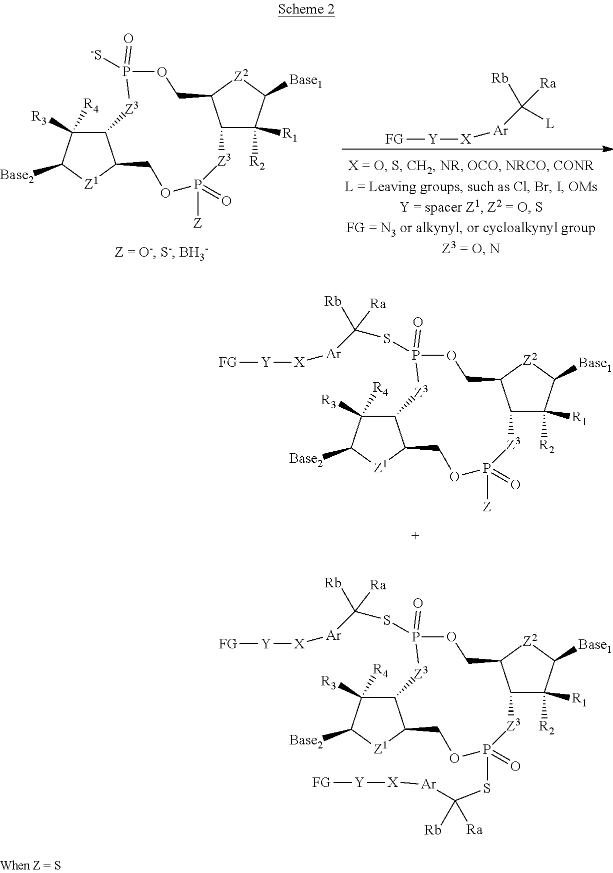

Base 1 and Base 2 can be connected together through a spacer between the amino group of two bases.

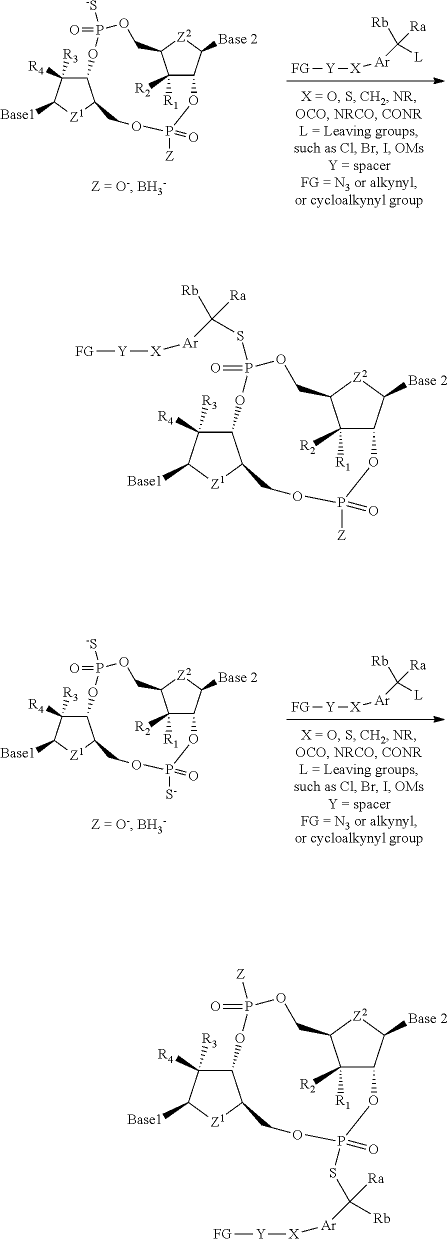

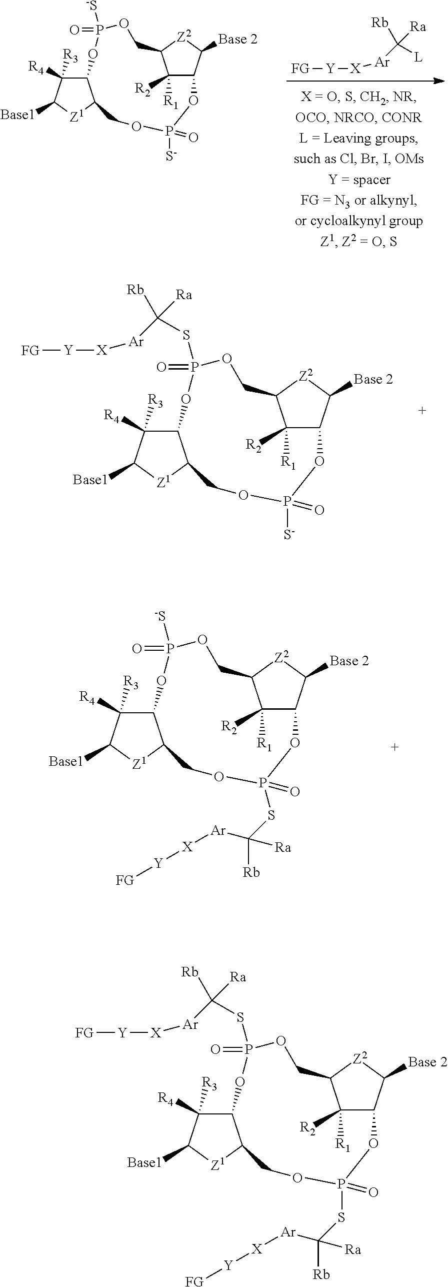

Ra and Rb are each independently hydrogen or a C₁-C₆ alkyl group.

Treatment of CDN salt with benzylic halide, benzylic mesylate or benzylic tosylate in a mixed organic solvent such as THF and acetone provided a cyclic dinucleotide appended with a linker. In the case of cyclic nucleotides bearing two phosphonothioates internucleotidic linkages, it is possible to provide CDN with two linkers.

Synthesis of Releasable Linker-Formula II and Non-Releasable Linker

An exemplary method for preparing releasable linker and non-releasable linker is outlined in Scheme 3. The reaction of carbonyl chloride, alkyl halide, mesylate or tosylate and S3-a2 in the presence of base (such as DMAP, Et₃N) gave the key intermediate S3-c. Alternatively, S3-c may be synthesized from the coupling reaction of carboxylic acid with S3-a1 followed by deprotection of hydroxy group. The conversion of benzylic hydroxy group to better leaving groups such as chloride, iodide, bromide, mesylate or tosylate is well-established in the literature. Carbonates S3-e and S3-f were obtained through the reaction of intermediate S3-c with N,N′-disuccinimidyl carbonate and 4-nitrophenyl carbonochloridate in the presence of base (such as DMAP or pyridine) respectively.

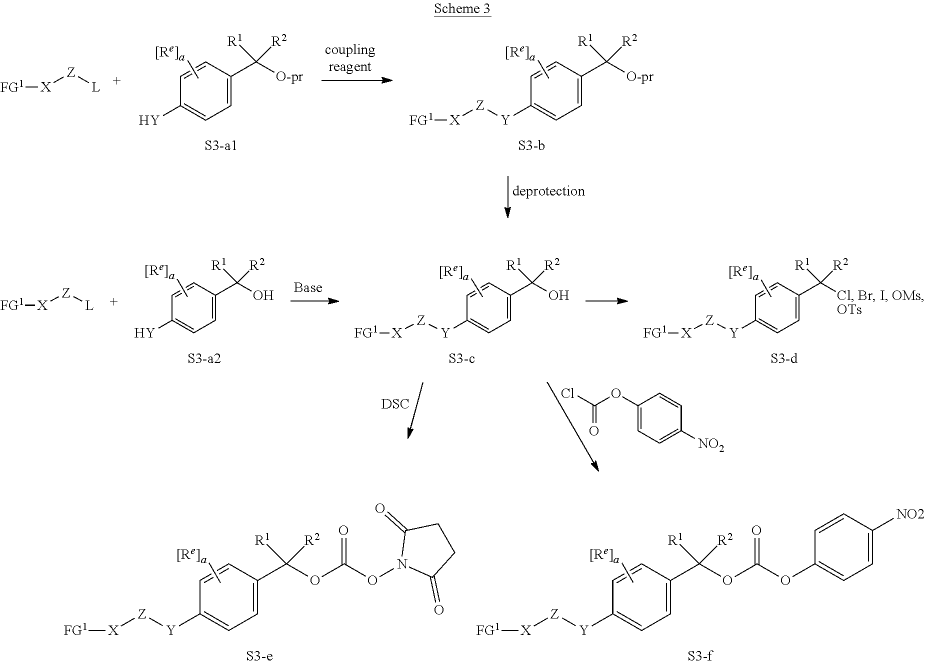

X=Spacer, Y═O, S, N, Z═CO, CH₂, When Z═CO, L=OH or Cl, when Z═CH₂, L is halide, mesylate or tosylate, FG¹ is a functional group capable of reacting through click chemistry. R¹ and R² are each independently hydrogen or a C₁-C₆ alkyl group, R^{e} is independently selected from the group consisting of nitro, cyano, halogen, substituted or unsubstituted amide, substituted or unsubstituted sulfone, substituted or unsubstituted sulfonamide, substituted or unsubstituted alkoxy, substituted or unsubstituted alkyl, substituted or unsubstituted alkenyl, substituted or unsubstituted alkynyl, substituted or unsubstituted cycloalkyl, substituted or unsubstituted aryl and substituted or unsubstituted heteroaryl.

There are several other methods to prepare releasable linker-formula II. As detailed in Scheme 4, the reaction of compound S3-a1 with cyclic anhydride gave key intermediate S3-g, which was then converted to S3-h through coupling reaction with amine or hydroxylamine. After deprotection, the benzylic hydroxy group was transformed to FG² following the well-known synthetic methods. Scheme 5 depicts a synthetic route starting from intermediate S3-j, which was synthesized by coupling reaction of carbonyl acid and S3-a1. Alkylation of S3-j followed by deprotection of hydroxy group presented S3-k, which was a building block for releasable linker S3-l.

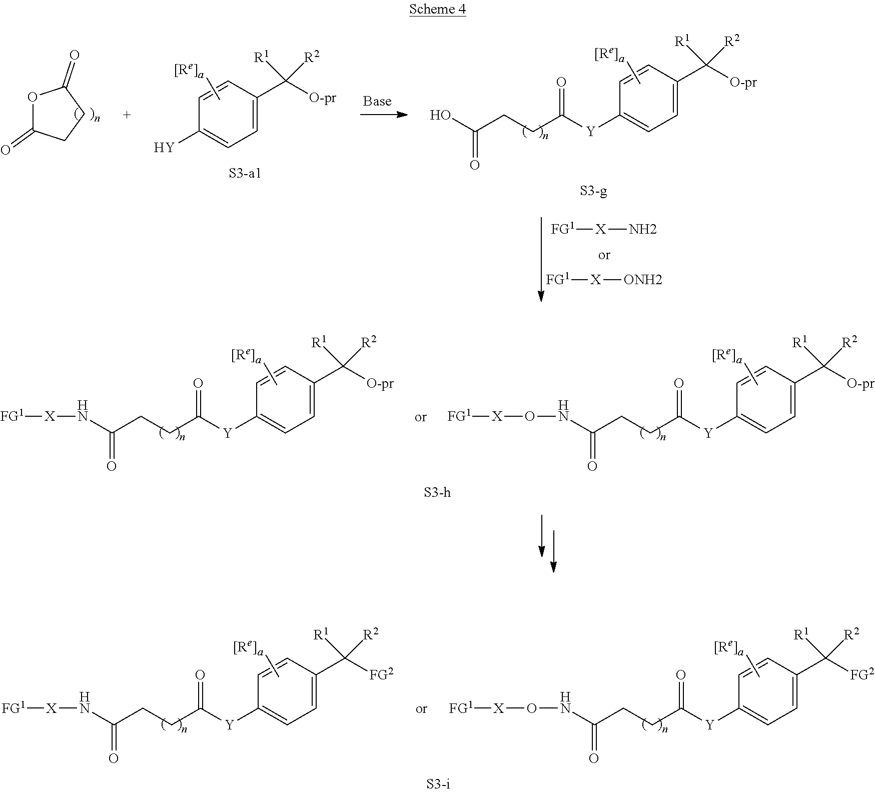

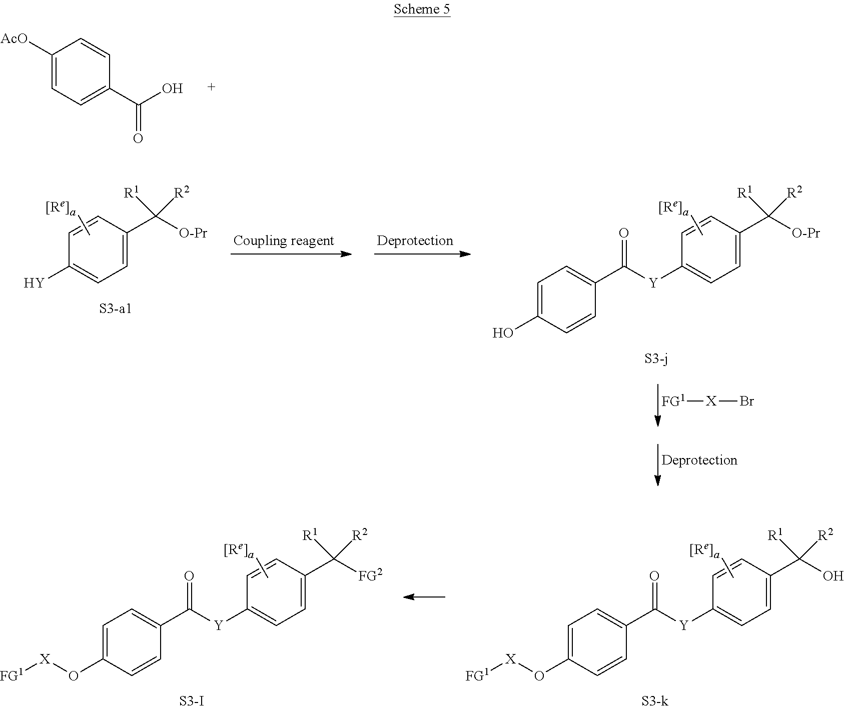

X=Spacer, Y═O, S, FG¹ is a functional group capable of reacting through click chemistry, FG² is a functional group selected from chloro, bromo, iodo, tosylate or mesylate functional groups;

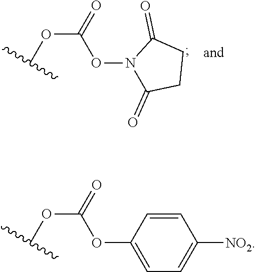 [SMILES: *OC(=O)ON1C(=O)CCC1=O ; *OC(=O)Oc1ccc([N+](=O)[O-])cc1]

R¹ and R² are each independently hydrogen or a C₁-C₆ alkyl group, R^{e} is independently selected from the group consisting of nitro, cyano, halogen, substituted or unsubstituted amide, substituted or unsubstituted sulfone, substituted or unsubstituted sulfonamide, substituted or unsubstituted alkoxy, substituted or unsubstituted alkyl, substituted or unsubstituted alkenyl, substituted or unsubstituted alkynyl, substituted or unsubstituted cycloalkyl, substituted or unsubstituted aryl and substituted or unsubstituted heteroaryl.

Synthesis of TLR7/8 Agonist with a Releasable Linker

One example of synthetic route for TLR7/8 agonist is outlined in Scheme 6. The reaction of compound S3-e or S3-f with 1-(4-amino-2-(ethoxymethyl)-1H-imidazo[4,5-c]quinolin-1-yl)-2-methylpropan-2-ol (R848), in the presence of base such as DIEA or pyridine by conventional heating or microwave irradiation, provided TLR7/8 agonist S6-a with a releasable linker.

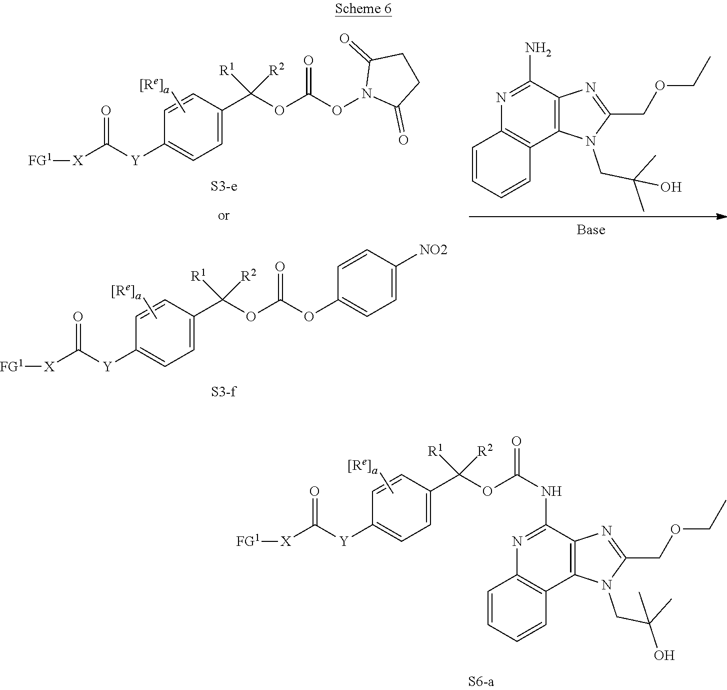

R¹ and R² are each independently hydrogen or a C₁-C₆ alkyl group, R^{e} is independently selected from the group consisting of nitro, cyano, halogen, substituted or unsubstituted amide, substituted or unsubstituted sulfone, substituted or unsubstituted sulfonamide, substituted or unsubstituted alkoxy, substituted or unsubstituted alkyl, substituted or unsubstituted alkenyl, substituted or unsubstituted alkynyl, substituted or unsubstituted cycloalkyl, substituted or unsubstituted aryl and substituted or unsubstituted heteroaryl, X=spacer, Y═O, S.

Synthesis of TLR9 Agonist with a Releasable Linker

Another example of synthetic route for TLR9 agonist with a releasable linker is outlined in Scheme 7. CpG-X¹—NH₂ is an oligodeoxynucleotide connecting with spacer at 3′ positions. The reaction of compound S3-e or S3-f with CpG-X¹—NH₂ provided TLR9 agonist S7-a with a releasable linker at 3′ position in the presence of base.

Similarly, compound S3-e or S3-f can react with NH₂-X²-CpG with spacer at 5′ position or NH₂-X²-CpG-X¹—NH₂ with spacer at both 5′ and 3′ positions to provide TLR9 agonist with a releasable linker at corresponding 5′ or both 5′ and 3′ positions.

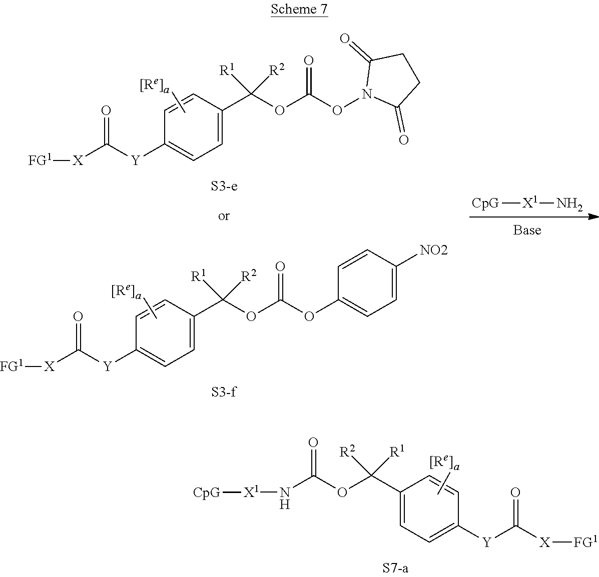

Synthesis of TLR9 Agonist with a Non-Releasable Linker

One example of synthetic route for TLR9 agonist with a non-releasable linker is outlined in Scheme 8. CpG-X¹—NH₂ is an oligodeoxynucleotide connecting with spacer at 3′ positions. The reaction of compound S8-a with CpG-X¹—NH₂ provided TLR9 agonist S8-b with a non-releasable linker at 3′ position in the presence of base (such as DMAP). Similarly, compound FG¹-X—COOH NHS ester can react with NH₂-X²-CpG with a spacer at 5′ position or NH₂-X²-CpG-X¹—NH₂ with spacers at both 5′ and 3′ positions to provide different TLR9 agonist with a non-releasable linker at corresponding 5′ or both 5′ and 3′ positions.

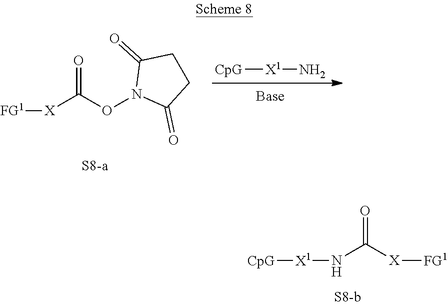

X and X¹ are spacers.

Synthesis of Final Conjugates

General methods for synthesis of conjugates are shown in Scheme 9.

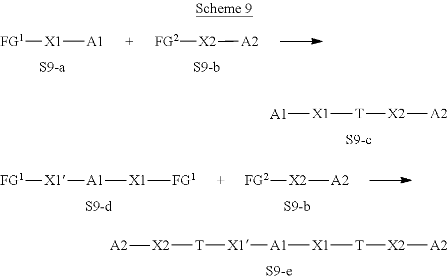

A¹ and A² are each independently a therapeutic agent or an active moiety of a therapeutic agent; or a compound that decomposes to a therapeutic agent, X1 and X2 are spacers, FG1 or FG2 is independently azido, alkynyl or cycloalkynyl, each T is independently a triazole functional group.

The click chemistry of S9-a or S9-d (azide or alkyne) with S9-b (independently alkyne or azide) was carried out in a mixed solvent of aqueous buffer (pH 7.4) and organic solvent (DMSO or DMF) to provide the final conjugate S9-c or S9-e respectively, in the presence of copper catalyst (for alkyne) or in the absence of copper catalyst (for cyclic alkyne).

Abbreviations: DCA=dichloroacetic acid. DCM=CH₂Cl₂=dichloromethane. OCE=OCH₂CH₂CN. ACN=acetonitrile. MeOH=methanol. NH₄OAc=ammonium acetate. TBS=tert-butyldimethylsilyl. TMSCl=trimethylsilyl chloride. THF=tetrahydrofuran. DMTr=4,4′-dimethoxytrityl. DMTrCl=4,4′-dimethoxytrityl chloride. DMSO=dimethylsulfoxide. DMF=dimethylformamide. THF=tetrahydrofuran. TBDPSCl=tert-butyl(chloro)diphenylsilane. DCC=N,N′-dicyclohexylcarbodiimide. HATU=hexafluorophosphate azabenzotriazole tetramethyl uranium. EDCI=1-ethyl-3-(3-dimethylaminopropyl)carbodiimide. DMAP=4-dimethylaminopyridine. DBU=1,8-Diazabicyclo[5.4. 0]undec-7-ene. EDTA=ethylenediaminetetraacetic acid. DIEA=diisopropylethyl amine. TEAB=triethylammonium bicarbonate. TBME=tert-butyl methyl ether. MsCl=methyl chlorosulfate.

### PREPARATION OF INTERMEDIATES

### Example I-1 Synthesis of 4-(iodomethyl) phenyl 4-azidobutanoate

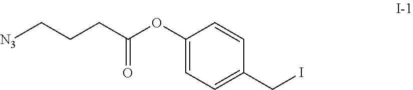 [SMILES: [N-]=[N+]=NCCCC(=O)Oc1ccc(CI)cc1]

### Step 1. Synthesis of 4-(((tert-butyldiphenylsilyl)oxy)methyl)phenol I-1b

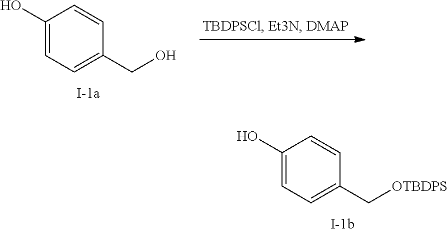

Into a 250-mL round-bottom flask, TBDPSCl (1.10 g, 4.002 mmol, 1.0 eq) was added dropwise to a solution of 4-(hydroxymethyl)phenol (500.00 mg, 4.028 mmol, 1.00 eq), Et₃N (444.00 mg, 4.388 mmol, 1.1 eq) and DMAP (48.80 mg, 0.399 mmol, 0.10 eq) in DCM (20.00 mL). The solution was stirred for 2 hours at room temperature. The reaction was then quenched by the addition of 30 mL of NaHCO3(sat) at 0° C. and extracted with 4×30 mL of ethyl acetate, the combined organic layers were combined and concentrated. The residue was purified by chromatography to give 800 mg (54.79%) of 4-[[(tert-butyldiphenylsilyl)oxy]methyl]phenol as an oil.

### Step 2. Synthesis of 4-(((tert-butyldiphenylsilyl)oxy)methyl)phenyl 4-azidobutanoate I-1c

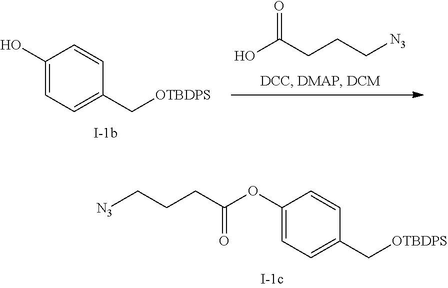

The mixture of 4-[[(tert-butyldiphenylsilyl)oxy]methyl]phenol (1000.00 mg, 2.758 mmol, 1.00 eq), 4-azidobutanoic acid (299.16 mg, 2.317 mmol, 0.84 eq), DCC (711.39 mg, 3.448 mmol, 1.25 eq) and DMAP (28.64 mg, 0.234 mmol, 0.08 eq) in DCM (10 mL) was stirred for 2 hours at room temperature. The reaction was quenched by the addition of 100 mL of NaHCO₃ (sat.) at 0° C. and extracted with 3×100 mL of ethyl acetate, the combined organic layers were dried over anhydrous sodium sulfate. After filtration, the filtrate was concentrated to give a residue, which was purified by Flash-Prep-HPLC to give 4-[[(tert-butyldiphenylsilyl)oxy]methyl]phenyl 4-azidobutanoate (600 mg, 45.93%).

### Step 3. Synthesis of 4-(hydroxymethyl) phenyl 4-azidobutanoate I-1d

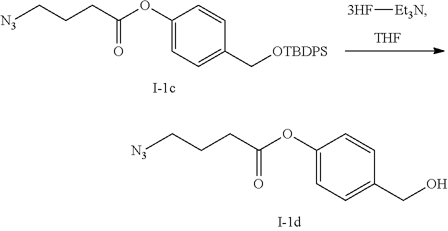

Et₃N·3HF (12.81 g, 79.462 mmol, 25.0 eq) was added to a solution of 4-[[(tert-butyldiphenylsilyl)oxy]methyl]phenyl 4-azidobutanoate (1.50 g, 3.167 mmol, 1.00 eq) and Et₃N (31.95 g, 315.743 mmol, 99.70 eq) in THF (40.00 mL) at 0° C. The resulting solution was stirred overnight at room temperature. The reaction was then quenched by the addition of 60 mL of NaHCO₃ (sat.) at 0° C. and extracted with 4×60 mL of dichloromethane, the organic layers were combined and concentrated to give a residue, which was purified by Flash-Prep-HPLC to give 580 mg (77.85%) of 4-(hydroxymethyl) phenyl 4-azidobutanoate as oil. ¹H NMR (300 MHz, DMSO-d6) δ 7.42-7.25 (m, 2H), 7.13-6.94 (m, 2H), 5.20 (t, J=5.7 Hz, 1H), 4.48 (d, J=5.5 Hz, 2H), 3.43 (t, J=6.8 Hz, 2H), 2.64 (t, J=7.3 Hz, 2H), 1.88 (p, J=7.0 Hz, 2H).

### Step 4. Synthesis of 4-(iodomethyl) phenyl 4-azidobutanoate I-1

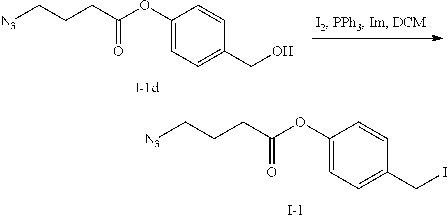 [SMILES: [N-]=[N+]=NCCCC(=O)Oc1ccc(CO)cc1 ; [N-]=[N+]=NCCCC(=O)Oc1ccc(CI)cc1 ; [I-]]

Iodine (107.00 mg, 0.422 mmol, 2.0 eq) was added to a solution of 4-(hydroxymethyl) phenyl 4-azidobutanoate (50.00 mg, 0.213 mmol, 1.00 eq), imidazole (32.00 mg, 0.470 mmol, 2.2 eq) and PPh₃ (67.00 mg, 0.255 mmol, 1.20 eq) in DCM (2.00 mL), the resulting solution was stirred for 30 min at 0° C. and concentrated to give a residue, which was purified by chromatography (ethyl acetate/petroleum ether=1/1) to provide 30 mg (40.90%) of 4-(iodomethyl) phenyl 4-azidobutanoate as light yellow oil.

¹H NMR (300 MHz, Acetonitrile-d3) δ 7.56-7.36 (m, 2H), 7.20-6.93 (m, 2H), 4.57 (s, 2H), 3.43 (t, J=6.7 Hz, 2H), 2.64 (t, J=7.3 Hz, 2H), 1.95 (d, J=2.2 Hz, 2H).

### Example I-2 Synthesis of 4-(iodomethyl)phenyl 4-(2-azidoethoxy)benzoate I-2

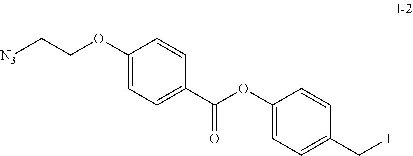 [SMILES: [N-]=[N+]=NCCOc1ccc(C(=O)Oc2ccc(CI)cc2)cc1]

### Step 1. Synthesis of 4-formylphenyl 4-acetoxybenzoate I-2b

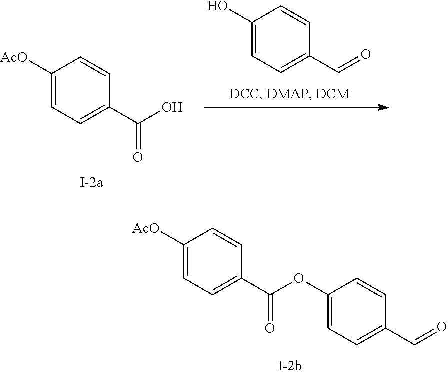 [SMILES: CC(=O)Oc1ccc(C(=O)O)cc1 ; O=Cc1ccc(O)cc1 ; CC(=O)Oc1ccc(C(=O)Oc2ccc(C=O)cc2)cc1]

The mixture of 4-acetoxybenzoic acid (5.00 g, 27.753 mmol, 1.00 eq), P-hydroxybenzaldehyde (4.07 g, 33.30 mmol, 1.20 eq), dicyclohexylcarbodiimide (8.59 g, 41.63 mmol, 1.50 eq) and DMAP (0.34 g, 2.775 mmol, 0.10 eq) in DCM (20.00 mL) was stirred overnight at 25° C. The mixture was then quenched by the addition of 1000 mL of ice water and extracted with 3×1 L of ethyl acetate, the organic layers were combined and dried over anhydrous sodium sulfate. Filtered, the filtrate was concentrated to give a residue, after purification by chromatography eluting with ethyl acetate/petroleum ether (1/5), 4 g (50.70%) of 4-formylphenyl 4-(acetyloxy)benzoate was obtained.

¹H NMR (400 MHz, DMSO-d6) δ 10.05 (s, 1H), 8.24-8.19 (m, 2H), 8.05 (d, J=8.6 Hz, 2H), 7.57 (d, J=8.2 Hz, 2H), 7.41 (d, J=8.2 Hz, 2H), 2.34 (s, 3H).

### Step 2. Synthesis of 4-(hydroxymethyl)phenyl 4-acetoxybenzoate I-2c

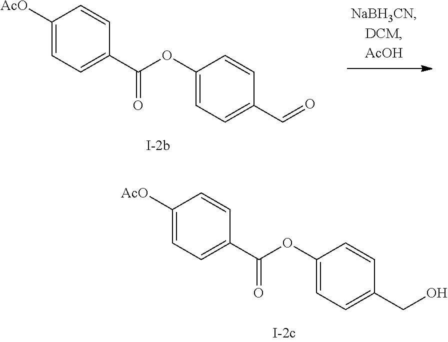 [SMILES: CC(=O)Oc1ccc(C(=O)Oc2ccc(C=O)cc2)cc1 ; CC(=O)Oc1ccc(C(=O)Oc2ccc(CO)cc2)cc1]

To a solution of 4-formylphenyl 4-(acetyloxy) benzoate (30.00 g, 105.535 mmol, 1.00 eq) in a mixed solvent of DCM (240.00 mL) and acetic acid (60.00 mL), was added NaBH₃CN (5.37 g, 85.483 mmol, 0.80 eq). The resulting solution was stirred for 20 min at 0° C. and additional 40 min at room temperature, quenched by the addition of 1000 mL of ice water and extracted with 3×1 L of ethyl acetate. The organic layers were combined and dried over anhydrous sodium sulfate, filtered. the filtrate was concentrated to give 28 g (92.68%) of 4-give (hydroxymethyl) phenyl 4-(acetyloxy)benzoate as a white solid, the crude product was used directly in the next step.

¹H NMR (300 MHz, Chloroform-d) δ 8.31-8.16 (m, 2H), 7.53-7.38 (m, 2H), 7.29-7.19 (m, 4H), 4.73 (s, 2H), 2.36 (s, 3H).

### Step 3. Synthesis of 4-(((tert-butyldiphenylsilyl)oxy)methyl)phenyl 4-acetoxybenzoate I-2d

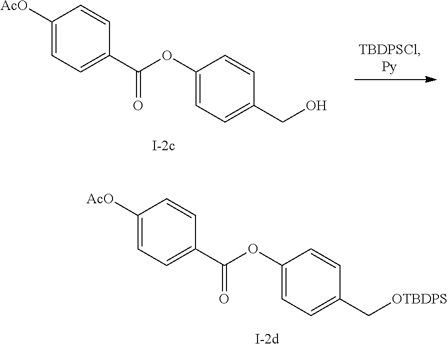

To a solution of 4-(hydroxymethyl) phenyl 4-(acetyloxy)benzoate (27 g, 1.00 eq) in pyridine (25.00 mL), was added TBDPSCl (48.00 mL, 2.00 eq),

the resulting mixture was stirred overnight at room temperature, quenched by the addition of 1000 mL of ice water and extracted with 3×1000 mL of ethyl acetate, the organic layer were dried over anhydrous sodium sulfate, filtered, the filtrate was concentrated and purified by chromatography with ethyl acetate/petroleum ether (1/5) to give 47.52 g (96.06%) of 4-[[(tert-butyldiphenylsilyl)oxy]methyl]phenyl 4-(acetyloxy)benzoate as a yellow oil.

LC-MS [M+H]+ 525.2

1H NMR (300 MHz, Chloroform-d) δ 8.30-8.22 (m, 2H), 7.76-7.66 (m, 4H), 7.48-7.33 (m, 7H), 7.30-7.23 (m, 3H), 7.18 (d, J=8.5 Hz, 2H), 4.80 (s, 2H), 2.06 (s, 3H), 1.12 (s, 9H).

### Step 4. Synthesis of 4-(((tert-butyldiphenylsilyl)oxy)methyl)phenyl 4-hydroxybenzoate I-2e

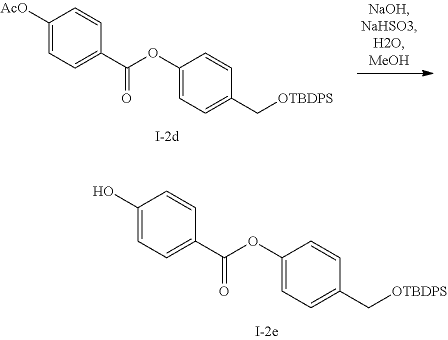

To a solution of 4-[[(tert-butyldiphenylsilyl)oxy] methyl] phenyl 4-(acetyloxy)benzoate (48 g, 1.00 eq) in MeOH (500.00 mL), was added a solution of 2.88 g of NaOH and 6 mg of NaHSO3 in water (120.00 mL) dropwise at room temperature and stirred for 30 min, quenched by the addition of 1000 mL of ice water and extracted with 3×1000 mL of ethyl acetate, the combined organic layers was dried over anhydrous sodium sulfate, filtered, the filtrate was concentrated and purified by chromatography eluting with ethyl acetate/petroleum ether (4/1) to give 36 g (81.82%) of 4-(((tert-butyldiphenylsilyl)oxy)methyl)phenyl 4-hydroxybenzoate.

LC-MS [M+H]⁺ 483.25. ¹H NMR (300 MHz, DMSO-d6) δ 10.51 (s, 1H), 8.03-7.92 (m, 2H), 7.66 (dt, J=7.6, 2.2 Hz, 4H), 7.49-7.38 (m, 8H), 7.21 (d, J=8.5 Hz, 2H), 6.97-6.88 (m, 2H), 4.80 (s, 2H), 1.05 (s, 9H).

### Step 5. Synthesis of 4-(((tert-butyldiphenylsilyl)oxy)methyl)phenyl 4-(2-hydroxyethoxy)benzoate

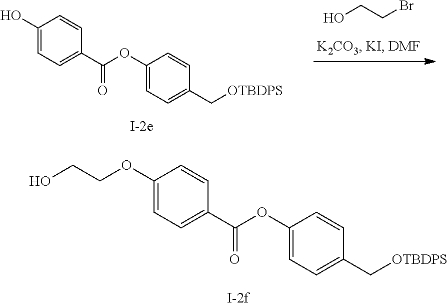

The mixture of 4-[[(tert-butyldiphenylsilyl)oxy]methyl]phenyl 4-hydroxybenzoate (35.00 g, 1.00 eq), 2-bromoethanol (45.309 g, Seq.), K₂CO₃ (50.110 g, 5 eq.) and KI (6.018 g, 5.00 eq) in DMF (500.00 mL) was stirred for 40 mins at 100° C., quenched by the addition of 1000 mL of ice water and extracted with 3×1000 mL of ethyl acetate, the combined organic layers were dried over anhydrous sodium sulfate, filtered, the filtrate was concentrated and purified by chromatography eluting with ethyl acetate/petroleum ether (1/1) to give 35 g (91.67%) of 4-[[(tert-butyldiphenylsilyl)oxy]methyl]phenyl 4-(2-hydroxyethoxy)benzoate as an oil.

LC-MS [M+23]⁺ 549.26

### Step 6. Synthesis of 4-(((tert-butyldiphenylsilyl)oxy)methyl)phenyl 4-(2-((methylsulfonyl)oxy)ethoxy)benzoate I-2g

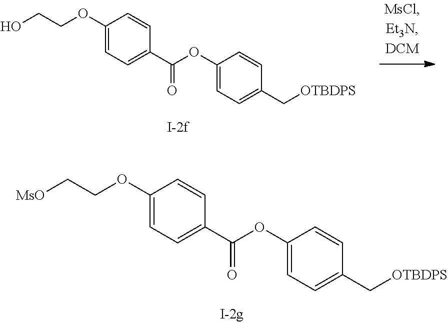

To a solution of 4-[[(tert-butyldiphenylsilyl)oxy]methyl]phenyl 4-(2-hydroxyethoxy)benzoate (1.5 g, 1.00 eq) and DCM (15.0 mL), was added Et₃N (578.036 mg, 2 eq) followed by MsCl (489 mg, 1.5 eq) dropwise at 0° C., the resulting solution was stirred at room temperature, quenched by the addition of 100 mL of ice water and extracted with 3×100 mL of ethyl acetate, the combined organic layers were dried over anhydrous sodium sulfate, filtered, the filtrate was concentrated and purified by chromatography eluting with ethyl acetate/petroleum ether (3/2) to give 1.6 g (92.89%) of 4-[[(tert-butyldiphenylsilyl) oxy]methyl]phenyl 4-[2-(methanesulfonyloxy)ethoxy]benzoate.

LC-MS [M+H]⁺ 605.25

¹H NMR (300 MHz, Acetonitrile-d3) δ 8.20-8.11 (m, 2H), 7.78-7.69 (m, 4H), 7.51-7.39 (m, 8H), 7.21 (d, J=8.5 Hz, 2H), 7.16-6.99 (m, 2H), 4.84 (s, 2H), 4.62-4.54 (m, 2H), 4.43-4.34 (m, 2H), 2.14 (s, 3H), 1.10 (s, 9H).

### Step 7. Synthesis of 4-(((tert-butyldiphenylsilyl)oxy)methyl)phenyl 4-(2-azidoethoxy)benzoate I-2h

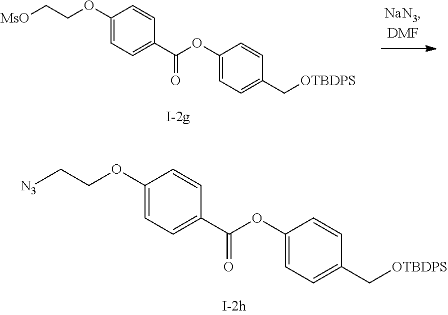

To a solution of 4-[[(tert-butyldiphenylsilyl)oxy]methyl]phenyl 4-[2-(methanesulfonyloxy)ethoxy]benzoate (1.6 g, 1.00 eq) in DMF (20.0 mL), was added NaN₃ (258 mg, 1.5 eq), the resulting solution was stirred for 1 h at 70° C., quenched by the addition of 100 mL of ice water and extracted with 3×100 mL of ethyl acetate, the combined organic layer were dried over anhydrous sodium sulfate, filtered, the filtrate was concentrated and purified by chromatography to give 1.4 g (95.96%) of 4-[[(tert-butyldiphenylsilyl)oxy]methyl]phenyl 4-(2-azidoethoxy)benzoate as an oil, the crude product was used directly in the next step. LC-MS [M+23]⁺ 574.25

### Step 8. Synthesis of 4-(hydroxymethyl)phenyl 4-(2-azidoethoxy)benzoate I-21

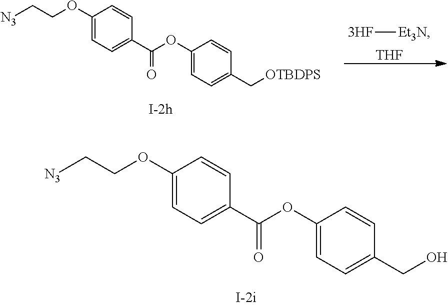

To a solution of 4-[[(tert-butyldiphenylsilyl)oxy]methyl]phenyl 4-(2-azidoethoxy) benzoate (1.40 g, 2.538 mmol, 1.00 eq) in THF (20.00 mL), was added 3HF-Et₃N (2.45 g, 15.228 mmol, 6.0 eq) dropwise at 0° C. The resulting solution was stirred overnight at room temperature, quenched by the addition of 100 mL of NaHCO₃ (sat.) at 0° C. and extracted with 3×100 mL of ethyl acetate, the combined organic layers were dried over anhydrous sodium sulfate, filtered, the filtrate was concentrated and purified by Flash-Prep-HPLC eluting with CH₃CN in 5-95% water to give 600 mg of (75.47%) of 4-(hydroxymethyl)phenyl 4-(2-azidoethoxy)benzoate. ¹H NMR (300 MHz, DMSO-d6) δ 8.09 (d, J=8.8 Hz, 2H), 7.38 (d, J=8.3 Hz, 2H), 7.17 (dd, J=15.0, 8.6 Hz, 4H), 4.56-4.45 (m, 2H), 4.30 (t, J=4.8 Hz, 2H), 3.71 (t, J=4.7 Hz, 2H).

### Step 9. Synthesis of 4-(iodomethyl)phenyl 4-(2-azidoethoxy)benzoate I-2

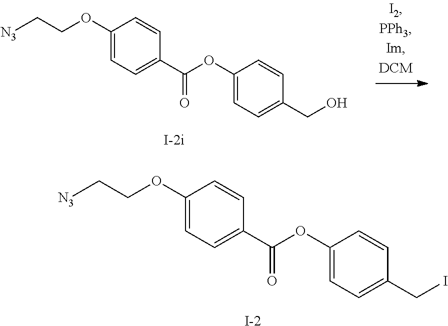

To a solution of 4-(hydroxymethyl)phenyl 4-(2-azidoethoxy)benzoate (100.00 mg, 1.00 eq) in DCM (1.0 mL), was added I₂ (97.21 mg, 1.2 eq), PPh₃ (100.46 mg, 1.2 eq) and imidazole (32.59 mg, 1.5 eq), the mixture was stirred for 20 min at 0° C., quenched by the addition of 30 mL of ice water and extracted with 3×30 mL of ethyl acetate. The combined organic layer were dried over anhydrous sodium sulfate, filtered, the filtrate was concentrated and purified to provide 90 mg (66.63%) of 4-(iodomethyl)phenyl 4-(2-azidoethoxy)benzoate.

LC-MS [M+H]⁺ 424.00

### Example I-3 Synthesis of 4-(hydroxymethyl)phenyl 4-[[(3-[2-azatricyclo[10.4.0.0\hat{ }[4,9]]hexadeca-1(12),4,6,8,13,15-hexaen-10-yn-2-yl]-3-oxopropyl)carbamoyl]methoxy]benzoate I-3

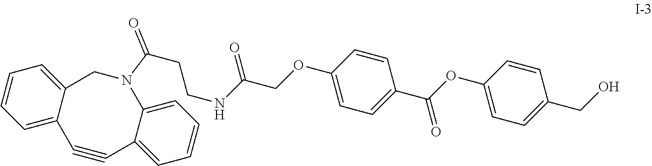 [SMILES: O=C(COc1ccc(C(=O)Oc2ccc(CO)cc2)cc1)NCCC(=O)N1Cc2ccccc2C#Cc2ccccc21]

### Step 1. Synthesis of N-(3-[2-azatricyclo[10.4.0.0\hat{ }[4,9]]hexadeca-1(12),4,6,8,13,15-hexaen-10-yn-2-yl]-3-oxopropyl)-2-bromoacetamide I-3b

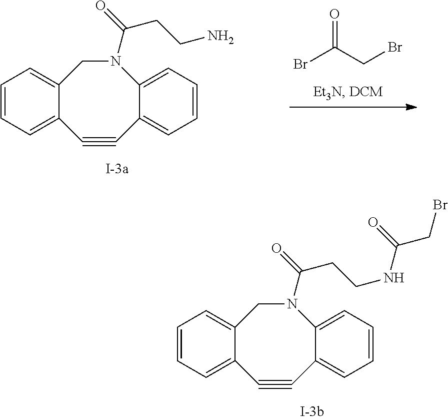 [SMILES: NCCC(=O)N1Cc2ccccc2C#Cc2ccccc21 ; O=C(Br)CBr ; O=C(CBr)NCCC(=O)N1Cc2ccccc2C#Cc2ccccc21]

Under nitrogen atmosphere, to a solution of 3-amino-1-[2-azatricyclo[10.4.0.0\hat{ }[4,9]]hexadeca-1(12),4,6,8,13,15-hexaen-10-yn-2-yl]propan-1-one (50.00 mg, 0.181 mmol, 1.00 eq) and Et₃N (27.46 mg, 0.271 mmol, 1.5 eq) in DCM (2.00 mL), was added bromoacetyl bromide (43.83 mg, 0.217 mmol, 1.2 eq) dropwise. The resulting solution was stirred for 30 min at room temperature. The reaction was then quenched by the addition of 10 mL of water and extracted with 3×10 mL of ethyl acetate, the combined organic layers were dried over anhydrous sodium sulfate, filtered and concentrated to give 50 mg (crude) of N-(3-[2-azatricyclo[10.4.0.0\hat{ }[4,9]]hexadeca-1(12),4,6,8,13,15-hexaen-10-yn-2-yl]-3-oxopropyl)-2-bromoacetamide.

### Step 2. Synthesis of 4-[[(tert-butyldiphenylsilyl)oxy]methyl]phenyl 4-[[(3-[2-azatricyclo[10.4.0.0\hat{ }[4,9]]hexadeca-1(16),4,6,8,12,14-hexaen-10-yn-2-yl]-3-oxopropyl)carbamoyl]methoxy]benzoate I-3c

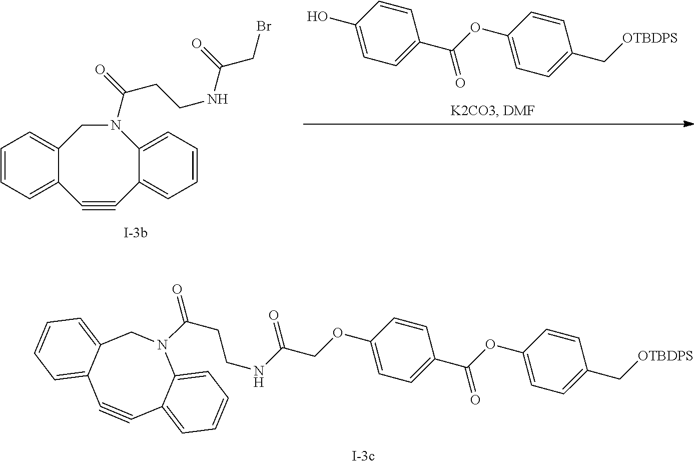

Into a 100-mL round-bottom flask purged and maintained with an inert atmosphere of argon, the mixture of N-(3-[2-azatricyclo[10.4.0.0\hat{ }[4,9]]hexadeca-1(16),4,6,8,12,14-hexaen-10-yn-2-yl]-3-oxopropyl)-2-bromoacetamide (1.40 g, 3.524 mmol, 1.00 eq), 4-[[(tert-butyldiphenylsilyl)oxy]methyl]phenyl 4-hydroxybenzoate (2.04 g, 4.229 mmol, 1.20 eq) and K₂CO₃ (1.46 g, 10.572 mmol, 3.00 eq) in DMF (10.00 mL) was stirred for 2 hours at room temperature. The reaction was then quenched by 50 mL of water and extracted with 3×100 mL of ethyl acetate, the combined organic layers were dried over anhydrous sodium sulfate, filtered and concentrated. The crude product was purified by Flash-Prep-HPLC eluting with ACN in 10-90% water to give 2.3 g (81.68%) of 4-[[(tert-butyldiphenylsilyl)oxy]methyl]phenyl 4-[[(3-[2-azatricyclo[10.4.0.0\hat{ }[4,9]]hexadeca-1(16),4,6,8,12,14-hexaen-10-yn-2-yl]-3-oxopropyl)carbamoyl]methoxy]benzoate.

### Step 3. Synthesis of 4-(hydroxymethyl)phenyl 4-[[(3-[2-azatricyclo[10.4.0.0\hat{ }[4,9]]hexadeca-1(12),4,6,8,13,15-hexaen-10-yn-2-yl]-3-oxopropyl)carbamoyl]methoxy]benzoate I-3

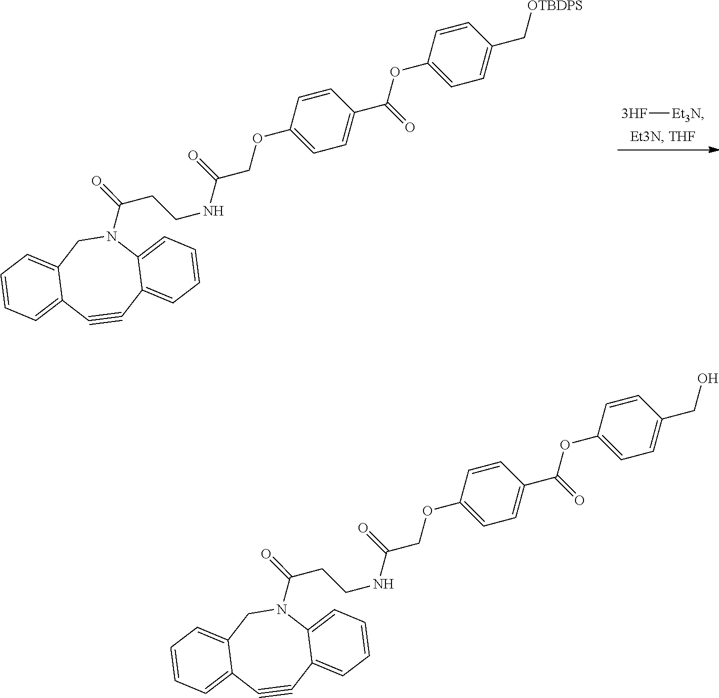

Into a 50-mL round-bottom flask purged and maintained with an inert atmosphere of argon, the mixture of 4-[[(tert-butyldiphenylsilyl)oxy]methyl]phenyl 4-[[(3-[2-azatricyclo[10.4.0.0\hat{ }[4,9]]hexadeca-1(12),4,6,8,13,15-hexaen-10-yn-2-yl]-3-oxopropyl)carbamoyl]methoxy]benzoate (200.00 mg, 0.250 mmol, 1.00 eq), Et₃N (151.97 mg, 1.502 mmol, 6.00 eq) and 3HF-Et₃N (241.80 mg, 1.502 mmol, 6.00 eq) in THF (5.00 mL) was stirred overnight at room temperature. The reaction was then quenched by the addition of 10 mL of NaHCO₃(aq) and extracted with 3×50 mL of ethyl acetate, the combined organic layers were dried over anhydrous sodium sulfate, filtered and concentrated. The crude product was purified by Flash-Prep-HPLC eluting with ACN in 10-90% water to provide 83.9 mg (59.79%) of 4-(hydroxymethyl)phenyl 4-[[(3-[2-azatricyclo[10.4.0.0\hat{ }[4,9]]hexadeca-1(12),4,6,8,13,15-hexaen-10-yn-2-yl]-3-oxopropyl)carbamoyl]methoxy]benzoate LC-MS (ES, m/z): 561.25 [M+H]⁺; ¹HNMR (300 MHz, DMSO-d6) δ 8.05 (t, J=10.0 Hz, 2H), 7.64 (dd, J=13.7, 6.3 Hz, 2H), 7.55-7.26 (m, 8H), 7.21 (d, J=8.1 Hz, 2H), 7.06 (d, J=8.5 Hz, 2H), 5.28 (s, 1H), 5.07 (d, J=14.1 Hz, 1H), 4.52 (d, J=9.6 Hz, 4H), 3.66 (d, J=14.0 Hz, 1H), 3.25 (s, 1H), 3.05 (s, 1H), 2.52 (m, 1H), 1.89 (dt, J=15.2, 7.0 Hz, 1H).

### Example I-4 Synthesis of 4-(hydroxymethyl)phenyl 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoate I-4

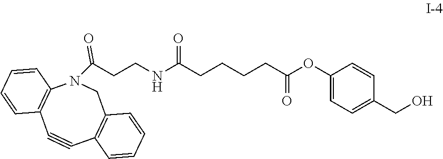 [SMILES: O=C(CCCCC(=O)Oc1ccc(CO)cc1)NCCC(=O)N1Cc2ccccc2C#Cc2ccccc21]

### Step 1. Synthesis of 6-(4-[[(tert-butyldiphenylsilyl)oxy]methyl]phenoxy)-6-oxohexanoic Acid I-4b

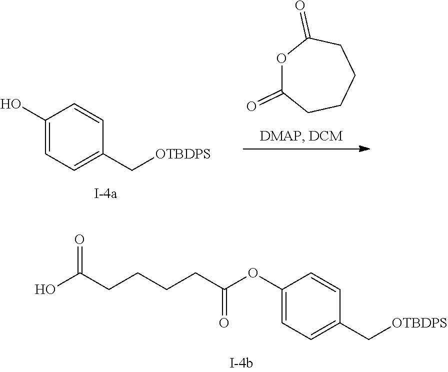

The mixture of 4-[[(tert-butyldiphenylsilyl)oxy]methyl]phenol (4.00 g, 11.033 mmol, 1.00 eq), DMAP (0.67 g, 5.517 mmol, 0.5 eq) and oxepane-2,7-dione (2.83 g, 22.066 mmol, 2.00 eq) in DCM (80.00 mL) was stirred overnight at room temperature. The reaction was then quenched by 100 mL of water. The pH value of the solution was adjusted to 5 with HCl and extracted with 3×80 mL of dichloromethane. The combined organic layers were dried, filtered and concentrated. The resulting residue was purified by Flash-Prep-HPLC eluting with ACN in 5-95% water to give 2.4 g (44.33%) of 6-(4-[[(tert-butyldiphenylsilyl)oxy]methyl]phenoxy)-6-oxohexanoic acid.

### Step 2. Synthesis of 4-(((tert-butyldiphenylsilyl)oxy)methyl)phenyl 6-chloro-6-oxohexanoate I-4c

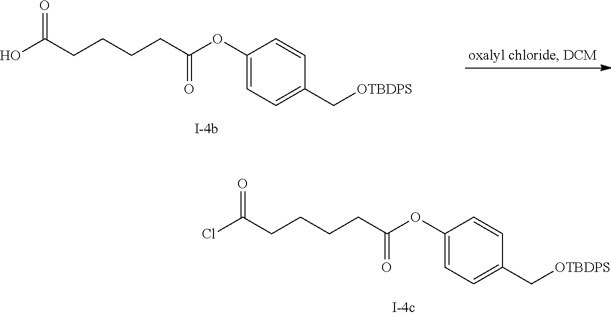

Under atmosphere of argon, to a solution of placed 6-(4-[[(tert-butyldiphenylsilyl)oxy]methyl]phenoxy)-6-oxohexanoic acid (550.00 mg, 1.121 mmol, 1.00 equiv) in DCM (11.00 mL, 129.518 mmol, 154.37 eq), was added DMF (cat) followed by oxalyl chloride (284.53 mg, 2.242 mmol, 2.00 eq). The resulting solution was stirred for 40 min at room temperature, concentrated and used in next step directly.

### Step 3. Synthesis of 4-[[(tert-butyldiphenylsilyl)oxy]methyl]phenyl 5-[(3-[2-azatricyclo[10.4.0.0\hat{ }[4,9]]hexadeca-1(12),4(9),5,7,13,15-hexaen-2-yl]-3-oxopropyl)carbamoyl]pentanoate I-4d

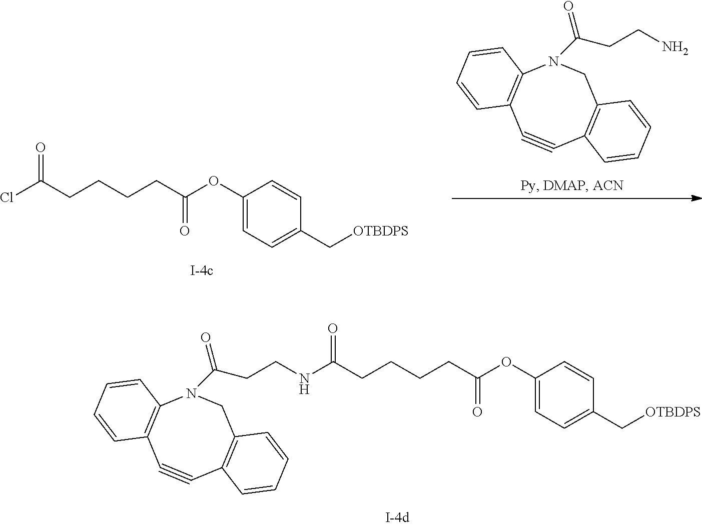

Into a 50-mL 3-necked round-bottom flask purged and maintained with an inert atmosphere of argon, were placed 3-amino-1-[2-azatricyclo[10.4.0.0\hat{ }[4,9]]hexadeca-1(12),4(9),5,7,13,15-hexaen-2-yl]propan-1-one (264.34 mg, 0.943 mmol, 1 eq), ACN (18.00 mL), pyridine (372.89 mg, 4.714 mmol, 5 eq) and DMAP (57.59 mg, 0.471 mmol, 0.5 eq) followed by 4-[[(tert-butyldiphenylsilyl)oxy]methyl]phenyl 6-chloro-6-oxohexanoate (480.00 mg, 0.943 mmol, 1.00 eq). The resulting solution was stirred for 2 hours at room temperature. The reaction was then quenched by the addition of saturated NaHCO₃ and extracted with 3×30 mL of dichloromethane. The combined organic layers were dried over anhydrous sodium sulfate, filtered and concentrated. The resulting residue was purified by Flash-Prep-HPLC eluting with ACN in water to give 400 mg (56.34%) of 4-[[(tert-butyldiphenylsilyl)oxy]methyl]phenyl 5-[(3-[2-azatricyclo[10.4.0.0\hat{ }[4,9]]hexadeca-1(12),4 (9),5,7,13,15-hexaen-2-yl]-3-oxopropyl)carbamoyl]pentanoate

### Step 4. Synthesis of 4-(hydroxymethyl)phenyl 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoate I-4

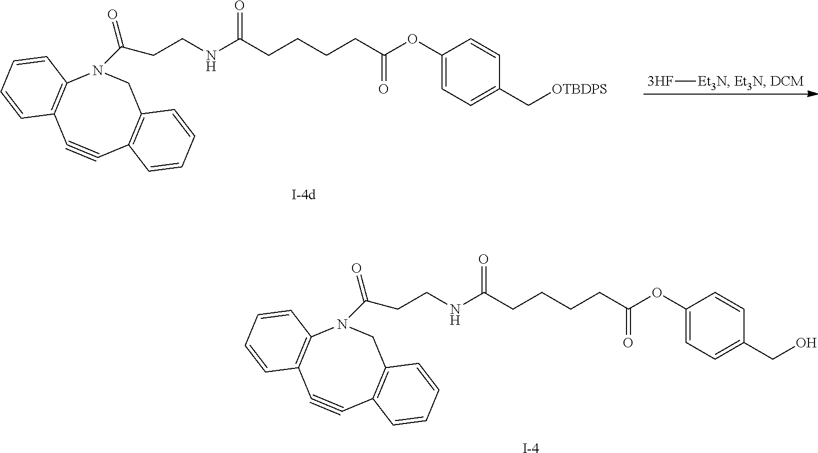

Into a 250-mL round-bottom flask purged and maintained with an inert atmosphere of nitrogen, were placed 4-[[(tert-butyldiphenylsilyl)oxy]methyl]phenyl 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoate (750.00 mg, 1.001 mmol, 1.00 eq), DCM (7.50 mL), Et₃N (506.63 mg, 5.007 mmol, 5 eq) and Et3N·3HF (968.55 mg, 6.008 mmol, 6 eq). The resulting solution was stirred overnight at room temperature. The reaction was then quenched by 200 mL of NaHCO3(aq) and extracted with 3×500 mL of ethyl acetate, the combined organic layers were dried over anhydrous sodium sulfate, filtered and concentrated. The resulting residue was purified by Flash-Prep-HPLC eluting with ACN in 10-90% water to give 152.4 mg (29.81%) of 4-(hydroxymethyl)phenyl 5-[(3-[2-azatricyclo[10.4.0.0\hat{ }[4,9]]hexadeca-1(12),4 (9),5,7,13,15-hexaen-10-yn-2-yl]-3-oxopropyl)carbamoyl]pentanoate. LC-MS (ES, m/z): 511.25[M+H]⁺; ¹H NMR (400 MHz, Methanol-d4) δ 7.74-7.64 (m, 1H), 7.52-7.25 (m, 10H), 7.14-7.00 (m, 2H), 5.16 (d, J=14.0 Hz, 1H), 4.62 (s, 2H), 3.72 (d, J=14.0 Hz, 1H), 3.27 (dd, J=13.3, 6.6 Hz, 1H), 3.21-3.11 (m, 1H), 2.58 (t, J=7.1 Hz, 2H), 2.52-2.41 (m, 1H), 2.18-2.04 (m, 3H), 1.73-1.51 (m, 4H).

### Example I-5 Synthesis of NHS-DBCO Reagent I-5

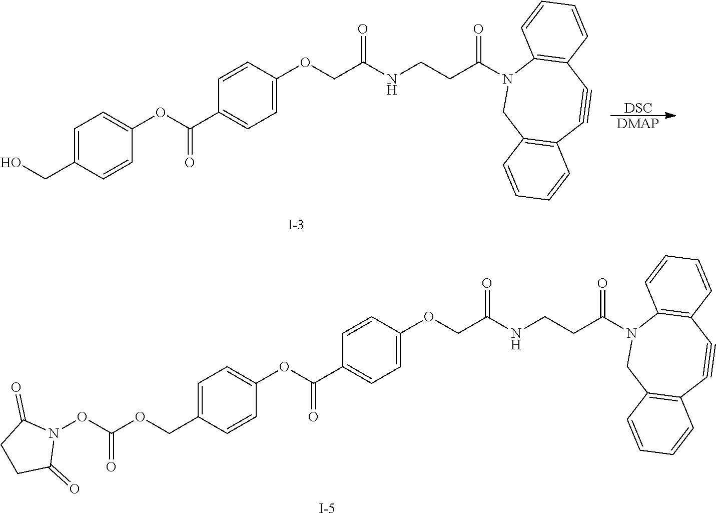

I-3 (63.3 mg, 1.0 eq.) and DMAP (6.9 mg, 0.5 eq.) were suspended in 1:1 anhydrous dichloromethane/acetonitrile (3 mL) under argon. N,N′-Disuccinimidyl carbonate (DSC) (37.7 mg, 1.3 eq.) was added to the suspension as a solid and the reaction was stirred at room temperature. After 5 h, additional N,N′-disuccinimidyl carbonate (10 mg, 0.35 eq.) was added and the reaction was stirred at 4° C. The reaction was warmed to room temperature and additional N,N′-disuccinimidyl carbonate (8.7 eq. in six portions) and DMAP (0.4 eq. in two portions) were added to the reaction to maximize conversion. The final reaction mixture was diluted with ethyl acetate (20 mL) and the resulting suspension was filtered. The filtrate was washed with 0.5 M HCl (2×10 mL) and brine (10 mL). The organic layer was separated, dried over sodium sulfate, filtered and then concentrated in vacuo to give a brown oil. The oil was dissolved in acetonitrile (4 mL), filtered (PTFE, 0.45 μm) and purified by prep-HPLC (column: Phenomenex Luna C18 150×21.2 mm, 5 μM; mobile phase: [water (0.05% TFA)-ACN]; B %: 40-80%, 25 min). Fractions containing product were concentrated in vacuo and lyophilized to give I-5 (47.2 mg, 60%). Purity 98%. MS (ES, m/z) 702.38 (M+H)⁺.

### Example I-6 Preparation of 4-(hydroxymethyl)phenyl 5-azidopentanoate I-6

### Step 1. Preparation of 5-azidopentanoyl Chloride

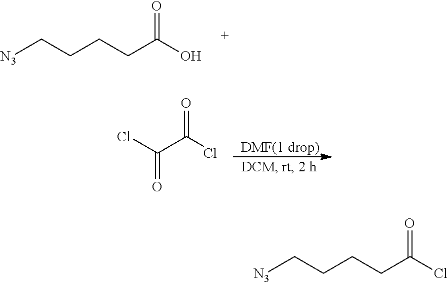 [SMILES: [N-]=[N+]=NCCCCC(=O)O ; O=C(Cl)C(=O)Cl ; [N-]=[N+]=NCCCCC(=O)Cl]

To a solution of 5-azidopentanoic acid (650 mg, 4.54 mmol, 1.0 eq) in dichloromethane (5 mL) was added a drop of dimethylformamide followed by oxalyl dichloride (1.73 g, 13.62 mmol, 3.0 eq). The mixture was stirred at room temperature for 2 h. TLC analysis of the reaction mixture showed full conversion to the desired product. Then the reaction was dried over sodium sulfate and concentrated under reduced pressure to give 5-azidopentanoyl chloride (733 mg, crude), which was used without further purification.

### Step 2. Preparation of 4-(hydroxymethyl)phenyl 5-azidopentanoate I-6

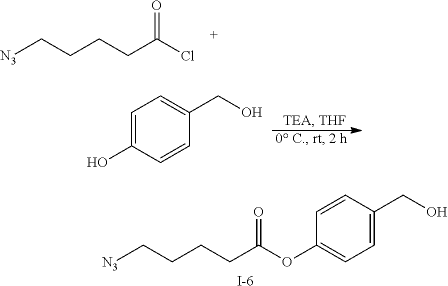

To a solution of 4-(hydroxymethyl)phenol (512.5 mg, 4.13 mmol, 1.0 eq) and triethylamine (459.5 mg, 4.54 mmol, 1.1 eq) in tetrahydrofuran (10 mL) was added 5-azidopentanoyl chloride (733 mg, 4.54 mmol, 1.1 eq) at 0° C. The resulting solution was stirred at room temperature for 2 h. The mixture was dried over sodium sulfate, filtered and concentrated under reduced pressure. The residue was purified by column chromatography on a silica gel (petroleum ether:ethyl acetate, 20:1 to 3:1) to give 4-(hydroxymethyl)phenyl 5-azidopentanoate (680 mg, 66%). TLC: petroleum ether:ethyl acetate=3:1, UV 254 nm, Rf (5-azidopentanoyl chloride)=0.1, Rf (I-6)=0.4.

### Example I-7 Preparation of 4-((((4-nitrophenoxy)carbonyl)oxy)methyl)phenyl 5-azidopentanoate

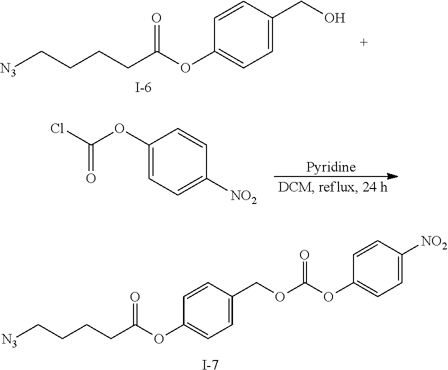

To a solution of 4-(hydroxymethyl)phenyl 5-azidopentanoate (680 mg, 2.73 mmol, 1.0 eq) in anhydrous dichloromethane (10 mL), were added 4-nitrophenyl carbonochloridate (1.27 g, 6.28 mmol, 2.3 eq) and anhydrous pyridine (540 mg, 6.83 mmol, 2.5 eq). After the solution was refluxed for 24 h, the reaction was quenched by addition of water (100 mL) and extracted with dichloromethane (3×40 mL). The combined organic layers were washed with water (3×80 mL), dried over magnesium sulfate, filtered and the filtrate was concentrated in vacuo. The resulting oil was dissolved in boiling dichloromethane (6 mL) and precipitated by 40% ethyl acetate in hexanes (20 mL). The precipitate was collected and dried to give 4-((((4-nitrophenoxy)carbonyl)oxy)methyl)phenyl 5-azidopentanoate (650 mg, crude), which was used without further purification. TLC: petroleum ether:ethyl acetate=5:1, UV 254 nm, Rf (I-6)=0.3, Rf (I-7)=0.6.

### Example I-8 Preparation of I-8

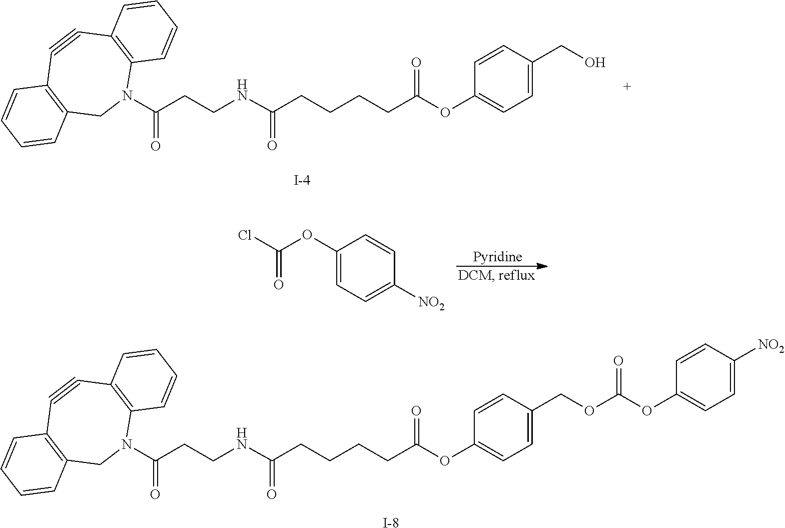

The mixture of I-4 (140 mg, 0.27 mmol, 1.0 eq), 4-nitrophenyl carbonochloridate (127.2 mg, 0.63 mmol, 2.3 eq) and pyridine (54.25 mg, 0.69 mmol, 2.5 eq) in dichloromethane (10 mL) was refluxed overnight. The mixture was dried over anhydrous sodium sulfate, filtered and concentrated under reduced pressure. The residue was purified by column chromatography on a silica gel (dichloromethane:methanol, 100:0 to 10:1) to give I-8 (83.2 mg, 44%). LC-MS (ESI, m/z): 676 [M+H]⁺. ¹HNMR (400 MHz, CDCl₃): δ 8.27-8.26 (d, J=9.2 Hz, 2H), 7.67-7.66 (d, J=7.2 Hz, 1H), 7.37 (m, 2H), 7.32-7.24 (m, 9H), 7.13-7.11 (d, J=8.4 Hz, 2H), 5.27 (s, 2H), 3.70-3.67 (m, 1H), 3.47 (s, 1H), 3.40 (m, 2H), 2.57-2.53 (m, 2H), 2.04-2.02 (m, 2H) and 1.70-1.58 (m, 4H).

### Example I-9 Preparation of I-9

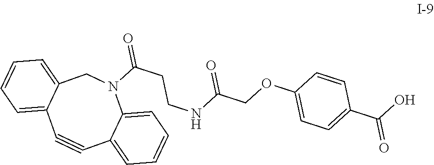 [SMILES: O=C(COc1ccc(C(=O)O)cc1)NCCC(=O)N1Cc2ccccc2C#Cc2ccccc21]

I-9 was synthesized following the procedure described in Example I-3 using I-3b and 4-hydroxybenzoic acid as starting materials. LC-MS (ESI, m/z): 455.2 [M+H]⁺. ¹HNMR (400 MHz, DMSO-d6): δ 7.93 (t, 1H), 7.85-7.87 (d, 2H), 7.27-7.66 (m, 8H), 6.90-6.92 (d, 2H), 5.03-5.07 (d, 1H), 4.42 (s, 2H), 3.62-3.66 (d, 1H), 3.22-3.27 (m, 1H), 2.99-3.05 (m, 1H), 2.45-2.51 (m, 1H), 1.84-1.89 (m, 1H).

### Example I-10 Preparation of I-10

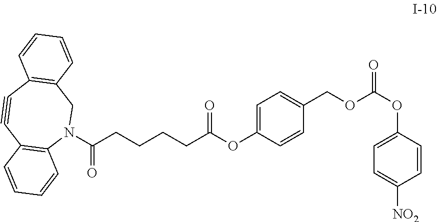 [SMILES: O=C(CCCCC(=O)N1Cc2ccccc2C#Cc2ccccc21)Oc1ccc(COC(=O)Oc2ccc([N+](=O)[O-])cc2)cc1]

### Step 1. Synthesis of I-10b

To a solution of I-10a (100 mg, 0.30 mmol) and 4-(hydroxymethyl)phenol (75 mg, mmol) in DCM (2 mL) was added DCC (93 mg, 0.45 mmol) and DMAP (55 mg, 0.45 mmol). The mixture was stirred at rt for 16 h. EtOAc (5 mL) was added. The solid was filtered and washed with EtOAc (2×2 mL). After the filtrate was concentrated, the residue was purified by prep-HPLC to provide I-10b (85 mg, 64%).

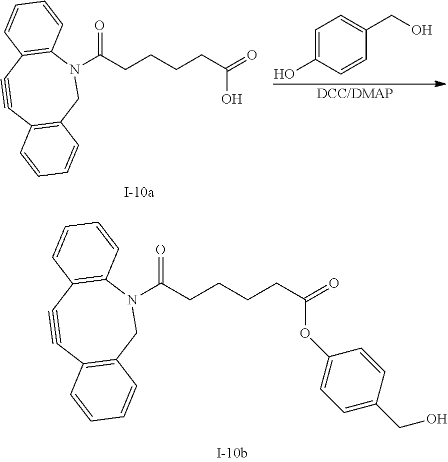

### Step 2. Synthesis of I-10

To a solution of I-10b (85 mg, 0.19 mmol) and bis(4-nitrophenyl) carbonate (20 mg, 0.064 mmol) in DCM (2 mL) was added DIEA (74 mg, 0.57 mmol). The mixture was stirred at rt for 16 h. After removal of solvent, the mixture was triturated with Et₂O (3×2 mL) to provide I-10 (90 mg), which was used without further purification. LC-MS (ESI, m/z): 605.2 [M+H]⁺.

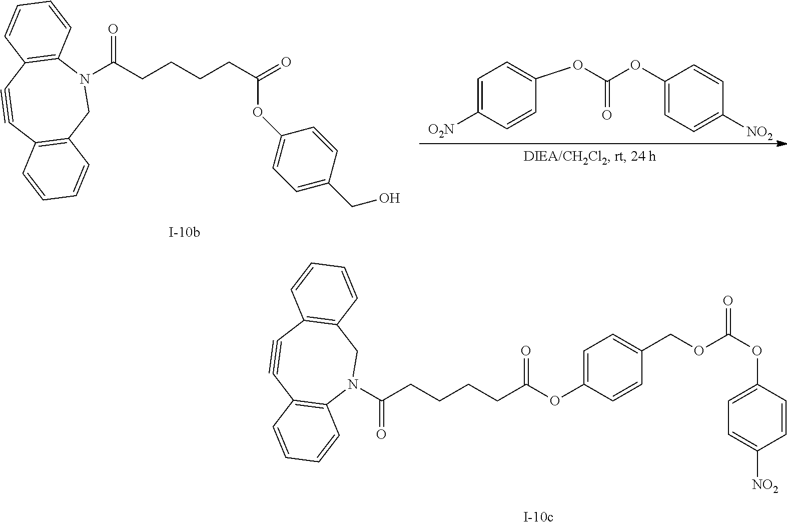 [SMILES: O=C(CCCCC(=O)N1Cc2ccccc2C#Cc2ccccc21)Oc1ccc(CO)cc1 ; O=C(Oc1ccc([N+](=O)[O-])cc1)Oc1ccc([N+](=O)[O-])cc1 ; O=C(CCCCC(=O)N1Cc2ccccc2C#Cc2ccccc21)Oc1ccc(COC(=O)Oc2ccc([N+](=O)[O-])cc2)cc1]

### Example I-11 Synthesis of 6-azido-N-(2-((4-(iodomethyl)phenyl)amino)-2-oxoethyl)hexanamide I-11

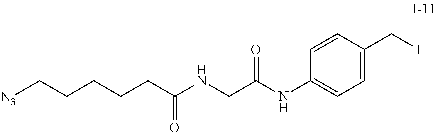 [SMILES: [N-]=[N+]=NCCCCCC(=O)NCC(=O)Nc1ccc(CI)cc1]

### Step 1. Synthesis of tert-butyl (2-((4-(hydroxymethyl)phenyl)amino)-2-oxoethyl)carbamate I-11b

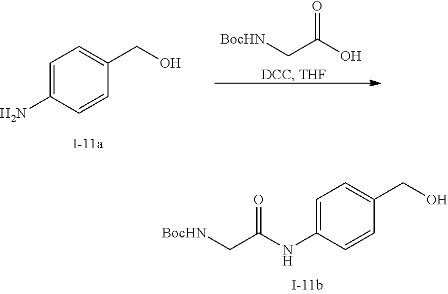 [SMILES: Nc1ccc(CO)cc1 ; CC(C)(C)OC(=O)NCC(=O)O ; CC(C)(C)OC(=O)NCC(=O)Nc1ccc(CO)cc1]

Into a 100-mL 3-necked round-bottom flask purged and maintained with an inert atmosphere of argon, were placed p-aminobenzylalcohol (2.87 g, 23.304 mmol, 1.00 eq), THF (60 mL), DCC (7.21 g, 34.956 mmol, 1.5 equiv) and [(tert-butoxycarbonyl)amino]acetic acid (4.90 g, 27.965 mmol, 1.2 eq). The resulting solution was stirred for 2 hours at room temperature. After filtration, the resulting mixture was concentrated to give a residue, which was purified by Flash-Prep-HPLC eluting with ACN in 5-95% water to provide 6 g (91.85%) of tert-butyl N-([[4-(hydroxymethyl)phenyl]carbamoyl]methyl)carbamate. LC-MS (ES, m/z): 281.20 [M+H]⁺

### Step 2. Synthesis of 2-amino-N-(4-(hydroxymethyl)phenyl)acetamide I-11c

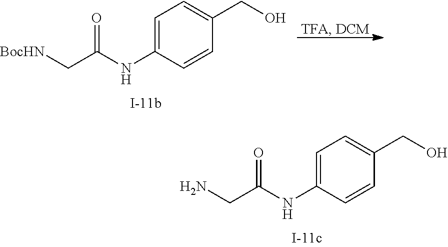 [SMILES: CC(C)(C)OC(=O)NCC(=O)Nc1ccc(CO)cc1 ; NCC(=O)Nc1ccc(CO)cc1]

To a solution of tert-butyl N-([[4-(hydroxymethyl)phenyl]carbamoyl]methyl)carbamate (200.00 mg, 0.713 mmol, 1.00 eq) in DCM (2.00 mL) at 0° C., was added TFA (1.00 mL) dropwise. The resulting solution was stirred for 1 hour at 0° C., 20 mL of ethyl ether was added and the precipitated solid was collected by filtration. The solid was then dissolved in MeOH, neutralized with SiliaBond Propylsulfonic Acid (SCX-2). The resulting mixture was concentrated to give 78 mg (60.67%) of 2-amino-N-[4-(hydroxymethyl)phenyl]acetamide. LC-MS (ES, m/z): 181.25[M+H]⁺

### Step 3. Synthesis of 6-azido-N-(2-((4-(hydroxymethyl)phenyl)amino)-2-oxoethyl)hexanamide I-11d

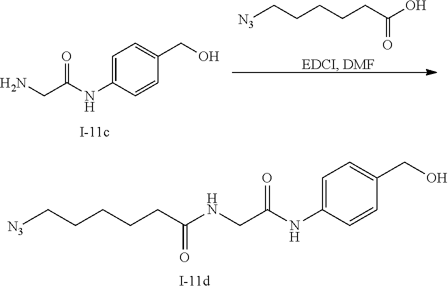 [SMILES: NCC(=O)Nc1ccc(CO)cc1 ; [N-]=[N+]=NCCCCCC(=O)O ; [N-]=[N+]=NCCCCCC(=O)NCC(=O)Nc1ccc(CO)cc1]

Into a 250-mL 3-necked round-bottom flask purged and maintained with an inert atmosphere of argon, was placed a solution of 6-azidohexanoic acid (1.74 g, 11.098 mmol, 1 equiv) in DMF (40 mL), then a solution of EDCI (2.55 g, 13.318 mmol, 1.2 eq) and 2-amino-N-[4-(hydroxymethyl) phenyl]acetamide (2.00 g, 11.098 mmol, 1.00 eq) in DMF (40.00 mL) was added dropwise. The resulting solution was stirred for 2 hours at room temperature. The reaction was then quenched by 100 mL of saturated NaHCO₃ solution and extracted with 2×100 mL of dichloromethane. The combined organic layers were washed with 2 x100 ml of brine, dried and concentrated. The crude product was purified by Flash-Prep-HPLC eluting with ACN in 5-95% water to give 3.4 g (95.93%) of 6-azido-N-([[4-(hydroxymethyl)phenyl]carbamoyl]methyl)hexanamide. LC-MS (ES, m/z): 320.00[M+H]⁺

### Step 4. Synthesis of 6-azido-N-(2-((4-(chloromethyl)phenyl)amino)-2-oxoethyl)hexanamide I-11e

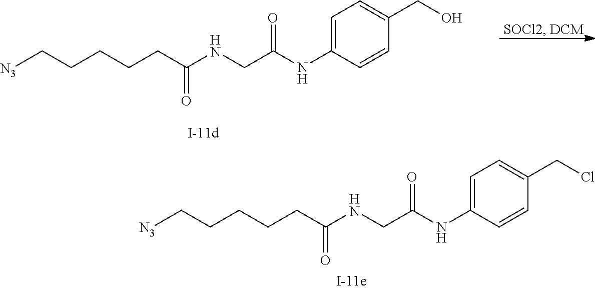 [SMILES: [N-]=[N+]=NCCCCCC(=O)NCC(=O)Nc1ccc(CO)cc1 ; [N-]=[N+]=NCCCCCC(=O)NCC(=O)Nc1ccc(CCl)cc1]

Under argon atmosphere, to a solution of 6-azido-N-([[4-(hydroxymethyl)phenyl]carbamoyl]methyl)hexanamide (500.00 mg, 1.566 mmol, 1.00 eq) in DCM (5.00 mL) at 0° C., was added SOCl₂ (372.52 mg, 3.131 mmol, 2.00 eq) dropwise. The resulting solution was stirred for 40 min at 0° C. and then quenched by 10 mL of saturated NaHCO₃ solution. The resulting mixture was extracted with 2×10 mL of dichloromethane, the combined organic layers were dried over anhydrous sodium sulfate, filtered and concentrated to give the 500 mg of crude product, which was directly used it in the next step. LC-MS (ES, m/z): 338.15[M+H]⁺

### Step 5. Synthesis of 6-azido-N-(2-((4-(iodomethyl)phenyl)amino)-2-oxoethyl)hexanamide I-11

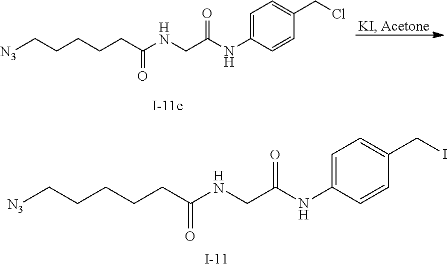

Into a 25-mL round-bottom flask, were placed 6-azido-N-([[4-(chloromethyl)phenyl]carbamoyl]methyl)hexanamide (200.00 mg, 0.592 mmol, 1.00 eq), acetone (2.00 mL) and KI (393.13 mg, 2.368 mmol, 4.00 eq). The resulting solution was stirred for 4 hours at room temperature. After filtration, the resulting mixture was concentrated to give a residue, which was purified by chromatography eluting with ethyl acetate/petroleum ether (4:1) to provide 100 mg (39.35%) of 6-azido-N-([[4-(iodomethyl)phenyl]carbamoyl]methyl)hexanamide. LC-MS (ES, m/z): 430.10 [M+H]⁺

### Example I-12 Synthesis of 1-(2-(2-(2-azidoethoxy)ethoxy)ethoxy)-4-(iodomethyl)benzene I-12

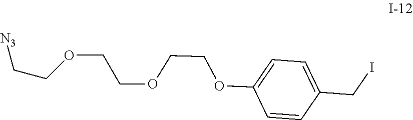 [SMILES: [N-]=[N+]=NCCOCCOCCOc1ccc(CI)cc1]

### Step 1. Synthesis of (4-(2-(2-(2-azidoethoxy)ethoxy)ethoxy)phenyl)methanol

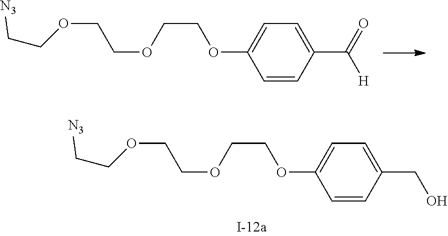 [SMILES: [N-]=[N+]=NCCOCCOCCOc1ccc(C=O)cc1 ; [N-]=[N+]=NCCOCCOCCOc1ccc(CO)cc1]

To a solution of 4-(2-(2-(2-azidoethoxy)ethoxy)ethoxy)benzaldehyde (1.0 g, 3.58 mmol) in 20 mL MeOH at 0° C., is added NaBH4 (163 mg, 4.30 mmol) slowly, the mixture is stirred for 1h at room temperature and then concentrated in vacuo. The resulting residue is diluted with 20 mL H₂O and extracted into EtOAc (3×20 mL). The combined organic layers are washed with brine (2×5 mL), dried with Na₂SO₄, filtered and concentrated to yield I-12a.

### Step 2. 1-(2-(2-(2-azidoethoxy)ethoxy)ethoxy)-4-(iodomethyl)benzene I-12 is Prepared Following the Procedure as Described in Step 4 and 5 of Example I-11

### Example I-13 Synthesis of I-13

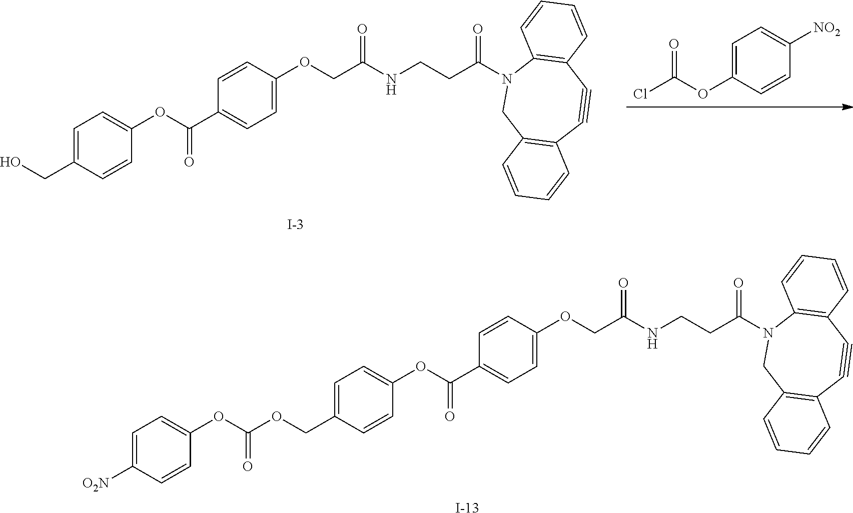

I-13 was synthesized by the reaction of I-3 with 4-nitrophenyl carbonochloridate according to the procedure described in Example I-8. LC-MS (ESI, m/z): 726.20 [M+H]⁺.

### Example I-14 Synthesis of I-14

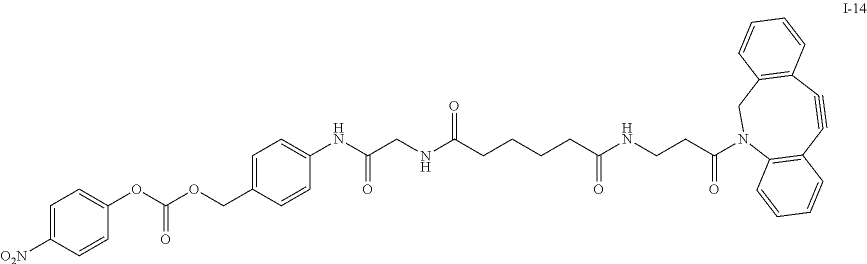 [SMILES: O=C(CCCCC(=O)NCC(=O)Nc1ccc(COC(=O)Oc2ccc([N+](=O)[O-])cc2)cc1)NCCC(=O)N1Cc2ccccc2C#Cc2ccccc21]

### Step 1. Synthesis of 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoic Acid I-14a

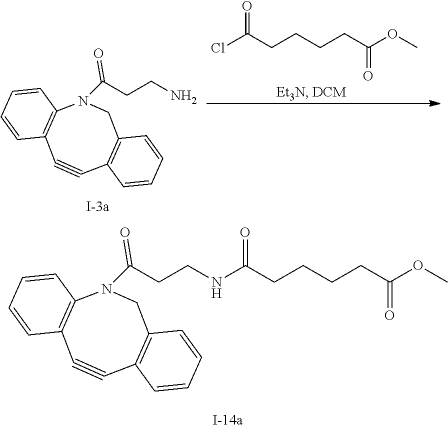 [SMILES: NCCC(=O)N1Cc2ccccc2C#Cc2ccccc21 ; COC(=O)CCCCC(=O)Cl ; COC(=O)CCCCC(=O)NCCC(=O)N1Cc2ccccc2C#Cc2ccccc21]

To a solution of 3-amino-1-[2-azatricyclo[10.4.0.0\hat{ }[4,9]]hexadeca-1(12),4(9),5,7,13,15-hexaen-10-yn-2-yl]propan-1-one (2000.00 mg, 7.237 mmol, 1.00 eq) and Et₃N (2197.08 mg, 21.712 mmol, 3.00 eq) in DCM (40.00 mL), was added methyl 6-chloro-6-oxohexanoate (1939.03 mg, 10.856 mmol, 1.50 eq) dropwise at 0° C., the solution was stirred for 30 min. at 0° C. and additional 1 hour at room temperature. The reaction was quenched by 100 mL of saturated NaHCO₃ and extracted with 3×100 mL of dichloromethane, the combined organic layers were dried over anhydrous sodium sulfate. After filtration, the filtrate was concentrated under vacuum to provide the crude product, which was purified by chromatography eluting with petroleum/ethyl acetate to give 2000 mg (68.32%) of 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoic acid. LC-MS [M+H]+ 419.25.

### Step 2. Synthesis of 5-[(3-[2-azatricyclo[10.4.0.0\hat{ }[4,9]]hexadeca-1(12),4 (9),5,7,13,15-hexaen-10-yn-2-yl]-3-oxopropyl)carbamoyl]pentanoic Acid I-14b

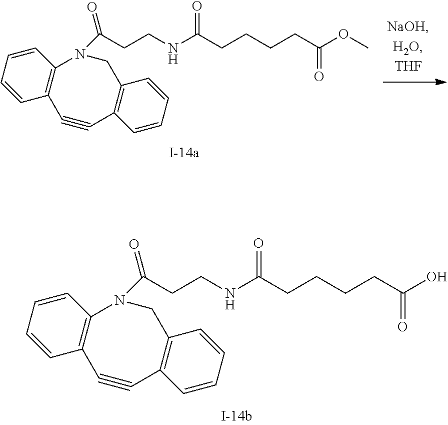 [SMILES: COC(=O)CCCCC(=O)NCCC(=O)N1Cc2ccccc2C#Cc2ccccc21 ; O=C(O)CCCCC(=O)NCCC(=O)N1Cc2ccccc2C#Cc2ccccc21]

To a solution of methyl 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoate (2.00 g, 4.779 mmol, 1.00 eq) in THF (50.00 mL), was added NaOH (382.30 mg, 9.558 mmol, 2.00 eq) in H₂O (10.00 mL) at 0° C., then stirred for 5 min. The resulting solution was stirred overnight at room temperature and adjust to pH=2 with Dowex(H+) at room temperature. The resulting mixture was concentrated under vacuum to give 1800 mg (93.12%) of 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoic acid. LC-MS [M+H]⁺ 405.20

### Step 3. Synthesis of ([[4-(hydroxymethyl)phenyl]carbamoyl]methyl)amino 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoate I-14c

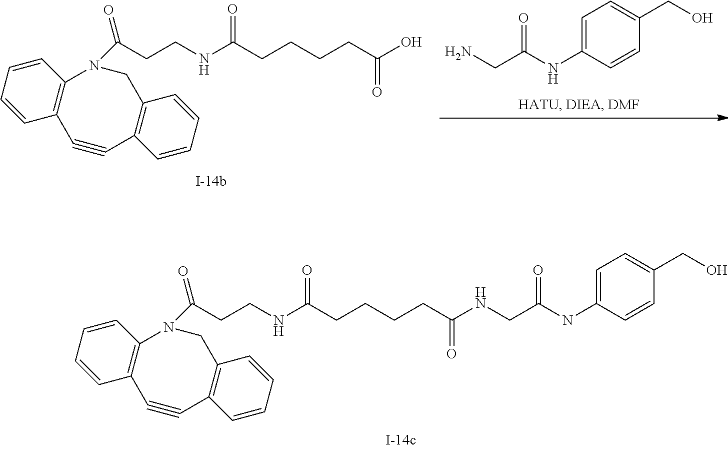

To a solution of 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoic acid (1000.00 mg, 2.472 mmol, 1.00 eq) in DMF (100.00 mL, 1368.103 mmol, 522.64 eq), were added HATU (1128.09 mg, 2.967 mmol, 1.20 eq) and DIEA (639.08 mg, 4.945 mmol, 2 eq), the mixture was stirred at rt for 10 min followed by addition of 2-amino-N-[4-(hydroxymethyl)phenyl]acetamide (534.65 mg, 2.967 mmol, 1.20 eq). The resulting solution was stirred overnight at room temperature, quenched by 150 mL of water and extracted with 3×100 mL of dichloromethane. The combined organic layers were dried over anhydrous sodium sulfate, filtered and concentrated under vacuum, the resulting residue was purified by Flash-Prep-HPLC eluting with ACN in 70-95% water to give 1200 mg (83.30%) of ([[4-(hydroxymethyl)phenyl]carbamoyl]methyl)amino 5-[(3-[2-azatricyclo[10.4.0.0\hat{ }[4,9]]hexadeca-1(12),4(9),5,7,13,15-hexaen-10-yn-2-yl]-3-oxopropyl)carbamoyl]pentanoate. LC-MS [M+H]⁺ 567.35. ¹H NMR (300 MHz, DMSO-d6) δ 9.90 (s, 1H), 8.08 (t, J=5.9 Hz, 1H), 7.73-7.14 (m, 11H), 5.13-4.99 (m, 2H), 4.42 (d, J=Hz, 2H), 3.84 (d, J=5.8 Hz, 2H), 3.62 (d, J=13.9 Hz, 2H), 3.01 (dt, J=45.9, 6.7 Hz, 2H), 2.09 (d, J=9.7 Hz, 2H), 1.92 (s, 2H), 1.39 (m, 4H).

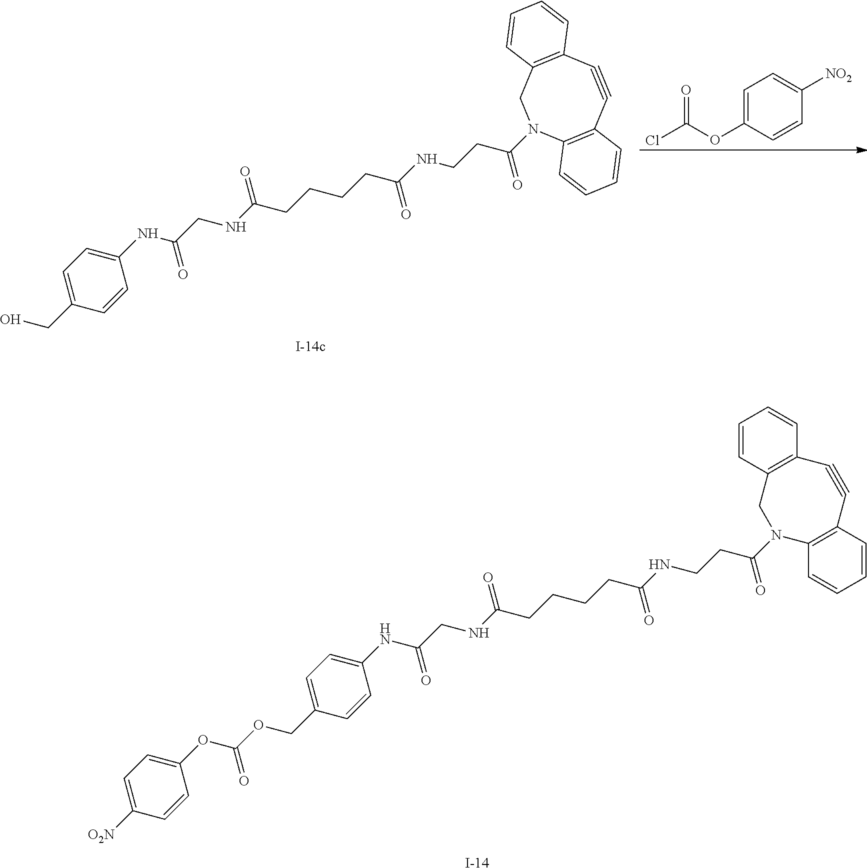

I-14 was synthesized by the reaction of I-14c with 4-nitrophenyl carbonochloridate according to the procedure described in Example I-8. LC-MS (ESI, m/z): 732.4 [M+H]⁺.

### Example I-15 Synthesis of 6-azido-N—((S)-1-(((S)-1-((4-(iodomethyl)phenyl)amino)-1-oxopropan-2-yl)amino)-3-methyl-1-oxobutan-2-yl)hexanamide

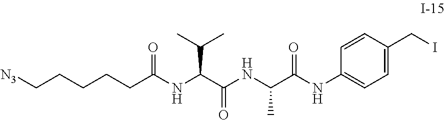 [SMILES: CC(C)[C@H](NC(=O)CCCCCN=[N+]=[N-])C(=O)N[C@@H](C)C(=O)Nc1ccc(CI)cc1]

### Step 1. Synthesis of (9H-fluoren-9-yl)methyl (S)-(1-((4-(hydroxymethyl)phenyl)amino)-1-oxopropan-2-yl)carbamate I-15b

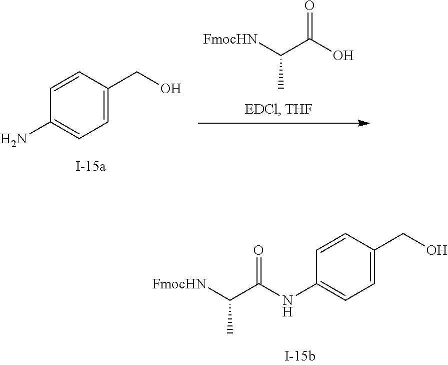

Into a 500-mL 3-necked round-bottom flask purged and maintained with an inert atmosphere of argon, was placed a solution of (2S)-2-[[(9H-fluoren-9-ylmethoxy)carbonyl]amino]propanoic acid (35.49 g, 114.003 mmol, 1.20 eq) and EDCI (27.32 g, 142.513 mmol, 1.50 eq) in THF (150 mL), p-aminobenzylalcohol (11.7 g, 95.002 mmol, 1.00 equiv) in THF (150 mL) was added dropwise. The resulting solution was stirred for 2 hours at room temperature, quenched by the addition of 500 mL of saturated NaHCO₃ solution and extracted with 2×500 mL of ethyl acetate. The combined organic layers were washed with 2×500 ml of brine, dried over anhydrous sodium sulfate, filtered and concentrated to give 29.7 g (75.06%) of 9H-fluoren-9-ylmethyl N-[(1S)-1-[[4-(hydroxymethyl)phenyl]carbamoyl]ethyl]carbamate as a solid. LC-MS (ES, m/z): 417.05 [M+H]⁺.

### Step 2. Synthesis of (S)-2-amino-N-(4-(hydroxymethyl)phenyl)propenamide I-15c

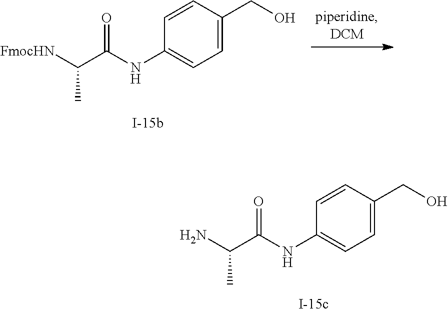

The mixture of 9H-fluoren-9-ylmethyl N-[(1S)-1-[[4-(hydroxymethyl)phenyl]carbamoyl]ethyl]carbamate (5.00 g, 2.041 mmol, 1.00 eq) and piperidine (16.00 mL) in DCM (50.00 mL) was stirred for 30 min at room temperature. Solid was precipitated by addition of diethyl ether and filtered. The filtrate was concentrated to give 1.8 g of (2S)-2-amino-N-[4-(hydroxymethyl)phenyl]propanamide as a solid. LC-MS (ES, m/z): 195.20 [M+H]⁺

### Step 3. Synthesis of (9H-fluoren-9-yl)methyl ((S)-1-(((S)-1-((4-(hydroxymethyl)phenyl)amino)-1-oxopropan-2-yl)amino)-3-methyl-1-oxobutan-2-yl)carbamate I-15d

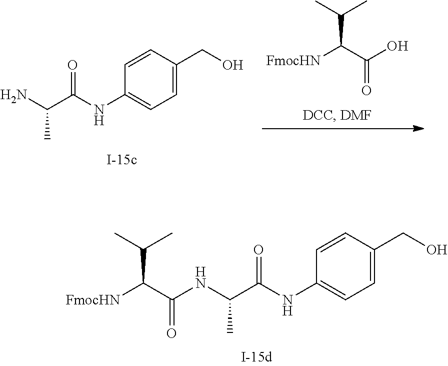

Into a 250-mL round-bottom flask purged and maintained with an inert atmosphere of argon, was placed a solution of (2S)-2-amino-N-[4-(hydroxymethyl)phenyl]propanamide (5.00 g, 25.742 mmol, 1.00 eq), DCC (7.97 g, 38.613 mmol, 1.5 eq) and (2S)-2-[[(9H-fluoren-9-ylmethoxy)carbonyl]amino]-3-methylbutanoic acid (10.48 g, 30.891 mmol, 1.2 equiv) in DMF (50.00 mL). The resulting solution was stirred for 2 hours at room temperature. After filtration, the filtrate was then quenched by the addition of 100 mL of water/ice and extracted with 2×100 mL of ethyl acetate, the organic layers were dried over anhydrous sodium sulfate and concentrated to obtain a residue, which was purified by Flash-Prep-HPLC eluting with ACN in 5-95% water to give 9.9 g (74.59%) of 9H-fluoren-9-ylmethyl N-[(1S)-1-[[(1S)-1-[[4-(hydroxymethyl)phenyl]carbamoyl]ethyl]carbamoyl]-2-methylpropyl]carbamate as a solid. LC-MS (ES, m/z): 516.30 [M+H]⁺.

### Step 4. Synthesis of (S)-2-amino-N—((S)-1-((4-(hydroxymethyl)phenyl)amino)-1-oxopropan-2-yl)-3-methylbutanamide I-15e

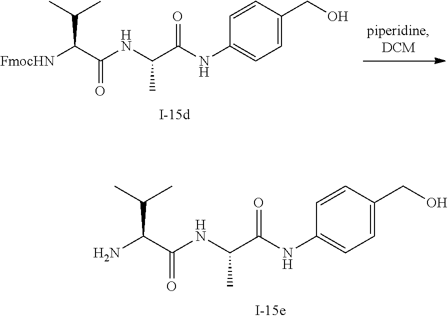

The solution of 9H-fluoren-9-ylmethyl N-[(1S)-1-[[(1S)-1-[[4-(hydroxymethyl) phenyl]carbamoyl]ethyl]carbamoyl]-2-methylpropyl]carbamate (2.60 g, 5.043 mmol, 1.00 eq) and piperidine (9.00 mL) in DCM (26.00 mL) was stirred for 30 min at room temperature. The resulting mixture was concentrated. The crude product was purified by Flash-Prep-HPLC eluting with ACN in 5%-95% water to give 1.19 g (80.44%) of (2S)-2-amino-N-[(1S)-1-[[4-(hydroxymethyl)phenyl]carbamoyl]ethyl]-3-methylbutanamide as a solid. LC-MS (ES, m/z): 294.20[M+H]⁺

### Step 5. Synthesis of 6-azido-N—((S)-1-(((S)-1-((4-(hydroxymethyl)phenyl)amino)-1-oxopropan-2-yl)amino)-3-methyl-1-oxobutan-2-yl)hexanamide I-15f

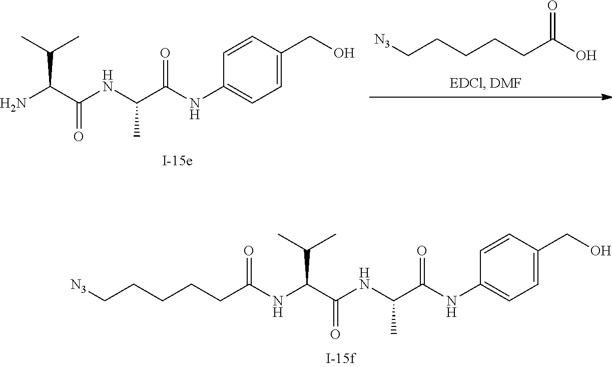

Into a 50-mL 3-necked round-bottom flask purged and maintained with an inert atmosphere of argon, was placed a solution of (2S)-2-amino-N-[(1S)-1-[[4-(hydroxymethyl)phenyl]carbamoyl]ethyl]-3-methylbutanamide (1.50 g, 5.113 mmol, 1.00 eq), EDCI (1.47 g, 7.670 mmol, 1.50 equiv) and 6-azidohexanoic acid (0.96 g, 6.108 mmol, 1.19 eq) in DMF (15.00 mL). The resulting solution was stirred for 2 hours at room temperature, quenched by the addition of 100 mL of water/ice and extracted with 2×100 mL of ethyl acetate. The combined organic layers were dried over anhydrous sodium sulfate and concentrated. The crude product was purified by Flash-Prep-HPLC eluting with ACN in 5%-95% water to give 1.88 g (85.0%) of 6-azido-N-[(1 S)-1-[[(1 S)-1-[[4-(hydroxymethyl)phenyl]carbamoyl]ethyl]carbamoyl]-2-methylpropyl]hexanamide as a solid. LC-MS (ES, m/z): 433.10 [M+H]; ¹H-NMR (300 MHz, DMSO-d6) δ 9.85 (s, 1H), 8.14 (d, J=7.1 Hz, 1H), 7.85 (d, J=8.5 Hz, 1H), 7.58-7.49 (m, 2H), 7.23 (d, J=8.4 Hz, 2H), 5.08 (s, 1H), 4.45-4.31 (m, 3H), 4.17 (dd, J=8.5, 6.8 Hz, 1H), 2.16 (m, J=7.1 Hz, 2H), 1.96 (dt, J=13.7, 6.8 Hz, 1H), 1.60-1.43 (m, 5H), 1.30 (d, J=7.1 Hz, 5H), 0.85 (dd, J=10.3, 6.7 Hz, 6H).

### Step 6. Synthesis of 6-azido-N—((S)-1-(((S)-1-((4-(iodomethyl)phenyl)amino)-1-oxopropan-2-yl)amino)-3-methyl-1-oxobutan-2-yl)hexanamide I-15

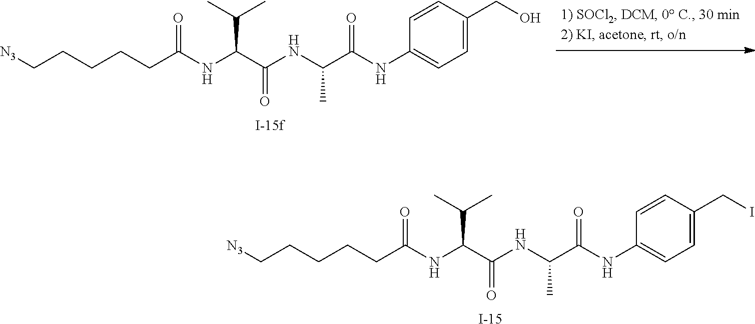

To a solution of 6-azido-N-[(1S)-1-[[(1S)-1-[[4-(hydroxymethyl)phenyl]carbamoyl]ethyl]carbamoyl]-2-methylpropyl]hexanamide (500.00 mg, 1.15 mmol, 1.00 eq) in DCM (5.00 mL) under argon atmosphere at 0° C., was added SOCl₂ (270.52 mg, 2.31 mmol, 2.00 eq) dropwise. The resulting solution was stirred for 40 min at 0° C., quenched by 10 mL of saturated NaHCO₃ solution and extracted with 2×10 mL of dichloromethane, the combined organic layers were dried over anhydrous sodium sulfate and concentrated. 500 mg (crude) chloride was obtained, which was used in the next step.

The mixture of 6-azido-N—((S)-1-((S)-1-(4-(chloromethyl)phenylamino)-1-oxopropan-2-ylamino)-3-methyl-1-oxobutan-2-yl)hexanamide (500.00 mg, 0.92 mmol, 1.00 eq) and KI (612.13 mg, 3.69 mmol, 4.00 eq) in acetone (2.00 mL) was stirred for 4 hours at room temperature. After filtration, the filtrate was concentrated to give a residue, which was purified by chromatography eluting with ethyl acetate/petroleum ether (4:1) to provide 100 mg (39.35%) of 6-azido-N—((S)-1-((S)-1-(4-(iodomethyl)phenylamino)-1-oxopropan-2-ylamino)-3-methyl-1-oxobutan-2-yl)hexanamide as a solid. ¹H NMR (400 MHz, Chloroform-d) δ 8.78 (s, 1H), 7.52-7.43 (m, 2H), 7.34-7.29 (m, 2H), 6.50 (d, J=8.5 Hz, 1H), 4.78-4.70 (m, 1H), 4.51-4.39 (m, 3H), 3.24 (t, J=6.8 Hz, 2H), 2.30 (td, J=7.4, 3.9 Hz, 2H), 2.06 (dt, J=13.8, 6.8 Hz, 1H), 1.68 (m, 2H), 1.60 (s, 4H), 1.46 (d, J=7.0 Hz, 3H), 1.43-1.36 (m, 2H), 0.97-0.91 (m, 6H).

### Example I-16 Synthesis of 1-((2-(2-(2-azidoethoxy)ethoxy)ethoxy)methyl)-4-(chloromethyl)benzene

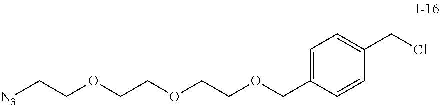 [SMILES: [N-]=[N+]=NCCOCCOCCOCc1ccc(CCl)cc1]

### Step 1. Synthesis of (4-(((tert-butyldimethylsilyl)oxy)methyl)phenyl)methanol

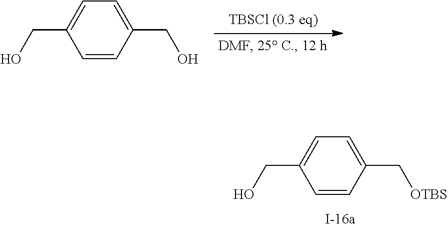 [SMILES: OCc1ccc(CO)cc1 ; CC(C)(C)[Si](C)(C)OCc1ccc(CO)cc1]

1,4-Phenylenedimethanol (10.0 g, 72.3 mmol, 1.00 eq) was dissolved in DMF (500 mL) at 25° C. under N₂. Imidazole (4.93 g, 72.3 mmol, 1.00 eq) was added to above solution followed by TBSCl (3.27 g, 21.7 mmol, 2.66 mL, 0.300 eq). The mixture was stirred at rt for 12 hrs. The solution was diluted with H₂O (500 mL), extracted with EtOAc (500 mL×2), the organic layer was washed with brine (500 mL), dried with Na₂SO₄, filtered and concentrated with water pump under 35° C. to give an oil, which was purified by column chromatography (SiO₂, Petroleum ether/Ethyl acetate=100/1 to 10/1) to give compound I-16a (3.40 g, 13.4 mmol, 18.6% yield) as an oil. ¹H NMR (400 MHz, CDCl₃) 7.34 (s, 4H), 4.74 (s, 2H), 4.67 (s, 2H), 1.64 (s, 1H), 0.94 (s, 9H), 0.10 (s, 6H).

### Step 2. Synthesis of tert-butyl((4-(iodomethyl)benzyl)oxy)dimethylsilane

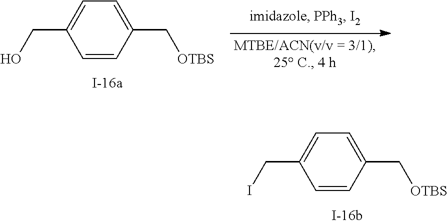 [SMILES: CC(C)(C)[Si](C)(C)OCc1ccc(CO)cc1 ; CC(C)(C)[Si](C)(C)OCc1ccc(CI)cc1]

Imidazole (1.02 g, 15.0 mmol, 2.00 eq) and PPh₃ (2.96 g, 11.3 mmol, 1.50 eq) was dissolved in MTBE/ACN (v/v=3/1, 53 mL) at 20-23° C. under N₂. I₂ (2.87 g, 11.3 mmol, 2.27 mL, 1.50 eq) was added to the above solution to give a yellow suspension at 20-23° C. Compound I-16a (1.90 g, 7.53 mmol, 1.00 eq) in MTBE/ACN (v/v=3/1, 4 mL) was added to above suspension. The mixture was stirred at 20-23° C. for 4 hrs. The organic phase was concentrated with water pump under 35° C. to give a yellow residue, to which a mixture of hexane (150 mL) and THF (50 ml) was added followed by NaHSO3 aqueous solution. The organic phase was dried with anhydrous Na₂SO₄, filtered, concentrated with water pump under 35° C. to give a residue, which was purified by column chromatography (SiO₂, Petroleum ether/ethyl acetate=100/1-10/1) to give compound I-16b (1.50 g, 4.14 mmol, 55.0% yield) as an oil. ¹H NMR (400 MHz, CDCl₃) 7.27-7.25 (d, 2H), 7.17-7.15 (d, 2H), 4.61 (s, 2H), 4.38 (s, 2H), 0.85 (s, 9H), 0.01 (s, 6H).

### Step 3. Synthesis of ((4-((2-(2-(2-azidoethoxy)ethoxy)ethoxy)methyl)benzyl)oxy)(tert-butyl)dimethylsilane

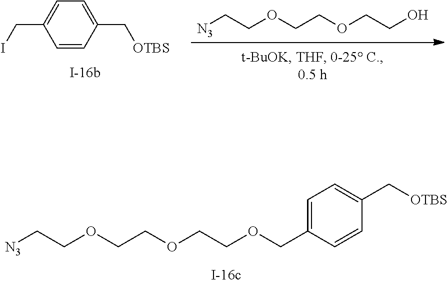 [SMILES: CC(C)(C)[Si](C)(C)OCc1ccc(CI)cc1 ; [N-]=[N+]=NCCOCCOCCO ; CC(C)(C)[Si](C)(C)OCc1ccc(COCCOCCOCCN=[N+]=[N-])cc1]

To a solution of 2-(2-(2-azidoethoxy)ethoxy)ethan-1-ol (1.22 g, 6.98 mmol, 1.10 eq) in THF (13.8 mL), was added t-BuOK (1 M, 9.52 mL, 1.50 eq) at 0° C. under N₂. The solution was stirred for 10 min at 0° C. Compound I-16b (2.30 g, 6.35 mmol, 1.00 eq) in THF (6.9 mL) was added to the above solution at 0° C. The mixture was stirred at 27° C. for 4 hrs. The solution was concentrated with water pump under 40° C. to give a solid, which was purified by column chromatography (SiO₂, Petroleum ether/Ethyl acetate=50/1 to 10/1) to give compound I-16c (1.78 g, 4.35 mmol, 68.4% yield) as an oil. ¹H NMR (400 MHz, CDCl₃) 7.30 (s, 4H), 4.74 (s, 2H), 4.56 (s, 2H), 3.69-3.67 (m, 8H), 3.64-3.62 (m, 2H), 3.61-3.38 (t, J=5.2 Hz, 2H), 0.94 (s, 9H), 0.10 (s, 6H).

### Step 4. Synthesis of (4-((2(2-(2-azidoethoxy)ethoxy)ethoxy)methyl)phenyl)methanol

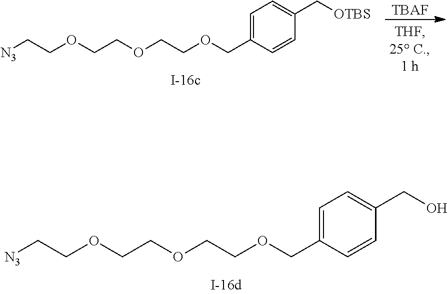

To a solution of compound I-16c (1.78 g, 4.35 mmol, 1.00 eq) in THF (10 mL), was added TBAF (1 M, 2.17 mL, 0.500 eq) at 25° C. under N₂. The solution was stirred for 1 hour at 25° C. After concentration, the resulting residue was purified by column chromatography (SiO₂, Petroleum ether/Ethyl acetate=5/1 to 1/3) to give compound I-16d (1.07 g, 3.62 mmol, 83.3% yield) as an oil. ¹H NMR (400 MHz, CDCl₃) 7.34 (s, 4H), 4.68 (s, 2H), 4.56 (s, 2H), 3.70-3.67 (m, 8H), 3.64-3.63 (m, 2H), 3.39-3.36 (t, J=5.2 Hz, 2H).

### Step 5. Synthesis of 1-((2-(2-(2-azidoethoxy)ethoxy)ethoxy)methyl)-4-(chloromethyl)benzene

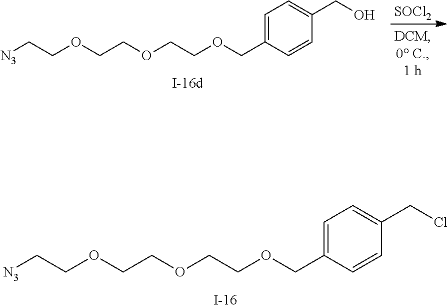

Compound I-16d (62.0 mg, 209 umol, 1.00 eq) was dissolved in DCM (1 mL), and the solution was cooled to 0° C. Then S0C12 (49.9 mg, 419 umol, 30.4 uL, 2.00 eq) was added dropwise at 0° C. The mixture was stirred at 0° C. for 1 hr. The solution was quenched with aq. NaHCO₃ (20 mL), extracted with DCM (20 mL×2). The combined organic layers were dried with Na₂SO₄, filtered and concentrated to give a residue (65.8 mg, crude), which was used into the next step without further purification.

### Example I-17 Synthesis of I-17

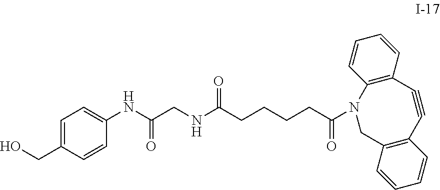 [SMILES: O=C(CCCCC(=O)N1Cc2ccccc2C#Cc2ccccc21)NCC(=O)Nc1ccc(CO)cc1]

### Step 1. Synthesis of tert-butyl (2-((4-(hydroxymethyl)phenyl)amino)-2-oxoethyl)carbamate

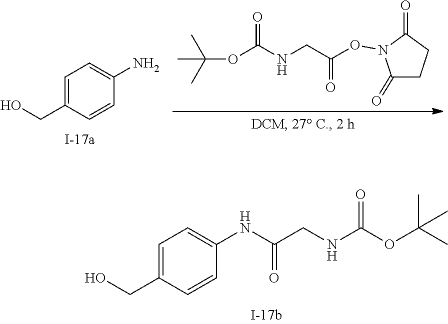 [SMILES: Nc1ccc(CO)cc1 ; CC(C)(C)OC(=O)NCC(=O)ON1C(=O)CCC1=O ; CC(C)(C)OC(=O)NCC(=O)Nc1ccc(CO)cc1]

To a solution of (4-aminophenyl)methanol (5.00 g, 40.6 mmol, 1.00 eq) in DCM (30 ml) was added 2,5-dioxopyrrolidin-1-yl (tert-butoxycarbonyl)glycinate (44.2 g, 162 mmol, 4.00 eq) at 27° C. under N₂. The mixture was stirred at 27° C. for 5.5 hrs. The mixture was filtered and the organic phase was concentrated with water pump under 35° C. to give an oil, which was purified by column chromatography (SiO₂, DCM:MeOH=1:0 to 10:1) to give I-17b (8.00 g, crude) as a solid.

### Step 2. Synthesis of 2-amino-N-(4-(hydroxymethyl)phenyl)acetamide

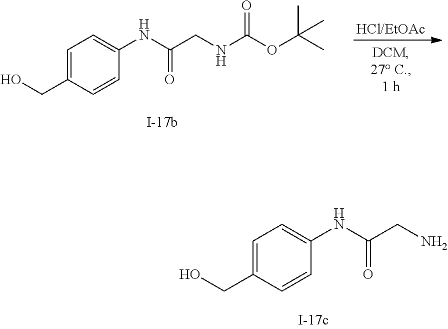 [SMILES: CC(C)(C)OC(=O)NCC(=O)Nc1ccc(CO)cc1 ; NCC(=O)Nc1ccc(CO)cc1]

4M HCl in EtOAc (22.5 mL, 6.73 eq) was added to I-17b (3.75 g, 13.3 mmol, 1.00 eq) at 25° C. under N₂. The mixture was stirred at 25° C. for 0.5 hr. The mixture was filtered and the organic phase was concentrated with water pump under 35° C. to give a residue, which was purified by prep-HPLC (TFA condition, column: Xtimate C18 250 mm*80 mm*10 um; mobile phase: [water (0.1% TFA)-ACN]; B %: 0%-30%, 10 min) to give I-17c (2.00 g, 11.1 mmol, 40.0% yield) as a solid. ¹HNMR (400 MHz, DMSO-d₆) 10.59 (s, 1H), 8.20 (s, 3H), 7.56-7.54 (d, J=8.4 Hz, 2H), 7.29-7.27 (d, J=8.4 Hz, 2H), 4.44 (s, 2H), 3.77-3.74 (s, 2H).

### Step 3. Synthesis of I-17

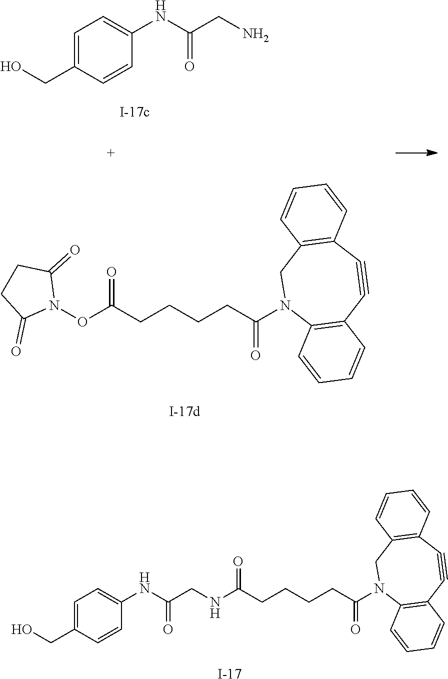

To a solution of I-17c (121 mg, 671 umol, 1.00 eq) in DMF (1 mL) was added I-17d (289 mg, 671 umol, 1.00 eq) at 25-27° C. under N₂. The solution was stirred at 25-27° C. for 1 h. The reaction mixture was diluted with DCM (5 mL) and washed with water and brine, dried, filtered and concentrated under reduced pressure to give a residue, which was purified by prep-HPLC (neutral condition, YMC Triart C18 150*25 mm*Sum; mobile phase: [water (10 mM NH₄HCO3)-ACN]; B %: 25%-55%, 10 min) to give I-17 (57.5 mg, 116 umol, 20.8% yield) as a solid. LC/MS (ES, m/z): 496.2 (M+H)⁺. ¹HNMR (400 MHz, DMSO-d₆) 9.86 (s, 1H), 8.15-7.89 (m, 1H), 7.63 (d, J=7.6 Hz, 1H), 7.60-7.56 (m, 1H), 7.54-7.43 (m, 5H), 7.42-7.27 (m, 3H), 7.22 (d, J=8.4 Hz, 2H), 5.21 (s, 1H), 5.23-4.93 (m, 1H), 4.42 (d, J=6.0 Hz, 2H), 3.78 (d, J=6.0 Hz, 2H), 3.61 (d, J=14.0 Hz, 1H), 3.30 (br s, 1H), 2.23-2.12 (m, 1H), 1.94 (br t, J=7.2 Hz, 2H), 1.76 (m, 1H), 1.39-1.28 (m, 1H), 1.27-1.13 (m, 3H).

### Example 1 Synthesis of ((2R,3R,3aR,7aR,9R,10R,10aR,12S,14aR)-2-(2-amino-6-oxo-1,6-dihydro-9H-purin-9-yl)-9-(6-amino-9H-purin-9-yl)-3,10-difluoro-12-mercapto-5,12-dioxidooctahydro-2H,7H-difuro[3,2-d:3′,2′-j][1,3,7,9]tetraoxa[2,8]diphosphacyclododecin-5-yl)trihydroborate A1

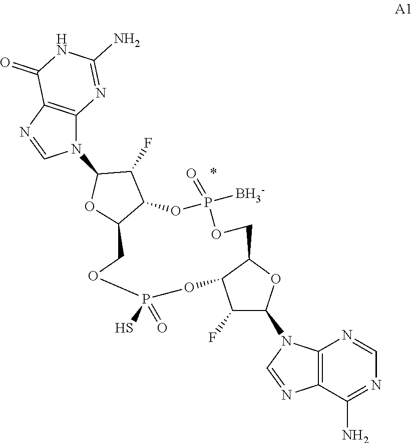

### Step 1. Synthesis of A1-b

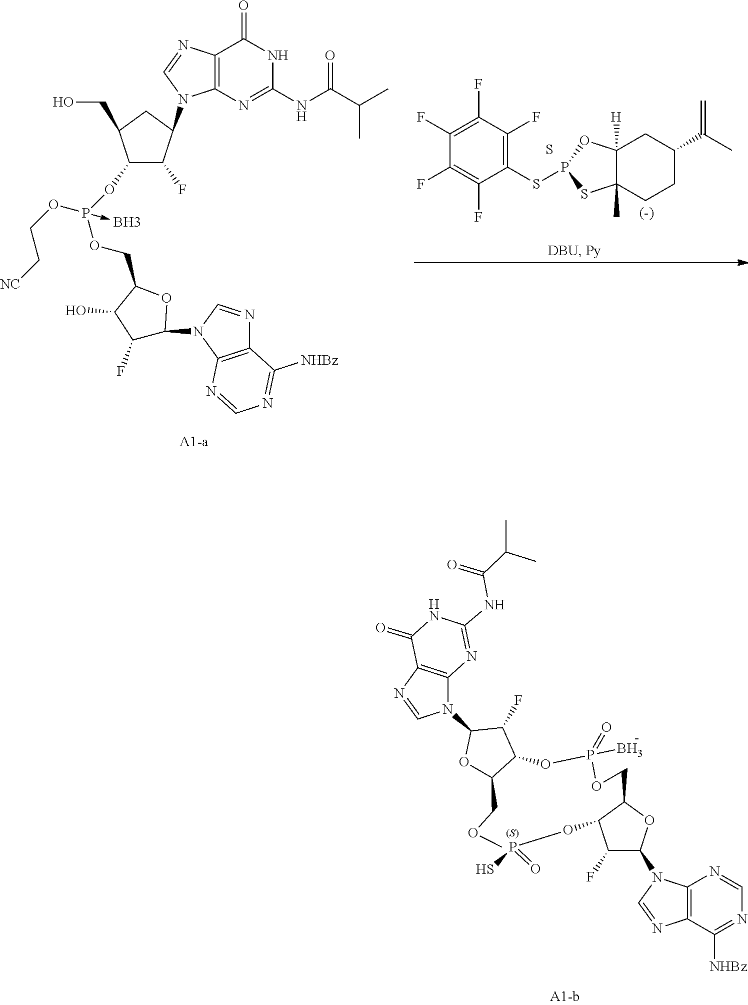

A1-a was synthesized according to the method described in WO 2019043634. Into a 100-mL 3-necked round-bottom flask purged and maintained with an inert atmosphere of argon, was placed a mixture of A1-a (1.24 g, 1.00 eq), 4A MS and DBU (3.36 g, 15.00 eq) in pyridine (20.00 mL), the flask was evacuated and flushed three times with argon. This was followed by the addition of (2S,3aS,6R,7aS)-3a-methyl-2-[(2,3,4,5,6-pentafluorophenyl) sulfanyl]-6-(prop-1-en-2-yl)-tetrahydro-4H-1,3,2lambda5-benzoxathiaphosphole-2-thione (1.97 g, 3.00 eq, in 10 mL pyridine) dropwise with stirring at room temperature. The resulting solution was stirred for 4 hours at room temperature and filtered. The filtrate was concentrated in vacuum to give 8.12 g of residue, which was used in next step without further purification. LC-MS (ES, m/z) 864.95 (M−1)⁻

### Step 2. Synthesis of A1

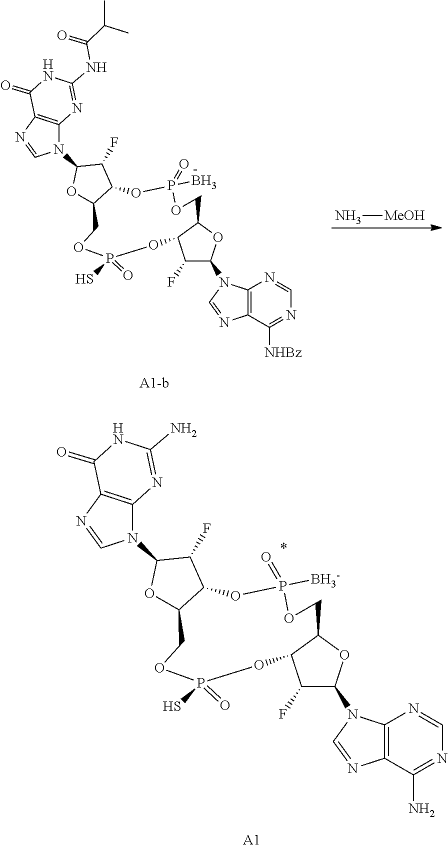

Into a 250-mL round-bottom flask, were placed A1-b (crude 8.12 g) and 4 A MS. To the above NH₃(g) in MeOH (25 mL) was added at room temperature. The resulting solution was stirred overnight at room temperature and filtered. The filtrate was concentrated in vacuo to give a residue, which was purified by HPLC to provide 60 mg of A1 (a single diastereomer). A1 was characterized to be a single diastereomer, however the absolute stereochemistry at the * position has not been resolved. LC-MS (ES, m/z) 691.00 (M−1). ⁻¹H NMR (400 MHz, Deuterium Oxide) δ 8.18 (s, 1H), 8.13 (s, 1H), 8.06 (s, 1H), 6.21 (d, J=16.0 Hz, 1H), 6.07 (d, J=14.5 Hz, 1H), 5.89 (m, 1H), 5.43-5.22 (m, 1H), 5.21-5.01 (m, 2H), 4.49-4.34 (m, 4H), 3.98 (m, 2H), 0.34 (br, 3H).

### Example 2 Synthesis of ((2R,3R,3aR,7aR,9R,10R,10aR,12S,14aR)-2-(2-amino-6-oxo-1,6-dihydro-9H-purin-9-yl)-9-(6-amino-9H-purin-9-yl)-12-((4-((4-azidobutanoyl)oxy)benzyl)thio)-3,10-difluoro-5,12-dioxidooctahydro-2H,7H-difuro[3,2-d:3′,2′-j][1,3,7,9]tetraoxa[2,8]diphosphacyclododecin-5-yl)trihydroborate (A2)

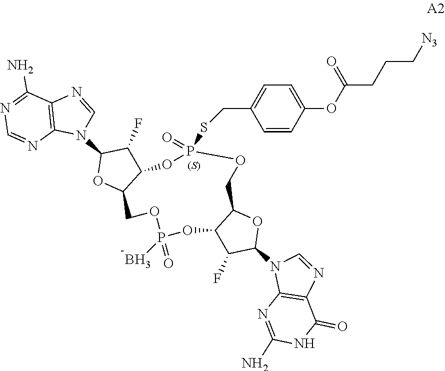

To a solution of A1 (10.00 mg, 0.014 mmol, 1.00 eq) in DMF (0.25 mL), was added 4-(iodomethyl)phenyl 4-azidobutanoate (6.00 mg, 0.017 mmol, 1.20 eq) in a mixed solvent of THF/Acetone (1:1, 0.50 mL). The resulting solution was stirred overnight at room temperature. The crude product was purified by Flash-Prep-HPLC eluting with 0-95% ACN in 10 mmol/L aq·NH4HCO3 to give 3.4 mg (27%) of A2. LC-MS (ES, m/z): [M−H]⁺ 908.05. ¹H NMR (400 MHz, Methanol-d4) δ 8.29 (s, 1H), 8.14 (s, 1H), 7.77 (s, 1H), 7.38 (m, 2H), 7.05 (m, 2H), 6.28 (m, 1H), 6.13 (m, 1H), 5.72 (s, 1H), 5.59 (s, 1H), 5.40 (br, 2H), 4.56 (m, 2H), 4.42 (m, 3H), 4.18-3.98 (m, 3H), 3.44 (m, 2H), 2.63 (m, 2H), 1.98 (m, 2H), 0.51 (br, 3H).

### Example 3 Synthesis of ((2R,3R,3aR,7aR,9R,10R,10aR,12S,14aR)-2-(2-amino-6-oxo-1,6-dihydro-9H-purin-9-yl)-9-(6-amino-9H-purin-9-yl)-12-((4-((4-(2-azidoethoxy)benzoyl)oxy)benzyl)thio)-3,10-difluoro-5,12-dioxidooctahydro-2H,7H-difuro[3,2-d:3′,2′-j][1,3,7,9]tetraoxa[2,8]diphosphacyclododecin-5-yl)trihydroborate A3

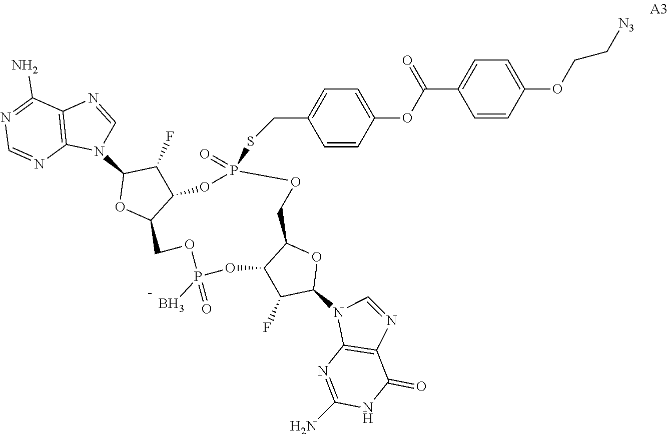

To a solution of A1 (10.00 mg, 0.014 mmol, 1.00 eq) in DMF (0.25 mL), was added 4-(iodomethyl)phenyl 4-(2-azidoethoxy)benzoate (7.35 mg, 0.017 mmol, 1.20 eq) in a mixed solvent of THF (0.25 mL) and acetone (0.25 mL). The resulting solution was stirred overnight at room temperature. The crude product was purified by Prep-HPLC eluting with acetonitrile in 5-95% 10 mmol/L aq. NH₄HCO3 to give 3.3 mg (14.42%) of A3. LC-MS [M−H]⁺ 986.10. ¹H NMR (400 MHz, Methanol-d4) δ 8.37 (s, 1H), 8.20 (s, 1H), 8.09 (d, J=8.9 Hz, 2H), 7.85 (s, 1H), 7.35 (d, J=8.2 Hz, 2H), 7.10 (t, J=8.2 Hz, 4H), 6.34 (d, J=15.9 Hz, 1H), 6.16 (d, J=20.4 Hz, 1H), 5.71 (s, 1H), 5.57 (m, 3H), 4.55 (s, 2H), 4.49-4.34 (m, 3H), 4.34-4.27 (m, 2H), 4.06 (t, J=13.5 Hz, 3H), 3.71-3.62 (m, 1H), 1.33 (s, 1H), 0.50 (br, 3H).

### Example 4 Synthesis of ((2R,3R,3aR,7aR,9R,10R,10aR,12S,14aR)-2-(2-amino-6-oxo-1,6-dihydro-9H-purin-9-yl)-9-(6-amino-9H-purin-9-yl)-12-((4-(2-(6-azidohexanamido)acetamido)benzyl)thio)-3,10-difluoro-5,12-dioxidooctahydro-2H,7H-difuro [3,2-d:3′,2′-j][1,3,7,9]tetraoxa[2,8]diphosphacyclododecin-5-yl)trihydroborate A4

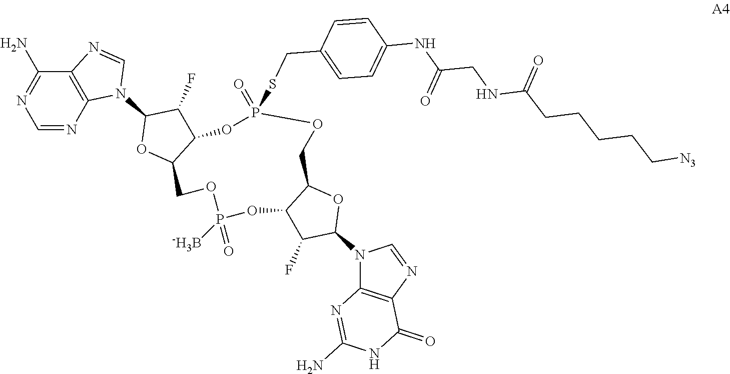

Under argon atmosphere, to a solution of A1 (100.00 mg, 0.145 mmol, 1.00 eq) in DMF (20 mL), was added dropwise 6-azido-N-([[4-(hydroxymethyl)phenyl]carbamoyl]methyl)hexanamide (50.82 mg, 0.159 mmol, 1.10 eq) in a mixed solvent of acetone (20.00 mL) and THF (20.00 mL). The resulting solution was stirred overnight at room temperature. The crude product was purified by Prep-HPLC eluting with ACN in aq. 45-85% 10 mmol/L NH₄HCO₃ to provide 35.3 mg (24.58%) of A4. LC-MS (ES, m/z): 992.15 [M−H]⁺. ¹H-NMR (400 MHz, Methanol-d4) δ 8.38 (s, 1H), 8.22 (s, 1H), 7.83 (s, 1H), 7.45 (d, J=8.3 Hz, 2H), 7.19 (d, J=8.3 Hz, 2H), 6.35 (d, J=13.1 Hz, 1H), 6.14 (d, J=20.7 Hz, 1H), 5.69 (d, J=14.9 Hz, 1H), 5.50 (s, 2H), 4.56 (s, 2H), 4.45-4.32 (m, 3H), 4.01 (s, 4H), 3.35 (m, J=1.6 Hz, 2H), 3.15 (m, J=1.7 Hz, 2H), 2.34 (t, J=7.5 Hz, 2H), 1.75-1.67 (m, 2H), 1.67-1.59 (m, 2H), 1.50-1.41 (m, 2H).

### Example 5 Synthesis of ((2R,3R,3aR,5S,7aR,9R,10R,10aR,12S,14aR)-2,9-bis(6-amino-9H-purin-9-yl)-3,10-difluoro-12-mercapto-5,12-dioxidooctahydro-2H,7H-difuro [3,2-d:3′,2′-j][1,3,7,9]tetraoxa[2,8]diphosphacyclododecin-5-yl)trihydroborate A5-I and ((2R,3R,3aR,5R,7aR,9R,10R,10aR,12S,14aR)-2,9-bis(6-amino-9H-purin-9-yl)-3,10-difluoro-12-mercapto-5,12-dioxidooctahydro-2H,7H-difuro [3,2-d:3′,2′-j][1,3,7,9]tetraoxa[2,8]diphosphacyclododecin-5-yl)trihydroborate A5-II

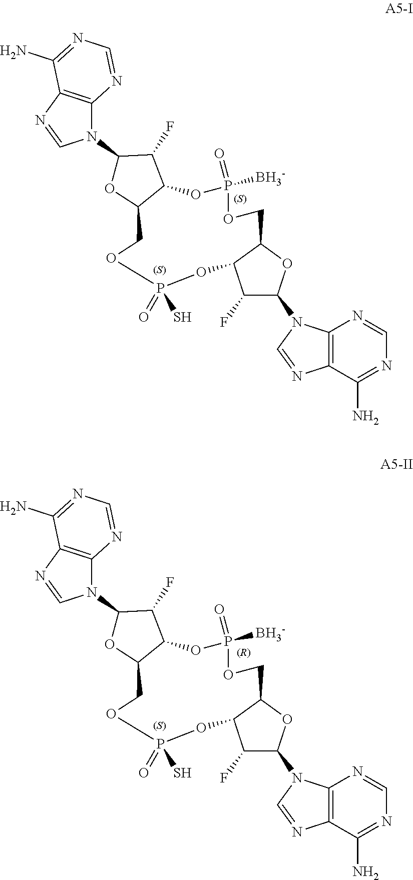

### Step 1. Synthesis of ((2R,3R,3aR,7aR,9R,10R,10aR,12S,14aR)-2,9-bis(6-benzamido-9H-purin-9-yl)-3,10-difluoro-12-mercapto-5,12-dioxidooctahydro-2H,7H-difuro[3,2-d:3′,2′-j][1,3,7,9]tetraoxa[2,8]diphosphacyclododecin-5-yl)trihydroborate A5-b

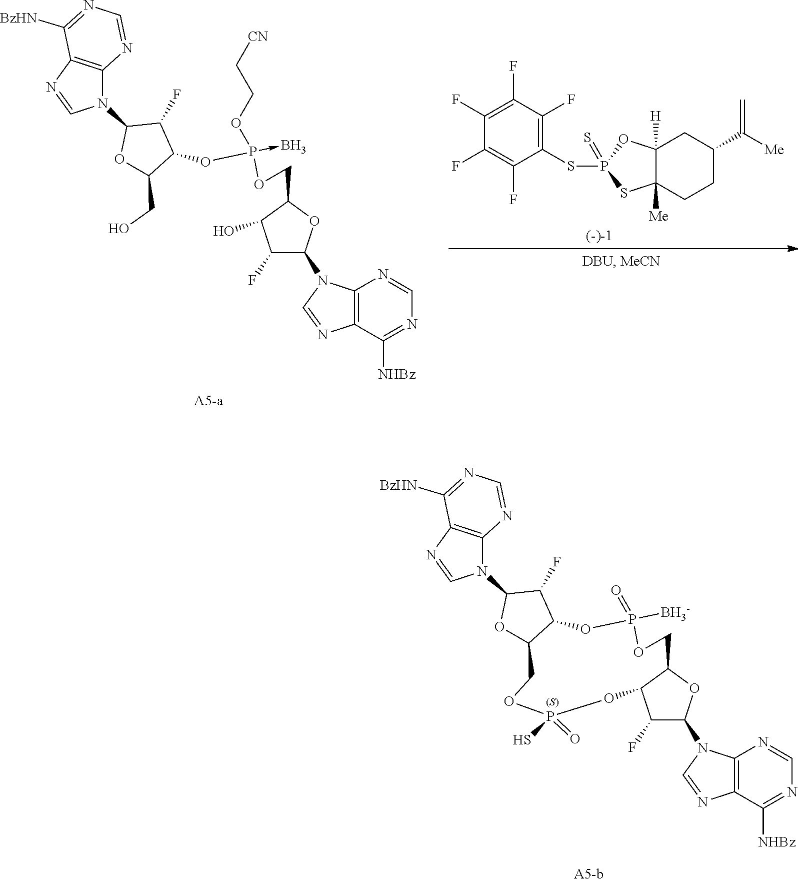

A5-a was synthesized according to the synthetic method described in WO 2019043634. (2 S,3 aS,6R,7aS)-3a-methyl-2-((perfluorophenyl)thio)-6-(prop-1-en-2-yl)hexahydrobenzo[d][1,3,2]oxathiaphosphole 2-sulfide (779 mg, 1.75 mmol, 1.50 eq) and A5-a ((1.00 g, 1.16 mmol, 1.00 eq) were co-evaporated with ACN (20.0 mL×3) respectively. A5-a (1.00 g, 1.16 mmol, 1.00 eq) was dissolved in pyridine (400 mL), DBU (2.66 g, 17.45 mmol, 2.63 mL, 15 eq) was added at 25° C., followed by (2S,3aS,6R,7aS)-3a-methyl-2-((perfluorophenyl)thio)-6-(prop-1-en-2-yl)hexahydrobenzo[d][1,3,2]oxathiaphosphole 2-sulfide (779 mg, 1.75 mmol, 1.50 eq). The reaction mixture was stirred at 25° C. for 1 hr and concentrated to give a residue, which was precipitated in MTBE (100 mL×3). The solid was collected and then dissolved in MeOH, after filtration, the filtrate was added into the TEA-resin to remove DBU. After concentration, A5-b (0.980 g, crude) was obtained. LC-MS [M−H]⁺ 883.2.

### Step 2. Synthesis of A5-I and A5-II

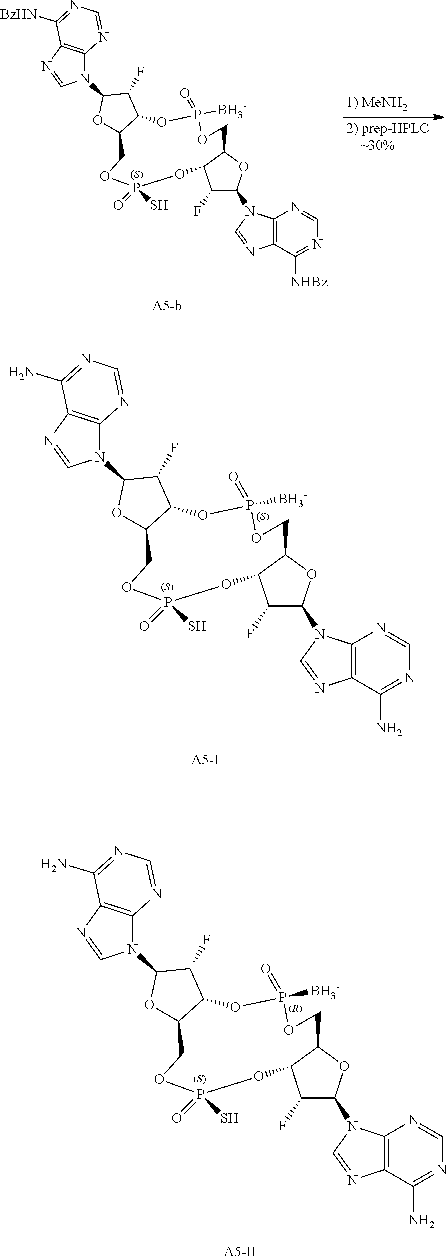

The mixture of MeNH₂ (115 mg, 1.11 mmol, 20.0 mL, 30% purity, 1.00 eq) and A5-b (0.980 g, 1.11 mmol, 1.00 eq) was stirred at 25° C. for 2 hrs. The mixture was concentrated to give a residue (846 mg, crude), which was purified by prep-HPLC (column: Phenomenex Gemini-NX C18 150×30 mm, 5 μM; mobile phase: [0.1M TEAB-ACN]; B %: 5%-35%, 10 min) to provide compound A5-II (0.154 mg) and compound A5-I (0.105 mg).

A5-I: LC-MS [M−H]⁺ 675.1. RT=1.067 min. ¹H NMR (400 MHz, D₂O) δ ppm (br, 3H), 0.80-0.99 (t, J=7.2 Hz, 18H), 2.81 (q, J=7.2 Hz, 12H), 3.59-3.86 (m, 2H), 4.03-4.28 (m, 4H), 4.58-4.75 (m, 1H), 4.77-4.94 (m, 1H), 5.07-5.31 (m, 1H), 5.48-5.70 (m, 1H), 5.96 (br t, J=15.6 Hz, 2H), 7.58 (s, 1H) 7.75 (s, 1H), 7.93 (s, 1H), 8.04 (s, 1H).

A5-II: LC-MS [M−H]⁺ 675.1 RT=1.163 min ¹H NMR (400 MHz, D₂O) δ ppm-0.18-0.64 (m, 3H) 1.23 (t, J=7.2 Hz, 18H) 3.15 (q, J=7.2 Hz, 12H) 4.03-4.13 (m, 2H) 4.28-4.45 (m, 2H) 4.30-4.46 (m, 1H) 4.47-4.58 (m, 1H) 4.47-4.58 (m, 2H) 4.83-5.12 (m, 2H) 5.39-6.03 (m, 2H) 6.41 (dd, J=16.0, 6.00 Hz, 2H) 8.06 (d, J=3.6 Hz, 2H) 8.33-8.55 (m, 2H).

### Example 6 Synthesis of A6-I and A6-II

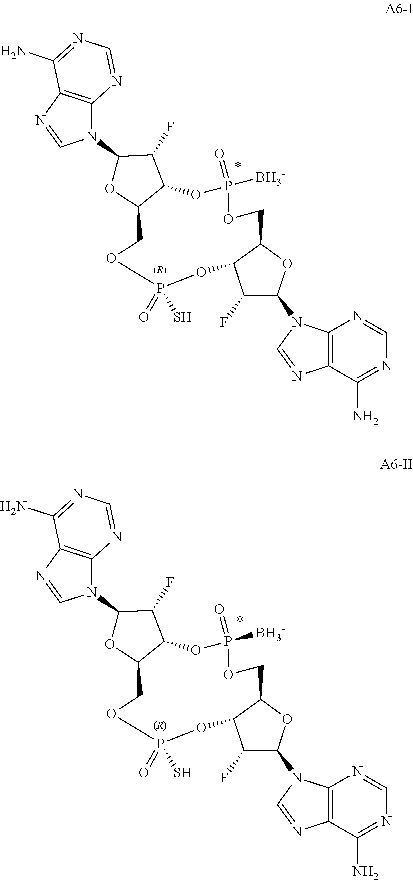

### Step 1. Synthesis of A6-a and A6-b

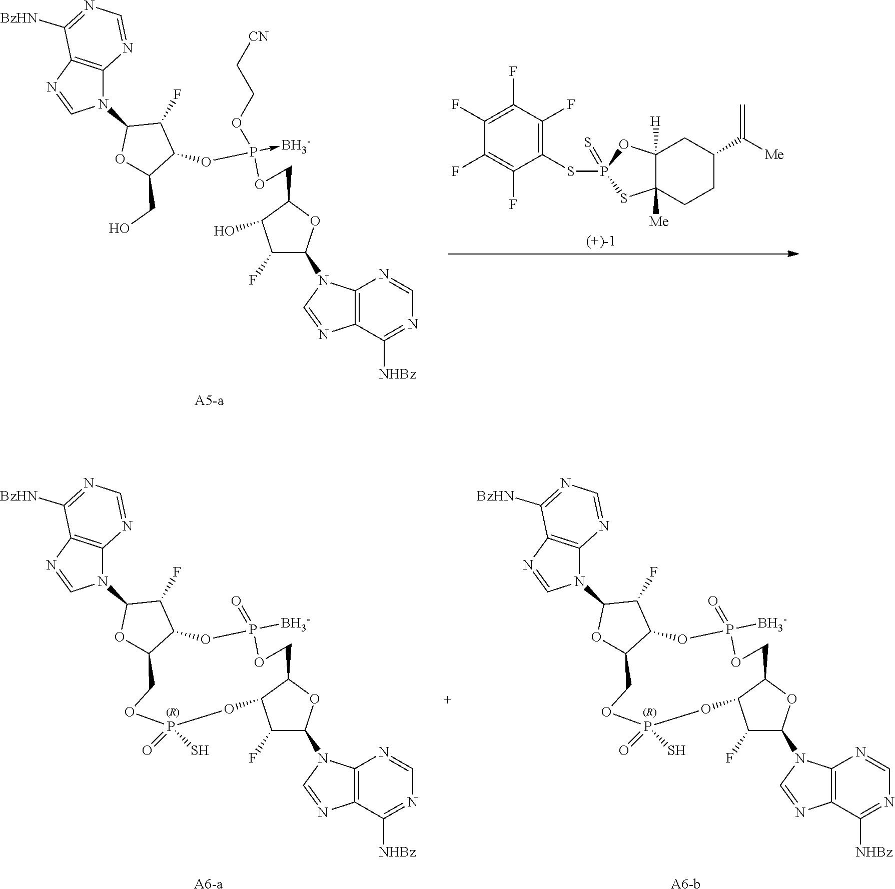

A5-a was synthesized according to the synthetic method described in WO 2019043634. A5-a (1.00 g, 1.16 mmol, 1.00 eq) and (2R,3aS,6R,7aS)-3a-methyl-2-((perfluorophenyl)thio)-6-(prop-1-en-2-yl)hexahydrobenzo[d][1,3,2]oxathiaphosphole 2-sulfide (779 mg, 1.75 mmol, 1.50 eq) were evaporated in ACN (20.0 mL×3) respectively. To a solution of A5-a (1.00 g, 1.16 mmol, 1.00 eq) and DBU (1.77 g, 11.6 mmol, 1.75 mL, 10.0 eq) in ACN (40.0 mL), was added a solution of (2R,3aS,6R,7aS)-3a-methyl-2-((perfluorophenyl)thio)-6-(prop-1-en-2-yl)hexahydrobenzo[d][1,3,2]oxathiaphosphole 2-sulfide (779 mg, 1.75 mmol, 1.50 eq) in ACN (10 mL) dropwise at 0° C. for 5 min, then the mixture was stirred at 0° C. for 2 hrs. The reaction mixture was concentrated to give a residue, which was dissolved in DCM (10.0 mL), triturated with TBME (100 mL) at 25° C. for 10 min for 3 times. After filtration, the residue was purified by prep-HPLC (column: YMC-Triart Prep C18 250×50 mm, 10 μM; mobile phase: [0.1M TEAB-ACN]; B %: 10%-30%,22 min) to provide compound A-6a (350 mg, crude) and compound A6-b (80 mg, crude).

A6-a: LC-MS [M−H]⁺883.2. RT=1.039 min.

A6-b LC-MS [M−H]⁺883.2. RT=1.069 min.

### Step 2. Synthesis of A6-I and A6-II

Following the procedure as described in step 2 of Example 4, A6-I and A6-II were synthesized from A6-a and A6-b respectively and characterized to be a single diastereomer. However, the absolute stereochemistry at the * position has not been resolved.

A6-I LC-MS [M−H]⁺=675.1. ¹H NMR (400 MHz, D2O) δ ppm 0.25 (br, 3H), 1.25 (m, 18H), 3.18 (q, J=7.2 Hz, 12H), 3.36 (q, J=7.2 Hz, 1H), 3.89-4.21 (m, 2H), 4.36 (br d, J=12.4 Hz, 1H), 4.46-4.61 (m, 3H), 4.89-5.14 (m, 1H), 5.39-5.75 (m, 2H), 6.41 (dd, J=18.4, 15.6 Hz, 2H), 7.88 (s, 1H), 8.12 (s, 1H), 8.31 (d, J=2.8 Hz, 2H).

A6-II LC-MS [M−H]⁺=675.1. ¹H NMR (400 MHz, D2O) δ ppm 0.39 (br d, J=14.4 Hz, 3H), 1.26 (t, J=7.2 Hz, 18H), 3.18 (q, J=7.2 Hz, 12H), 4.05 (br dd, J=12.0, 6.17 Hz, 2H), 4.40-4.64 (m, 4H), 5.23 (br s, 2H), 5.46-5.71 (m, 2H), 6.29 (br d, J=12.0 Hz, 1H), 6.19-6.40 (m, 1H), 8.00 (br d, J=7.2 Hz, 2H), 8.15-8.34 (m, 2H).

### Example 7 Synthesis of ((2R,3R,3aR,5S,7aR,9R,10R,10aR,12S,14aR)-2,9-bis(6-amino-9H-purin-9-yl)-12-((4-((4-(2-azidoethoxy)benzoyl)oxy)benzyl)thio)-3,10-difluoro-5,12-dioxidooctahydro-2H,7H-difuro [3,2-d:3′,2′-j][1,3,7,9]tetraoxa[2,8]diphosphacyclododecin-5-yl)trihydroborate A7

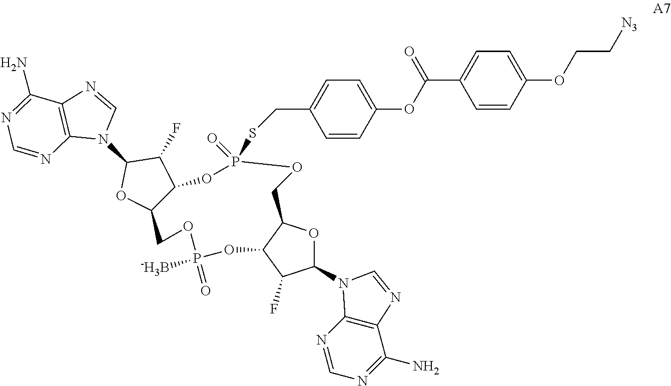

SOCl₂ (53.2 mg, 447 umol, 32.4 uL, 2.00 eq) was added dropwise to a solution of I-2i (70.0 mg, 223 umol, 1 eq) in DCM (2.00 mL) at 0° C. The mixture was stirred at 0° C. for 2 hrs. The solution was quenched with aq. NaHCO₃ (50.0 mL), extracted with DCM (30 mL×2), the combined organic layers were dried with Na₂SO₄, filtered and concentrated to give a light yellow solid, which was dissolved in a mixed solvent of acetone (1.00 mL) and THF (1.00 mL), potassium iodide (148 mg, 892 umol, 4.00 eq) was then added to the solution and the resulting mixture was stirred at 25° C. for 1 hour, to which was then added a solution of A5-I (100 mg, 148 umol, 1.00 eq) in DMF (0.500 mL) and stirred at 25° C. for 12 hrs. The reaction mixture was purified by prep-HPLC (neutral condition) eluting with 10-50% ACN in 10 mM NH₄HCO₃ to compound A7 (52.0 mg, 52.4 umol, 36.4% yield, 97.9% purity). RT=1.085 min. LC-MS [M−H]⁺=970.3. ¹H NMR (400 MHz, DMSO-d₆) 8.56 (s, 1H), 8.23-8.17 (m, 2H), 8.05-7.99 (m, 3H), 7.26-7.24 (br d, J=8.4 Hz, 2H), 7.14-7.08 (br dd, J=8.4, 15.2 Hz, 4H), 6.41-6.30 (m, 2H), 5.87-5.74 (m, 1H), 5.67-5.53 (m, 1H), 5.51-5.46 (m, 2H), 4.47-4.42 (br d, J=19.2 Hz, 2H), 4.35-4.32 (m, 4H), 4.29-4.27 (m, 1H), 3.97-3.70 (m, 3H), 3.69-3.68 (br t, J=4.4 Hz, 2H), 0.23 (br s, 3H).

### Example 8 Synthesis of ((2R,3R,3aR,5S,7aR,9R,10R,10aR,12S,14aR)-2,9-bis(6-amino-9H-purin-9-yl)-12-((4-(2-(6-azidohexanamido)acetamido)benzyl)thio)-3,10-difluoro-5,12-dioxidooctahydro-2H,7H-difuro [3,2-d:3′,2′-j][1,3,7,9]tetraoxa[2,8]diphosphacyclododecin-5-yl)trihydroborate A8

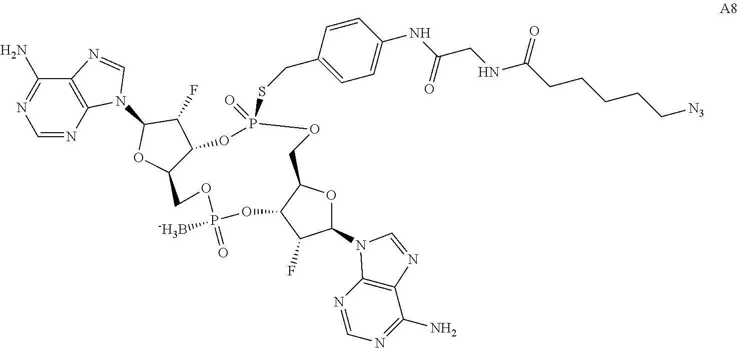

A8 was synthesized according to the synthetic method as described in Example 4 starting from I-11 and A5-I. LC-MS (ES, m/z): 976.4 [M−H]⁺. HPLC purity 94%. ¹H NMR (400 MHz, DMSO-d₆) δ=9.97 (s, 1H), 8.51 (s, 1H), 8.21 (s, 1H), 8.17 (s, 1H), 8.03 (s, 1H), 7.44-7.42 (d, J=8.0 Hz, 2H), 7.13-7.11 (d, J=8.4 Hz, 2H), 6.40-6.28 (m, 2H), 5.94-5.79 (m, 1H), 5.62-5.39 (m, 3H), 4.46-4.43 (m, 2H), 4.34-4.26 (m, 2H), 4.16-4.13 (m, 1H), 3.87-3.77 (m, 5H), 3.31-3.27 (t, J=6.8 Hz, 2H), 2.17-2.13 (t, J=7.6 Hz, 2H), 1.55-1.48 (quin, J=7.0 Hz, 4H), 1.34-1.27 (m, 2H), 0.25 (br s, 3H). two drops of D₂O were added to the DMSO sample in order to decrease the influence of active hydrogens during NMR test.

### Example 9 Synthesis of ((2R,3R,3aR,5S,7aR,9R,10R,10aR,12S,14aR)-2,9-bis(6-amino-9H-purin-9-yl)-12-((4-(2-(2-(2-azidoethoxy)ethoxy)ethoxy)benzyl)thio)-3,10-difluoro-5,12-dioxidooctahydro-2H,7H-difuro[3,2-d:3′,2′-j][1,3,7,9]tetraoxa[2,8]diphosphacyclododecin-5-yl)trihydroborate A9

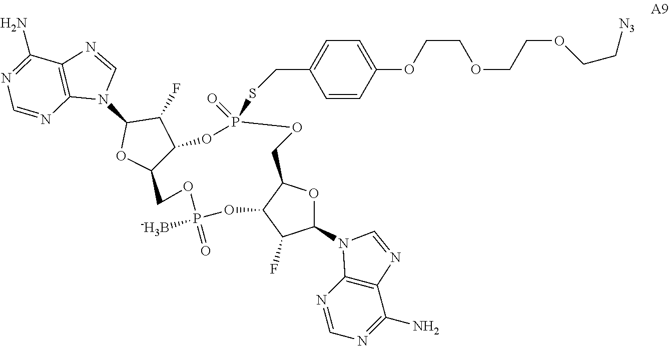

A9 is synthesized according to the synthetic method as described in Example 4 starting from I-12 and A5-I.

### Example 10 Synthesis of (2R,3R,3aR,5S,7aR,9R,10R,10aR,14aR)-2,9-bis(6-amino-9H-purin-9-yl)-5-((4-(2-(2-(2-azidoethoxy)ethoxy)ethoxy)benzyl)thio)-3,10-difluoro-12-hydroxyoctahydro-2H,7H-difuro [3,2-d:3′,2′-j][1,3,7,9]tetraoxa[2,8]diphosphacyclododecine 5,12-dioxide A10

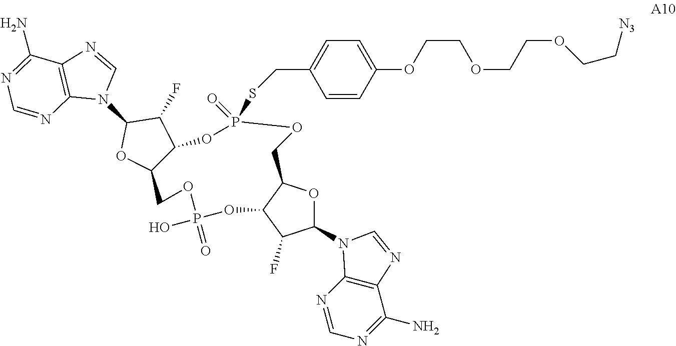 [SMILES: [N-]=[N+]=NCCOCCOCCOc1ccc(CS[P@@]2(=O)OC[C@H]3O[C@@H](n4cnc5c(N)ncnc54)[C@H](F)[C@@H]3OP(=O)(O)OC[C@H]3O[C@@H](n4cnc5c(N)ncnc54)[C@H](F)[C@@H]3O2)cc1]

A10 is synthesized according to the synthetic method as described in Example 4 starting from 1-12 and (2R,3R,3aR,7aR,9R,10R,10aR,12S,14aR)-2,9-bis(6-amino-9H-purin-9-yl)-3,10-difluoro-5-hydroxy-12-mercaptooctahydro-2H,7H-difuro [3,2-d:3′,2′-j][1,3,7,9]tetraoxa[2,8]diphosphacyclododecine 5,12-dioxide, which can be obtained following the procedure as described in WO 2018200812 A1.

In the following examples, CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′, connecting at either 5′-O or/and 3′-O of the terminal nucleotide.

### Example 11 Synthesis of A11

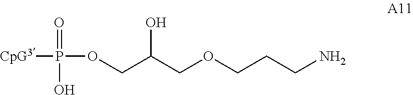

A11 was synthesized by Integrated DNA Technologies (IDT), using 3′ amino modifier (Mod Code: 3AmMO) connecting at 3′-O of the terminal nucleotide. Purity 97%. Calculated mass 9883 Da, measured mass 9882 Da.

### Example 12 Synthesis of A12

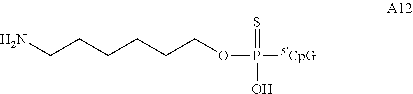

A12 was synthesized by Integrated DNA Technologies (IDT), using 5′ amino modifier C6 (Mod Code: 5AmMC6) connecting at 5′-O of the terminal nucleotide. Purity 97%. Calculated mass 9867 Da, measured mass 9866 Da.

### Example 13 Synthesis of A13

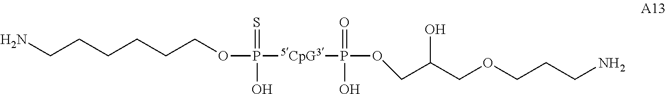

A-13 was synthesized by Integrated DNA Technologies (IDT), using 3′ amino modifier (Mod Code: 3AmMO) connecting at 3′-O of the terminal nucleotide and 5′ amino modifier C6 (Mod Code: 5AmMC6) connecting at 5′-O of the terminal nucleotide. Purity 97%. Calculated mass 10078 Da, measured mass 10077 Da.

### Example 14 Synthesis of A14

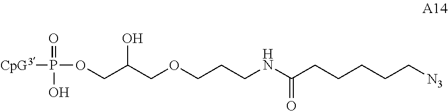

A-14 was synthesized by Integrated DNA Technologies (IDT), using 3′ azide modifier (Mod Code: 3AzideN) connecting at 3′-O of the terminal nucleotide. Purity 97%. Calculated mass 10022 Da, measured mass 10020 Da.

### Example 15 Synthesis of A15

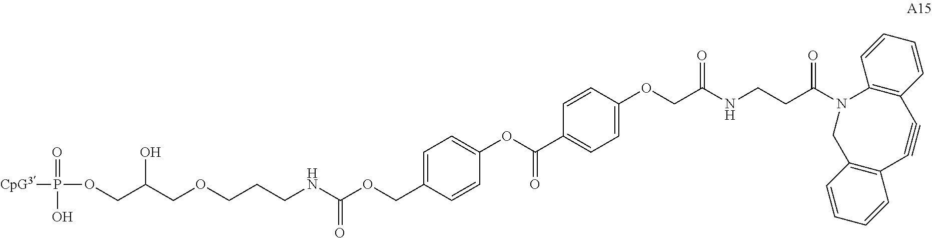

Route A: A11 (2.8 mL, 20 mg) in PBS, pH 8 was charged to a 15 mL Falcon tube and I-5 solution (2.74 mL, 7.1 mg, 5 eq.) in DMF (3.0 mL) was added. The resulting reaction was mixed and incubated at 22° C. for 2 h. The crude reaction was purified by prep-HPLC (column: Phenomenex Luna C18 150×21.2 mm, 5 μM; mobile phase: [100 mM triethylammonium acetate, pH 7.0—ACN]; B %: 5-70%, 25 min). Fractions containing product were combined and the volatiles were evaporated in vacuo. The material was then buffer exchanged into PBS, pH 7.4 via UF/DF using a Vivaspin20 concentrator (PES, 3 kDa MWCO) to give a solution of A15 at 5.68 mg/mL (11.1 mg, 56%). Analysis was performed by HPLC with UV detection at 260 nm (column: ACE Excel Super C18 7.5×2.1 mm, 5 μM; mobile phase: [0.1 M triethylammonium acetate, pH 7.4—ACN]; B %: 0-100%, 10 min). RT=5.4 min. Calculated mass 10469 Da, measured mass 10469 Da.

Route B: A11 (40.0 mg, 4.05 umol, 1.00 eq) was dissolved in PBS (2 mL, pH=8). To this solution was added 1-13 (34.0 mg, 46.8 umol, 11.5 eq) in DMF (5 mL) at 20° C. The resulting yellow solution was stirred at 20° C. for 16 h. After the crude mixture was concentrated, the residue was washed with water (5 mL). The filtrate was concentrated under oil pump to give A15 as yellow oil (50 mg, crude), which was used directly for the next step. Analysis was performed by HPLC with UV detection at 260 nm (column: X-bridge Shield RP18 50×2.1 mm, 5 μM; mobile phase: [water (10 mM NH₄HCO₃)-ACN]; B %: 0-60%, 4.5 min). RT=1.70 min. Calculated mass 10469 Da, measured mass 10469 Da.

### Example 16 Synthesis of A16 (a Mixture of Regioisomers as Shown Below)

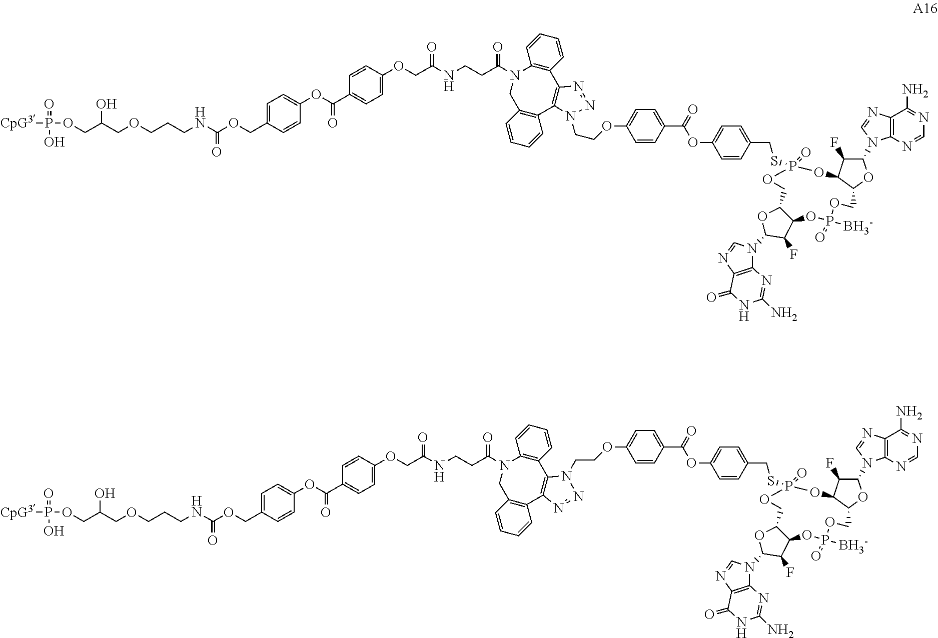

To A15 (7.9 mg) in PBS, pH 7.4 (1.39 mL) was added A3 (3.0 mg, 4 eq.) in a 1:1 solution of PBS, pH 7.4/DMSO (298 μL). The resulting reaction was mixed and incubated at 22° C. for 2 h. The crude reaction was buffer exchanged into 20 mM ammonium bicarbonate, pH 7.0 via UF/DF using a Vivaspin20 concentrator (PES, 3 kDa MWCO). The material was subsequently purified by prep-HPLC (column: Phenomenex Luna C18 150×21.2 mm, 5 μM; mobile phase: [100 mM triethylammonium acetate, pH 7.0—ACN]; B %: 5-70%, 25 min). Fractions containing product were combined and the volatiles were evaporated in vacuo. The material was then buffer exchanged into 20 mM ammonium bicarbonate, pH 7.0 via UF/DF using a Vivaspin20 concentrator (PES, 3 kDa MWCO). The resulting solution was lyophilized to give a mixture of regioisomers A16 (3.4 mg, 43%). Analysis was performed by HPLC (column: ACE Excel Super C18 7.5×2.1 mm, 5 μM; mobile phase: [0.1 M triethylammonium acetate, pH 7.4—ACN]; B %: 0-100%, 10 min). Purity 99%. RT=5.5 min. Calculated mass 11455 Da, measured mass 11456 Da.

### Example 17 Synthesis of A17 (a Mixture of Regioisomers as Shown Below)

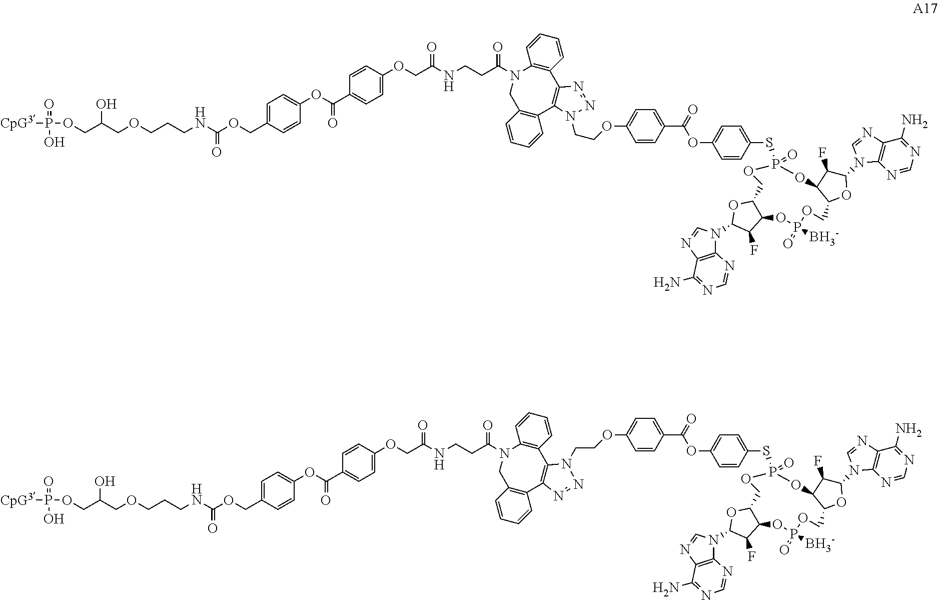

A mixture of regioisomers A17 was synthesized by the reaction of A15 with A7 according to the procedure described in Example 16. The material was purified by prep-HPLC (column: Clarity 150×21.2 mm, 5 μM; mobile phase: [100 mM triethylammonium acetate, pH 7.0—ACN]; B %: 10-40%, 10 min). Analysis was performed by HPLC with UV detection at 260 nm (column: Clarity RP C18 100×4.6 mm, 3 μM; mobile phase: [100 mM TEAA—90% ACN/10% 100 mM TEAA]; B %: 10-50%, 30 min). Purity 95%. RT=18.21 min. Calculated mass 11439 Da, measured mass 11439 Da.

### Example 18 Synthesis of A18

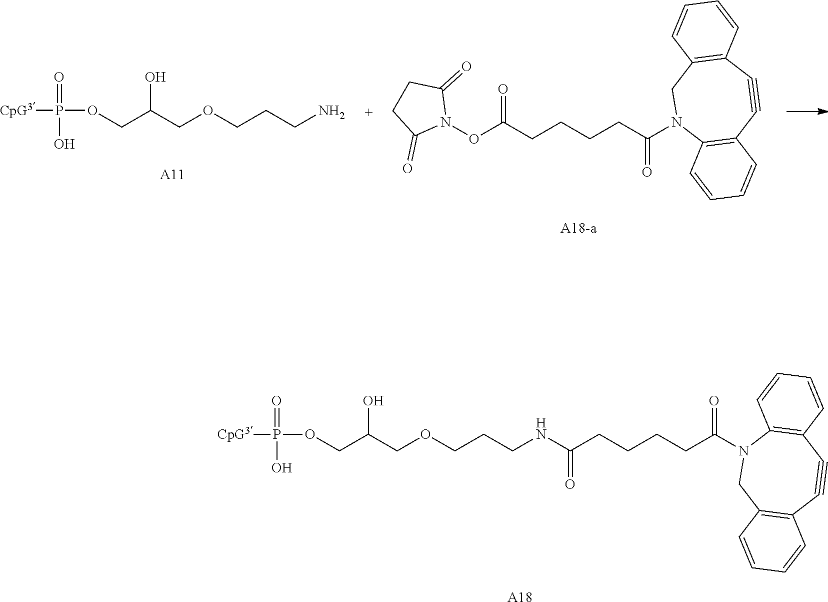

A18-a (104 mg, 242 μmop was dissolved in DMF (2 mL). To this solution was added A11 (60.0 mg, 6.07 μmop in PBS (1 mL, pH=8). The reaction mixture was stirred at ° C. for 16 h. After the crude mixture was concentrated and washed with water, the filtrate was lyophilized to give A18 (79 mg, crude), which was used directly for the next reaction. Analysis was performed by HPLC (column: X-bridge Shield RP18 50×2.1 mm, 5 μM; mobile phase: [water (10 mM NH₄HCO3)-ACN]; B %: 0%-60%, 4.5 min). RT=1.36 min. Calculated mass 10198 Da, measured mass 10198 Da.

### Example 19 Synthesis of A19 (a Mixture of Regioisomers as Shown Below)

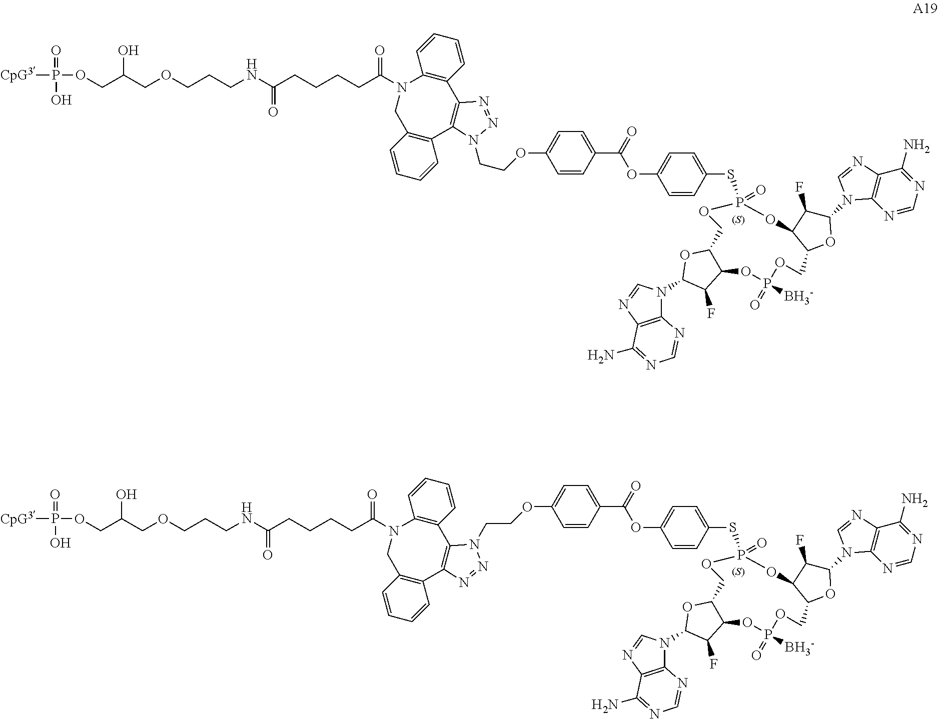

A18 (15.0 mg, 1.47 mol, 1.00 eq) was dissolved in PBS, pH 7.4 (0.2 mL). To this solution was added A7 (7.15 mg, 7.36 mol, 5.00 eq) dissolved in a 1:1 solution of PBS, pH 7.4/DMSO (0.2 mL). The resulting reaction was mixed and incubated at 20° C. for 16 h. The crude mixture was purified by prep-HPLC (column: YMC Triart C18 150×25 mm, 5 μM, mobile phase: [0.1M TEAA-ACN]; B %: 5%-20%, 7 min). The fractions containing the product were combined and lyophilized to give a mixture of regioisomers A19 (1.7 mg). Analysis was performed by HPLC (column: Clarity RP C18 100×4.6 mm, 3 μM; mobile phase: [100 mM TEAA— 90% ACN/10% 100 mM TEAA]; B %: 10-50%, 30 min). Purity 86%. RT=10.16 min. Calculated mass 11169 Da, measured mass 11169 Da.

### Example 20 Synthesis of A20

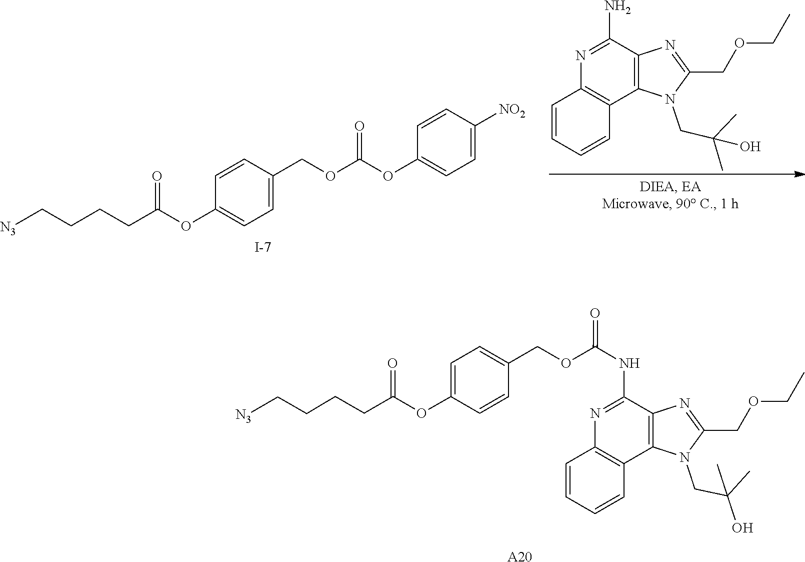

To a mixture of 1-7 (245 mg, 0.59 mmol, 1.5 eq) in dry ethyl acetate (8 mL) were added 1-(4-amino-2-(ethoxymethyl)-1H-imidazo[4,5-c]quinolin-1-yl)-2-methylpropan-2-ol (124 mg, 0.40 mmol, 1.0 eq) and N,N-diisopropylethylamine (127 mg, 0.98 mmol. 2.5 eq). The mixture was microwaved at 95° C. for 1 h under nitrogen atmosphere. Then the solution was cooled to rt, extracted with ethyl acetate (3×20 mL) and the combined organic layers were washed with water (3×40 mL). The combined organic layers were dried over magnesium sulfate, filtered and concentrated. The residue was purified by prep-TLC (petroleum ether: ethyl acetate=5:1) to give A20 (105 mg, 45%). LC-MS (ESI, m/z): 590 [M+H]⁺. ¹H NMR (400 MHz, DMSO): δ 8.64-8.62 (d, J=7.6 Hz, 1H), 8.15-8.13 (d, J=7.6 Hz, 1H), 7.73 (m, 1H), 7.65-7.64 (m, 1H), 7.53-7.51 (d, J=8.0 Hz, 2H), 7.15-7.13 (d, J=8.4 Hz, 2H), 5.29 (s, 2H), 4.76 (m, 2H), 3.53-3.52 (m, 6H), 3.38-3.36 (m, 2H), 2.61-2.59 (m, 2H), 1.65-1.62 (m, 4H) and 1.16-1.10 (m, 9H).

### Example 21 Synthesis of A21

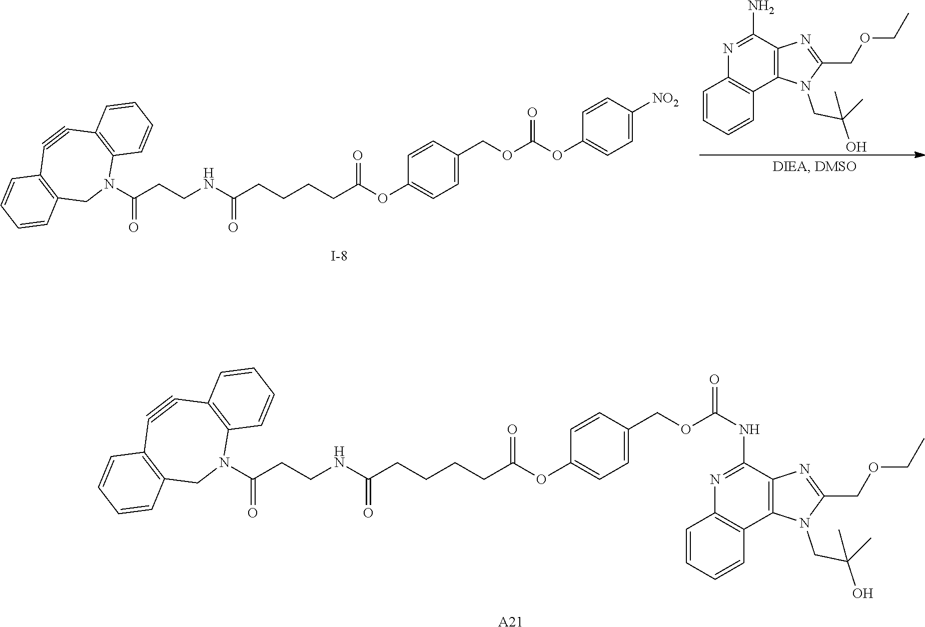

To a solution of I-8 (39 mg, 0.058 mmol) and 1-(4-amino-2-(ethoxymethyl)-1H-imidazo[4,5-c]quinolin-1-yl)-2-methylpropan-2-ol (20 mg, 0.064 mmol) in DMSO (1 mL) was added DIEA (22 mg, 0.17 mmol). The mixture was stirred at rt for 16 h. After removal of solvent, the mixture was purified by prep-HPLC to give A21 (9.0 mg, 18%). LC-MS (ESI, m/z): 851 [M+H]⁺. ¹HNMR (400 MHz, CDCl₃): δ 8.51-8.54 (d, 1H), 7.99-8.02 (d, 1H), 7.13-7.66 (m, 14H), 6.66 (m, 1H), 5.30 (s, 2H), 5.14-5.16 (d, 1H), 4.92 (s, 2H), 3.14-3.74 (m, 7H), 1.59-2.05 (m, 10H), 1.34 (s, 6H), 1.16 (t, 3H).

### Example 22 Synthesis of A22

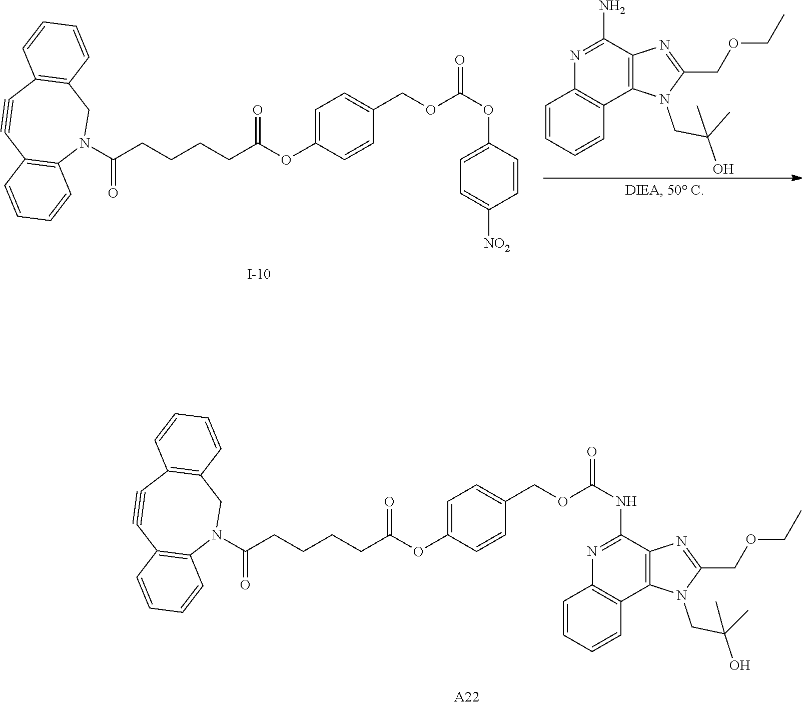

To a mixture of 1-10 (90 mg, 0.15 mmol) and 1-(4-amino-2-(ethoxymethyl)-1H-imidazo[4,5-c]quinolin-1-yl)-2-methylpropan-2-ol (63 mg, 0.18 mmol) in dioxane (2 mL) was added DIEA (58 mg, 0.45 mmol). The mixture was stirred at 50° C. for 6 h. After removal of solvent, the mixture was purified by prep-HPLC to provide A22 (58 mg, 38%). LC-MS (ESI, m/z): 780 [M+H]⁺. ¹HNMR (300 MHz, CDCl₃): δ 8.10-8.16 (m, 2H), 7.66-7.70 (d, 1H), 7.57-7.63 (t, 1H), 7.22-7.49 (m, 10H), 5.29 (s, 2H), 5.14-5.18 (d, 1H), 4.89 (s, 2H), 4.78 (s, 2H), 3.60-3.68 (m, 3H), 2.23-2.37 (m, 4H), 1.92-2.08 (m, 4H), 1.45-1.54 (m, 4H), 1.32 (s, 6H), 1.21-1.26 (t, 3H).

### Example 23 Synthesis of A23 (a Mixture of Regioisomers as Shown Below)

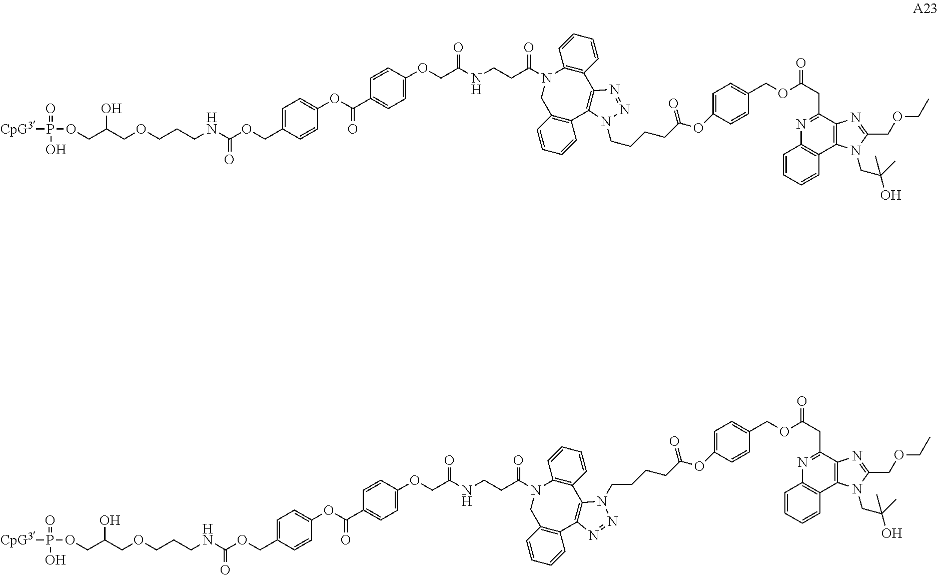

A mixture of regioisomers A23 was synthesized by the reaction of A15 with A20 according to the procedure described in Example 16. Analysis was performed by HPLC (column: Clarity RP C18 100×4.6 mm, 3 μM; mobile phase: [100 mM TEAA— 90% ACN/10% 100 mM TEAA]; B %: 10-50%, 30 min). Purity 98%. RT=19.45 min. Calculated mass 11058 Da, measured mass 11059 Da.

### Example 24 Synthesis of A24 (a Mixture of Regioisomers as Shown Below)

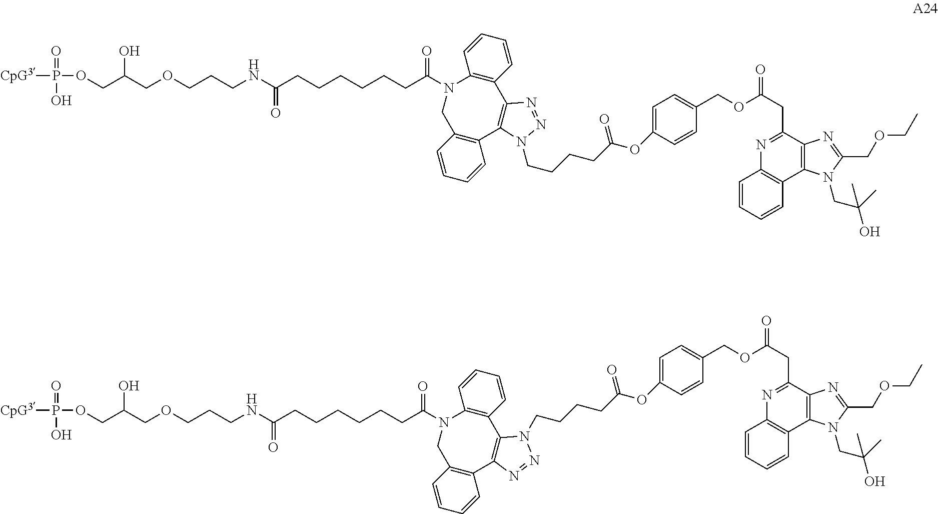

A mixture of regioisomers A24 was synthesized by the reaction of A18 with A20 according to the procedure described in Example 16. Analysis was performed by HPLC (column: Clarity RP C18 100×4.6 mm, 3 μM; mobile phase: [100 mM TEAA— 90% ACN/10% 100 mM TEAA]; B %: 10-50%, 30 min). Purity 91%. RT=17.25 min. Calculated mass 10787 Da, measured mass 10788 Da.

### Example 25 Synthesis of A25 (a Mixture of Regioisomers as Shown Below)

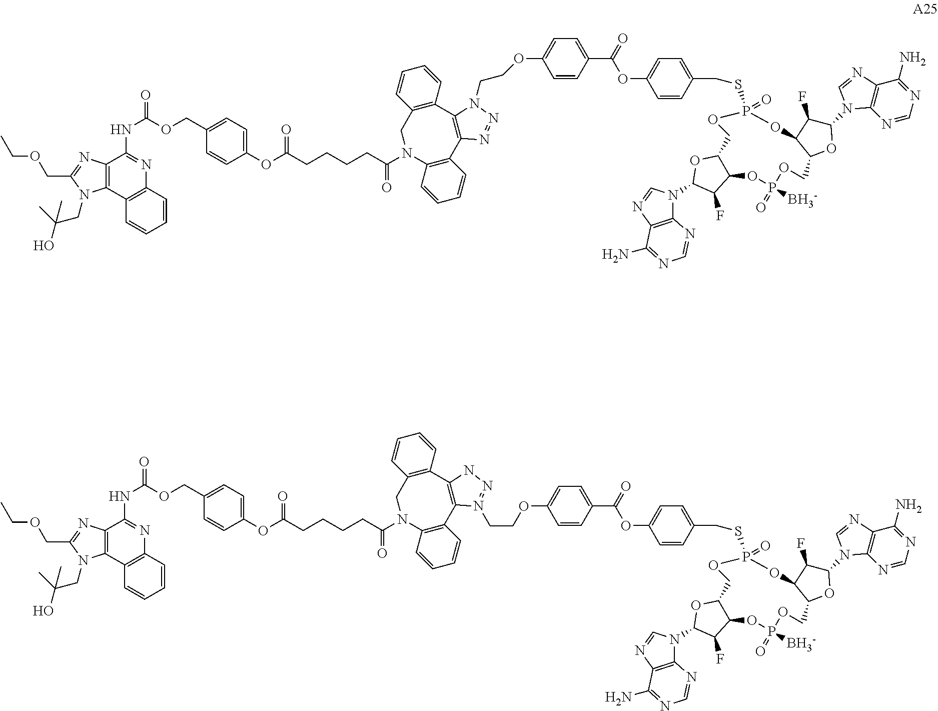

A22 (7.0 mg, 8.98 umol) was dissolved in PBS, pH 7.4 (0.5 mL). To this solution was added A7 (10.4 mg, 10.7 umol) in a 1:1 solution of PBS, pH 7.4/DMF (0.5 mL). The resulting reaction was mixed and incubated at 20° C. for 16 h. The LC-MS showed that A22 was consumed completely. The crude mixture was purified by prep-HPLC (column: Phenomenex Gemini-NX C18 75×30 mm, 3 μM; mobile phase: [water (10 mM NH₄HCO₃)-ACN]; B %: 30%-60%, 10 min). The fractions containing the product were combined and lyophilized to give a mixture of regioisomers A25 (9.0 mg, 54% yield). Analysis was performed by HPLC (column: X-bridge Shield RP18 50×2.1 mm, 5 μM; mobile phase: [water (10 mM NH₄HCO₃)-ACN]; B %: 10-80%, 4.5 min). Purity 99%. RT=2.50 min. Calculated mass 1752 Da, measured mass 1752 Da.

### Example 26 Synthesis of A26 (a Mixture of Regioisomers as Shown Below)

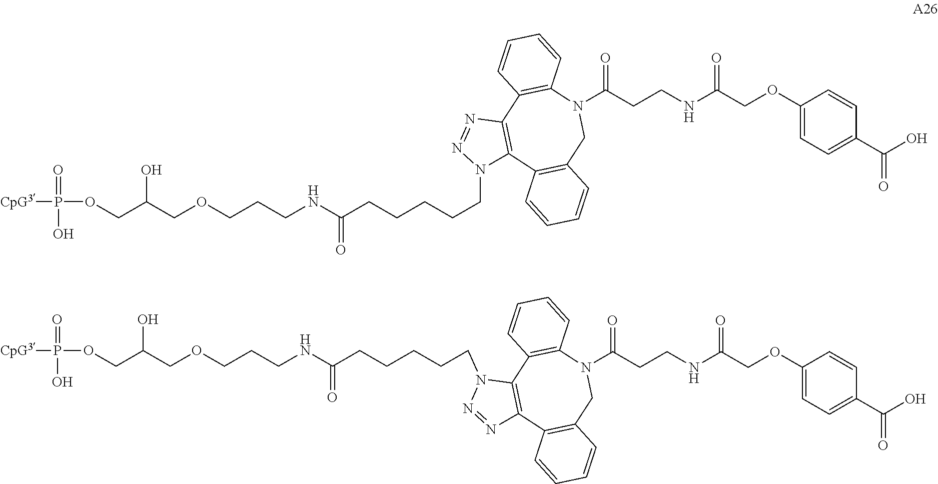

A solution 4.08 mg/mL of I-9 in DMF was prepared. A14 (20 mg, 2.46 mL, 1 eq.) was diluted with PBS, pH 8 (18.4 mL). To the solution, 0.5 M EDTA, pH 8 (888 μL) and 1-9 solution (1.8 mg, 444 μL, 2.0 eq.) were added. The resulting reaction was mixed and then incubated at 22° C. for 1 h. The crude material was buffer exchanged into 10 mM triethylammonium acetate, pH 7.0 via UF/DF using a Vivaspin20 concentrator (PES, 3 kDa MWCO) to give a solution at 3.44 mg/mL (15.5 mg, 78%). The resulting solution was lyophilized to give a mixture of regioisomers A26. Analysis was performed by HPLC (column: ACE Excel Super C18 7.5×2.1 mm, 5 μM; mobile phase: [0.1 M triethylammonium acetate, pH 7.4—ACN]; B %: 0-100%, 10 min). Purity 99%. RT=4.4 min. Calculated mass 10476 Da, measured mass 10476 Da.

### Example 27 Synthesis of A27 (a Mixture of Regioisomers as Shown Below)

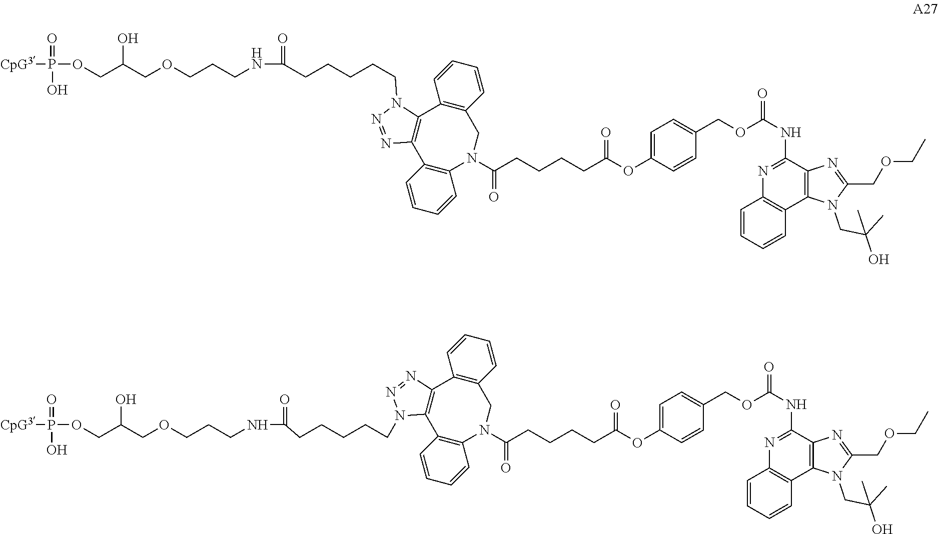

A mixture of regioisomers A27 was synthesized by the reaction of A14 with A22 according to the procedure described in Example 26. Analysis was performed by HPLC (column: Clarity RP C18 100×4.6 mm, 3 μM; mobile phase: [100 mM TEAA—90% ACN/10% 100 mM TEAA]; B %: 10-50%, 30 min). Purity 98%. RT=2.50 min. Calculated mass 10801 Da, measured mass 10802 Da.

### Example 28 Synthesis of A28 (a Mixture of Regioisomers as Shown Below)

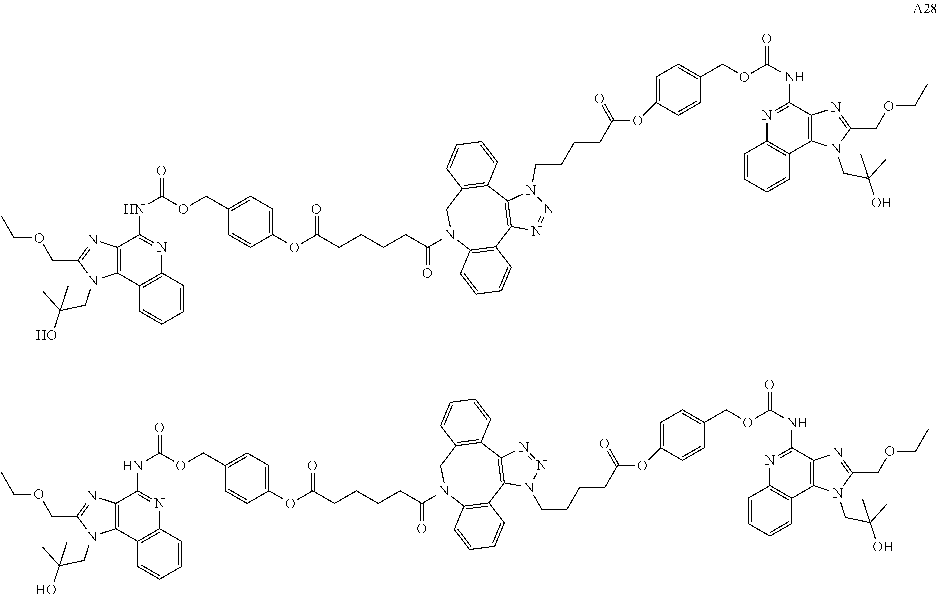

A mixture of regioisomers A28 was synthesized by the reaction of A22 with A20 according to the procedure described in Example 25, and separated by prep-HPLC (column: Phenomenex Gemini-NX C18 75×30 mm, 3 μM; mobile phase: [water (10 mM NH₄HCO₃)-ACN]; B %: 45%-75%, 10 min). Analysis was performed by HPLC (column: X-bridge Shield RP18 50×2.1 mm, 5 μM; mobile phase: [water (10 mM NH₄HCO₃)-ACN]; B %: 10-80%, 4.5 min). Fast-eluting isomer A28-1, purity 98%. RT=3.086 min. Calculated mass 1369 Da, measured mass 1370 Da. Slow-eluting isomer A28-2, purity 100%. RT=3.131 min. Calculated mass 1369 Da, measured mass 1370 Da.

### Example 29 Synthesis of A29

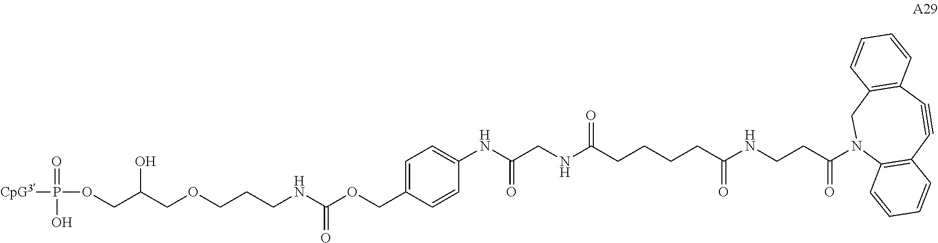

A29 was synthesized by the reaction of A11 with I-14 according to the procedure described in Example 15. Analysis was performed by HPLC (column: X-bridge Shield RP18 50×2.1 mm, 5 μM; mobile phase: [water (10 mM NH₄HCO₃)-ACN]; B %: 0%-60%, 4.5 min). RT=1.51 min Calculated mass 10475 Da, measured mass 10476 Da.

### Example 30. Synthesis of A30 (a Mixture of Regioisomers as Shown Below)

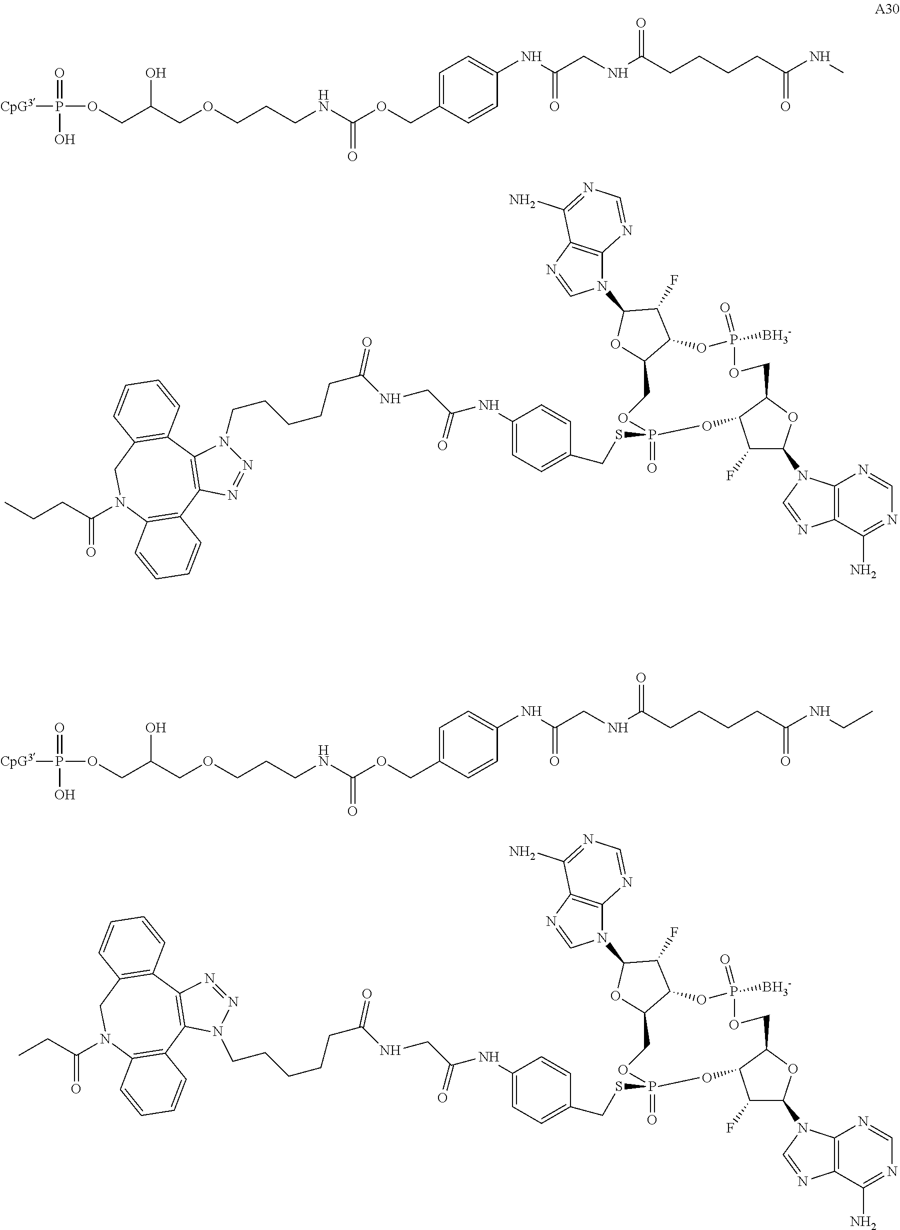

A mixture of regioisomers A30 was synthesized by the reaction of A29 with A8 according to the procedure described in Example 19. Analysis was performed by HPLC (column: Clarity RP C18 100×4.6 mm, 3 μM; mobile phase: [100 mM TEAA— 90% ACN/10% 100 mM TEAA]; B %: 10-50%, 30 min). Purity 94%. Clarity RP C18 3 um 4.6*100 mm, RT=14.16 min. Calculated mass 11453 Da, measured mass 11453 Da.

### Example 31. Synthesis of A31 (a Mixture of Regioisomers as Shown Below)

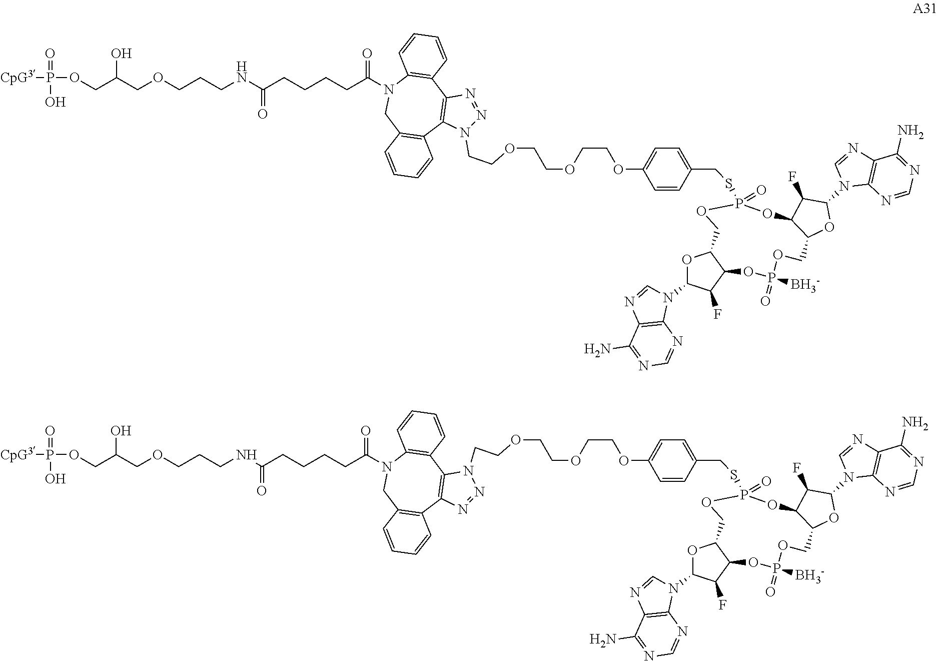

A mixture of regioisomers A31 is synthesized by the reaction of A18 with A9 according to the procedure described in Example 19.

### Example 32. Synthesis of A32 (a Mixture of Regioisomers as Shown Below)

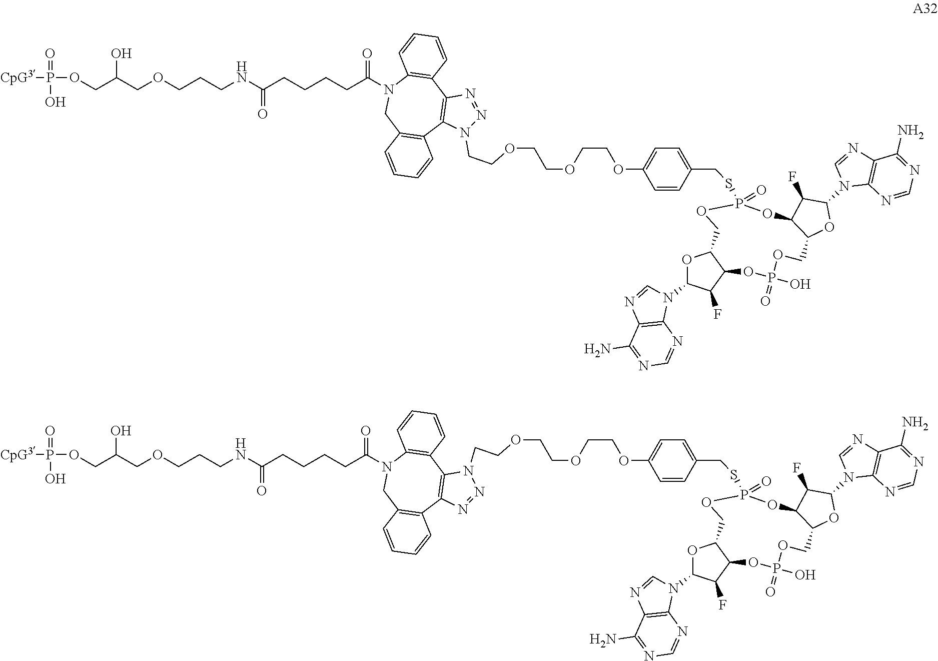

A mixture of regioisomers A32 is synthesized by the reaction of A18 with A10 according to the procedure described in Example 19.

### Example 33. Synthesis of A33 (a Mixture of Regioisomers as Shown Below)

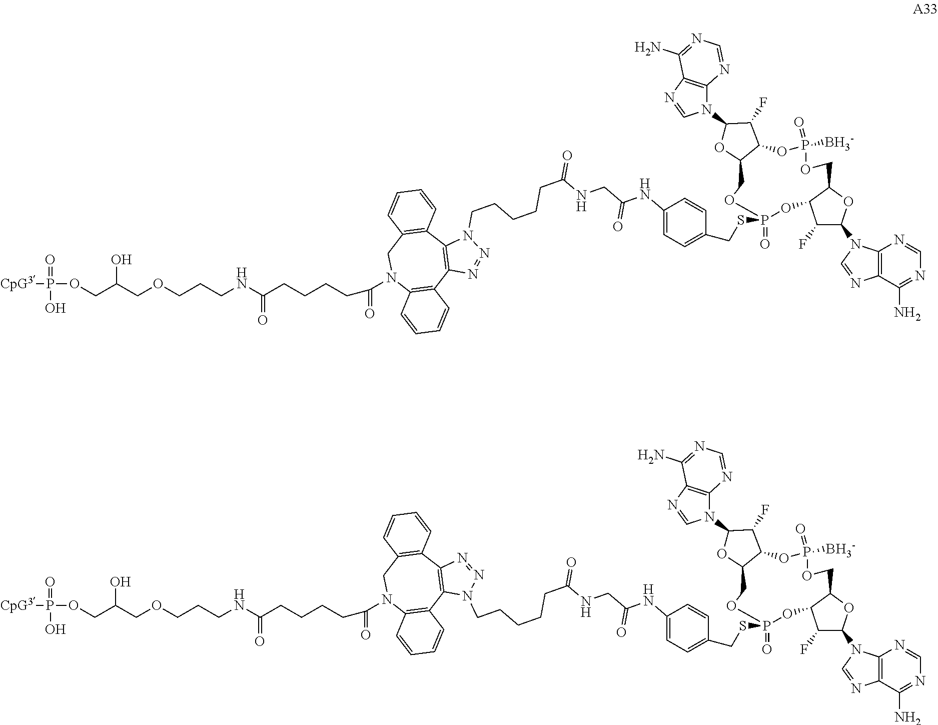

A8 (7.67 mg, 7.84 umol, 2.00 eq) in DMF (0.5 mL) was added to a solution of A18 (40.0 mg, 3.92 umol, 1.00 eq) in PBS (0.5 mL, pH=7.4) at 25° C. and stirred at rt for 2 hrs. The reaction solution was purified by prep-HPLC (neutral condition, column: Clarity RP 21.2*150 mm; mobile phase: [0.1M TEAA-ACN]; B %: 5%-35%, 10 min) to give A33 (17.5 mg, 1.57 umol, 39.7% yield) as a solid. Purity 96%. RT=14.13 min. Calculated mass 11175 Da, measured mass 11170 Da.

### Example 34. Synthesis of A34 (a Mixture of Regioisomers as Shown Below)

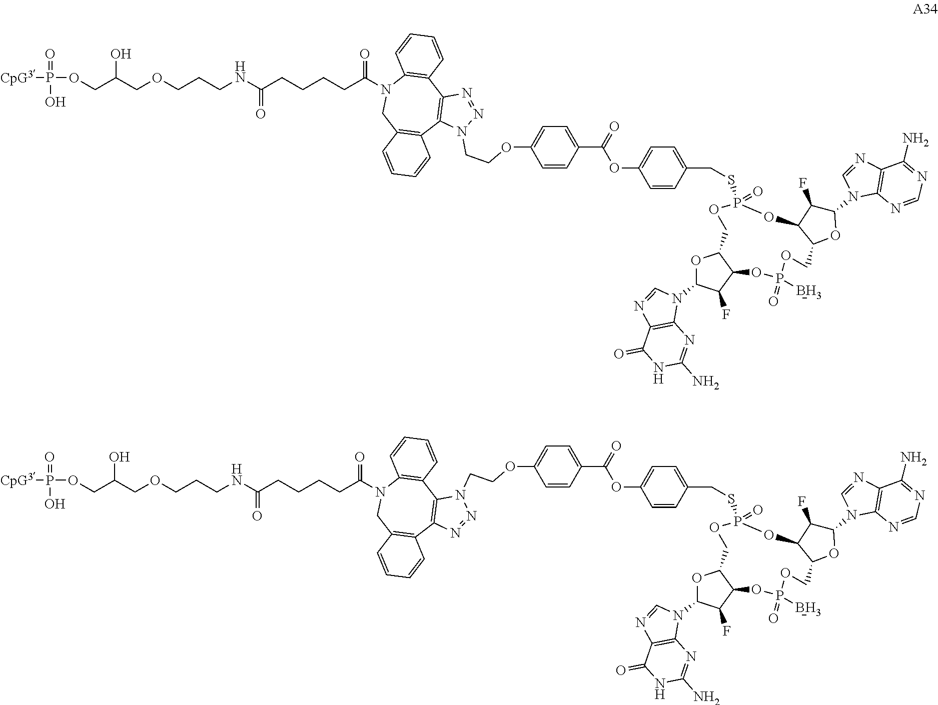

A mixture of regioisomers A34 was synthesized by the reaction of A18 with A3 according to the procedure described in Example 19. Yield: 34.5%, Purity 97%. Clarity RP C18 3 um 4.6*100 mm, RT=13.76 min. Calculated mass 11184 Da, measured mass 11181 Da.

### Example 35. Synthesis of A35 (a Mixture of Regioisomers as Shown Below)

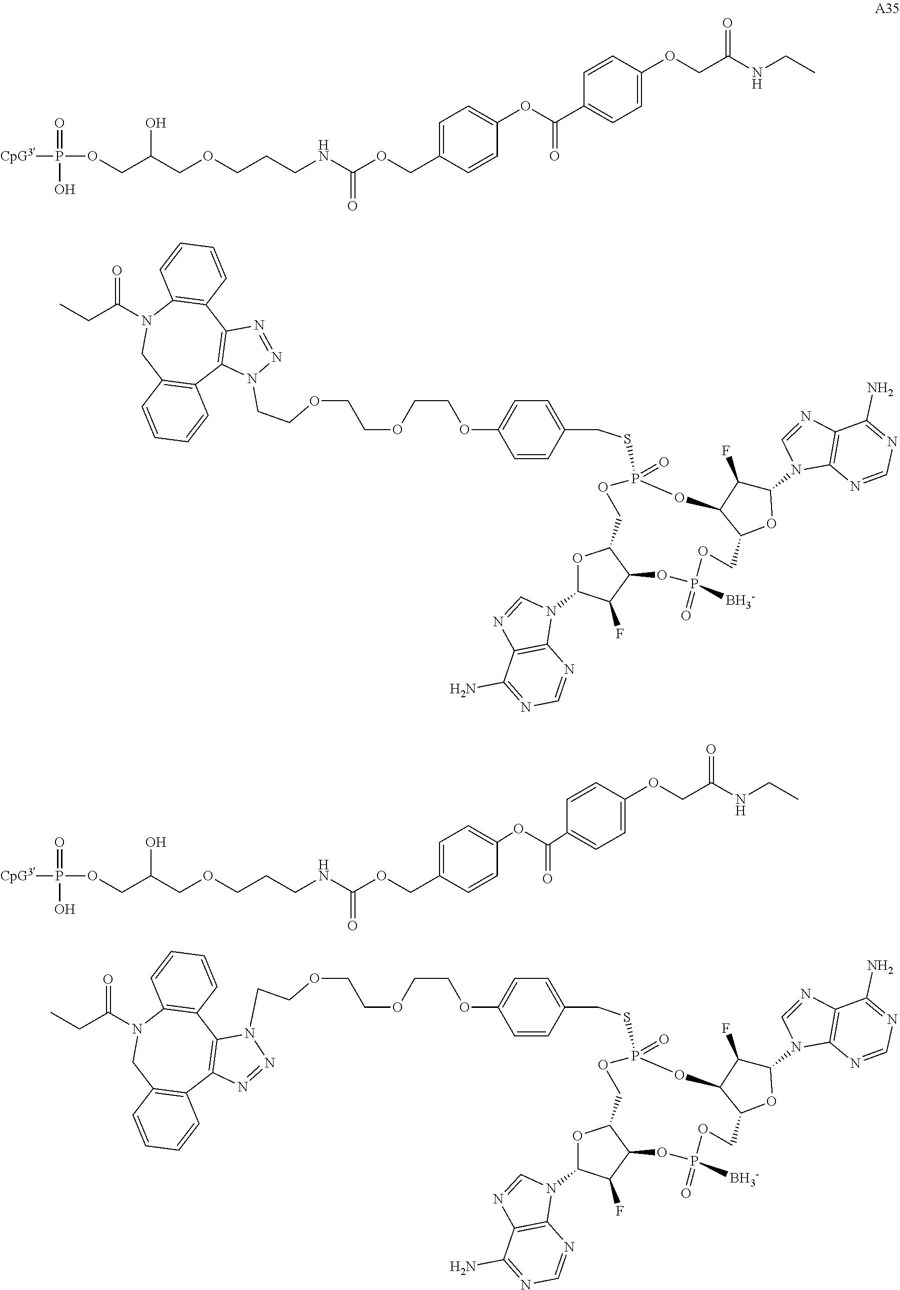

A mixture of regioisomers A35 is synthesized by the reaction of A15 with A9 according to the procedure described in Example 19.

### Example 36. Synthesis of A36 (a Mixture of Regioisomers as Shown Below)

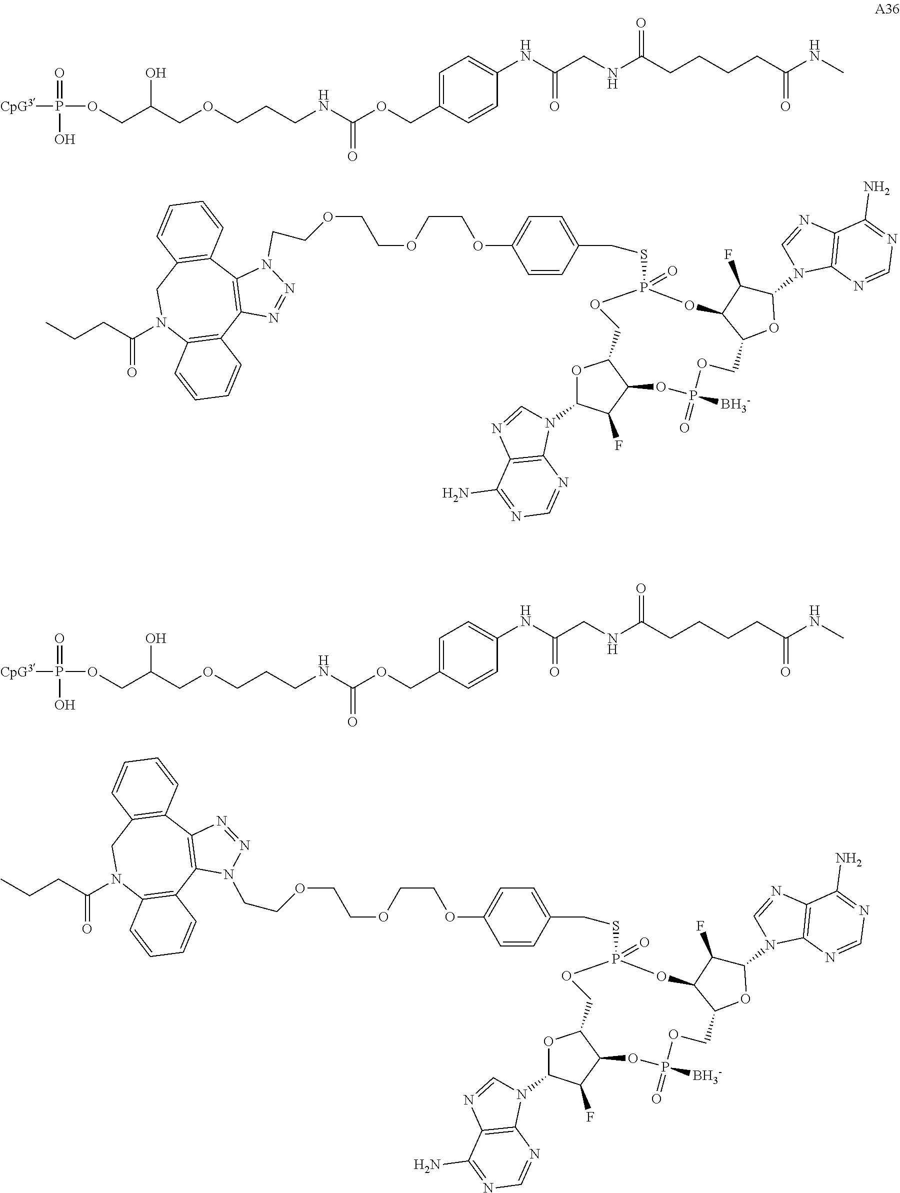

A mixture of regioisomers A36 is synthesized by the reaction of A29 with A9 according to the procedure described in Example 19.

### Example 37. Synthesis of A37 (a Mixture of Regioisomers as Shown Below)

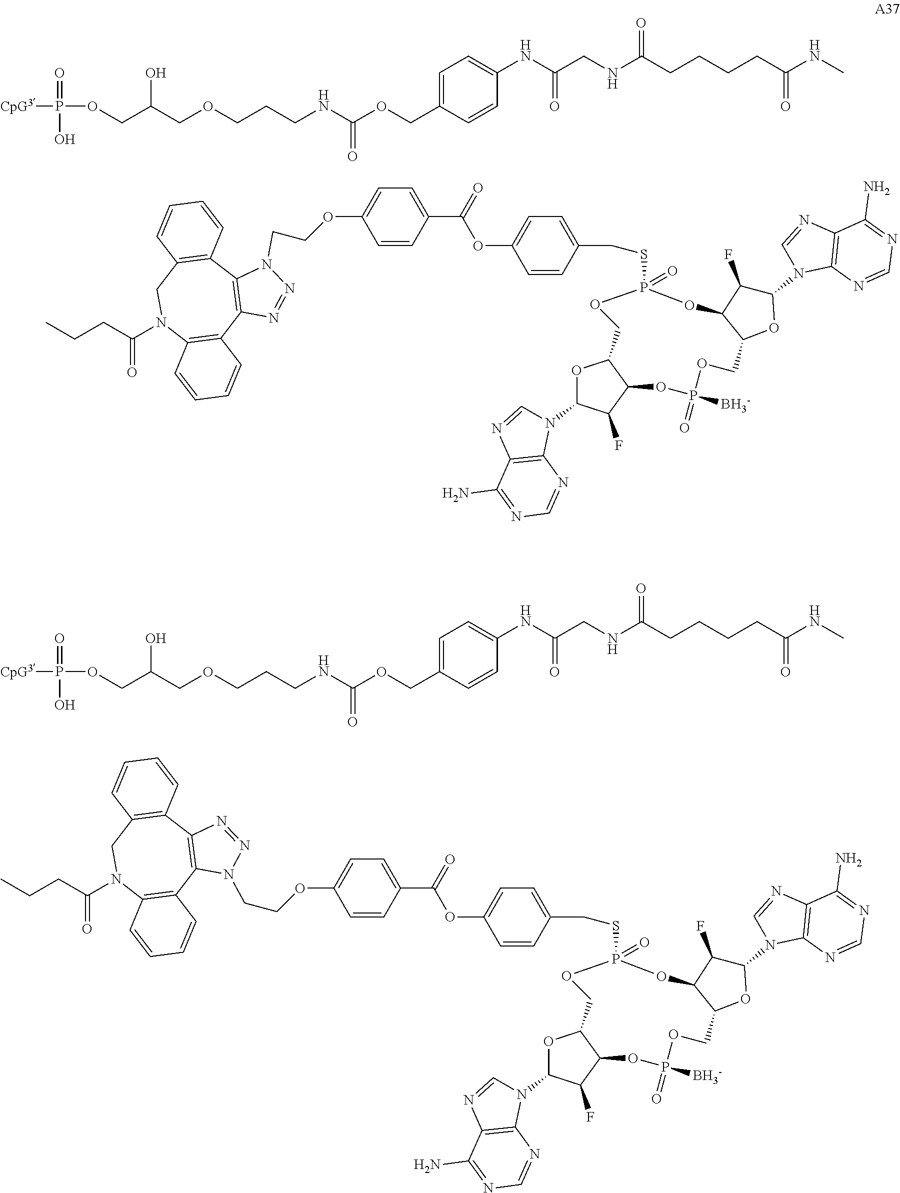

A mixture of regioisomers A37 is synthesized by the reaction of A29 with A7 according to the procedure described in Example 19.

### Example 38. Synthesis of A38 (a Mixture of Regioisomers as Shown Below)

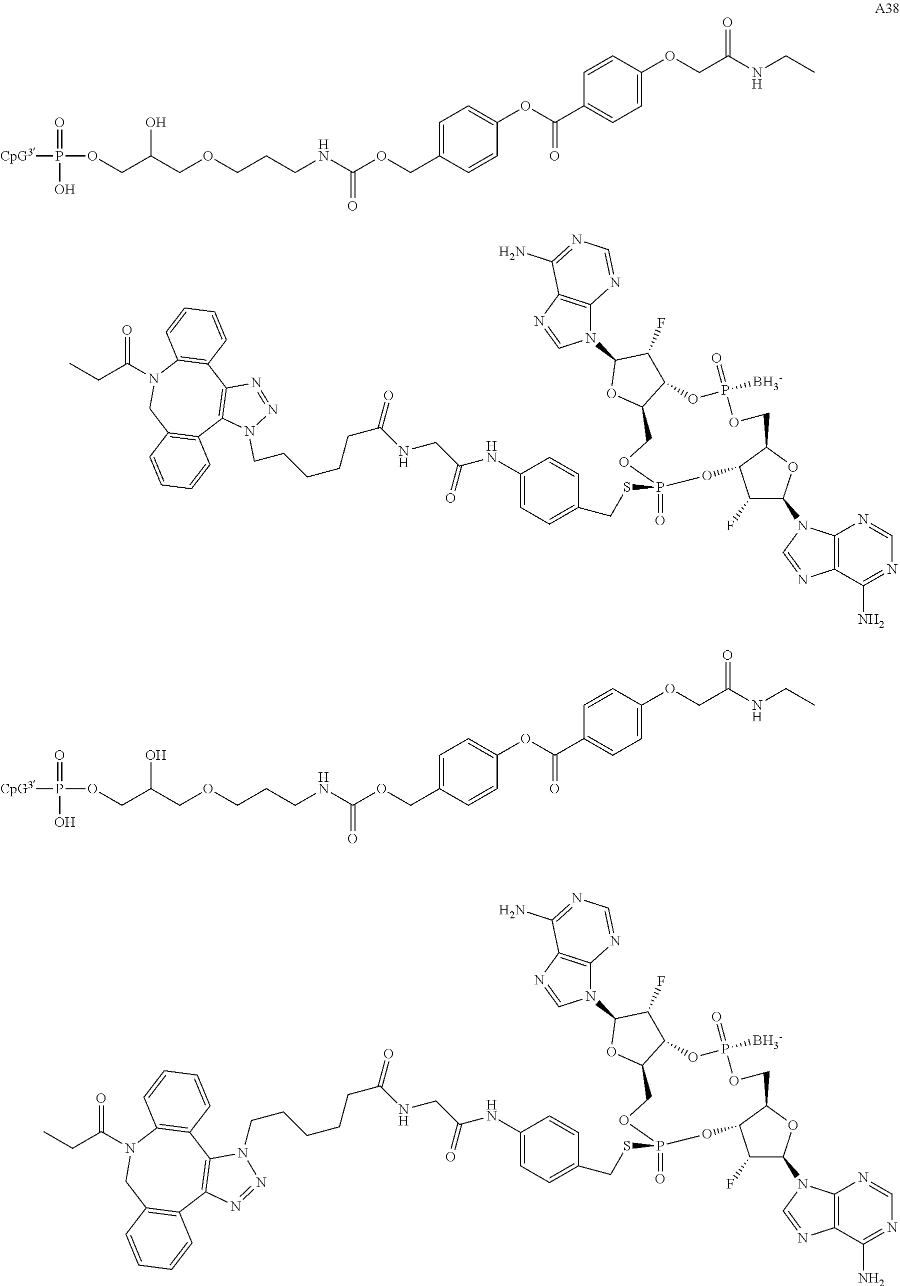

A mixture of regioisomers A38 was synthesized by the reaction of A15 with A8 according to the procedure described in Example A19. Yield: 20%, Purity 97%. Clarity RP C18 3 um 4.6*100 mm, RT=12.95 min. Calculated mass 11446 Da, measured mass 11450 Da.

### Example 39. Synthesis of A39 (a Mixture of Regioisomers as Shown Below)

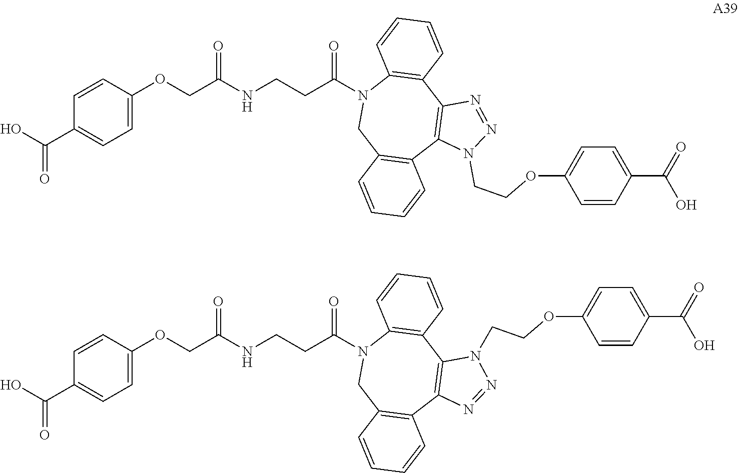 [SMILES: O=C(COc1ccc(C(=O)O)cc1)NCCC(=O)N1Cc2ccccc2-c2c(nnn2CCOc2ccc(C(=O)O)cc2)-c2ccccc21 ; O=C(COc1ccc(C(=O)O)cc1)NCCC(=O)N1Cc2ccccc2-c2nnn(CCOc3ccc(C(=O)O)cc3)c2-c2ccccc21]

To a solution of A17 (20 mg, 1.74 umol) in THF (0.5 mL) is added 2.5 M aq. NaOH (3.5 umol). The mixture is warmed to 60° C., stirred for 3-8 h, and cooled to rt. After addition of 1 N aq. HCl, the mixture is extracted with CH₂C12 twice. The combined organic layer is dried over MgSO₄, filtered, and concentrated. The obtained residue is purified to afford a mixture of regioisomers A39.

### Example 40. Synthesis of A40 (a Mixture of Regioisomers as Shown Below)

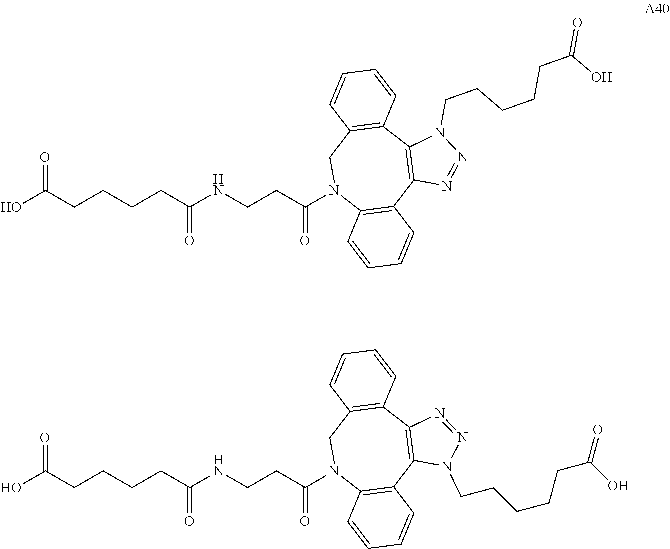 [SMILES: O=C(O)CCCCCn1nnc2c1-c1ccccc1CN(C(=O)CCNC(=O)CCCCC(=O)O)c1ccccc1-2 ; O=C(O)CCCCCn1nnc2c1-c1ccccc1N(C(=O)CCNC(=O)CCCCC(=O)O)Cc1ccccc1-2]

1N HCl (0.5 ml) is added to a solution of A30 (10 mg, 0.87 umol) in 2 ml of water, the mixture is stirred at rt overnight and then extracted with CH₂C12 twice. The combined organic layer is dried over MgSO₄, filtered, and concentrated. The obtained residue is purified to afford a mixture of regioisomers A40.

### Example 41. Synthesis of A41 (a Mixture of Regioisomers as Shown Below)

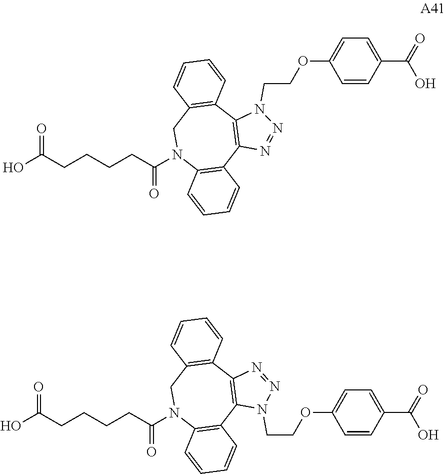 [SMILES: O=C(O)CCCCC(=O)N1Cc2ccccc2-c2c(nnn2CCOc2ccc(C(=O)O)cc2)-c2ccccc21 ; O=C(O)CCCCC(=O)N1Cc2ccccc2-c2nnn(CCOc3ccc(C(=O)O)cc3)c2-c2ccccc21]

A mixture of regioisomers A41 is synthesized from A25 according to the procedure described in Example 39.

### Example 42. Synthesis of A42 (a Mixture of Regioisomers as Shown Below)

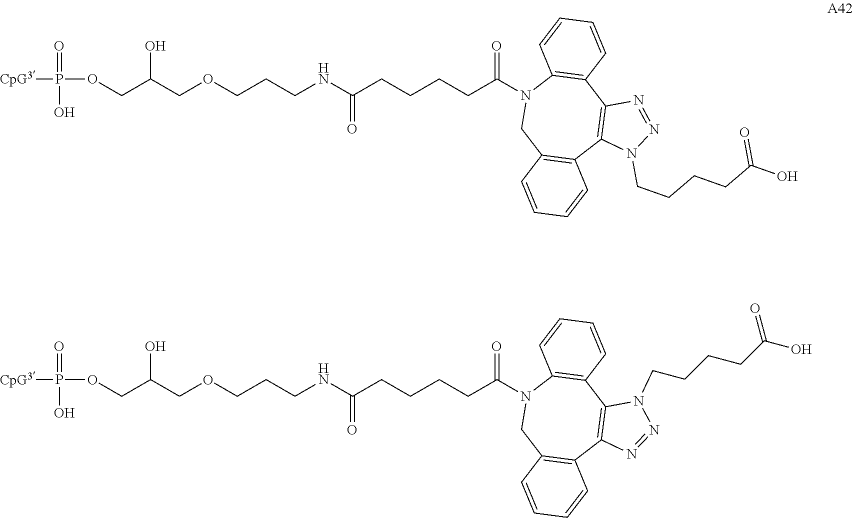

A mixture of regioisomers A42 is synthesized by the reaction of A18 and 5-azidopentanoic acid according to the procedure described in Example 19.

### Example 43. Synthesis of A43-I and A43-II

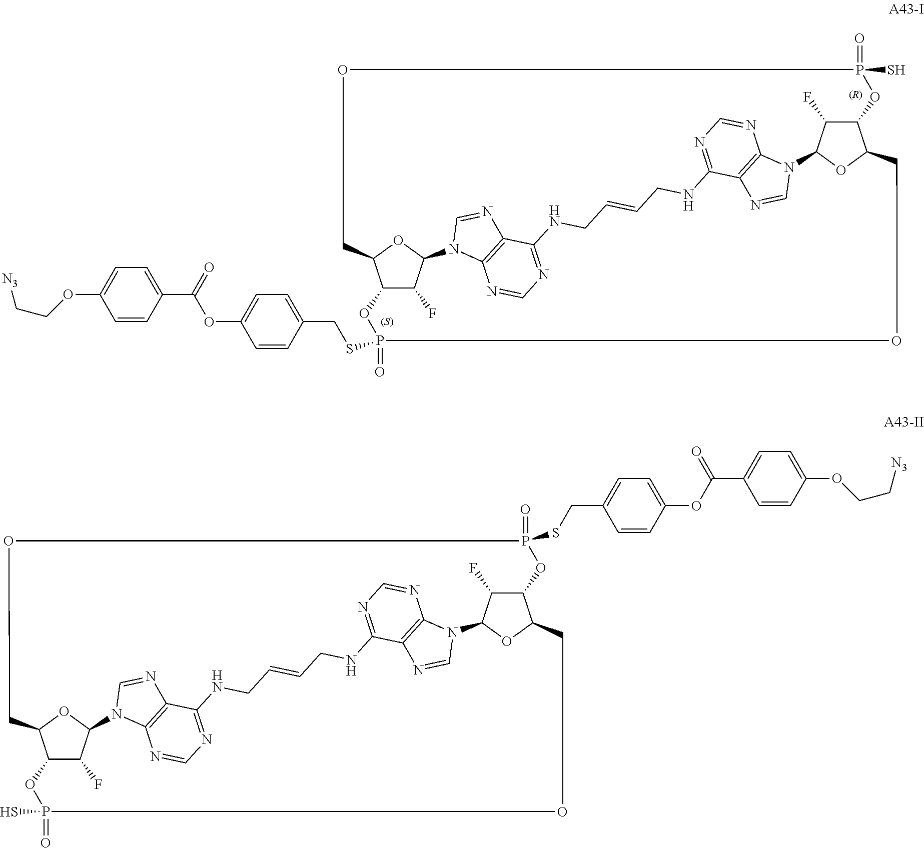

To a solution of ammonium salt of (19S,22R,23R,23aR,25R,27aR,29R,210R,210aR,212S,214aR,39S,E)-23,210-difluoro-25,212-dimercapto-23,23a,27a,29,210,210a,214,214a-octahydro-19H,22H,27H,39H-4,9-diaza-1,3(9,6)-dipurina-2(2,9)-difuro [3,2-d: 3′,2′-j][1,3,7,9]tetraoxa[2,8]diphosphacyclododecinacyclononaphan-6-ene 25,212-dioxide (can be synthesized according to the synthetic method described in WO 2019232392) (9.75 mg, 0.013 mmol, 1.00 eq) in DMF (0.25 mL), is added 4-(iodomethyl)phenyl 4-(2-azidoethoxy)benzoate (6.35 mg, 0.015 mmol, 1.20 eq) in a mixed solvent of THF (0.25 mL) and acetone (0.25 mL). The resulting solution is stirred overnight at room temperature. The crude product is purified by Prep-HPLC to give A43-I and A43-II.

### Example 44. Synthesis of A44 (a Mixture of Regioisomers as Shown Below)

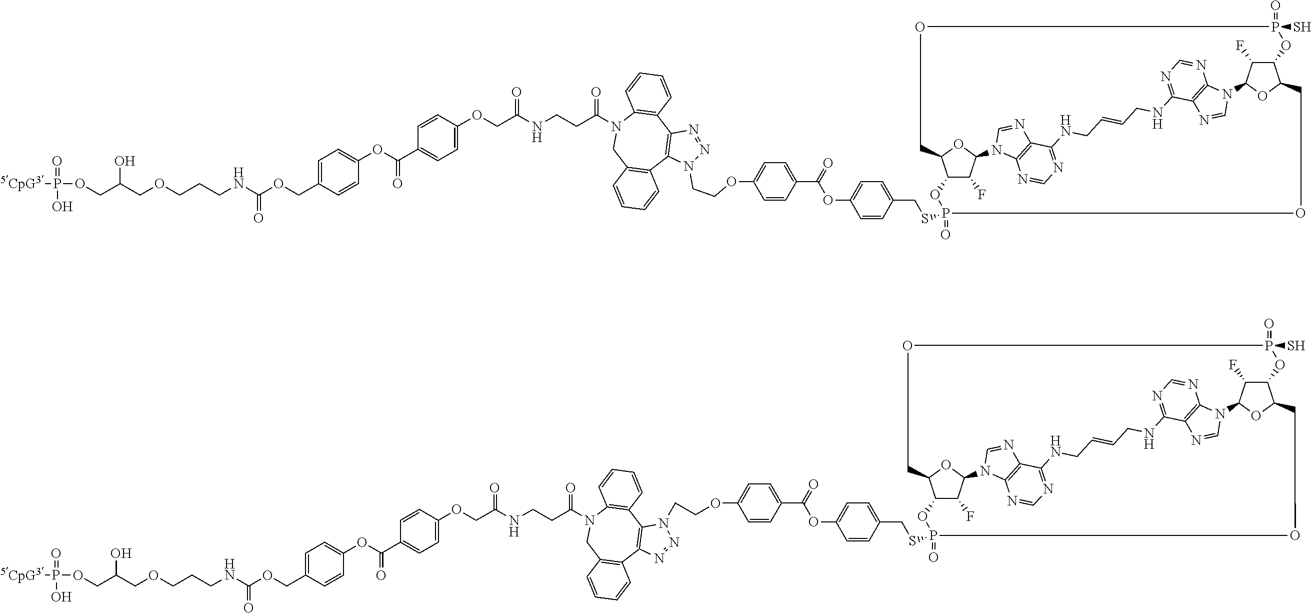

A mixture of regioisomers A44 is synthesized by the reaction of A15 with A43-I according to the procedure described in Example 16.

### Example 45. Synthesis of A45 (a Mixture of Regioisomers as Shown Below)

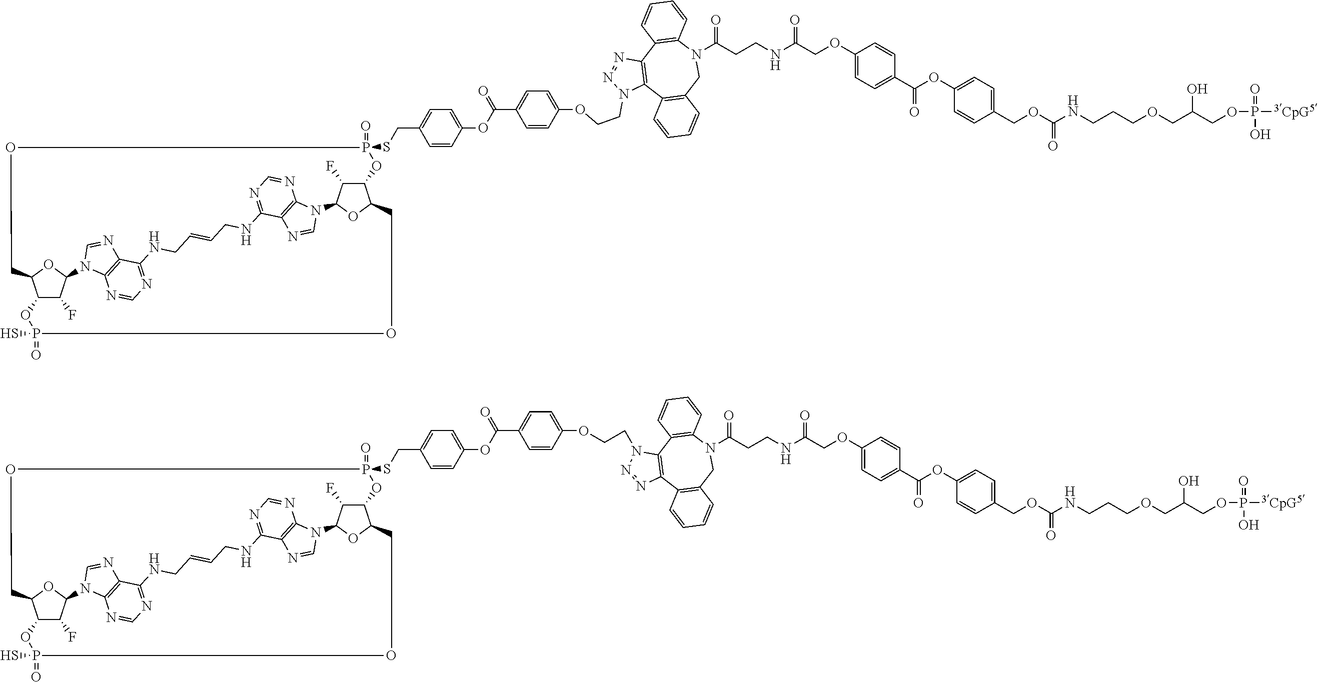

A mixture of regioisomers A45 is synthesized by the reaction of A15 with A43-II according to the procedure described in Example 16.

### Example 46. Synthesis of A46

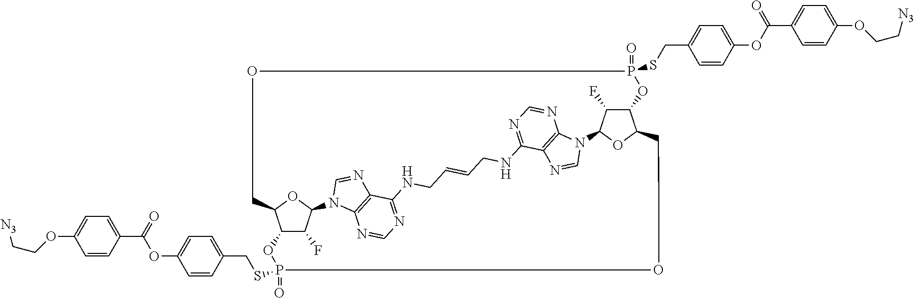 [SMILES: [N-]=[N+]=NCCOc1ccc(C(=O)Oc2ccc(CS[P@@]3(=O)OC[C@H]4O[C@@H]5[C@H](F)[C@@H]4O[P@](=O)(SCc4ccc(OC(=O)c6ccc(OCCN=[N+]=[N-])cc6)cc4)OC[C@H]4O[C@H]([C@H](F)[C@@H]4O3)n3cnc4c(ncnc43)NC/C=C/CNc3ncnc4c3ncn45)cc2)cc1]

To a solution of ammonium salt of (19S,22R,23R,23aR,25R,27aR,29R,210R,210aR,212S,214aR,39S,E)-23,210-difluoro-25,212-dimercapto-23,23a,27a,29,210,210a,214,214a-octahydro-19H,22H,27H,39H-4,9-diaza-1,3(9,6)-dipurina-2(2,9)-difuro [3,2-d:3′,2′-j][1,3,7,9]tetraoxa[2,8]diphosphacyclododecinacyclononaphan-6-ene 25,212-dioxide (can be synthesized according to the synthetic method described in WO 2019232392) (10.00 mg, 0.013 mmol, 1.00 eq) in DMF (0.25 mL), is added 4-(iodomethyl)phenyl 4-(2-azidoethoxy)benzoate (13.75 mg, 0.033 mmol, 2.50 eq) in a mixed solvent of THF (0.5 mL) and acetone (0.5 mL). The resulting solution is stirred overnight at room temperature. The crude product is purified by Prep-HPLC to give A46.

### Example 47. Synthesis of A47 (a Mixture of Regioisomers as Shown Below)

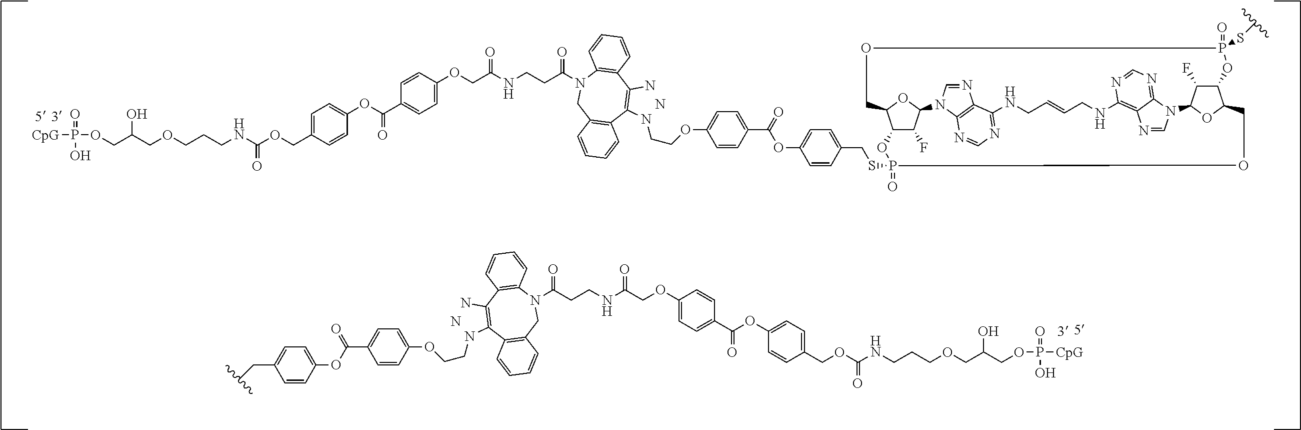

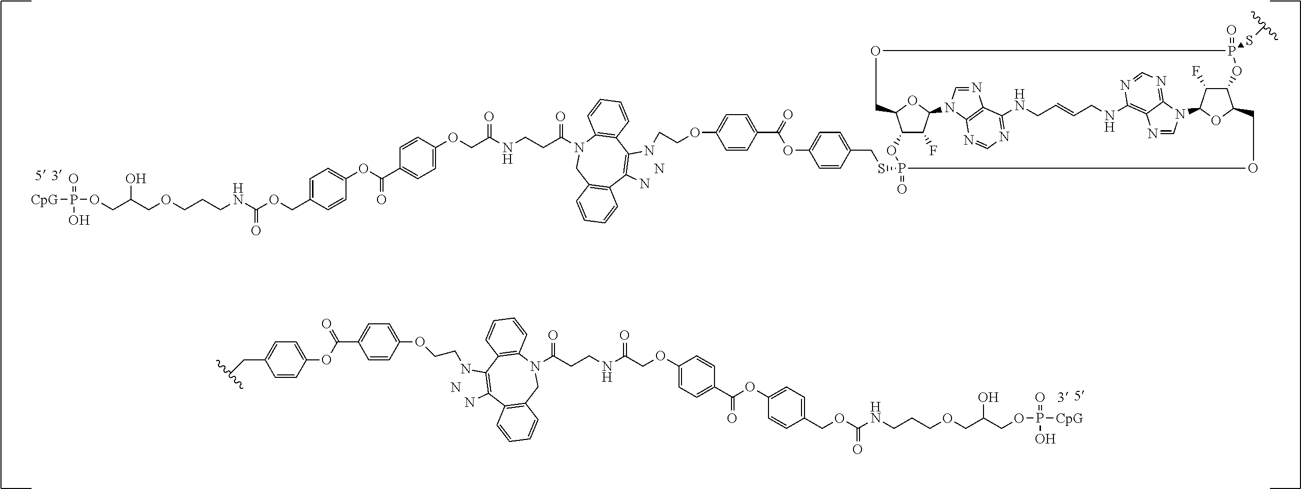

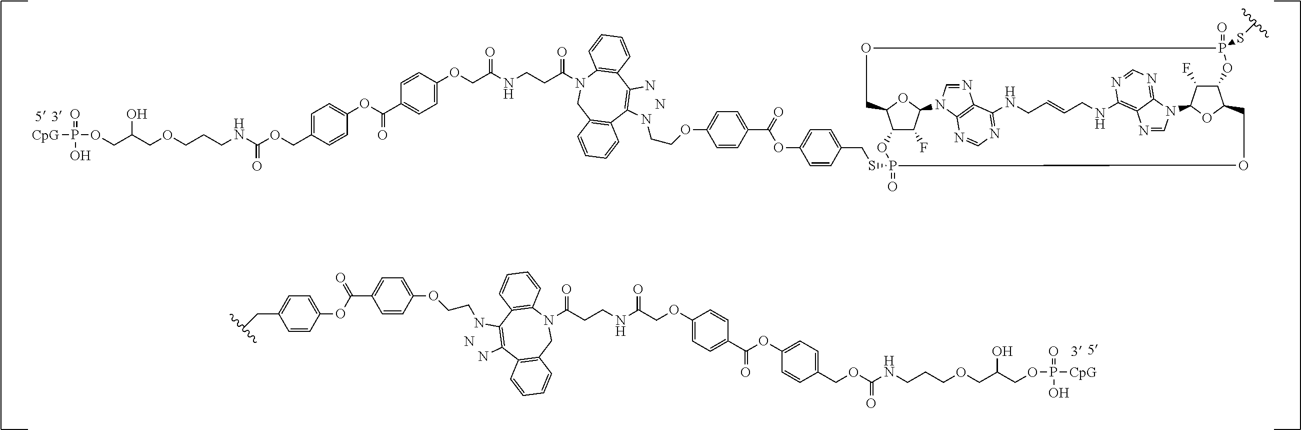

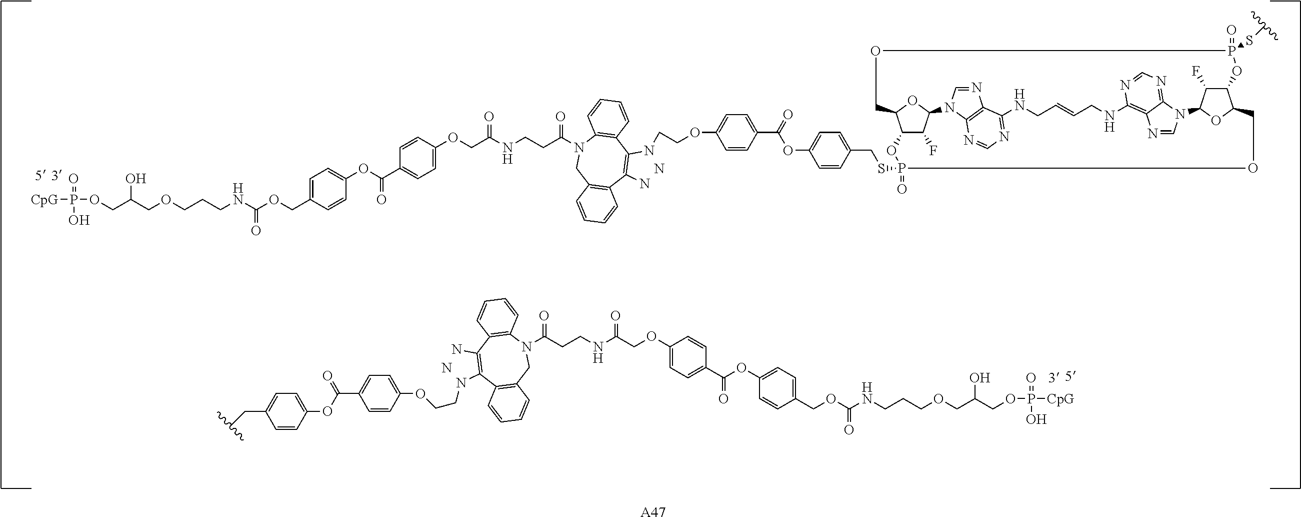

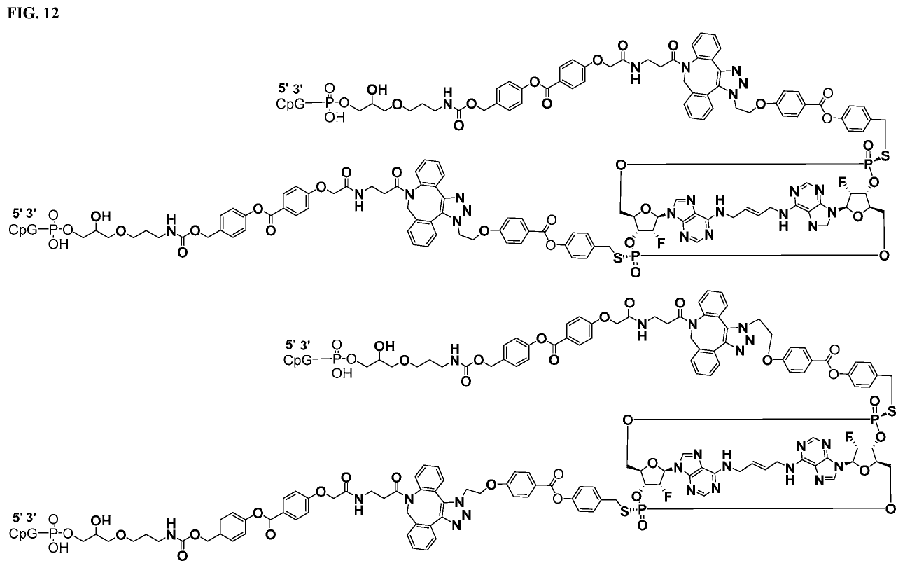

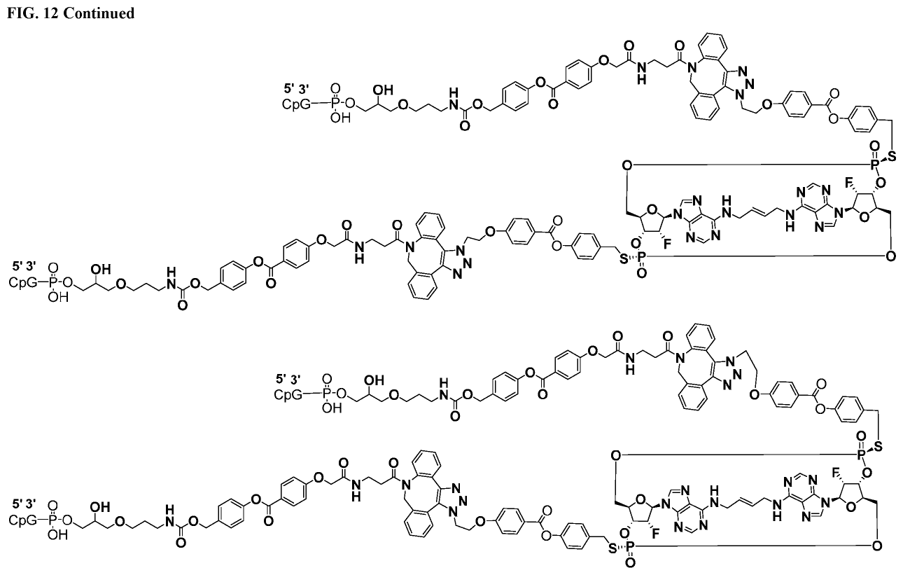

To a solution of A15 (47 mg, 2 eq.) in PBS, pH 7.4 (8.27 mL), is added A46 (3.0 mg, 1 eq.) in a 1:1 solution of PBS, pH 7.4/DMSO (1.77 mL). The resulting reaction is mixed and incubated at 22° C. for 2 h. The crude reaction is buffer exchanged into 20 mM ammonium bicarbonate, pH 7.0 via UF/DF using a Vivaspin20 concentrator (PES, 3 kDa MWCO). The material is subsequently purified by prep-HPLC, fractions containing product are combined and the volatiles are evaporated in vacuo. The material is then buffer exchanged into 20 mM ammonium bicarbonate, pH 7.0 via UF/DF using a Vivaspin20 concentrator (PES, 3 kDa MWCO). The resulting solution is lyophilized to give a mixture of regioisomers A47 (see FIG. 12).

### Example 48. Synthesis of A48 (a Mixture of Regioisomers as Shown Below)

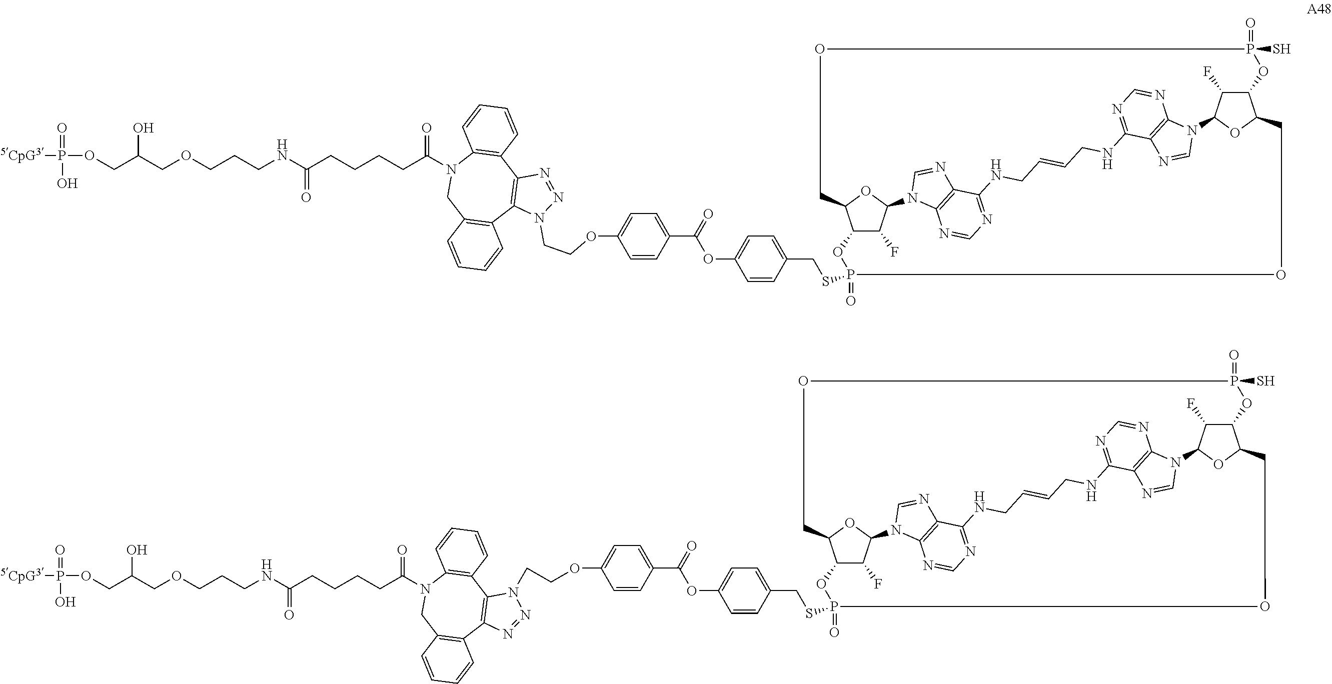

A mixture of regioisomers A48 is synthesized by the reaction of A18 with A43-I according to the procedure described in Example 19.

### Example 49. Synthesis of A49 (a Mixture of Regioisomers as Shown Below)

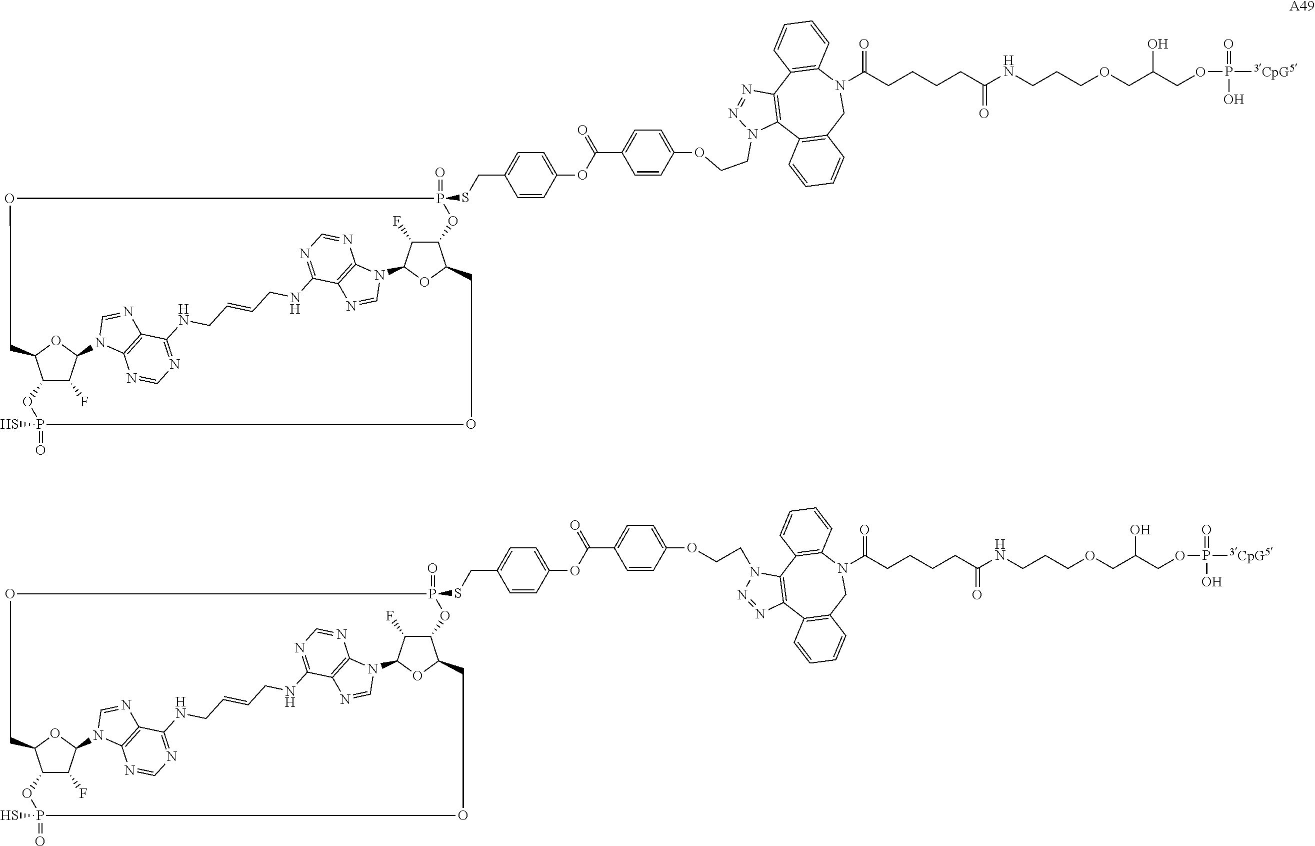

A mixture of regioisomers A49 is synthesized by the reaction of A18 with A43-II according to the procedure described in Example 19.

### Example 50. Synthesis of A50 (a Mixture of Regioisomers as Shown Below)

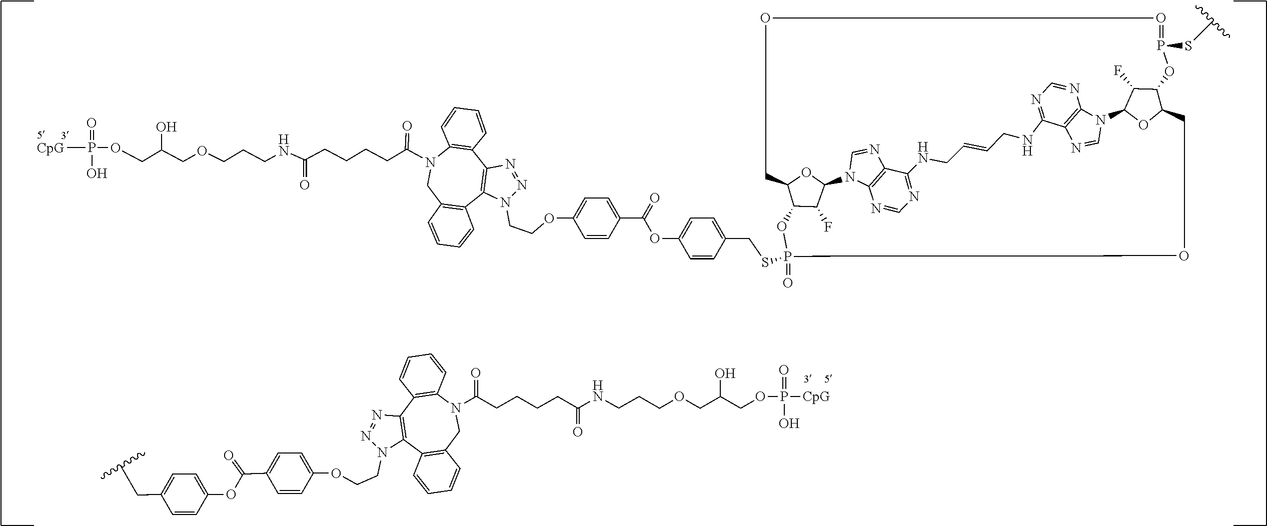

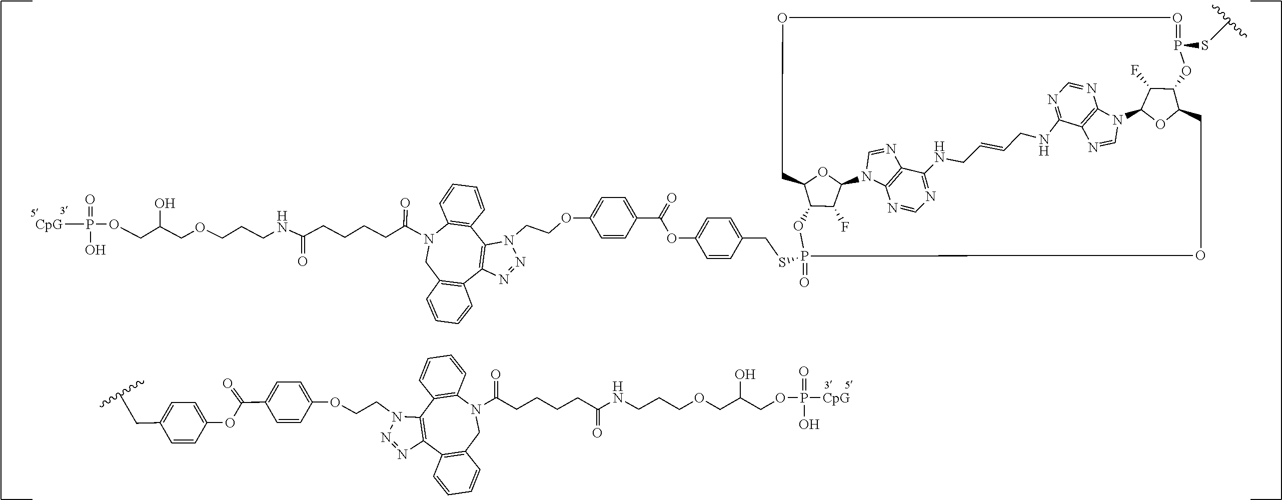

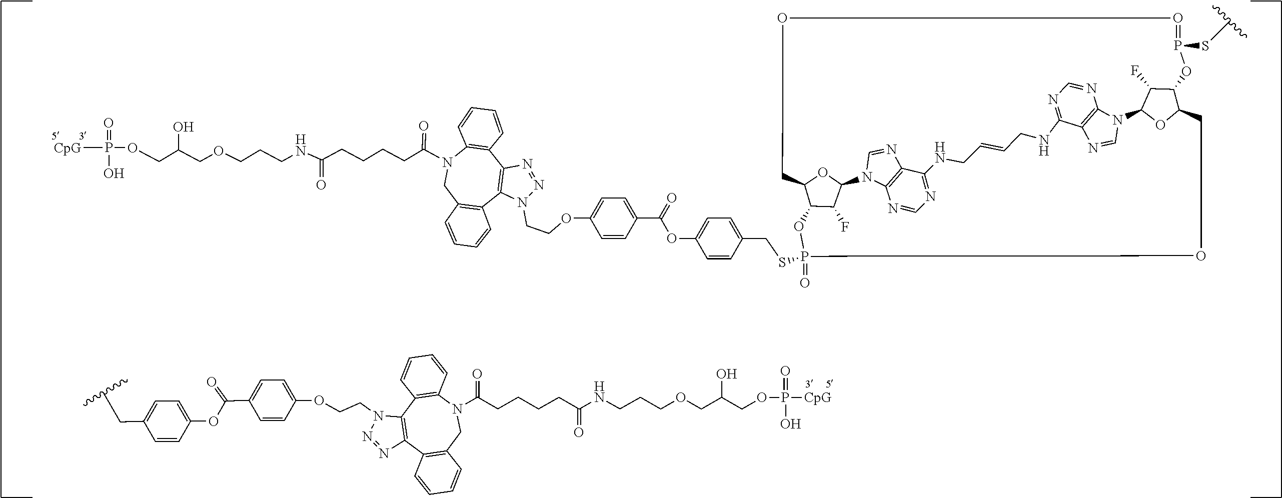

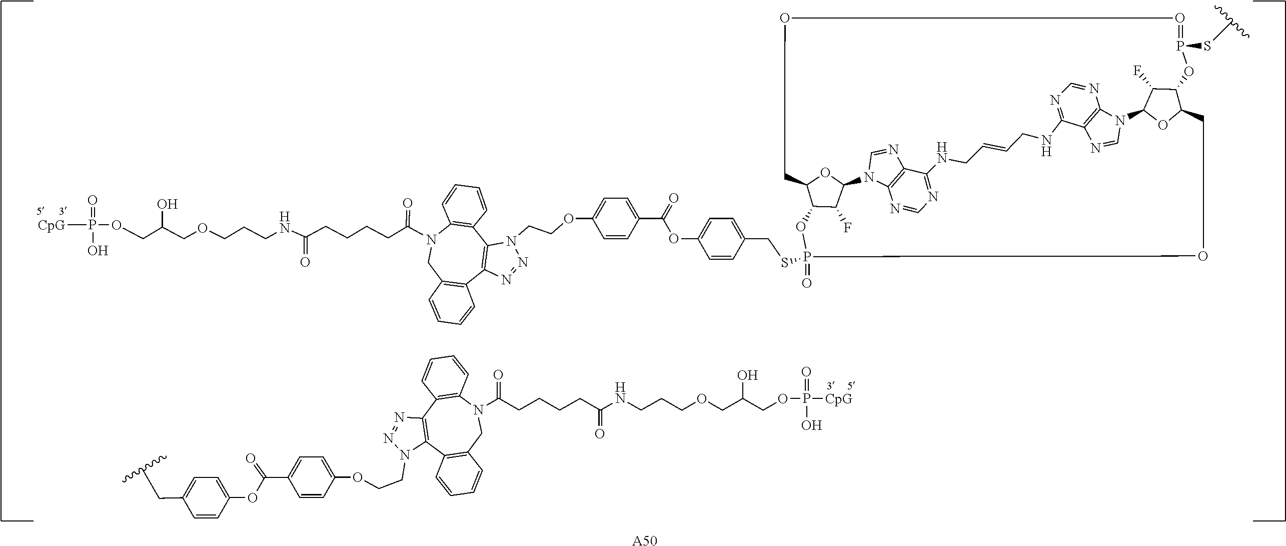

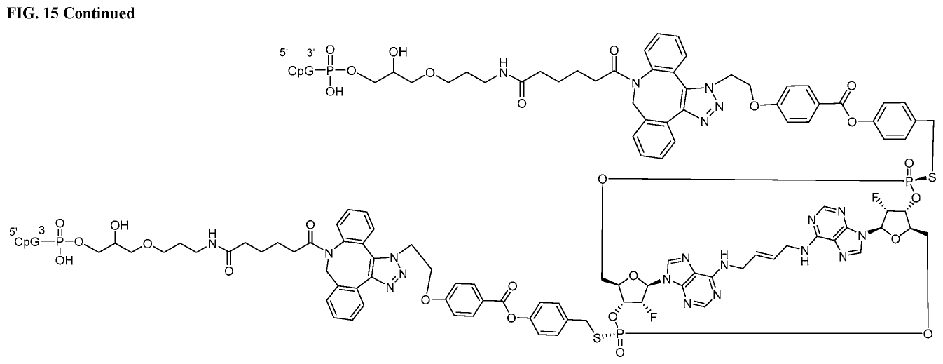

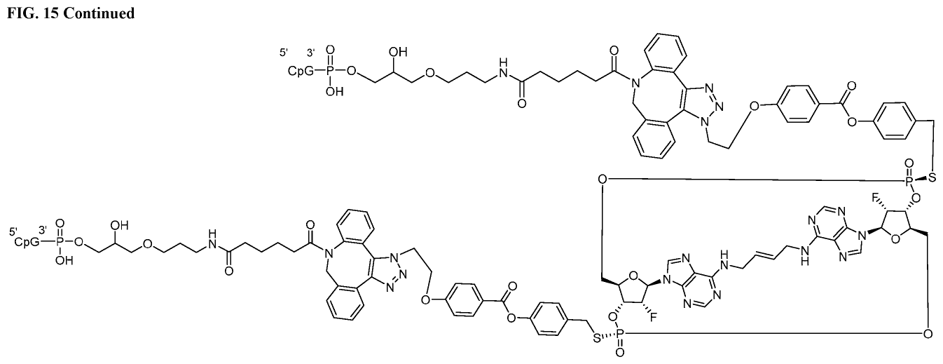

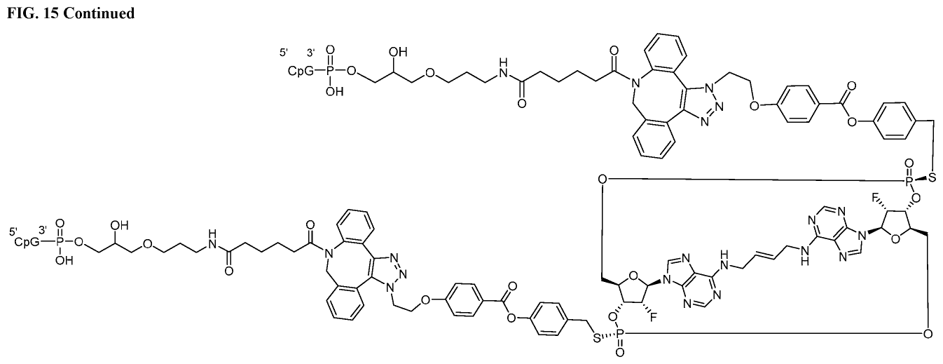

A18 (46.0 mg, 4.48 μmol, 2.00 eq) is dissolved in PBS, pH 7.4 (0.6 mL). To this solution is added A46 (3.0 mg, 2.24 mol, 1.00 eq) dissolved in a 1:1 solution of PBS, pH 7.4/DMSO (0.6 mL). The resulting reaction is mixed and incubated at 20° C. for 16 h. The crude mixture is purified by prep-HPLC. The fractions containing the product are combined and lyophilized to give a mixture of regioisomers A50 (see FIG. 15).

### Example 51. Synthesis of A51-I and A51-II

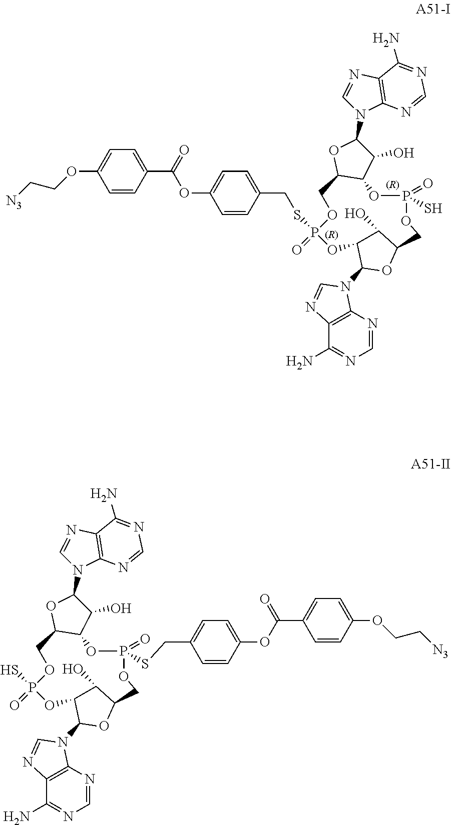

A51-I and A51-II are synthesized by the reaction of ammonium salt of ADU-S100 with 4-(iodomethyl)phenyl 4-(2-azidoethoxy)benzoate according to the procedure described in Example 43.

### Example 52. Synthesis of A52

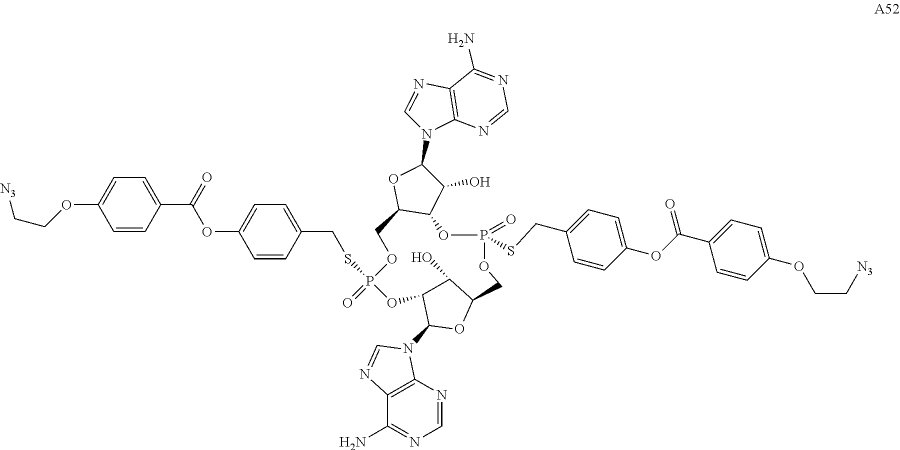 [SMILES: [N-]=[N+]=NCCOc1ccc(C(=O)Oc2ccc(CS[P@]3(=O)OC[C@H]4O[C@@H](n5cnc6c(N)ncnc65)[C@H](O)[C@@H]4O[P@](=O)(SCc4ccc(OC(=O)c5ccc(OCCN=[N+]=[N-])cc5)cc4)OC[C@H]4O[C@@H](n5cnc6c(N)ncnc65)[C@H](O3)[C@@H]4O)cc2)cc1]

A52 is synthesized by the reaction of ammonium salt of ADU-S100 with 4-(iodomethyl)phenyl 4-(2-azidoethoxy)benzoate according to the procedure described in Example 46.

### Example 53. Synthesis of A53 (a Mixture of Regioisomers as Shown Below)

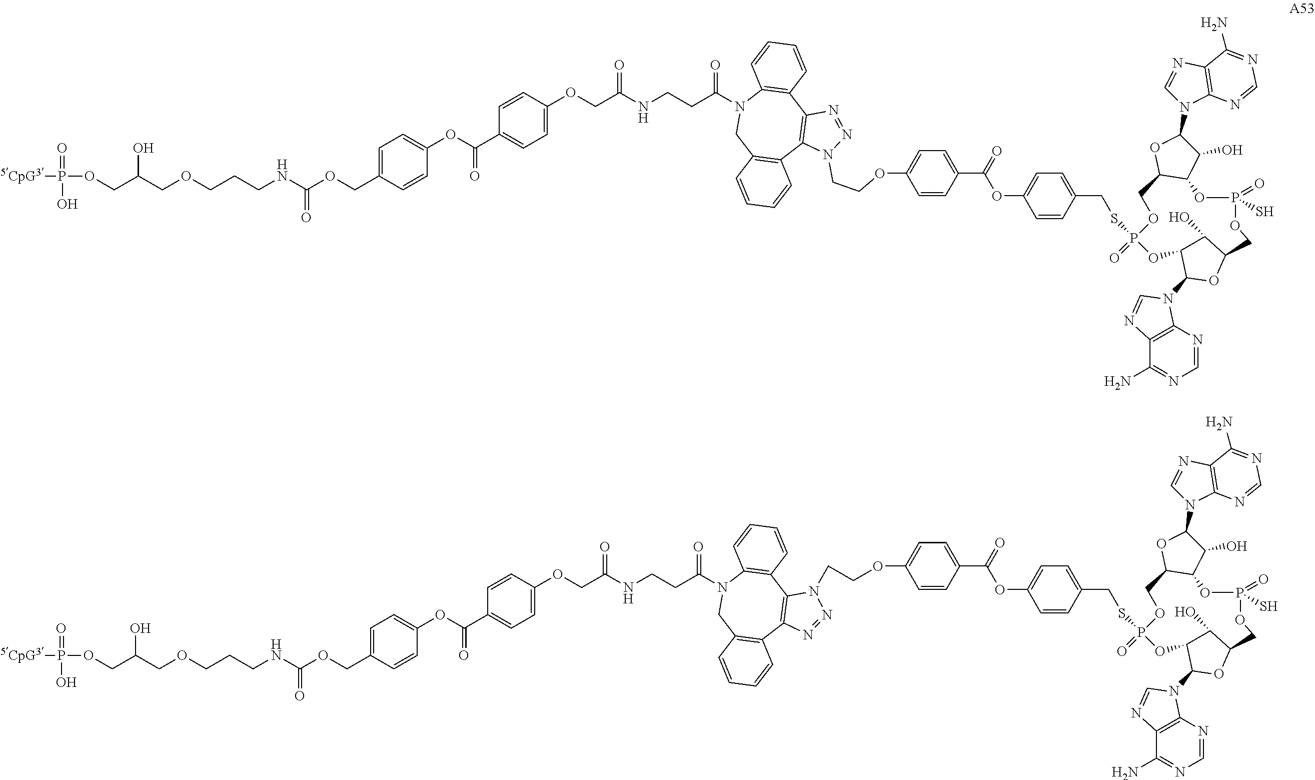

A mixture of regioisomers A53 is synthesized by the reaction of A15 with A51-I according to the procedure described in Example 16.

### Example 54. Synthesis of A54 (a Mixture of Regioisomers as Shown Below)

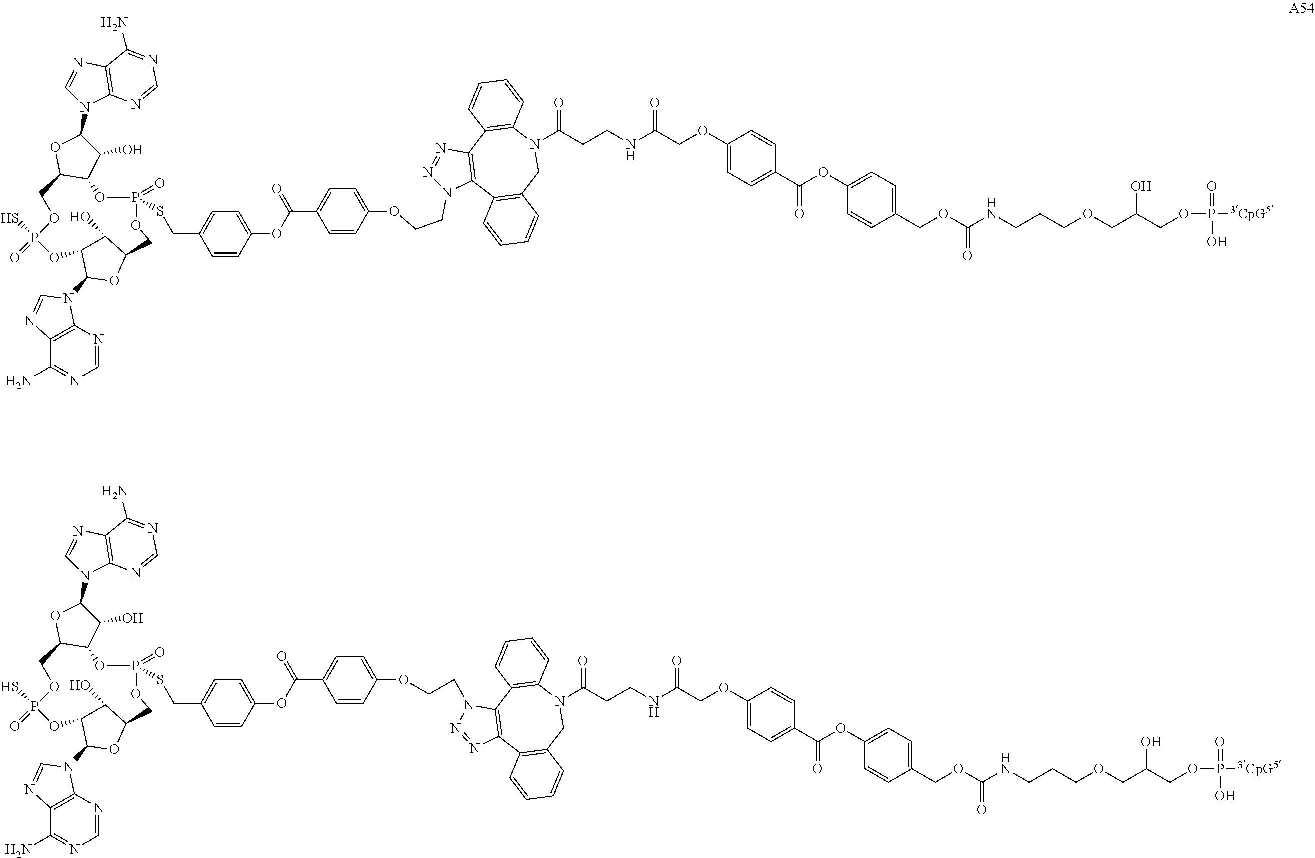

A mixture of regioisomers A54 is synthesized by the reaction of A15 with A51-II according to the procedure described in Example 16.

### Example 55. Synthesis of A55 (a Mixture of Regioisomers as Shown Below)

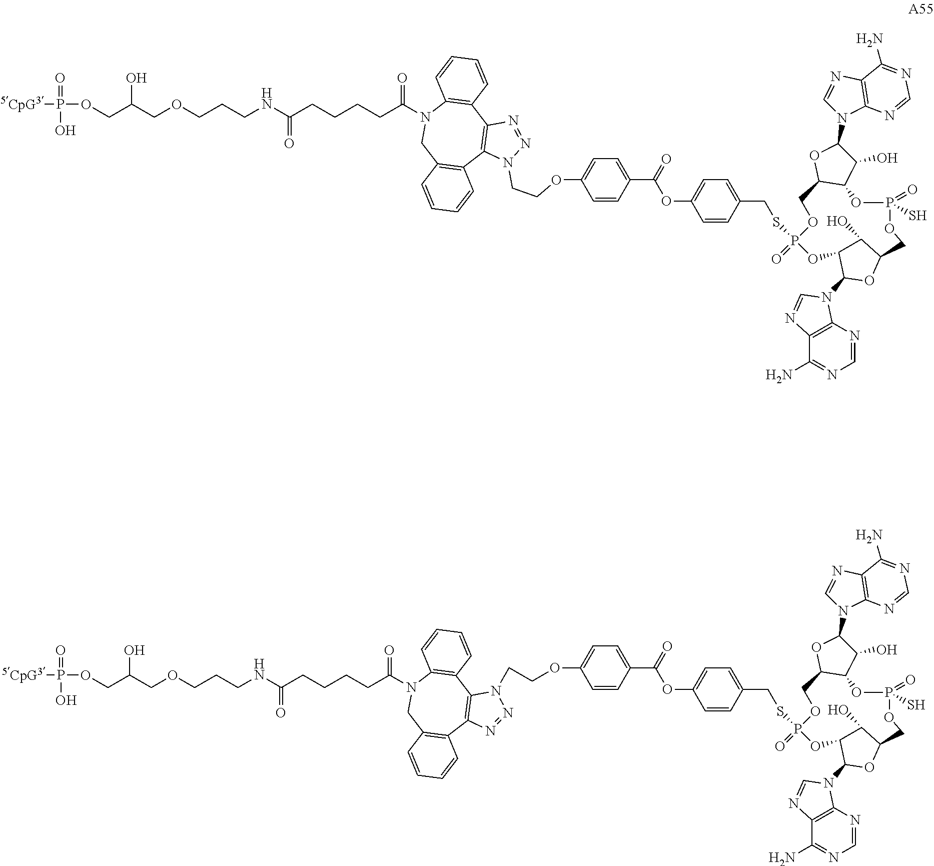

A mixture of regioisomers A55 is synthesized by the reaction of A18 with A51-I according to the procedure described in Example 19.

### Example 56. Synthesis of A56 (a Mixture of Regioisomers as Shown Below)

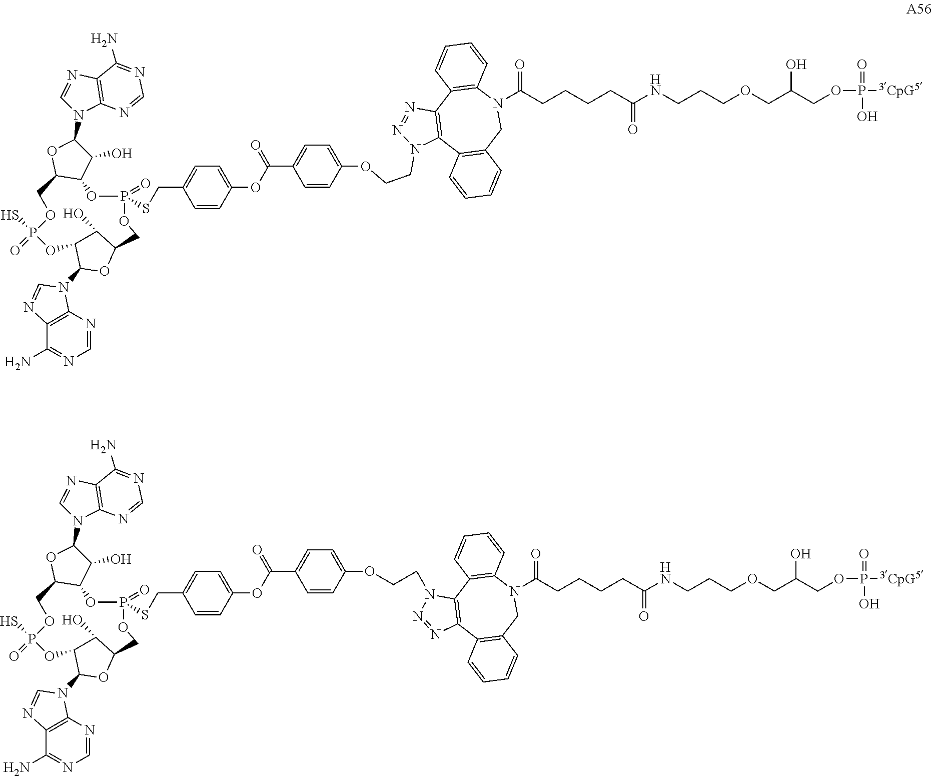

A mixture of regioisomers A56 is synthesized by the reaction of A18 with A51-II according to the procedure described in Example 19.

### Example 57. Synthesis of A57 (a Mixture of Regioisomers as Shown Below)

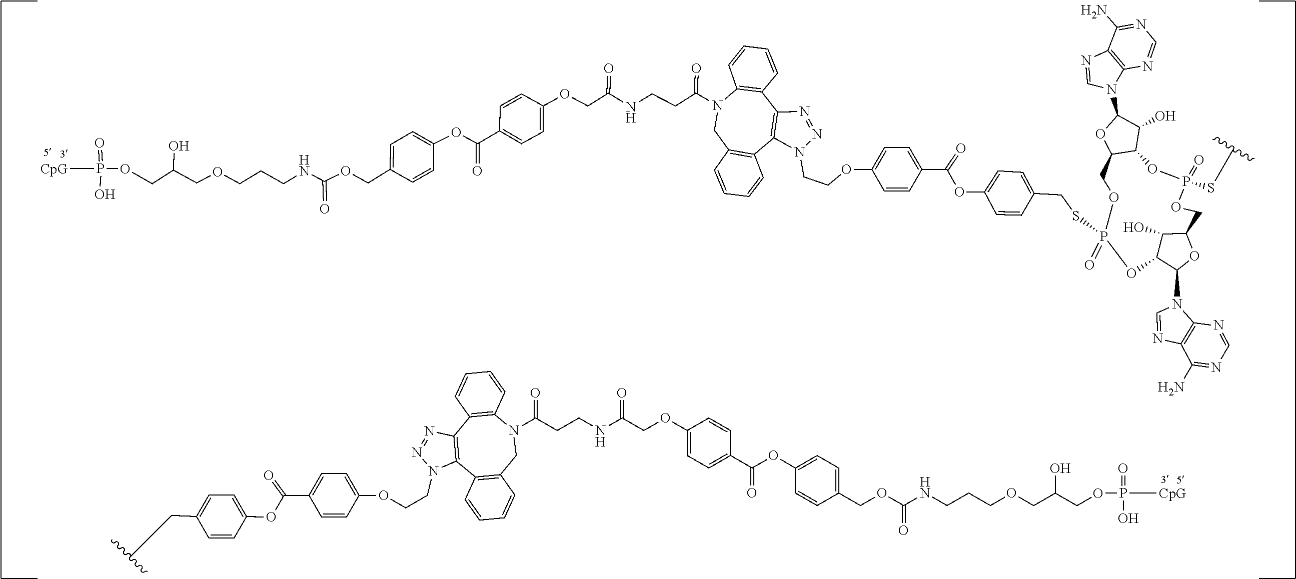

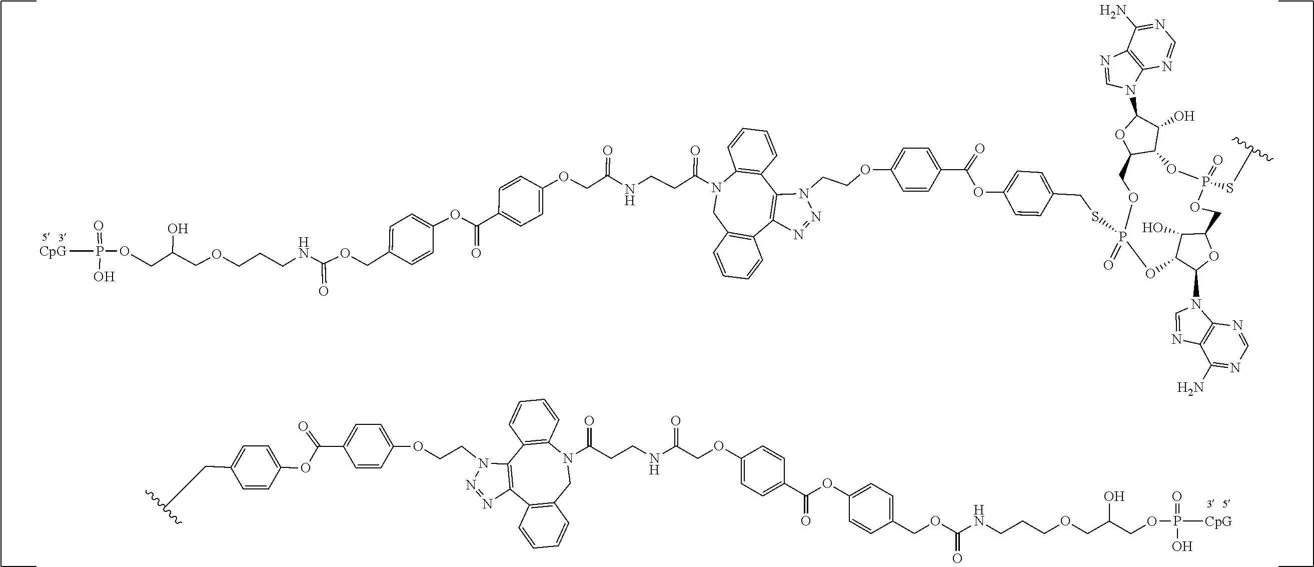

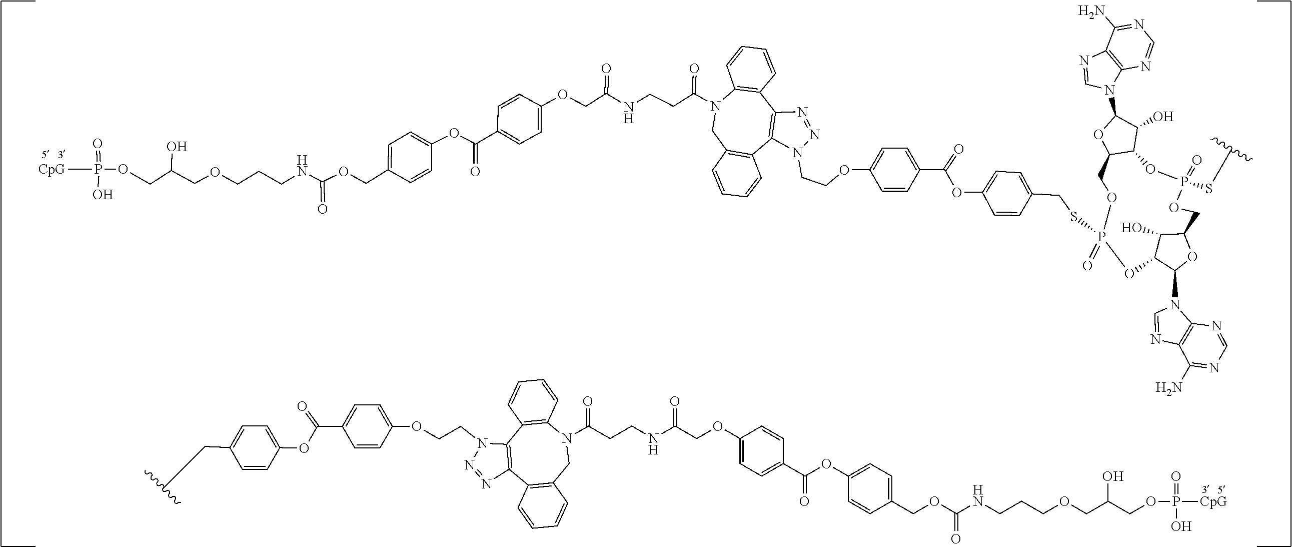

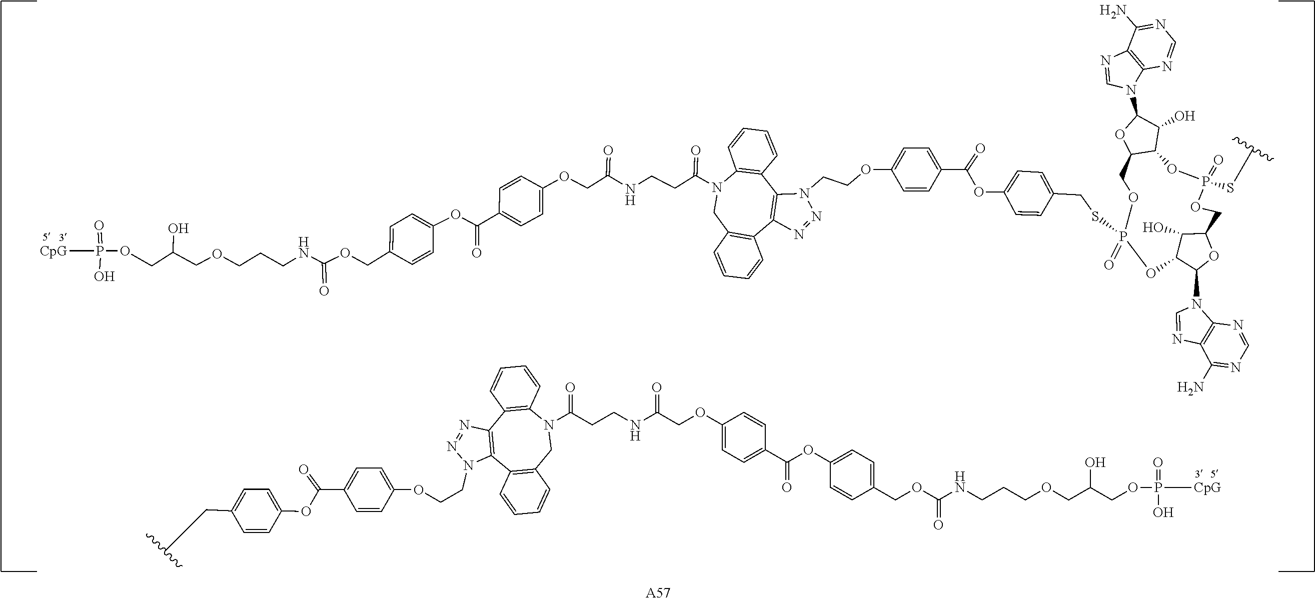

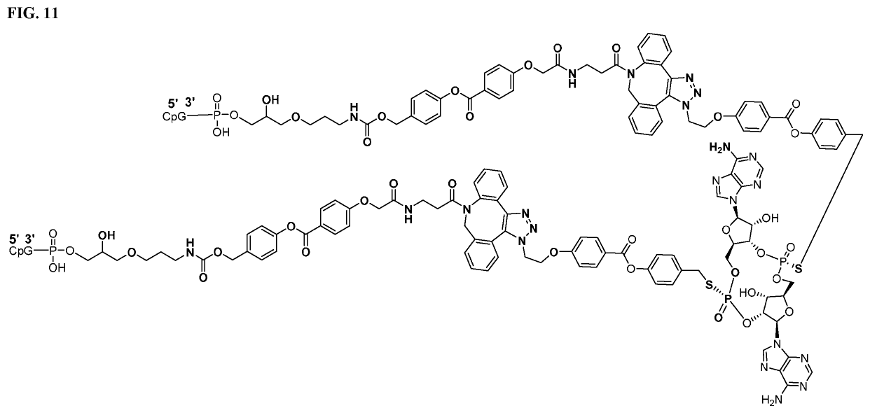

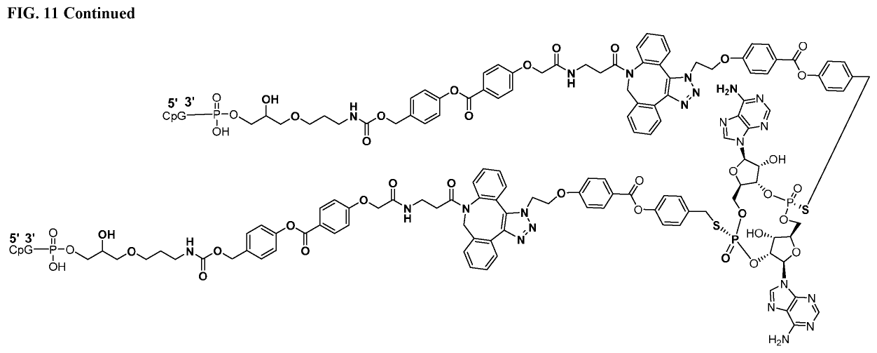

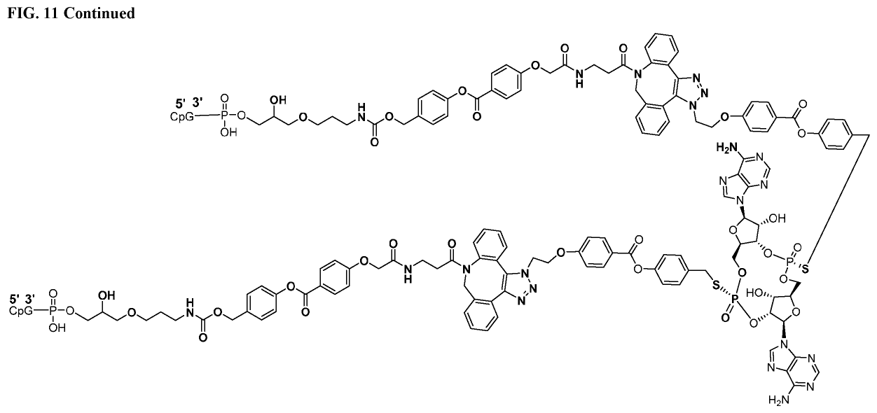

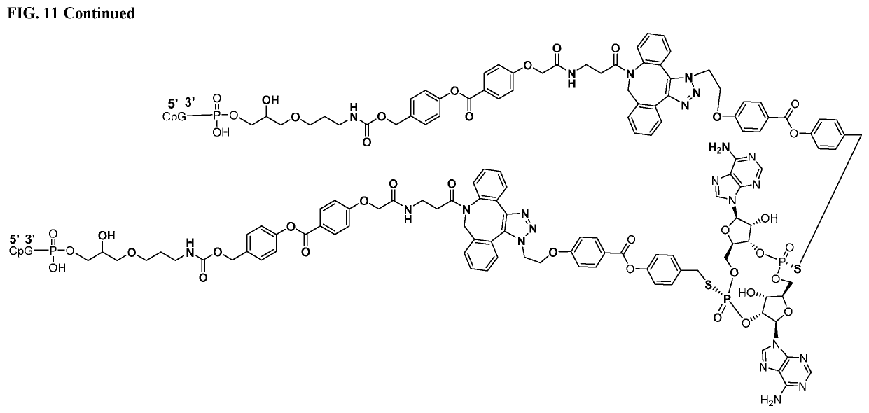

Each regioisomer in A57 depicted above inside brackets indicates that it is a single compound where there is a covalent bond between the two fragments indicated by [symbol] The full structure of A57 is shown in FIG. 11.

A mixture of regioisomers A57 is synthesized by the reaction of A15 with A52 according to the procedure described in Example 47.

### Example 58. Synthesis of A58 (a Mixture of Regioisomers as Shown Below)

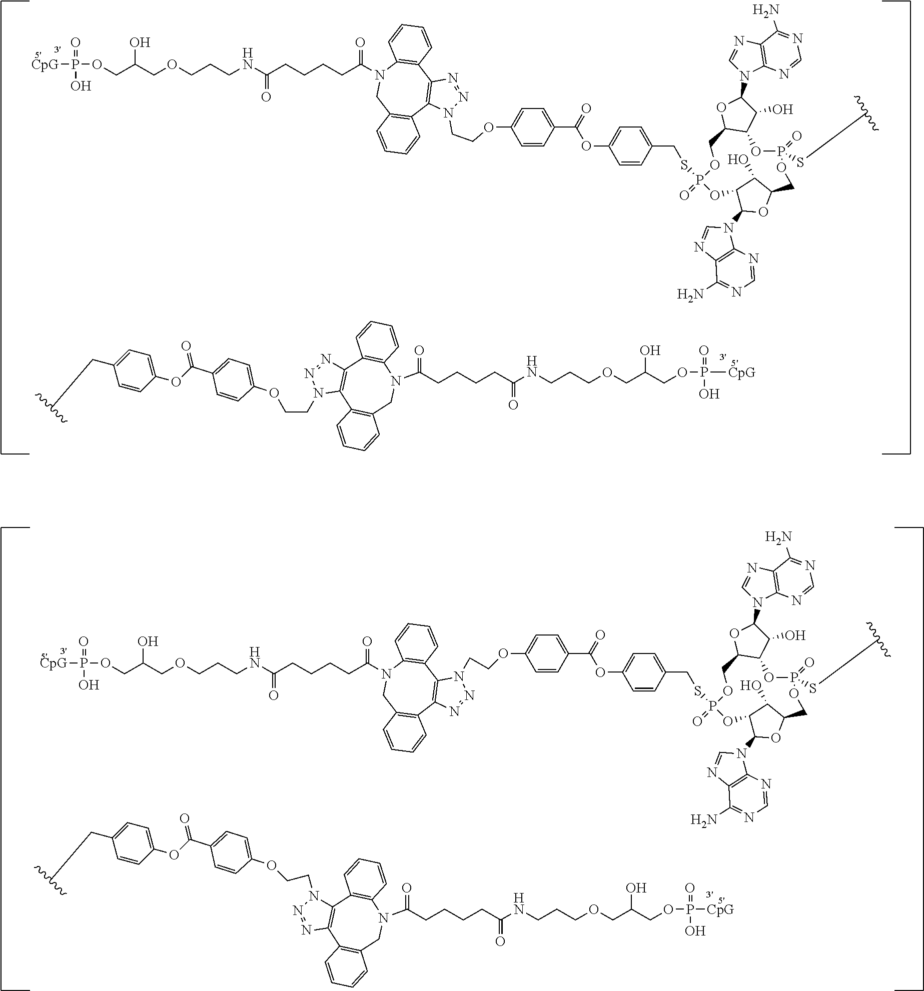

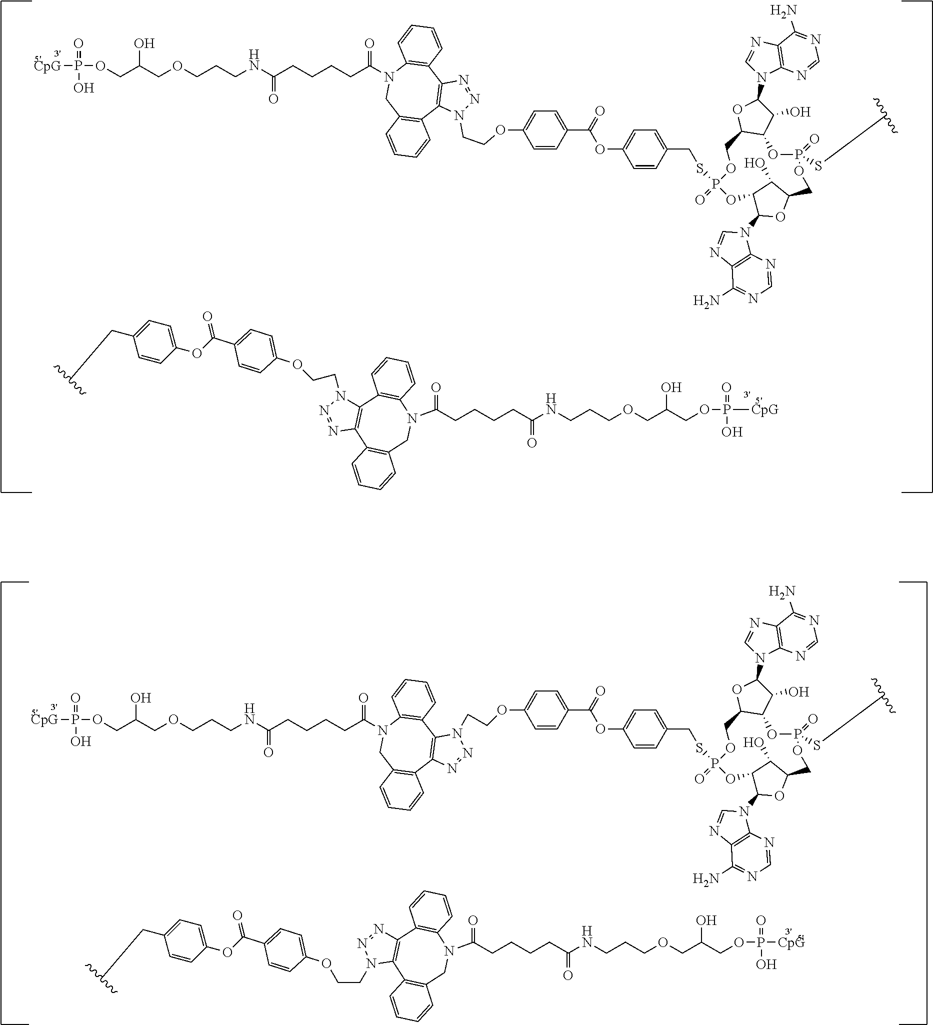

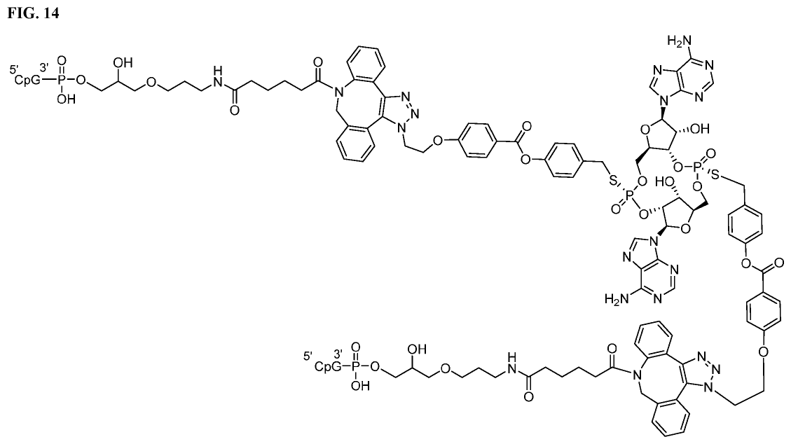

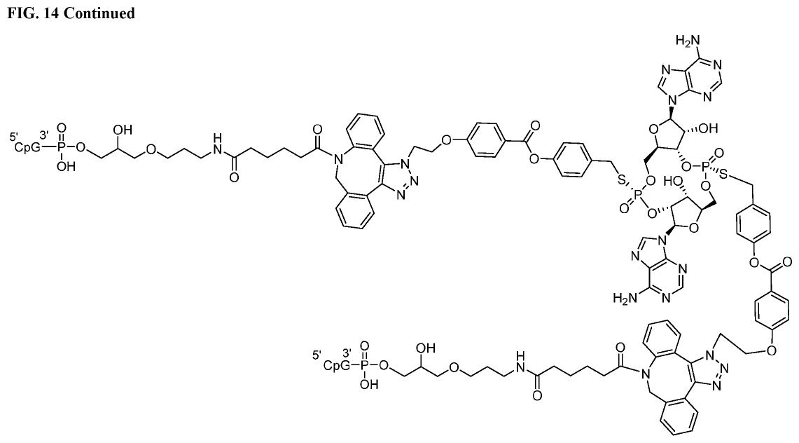

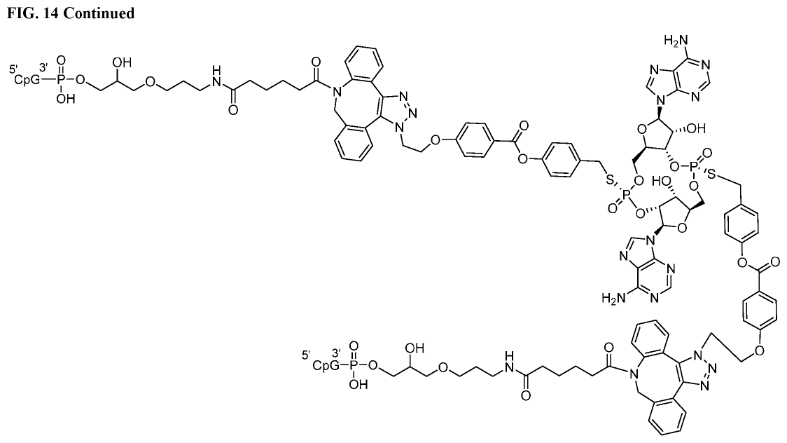

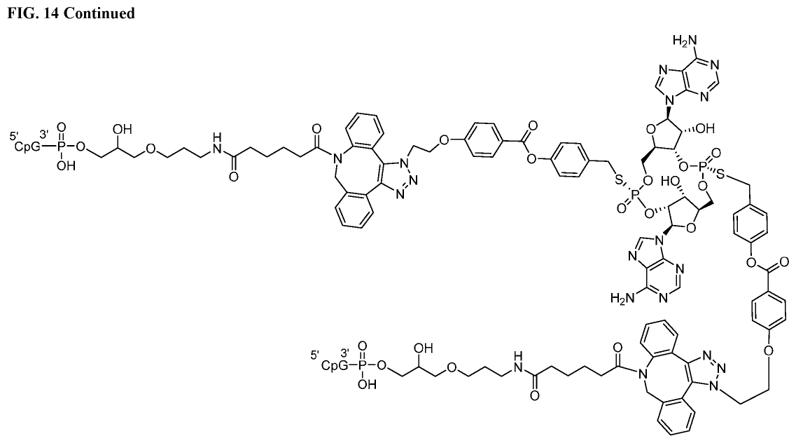

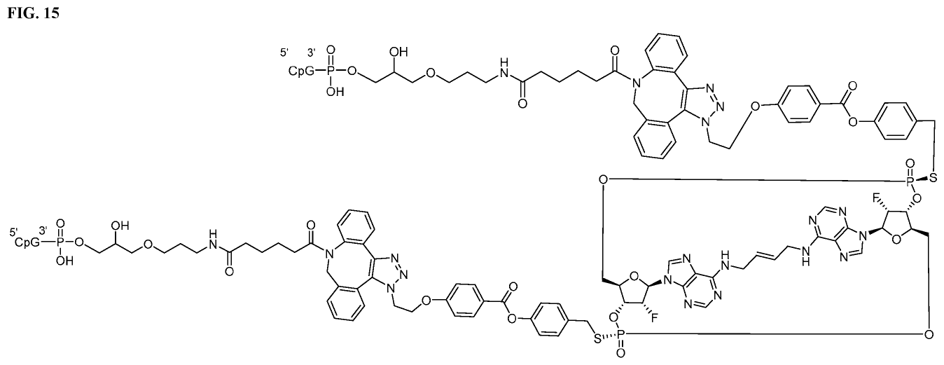

A mixture of regioisomers A58 (see FIG. 14) is synthesized by the reaction of A18 with A52 according to the procedure described in Example 50.

### Example 59. Synthesis of A59-I and A59-II

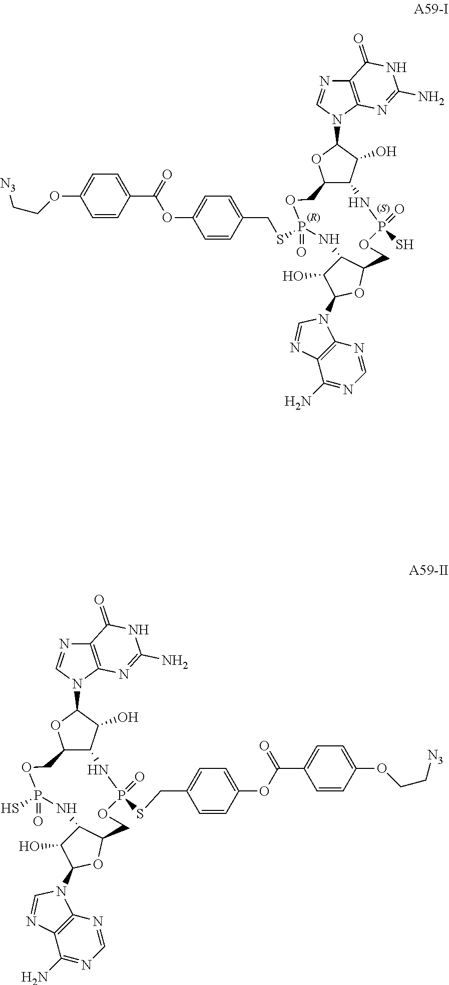

A59-I and A59-II are synthesized by the reaction of ammonium salt of 2-amino-9-((2R,3R,3aS,5S,7aS,9R,10R,10aS,12R,14aS)-9-(6-amino-9H-purin-9-yl)-3,10-dihydroxy-[3,2-d:3′,2′-j][1,7]dioxa[3,9]diaza [2,8]diphosphacyclododecin-2-yl)-1,9-dihydro-6H-purin-6-one (can be synthesized following the procedure described in WO 2020117739) with 4-(iodomethyl)phenyl 4-(2-azidoethoxy)benzoate according to the procedure described in Example 43.

### Example 60. Synthesis of A60

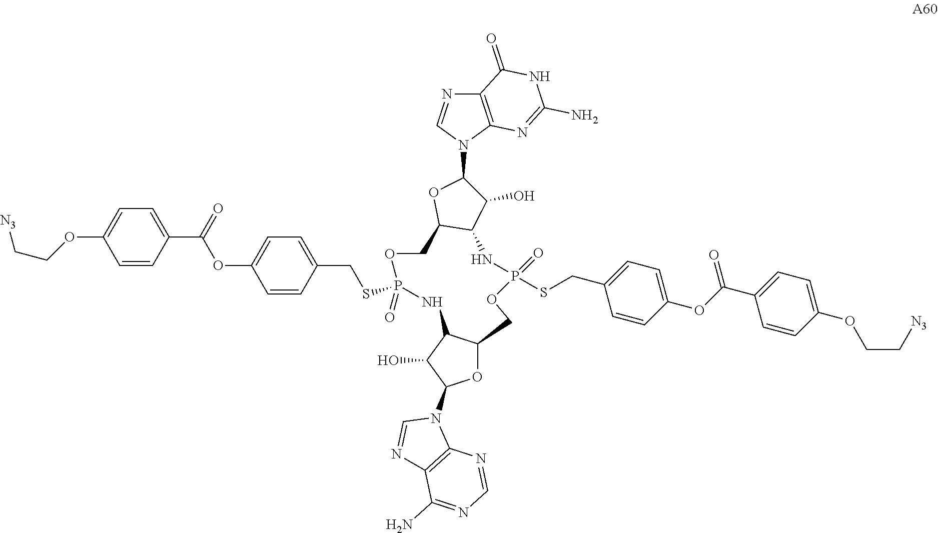

A60 is synthesized by the reaction of ammonium salt of 2-amino-9-((2R,3R,3aS,5S,7aS,9R,10R,10aS,12R,14aS)-9-(6-amino-9H-purin-9-yl)-3,10-dihydroxy-5,12-dimercapto-5,12-dioxidododecahydrodifuro[3,2-d:3′,2′-j][1,7]dioxa[3,9]diaza[2,8]diphosphacyclododecin-2-yl)-1,9-dihydro-6H-purin-6-one (can be synthesized following the procedure described in WO 2020117739) with 4-(iodomethyl)phenyl 4-(2-azidoethoxy)benzoate according to the procedure described in Example 46.

### Example 61. Synthesis of A61 (a Mixture of Regioisomers as Shown Below)

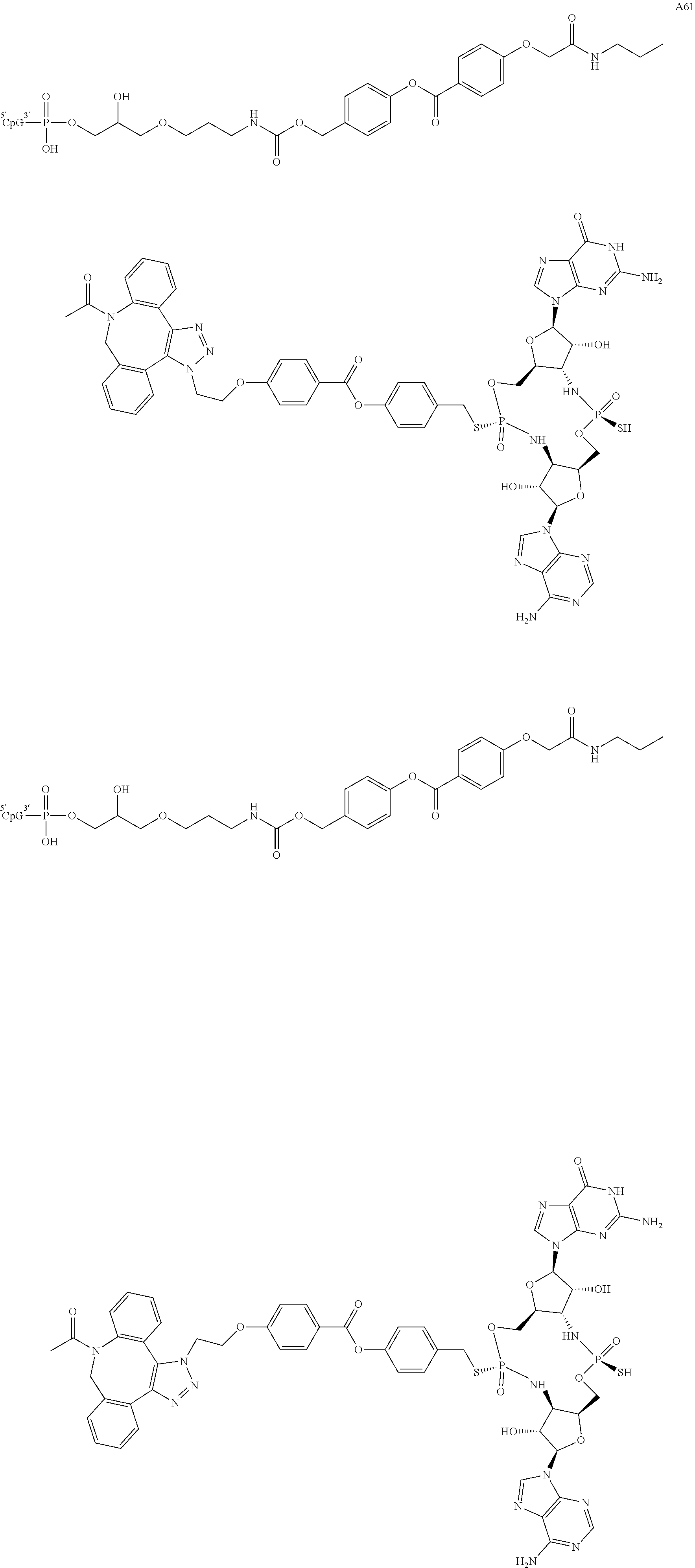

A mixture of regioisomers A61 is synthesized by the reaction of A15 with A59-I according to the procedure described in Example 16.

### Example 62. Synthesis of A62 (a Mixture of Regioisomers as Shown Below)

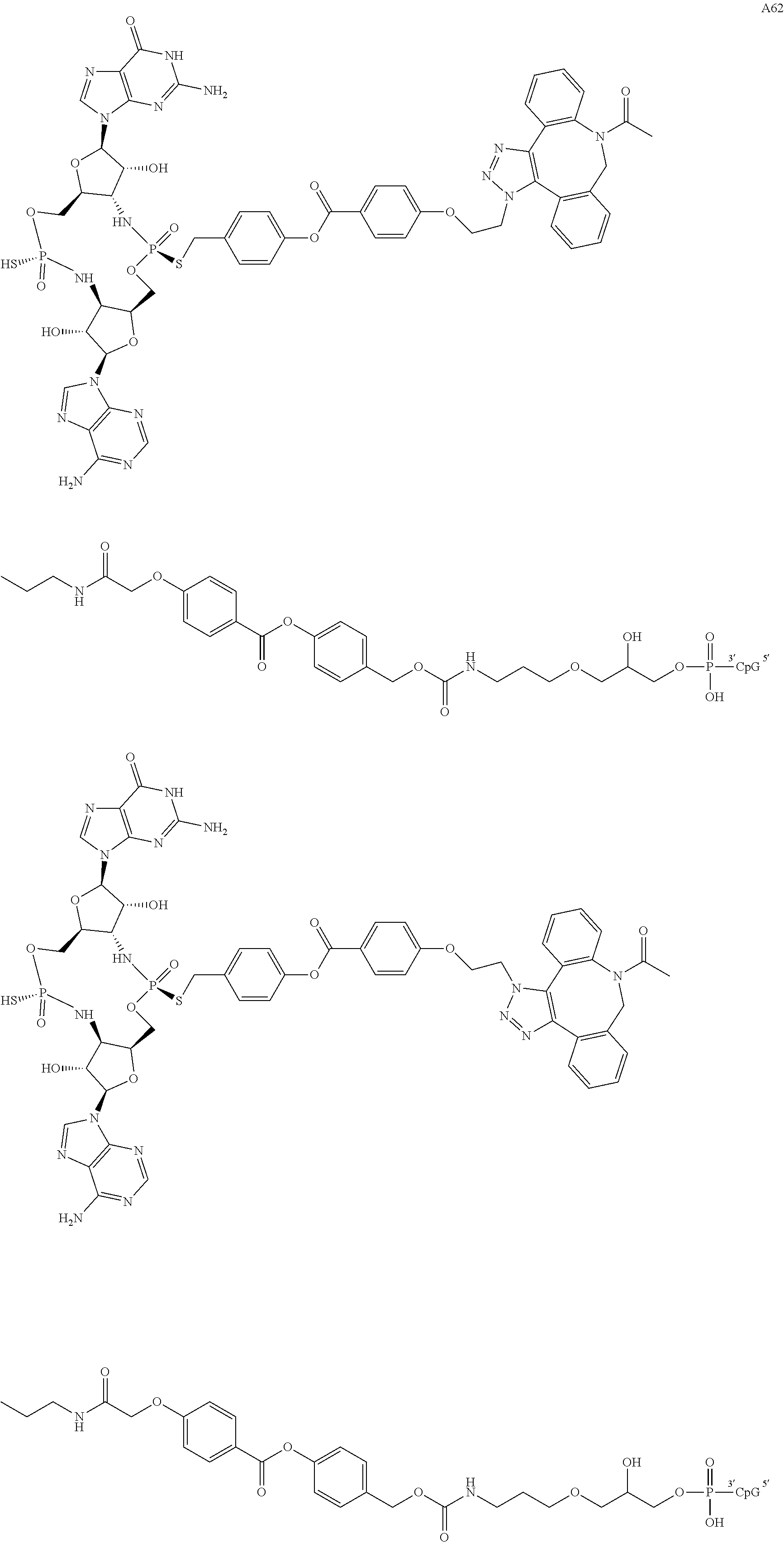

A mixture of regioisomers A62 is synthesized by the reaction of A15 with A59-II according to the procedure described in Example 16.

### Example 63. Synthesis of A63 (a Mixture of Regioisomers as Shown Below)

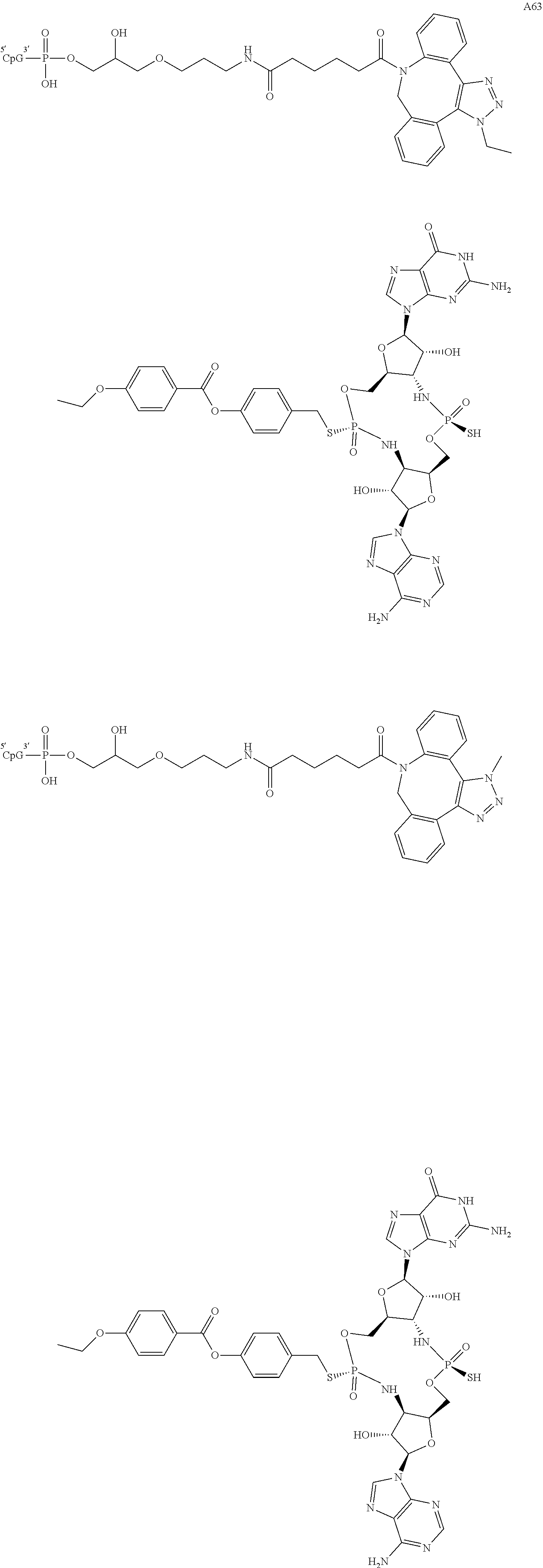

A mixture of regioisomers A63 is synthesized by the reaction of A18 with A59-I according to the procedure described in Example 19.

### Example 64. Synthesis of A64 (a Mixture of Regioisomers as Shown Below)

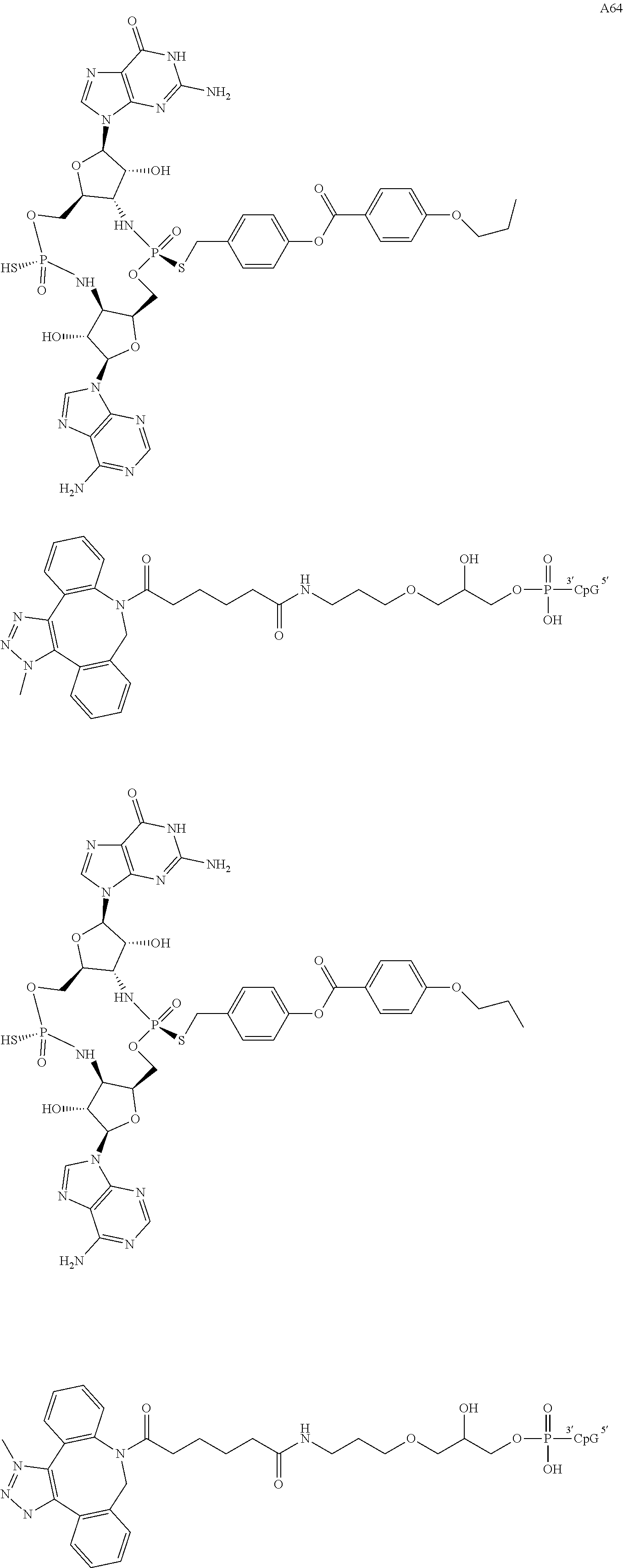

A mixture of regioisomers A64 is synthesized by the reaction of A18 with A59-II according to the procedure described in Example 19.

### Example 65. Synthesis of A65 (a Mixture of Regioisomers as Shown Below)

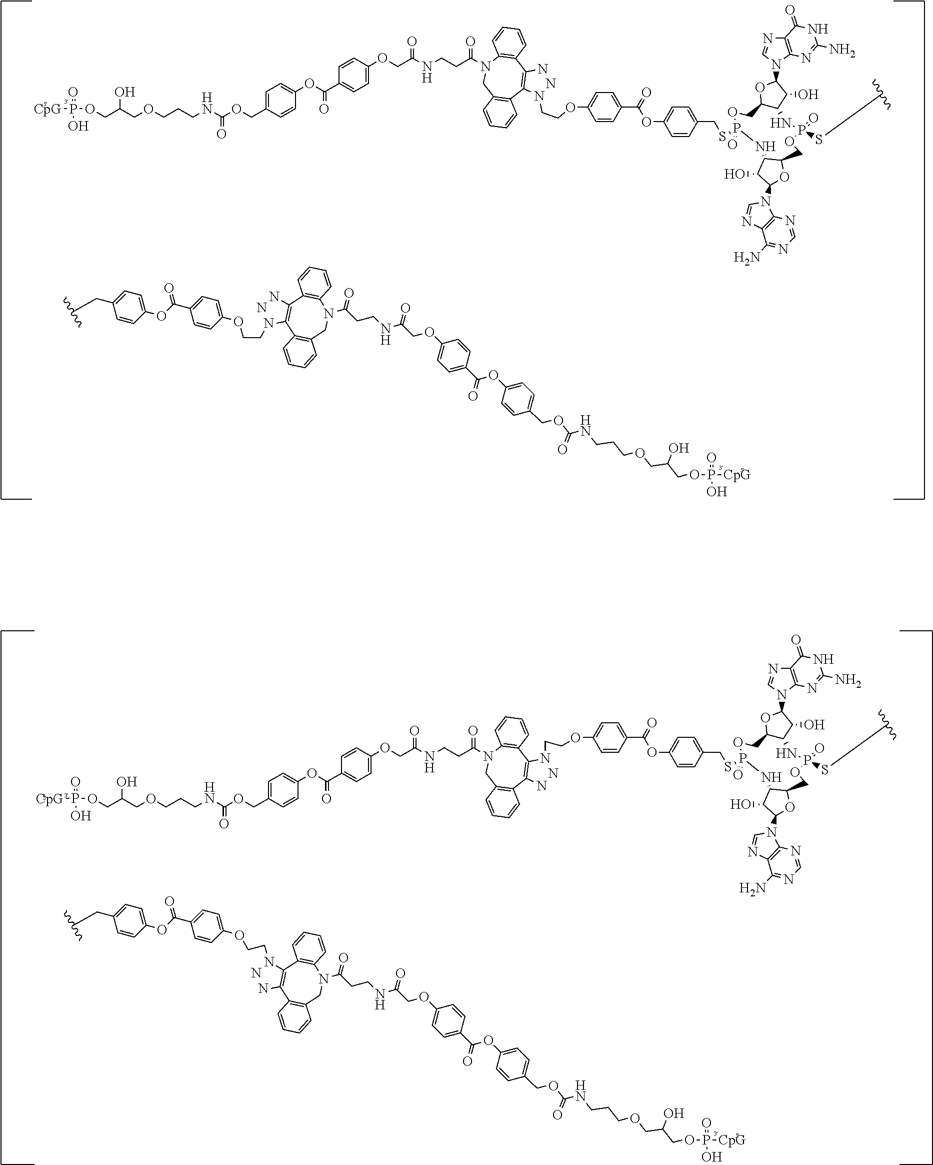

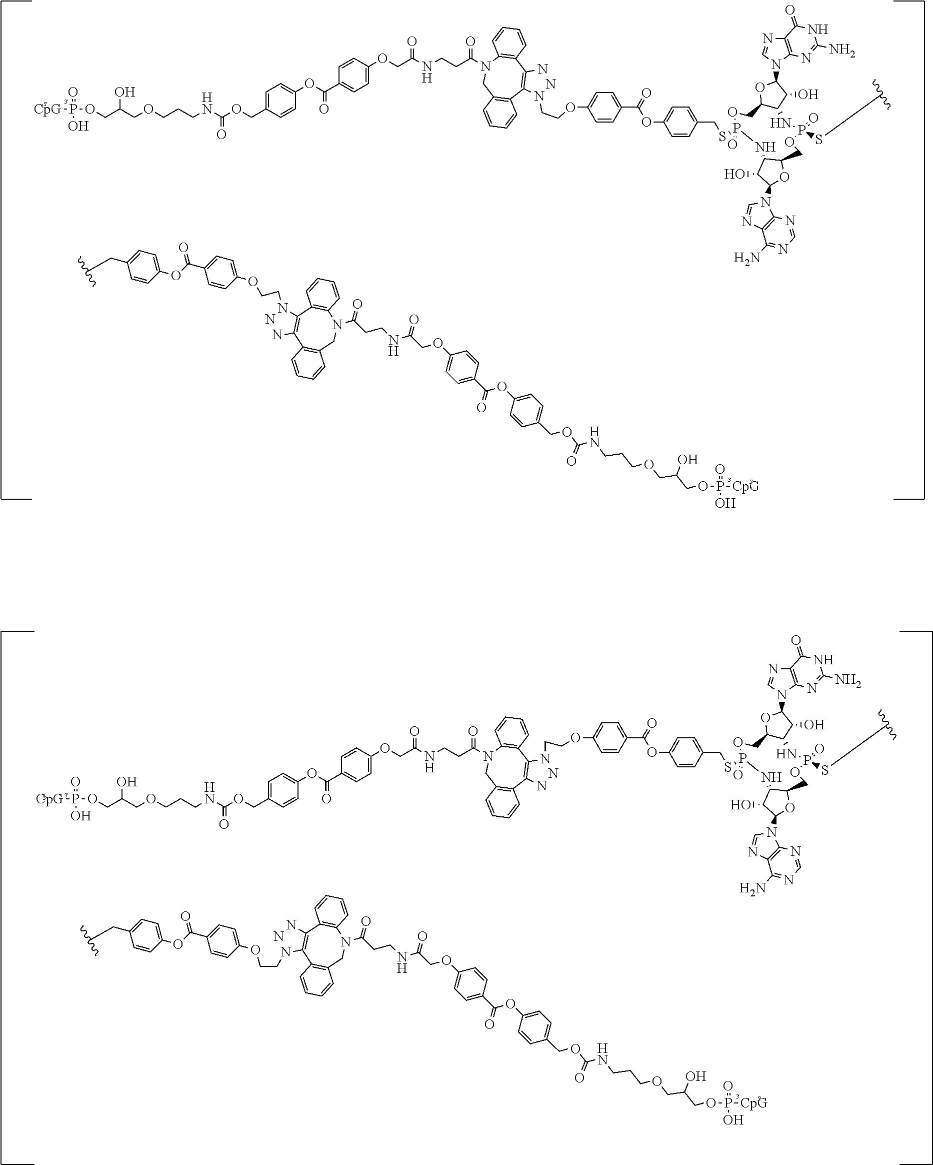

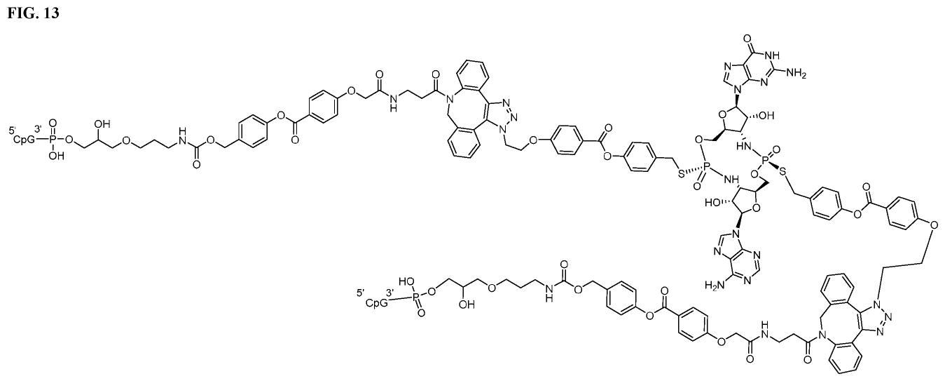

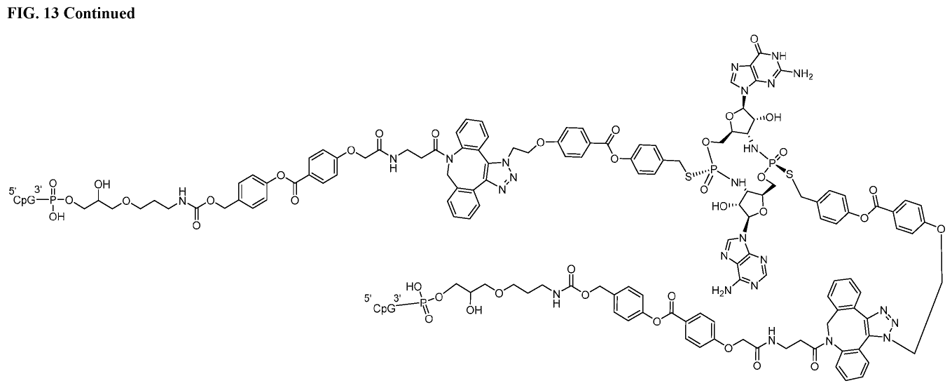

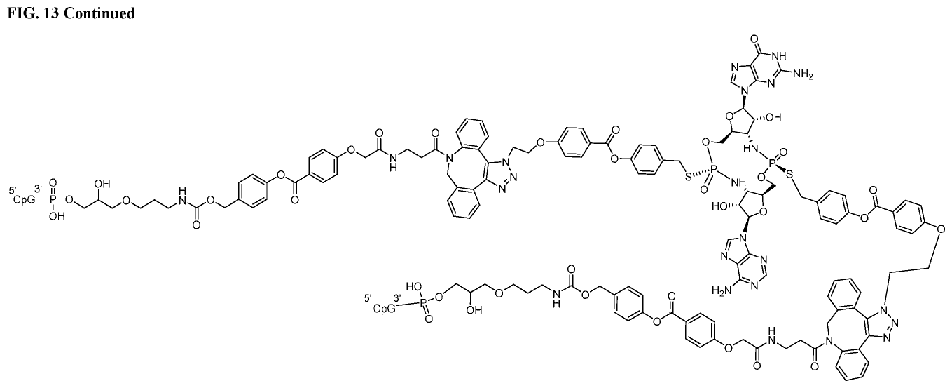

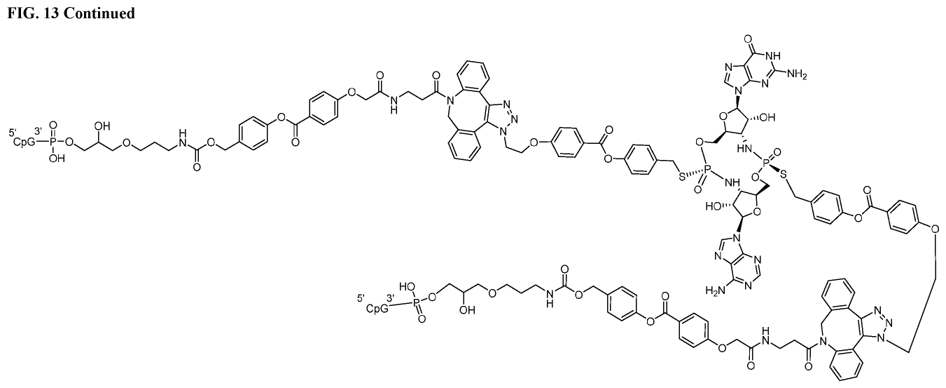

A mixture of regioisomers A65 (see FIG. 13) is synthesized by the reaction of A15 with A60 according to the procedure described in Example 47.

### Example 66. Synthesis of A66 (a Mixture of Regioisomers as Shown Below)

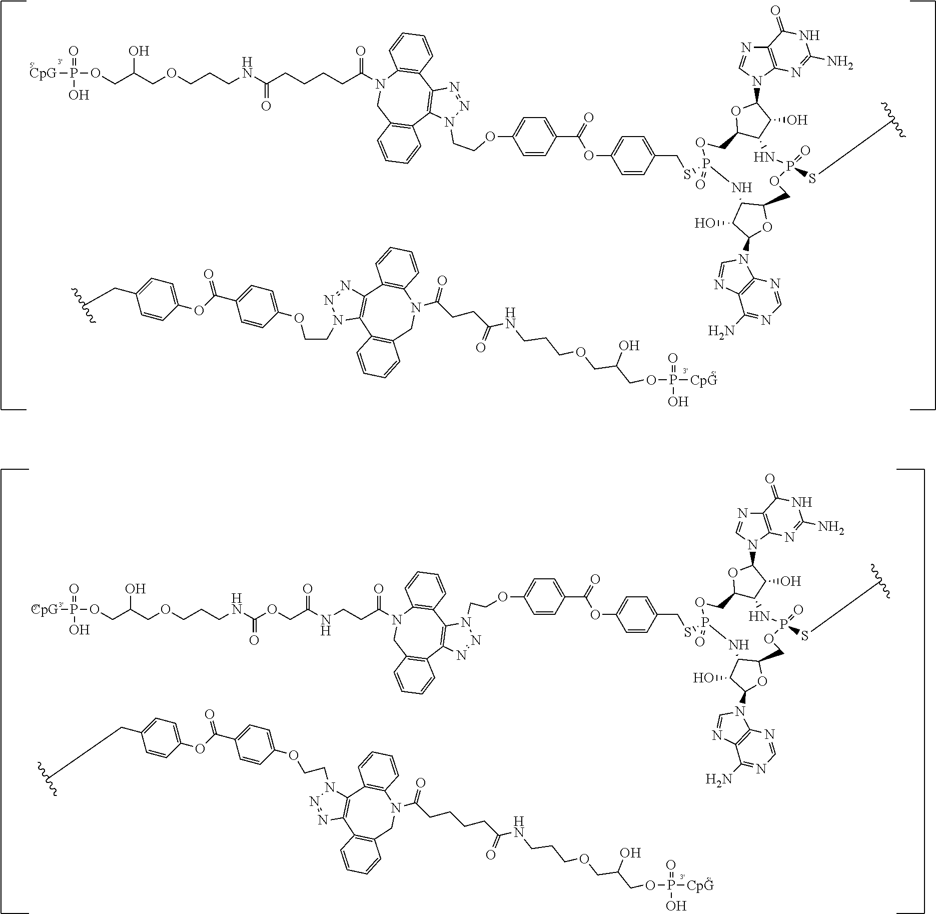

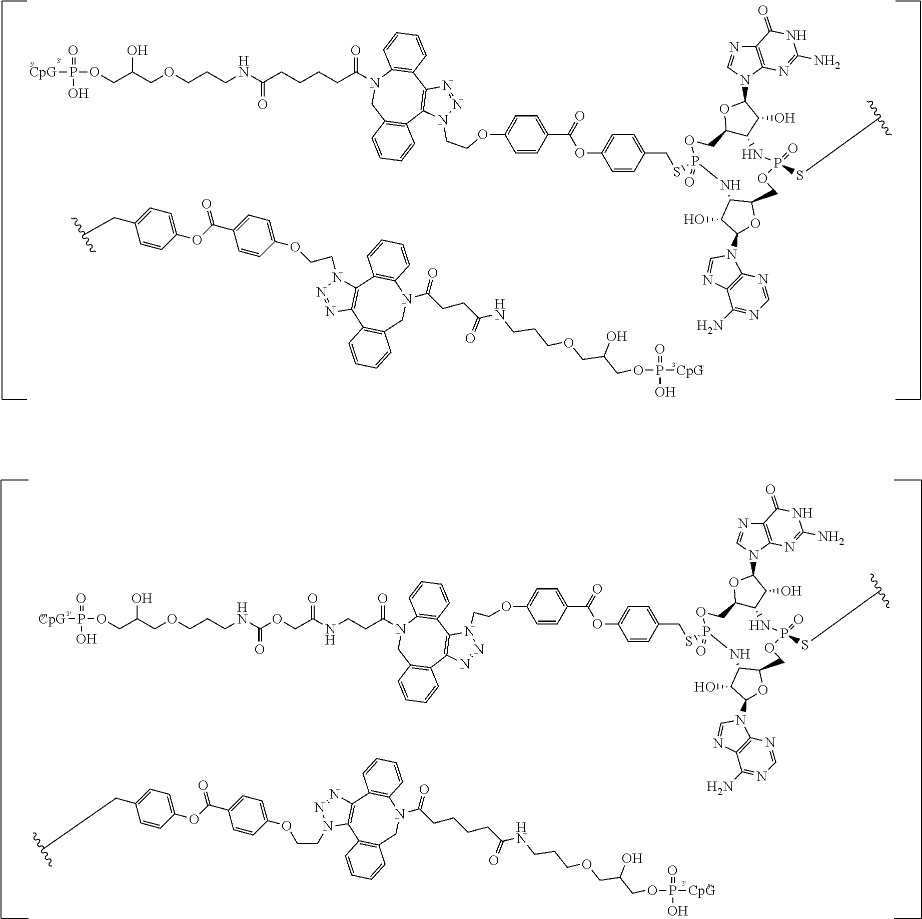

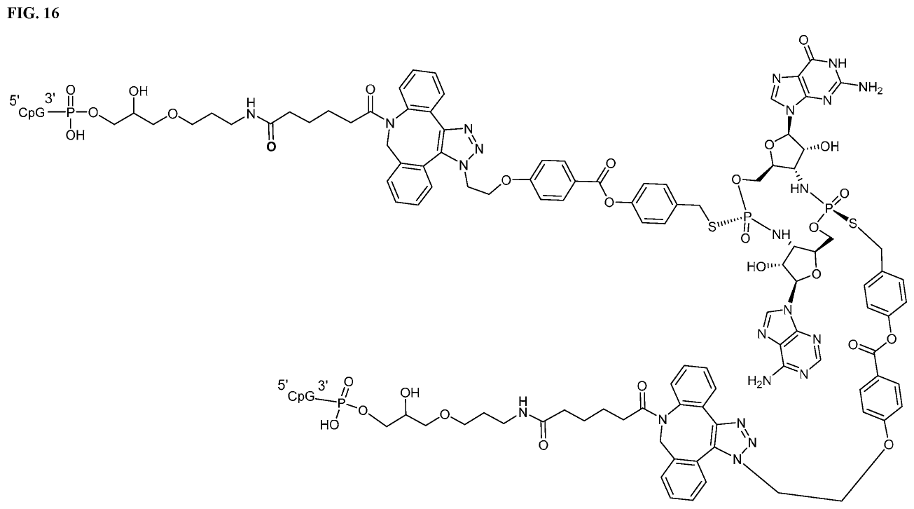

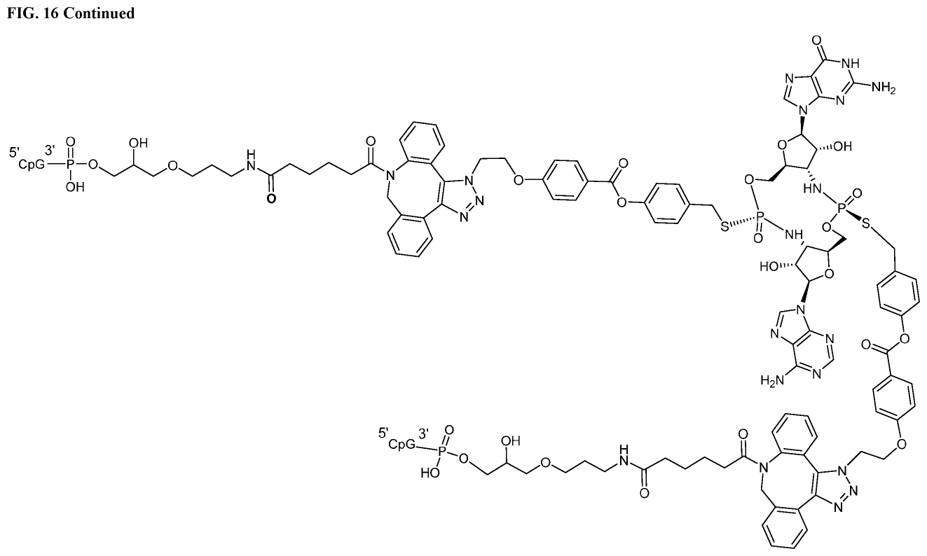

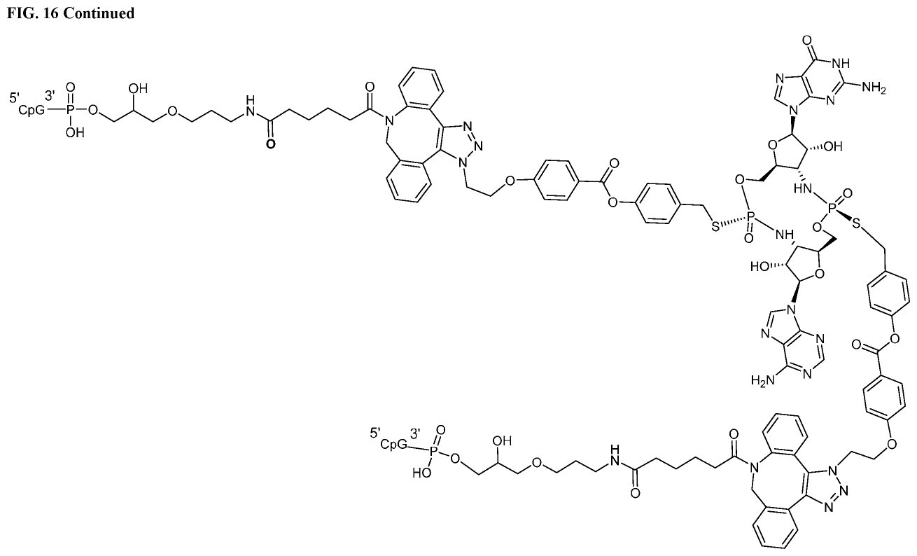

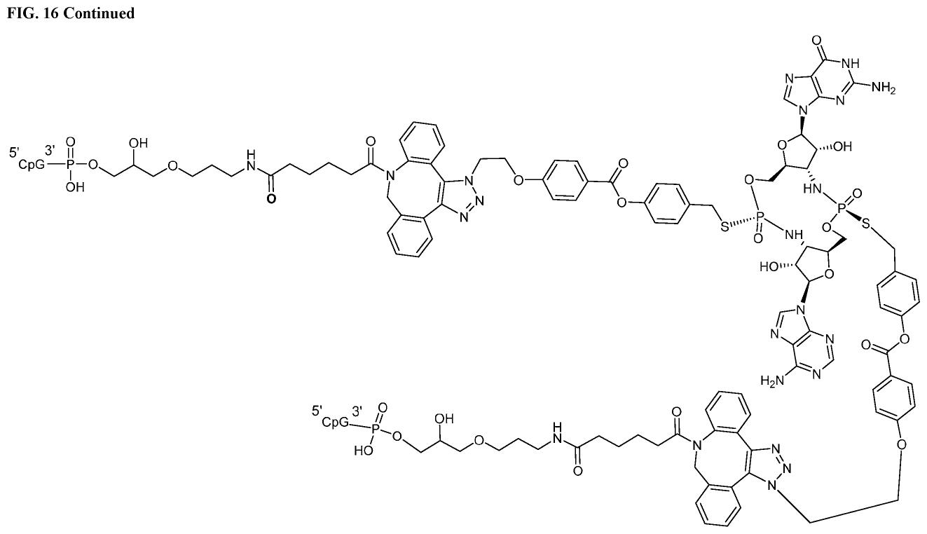

A mixture of regioisomers A66 (see FIG. 16) is synthesized by the reaction of A18 with A60 according to the procedure described in Example 50.

### Example 67. Synthesis of A67

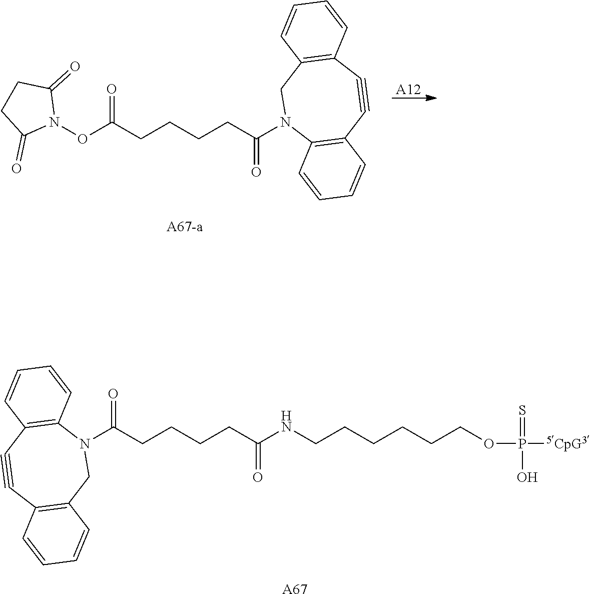

A12 (82.0 mg, 8.31 umol, 1.00 eq) in PBS (2 mL, pH=8) was added to a solution of A67-a (35.7 mg, 83.1 umol, 10.0 eq) in DMF at 25° C. The solution was stirred at rt for 2 hrs. The reaction mixture was extracted with DCM (10.0 mL), the water layer was concentrated under reduced pressure to give a residue, which was washed with pure water (30.0 mL; 10.0 mL×3) and filtered. The aqeuous layers were concentrated under reduced pressure to give a solid (87.9 mg, crude), which was used in the next step. LC/MS calculated mass 10182 Da, measured mass 10182 Da.

### Example 68. Synthesis of A68 (a Mixture of Regioisomers as Shown Below)

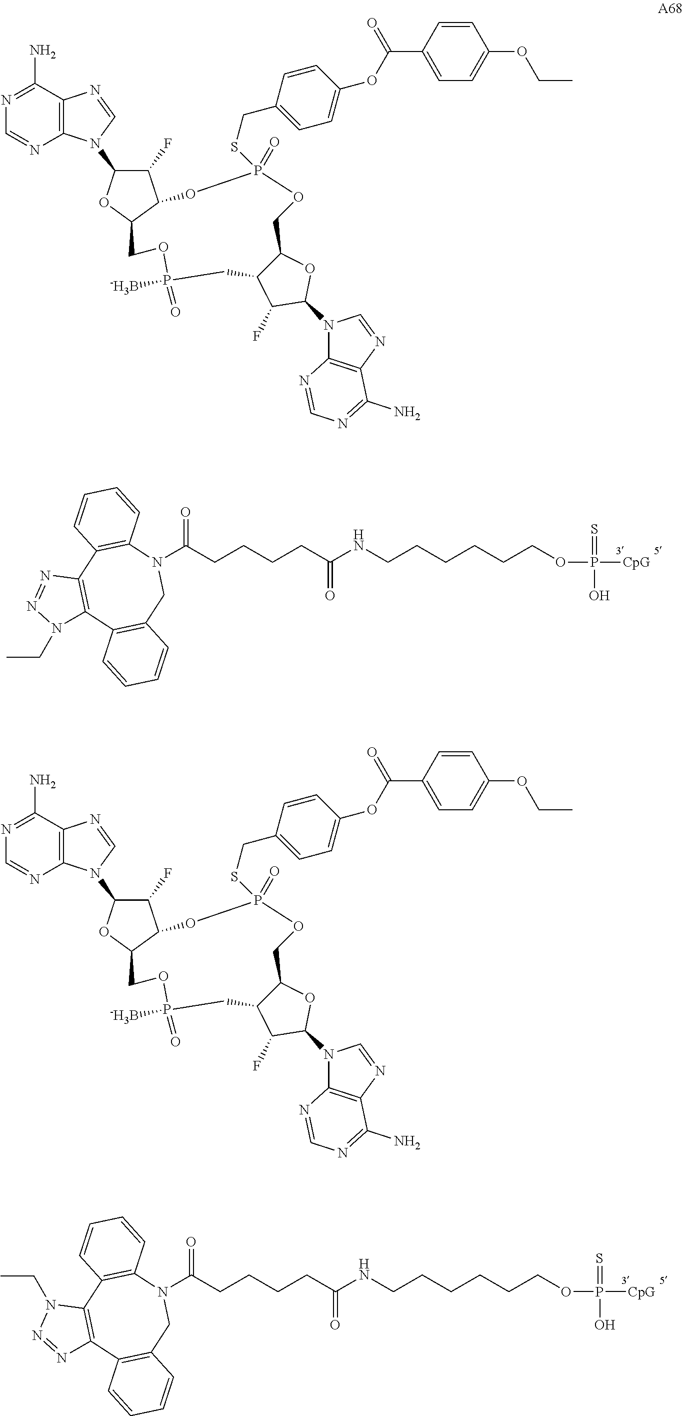

A mixture of regioisomers A68 was synthesized by the reaction of A67 with A7 according to the procedure described in Example 19. Yield: 17.7%, Purity 95.8%. Clarity RP C18 3 um 4.6*100 mm, RT=13.27 min. Calculated mass 11152 Da, measured mass 11153 Da.

### Example 69. Synthesis of A69 (a Mixture of Regioisomers as Shown Below)

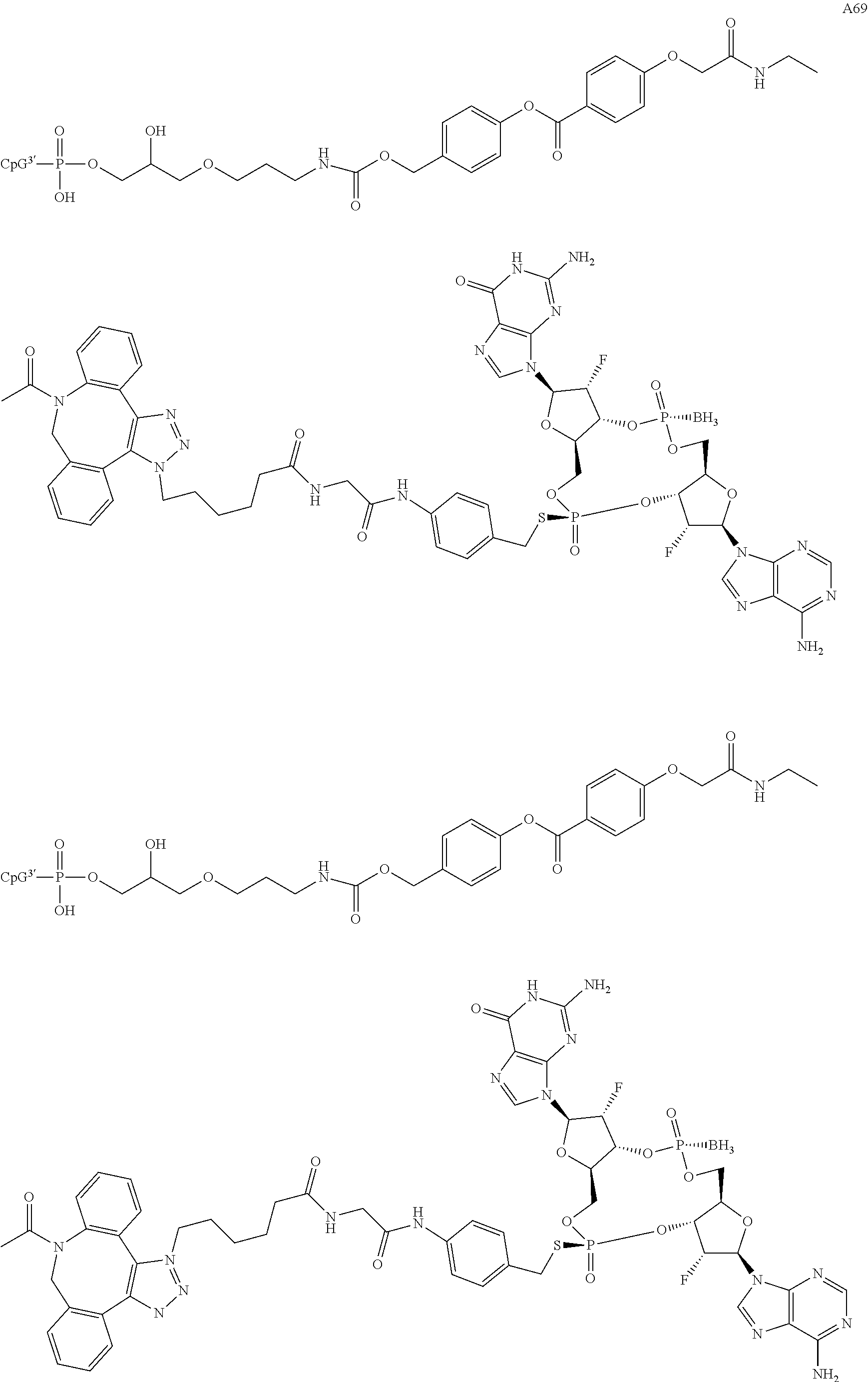

A mixture of regioisomers A69 was synthesized by the reaction of A15 with A4 according to the procedure described in Example A33. Yield: 25.3%, Purity 75%. Clarity RP C18 3 um 4.6*100 mm, RT=13.85 min. Calculated mass 11462 Da, measured mass 11462 Da.

### Example 70 Synthesis of ((2R,3R,3aR,5 S,7aR,9R,10R,10aR,12S,14aR)-2,9-bis(6-amino-9H-purin-9-yl)-12-((4-((2-(2-(2-azidoethoxy)ethoxy)ethoxy)methyl)benzyl)thio)-3,10-difluoro-5,12-dioxidooctahydro-2H,7H-difuro[3,2-d:3′,2′-j][1,3,7,9]tetraoxa [2,8]diphosphacyclododecin-5-yl)trihydroborate

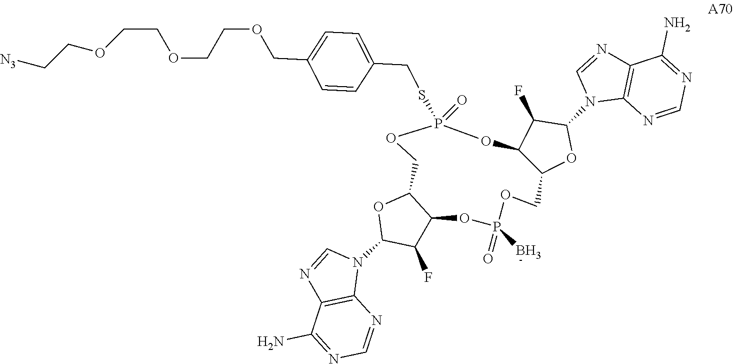

To a solution of I-16 (65.8 mg, 209 umol, 1.00 eq) in acetone (0.5 mL) and THF (0.5 mL), was added KI (139 mg, 838 umol, 4.00 eq) at 25° C. and the mixture was stirred at 25° C. for 1 hr. Then a solution of compound A5-I sodium salt (59.0 mg, 81.7 umol, 0.500 eq) in DMF (0.5 mL) was added and stirred at 25° C. for 20 hrs. The reaction mixture was purified by prep-HPLC (neutral condition, column: YMC Triart C18 150*25 mm*5 um; mobile phase: [water (10 mM NH₄HCO₃)-ACN]; B %: 10%-40%, 10 min) to give compound A70 (31.0 mg, 32.5 umol, 39.7% yield) as a solid. LC-MS (ES, m/z): 952.1 [M−H]⁺, RT=1.052 min. ¹H NMR (400 MHz, DMSO-d₆) 8.51 (s, 1H), 8.23 (s, 1H), 8.19 (s, 1H), 8.06 (s, 1H), 7.14 (s, 4H), 6.53-6.13 (m, 2H), 5.90-5.34 (m, 4H), 4.43 (br d, J=14.8 Hz, 2H), 4.38 (s, 2H), 4.35-4.22 (m, 2H), 4.14 (br d, J=11.6 Hz, 1H), 3.89 (br dd, J=3.2, 16.8 Hz, 2H), 3.79 (br d, J=8.4 Hz, 1H), 3.59-3.56 (m, 3H), 3.52 (br d, J=1.6 Hz, 7H), 3.49 (br s, 3H), 3.38-3.28 (m, 2H), 2.67 (s, 1H), 2.33 (br s, 1H), 0.25 (br s, 3H).

### Example 71. Synthesis of A71 (a Mixture of Regioisomers as Shown Below)

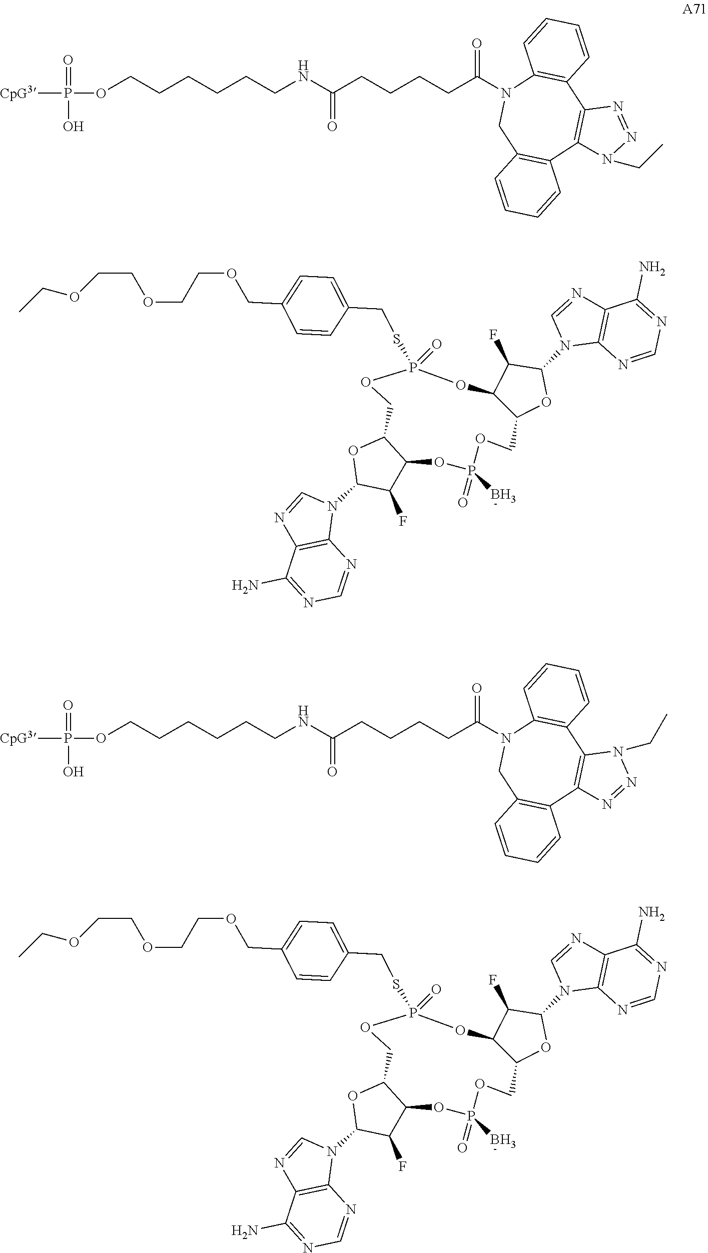

A mixture of regioisomers A71 was synthesized by the reaction of A18 with A70 according to the procedure described in Example A33. Yield: 23.5%, Purity 95.5%. Clarity RP C18 3 um 4.6*100 mm, RT=13.04 min. Calculated mass 11150 Da, measured mass 11151 Da.

### Example 72. Synthesis of A72

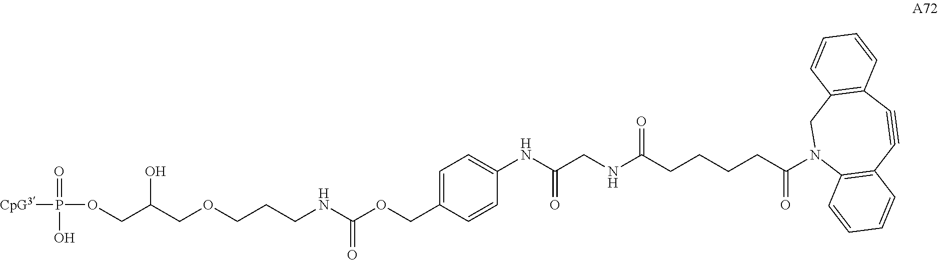

### Step 1. Synthesis of Compound A72-a

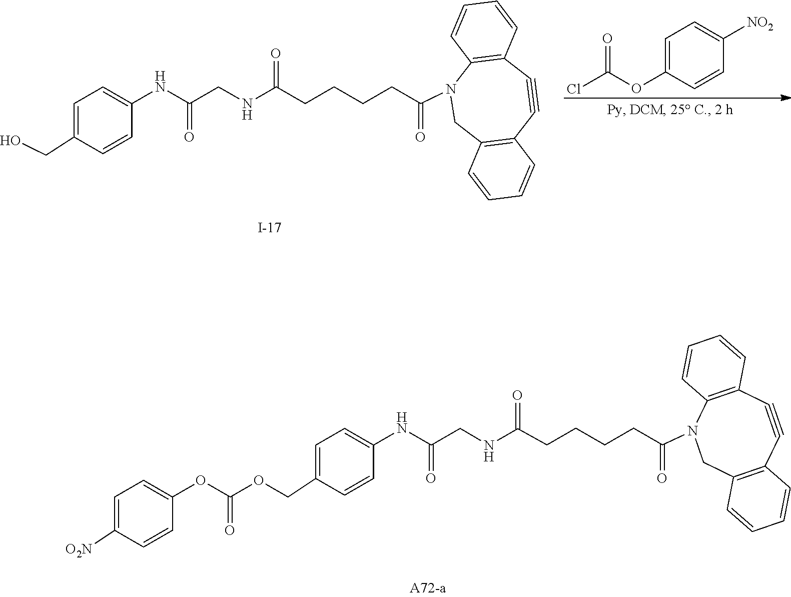

To a suspension of I-17 (57.0 mg, 115 umol, 1.00 eq) in DCM (1 mL) was added 4-nitrophenyl carbonochloridate (46.3 mg, 230 umol, 2.00 eq) followed by pyridine (36.4 mg, 460 umol, 37.1 uL, 4.00 eq) at 20° C. under N₂. The mixture was stirred at 25° C. for 2 hrs. The reaction solution was purified by prep-TLC (SiO₂, DCM:MeOH=10:1) to give A72-a (54.0 mg, crude) as an oil. LC-MS [M+H]⁺ 661.5.

### Step 2. Synthesis of A72

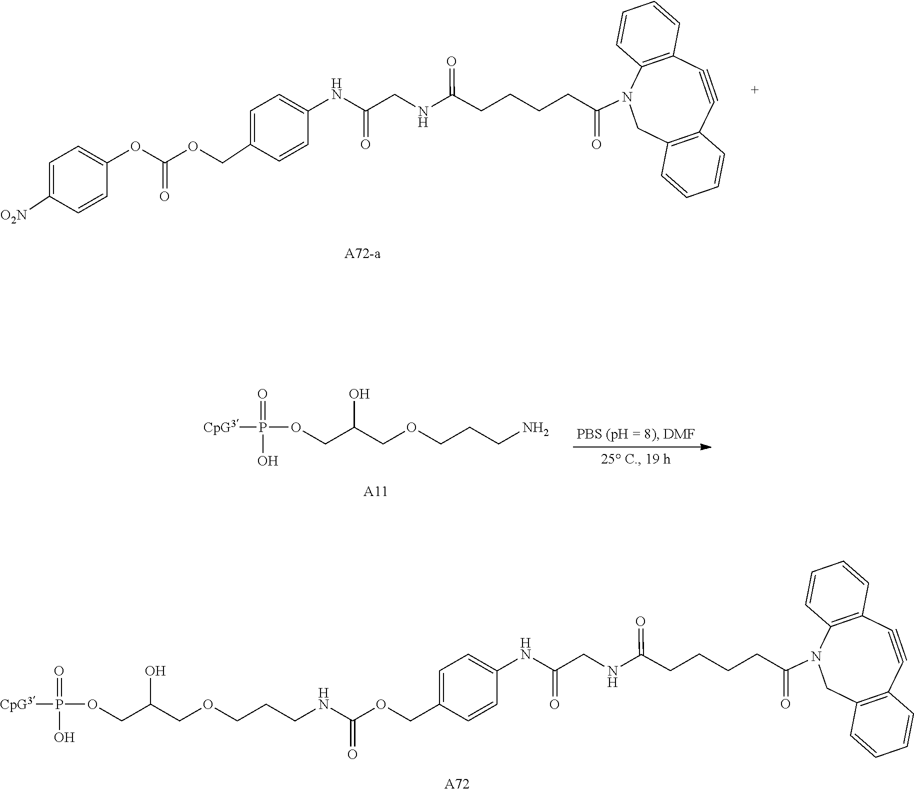

A11 (80.0 mg, 8.09 umol, 1.00 eq) in PBS (0.5 mL, pH=8) was added to a solution of A72-a (53.5 mg, 80.9 umol, 10.0 eq) in DMF (1 mL) at 25° C. and stirred for 19 hrs. The solution was concentrated to give an oil, which was diluted with H₂O (10 mL), the solution was extracted with DCM (10 mL), the aqeuous layer was concentrated to give a solid (84.0 mg, crude). Calculated mass 10404 Da, measured mass 10404 Da.

### Example 73. Synthesis of A73 (a Mixture of Regioisomers as Shown Below)

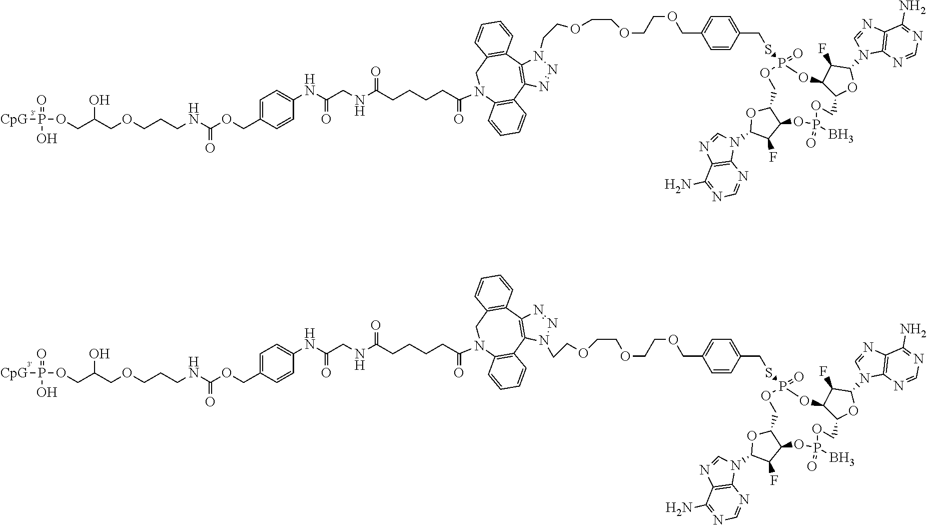

A mixture of regioisomers A73 was synthesized by the reaction of A72 with A70 according to the procedure described in Example A33. Yield: 16.1%, Purity 97.9%. Clarity RP C18 3 um 4.6*100 mm, RT=12.33 min. Calculated mass 11357 Da, measured mass 11358 Da.

### Example 74. Synthesis of A74 (a Mixture of Regioisomers as Shown Below)

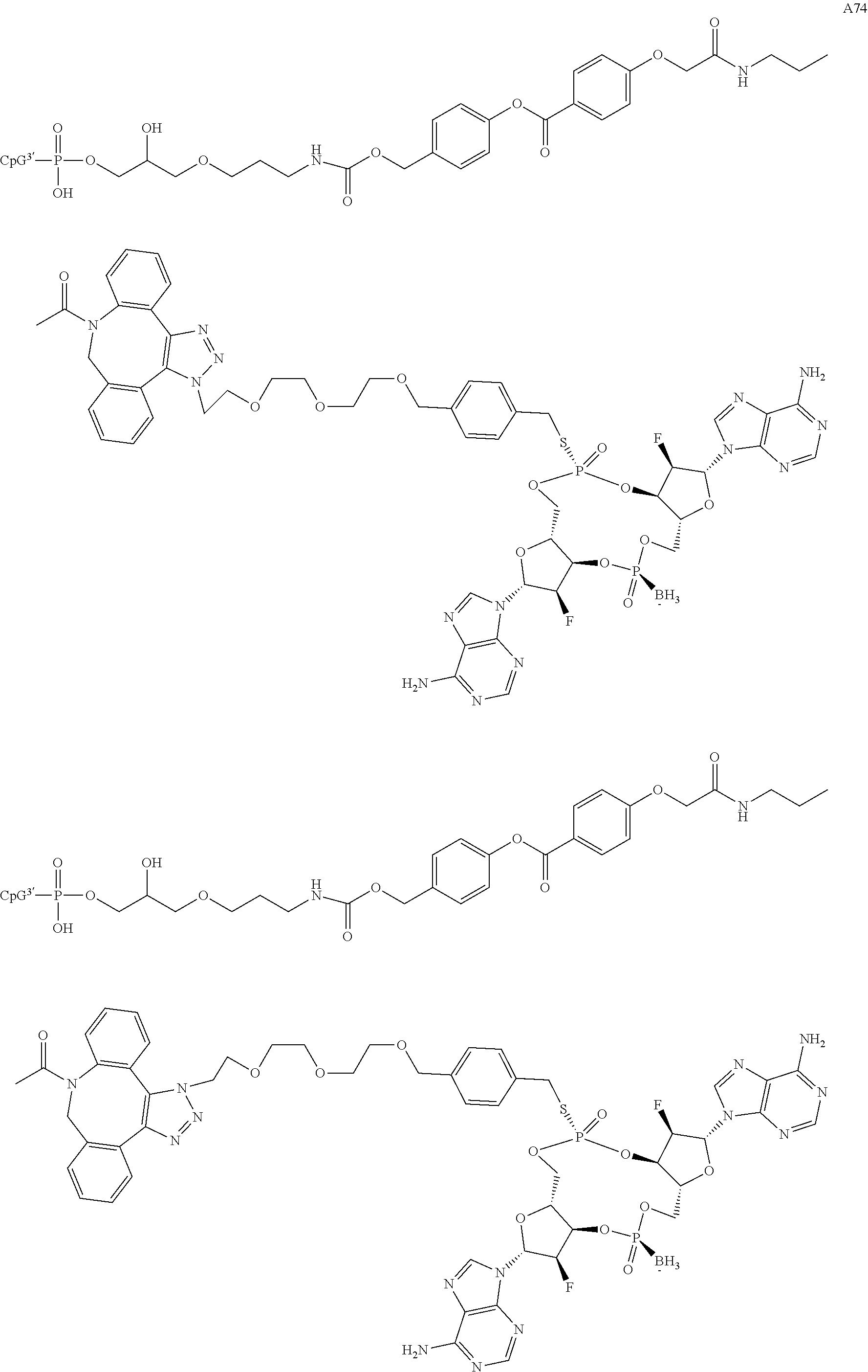

A mixture of regioisomers A74 was synthesized by the reaction of A15 with A70 according to the procedure described in Example A33. Yield: 19.1%, Purity 91.8%. Clarity RP C18 3 um 4.6*100 mm, RT=17.39 min. Calculated mass 11422 Da, measured mass 11424 Da.

### Example 75. Synthesis of A75 (a Mixture of Regioisomers as Shown Below)

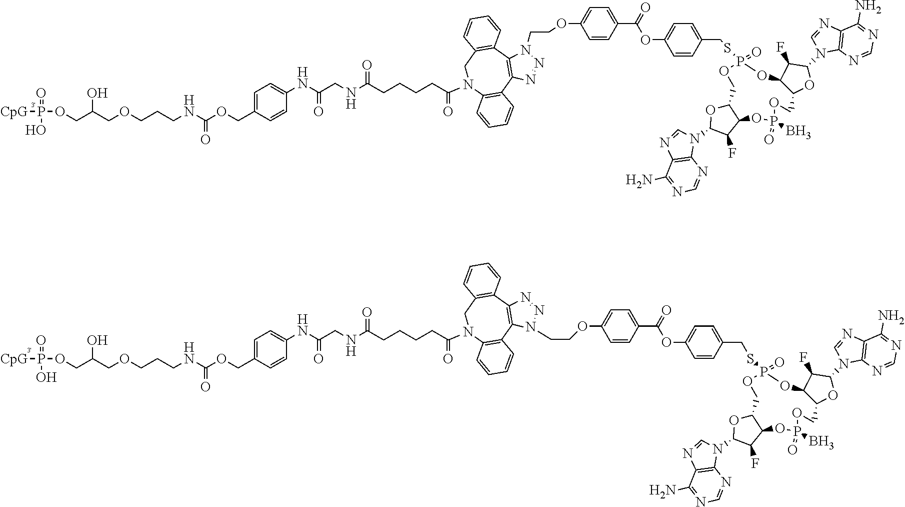

A mixture of regioisomers A75 was synthesized by the reaction of A72 with A7 according to the procedure described in Example A33. Yield: 12.2%, Purity 93.5%. Clarity RP C18 3 um 4.6*100 mm, RT=15.06 min. Calculated mass 11375 Da, measured mass 11377 Da.

### Example 76. Synthesis of A76 (a Mixture of Regioisomers as Shown Below)

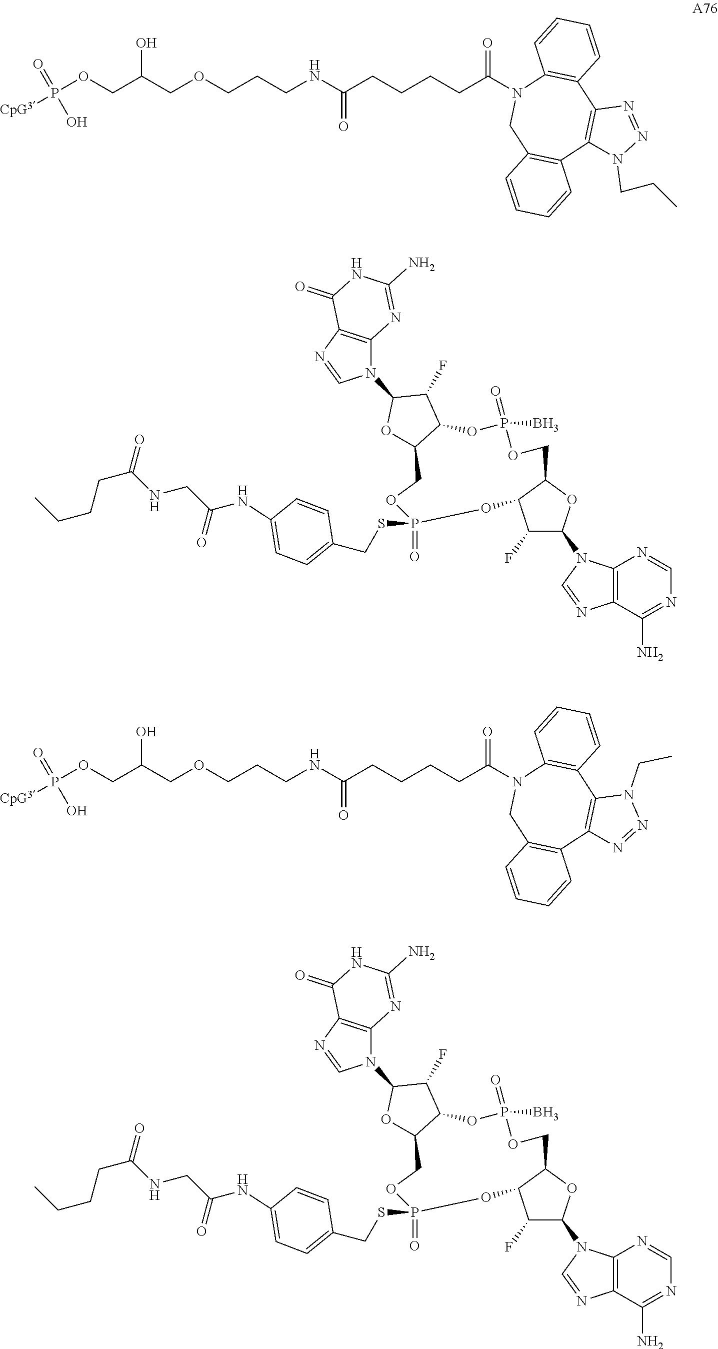

A mixture of regioisomers A76 was synthesized by the reaction of A18 with A4 according to the procedure described in Example A33. Clarity RP C18 3 um 4.6*100 mm, RT=14.25, 14.50 min. Calculated mass 11191 Da, measured mass 11191 Da.

### Example 77. Synthesis of A77

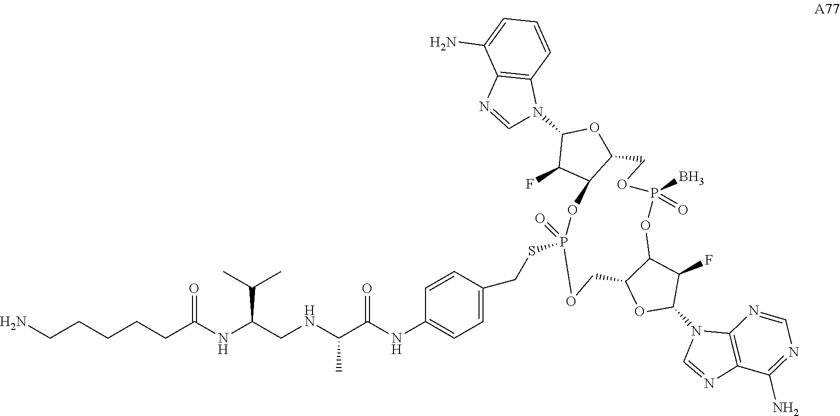

A77 was synthesized by the reaction of A5-I with I-15 according to the procedure described in Example A3. LC/MS [M−H]⁺=1089.4, ¹H NMR (400 MHz, DMSO-d₆) 8.50 (s, 1H), 8.19 (s, 1H), 8.15 (s, 1H), 7.96 (m, 1H), 7.45-7.43 (br d, J=8.0 Hz, 2H), 7.11-7.09 (br d, J=8.4 Hz, 2H), 6.46-6.22 (m, 2H), 6.02-5.74 (m, 1H), 5.65-5.36 (m, 3H), 4.54-4.38 (m, 2H), 4.37-4.23 (m, 3H), 4.12 (m, 2H), 3.89-3.73 (m, 3H), 3.26-3.23 (brt, J=6.4 Hz, 2H), 2.23-2.08 (m, 3H), 1.97-1.92 (br dd, J=6.4, 13.2 Hz, 1H), 1.58-1.39 (m, 4H), 1.27-1.25 (br d, J=7.2 Hz, 6H), 0.94-0.90 (brt, J=7.2 Hz, 1H), 0.85-0.80 (br dd, J=6.8, 13.6 Hz, 6H), 0.20 (s, 3H).

### Example 78. Synthesis of A78 (a Mixture of Regioisomers as Shown Below)

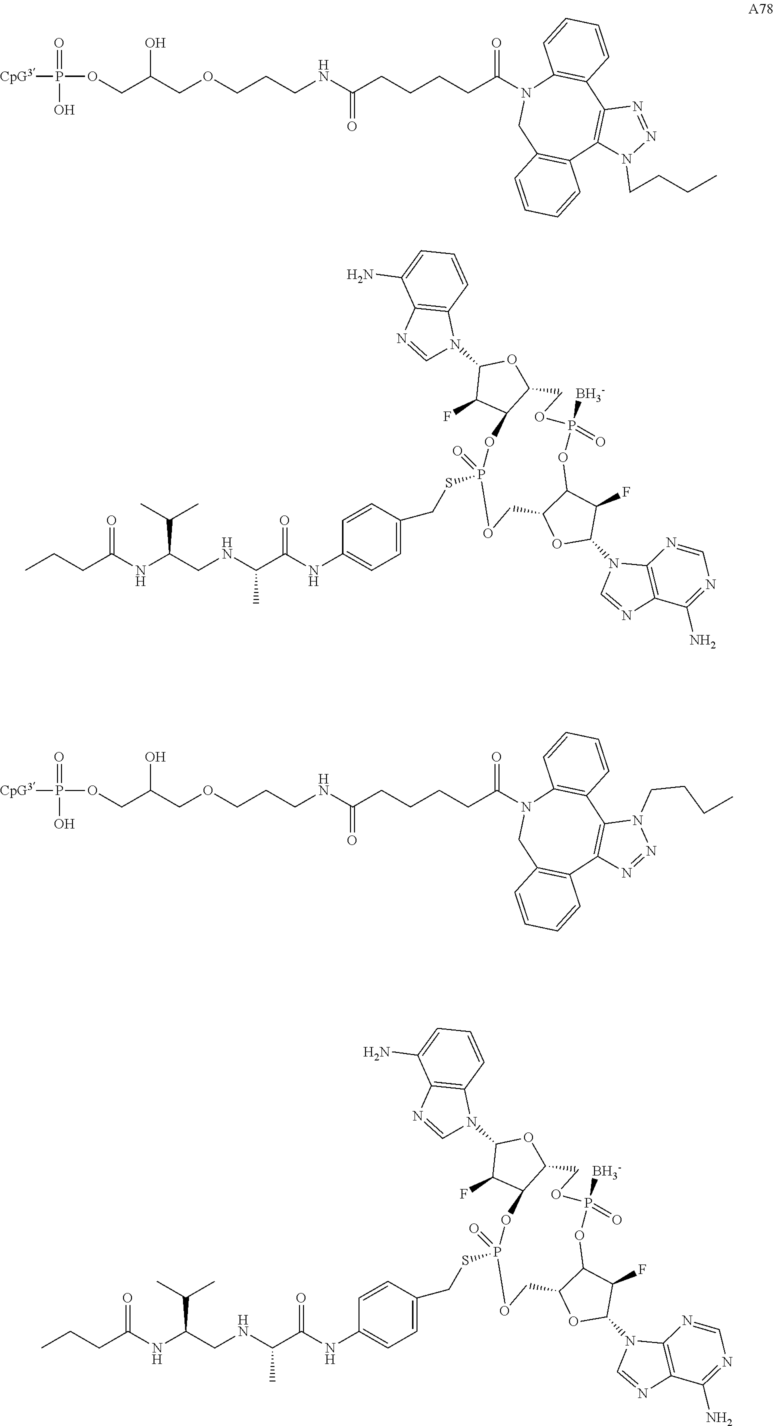

A mixture of regioisomers A78 was synthesized by the reaction of A77 with A18 according to the procedure described in Example A33. 19.0% yield. 95.0% purity. Clarity RP C18 3 um 4.6*100 mm, RT=12.95 min. Calculated mass 11288 Da, measured mass 11288.8 Da.

### Example 79. Synthesis of A79 (a Mixture of Regioisomers as Shown Below)

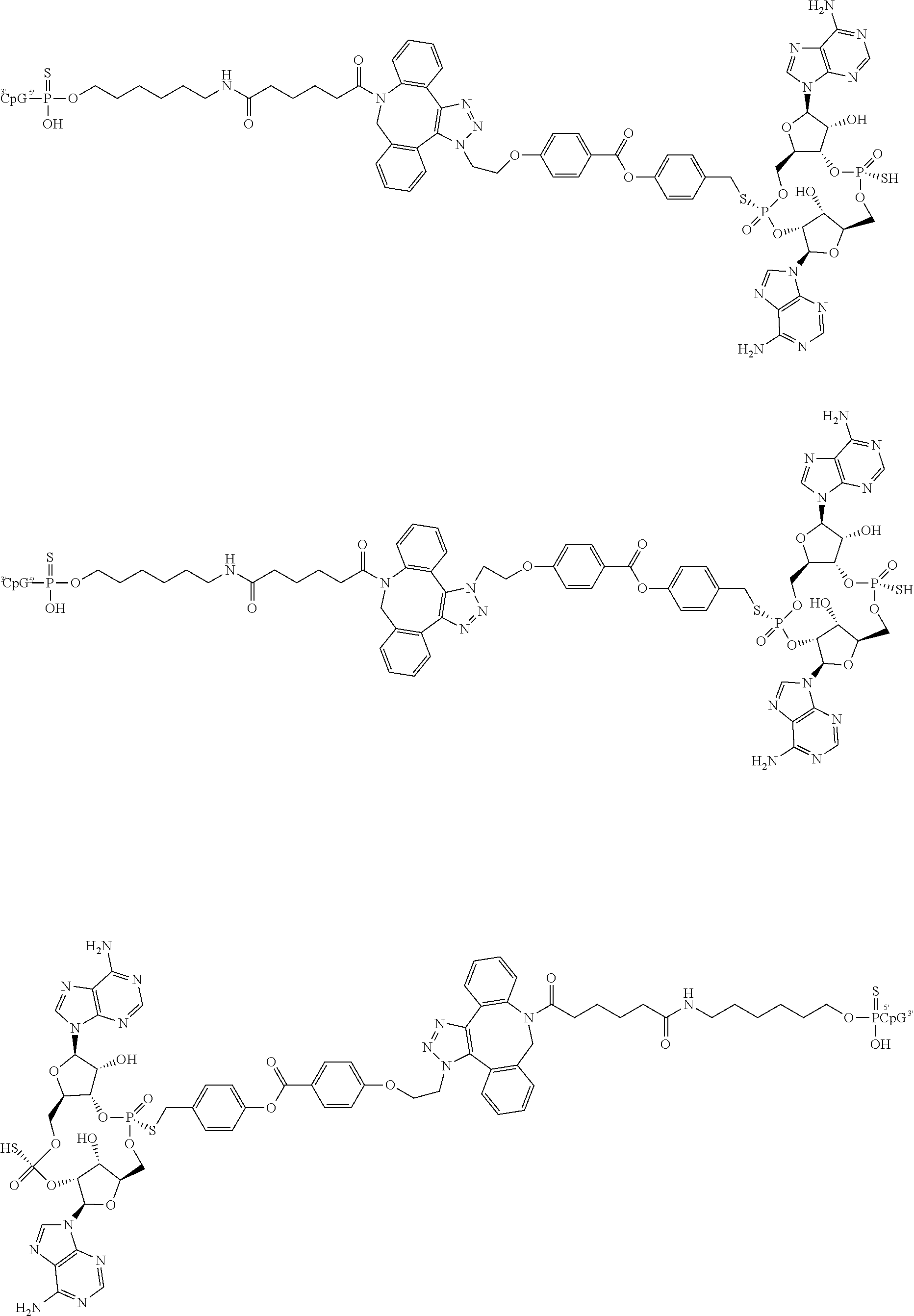

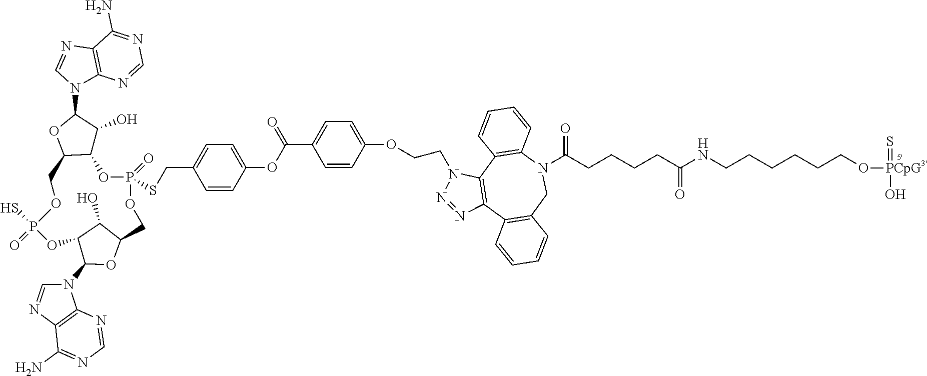

A mixture of regioisomers A79 is synthesized by the reaction of A67 with A51-I and A51-II according to the procedure described in Example A33.

### Example 80. Synthesis of A80 (a Mixture of Regioisomers as Shown Below)

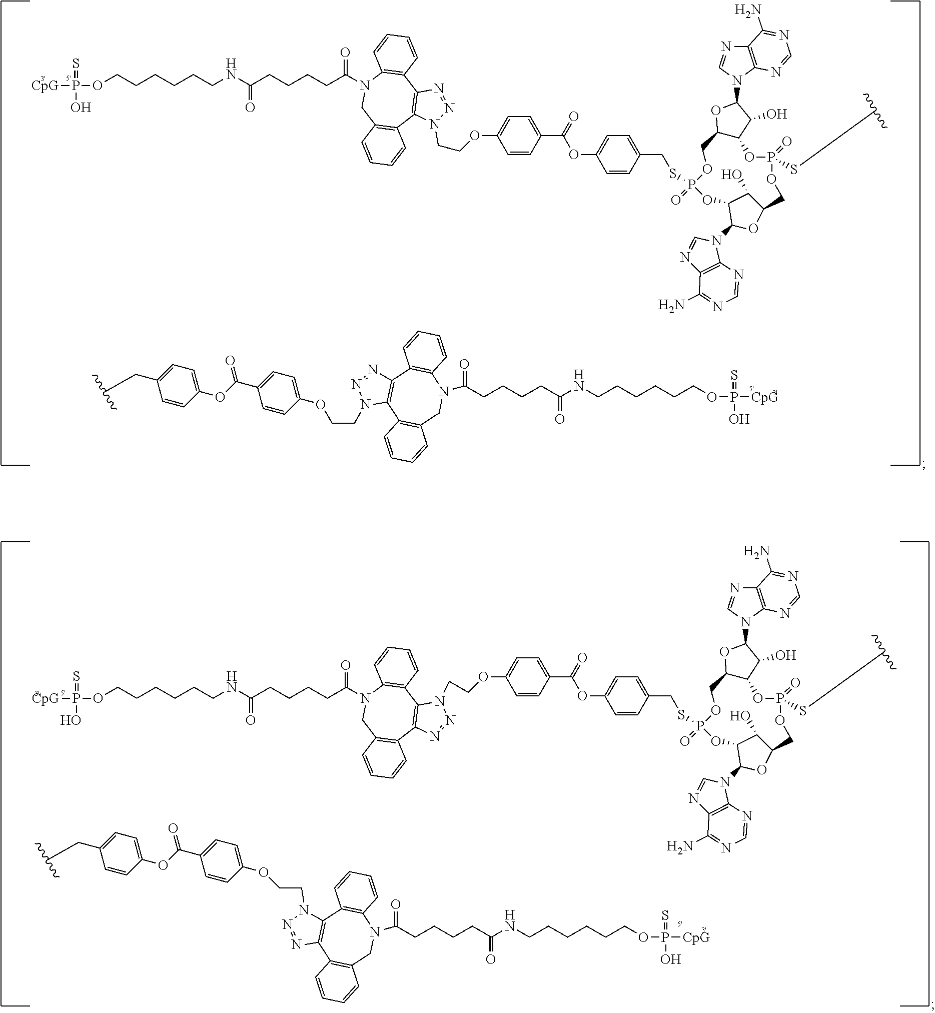

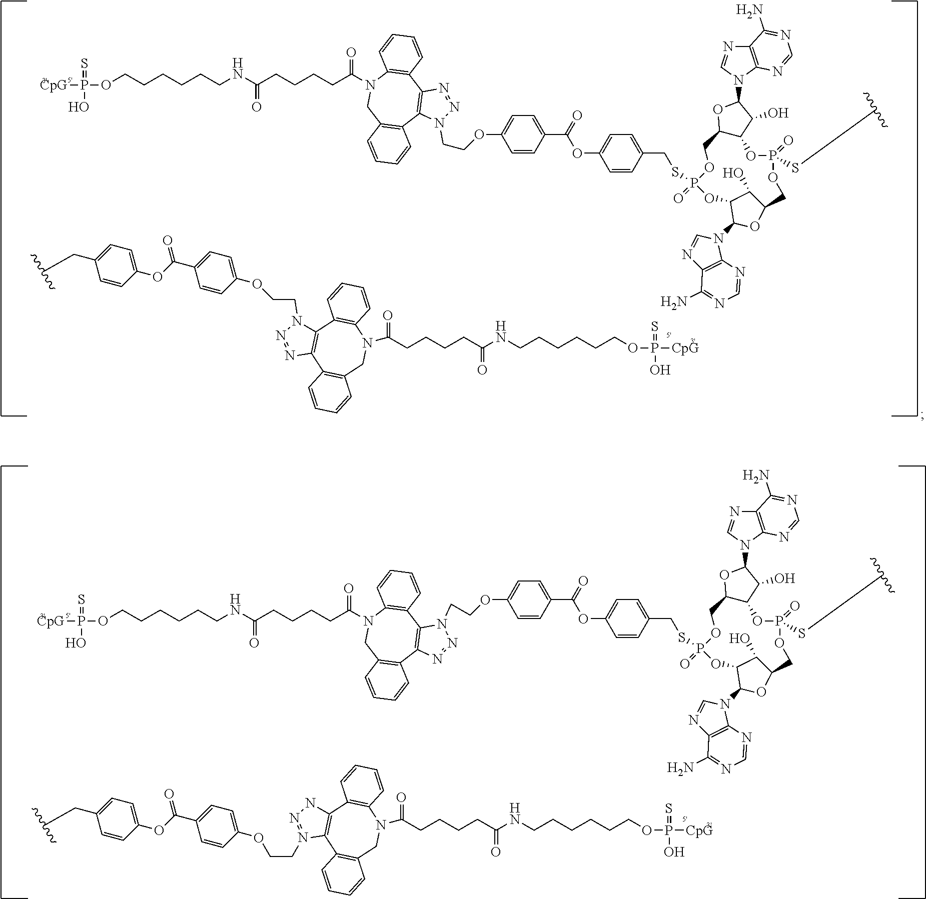

A mixture of regioisomers A80 is synthesized by the reaction of A67 with A52 according to the procedure described in Example A33.

### Example 81. Synthesis of A81 (a Mixture of Regioisomers as Shown Below)

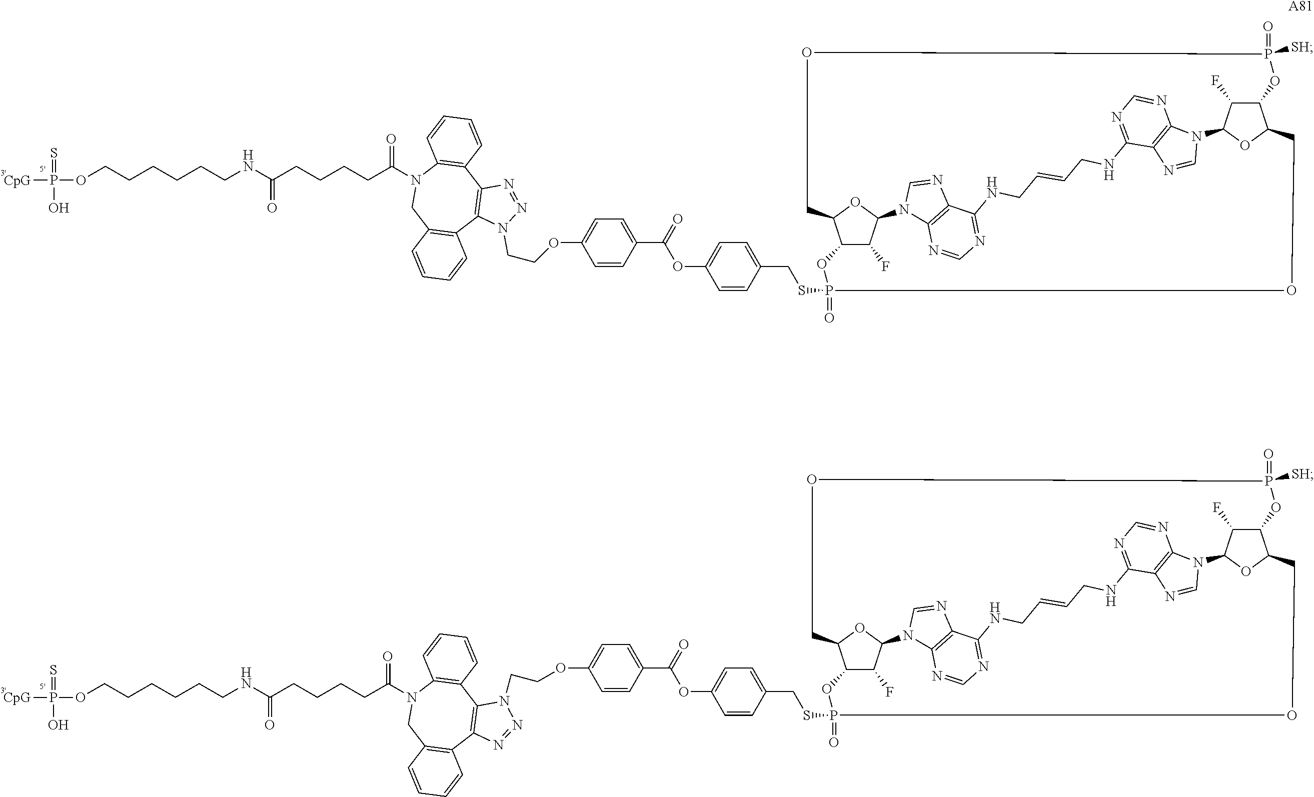

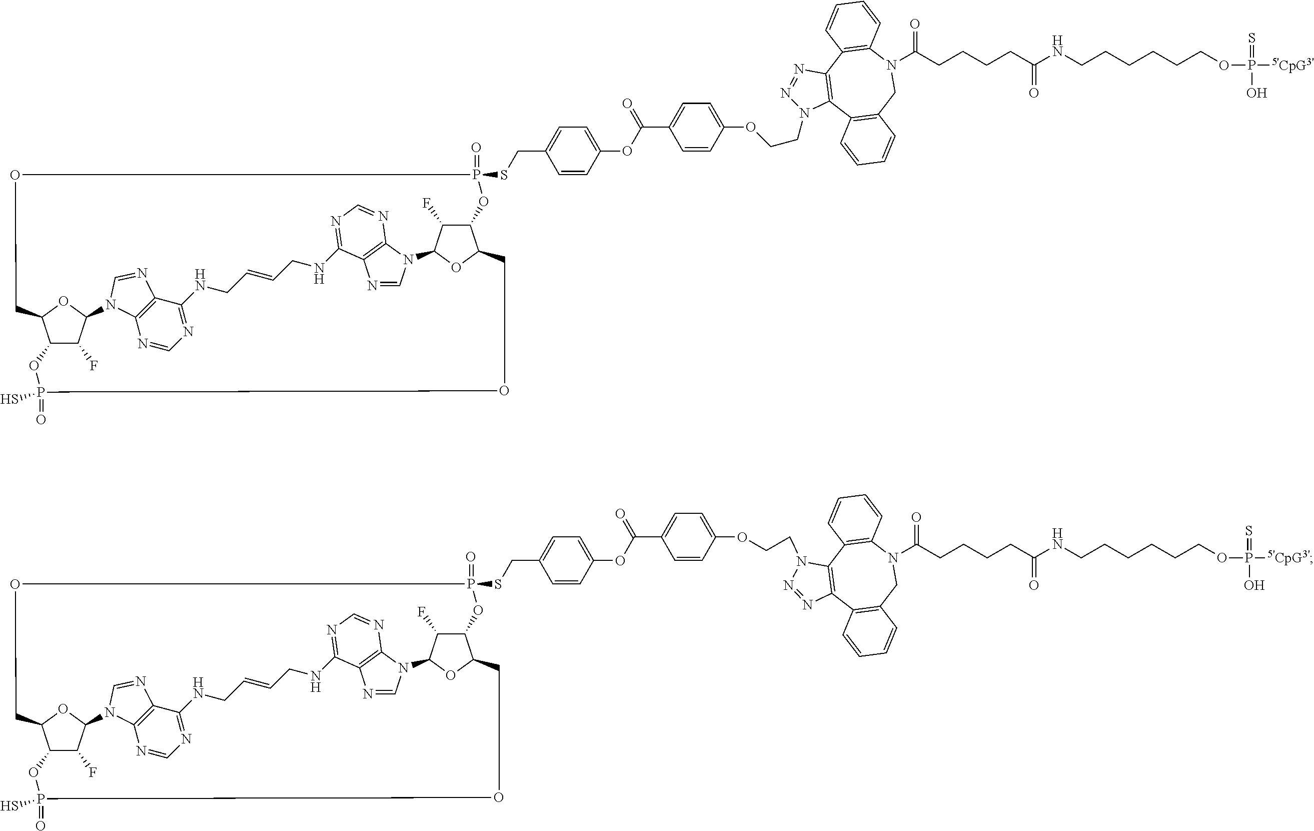

A mixture of regioisomers A81 is synthesized by the reaction of A67 with A43-I and A43-II according to the procedure described in Example A33.

### Example 82. Synthesis of A82 (a Mixture of Regioisomers as Shown Below)

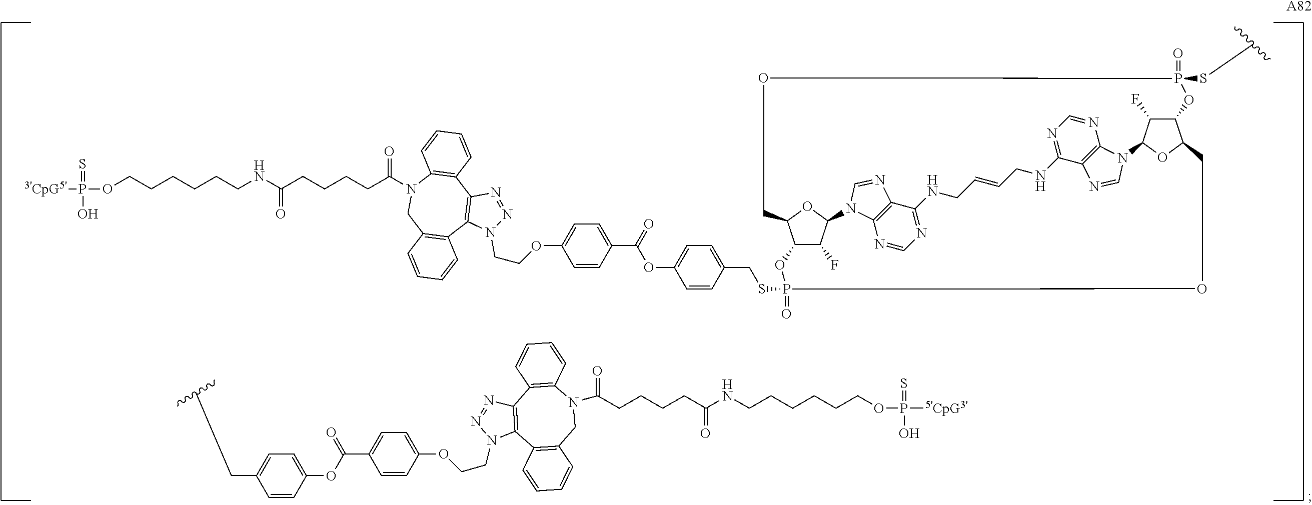

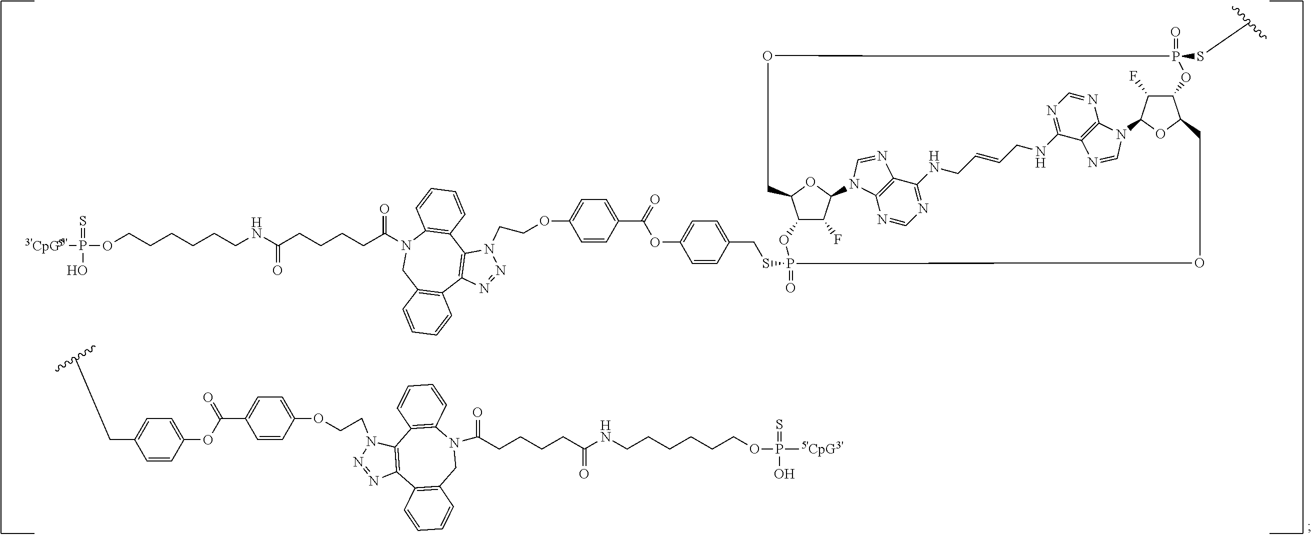

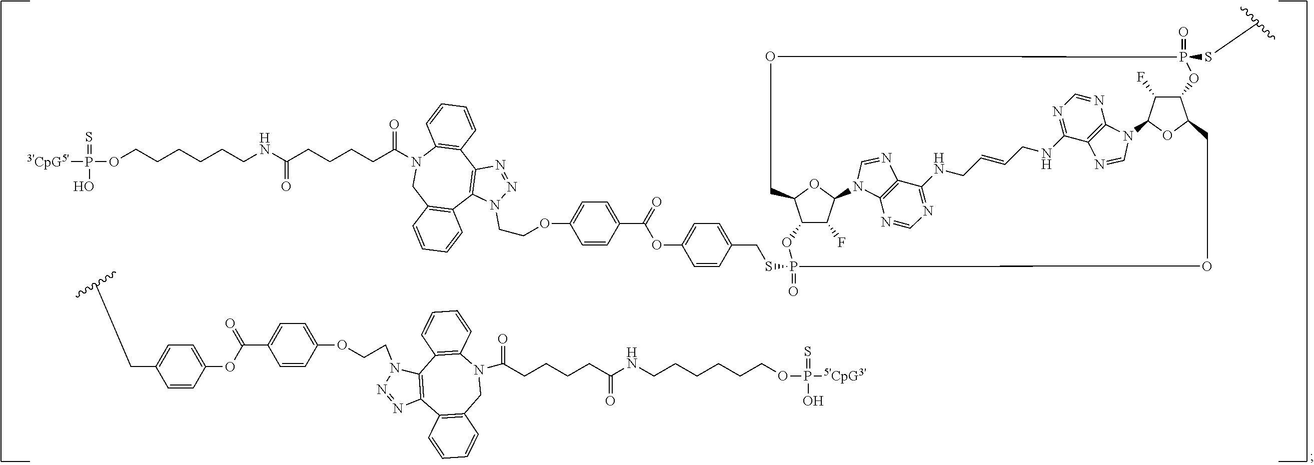

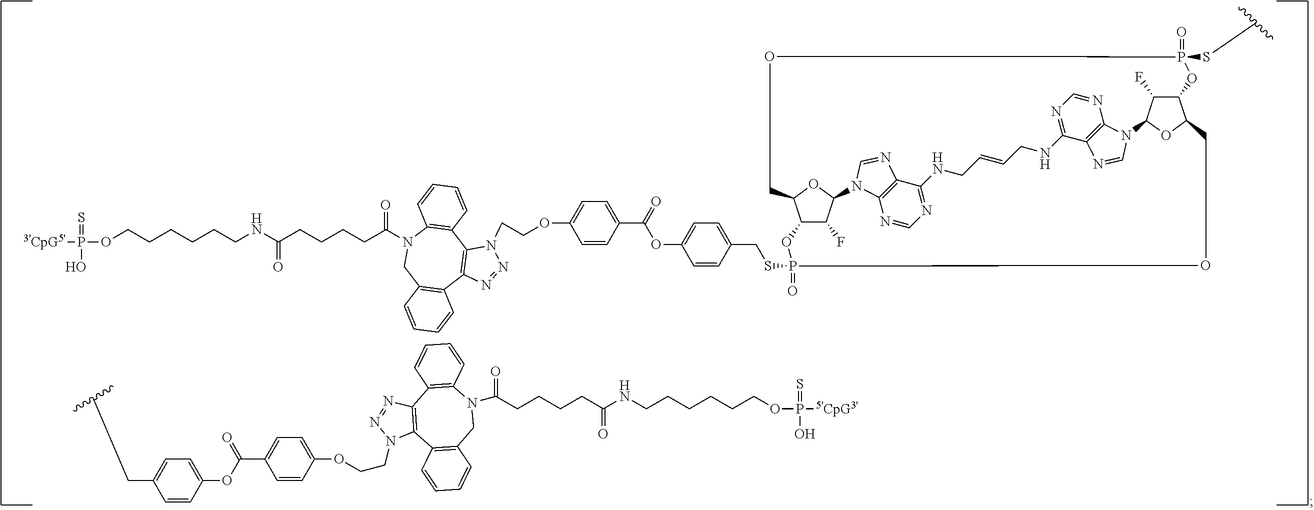

A mixture of regioisomers A82 is synthesized by the reaction of A67 with A46 according to the procedure described in Example A33.

### Example 83. Synthesis of A83 (a Mixture of Regioisomers as Shown Below)

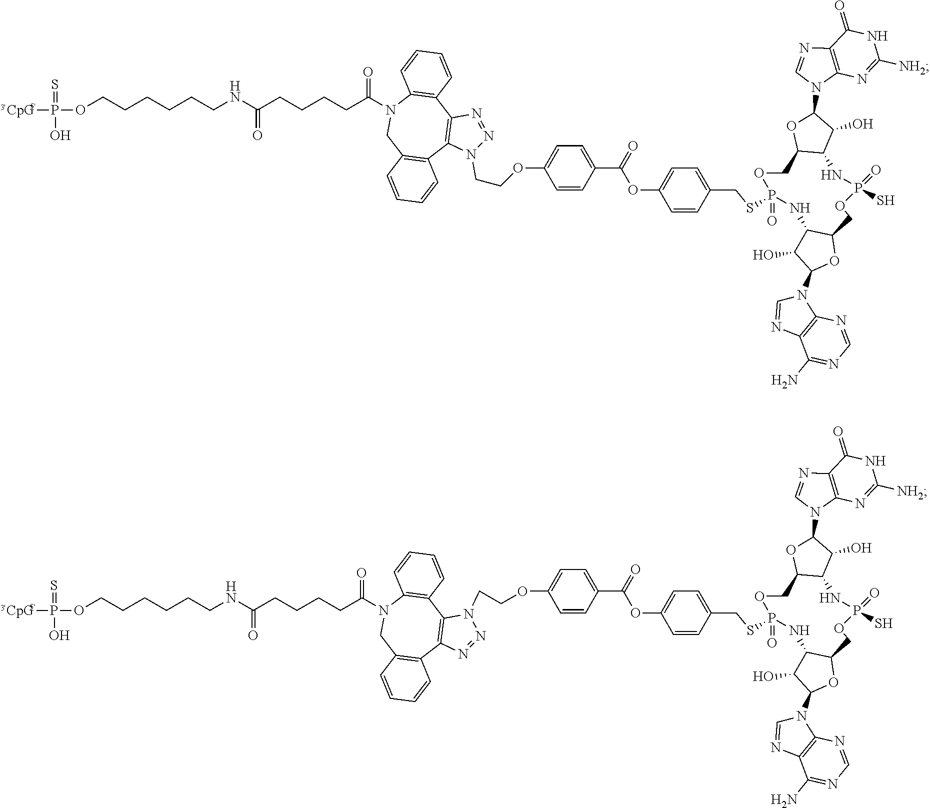

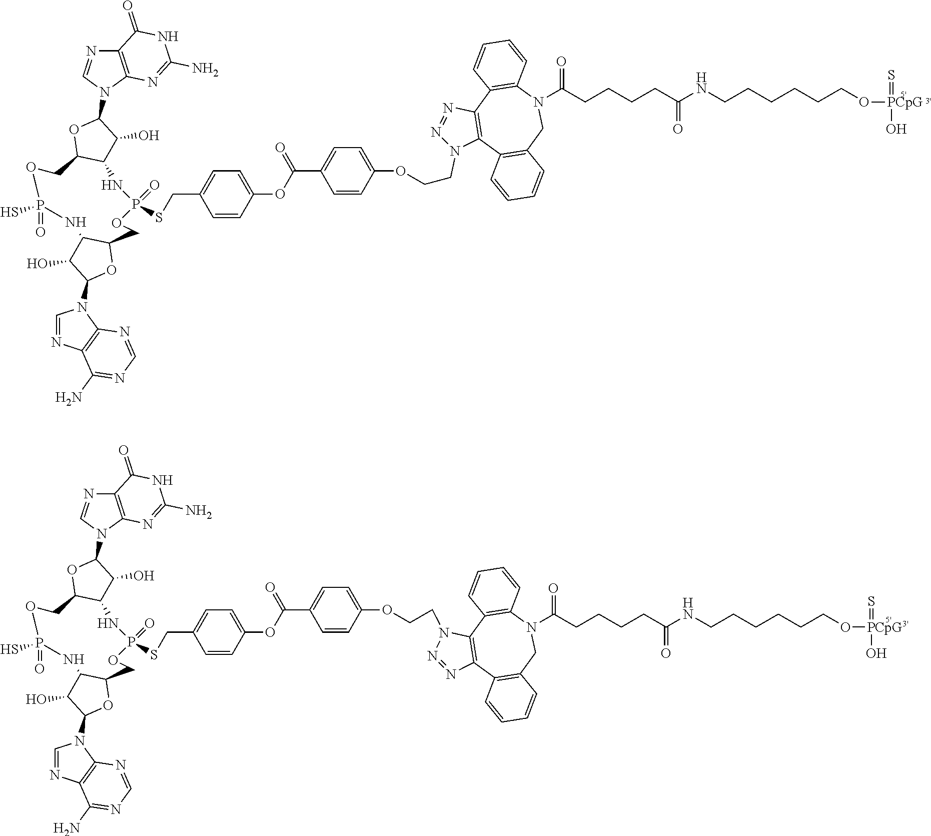

A mixture of regioisomers A83 is synthesized by the reaction of A67 with A59-I and A59-II according to the procedure described in Example A33.

### Example 84. Synthesis of A84 (a Mixture of Regioisomers as Shown Below)

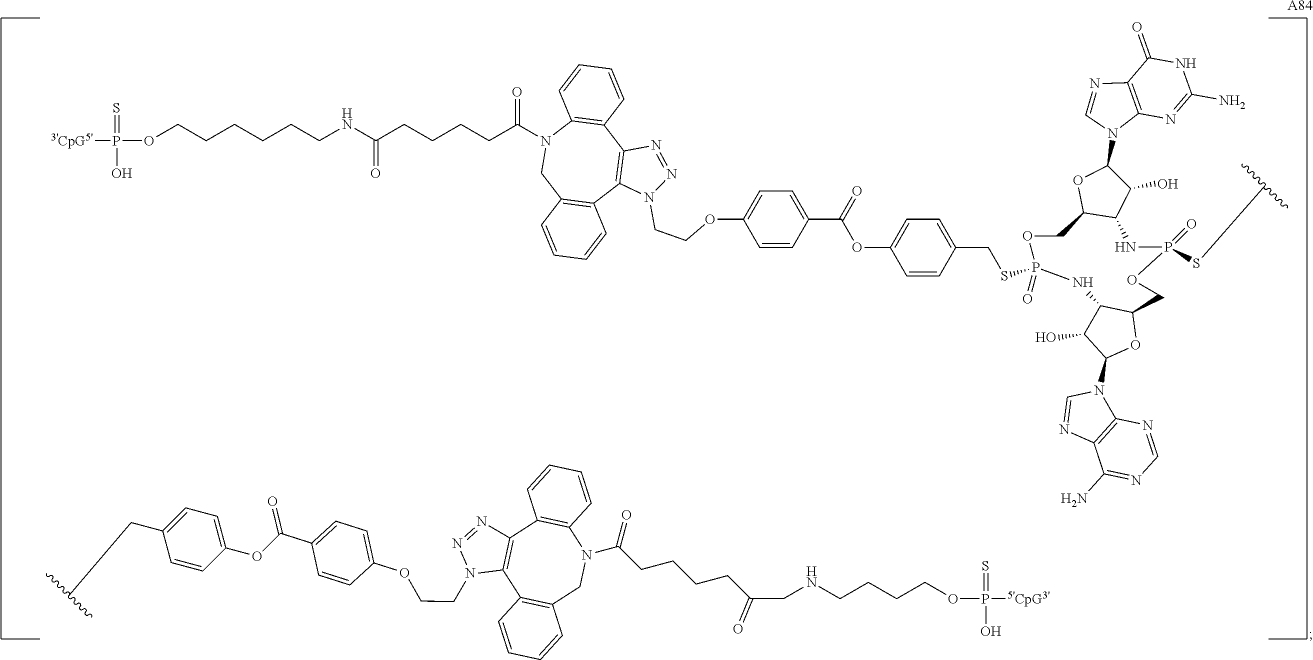

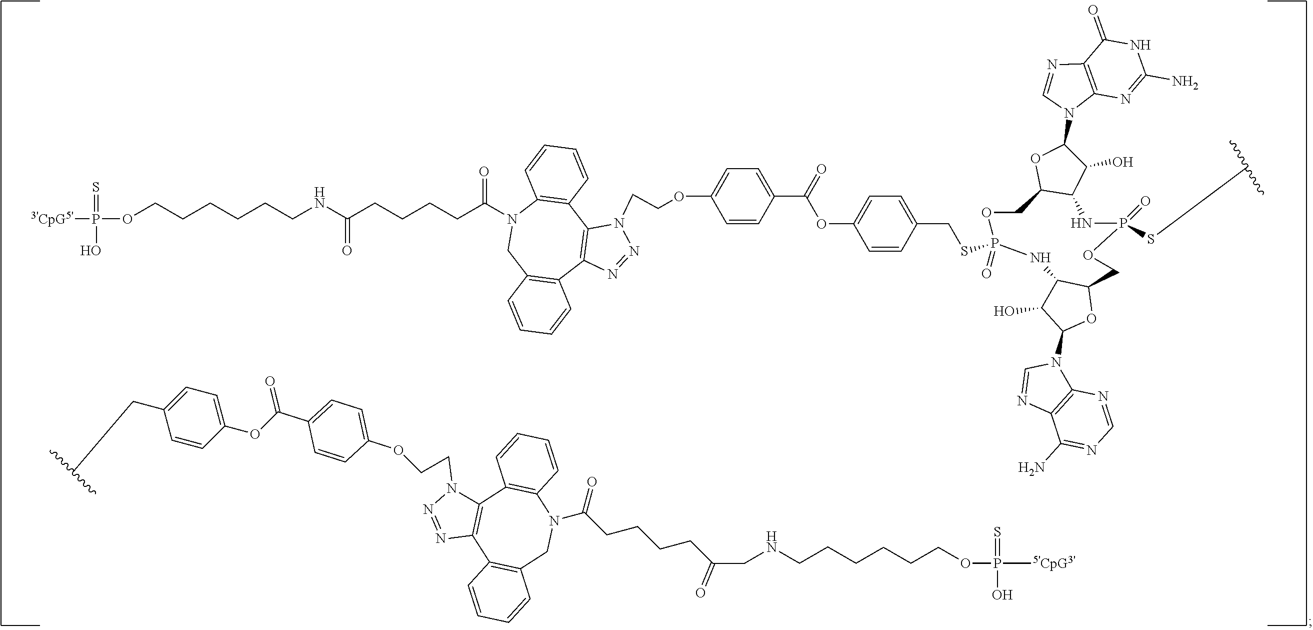

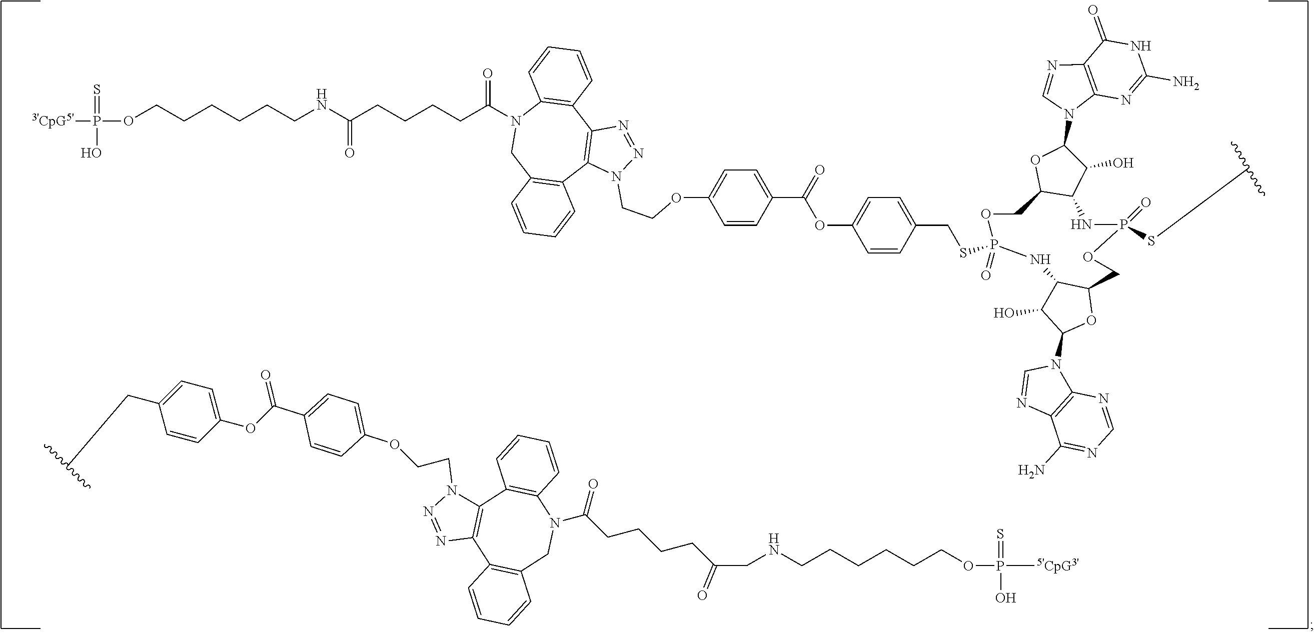

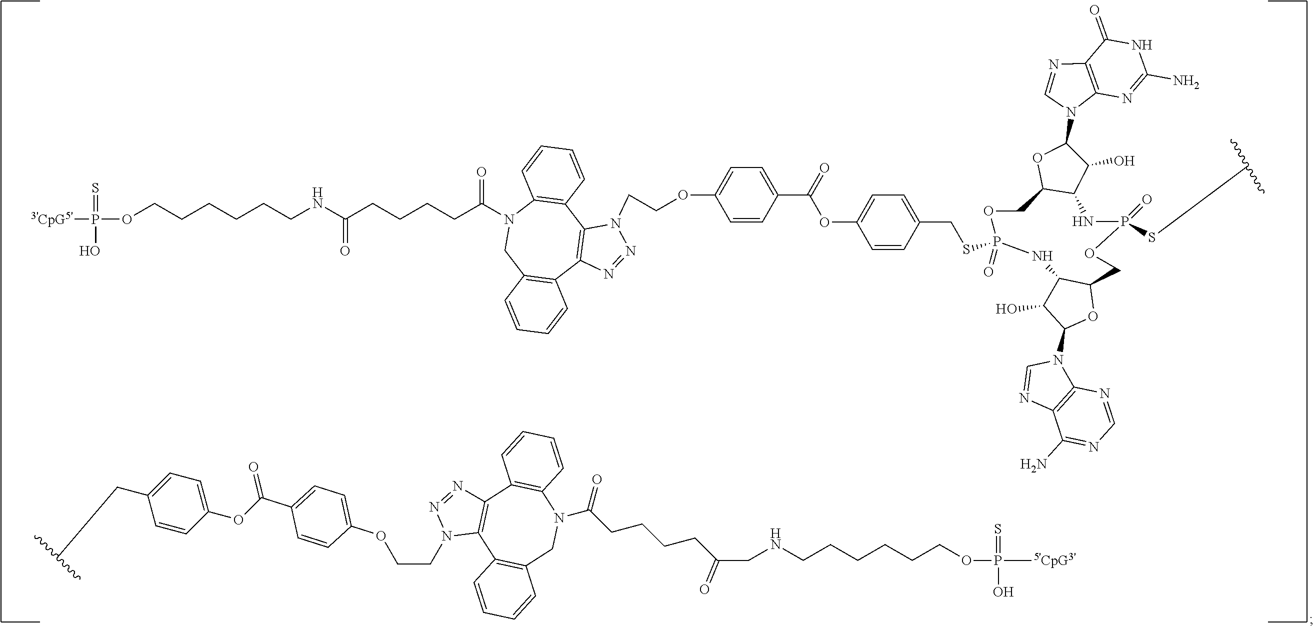

A mixture of regioisomers A84 is synthesized by the reaction of A67 with A60 according to the procedure described in Example A33.

### Example 85. Synthesis of A85 (a Mixture of Regioisomers as Shown Below)

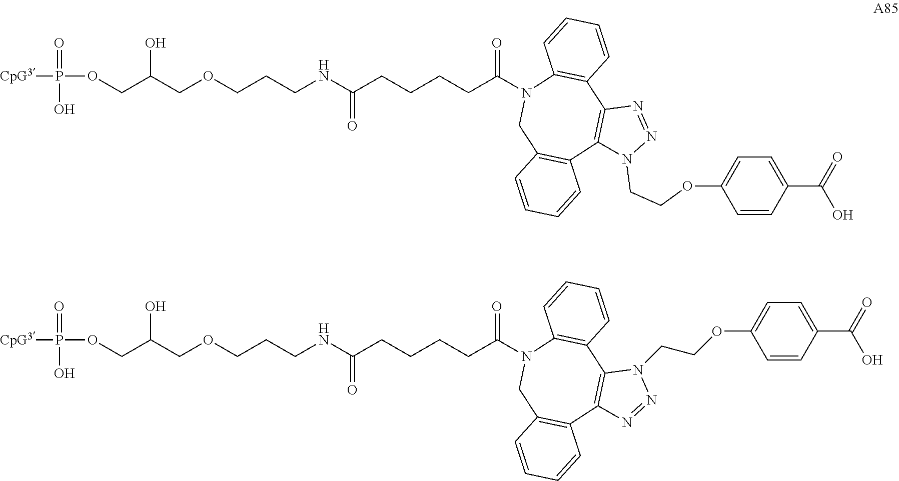

4-(2-Azidoethoxy)benzoic acid (1.63 mg, 7.84 umol, 2.00 eq) in DMF (0.1 mL) was added to a solution of A18 (40.0 mg, 3.92 umol, 1.00 eq) in PBS (0.1 mL, pH=7.4) at 26° C. and the reaction was stirred for 2 hrs, then the reaction solution was purified by prep-HPLC (neutral condition, column: Clarity RP 21.2*150 mm; mobile phase: [0.1M TEAA-ACN]; B %: 5%-35%, 10 min) to give A85 (7.50 mg, 18.7% yield) as a solid. RP C18 3 um 4.6*100 mm, RT=9.98 min; purity: 98.8%; LC/MS calculated mass 10405 Da, measured mass 10405 Da.

### Example 86. Synthesis of A86 (a Mixture of Regioisomers as Shown Below)

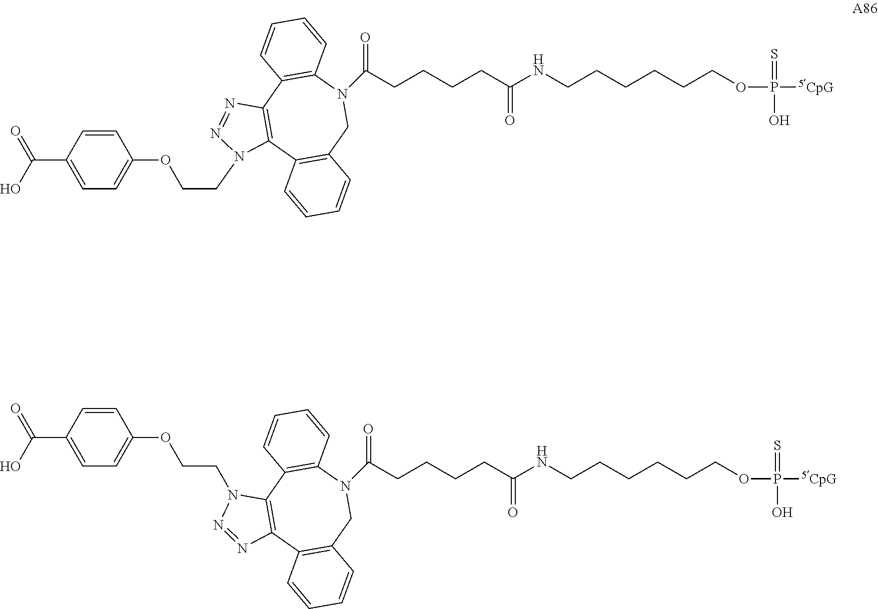

A86 was synthesized by the reaction of A67 and 4-(2-azidoethoxy)benzoic acid according to the procedure described in Example A33. LC/MS calculated mass 10389 Da, measured mass 10388.8 Da.

### Example 87. Synthesis of A87 (a Mixture of Regioisomers as Shown Below)

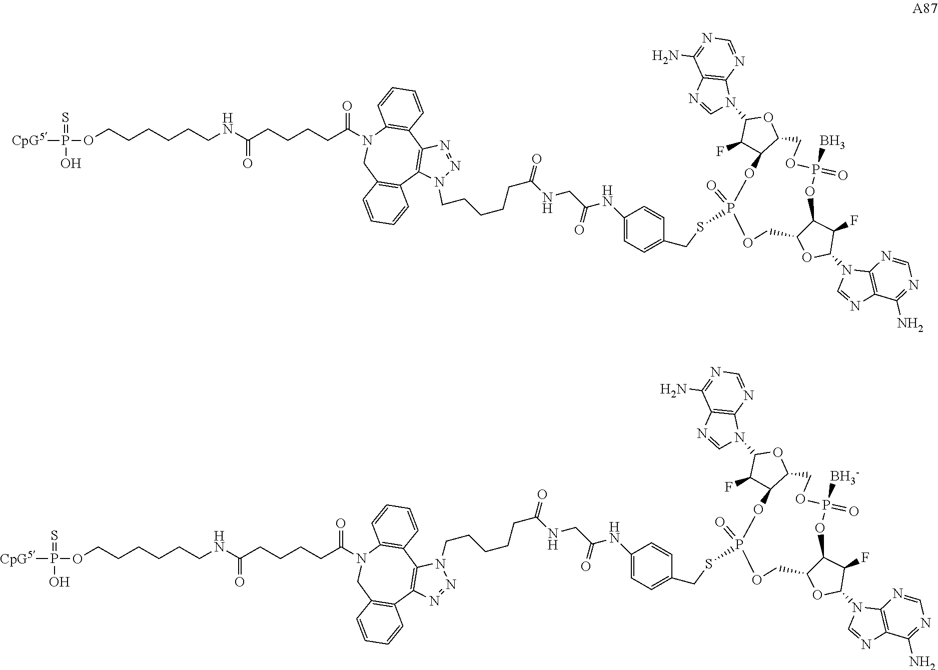

A87 was synthesized by the reaction of A67 and A8 according to the procedure described in Example A33. Purity: 88%, LC/MS calculated mass 11159 Da, measured mass 11159.6 Da.

### Example 88. Synthesis of A88

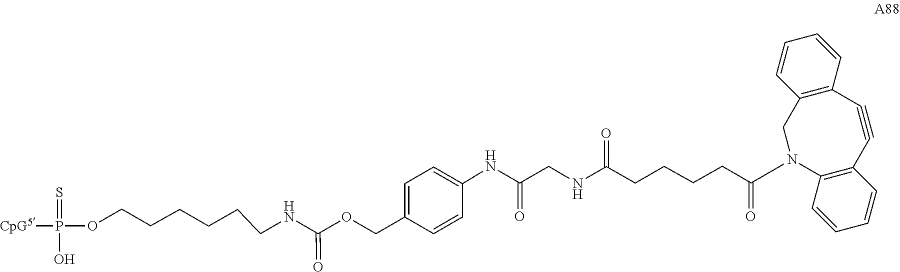

A88 was synthesized by the reaction of A72-a and A12 according to the procedure described in Example A18. LC/MS Calculated mass 10388 Da, measured mass 10388 Da.

### Example 89. Synthesis of A89 (a Mixture of Regioisomers as Shown Below)

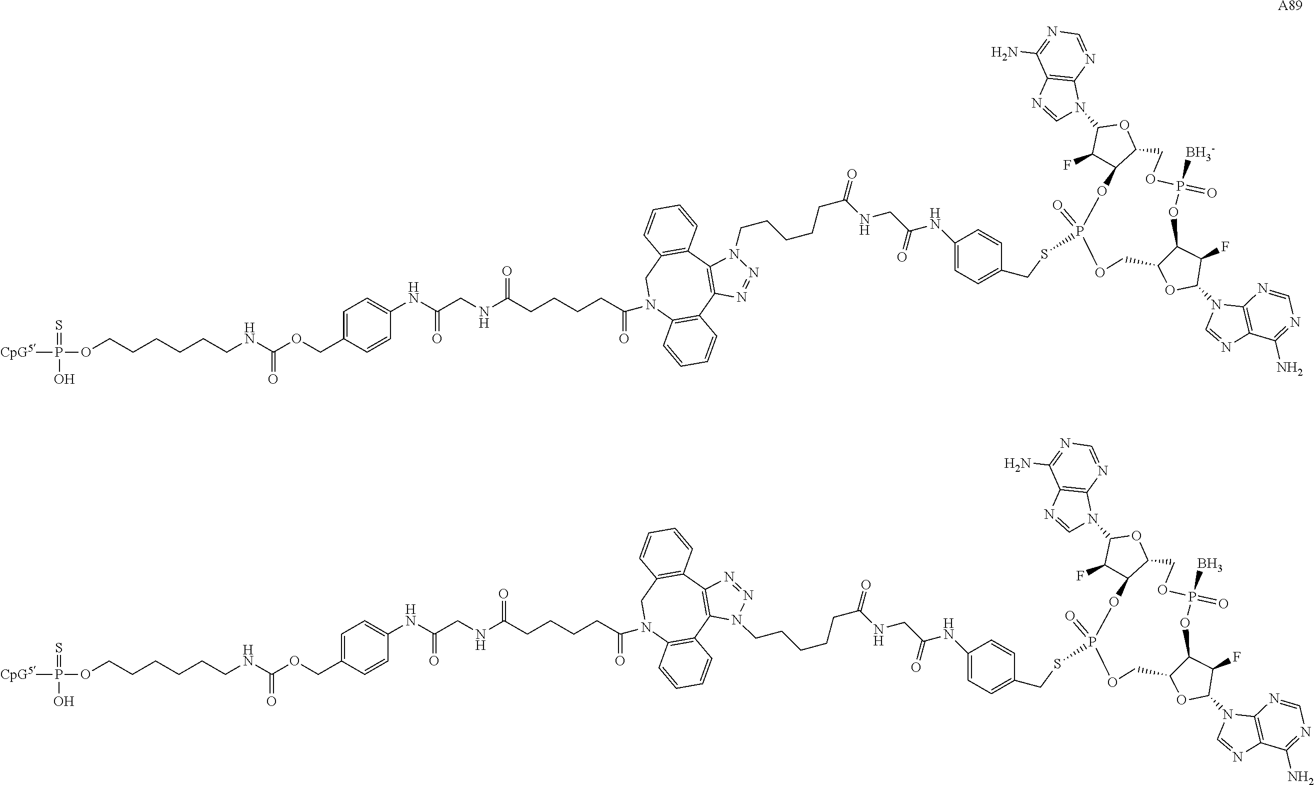

A89 was synthesized by the reaction of A88 and A8 according to the procedure described in Example A33. LC/MS calculated mass 11365 Da, measured mass 11367 Da.

### Example 90. Synthesis of A90 (a Mixture of Regioisomers as Shown Below)

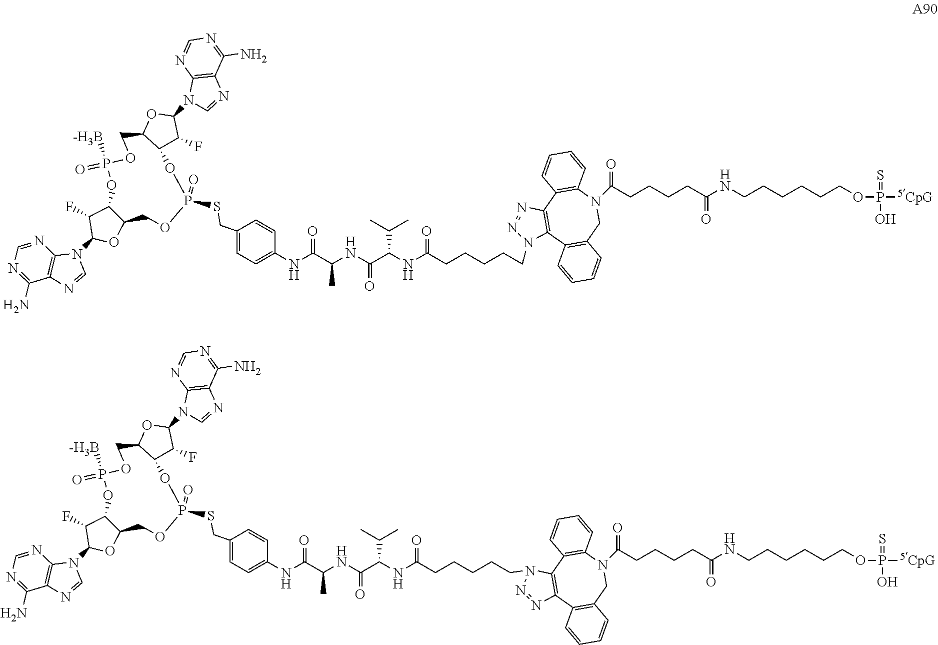

A90 was synthesized by the reaction of A67 and A77 according to the procedure described in Example A33. Purity 90.3%, LC/MS calculated mass 11272 Da, measured mass 11272.6 Da.

### Biological Assays

### Example 91. Activation of Human STING Signaling in THP1 Cell Line

THP1-Dual™ KI-hSTING-R232 cells (obtained from InvivoGen) are maintained in RPMI1640 medium with 100 μg/ml of Zeocin and 10 μg/ml of blasticidin at 37° C., 5% CO₂. To set up assay, 0.5×10⁶ cells/ml, 180 μl/well of THP1-Dual™ KI-hSTING-R232 cells were seeded in 96-well plate. The cells were treated with 20 μl/well of compounds at 8 concentrations in duplicate in 96-well plate, and incubate for 24 h. To measure IRF promoter activity, 15 μl of cell culture supernatant was mixed with 45 μl QUANTI-Luc in an opaque 96-well plate and immediately measured using Luminometer; to measure NF-κB promoter activity, 15 μl of supernatant was mixed with 90 μl QUANTI-Blue, and incubated at 37° C. for 90 minutes, and read at OD650 nm. EC₅₀ was calculated by nonlinear regression fit with 4-parameter responsive curve fitting using GraphPad Version 8 software (Table 1).

TABLE 1
EC₅₀ in THP1 cell line

| | |
|---|---|
| Compound ID | EC₅₀ IRF3 (μM) |

Assay Results 1 (average of more than 2 runs)

| | |
|---|---|
| A1 | 0.45 |
| A2 | 0.054 |
| A3 | 0.002 |
| A5-I | 0.22 |
| A5-II | 8.4 |
| A6-I | 0.20 |
| A6-II | 0.89 |

Assay Results 2 (single run)

| | |
|---|---|
| A1 | 0.564 |
| A5-I | 0.522 |
| A17 | 0.022 |
| A19 | 0.058 |
| A33 | 0.054 |
| A34 | 0.266 |
| A38 | 0.054 |
| A68 | 0.114 |
| A69 | 0.184 |
| A71 | 1.223 |
| A73 | 1.563 |
| A74 | 3.994 |
| A75 | 0.053 |

### Example 92. Evaluation of Compounds in 293T-Dual hSTING R232 Cells

293T-Dual hSTING 8232 cells were cultured in DMEM medium supplemented with 10% heat inactivated FBS, 2 mM L-glutamine, 4.5 g/L glucose, Pen-Strep (100 U/mL-100 μg/mL), 100 μg/mL Normocin, 10 μg/mL blasticidin, 100 μg/mL hygromycin B Gold and 100 μg/mL zeocin (complete medium). 180 uL of 293T-Dual hSTING 8232 cells (InvivoGen) at 0.25×10⁶ cells/ml were seeded in a well of a 96-well plate, and cultured at 37° C. incubator for 2 hr. A test compound with a serial of 1:3 dilution was prepared in complete medium, and 20 μL of the compound was added to the corresponding well, and incubated with the cells at 37° C. incubator for 24 hr.

To determine the effect on interferon regulatory factor (IRF) activation, 15 μL of cell culture supernatant was transferred into a fresh 96-half area-well plate, 90 μL of Quanti-Blue was added to each well and incubated at 37° C. for 90 min. OD at 650 nm was recorded. EC₅₀ was calculated by nonlinear regression fit with 4-parameter responsive curve fitting using GraphPad Version 8 software (Table 2).

TABLE 2
EC₅₀ in 293T-Dual hSTING R232 cells

| Compound ID | EC₅₀ IRF3 (μM) |
|---|---|
| A1 | 0.059 |
| A2 | 0.070 |
| A3 | 0.022 |
| A5-I | 0.024 |
| A6-I | 0.057 |
| A6-II | 0.35 |
| A17 | 0.451 |
| A19 | 0.414 |
| A33 | 0.112 |
| A34 | 1.571 |
| A38 | 0.177 |
| A69 | 0.440 |
| A71 | 1.889 |

### Example 93. Evaluation of Compounds A5-I, A5-II, A6-I and A6-II in MC38 Murine Syngeneic Model

To Evaluate the in vivo efficacy of A5-I, A5-II, A6-I and A6-II, C57BL/6 mouse was subcutaneously inoculated in the right flank with MC38 tumor cells (1×10⁶) in 40 ul of HBSS for tumor development. Treatment started when tumor size reached around 80-100 mm³, on day 7 after tumor cell implantation. Compounds were administered by IT injection and repeated every three days with total three injections. Compounds A5-I, A5-II, A6-I and A6-II were dosed at 5 ug/mouse. Tumor volumes were measured twice to thrice weekly. On 66 days after tumor cell implantation, tumor-free mice and 3 naive mice were subcutaneously inoculated in the left flank with MC38 tumor cells (1×10⁶) in 0.1 ml of HBSS for tumor development.

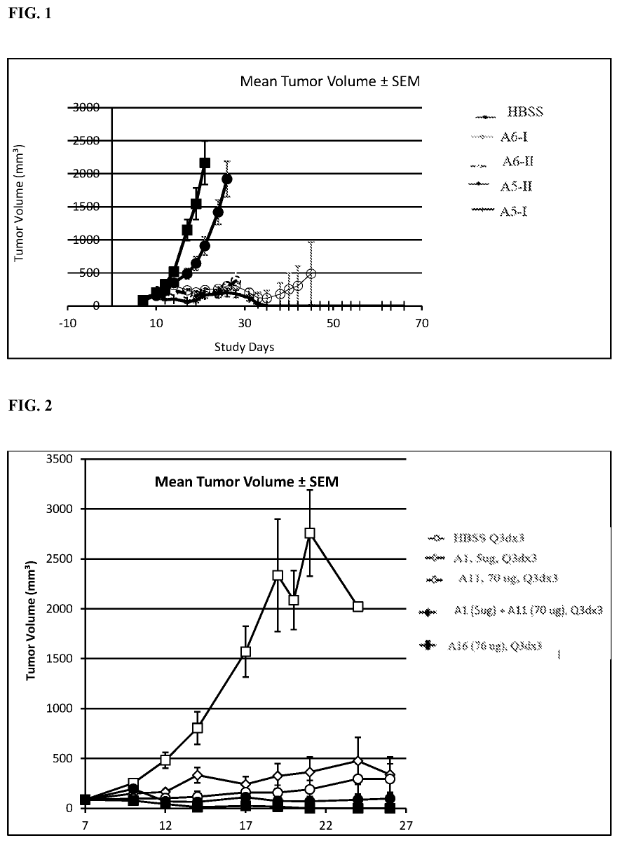

As shown in FIG. 1, compounds A5-I, A6-I and A6-II as a single agent demonstrated significant anti-tumor efficacy at 5 ug/mouse dosing in this subcutaneous MC38 murine colon cancer model. Treatment of compound A5-I at 5 ug/mouse dosing resulted in complete regression for all treated mice. The cured mice from the treatment groups were resistant to the re-challenged MC38 tumor cell (not shown here), indicating the tumor-free mice developed long-term memory to tumor antigens expressed in MC38 tumor.

### Example 94. Evaluation of Compound A1, A11 and Conjugate A16 in MC38 Murine Syngeneic Model

To evaluate the in vivo efficacy of A1, A11 and A16, C57BL/6 mouse was subcutaneously inoculated in the right flank with MC38 tumor cells (1×10⁶) in 40 ul of HBSS for tumor development. Treatment started when tumor size reached around 80-100 mm³, on day 7 after tumor cell implantation. A1, A11 and A16 were administered by IT injection. A1 was dosed at 5 ug/mouse, A11 was dosed at 70 ug/mouse and A16 was dosed at 76 ug/mouse respectively and repeated every three days with total three injections. Tumor volumes were measured twice to thrice weekly.

As shown in FIG. 2, STING-TLR9 conjugate A16 showed better anti-tumor activity compared to the mixture of STING agonist A1 and TLR9 agonists A11, resulting in complete tumor regression for all treated mice. Combination of STING and TLR9 agonists demonstrated significantly improved anti-tumor activity compared to the treatment of either STING agonist A1 or TLR9 agonist A11 alone. No significant body weight loss was observed for all treated mice.

### Example 95. Evaluation of Compounds in PBMC Assay

Stimulation of human peripheral blood mononuclear cells to secrete interferon-a. Frozen human peripheral blood mononuclear cells (PBMC) was thawed and cultured in RPMI 1640 supplemented with 2 mM L-glutamine, 4.5 g/L glucose, 10% heat-inactivated fetal bovine serum, and 1% Penicillin/Streptomycin (10,000 U/mL, Thermo Fisher Scientific). 0.3×10 6 cells/well of PBMC were seeded in 96-well plate. The cells were treated with compounds, for example A11, starting at 2 mM with 1:3 serial dilution of 8 concentrations in 96-well plate in duplicate. After 24 h incubation, the Interferon-α (IFNα) level in the supernatant from each well was detected with anti-IFNα ELISA kit (MabTech Cat #: 3425-1H-6. Human IFN-α pan ELISA development kit (HRP)), according to the manufacturer's instruction. Briefly, high protein binding 96-well ELISA plate (Costar, Cat #3361) was coated with 100 ul/well of mAbs MT1/3/5 at 4 μg/mL, diluted in phosphate buffered saline (PBS), pH7.4. The plate was incubated overnight at 4-8° C., washed twice with PBS, blocked with 200 mL/well of 0.1% bovine serum albumin (BSA) in PBS containing 0.05% Tween20, and incubated at room temperature for 1 hour. After the plate was washed with PBS containing 0.05% of Tween20 for 5 times, 100 mL of supernatant from each TLR9 agonist compound treated well were transfer to MT1/3/5 Ab coated ELISA plate and incubated for 2 h on a rotatory shaker (Microplate shaker, Thermo Scientific) at 400 rpm, at room temperature. After the plate was washed 5 times with PBS containing 0.05% Tween-20, 100 mL/well of MT2/4/6-biotin at 1 mg/mL diluted in ELISA diluent buffer were added into ELISA plate. The plate was mixed on the rotatory shaker at 400 rpm for 1 h followed by washing with PBS containing 0.05% Tween20 for 5 times. The plate was added with 100 mL/well of Streptavidin-HRP diluted 1:1000 in incubation buffer, incubated for 1 h at room temperature on the shaker. After the plate was washed with PBS containing 0.05% Tween20 for 5 times, 100 ml/well of TMB substrate (Mabtech product code 3652-F10) was added on plate. The reaction was stopped by adding 100 mL/well of stop solution. The optical density of each well was read on an ELISA reader at OD450 nm.

The EC₅₀ for each compound was calculated with nonlinear regression fit of 4 parameter logistic equation using GraphPad Version 8 software. The data of EC₅₀ was summarized in Table 3.

TABLE 3
EC50 of IFN-α induction in PBMC

| Compound ID | EC₅₀ of IFNα (μM) |
|---|---|
| A11 | 0.24 |
| A12 | 0.21 |
| A13 | 0.23 |
| A14 | 0.22 |
| A15 | 0.09 |
| A16 | 0.04 |
| A17 | 0.04 |
| A23 | 0.12 |
| A24 | 0.13 |
| A25 | 0.10 |
| A26 | 0.06 |
| A19 | 0.042 |
| A34 | 0.080 |
| A33 | 0.035 |
| A68 | 0.082 |
| A38 | 0.073 |
| A69 | 0.079 |
| A71 | 0.040 |
| A73 | 0.073 |
| A74 | 0.027 |
| A75 | 0.028 |
| A78 | 0.049 |
| A86 | 0.048 |
| A87 | 0.167 |
| A89 | 0.161 |
| A90 | 0.135 |

### Example 96. Evaluation of Compound A11, A12, A13, A26 in MC38 Murine Syngeneic Model

To evaluate the in vivo efficacy of compounds A11, A12, A13, and A26, C57BL/6 mouse was subcutaneously inoculated in the right flank with MC38 tumor cells (1×10⁶) in 40 ul of HBSS for tumor development. Treatment started when tumor size reached around 80-100 mm³, on day 7 after tumor cell implantation. All compounds A11, A12, A13, and A26 were administered by IT injection at 70 ug/mouse and repeated every three days with total six injections. Tumor volumes were measured twice to thrice weekly.

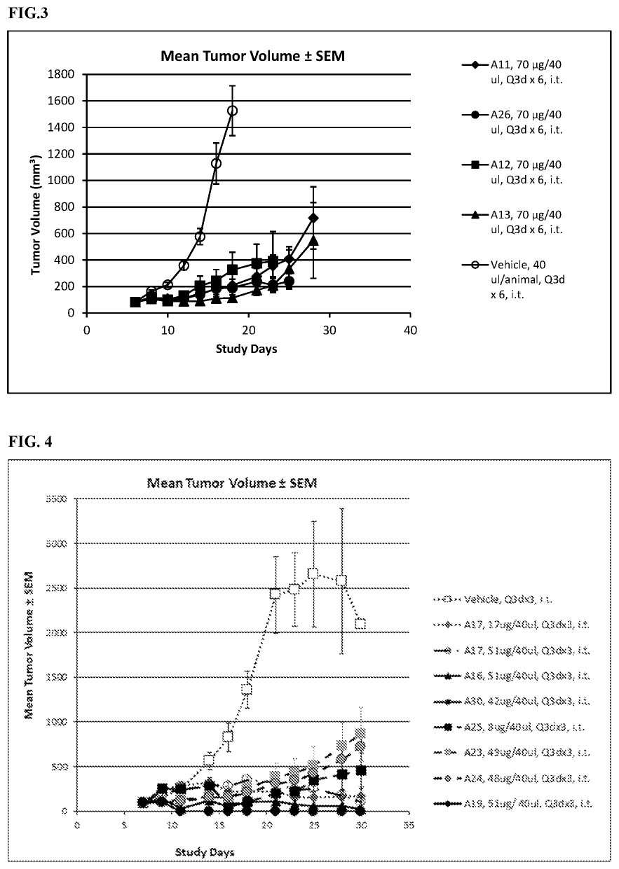

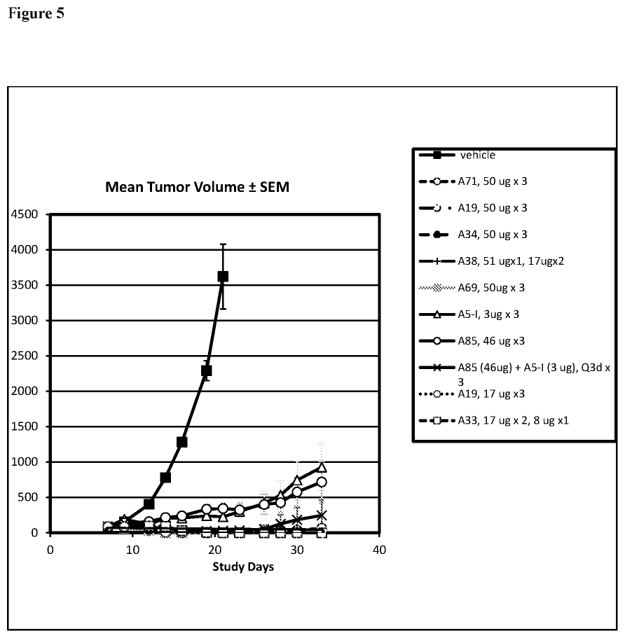

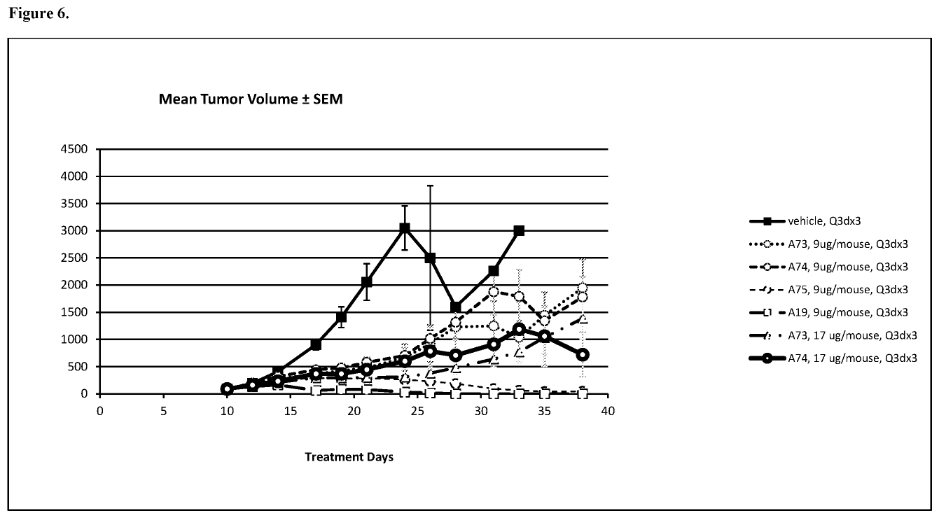

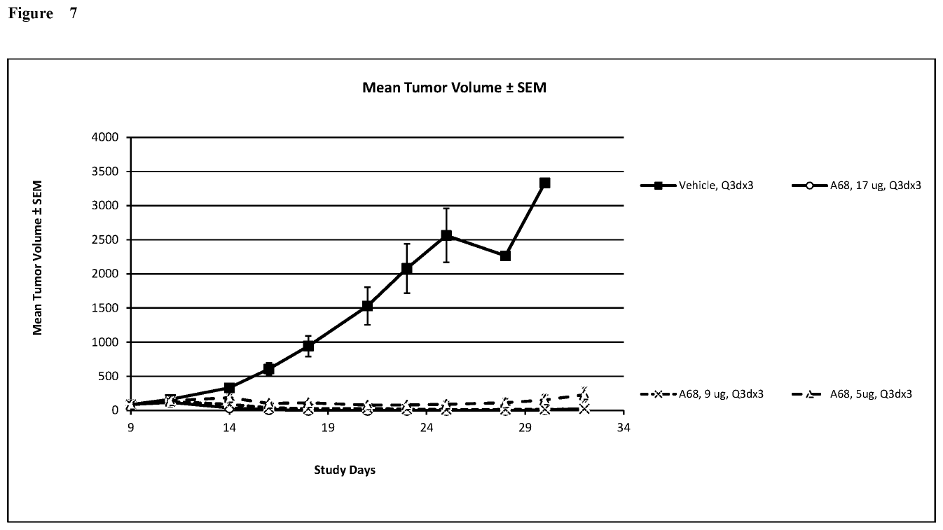

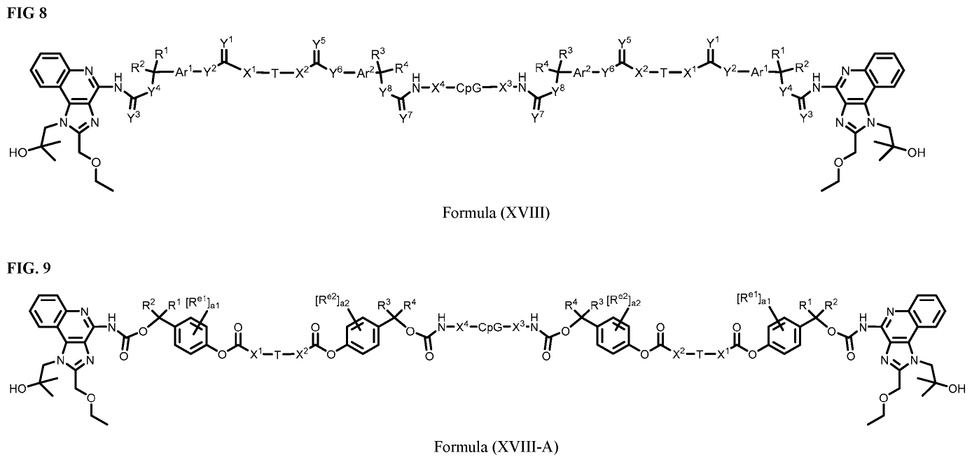

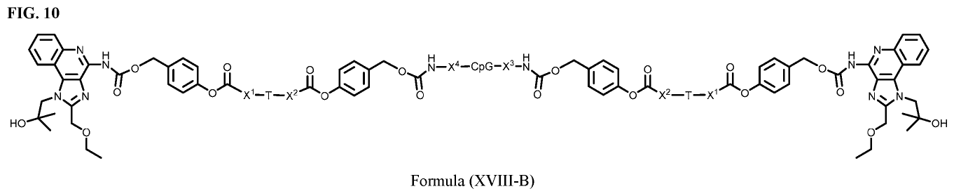

As shown in FIG. 3, compounds A11, A12, A13, and A26 showed significant anti-tumor activity compared to vehicle control group. No significant body weight loss was observed for all treated mice.

### Example 97. Evaluation of Compound A16, A17, A19, A23, A24, A25 and A30 in MC38 Murine Syngeneic Model

To evaluate the in vivo efficacy of conjugates, C57BL/6 mouse was subcutaneously inoculated in the right flank with MC38 tumor cells (1×10⁶) in 40 ul of HBSS for tumor development. Treatment started when tumor size reached around 80-100 mm³, on day 7 after tumor cell implantation. Compounds A16, A17, A19, A23, A24, A25 and A30 were administered by IT injection. The treatment scheme is displayed in Table 4.

TABLE 4
Dosing schedule for Example 97

| Treatment (μg/mouse) | Dosing schedule |
|---|---|
| Compound A16 (51 μg) | Q3d × 3 |
| Compound A17 (17 μg) | Q3d × 3 |
| Compound A17 (51 μg) | Q3d × 3 |
| Compound A19 (51 μg) | Q3d × 3 |
| Compound A23 (49 μg) | Q3d × 3 |
| Compound A24 (48 μg) | Q3d × 3 |
| Compound A25 (8 μg) | Q3d × 3 |
| Compound A30 (42 μg) | Q3d × 3 |

As shown in FIG. 4, compounds A16, A17, A19, A23, A24 and A25 demonstrated significantly improved anti-tumor activity compared to vehicle treated group. No significant body weight loss was observed for all treated mice.

### Example 98. Evaluation of Compound A5-I, A19, A33, A34, A38, A69, A71 and A85 in MC38 Murine Syngeneic Model

To evaluate the in vivo efficacy of conjugates, C57BL/6 mouse was subcutaneously inoculated in the right flank with MC38 tumor cells (1×10⁶) in 40 ul of HBSS for tumor development. Treatment started when tumor size reached around 80-100 mm³, on day 7 after tumor cell implantation. Compounds (listed in Table 5), were administered by IT injection. The treatment scheme is displayed in Table 5.

TABLE 5
Dosing schedule for Example 98

| Treatment (μg/mouse) | Dosing schedule |
|---|---|
| Compound A5-I (3 μg) | Q3d × 3 |
| Compound A19 (50 μg) | Q3d × 3 |
| Compound A19 (17 μg) | Q3d × 3 |
| Compound A33 (17 μg × 2, 8 ug × 1) | Q3d × 3 |
| Compound A34 (50 μg) | Q3d × 3 |
| Compound A38 (51 μg × 1, 17 ug × 2) | Q3d × 3 |
| Compound A69 (50 μg) | Q3d × 3 |
| Compound A71 (50 μg) | Q3d × 3 |
| Compound A85 (46 μg) | Q3d × 3 |
| Compound A5-I (3 μg) + A85 (46 μg) | Q3d × 3 |

As shown in FIG. 5, A5-I, A19, A33, A34, A38, A69, A71, A85 and a mixture of A5-I and A85 demonstrated significantly improved anti-tumor activity compared to vehicle-treated group, STING-TLR9 conjugate A19 showed better anti-tumor activity compared to the mixture of STING agonist A5-I and TLR9 agonists A85, resulting in complete tumor regression for all treated mice. Combination of STING and TLR9 agonists also demonstrated significantly improved anti-tumor activity compared to the treatment of either STING agonist A5-I or TLR9 agonist A85 alone.

### Example 99. Evaluation of Compound A19, A73, A74 and A75 in MC38 Murine Syngeneic Model

To evaluate the in vivo efficacy of conjugates, C57BL/6 mouse was subcutaneously inoculated in the right flank with MC38 tumor cells (1×10⁶) in 40 ul of HBSS for tumor development. Treatment started when tumor size reached around 80-100 mm³, on day 7 after tumor cell implantation. Each compound (listed in Table 6) was administered by IT injection. The treatment scheme is displayed in Table 6.

TABLE 6
Dosing schedule for Example 99

| Treatment (μg/mouse) | Dosing schedule |
|---|---|
| Compound A19 (9 μg) | Q3d × 3 |
| Compound A73 (9 μg) | Q3d × 3 |
| Compound A73 (17 μg) | Q3d × 3 |
| Compound A74 (9 ug) | Q3d × 3 |
| Compound A74 (17 μg) | Q3d × 3 |
| Compound A 75 (9 ug) | Q3d × 3 |

As shown in FIGS. 6, A19, A73, A74 and A75 demonstrated significantly improved anti-tumor activity compared to vehicle-treated group, dose-dependent effects were observed with A73 and A74.

### Example 100. Evaluation of Compound A68 in MC38 Murine Syngeneic Model

To evaluate the in vivo efficacy of conjugates, C57BL/6 mouse was subcutaneously inoculated in the right flank with MC38 tumor cells (1×10⁶) in 40 ul of HBSS for tumor development. Treatment started when tumor size reached around 80-100 mm³, on day 7 after tumor cell implantation. A68 was injected intratumorally, mice were administered every three days for a total of three doses.

As shown in FIG. 7, A68 demonstrated significantly improved anti-tumor activity compared to vehicle-treated group in a dose-dependent manner.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

1. A conjugate of formula (X):
[A²-Z²-T-Z³]_{b2}-A¹-[Z¹-T-Z²-A²]_{b1} (X)
or a tautomer, a mixture of two or more tautomers, or an isotopic variant thereof; or a pharmaceutically acceptable salt, solvate, hydrate, or prodrug thereof;
wherein:
b1 is an integer of 0 or 1;
b2 is an integer of 0 or 1;
wherein b1+b2 is 1 or 2;
each T is independently a triazole functional group;
Z¹, Z², and Z³ are each independently a spacer; and
A¹ and A² are each independently a therapeutic agent or an active moiety of a therapeutic agent; or a compound that decomposes to a therapeutic agent;
wherein one or more atoms or a chemical group in the therapeutic agent or the compound that decomposes to a therapeutic agent is independently replaced with a covalent bond to the spacer.

2.-9. (canceled)

10. The conjugate of claim 1, wherein A¹ and A² are each independently a STING agonist, a cyclic dinucleotide (CDN), a TLR9 agonist, or a TLR7/8 agonist or derivative thereof.

11.-18. (canceled)

19. The conjugate of claim 1, wherein the conjugate has a structure of formula (X-1):
[A²-Z²-T-Z³]_{b2}-CPG-[Z¹-T-Z²-A²]^{b1} (X-1)
wherein:
CpG is a TLR9 agonist oligodeoxynucleotide;
A² is a STING agonist;
Z¹ and Z³ are each independently selected from the group consisting of: 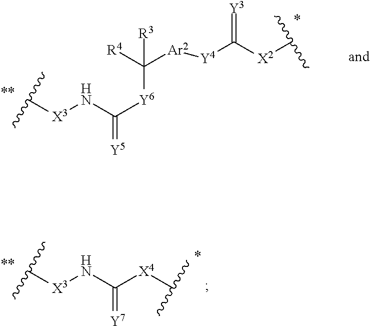
wherein * indicates the attachment point to T and ** indicates the attachment point to a 3′-O or 5′-O of a terminal nucleotide of the CpG;
Z² is selected from the group consisting of: 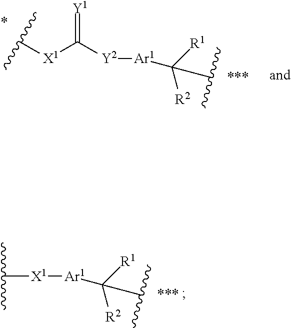
wherein * indicates the attachment point to T and *** indicates the attachment point to the STING agonist;
Ar¹ and Ar² are each independently a substituted or unsubstituted aryl or substituted or unsubstituted heteroaryl;
X¹, X², X³, and X⁴ are each independently a spacer;
R¹, R², R³ and R⁴ are each independently a hydrogen, substituted or unsubstituted alkyl, substituted or unsubstituted alkenyl, substituted or unsubstituted alkynyl, or substituted or unsubstituted aryl; or
R¹ and R², or R³ and R⁴ together with the atom to which they are attached, can join together to form a substituted or unsubstituted 3-8 membered ring that can optionally contain one or two heteroatoms; and
Y¹, Y², Y³, Y⁴, Y⁵, Y⁶, and Y⁷ are each independently O or S.

20.-33. (canceled)

34. The conjugate of claim 10, wherein the STING agonist is ADU-S100, MK-1454, BMS-986301, GSK3745417, E7766, SB11285,

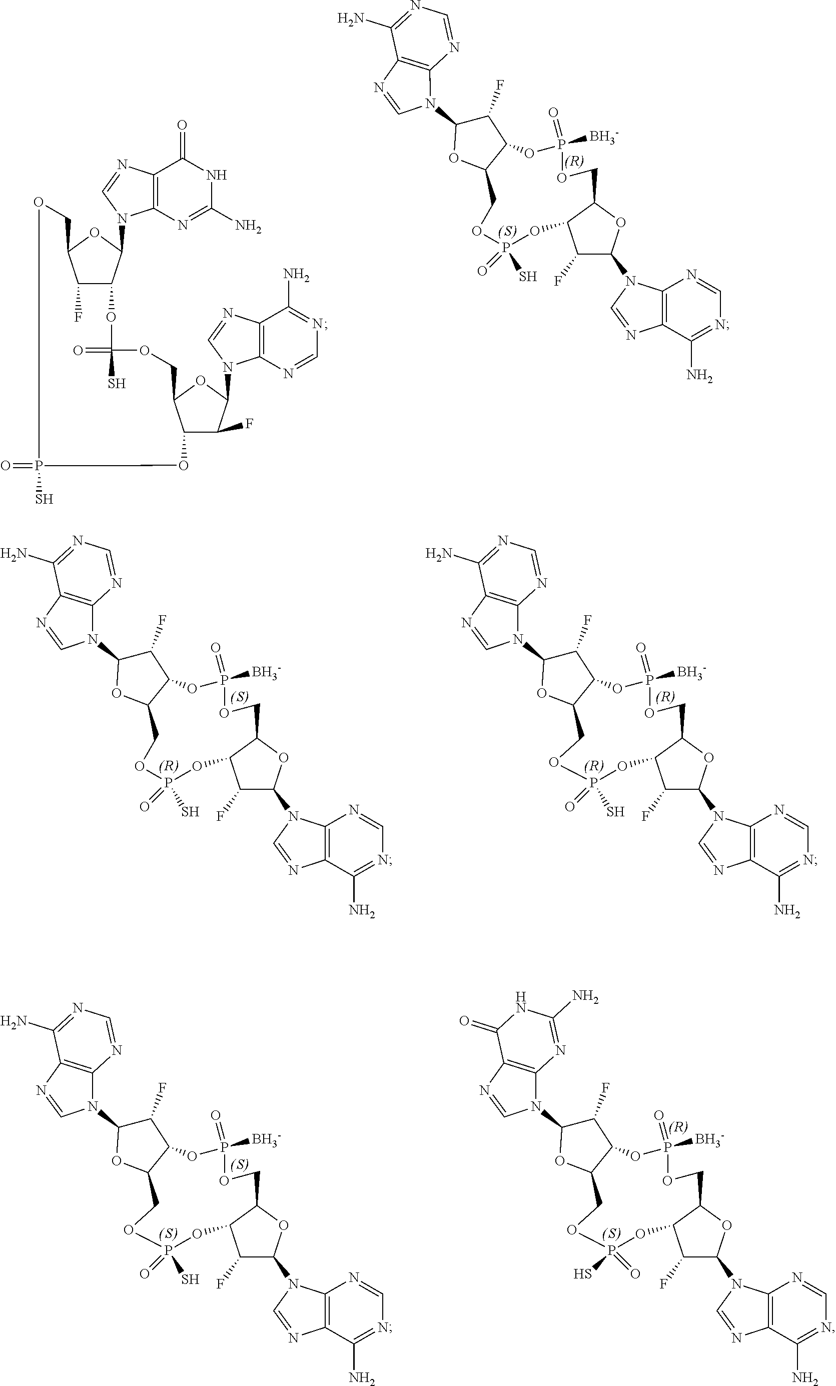

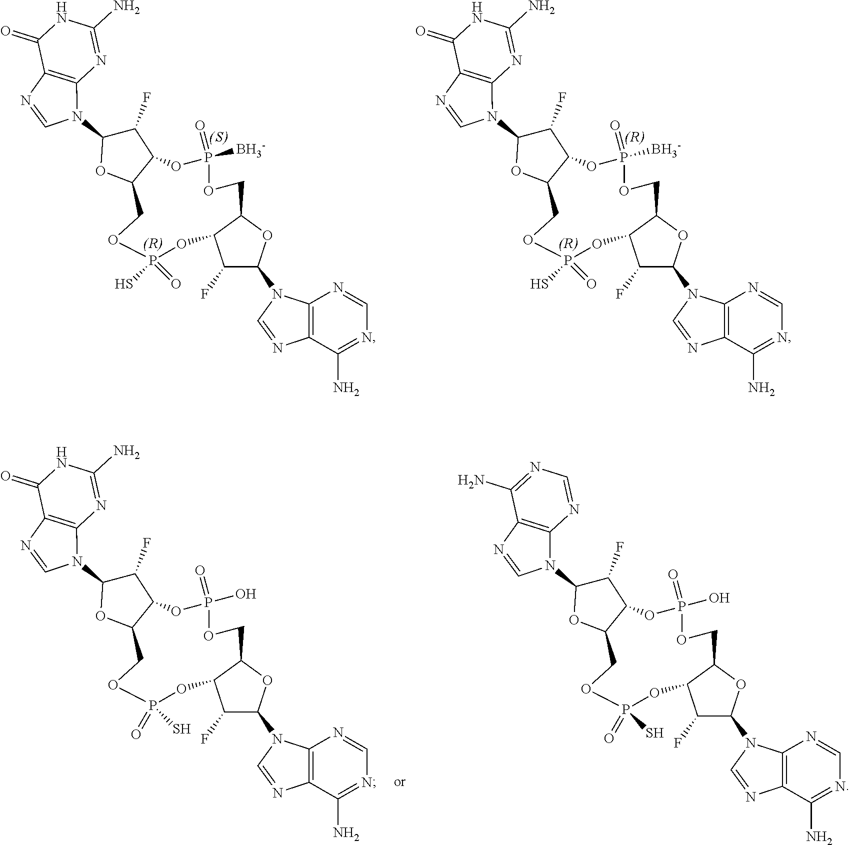

35. The conjugate of claim 1, wherein the conjugate has a structure of formula (XI): 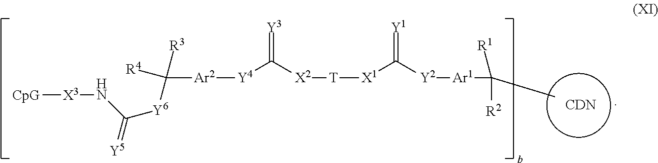
wherein:
b is an integer of 1 or 2; wherein when b=2, both groups are directly bound to CDN;
CDN is a cyclic dinucleotide that is a STING agonist; and
the linker is covalently bound to a thiol group of the cyclic dinucleotide STING agonist.

36. (canceled)

37. The conjugate of claim 35, wherein the conjugate has a structure of formula (XI-B): 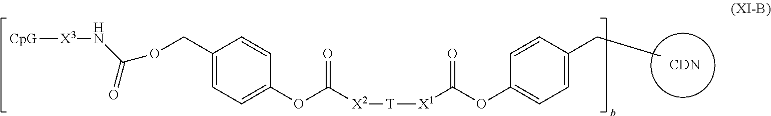
wherein:
X¹ is 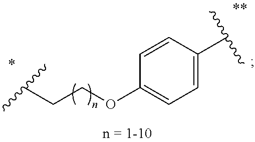
wherein * indicates the attachment point connecting to T, and ** indicates the attachment point connecting to the carbonyl group;
X² is 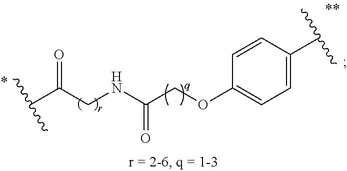
wherein * indicates the attachment point connecting to T, and ** indicates the attachment point connecting to the carbonyl group;
X³ is 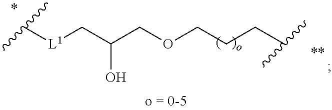
and
wherein L¹ is independently —OP(O)(OH)—, or —OP(S)(OH)—; * indicates the attachment point connecting to a 3′-O terminal nucleotide of the CpG; and ** indicates the attachment point connecting to the amino group.

38. The conjugate of claim 1, wherein the conjugate has a structure of formula (XXVI): 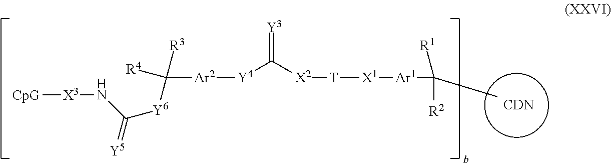
wherein:
CDN is a cyclic dinucleotide that is a STING agonist; and
the linker is covalently bound to a thiol group of the cyclic dinucleotide STING agonist.

39. (canceled)

40. The conjugate of claim 38, wherein the conjugate has a structure of formula (XXVI-B): 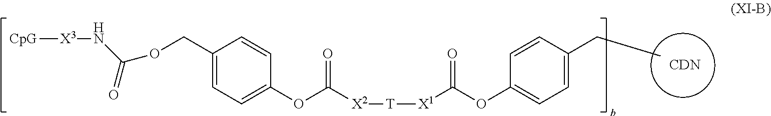
wherein:
X¹ is: 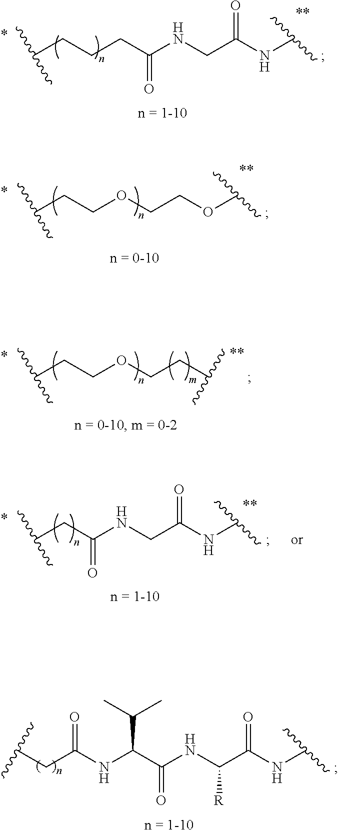
wherein R is methyl or 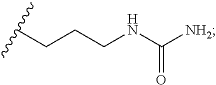 [SMILES: *CCCNC(N)=O]
* indicates the attachment point connecting to selected T, and ** indicates the attachment point connecting to the phenyl group;
X² is 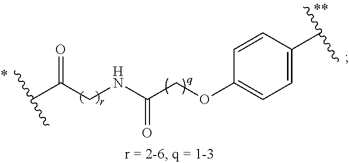
wherein * indicates the attachment point connecting to T, and ** indicates the attachment point connecting to the carbonyl group; and
X³ is 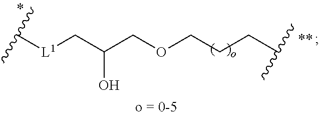
wherein L¹ is independently —OP(O)(OH)—, or —OP(S)(OH)—, * indicates the attachment point connecting to a 3′-O of a terminal nucleotide of the CpG; ** indicates the attachment point connecting to the amino group.

41. (canceled)

42. The conjugate of claim 1, wherein the conjugate has a structure of formula (XII): 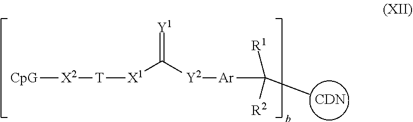
wherein:
b is an integer of 1 or 2; wherein when b=2, both groups are directly bound to CDN;
CDN is a cyclic dinucleotide that is a STING agonist; and
the linker is covalently bound to a thiol group of the cyclic dinucleotide STING agonist.

43. (canceled)

44. The conjugate of claim 42, wherein the conjugate has a structure according to formula (XII-B): 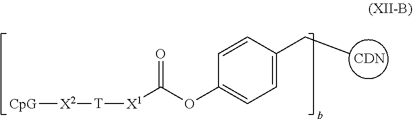
wherein:
X¹ is 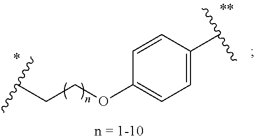
wherein * indicates the attachment point connecting to T, and ** indicates the attachment point connecting to the carbonyl group;
X² is-X³—NH—CO—X⁴—, wherein X³ is 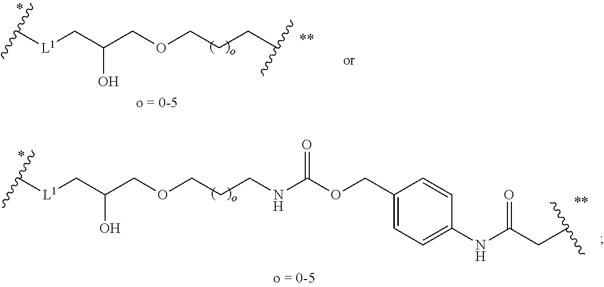
wherein L¹ is independently —OP(O)(OH)—, or —OP(S)(OH)—; * indicates the attachment point connecting to a 3′-O of a terminal nucleotide of the CpG; and ** indicates the attachment point connecting to the amino group; or X³ is: **—C₃-C₁₂ alkylene-L¹-*; wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; and
X⁴ is: 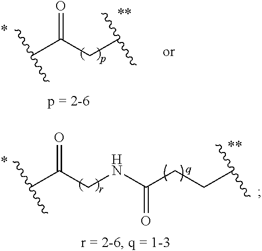
wherein * indicates the attachment point connecting to T, and ** indicates the attachment point connecting to the carbonyl group.

45. The conjugate of claim 1, wherein the conjugate has a structure of formula (XXVII): 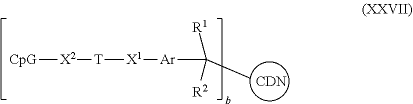
wherein:
CDN is a cyclic dinucleotide that is a STING agonist; and
the linker is covalently bound to a thiol group of the cyclic dinucleotide STING agonist.

46. (canceled)

47. The conjugate of claim 45, wherein the conjugate has a structure of formula (XXVII-B): 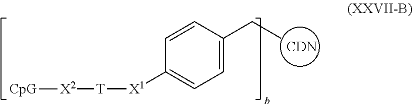
wherein:
X¹ is: 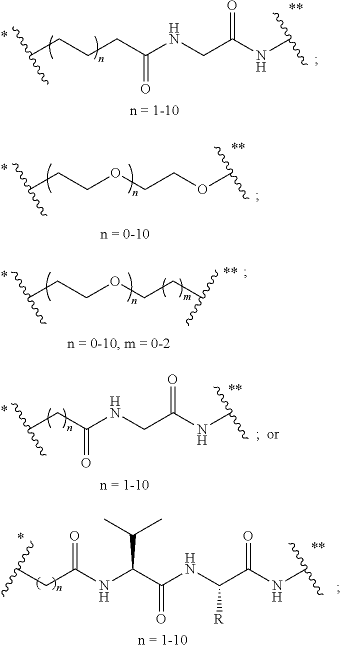
wherein R is methyl or 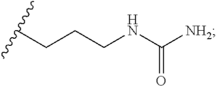 [SMILES: *CCCNC(N)=O]
* indicates the attachment point connecting to T, ** indicates the attachment point connecting to the phenyl group;
X² is-X³—NH—CO—X⁴—, wherein X³ is: 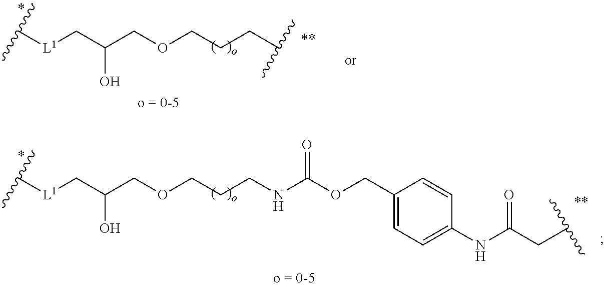
wherein L¹ is independently —OP(O)(OH)—, or —OP(S)(OH)—; * indicates the attachment point connecting to a 3′-O of a terminal nucleotide of the CpG; and ** indicates the attachment point connecting to the amino group; or X³ is: **—C₃-C₁₂ alkylene-L¹-*; or 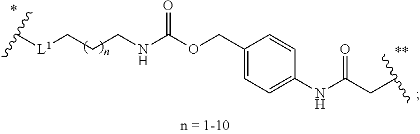
wherein L¹ is independently —OP(O)(OH)—, or —OP(S)(OH)—; * indicates the attachment point connecting to a 5′-O of a terminal nucleotide of the CpG; and ** indicates the attachment point connecting to the amino group; and
X⁴ is: 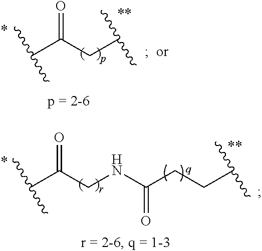
wherein * indicates the attachment point connecting to T, and ** indicates the attachment point connecting to the carbonyl group.

48.-67. (canceled)

68. The conjugate of claim 10, wherein the cyclic dinucleotide is selected from the group consisting of: 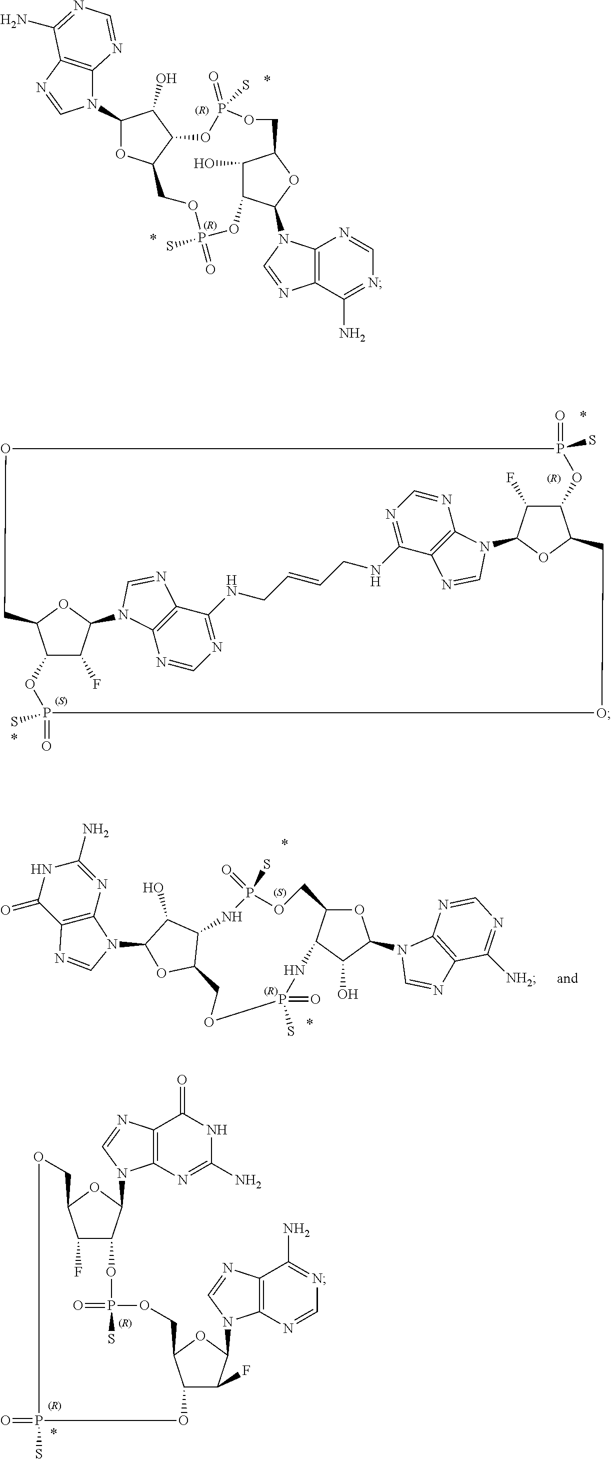
wherein the * indicates the S atom of the cyclic dinucleotide that is connecting with the linker;
wherein:
when b is 1, one S* of the cyclic dinucleotide is connected to a linker and the other S* is connected to a hydrogen; and
when b is 2, both S* of the cyclic dinucleotide are connected to a linker.

69.-70. (canceled)

71. The conjugate of claim 10, wherein the cyclic dinucleotide is selected from the group consisting of: 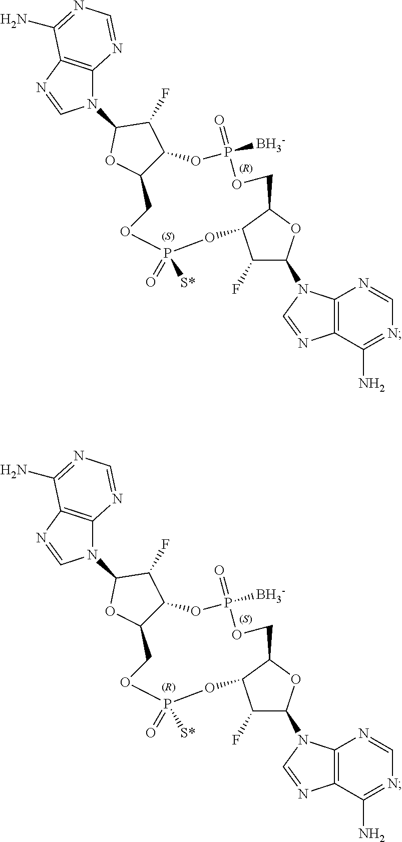 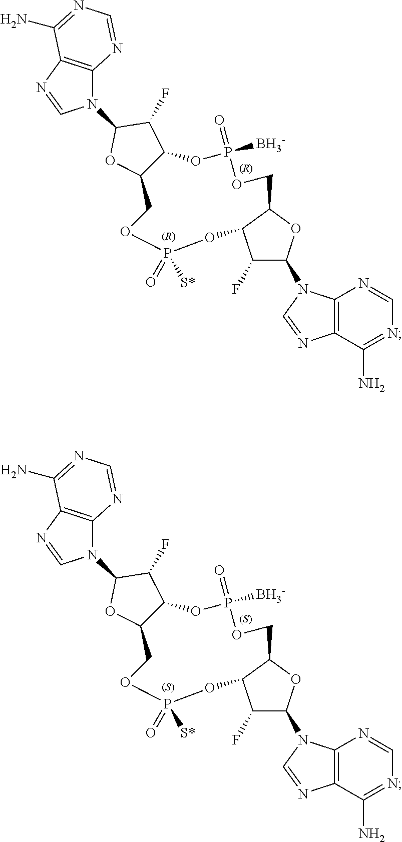 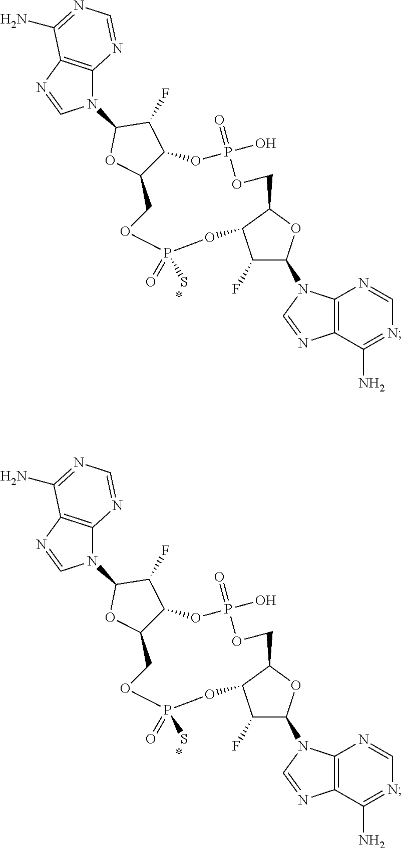 [SMILES: Nc1ncnc2c1ncn2[C@@H]1O[C@@H]2CO[P@@](=O)(S)O[C@H]3[C@@H](F)[C@H](n4cnc5c(N)ncnc54)O[C@@H]3COP(=O)(O)O[C@H]2[C@H]1F ; Nc1ncnc2c1ncn2[C@@H]1O[C@@H]2CO[P@](=O)(S)O[C@H]3[C@@H](F)[C@H](n4cnc5c(N)ncnc54)O[C@@H]3COP(=O)(O)O[C@H]2[C@H]1F] 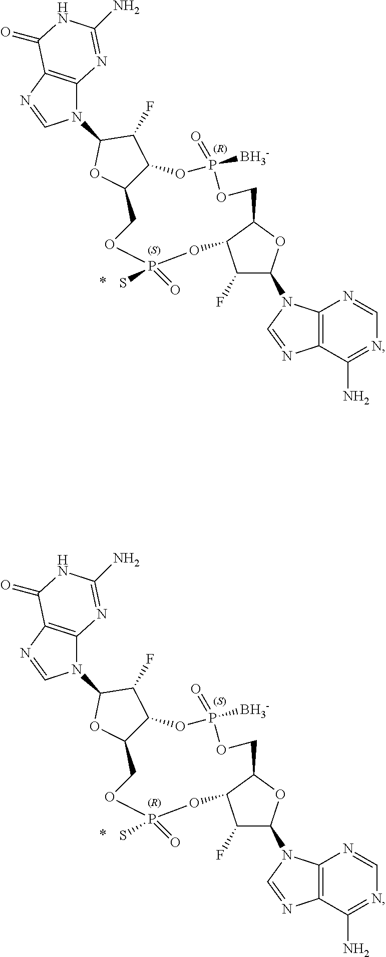 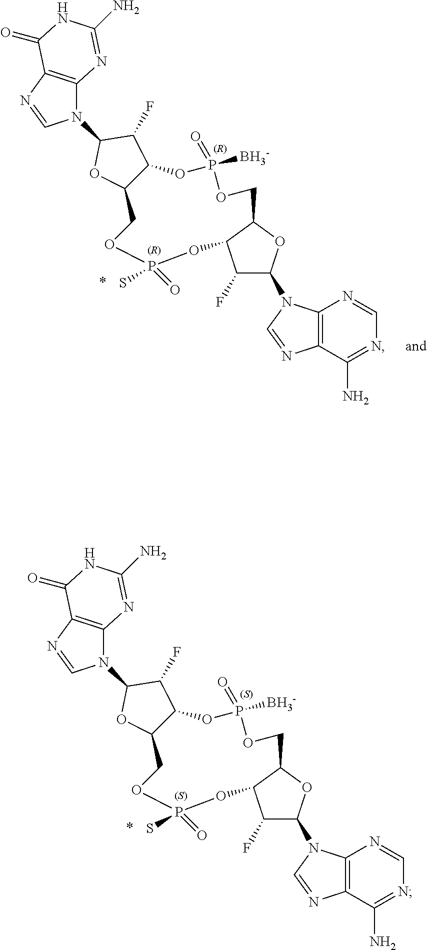
wherein the * indicates the S atom of the cyclic dinucleotide that is connecting with the linker.

72. The conjugate of claim 71, wherein the conjugate has the following structure: 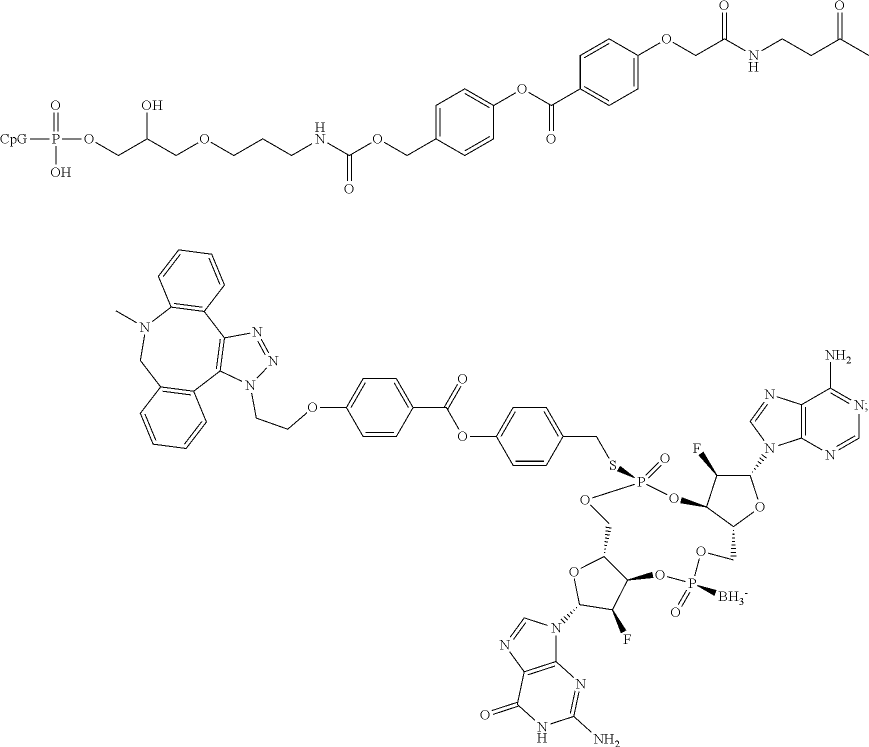 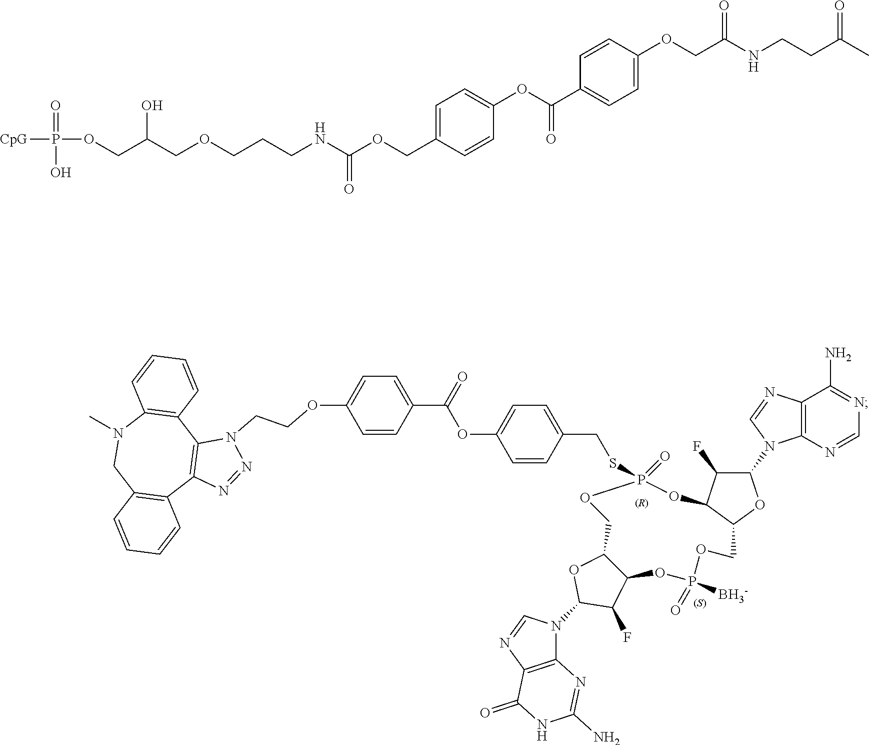 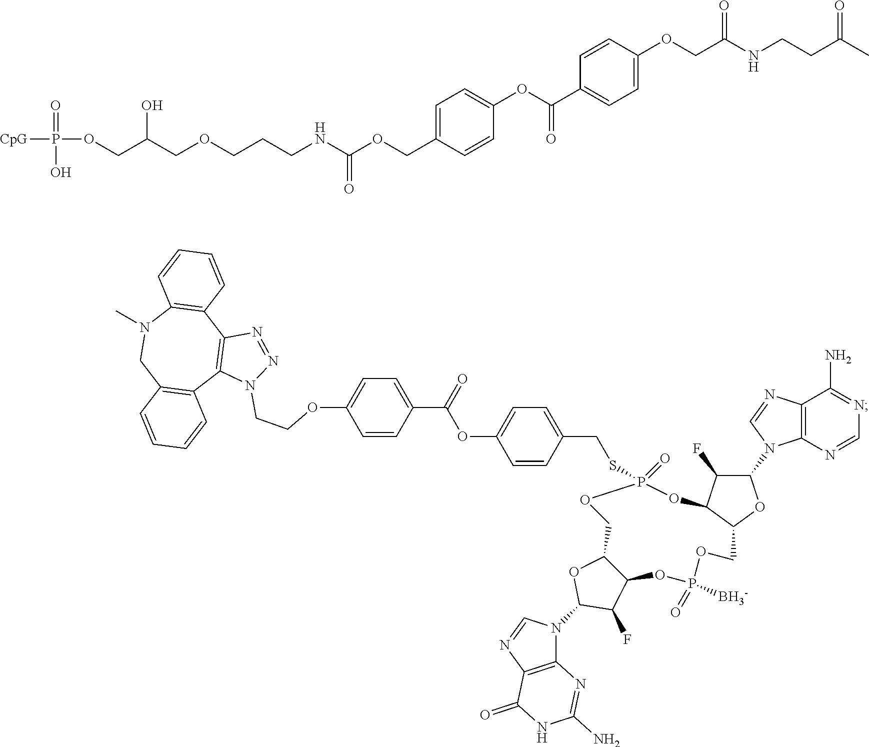 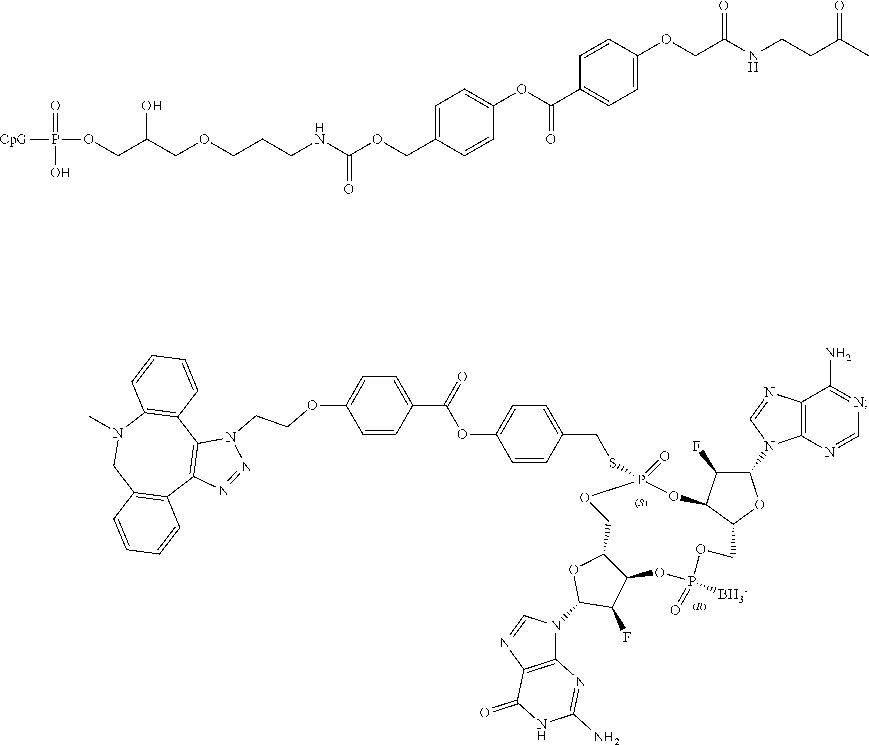 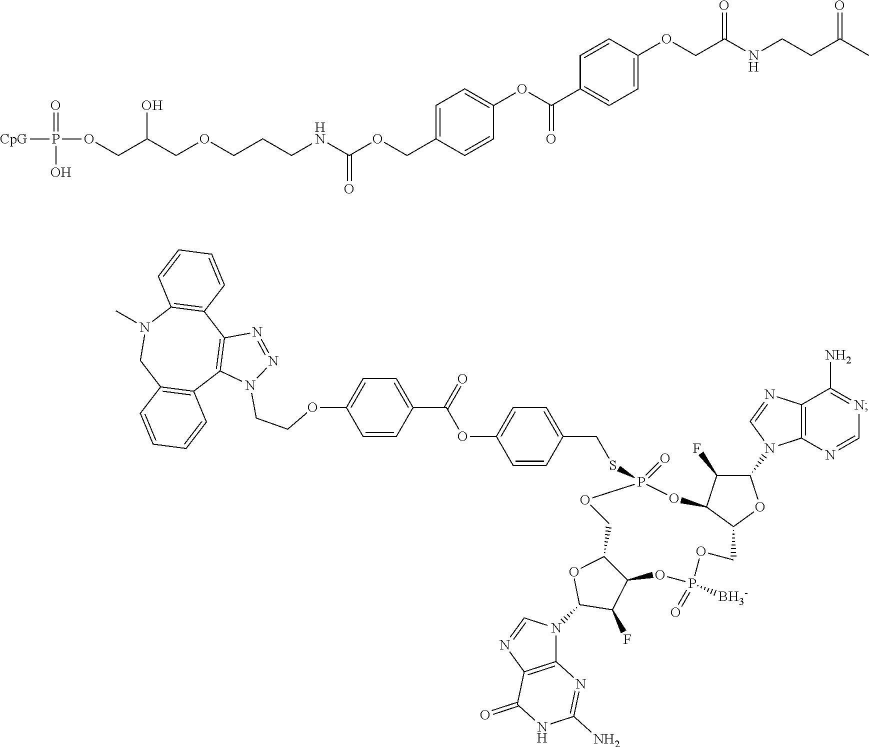 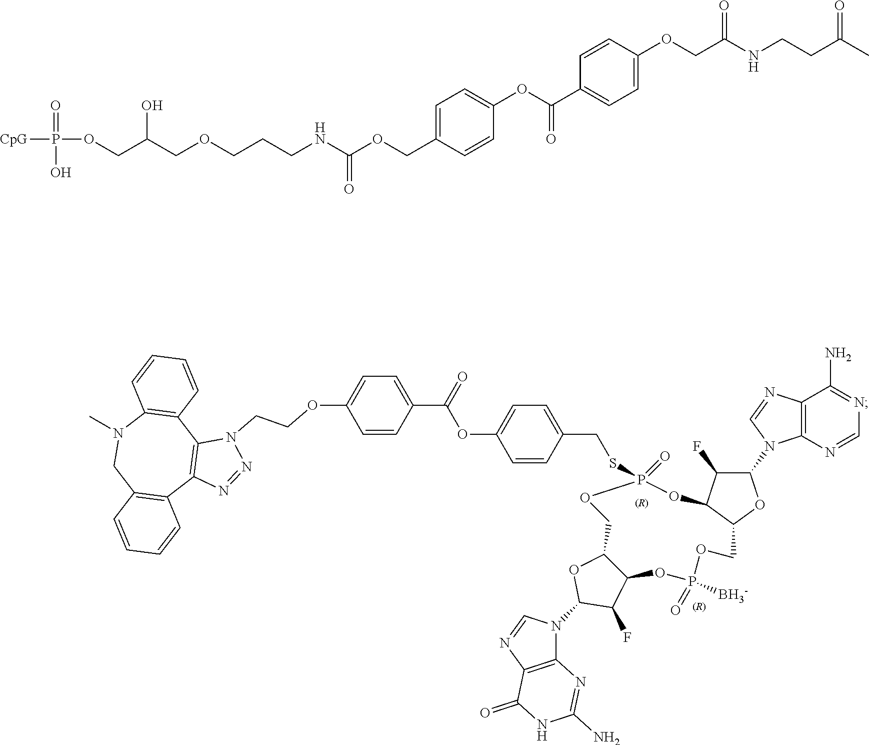 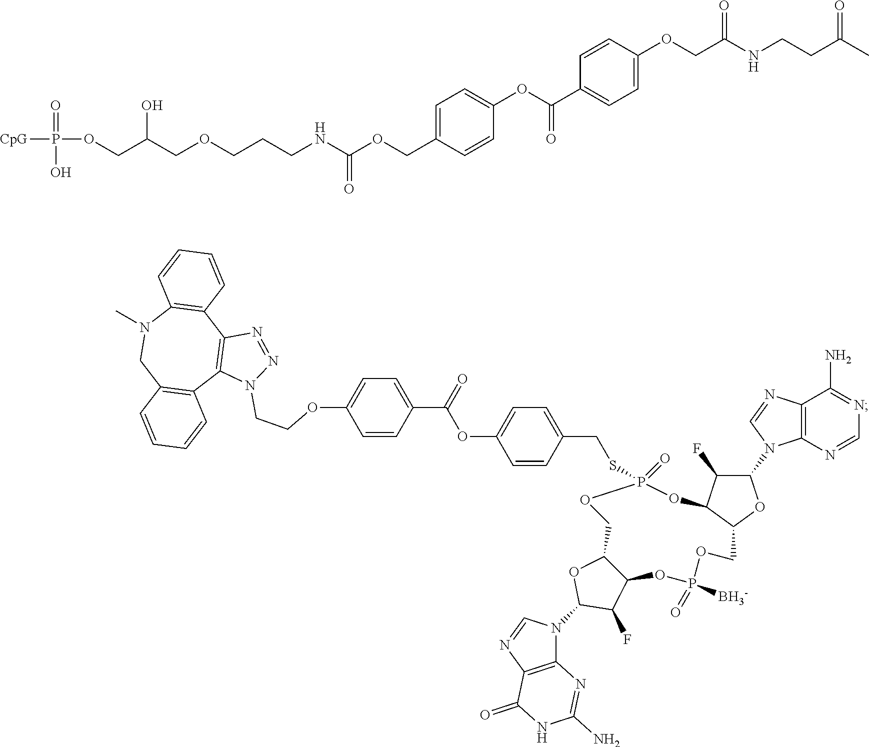 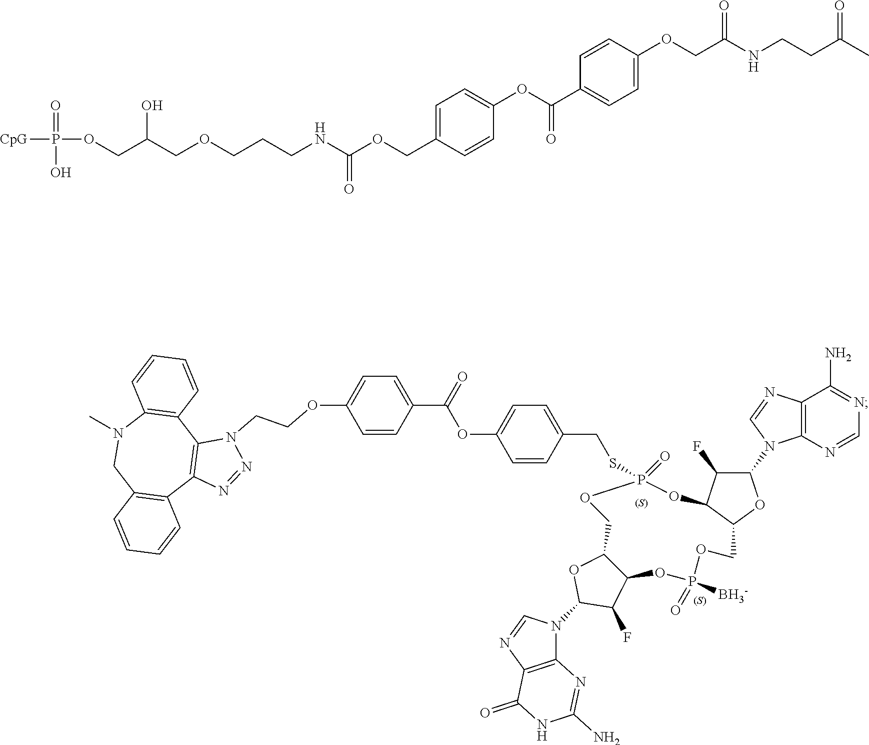 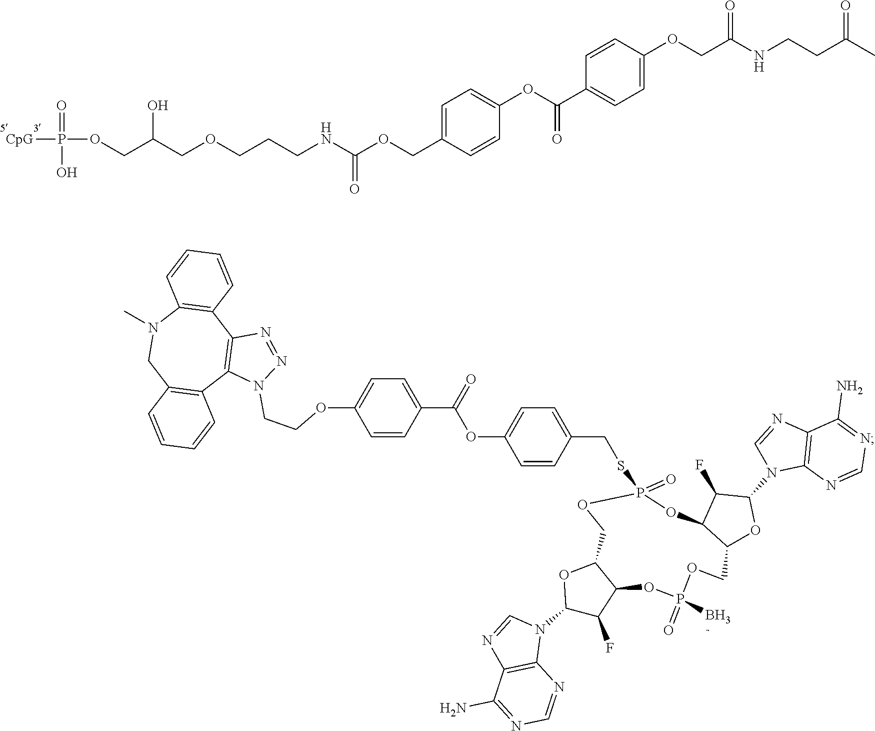 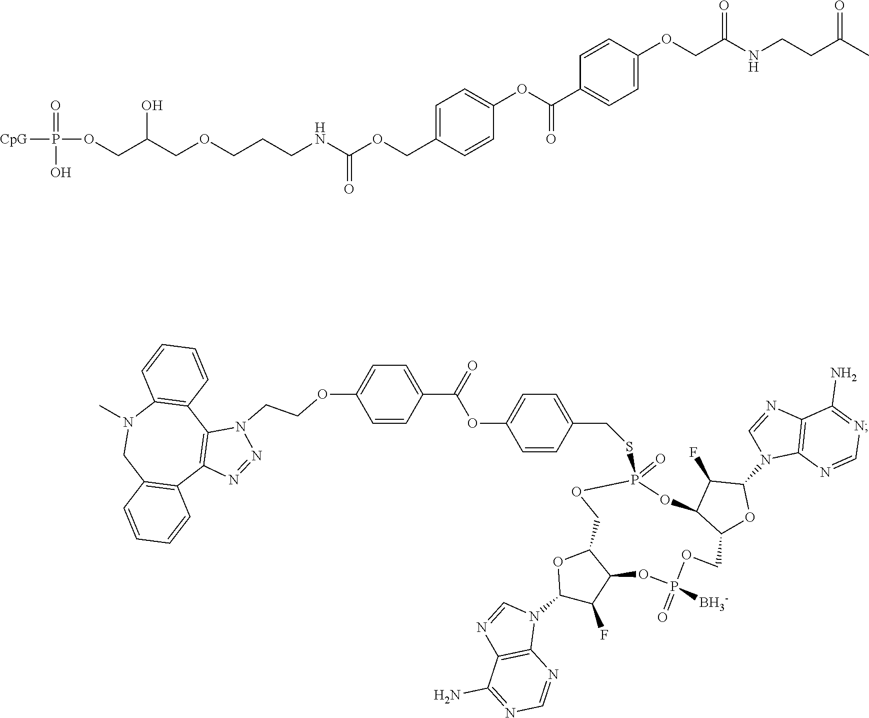 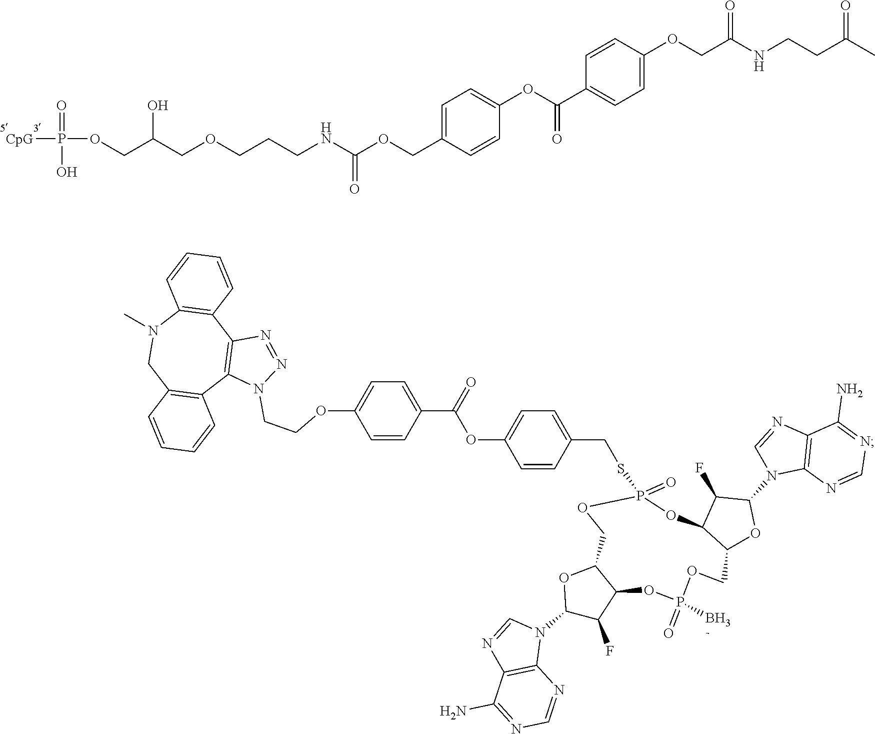 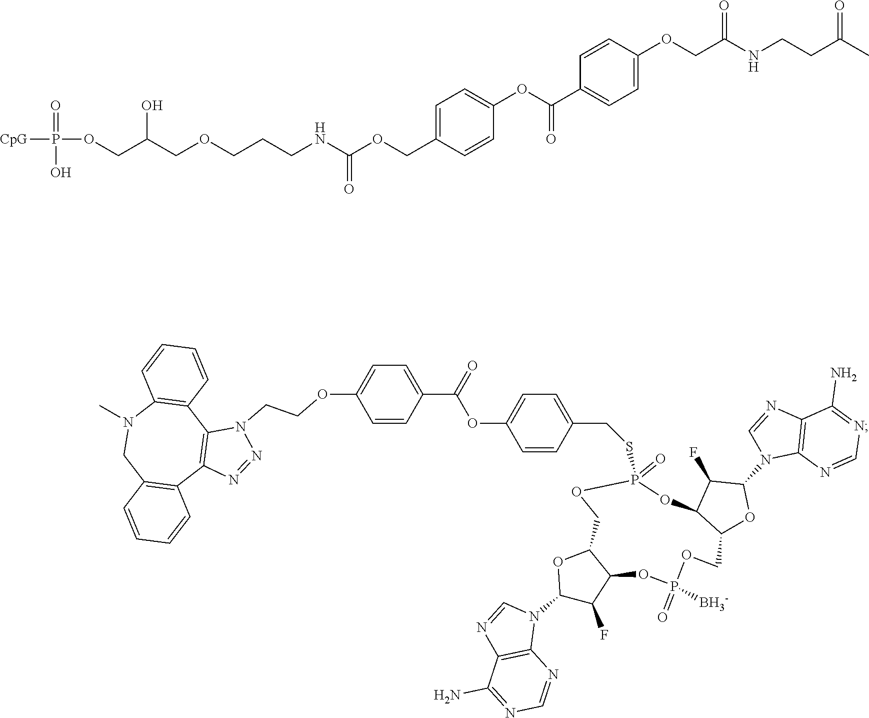 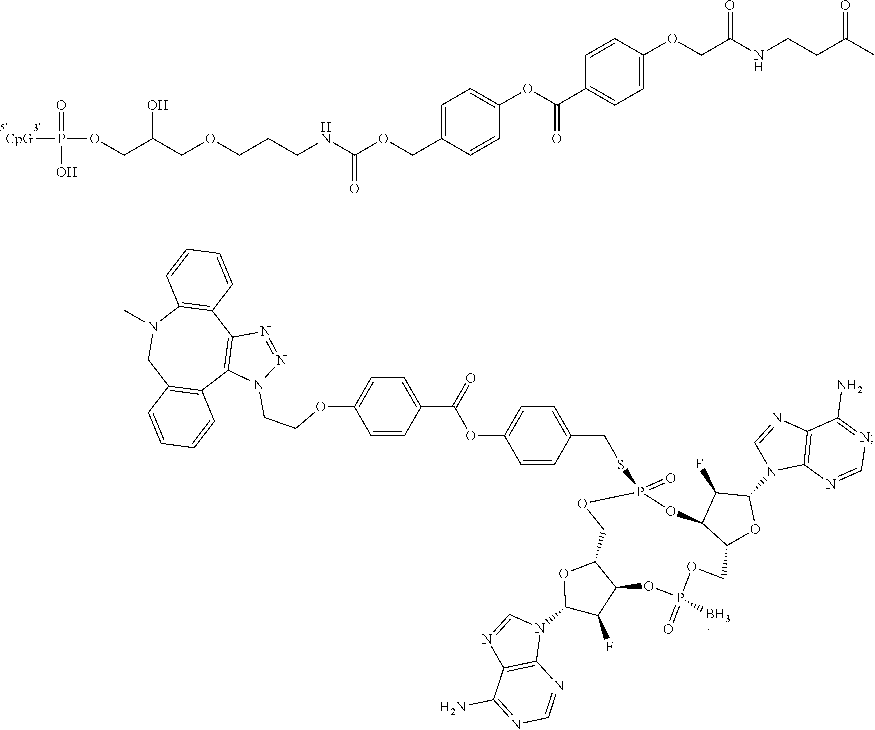 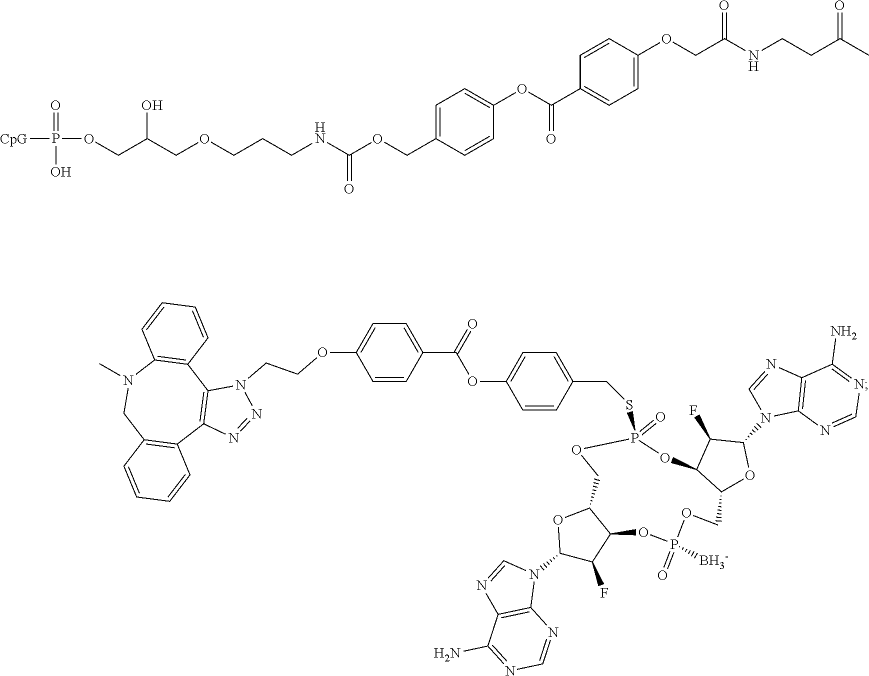 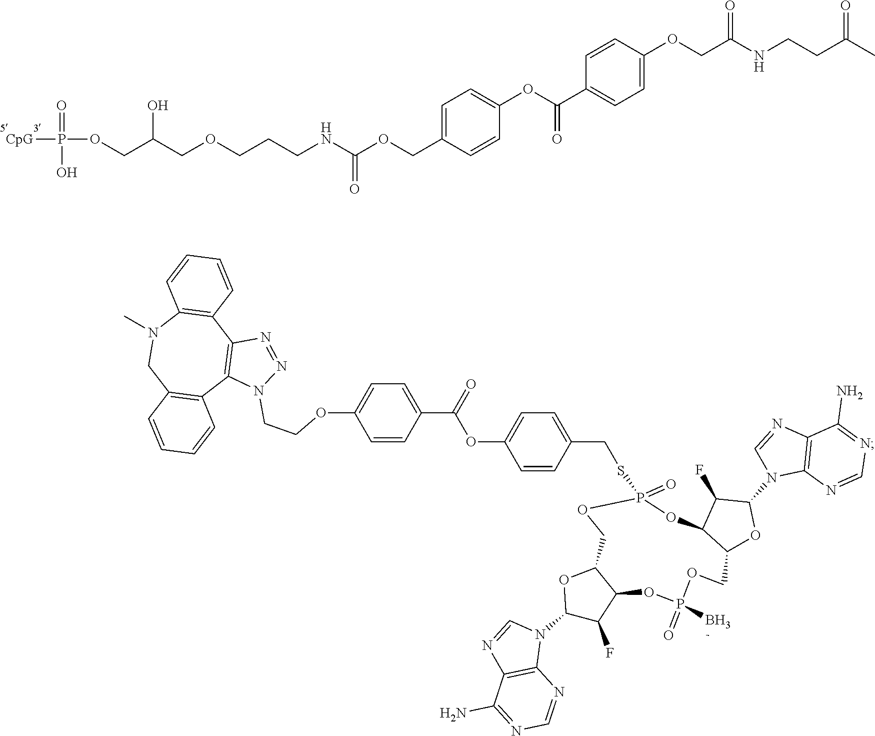 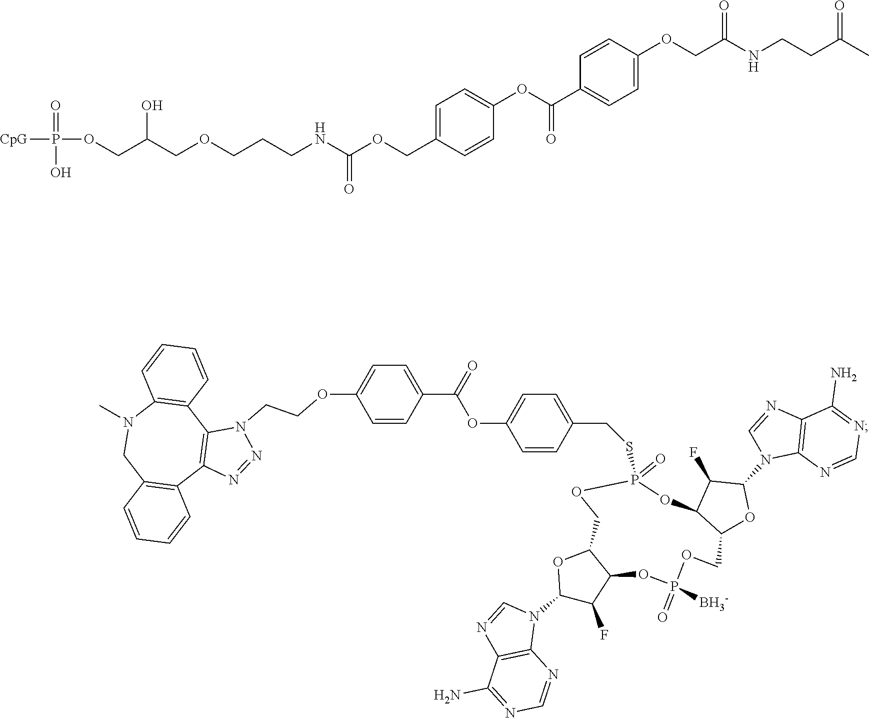 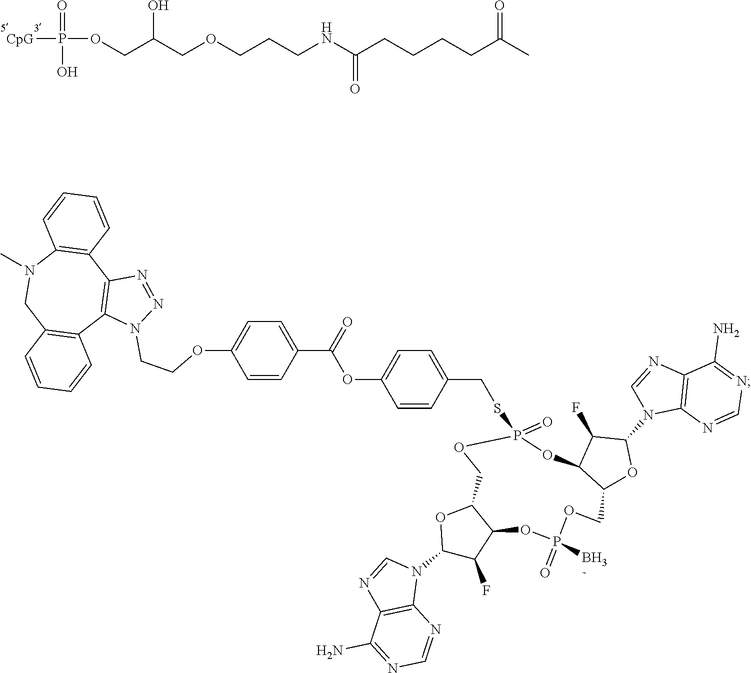 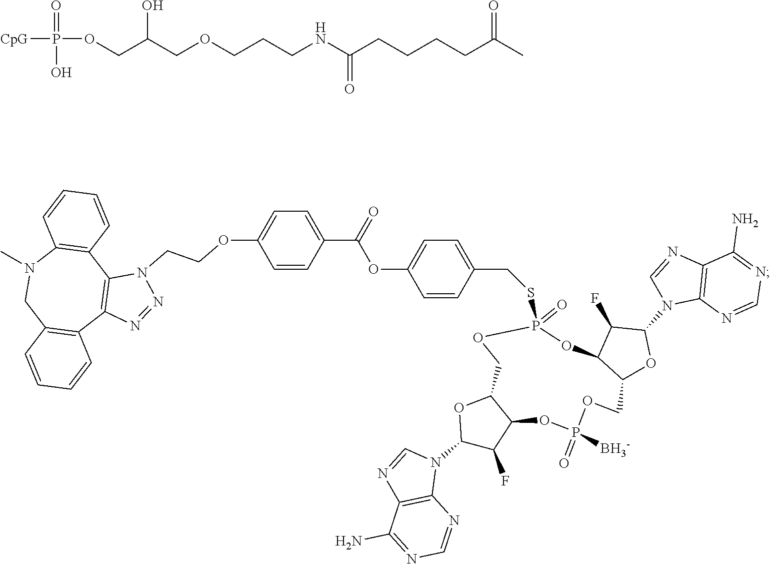 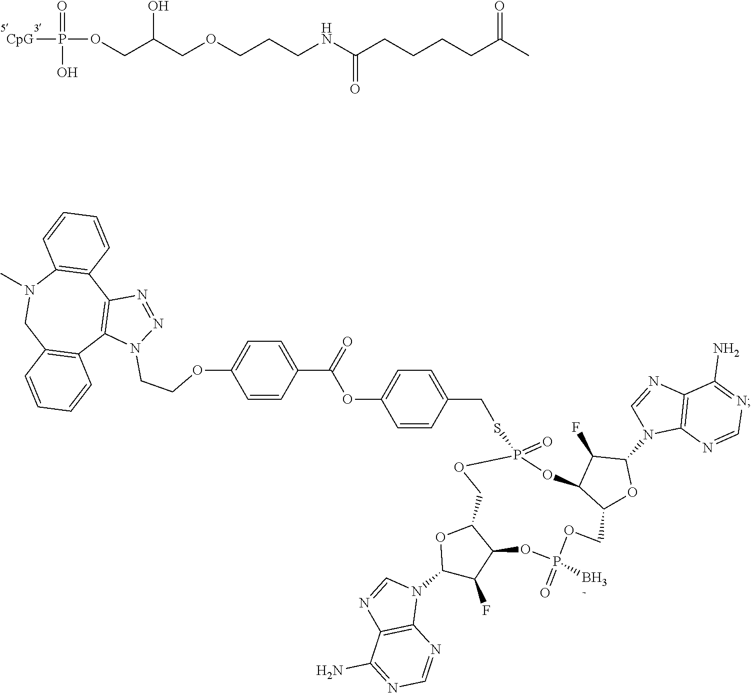 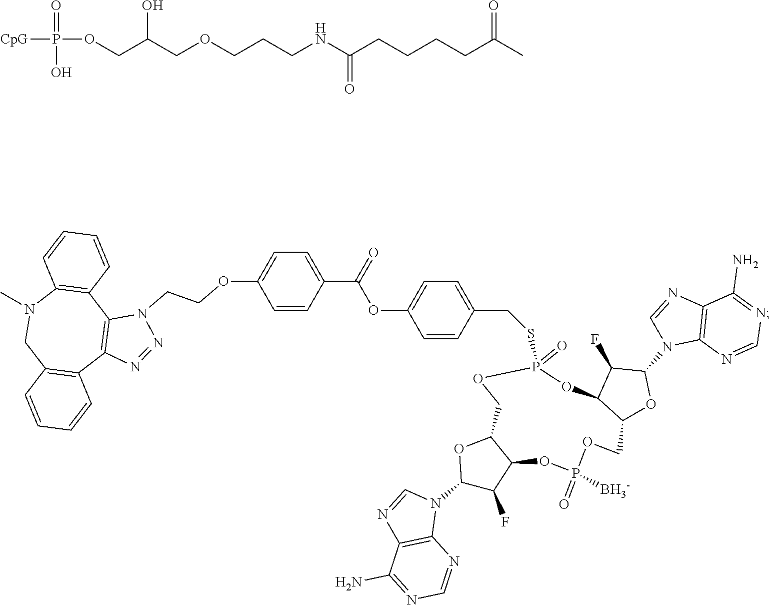 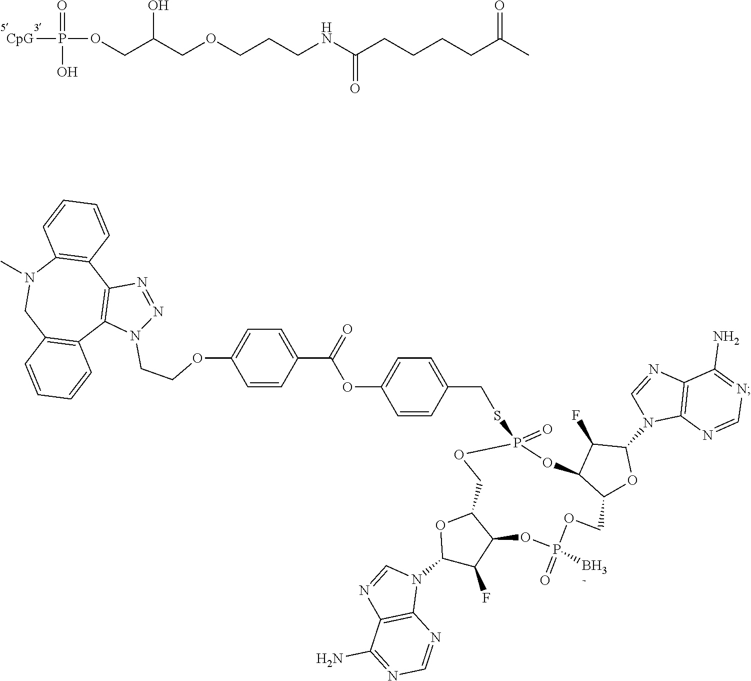 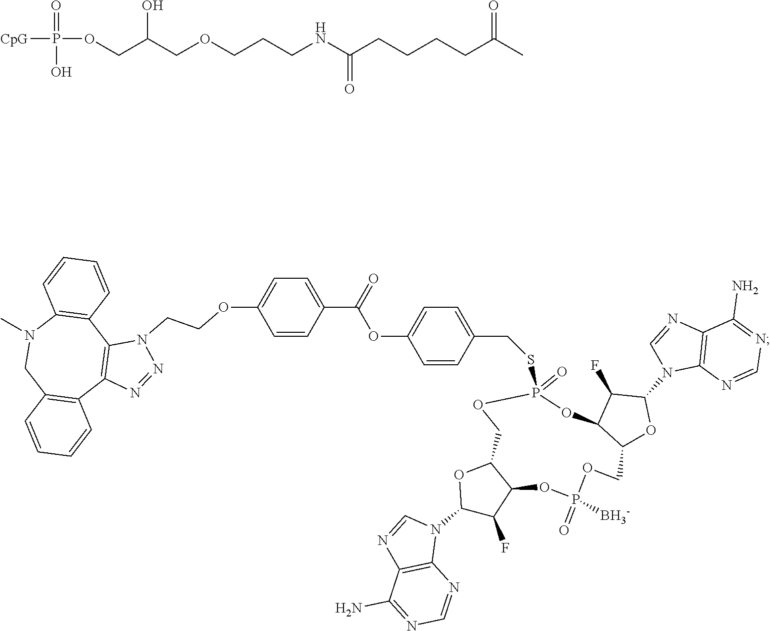 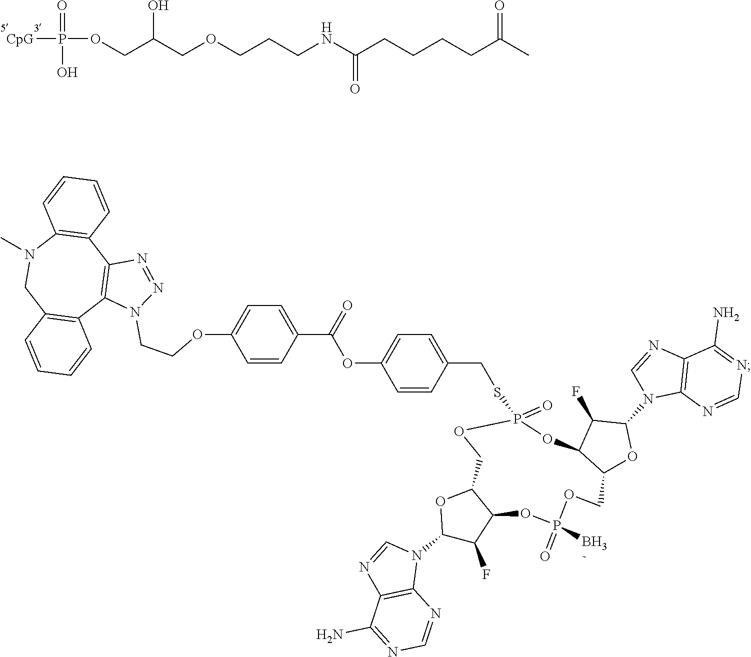 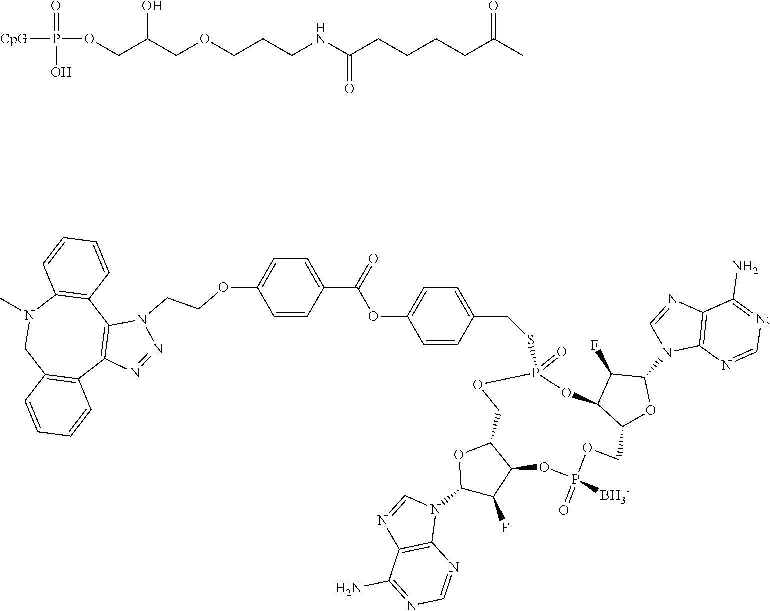 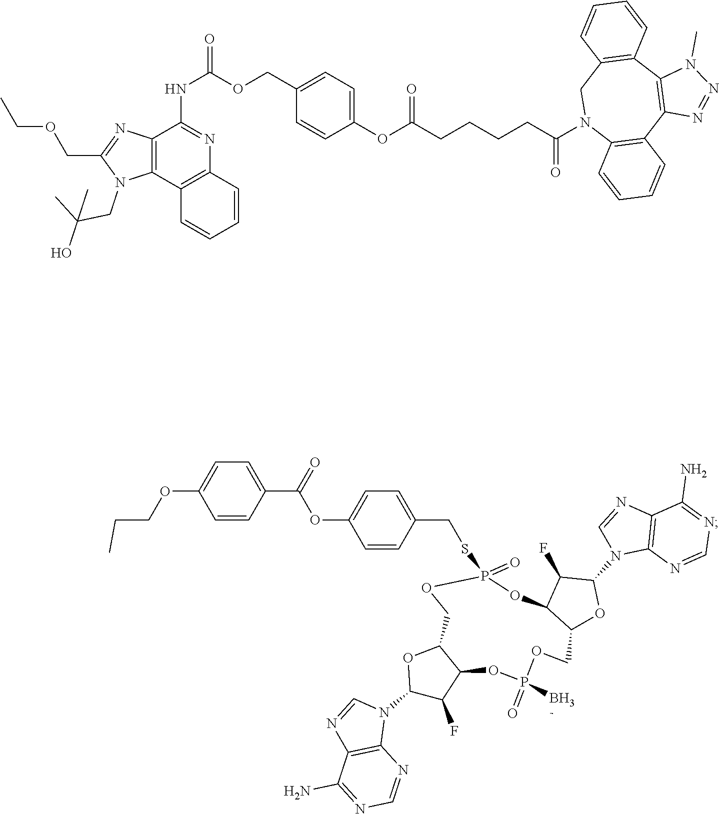 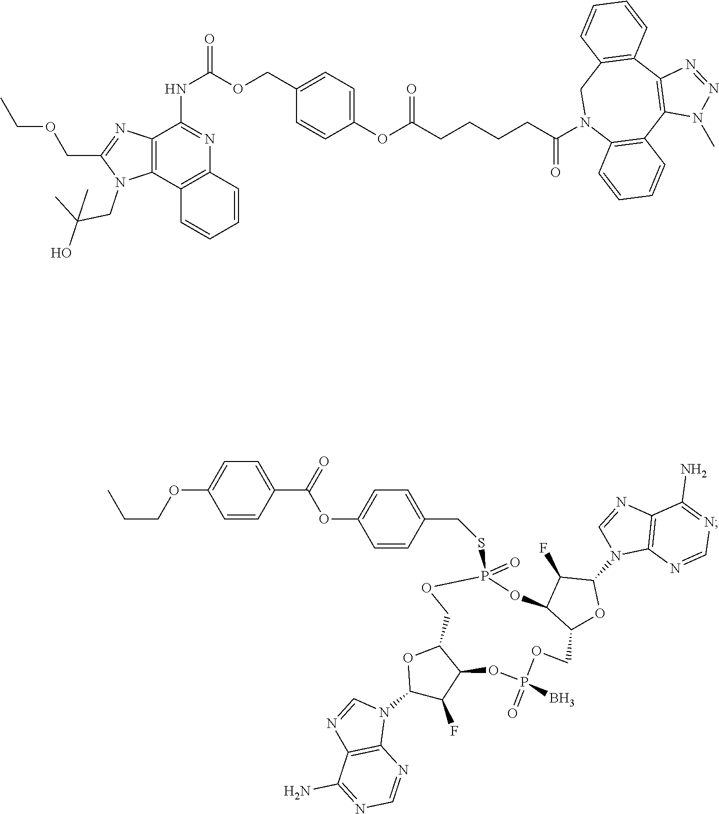 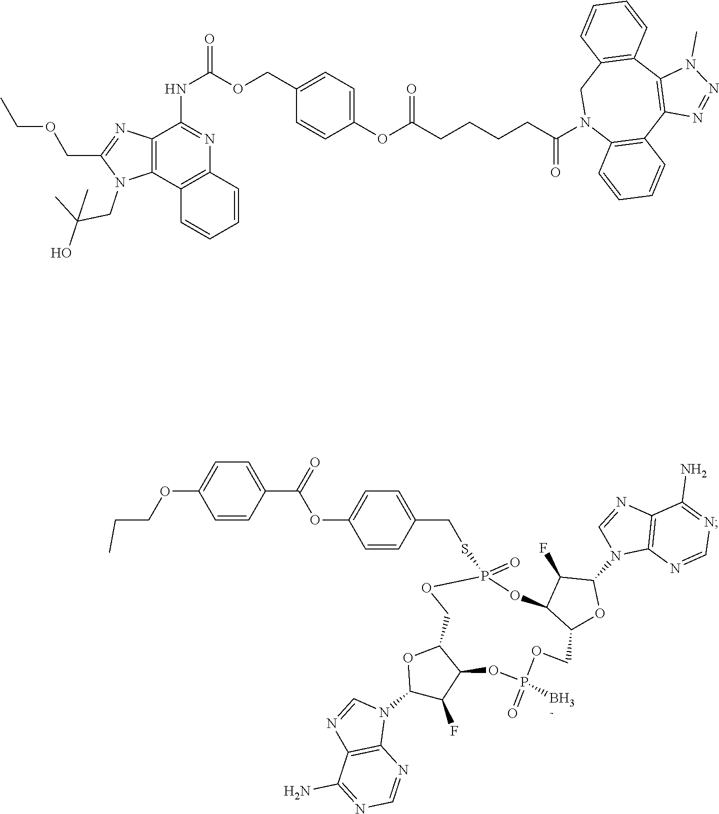 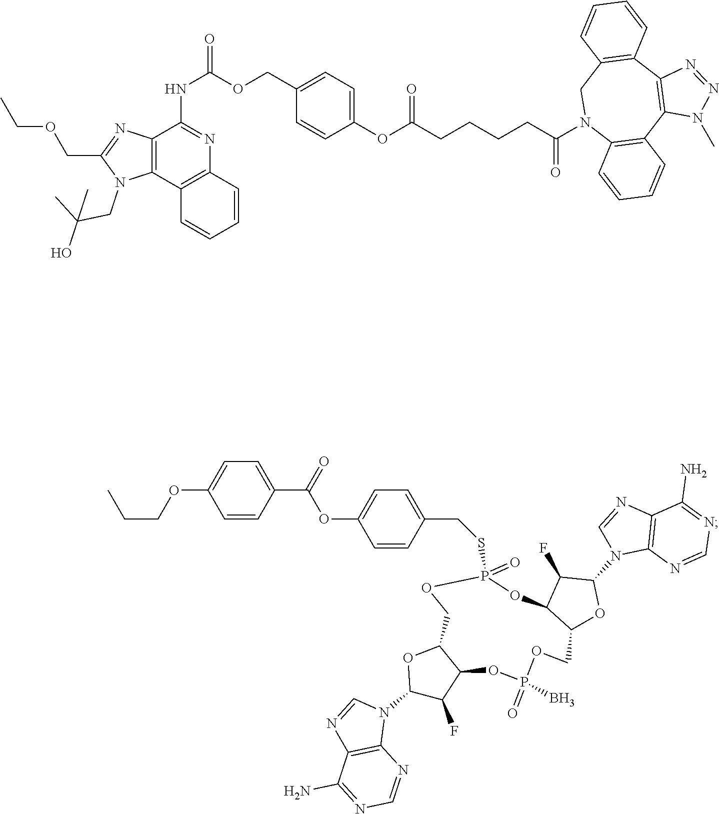 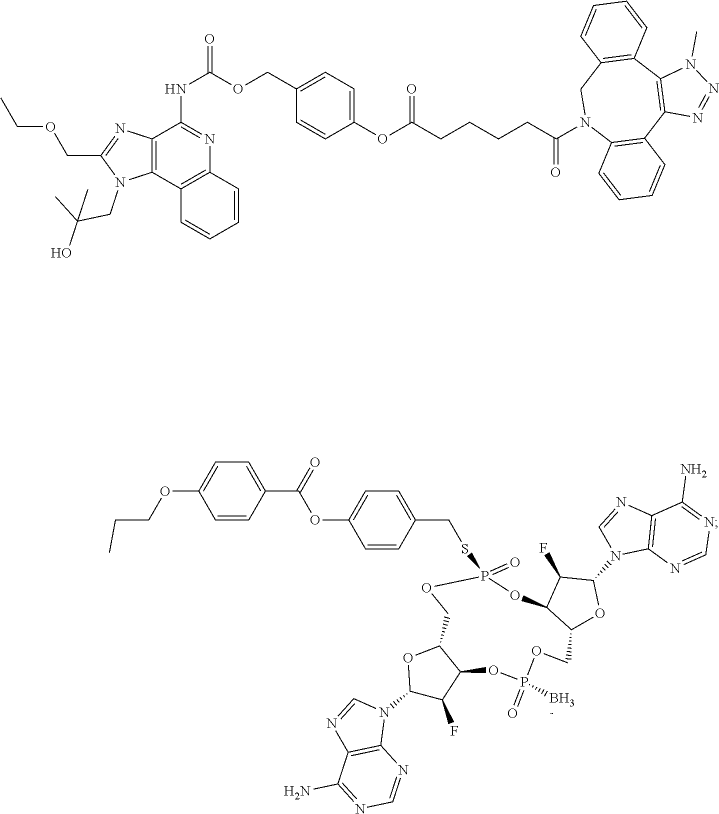 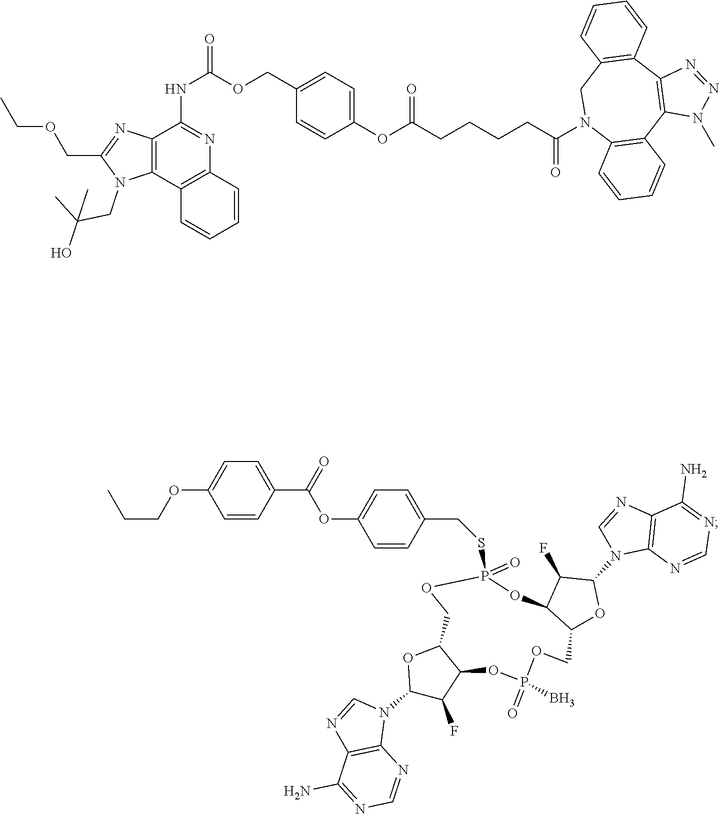 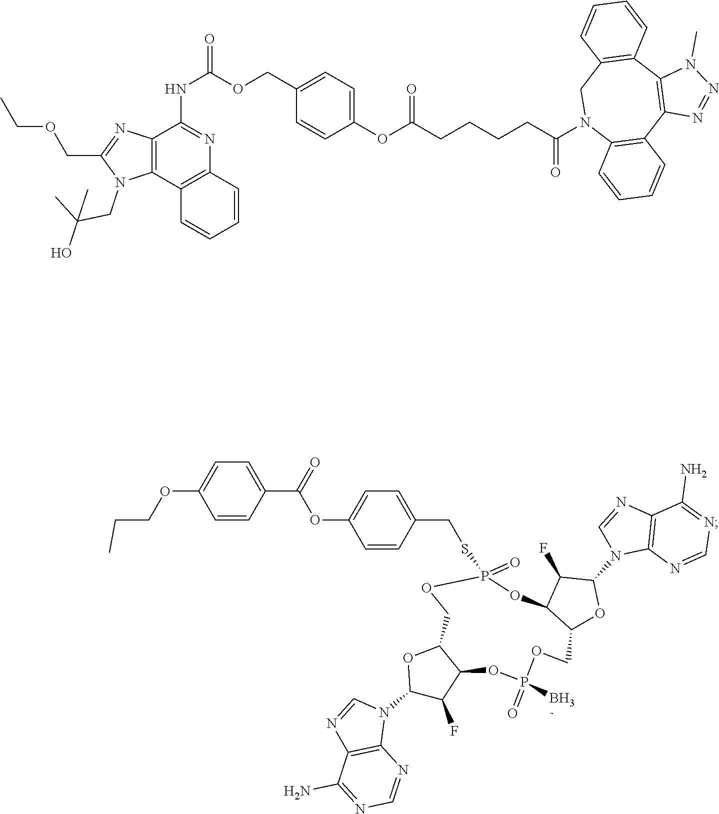 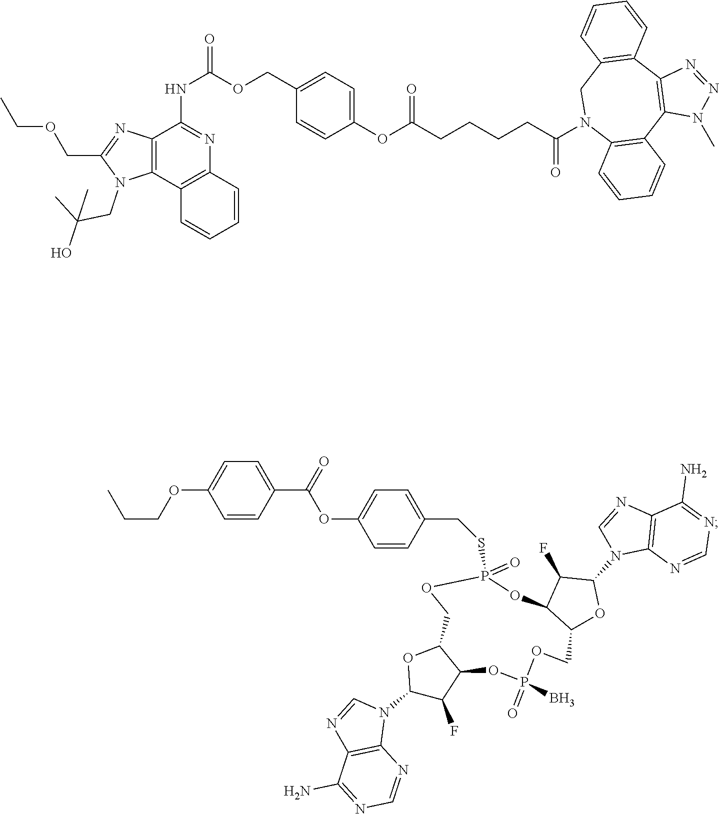 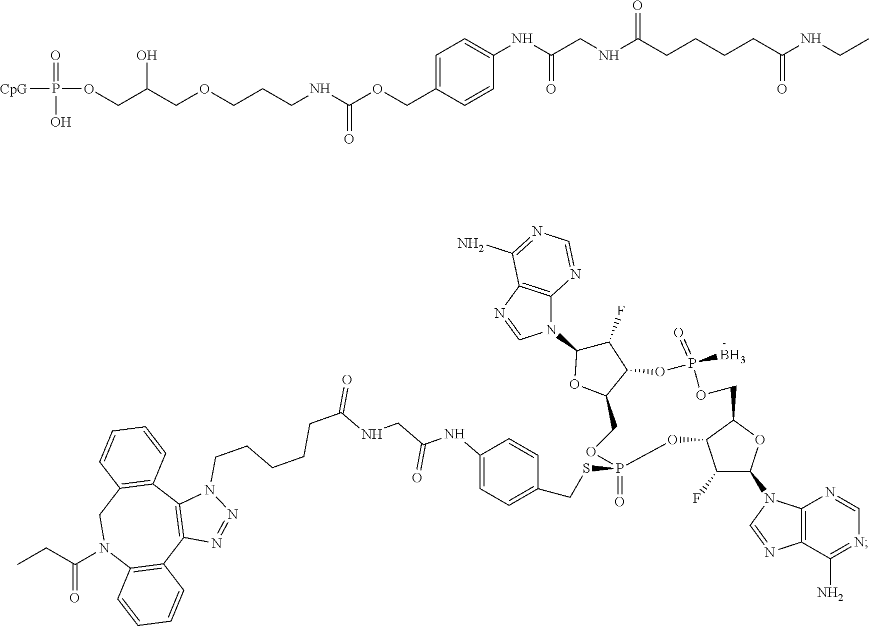 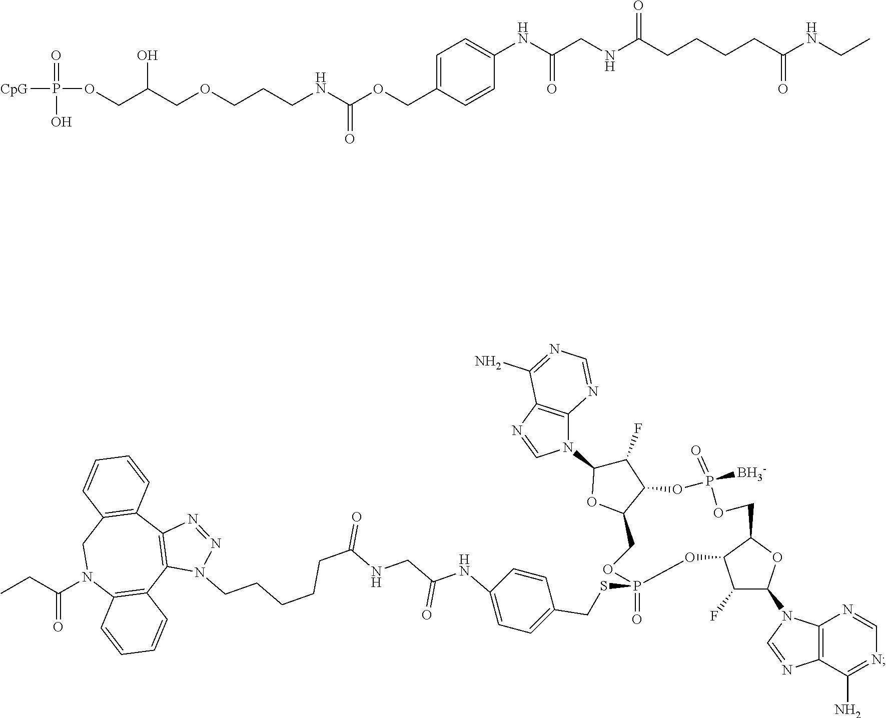 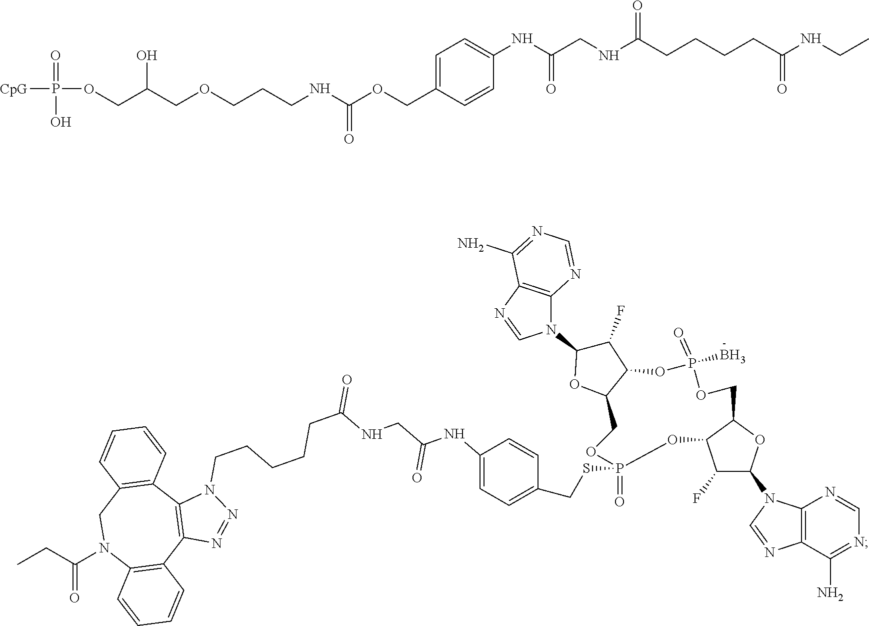 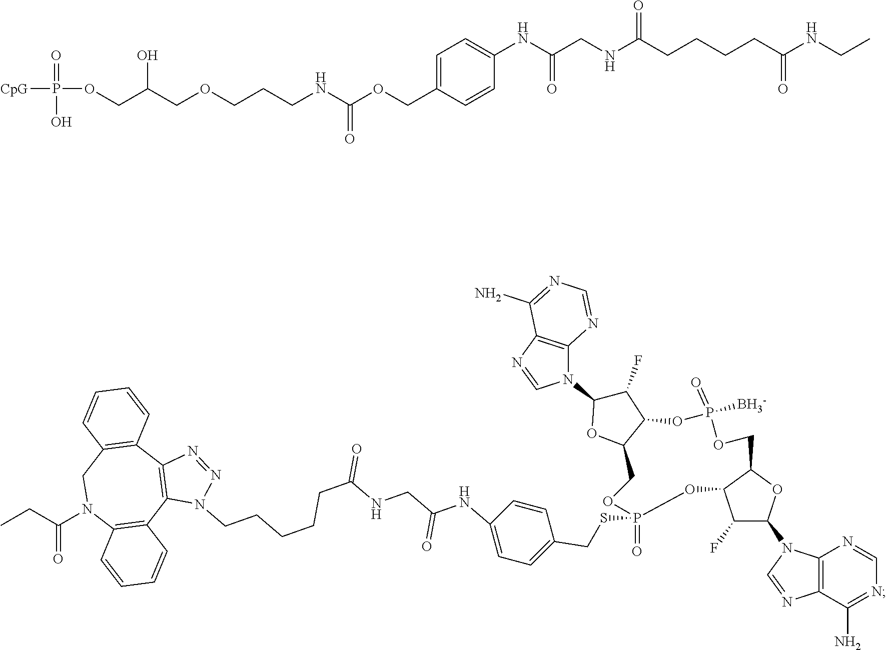 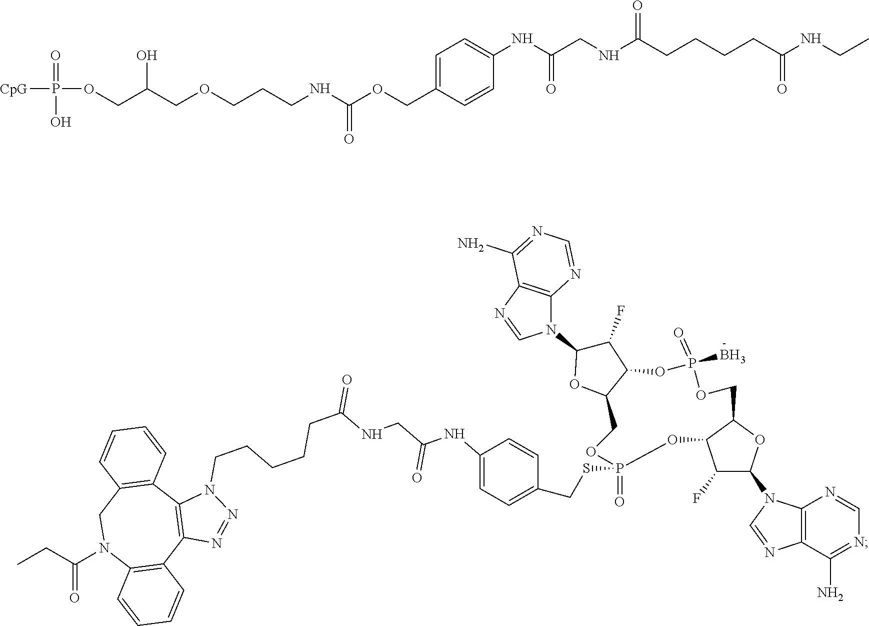 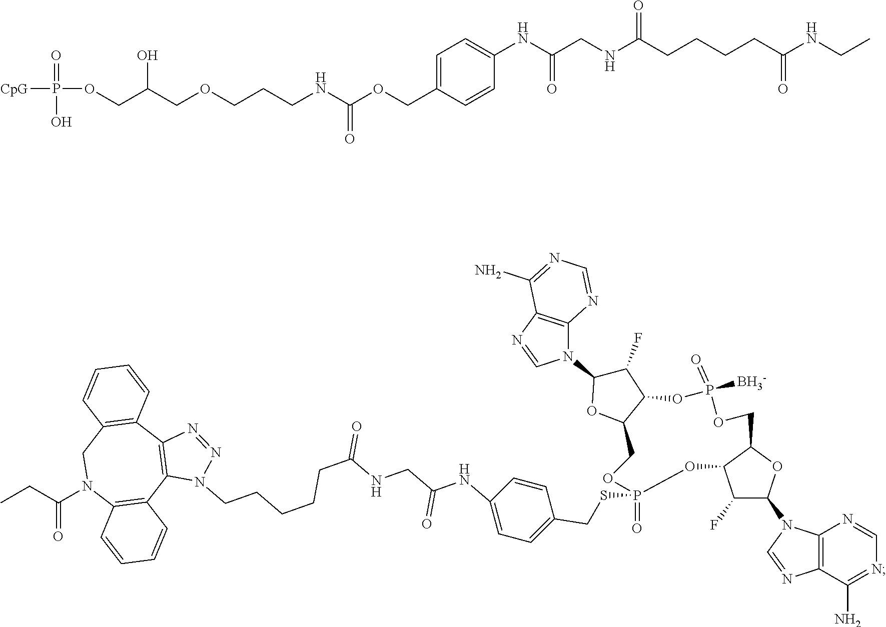 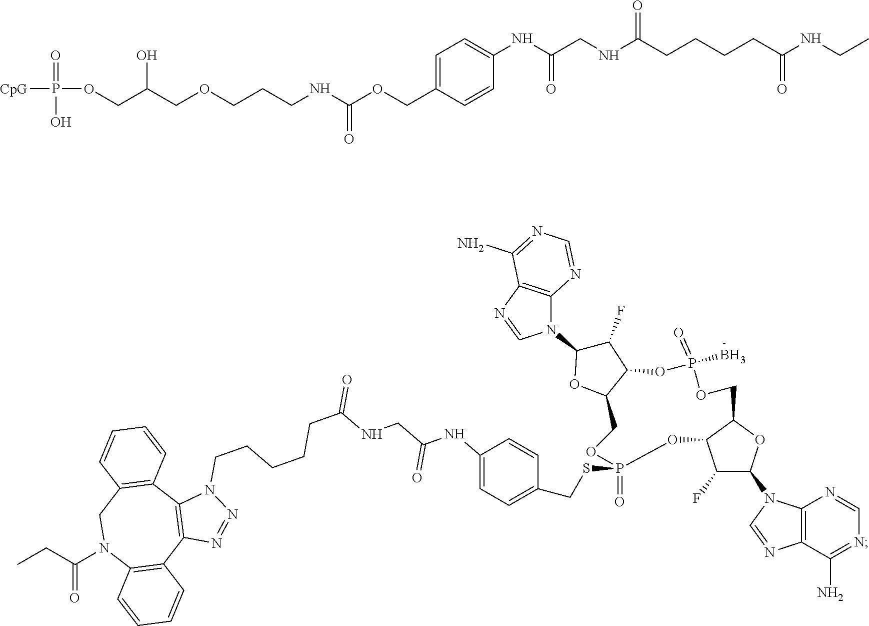 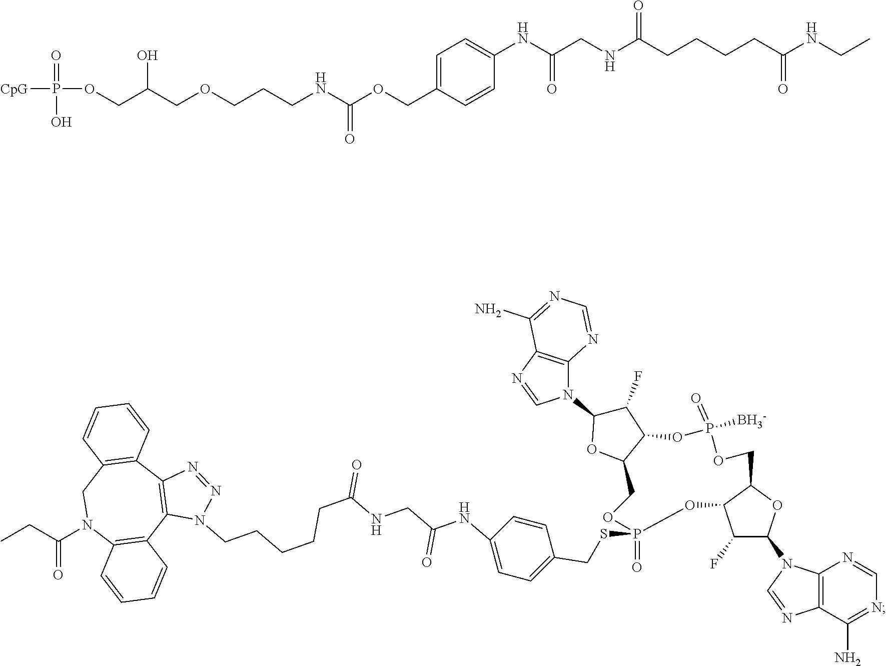 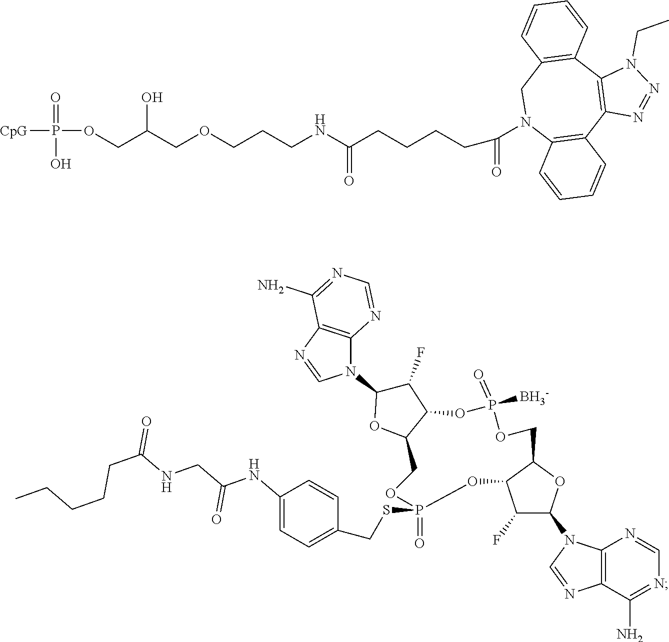 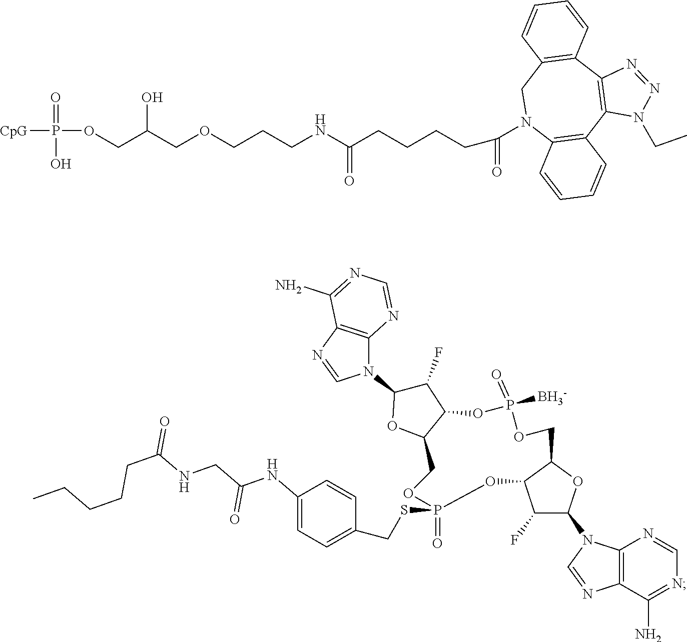 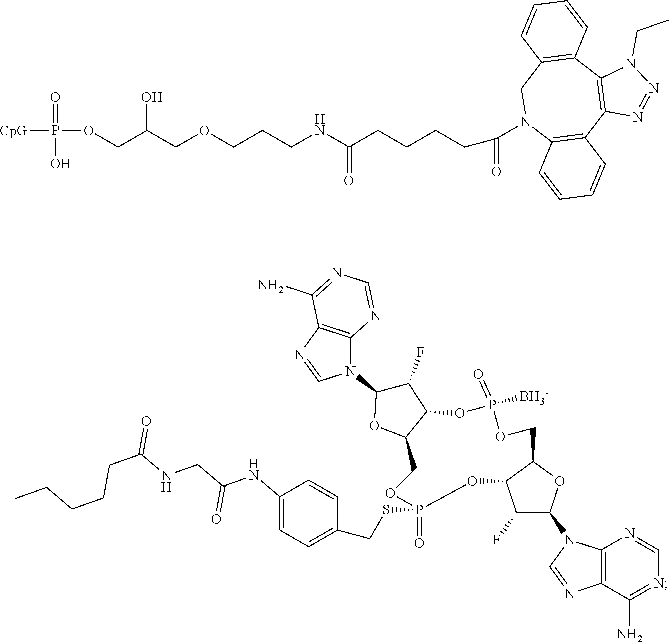 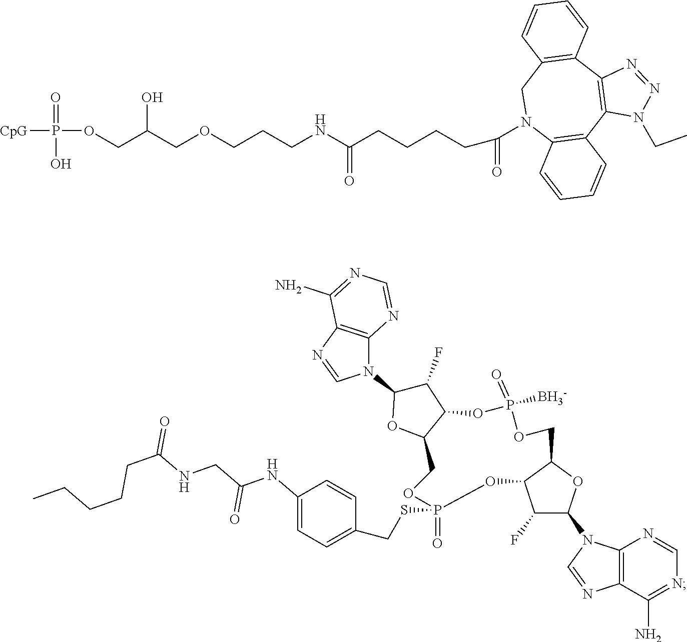 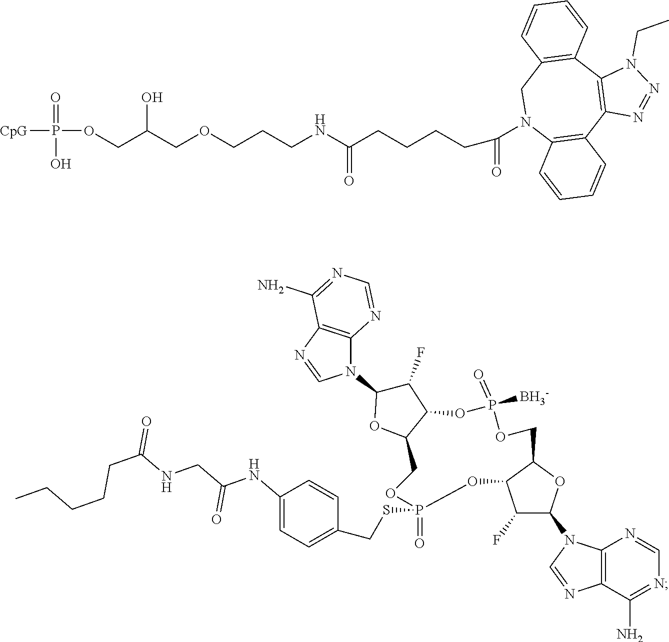 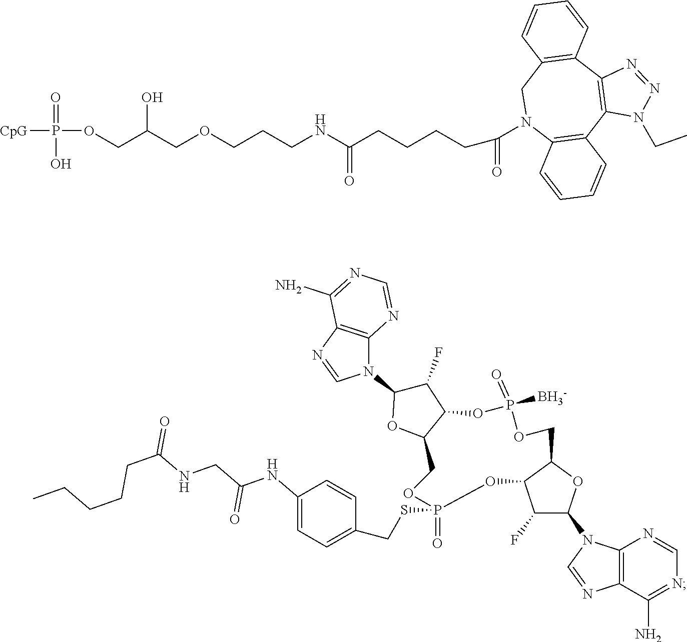 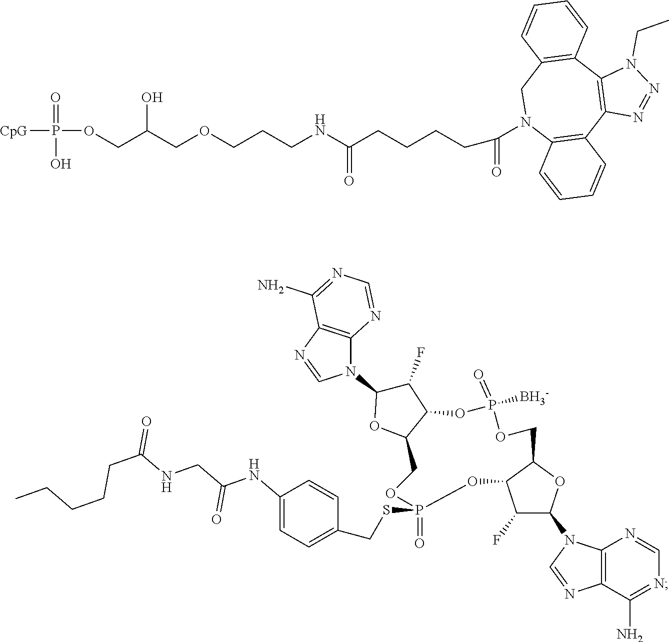 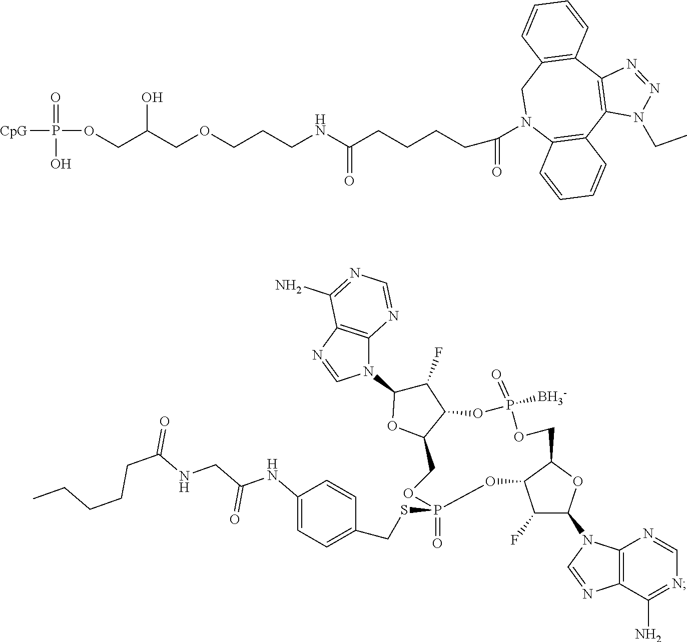 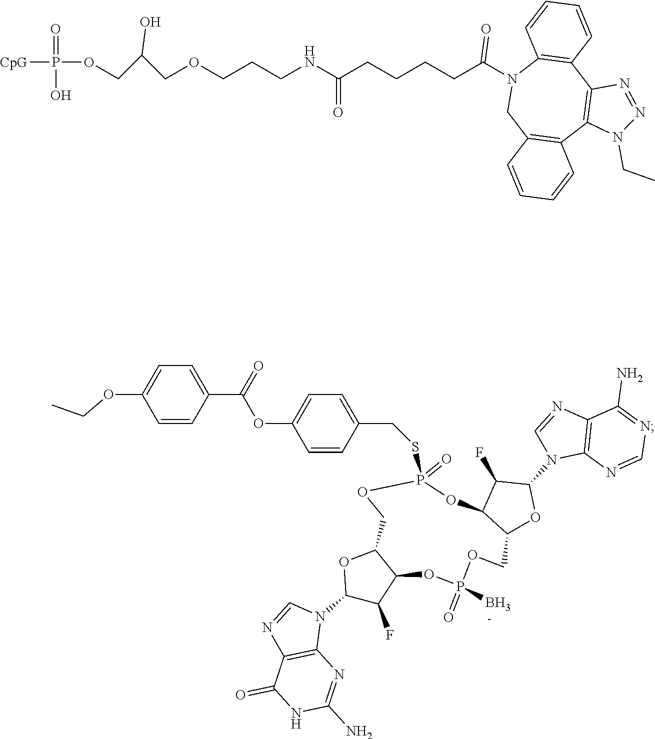 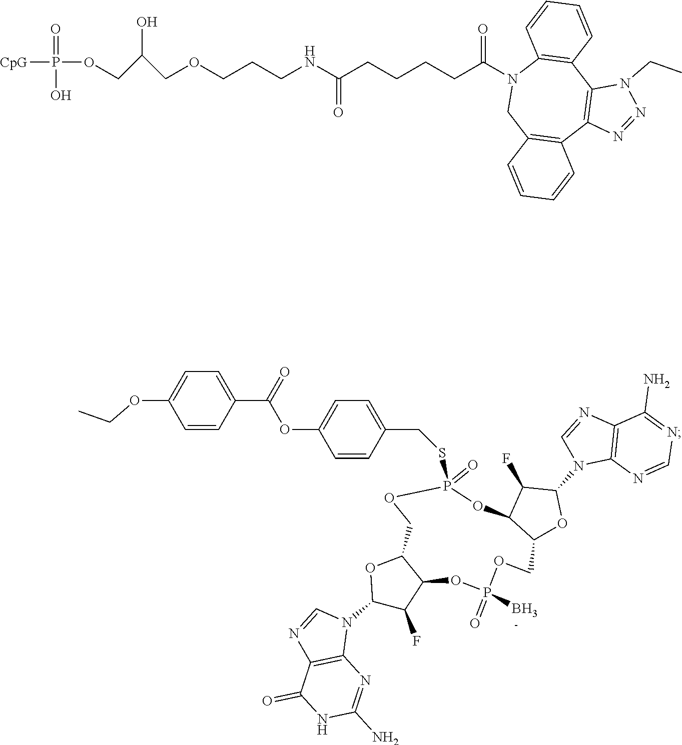 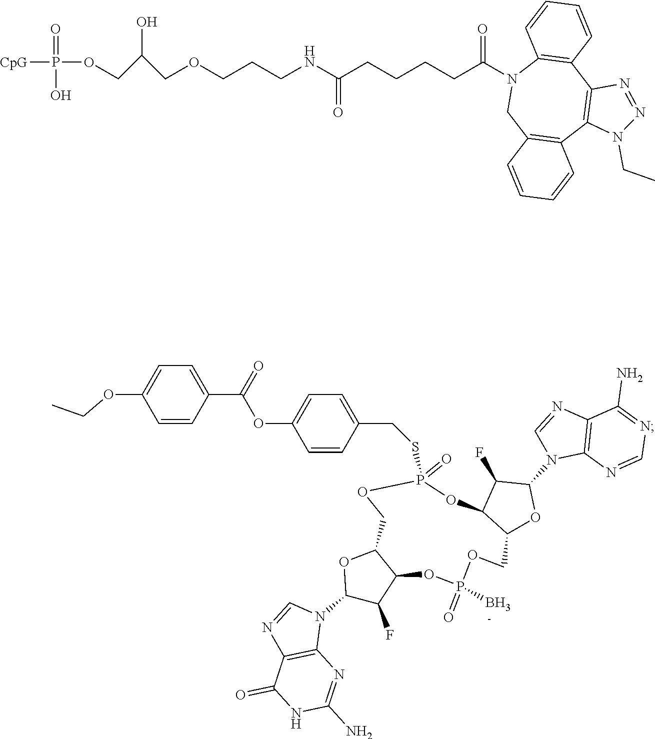 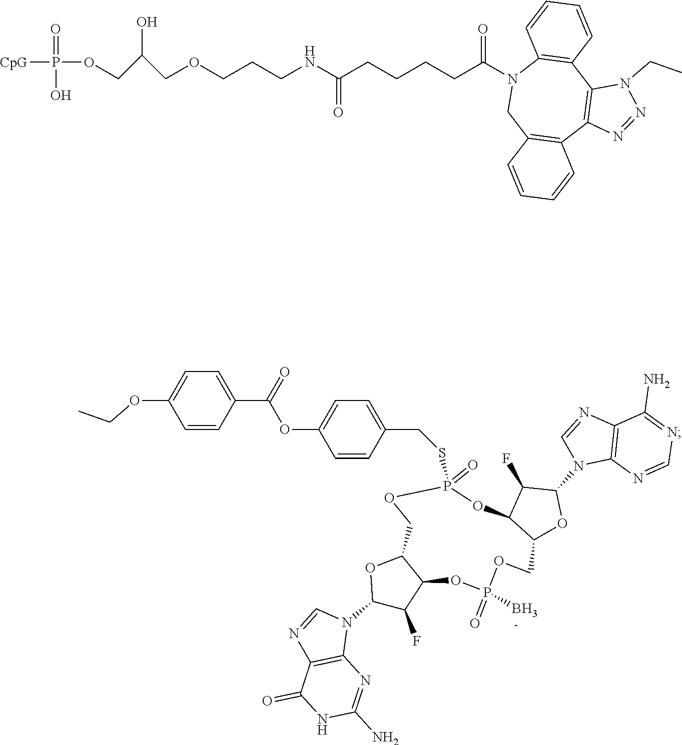 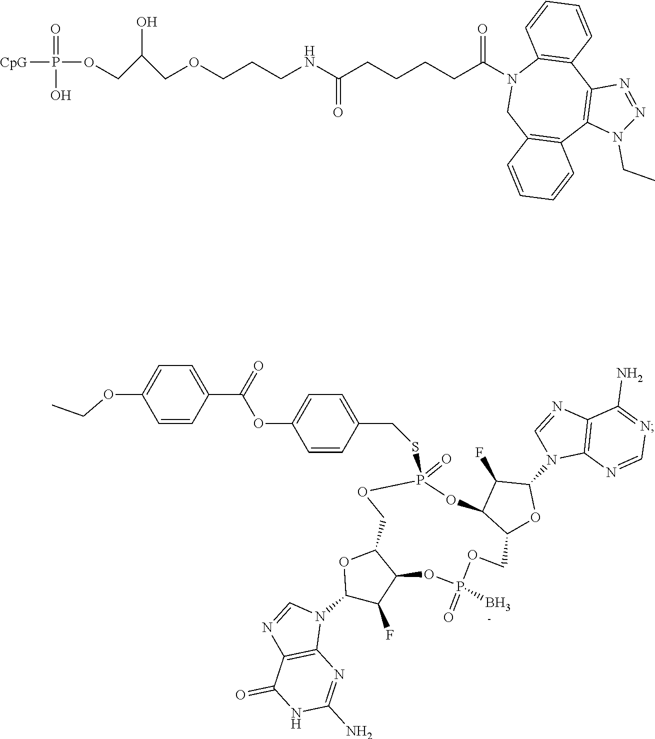 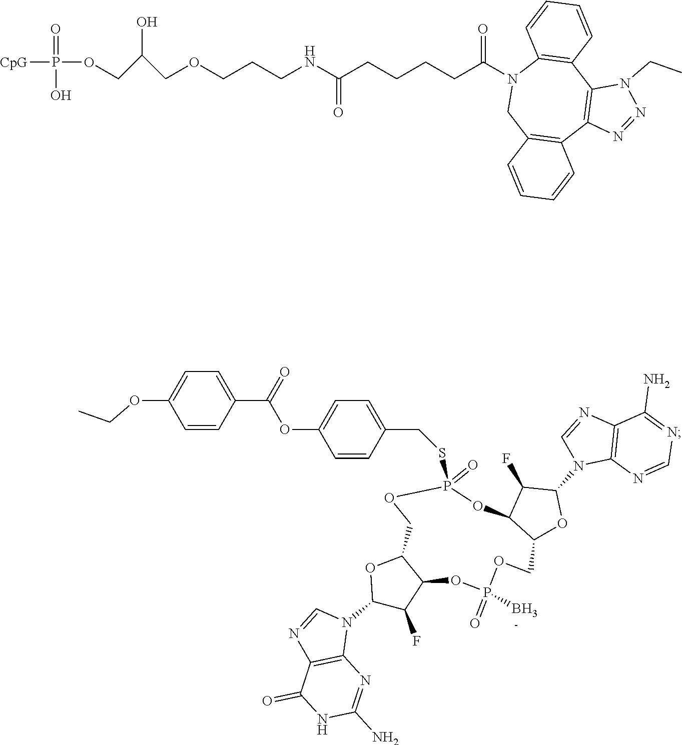 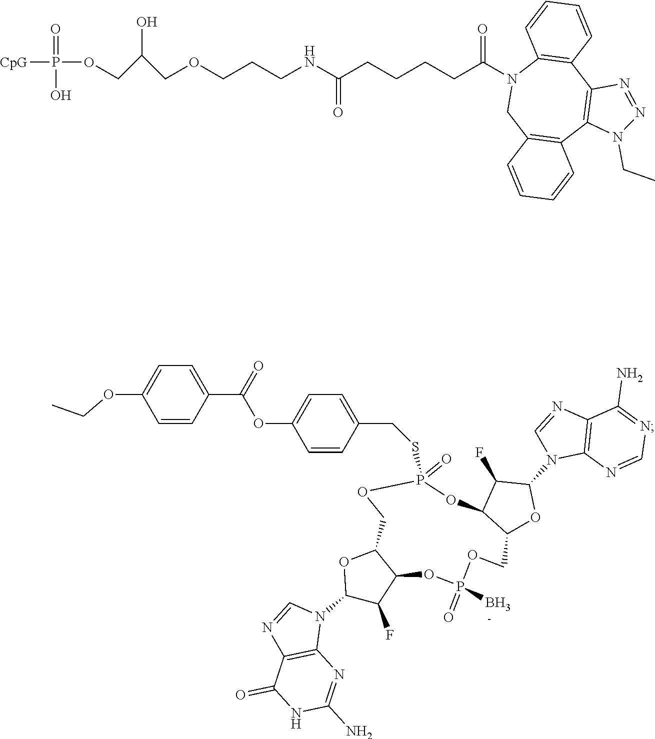 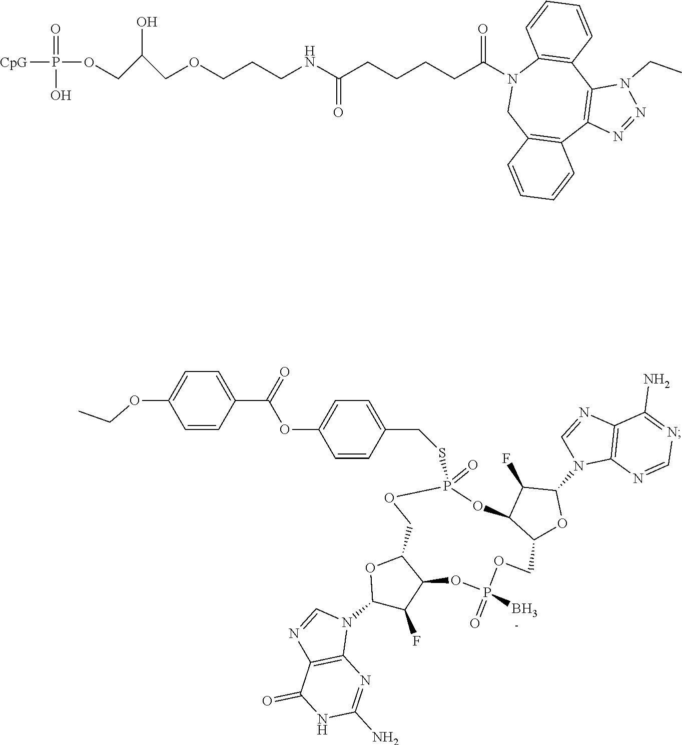 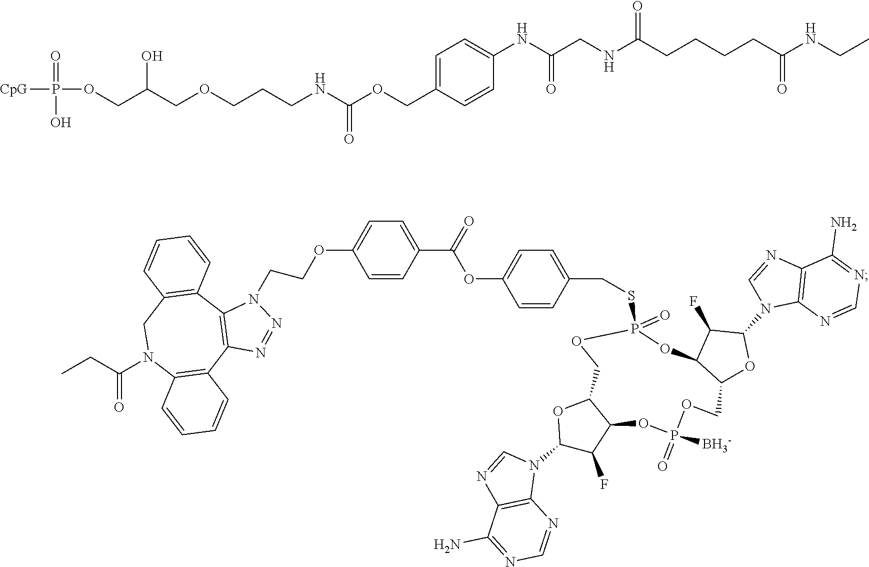 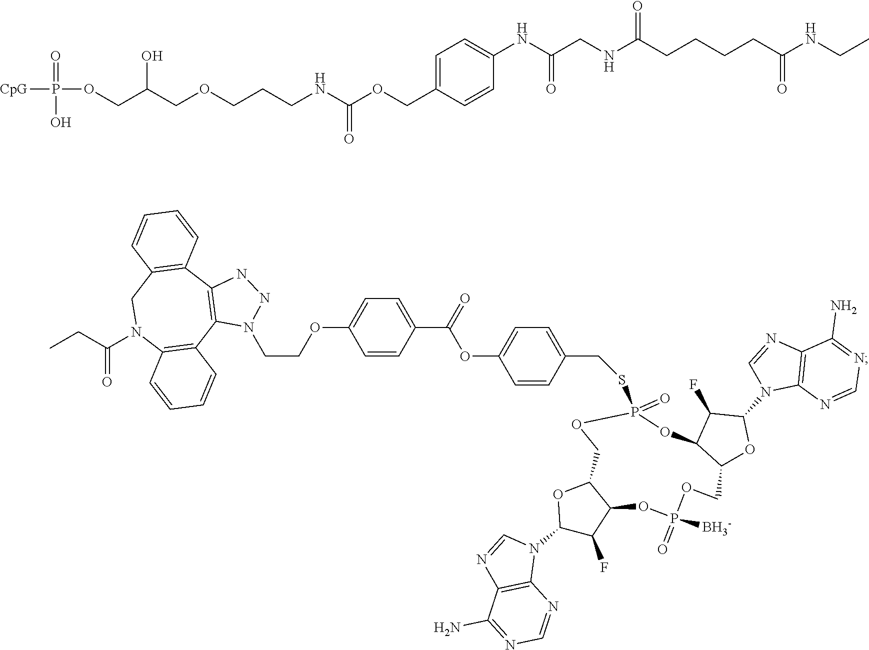 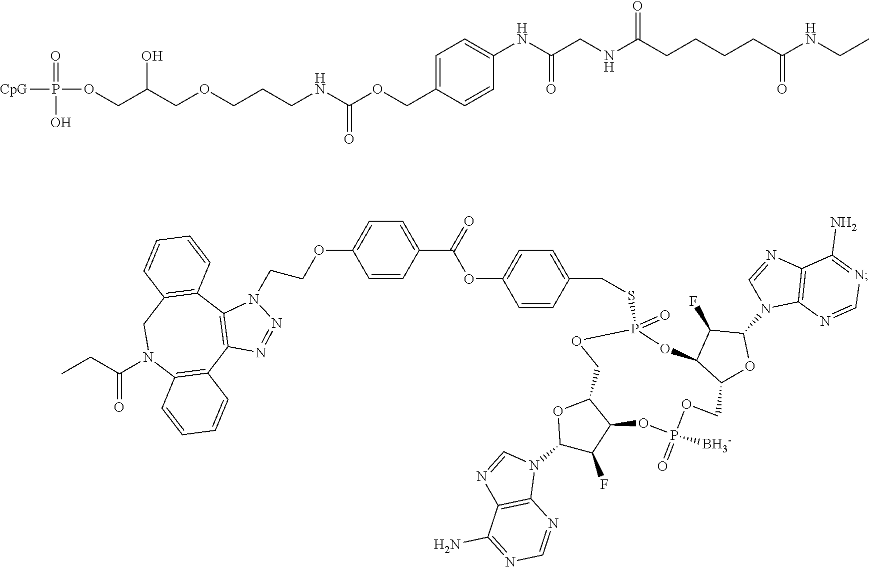 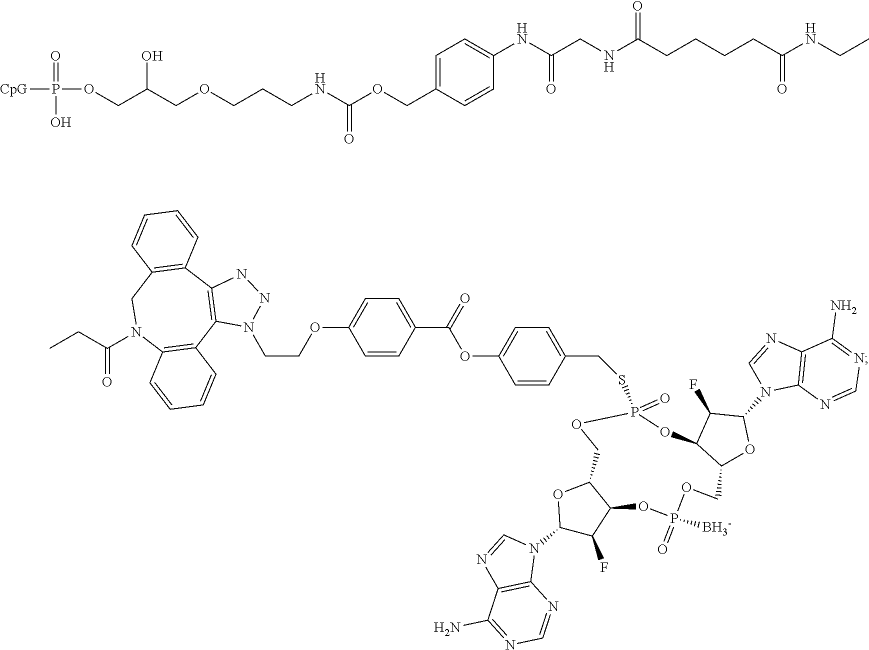 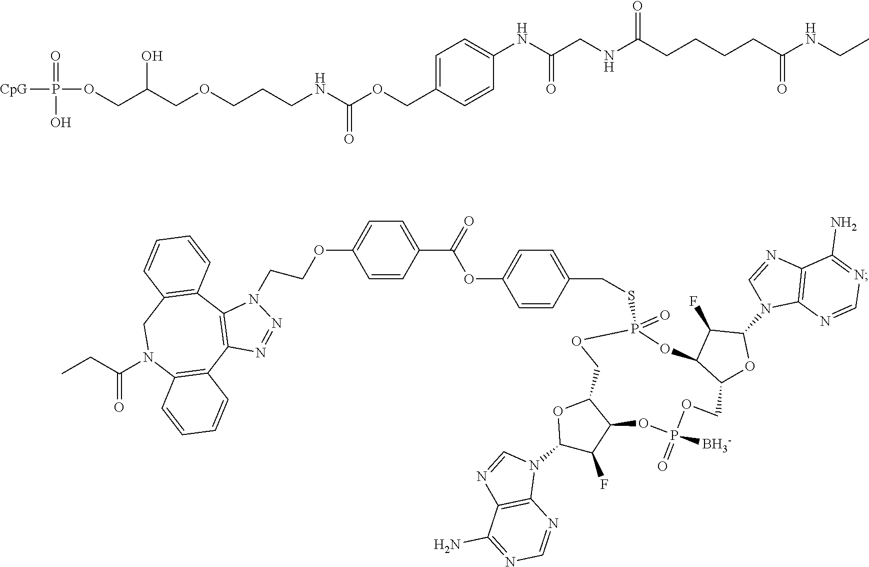 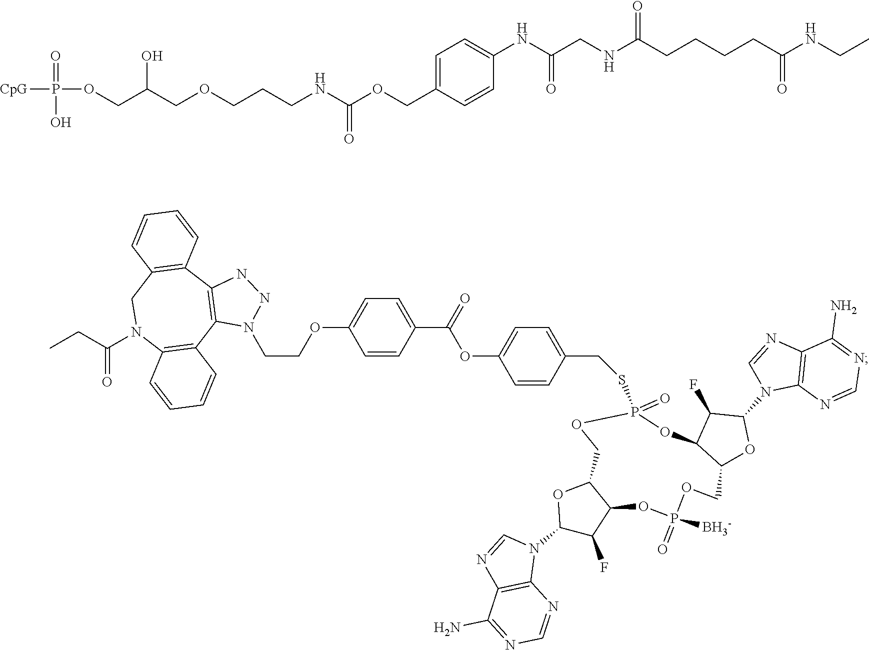 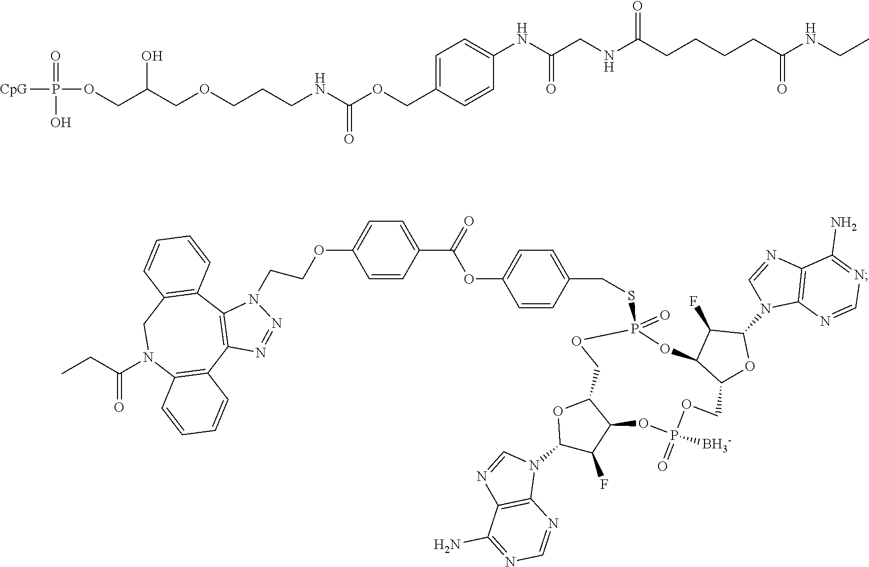 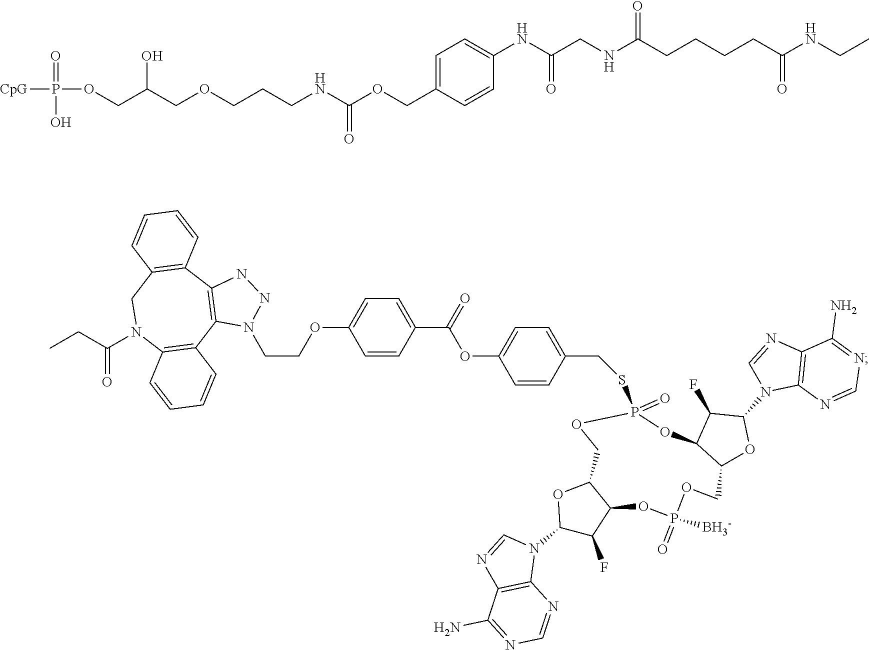 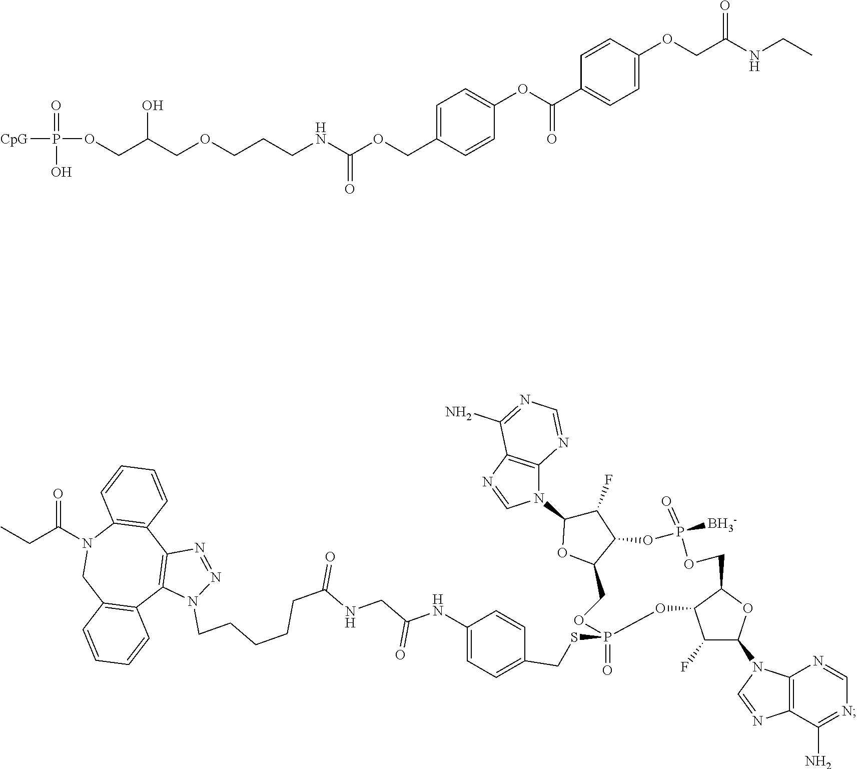 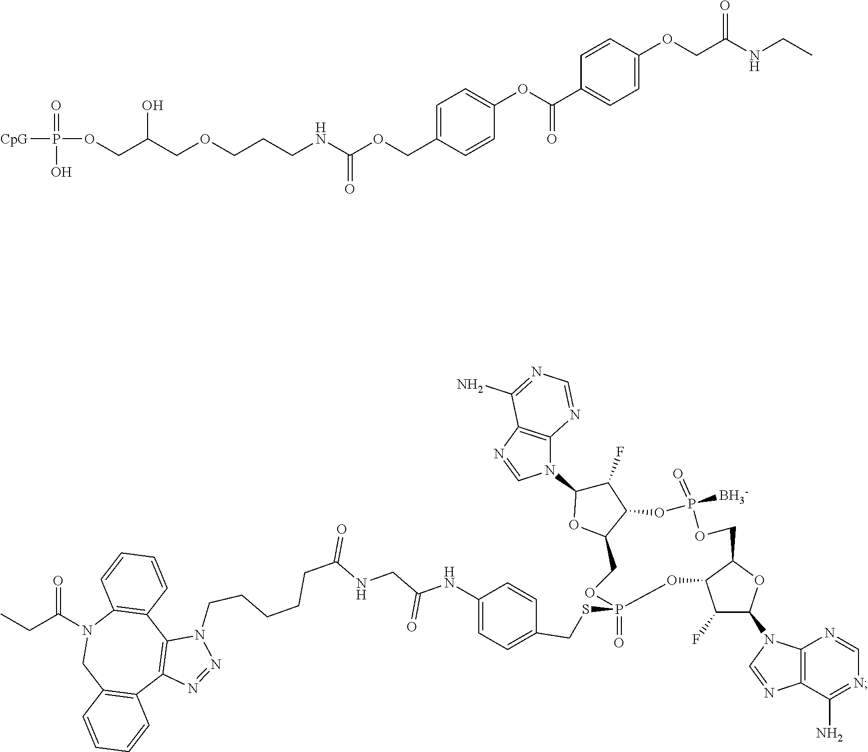 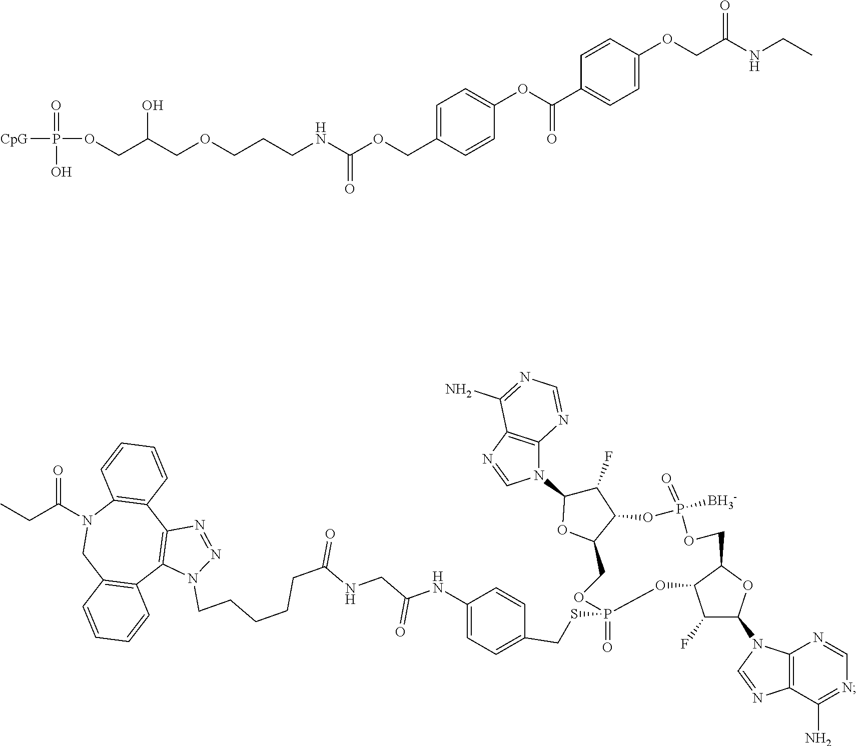 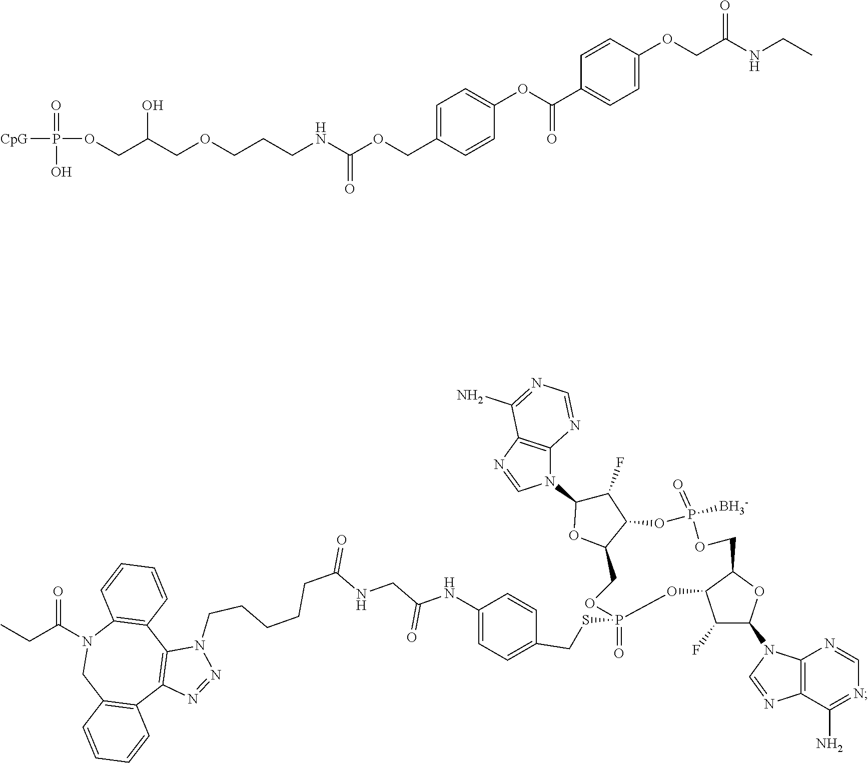 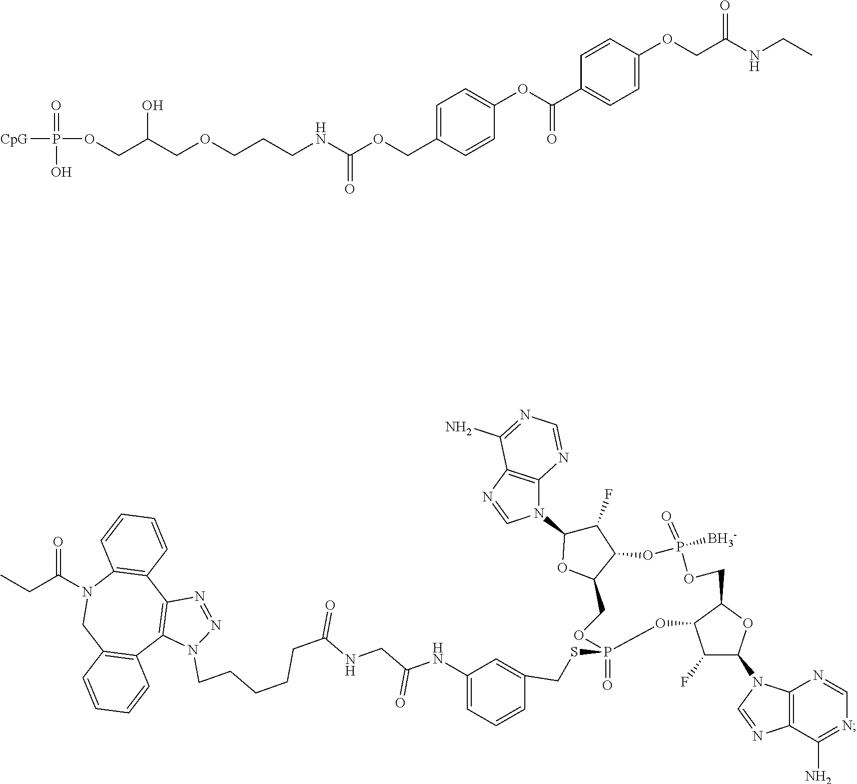 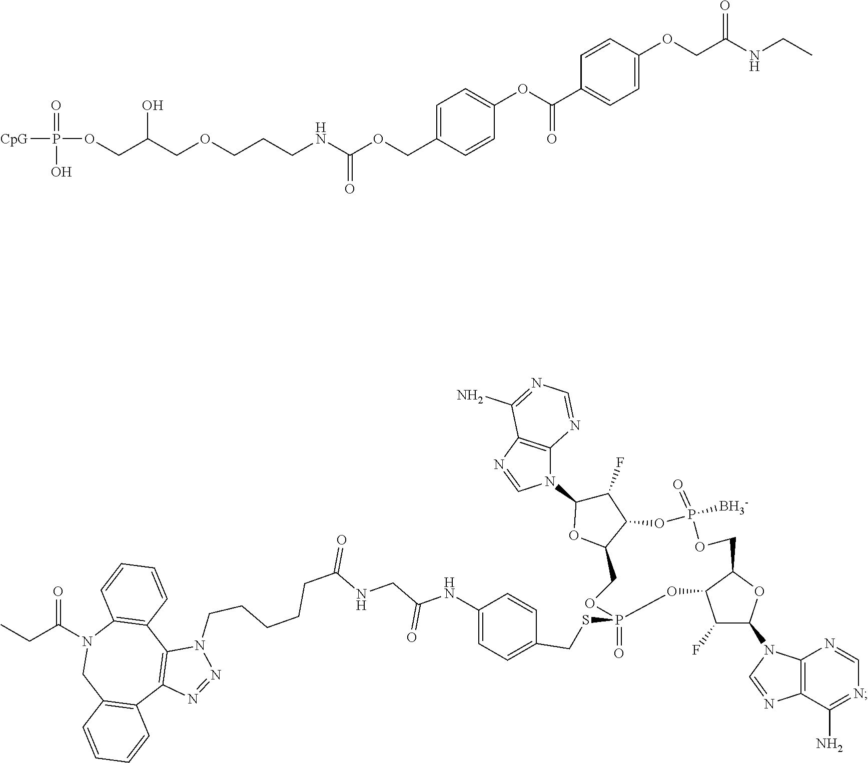 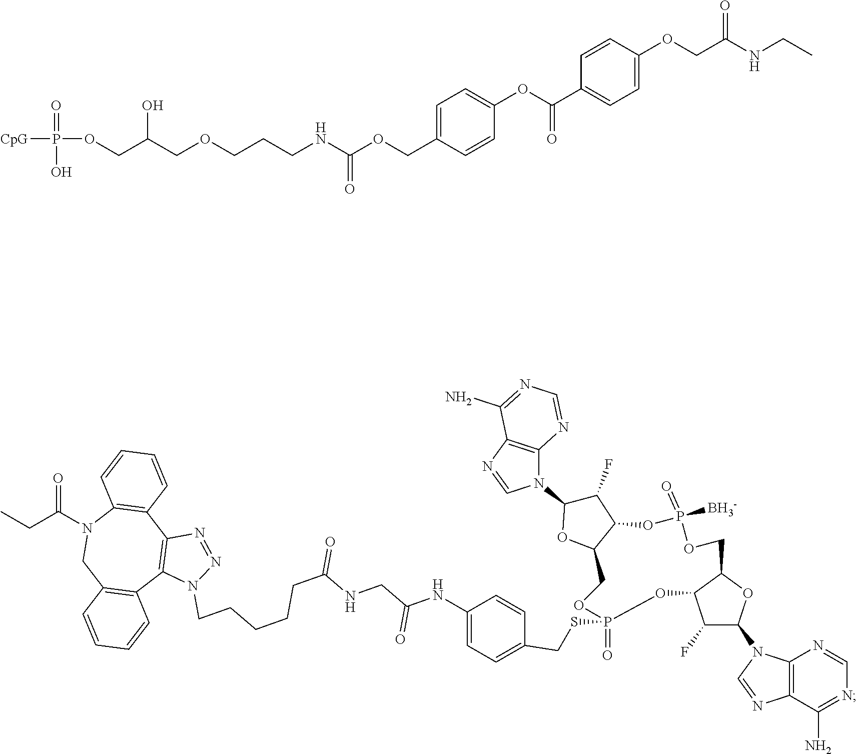 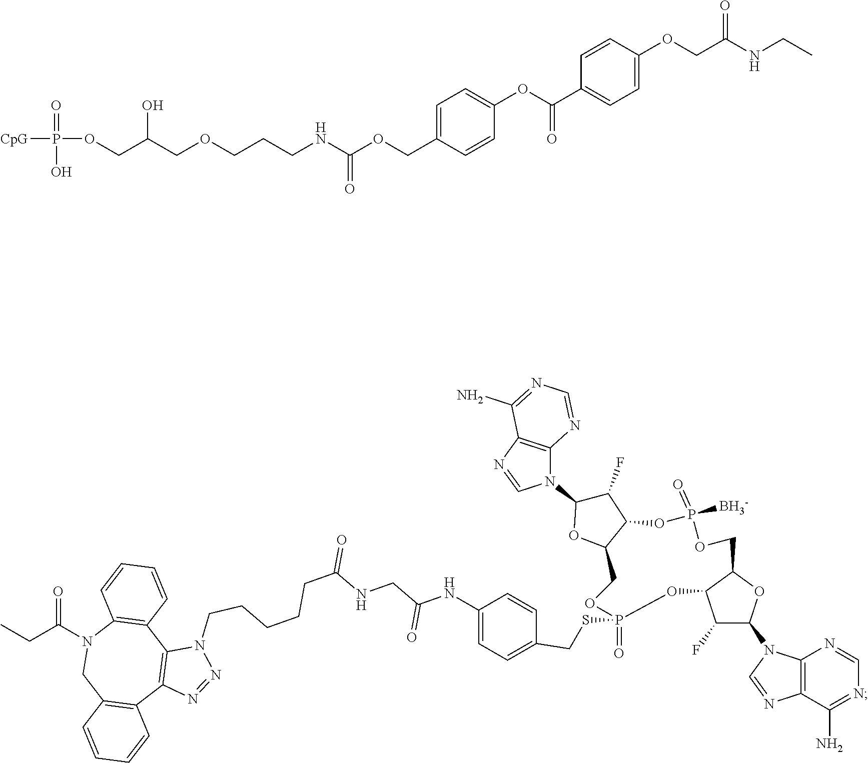 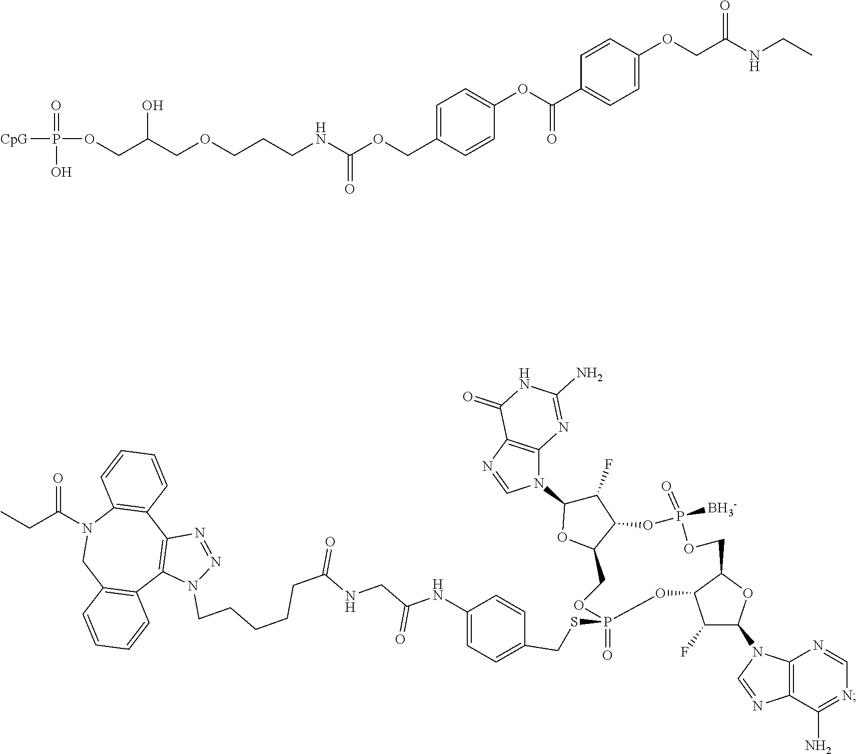 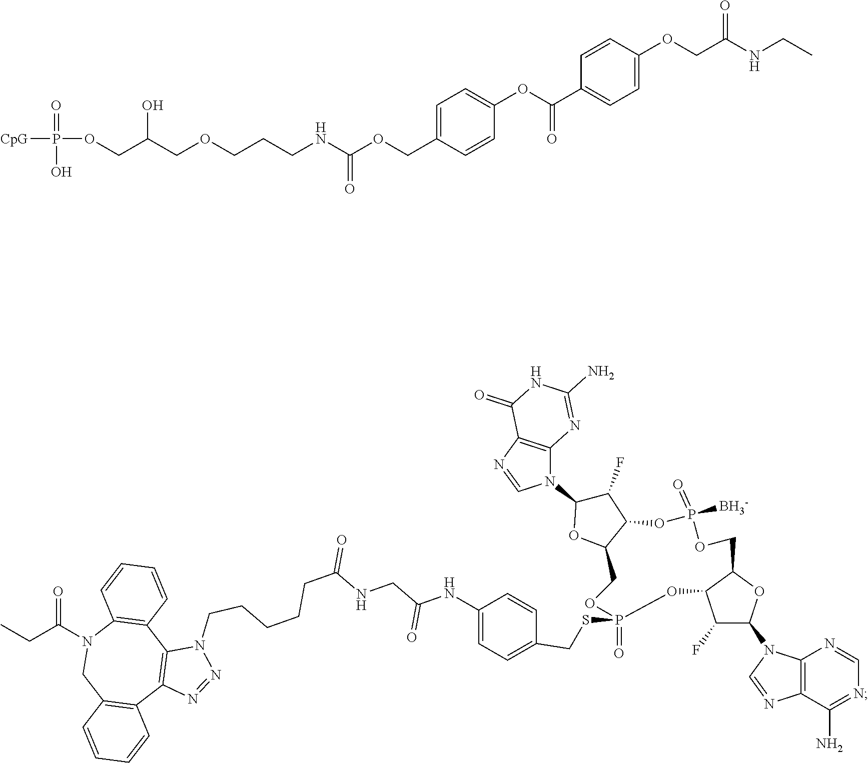 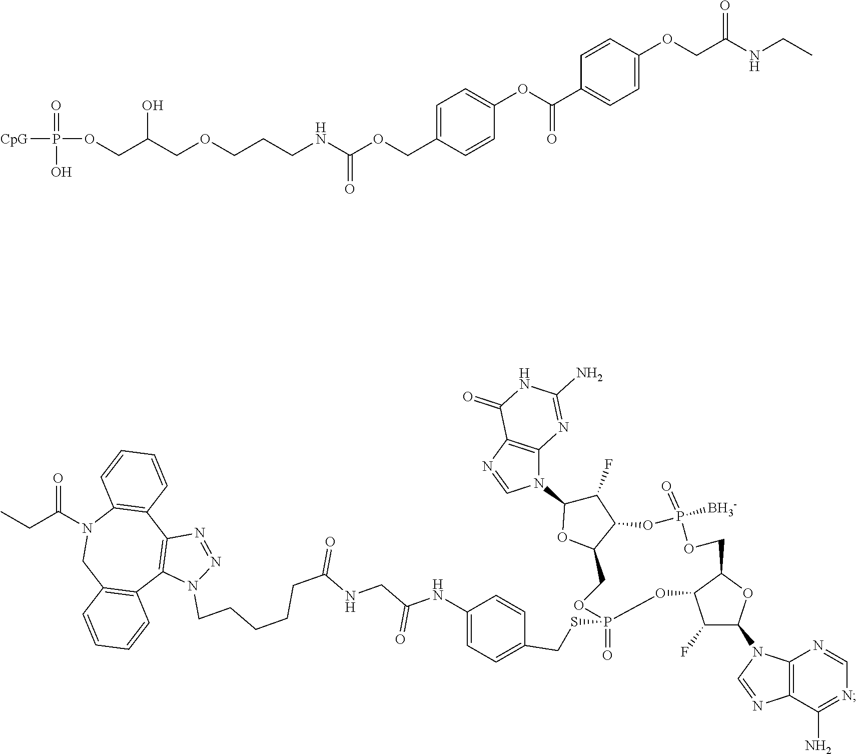 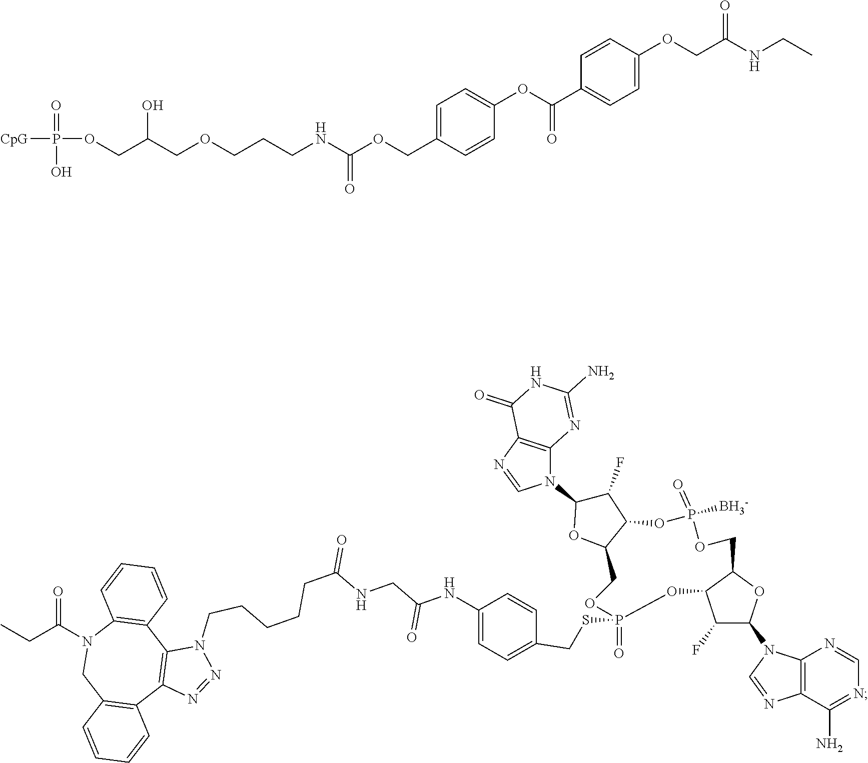 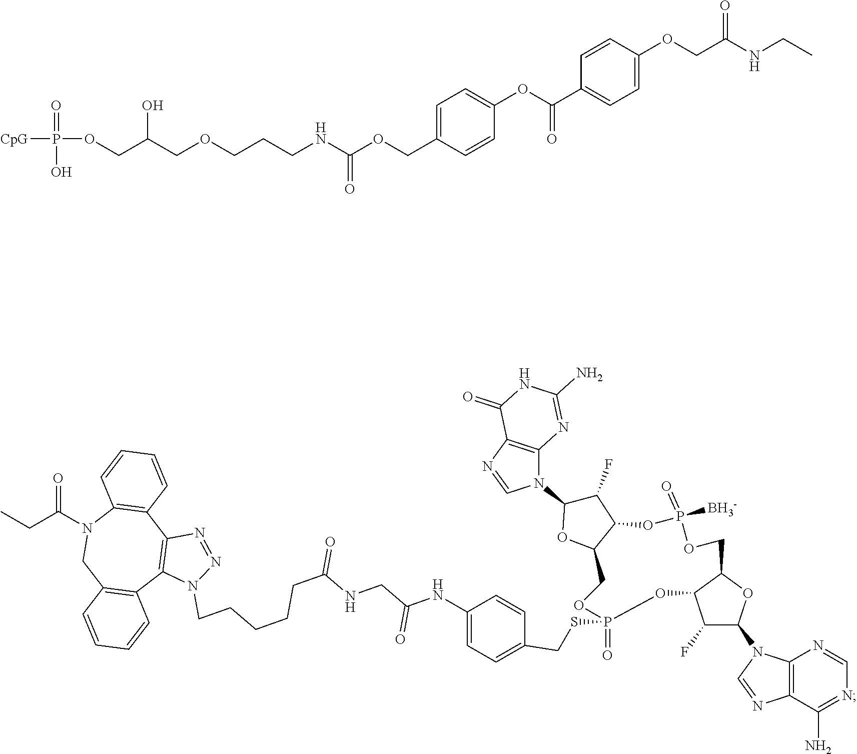 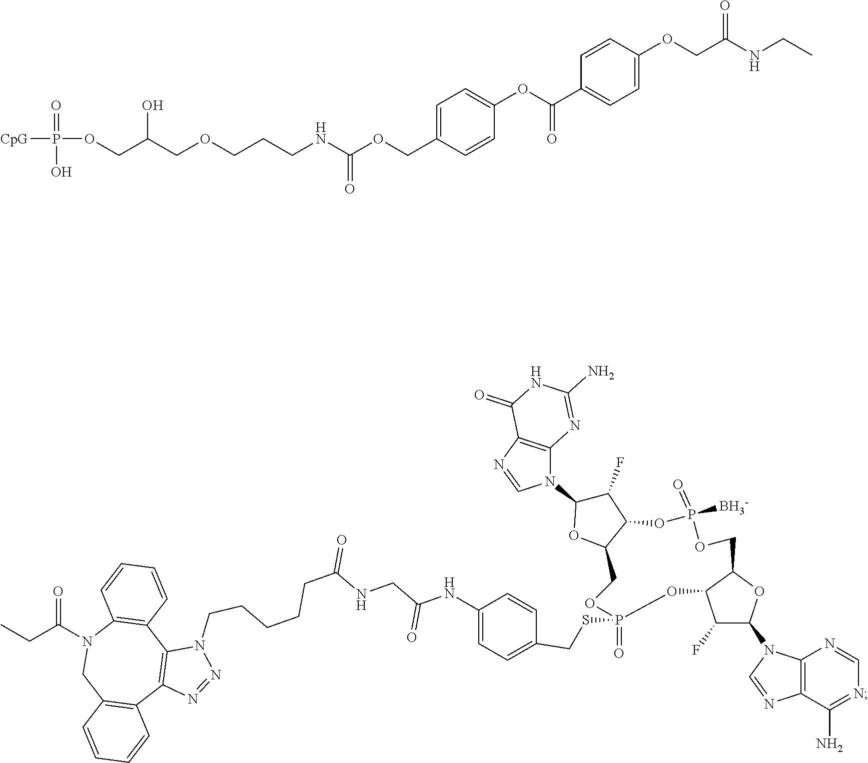 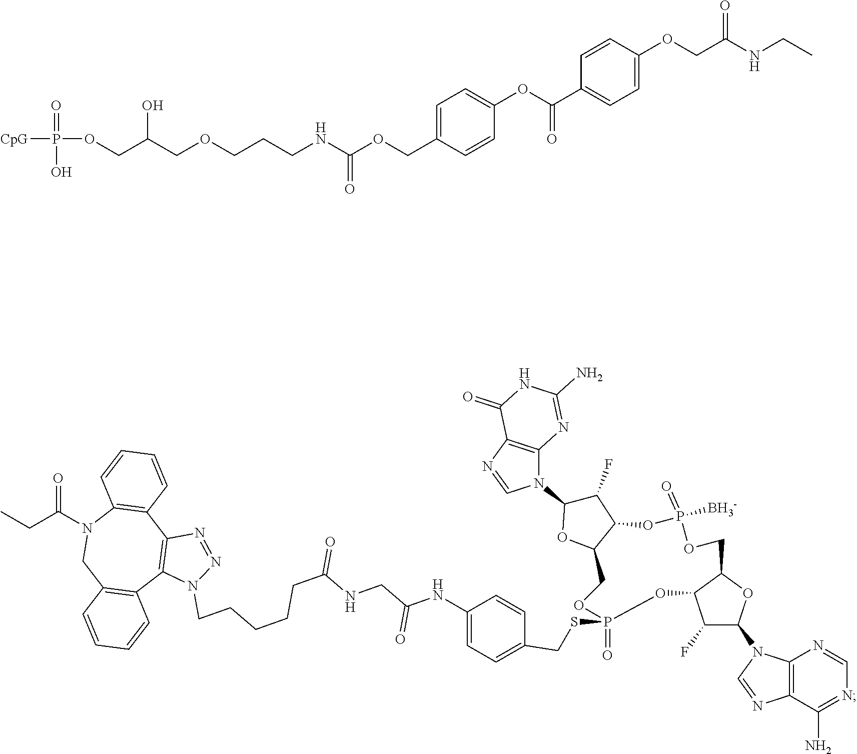 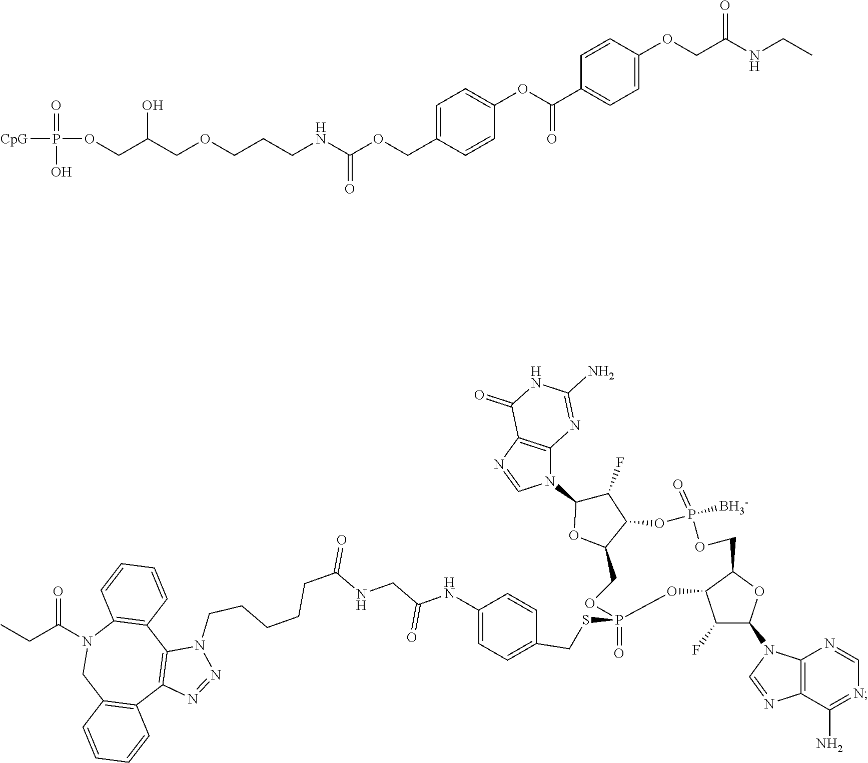 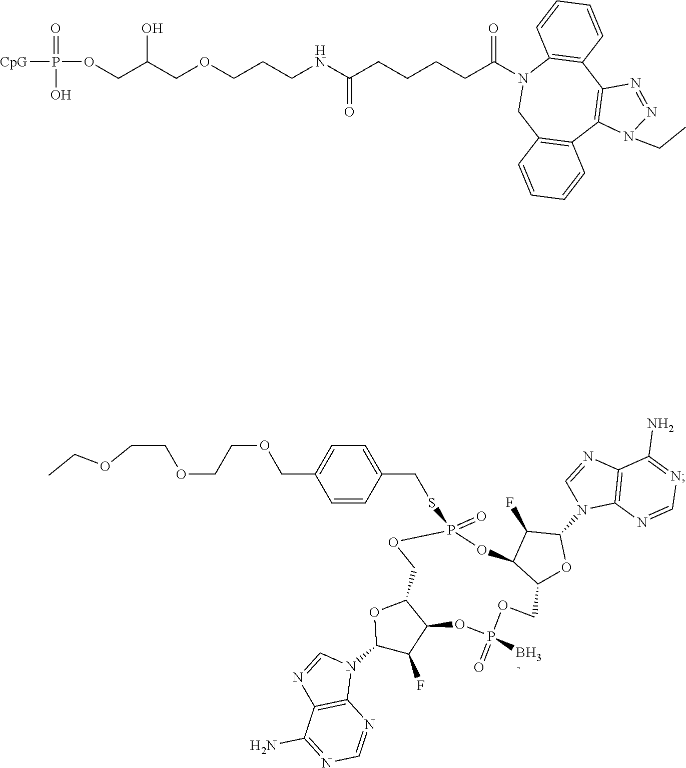 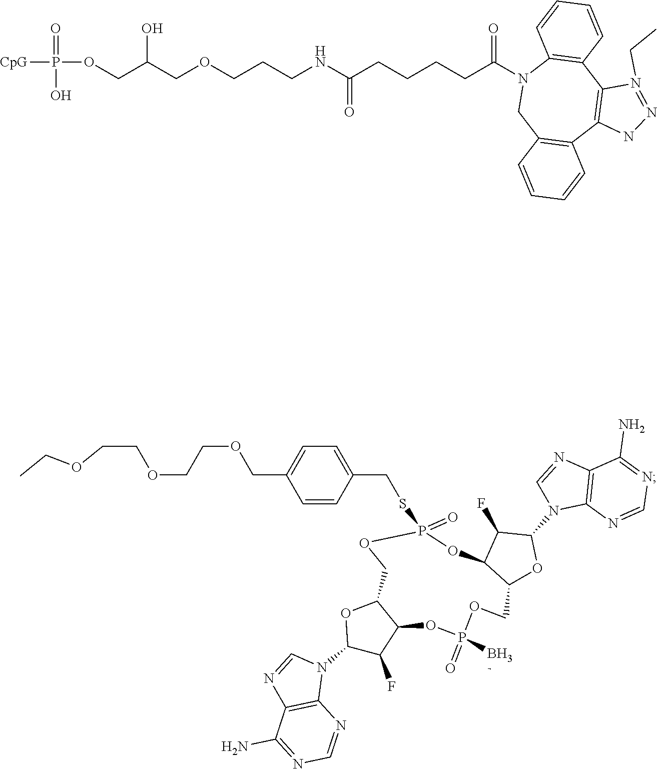 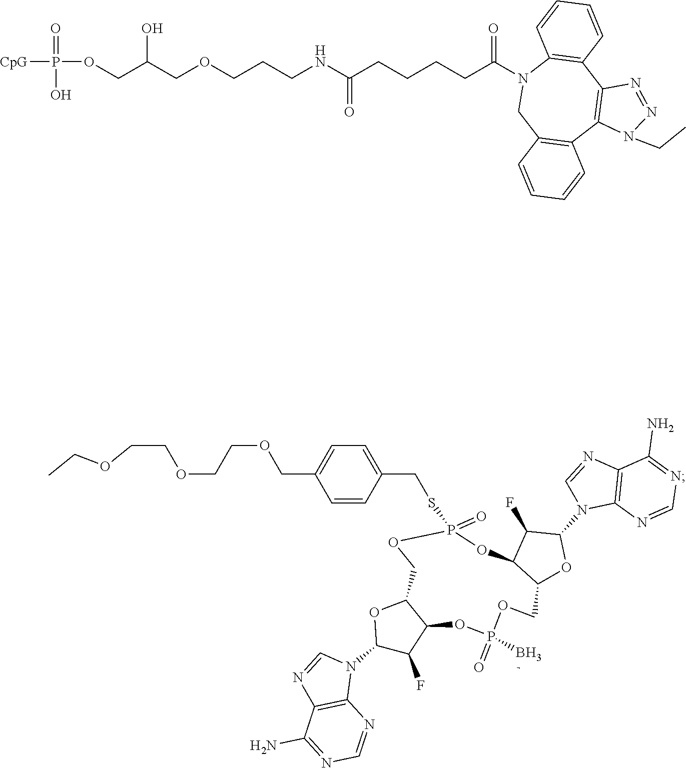 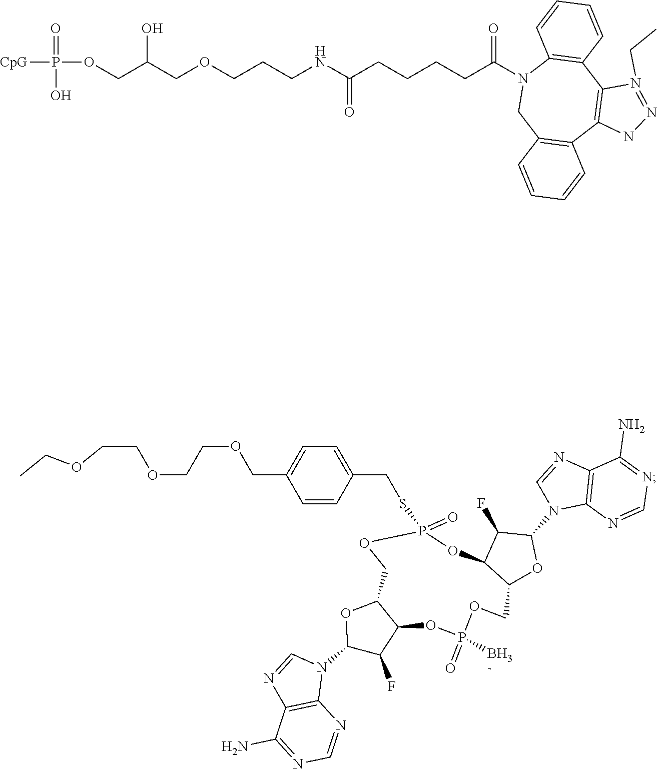 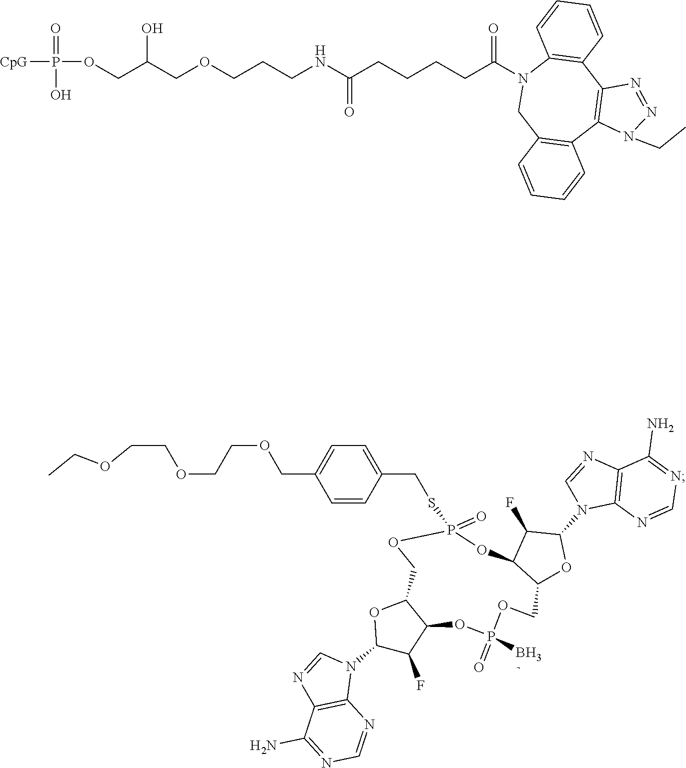 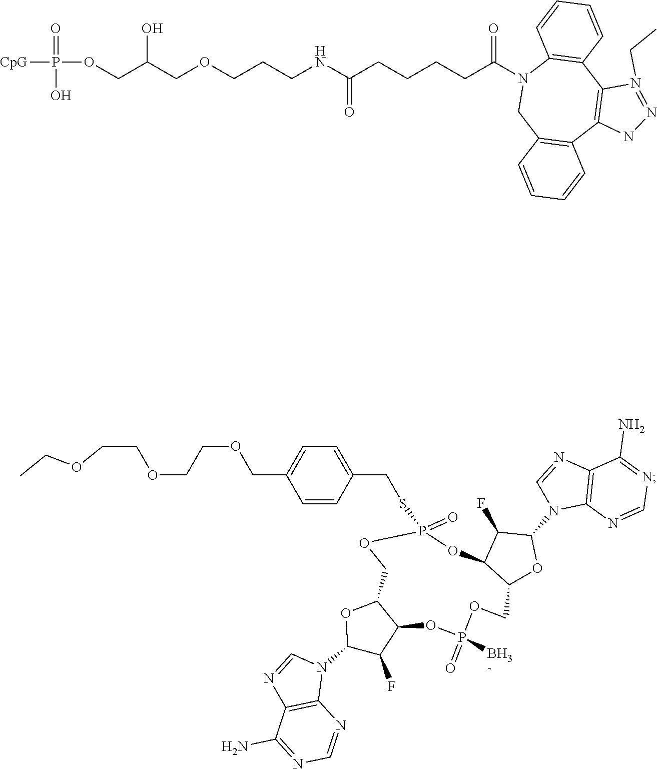 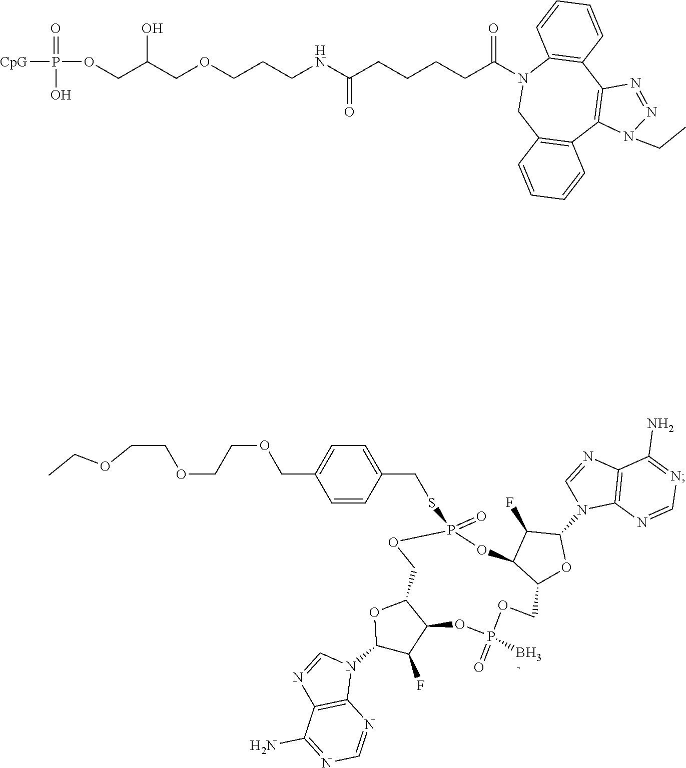 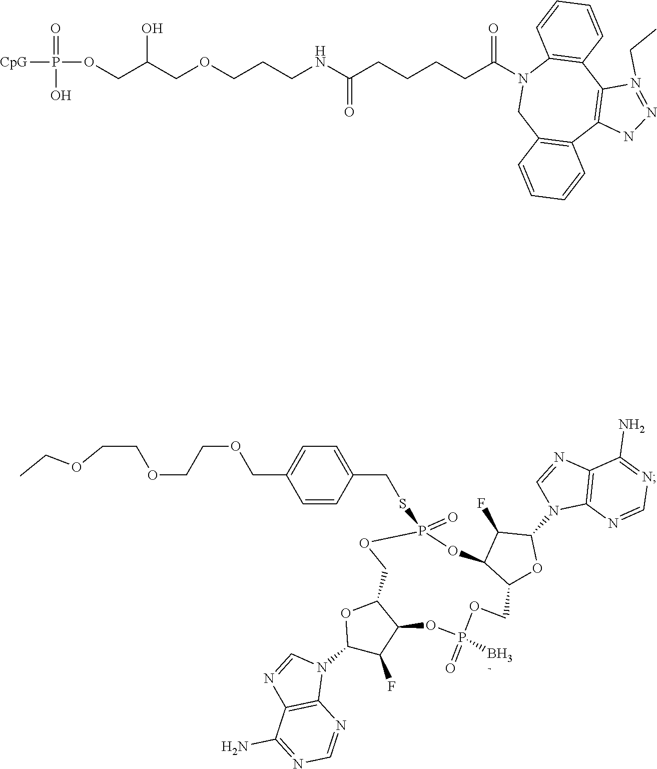 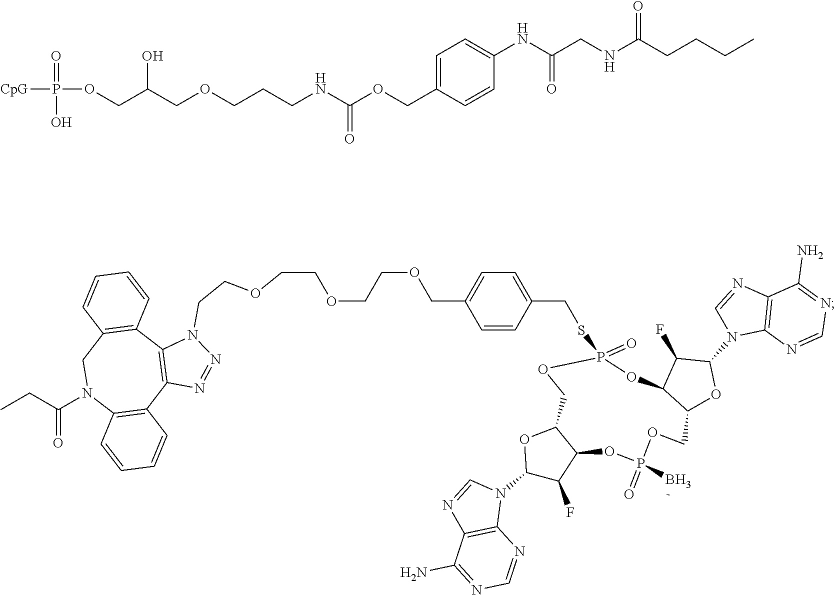 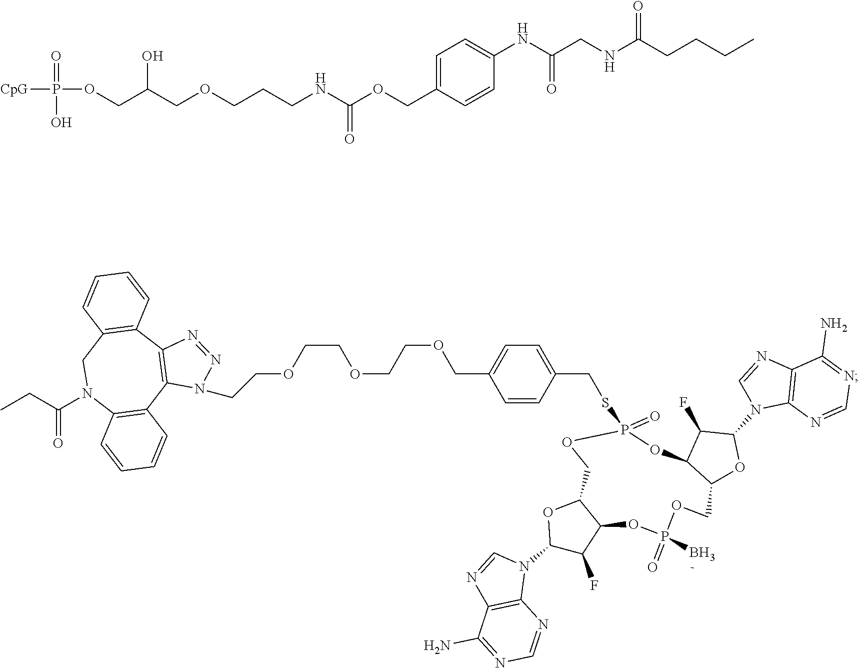 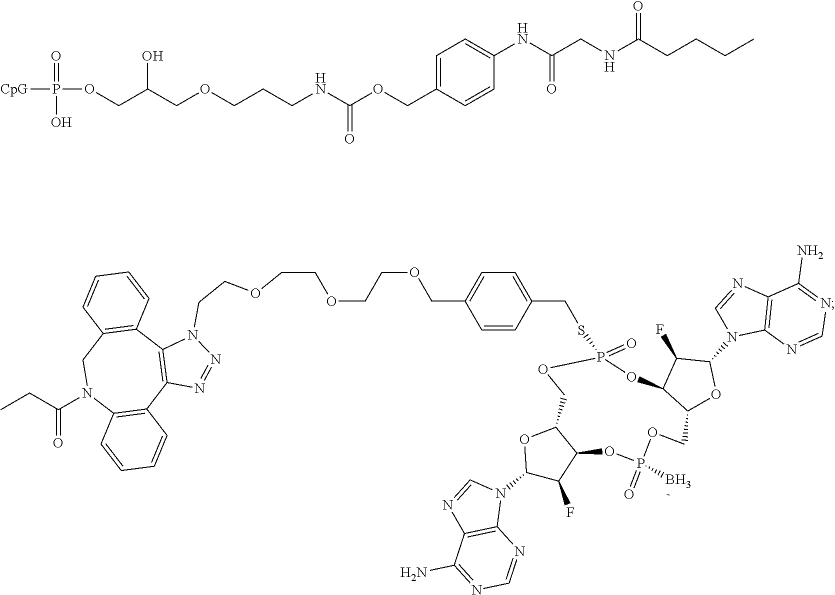 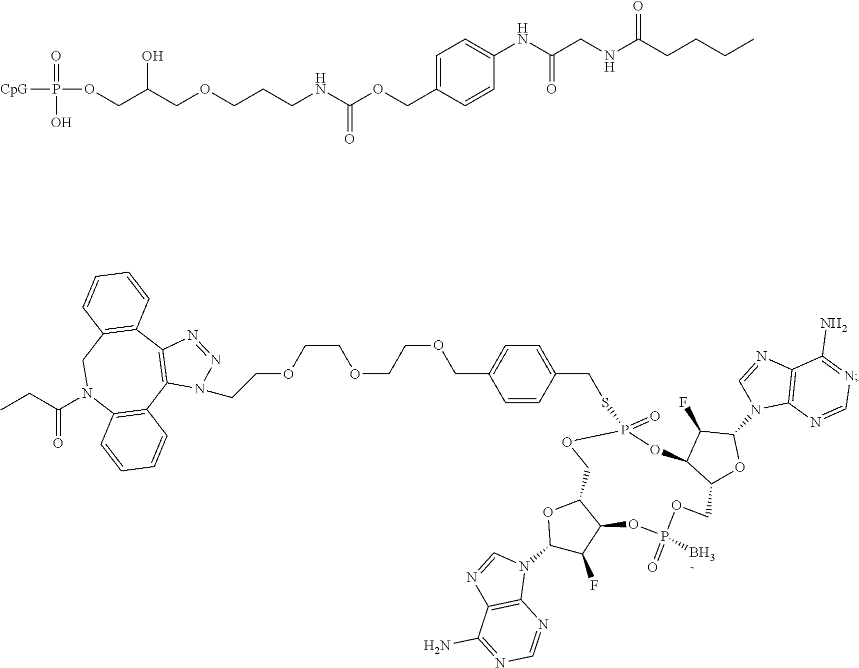 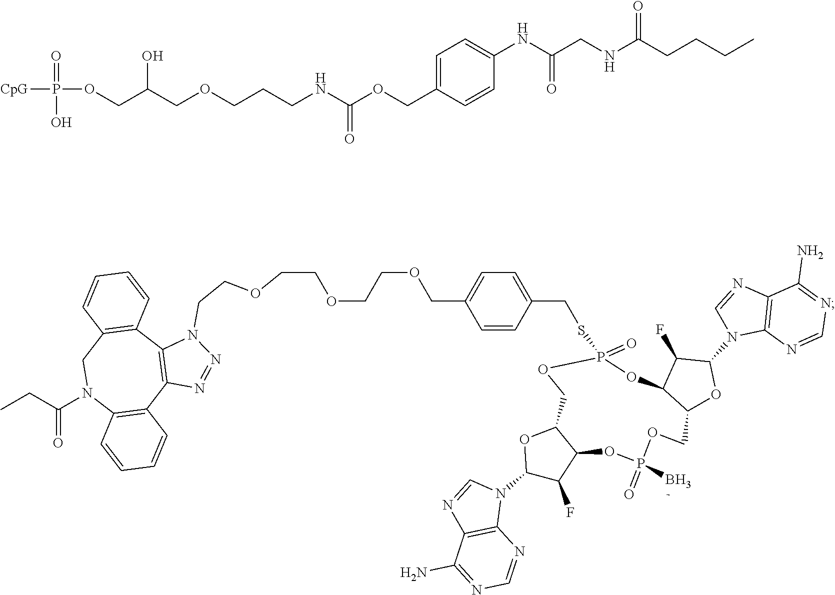 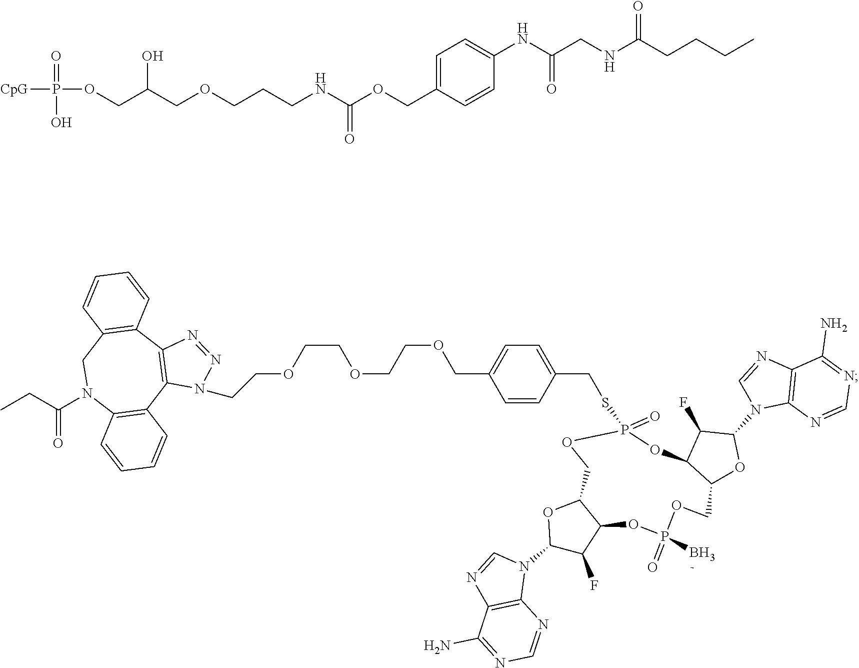 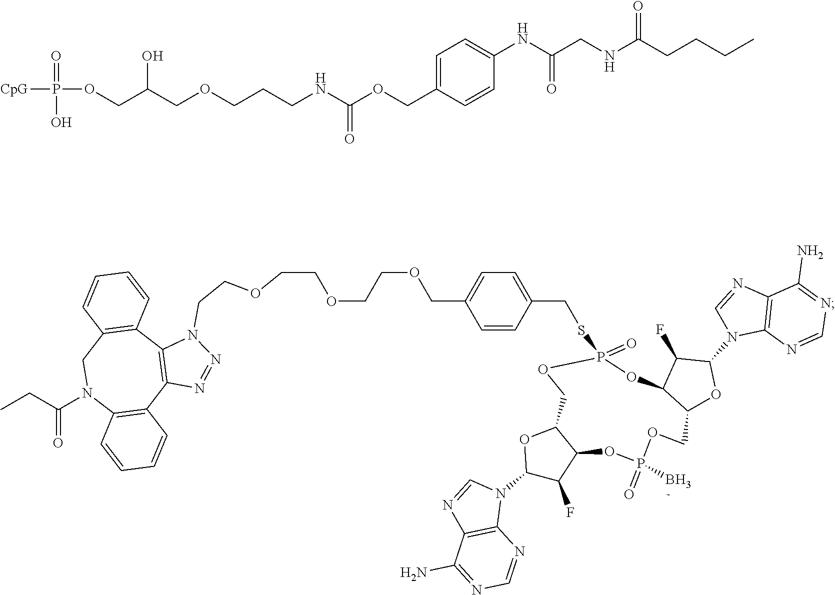 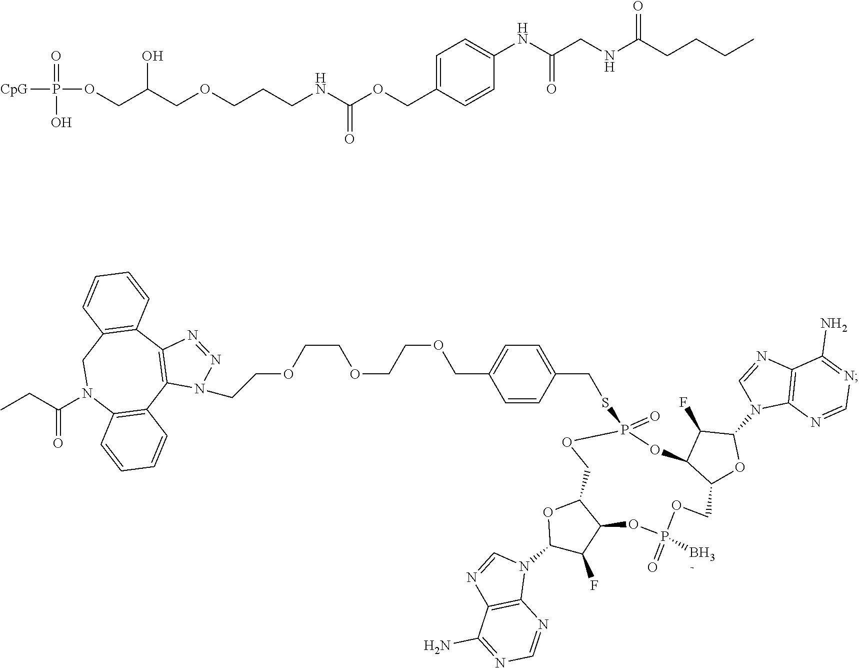 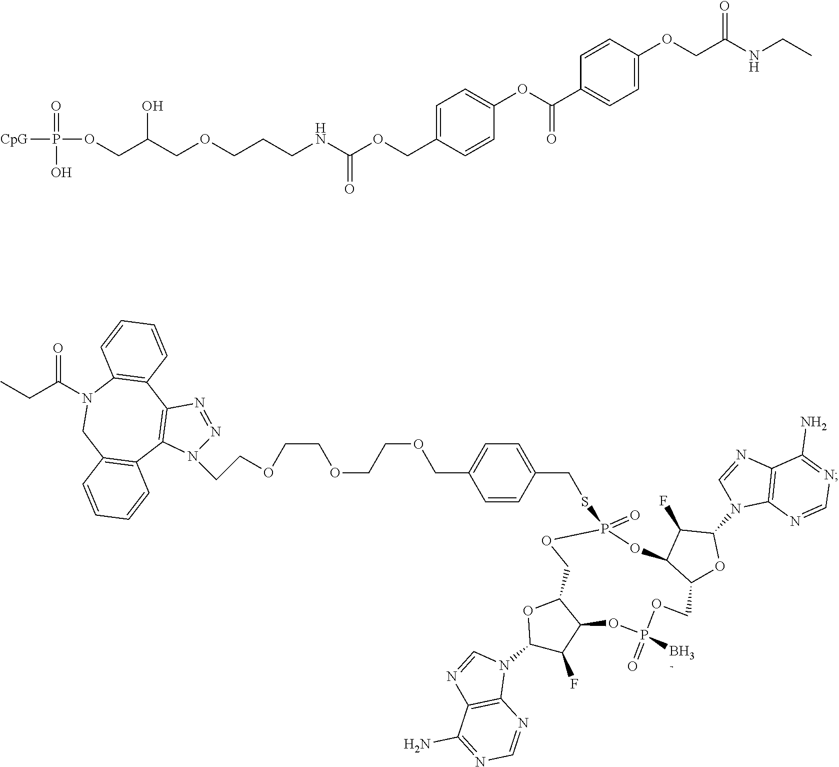 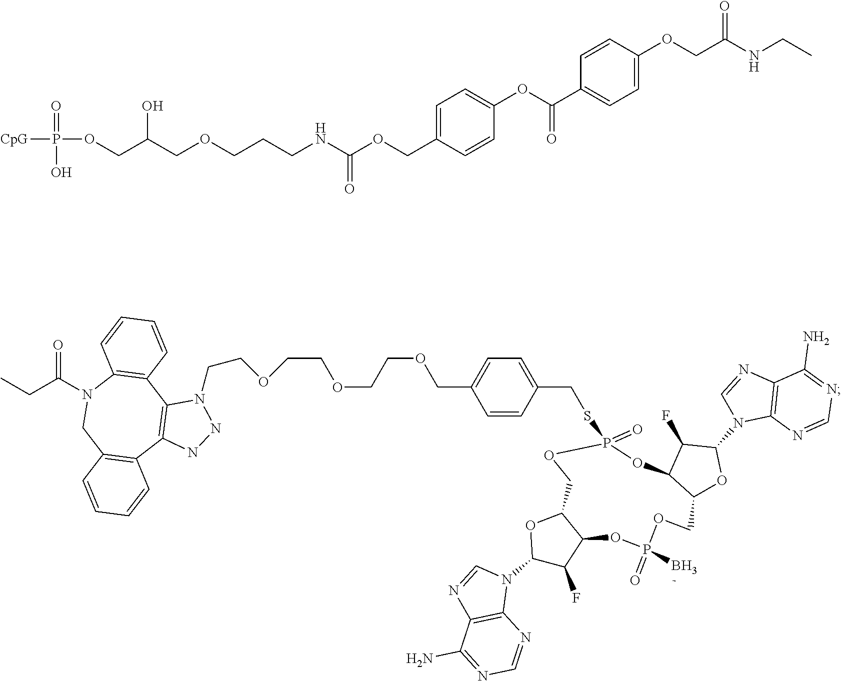 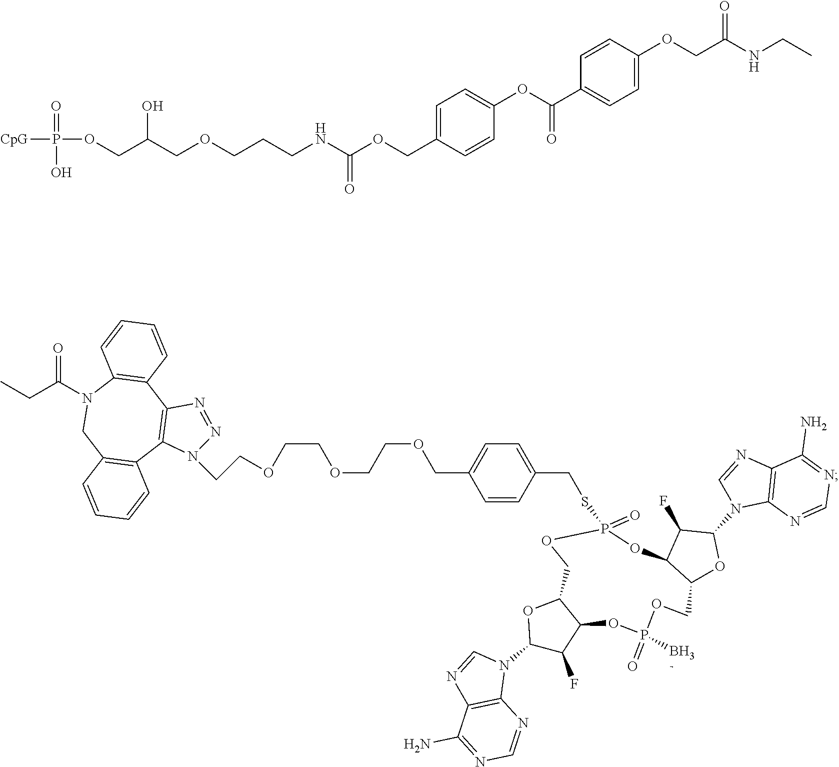 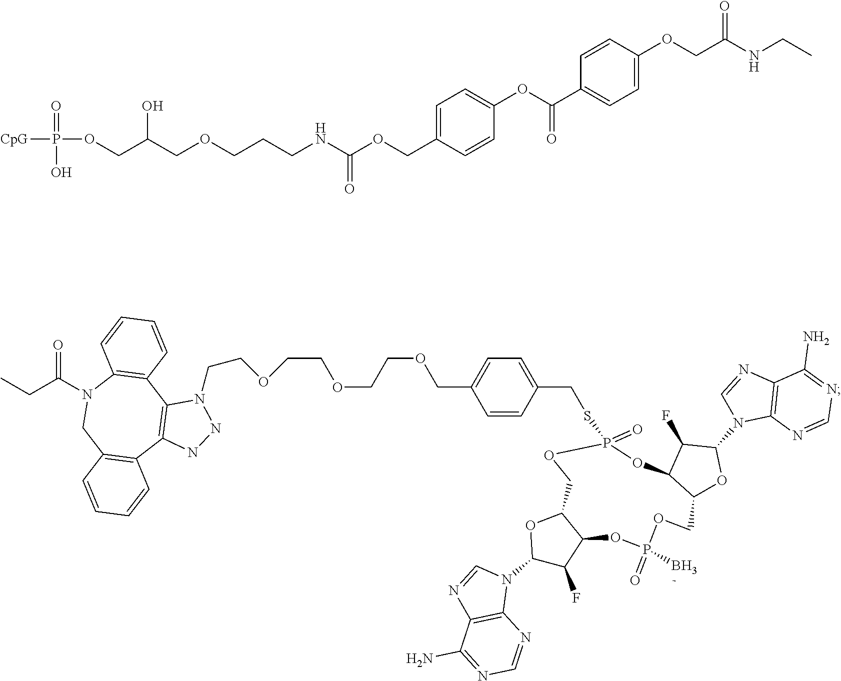 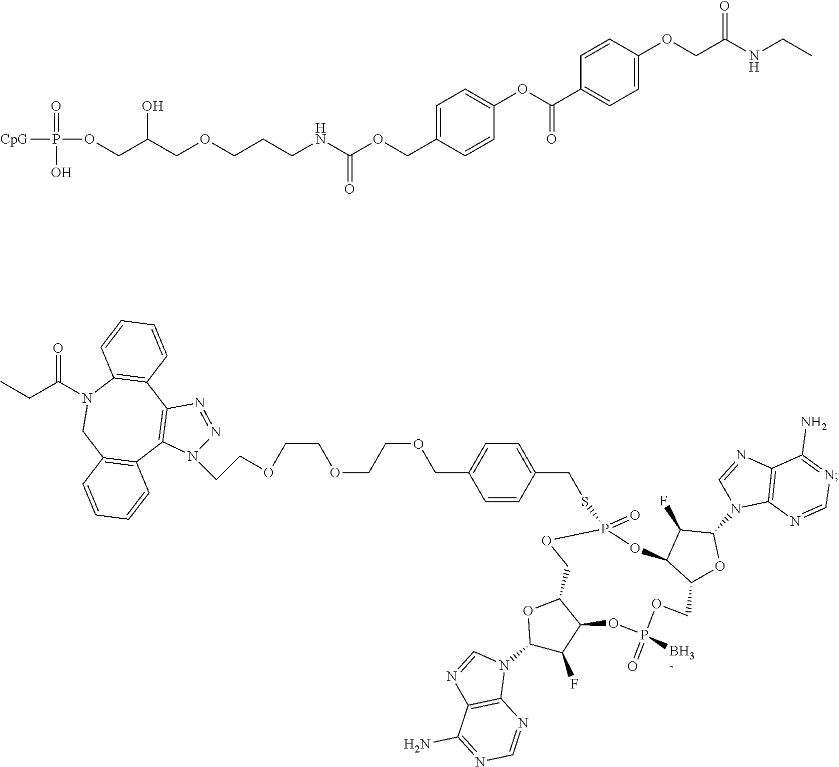 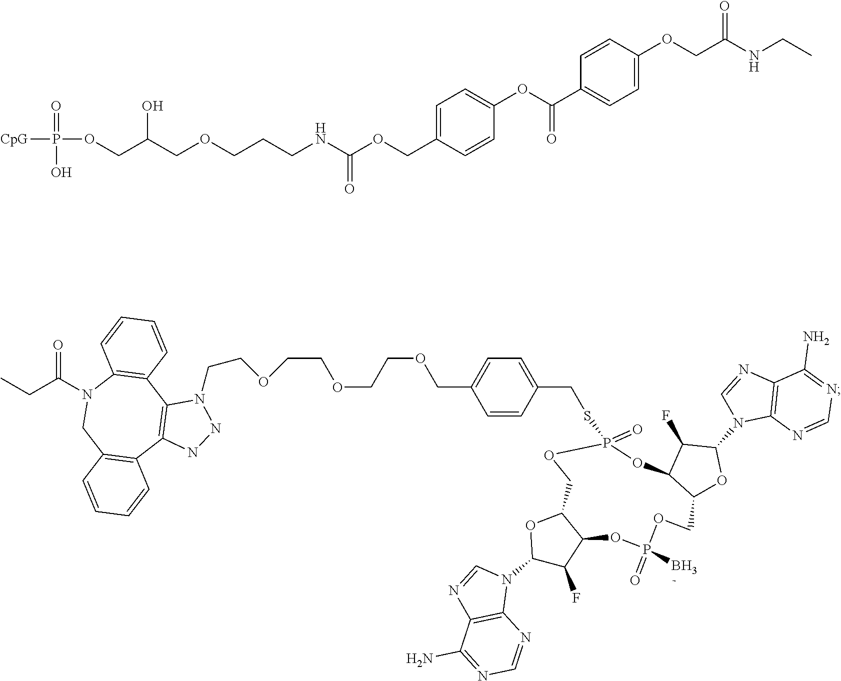 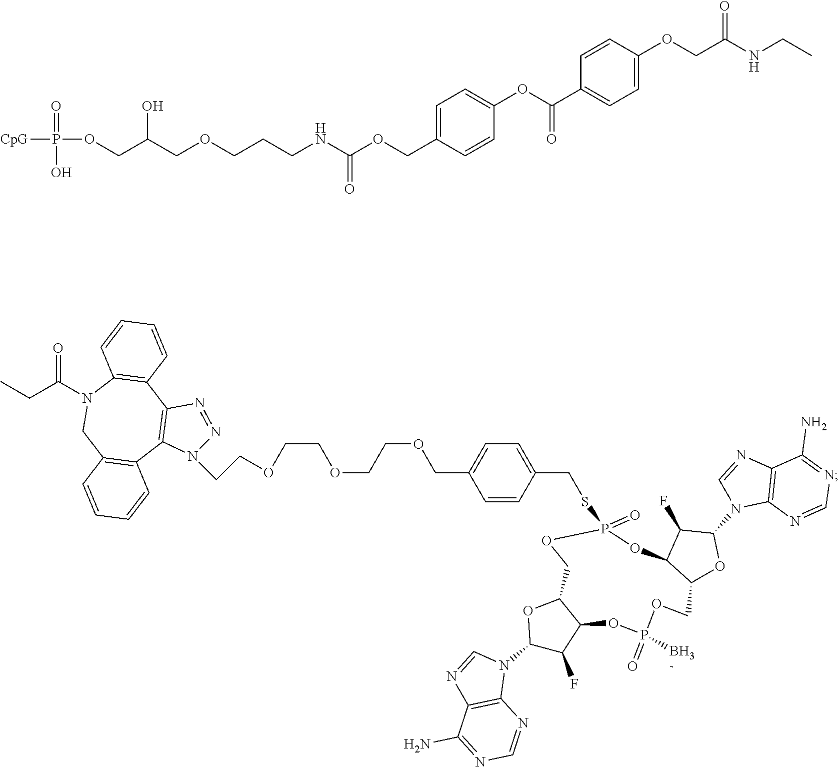 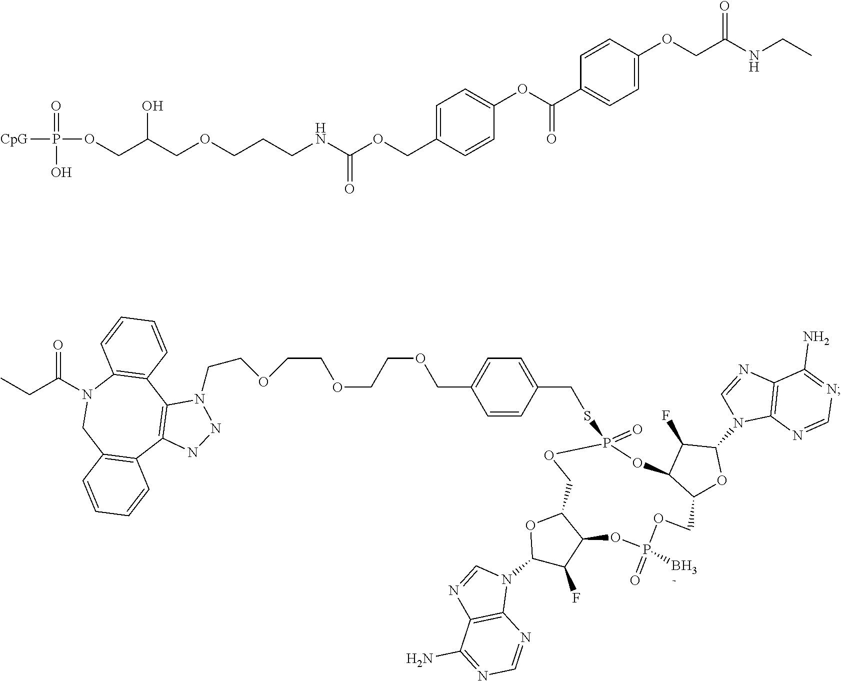 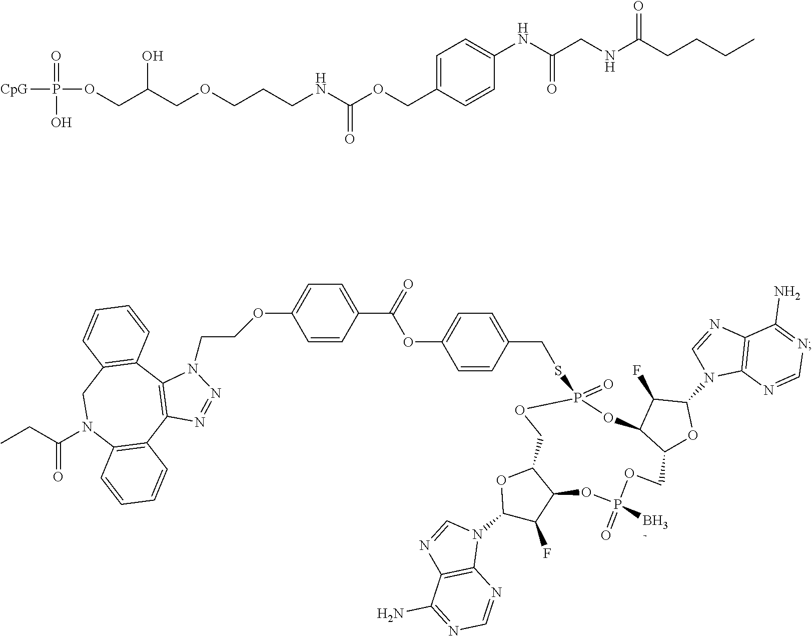 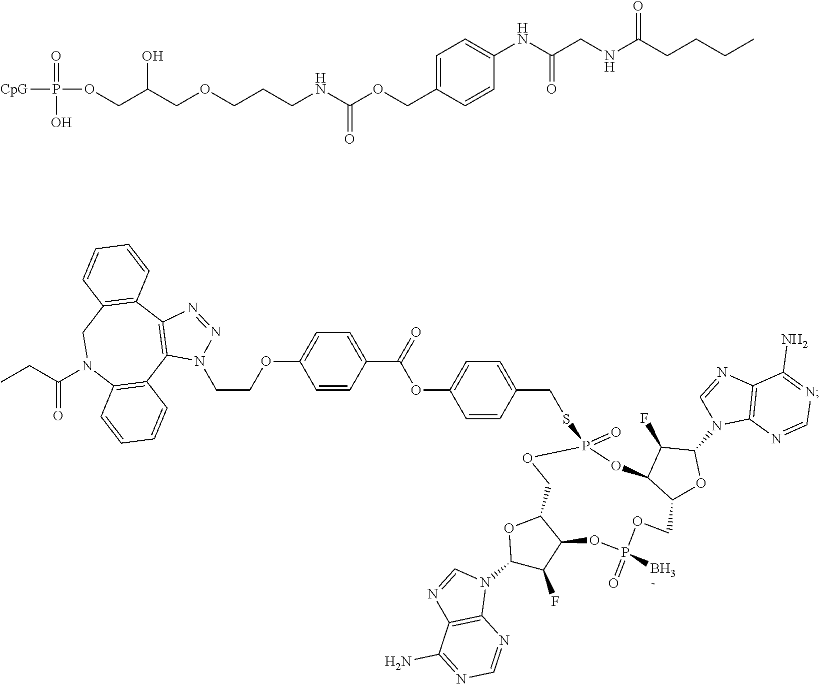 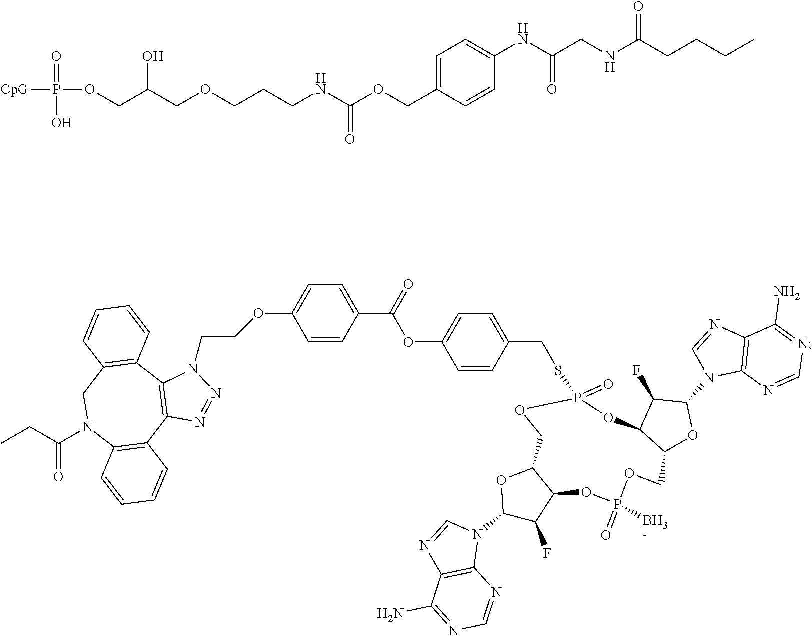 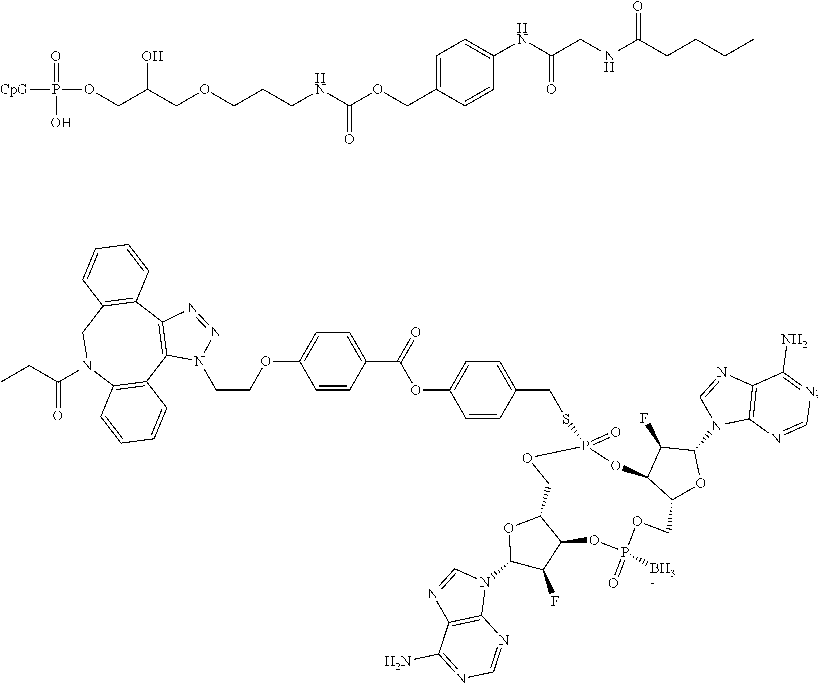 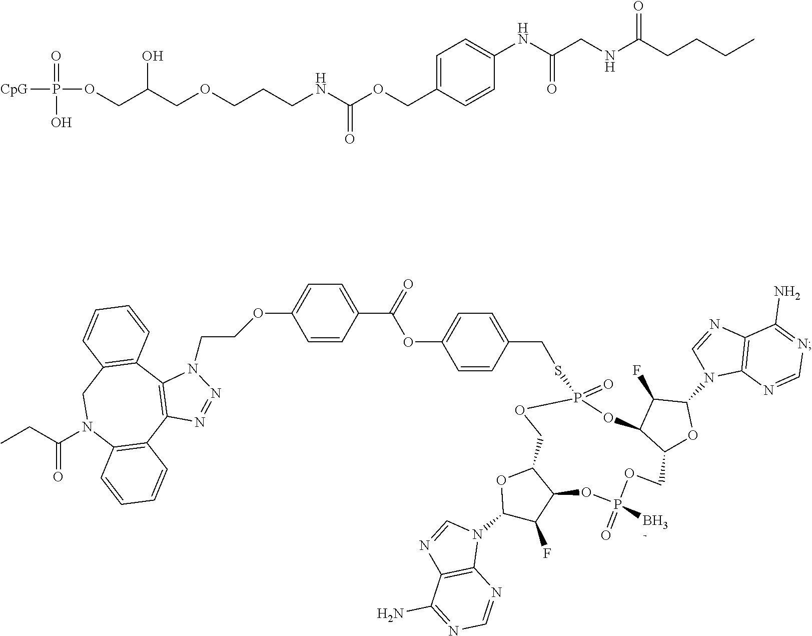 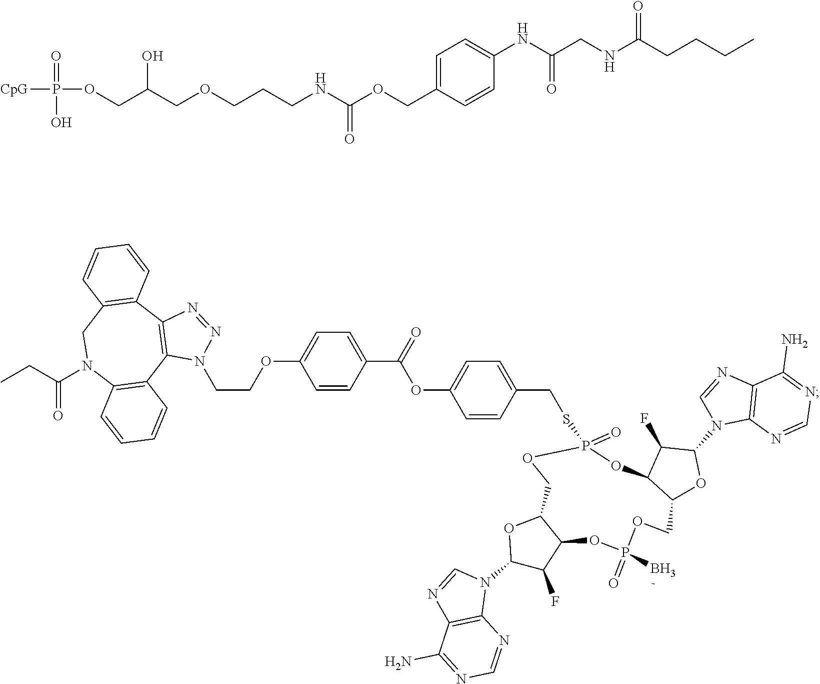 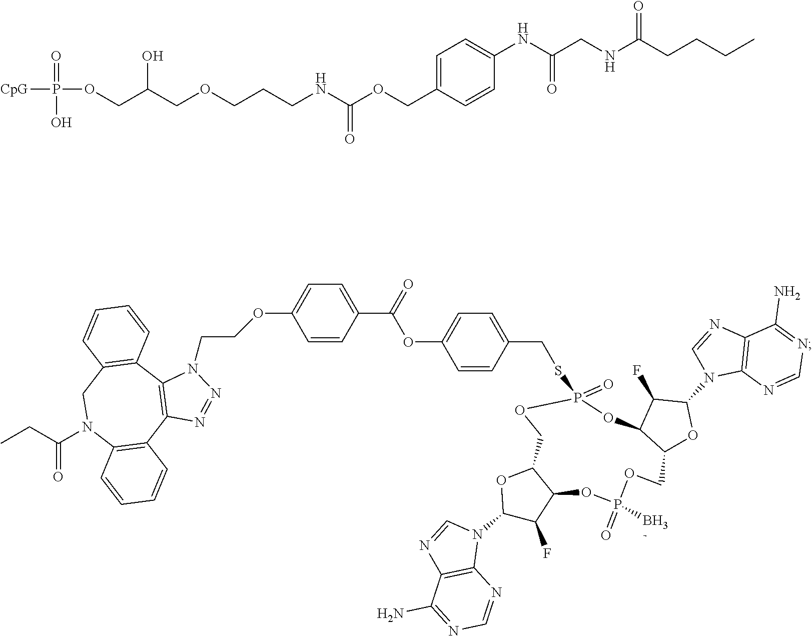 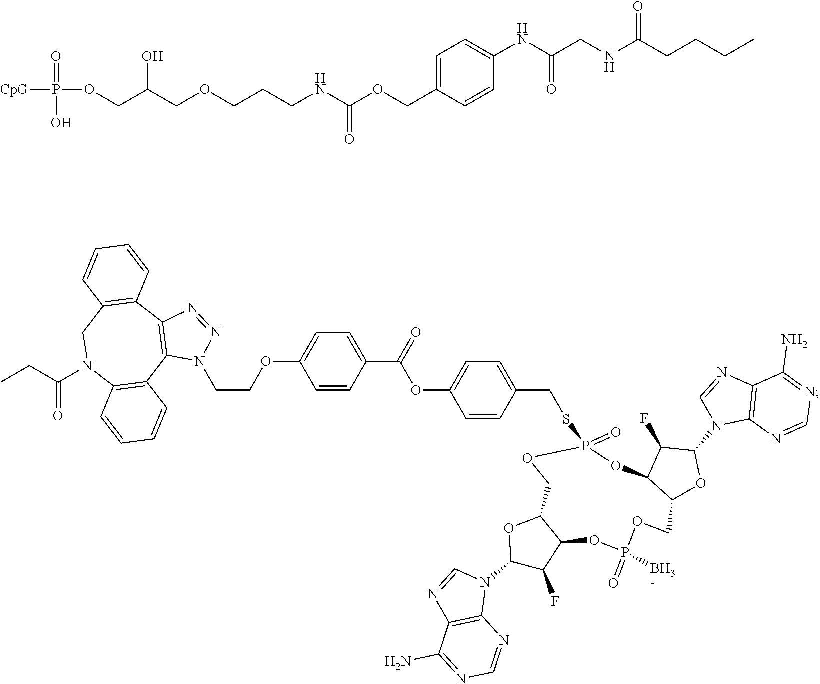 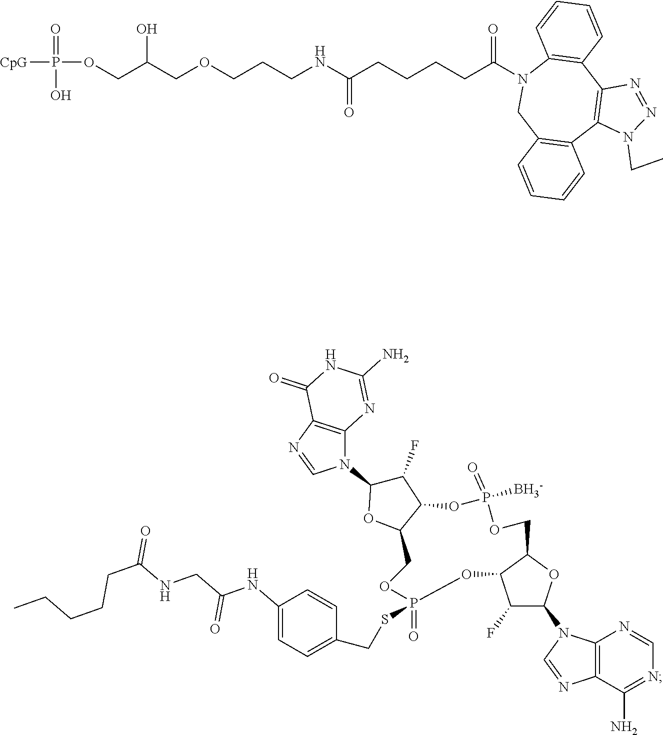 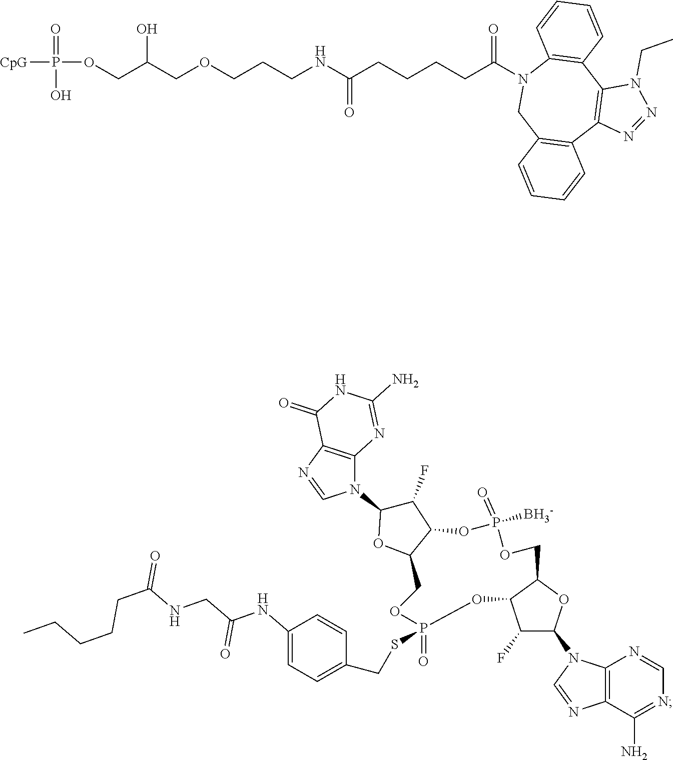 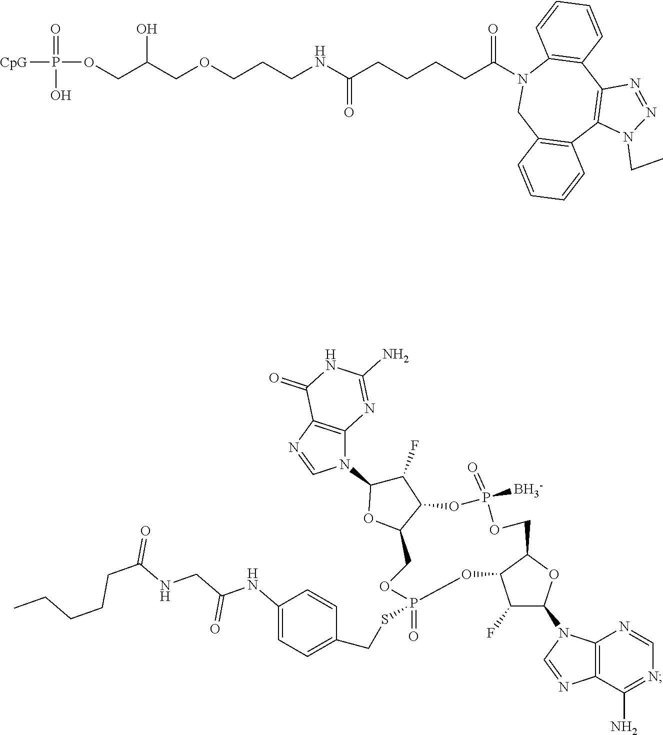 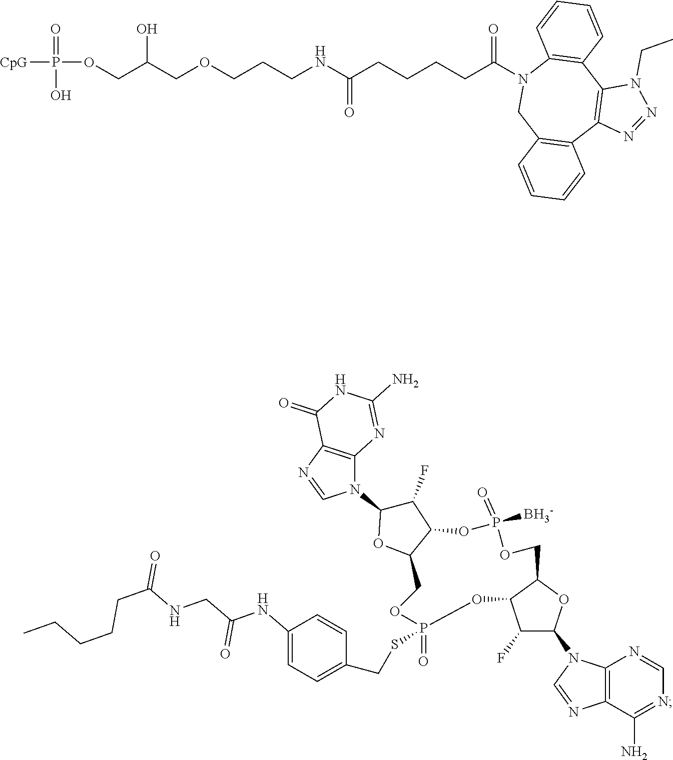 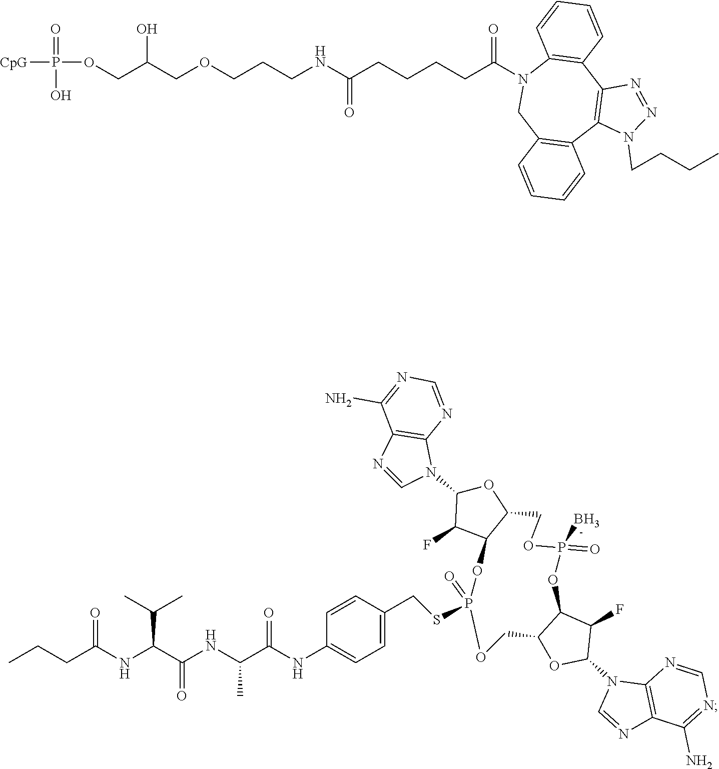 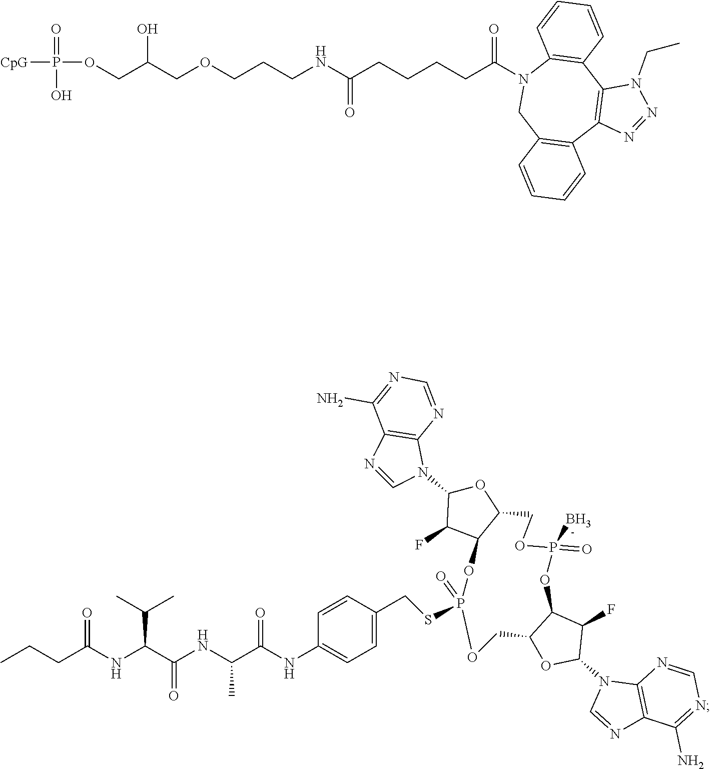 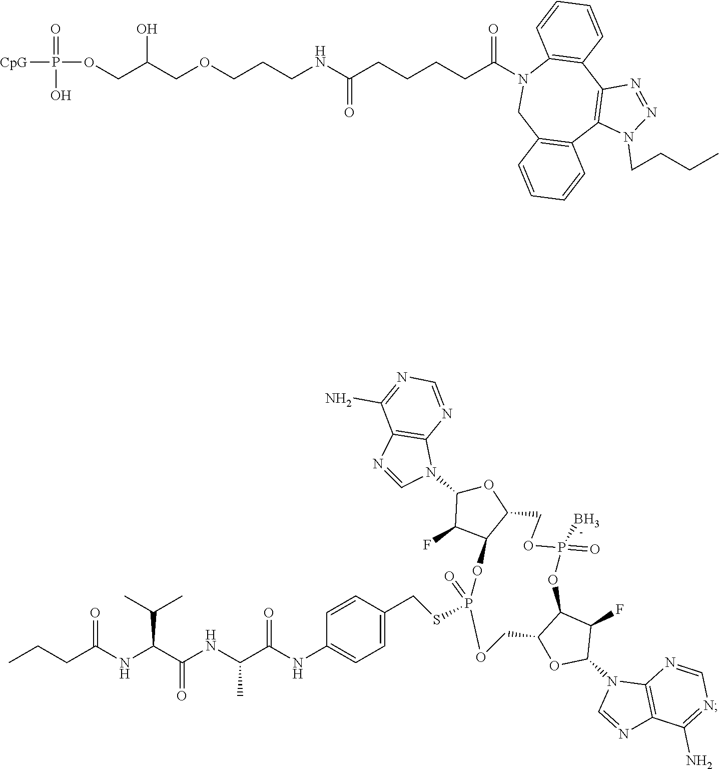 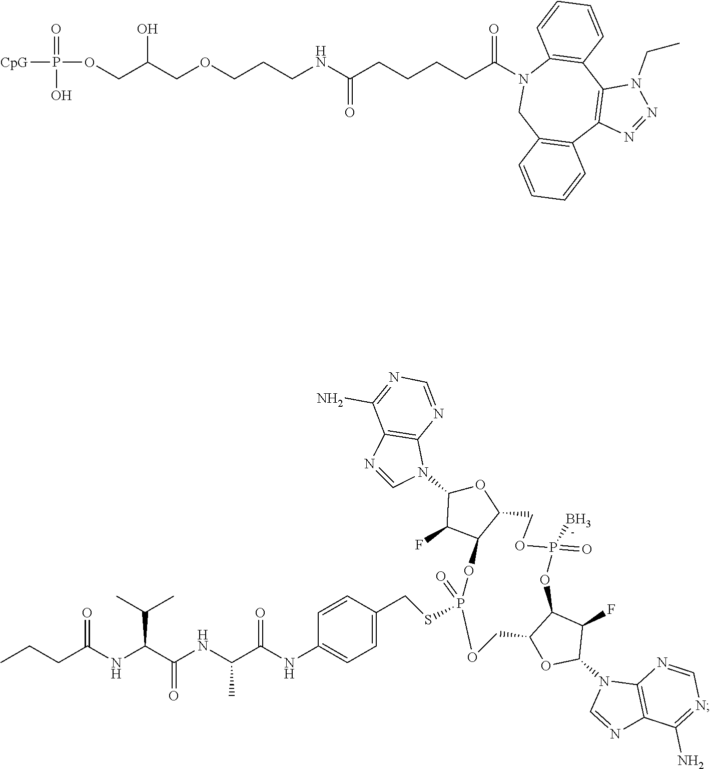 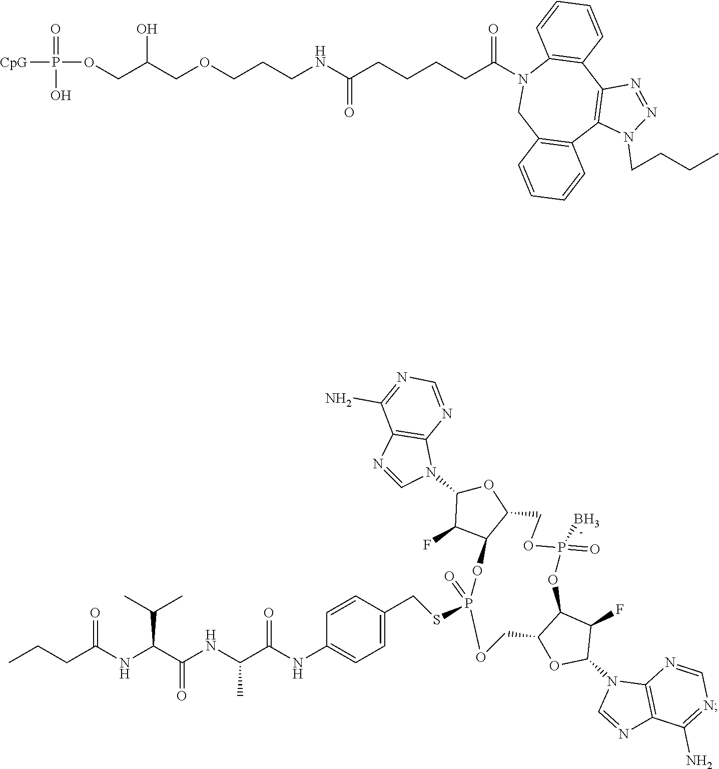 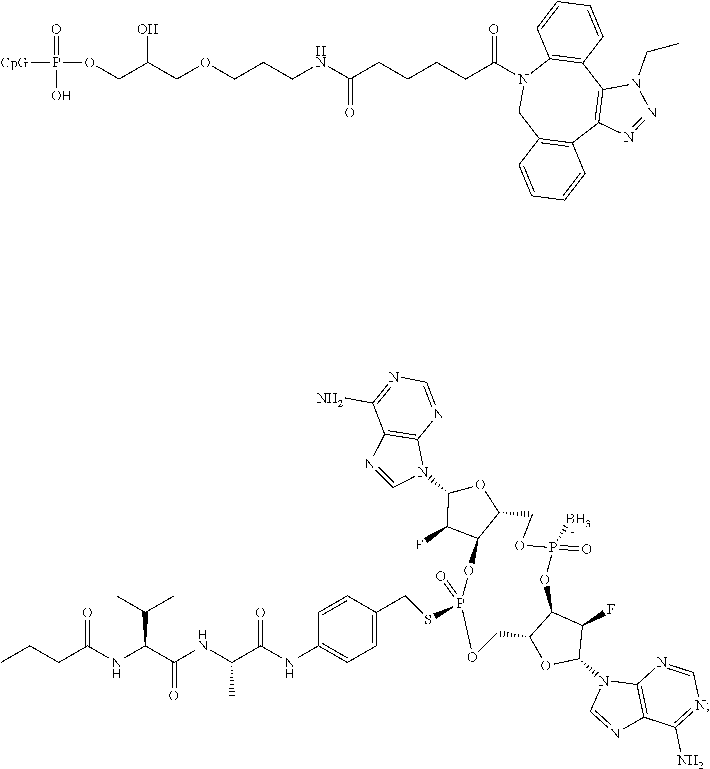 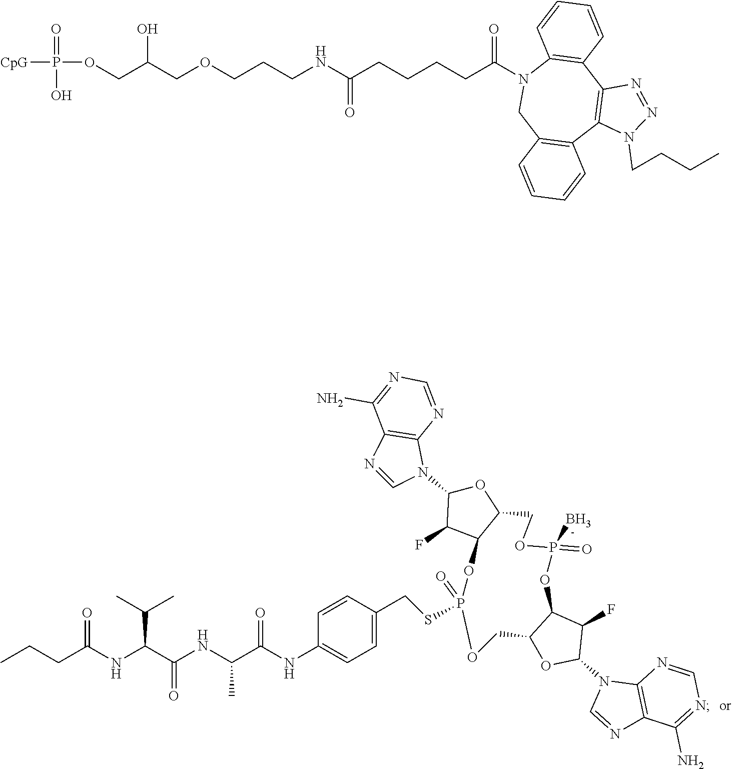 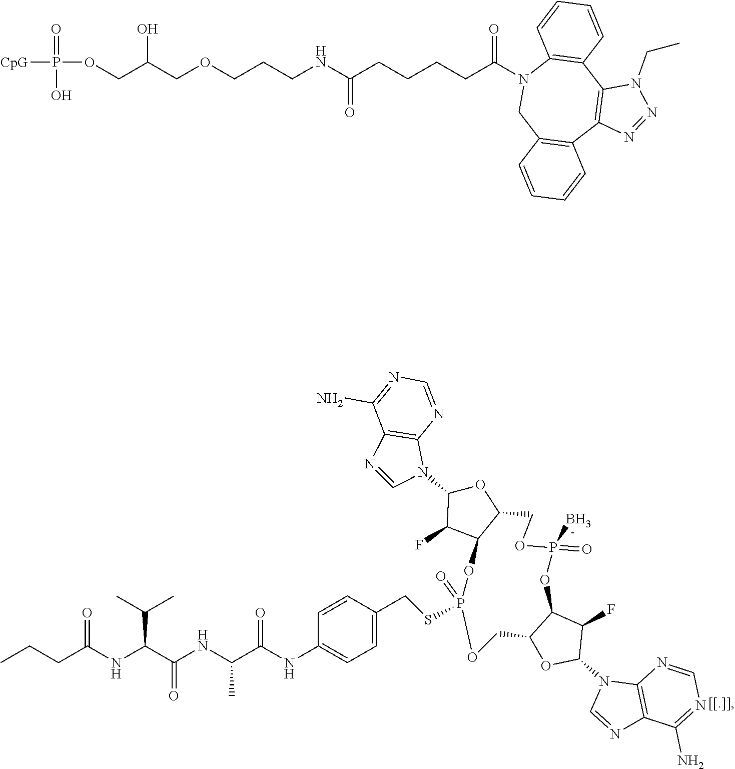
wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide; or 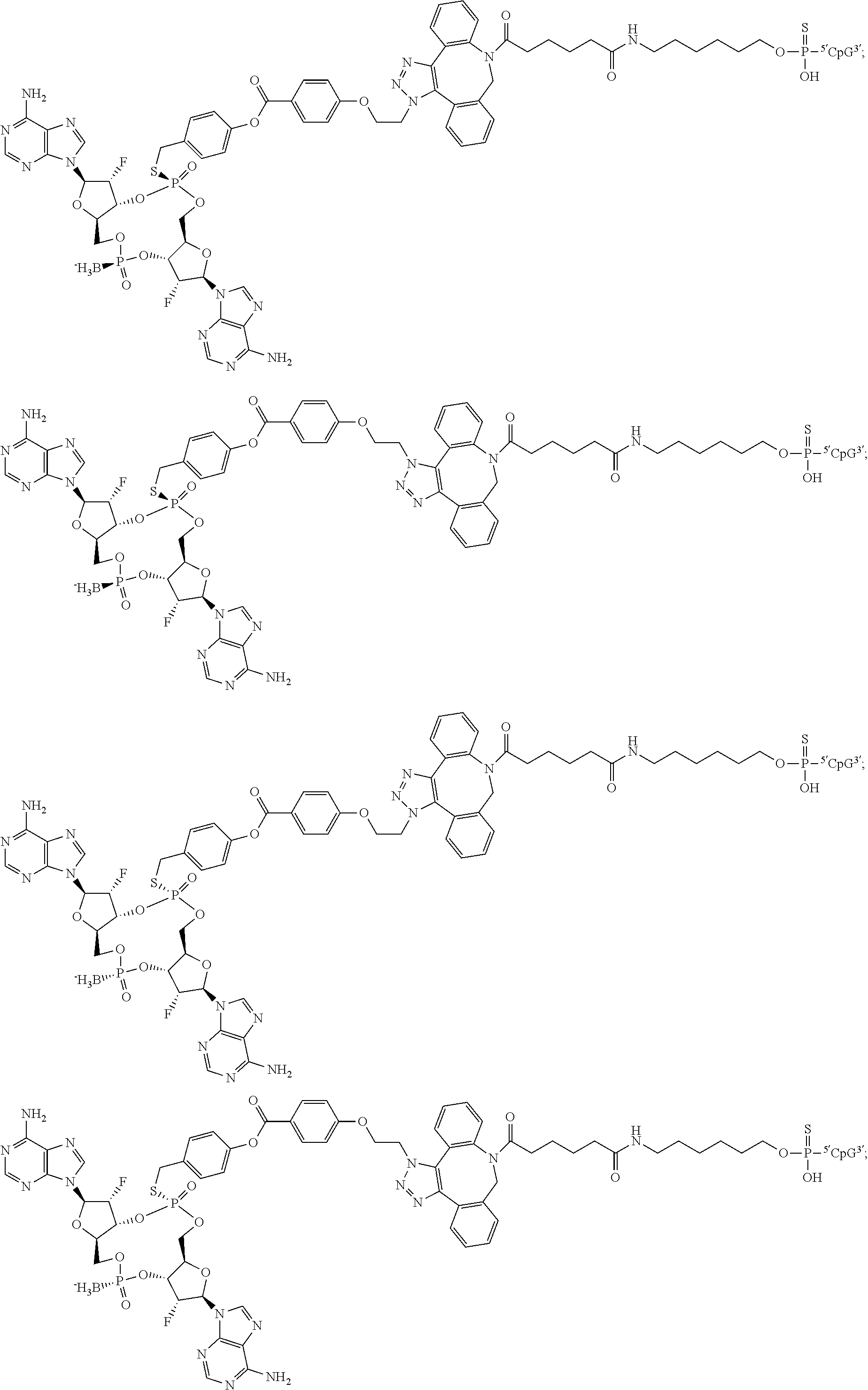 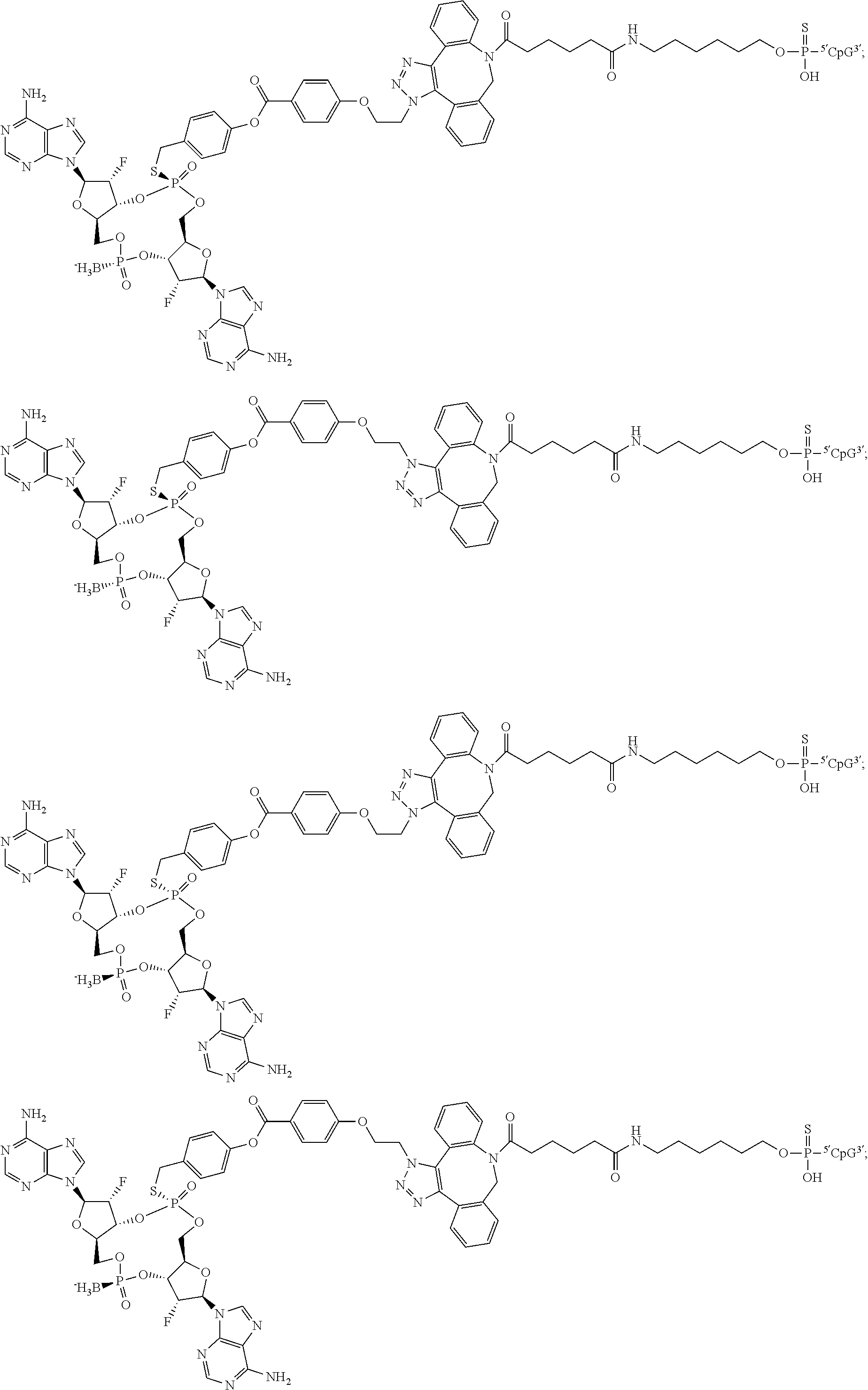 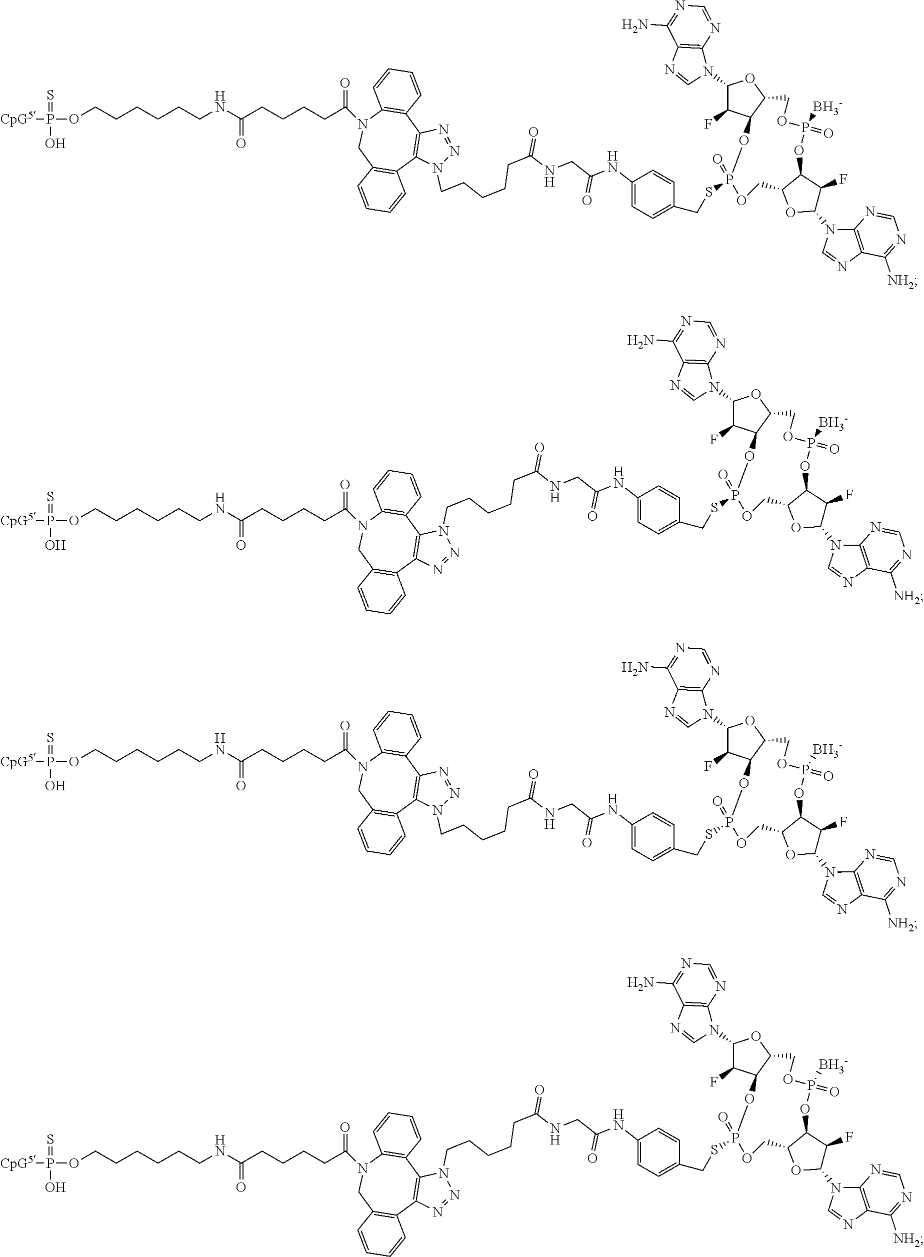 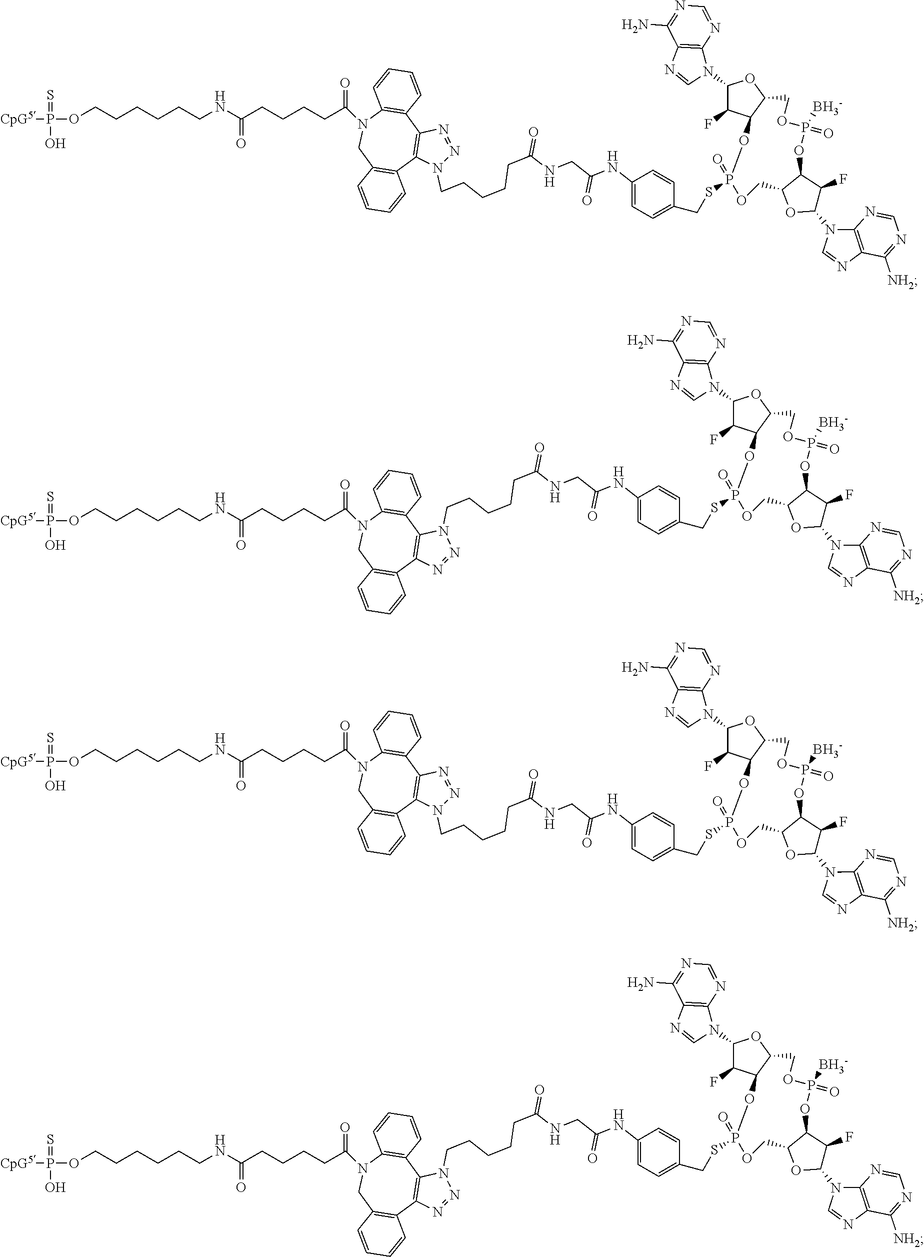 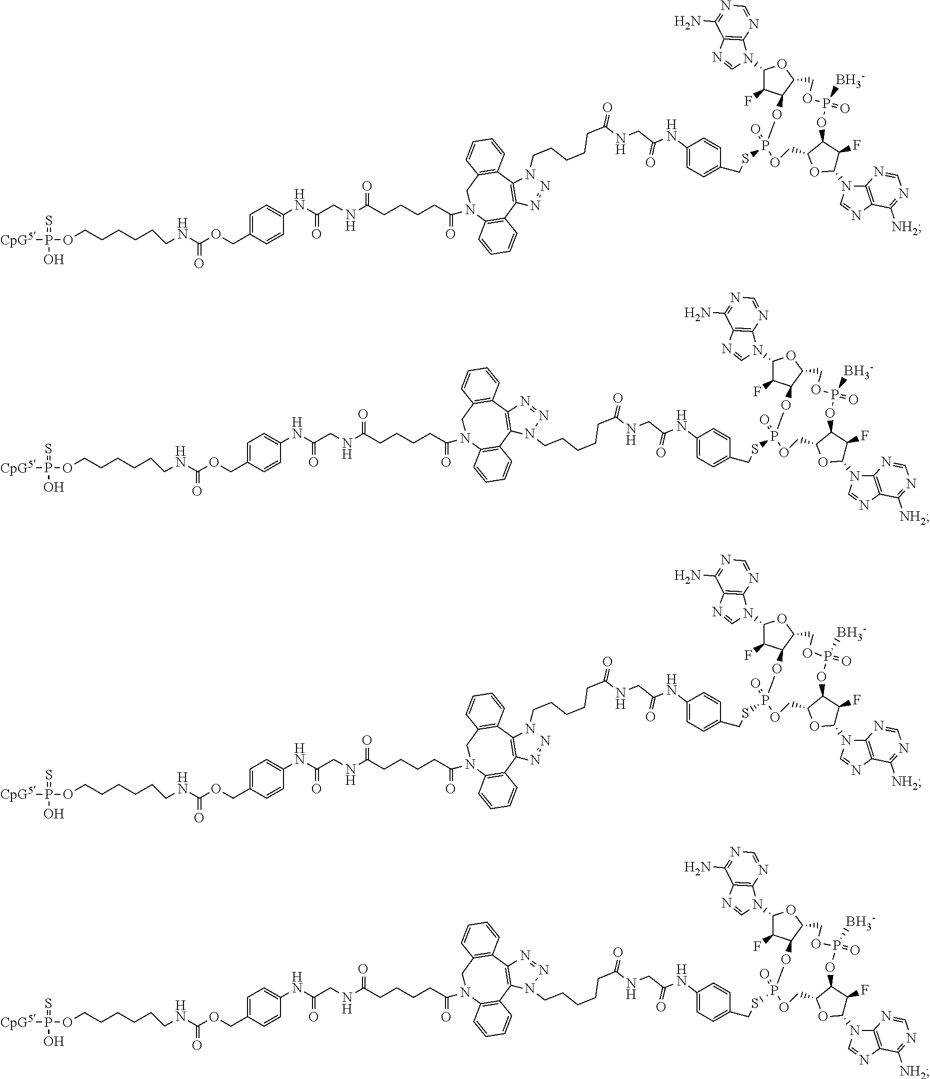 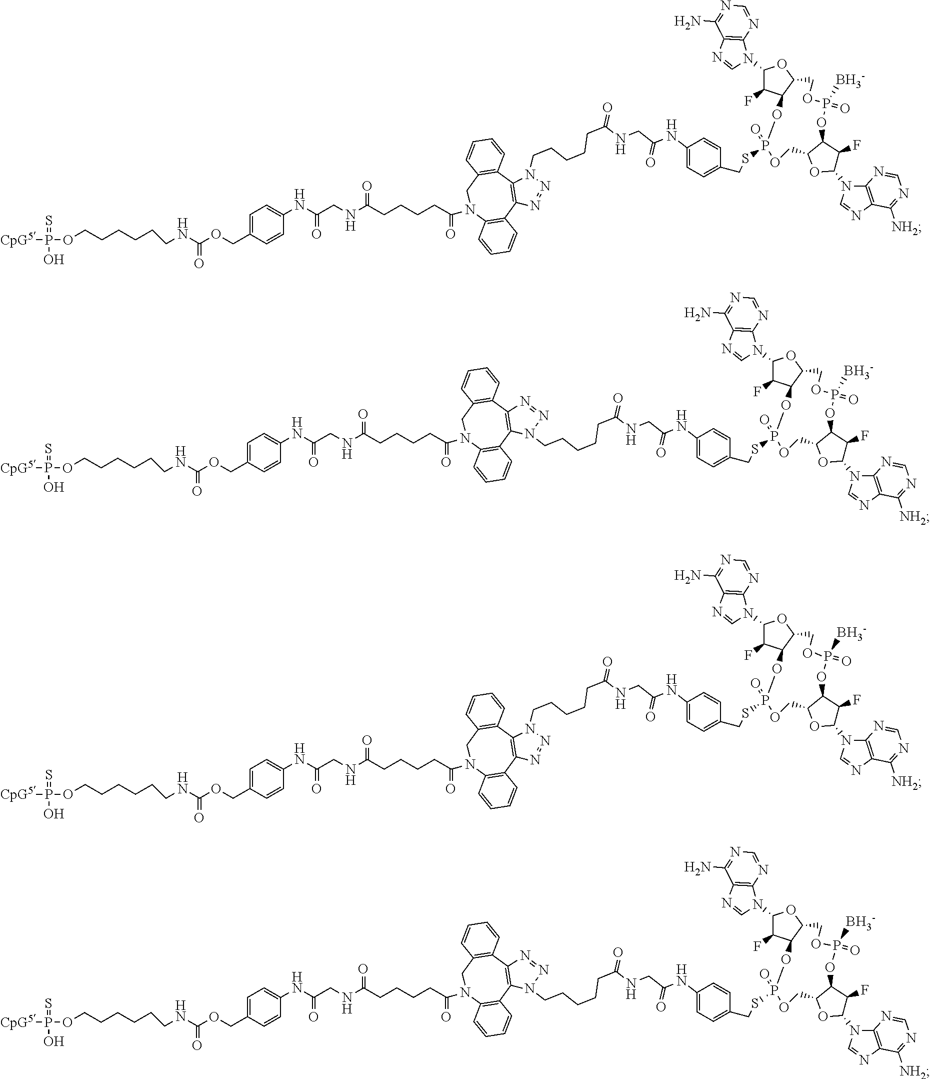 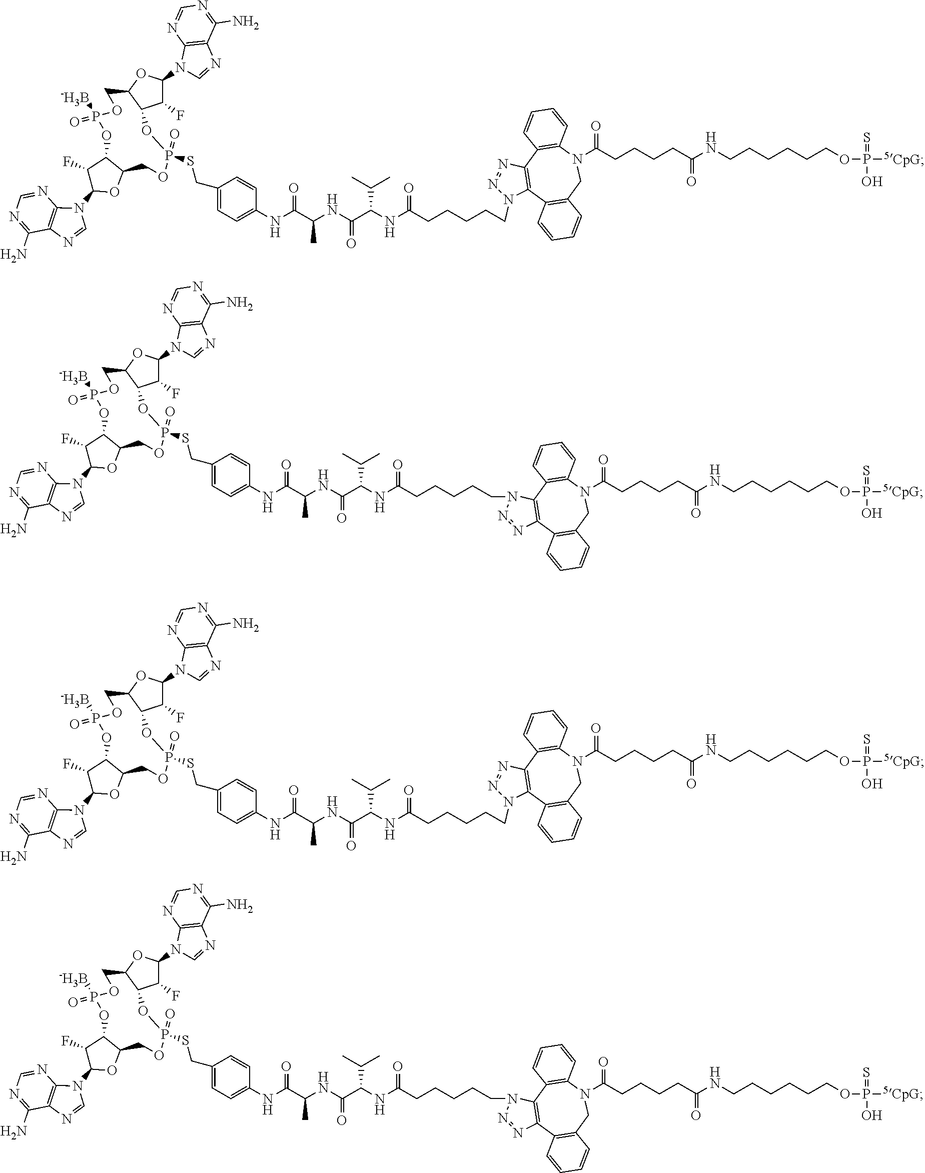 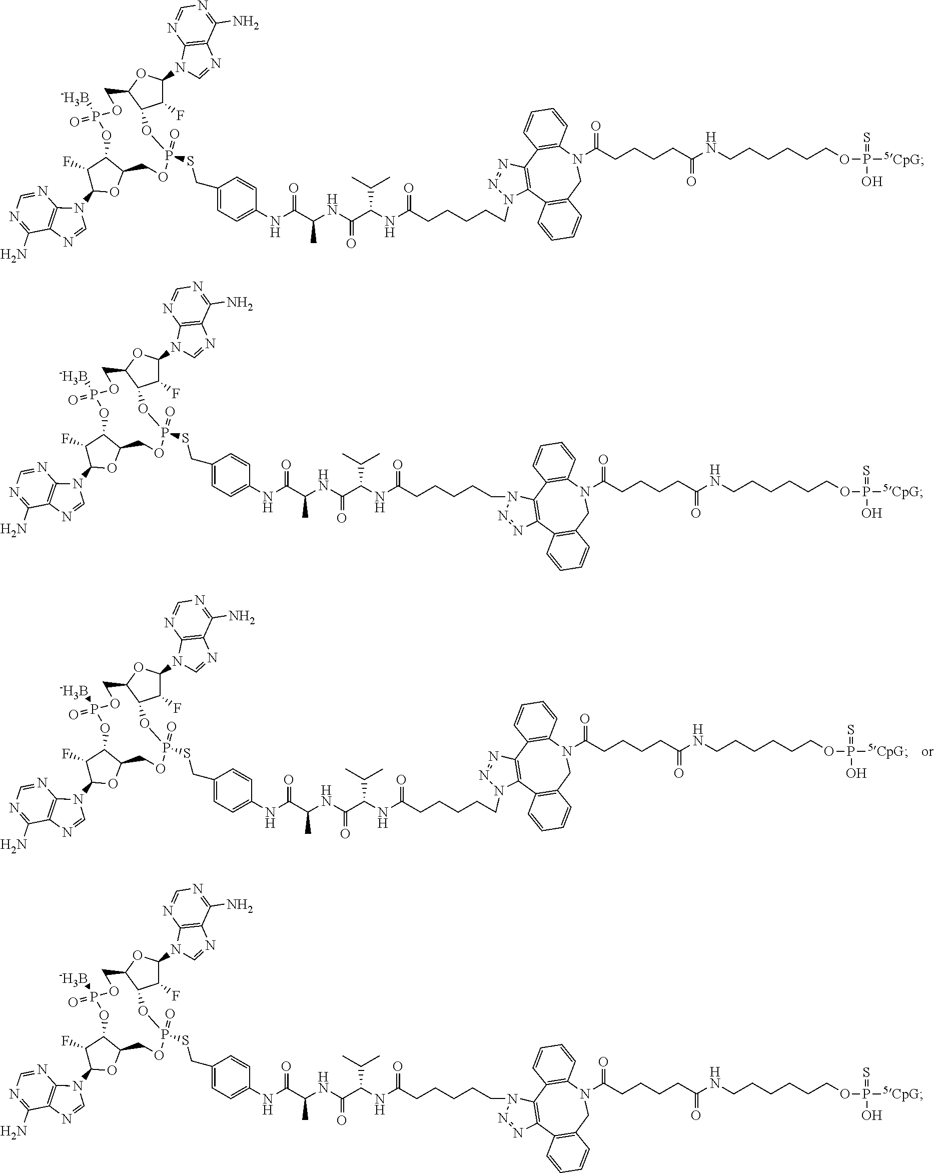
wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at 5′-O of the terminal nucleotide; 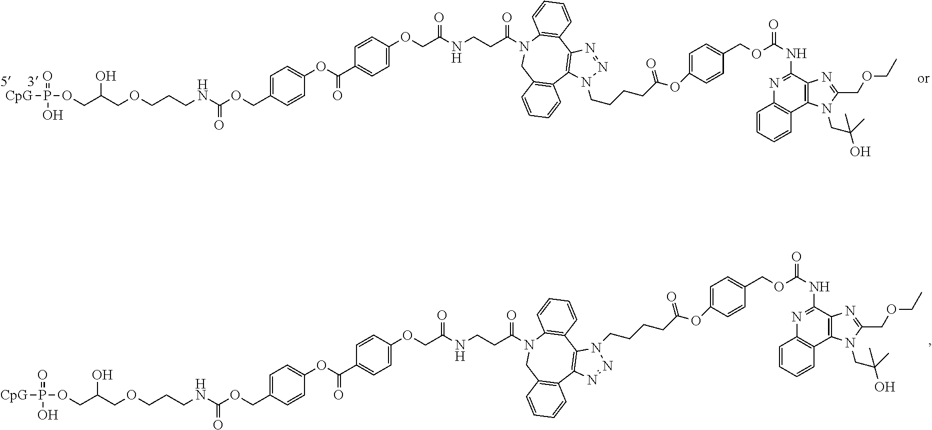
wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide; 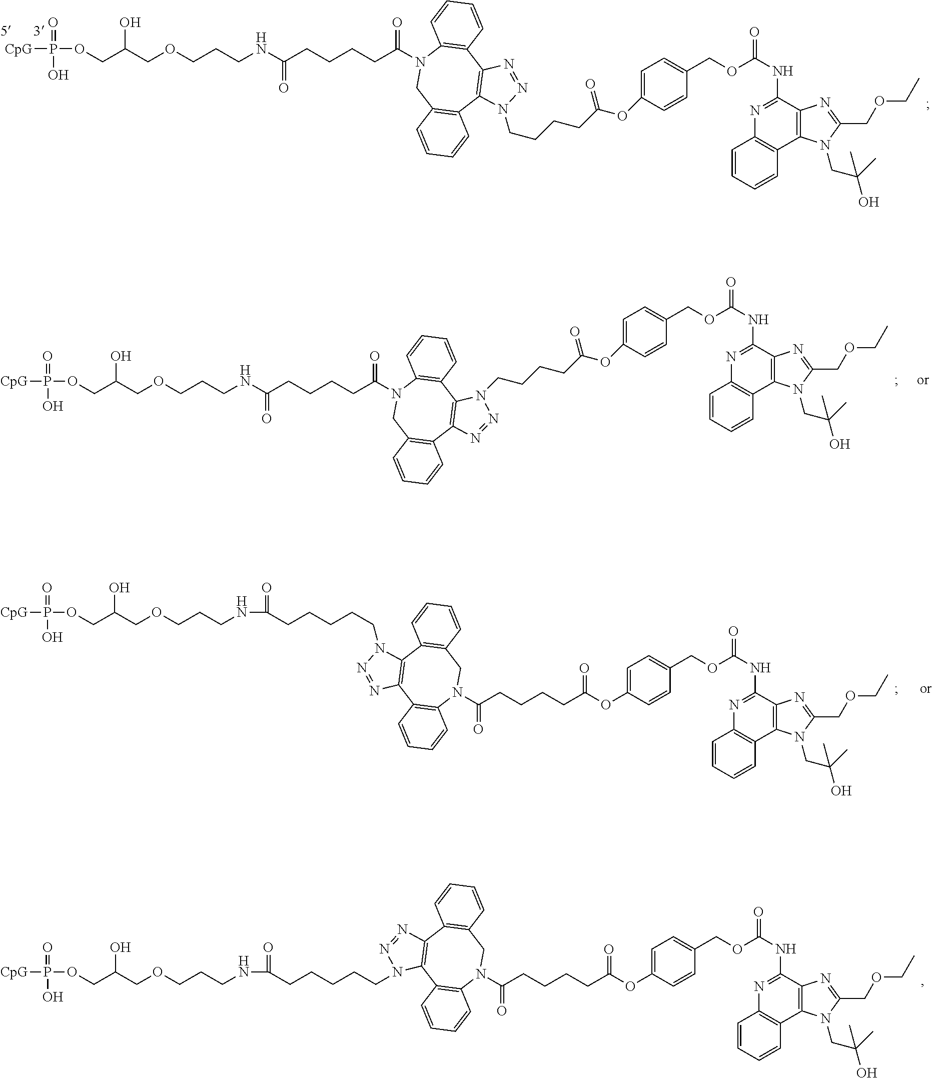
wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide; or

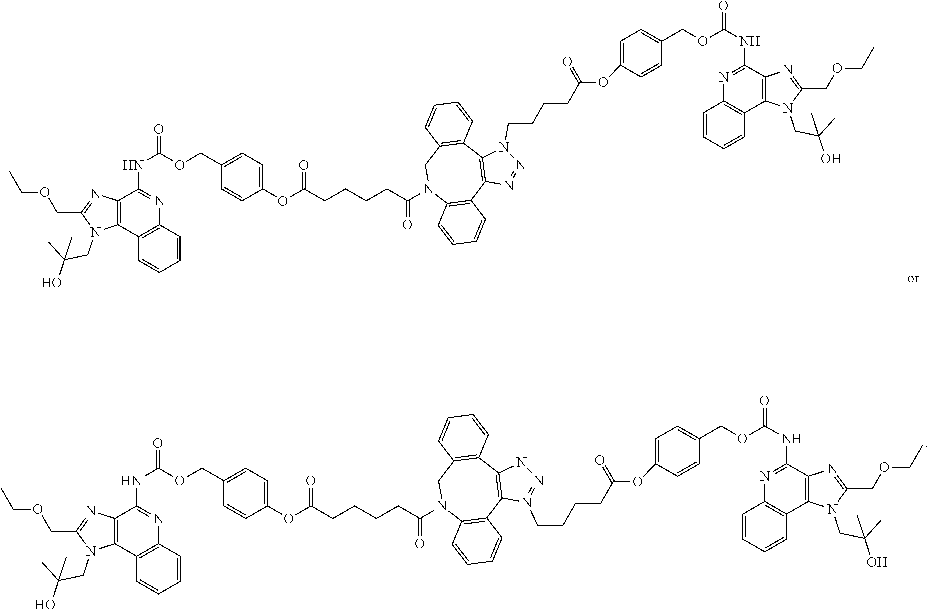

73.-99. (canceled)

100. A STING agonist represented by 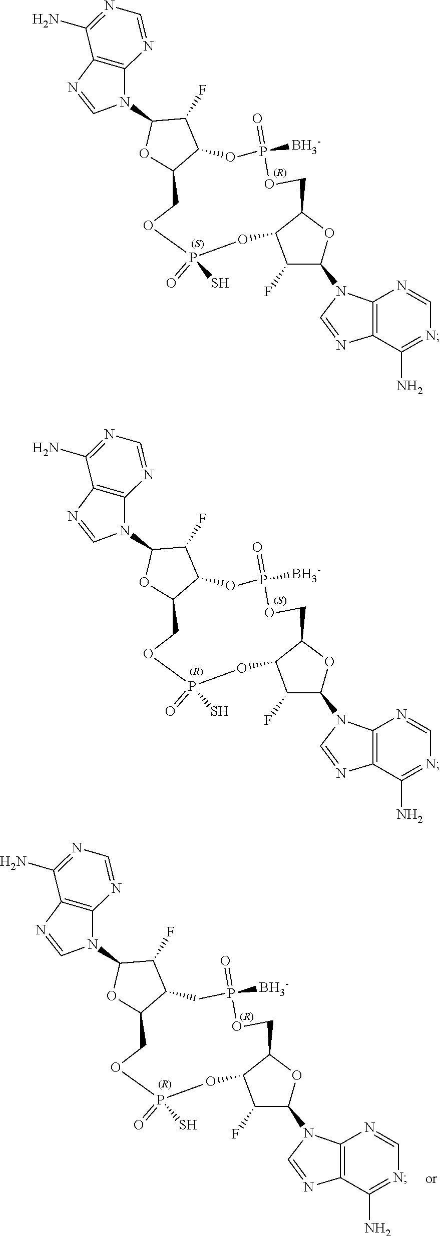 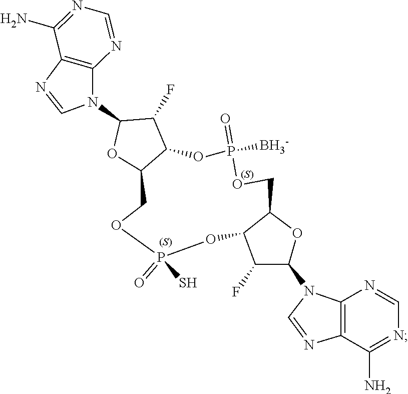
or a tautomer, a mixture of two or more tautomers, or an isotopic variant thereof; or
a pharmaceutically acceptable salt, solvate, hydrate, or prodrug thereof.

101. A compound comprising a linker moiety covalently attached to a therapeutic agent, wherein the compound has a structure of
(i) formula (III):
[Linker]_{b}-A; (III)
or a tautomer, a mixture of two or more tautomers, or an isotopic variant thereof;
wherein
A is a therapeutic agent or an active moiety of a therapeutic agent; or a compound that decomposes to a therapeutic agent;
b is an integer of 1 or 2; wherein when b=2, both groups are directly bound to A;
wherein one or more atoms in each therapeutic agent or compound that decomposes to a therapeutic agent is independently replaced with a covalent bond to a linker, or a chemical group linking the therapeutic agent or compound to a linker;
(ii) formula (XXII): 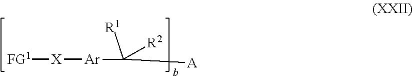
or a tautomer, a mixture of two or more tautomers, or an isotopic variant thereof;
wherein:
Ar is substituted or unsubstituted aryl, or substituted or unsubstituted heteroaryl;
X is a spacer moiety;
each R1 and R2 is, independently, a hydrogen, substituted or unsubstituted alkyl, substituted or unsubstituted alkenyl, substituted or unsubstituted alkynyl, or substituted or unsubstituted aryl; or
R1 and R2, together with the atom to which they are attached, can join together to form a 3-8 membered ring that can optionally contain one or two heteroatoms;
FG1 is a functional group capable of reacting through click chemistry;
b is an integer of 1 or 2; wherein when b=2, both groups are directly bound to A; and
A is a STING agonist
wherein one or more atoms in STING agonist is independently replaced with a covalent bond to a linker; or
(iii) formula (V): 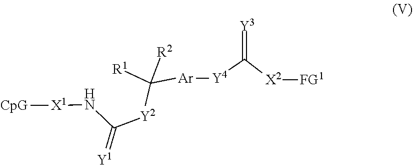
or a tautomer, a mixture of two or more tautomers, or an isotopic variant thereof;
wherein:
Ar is substituted or unsubstituted aryl, or substituted or unsubstituted heteroaryl;
X1 and X2 are each independently a spacer moiety;
each R1 and R2 is independently, a hydrogen, substituted or unsubstituted alkyl, substituted or unsubstituted alkenyl, substituted or unsubstituted alkynyl, or substituted or unsubstituted aryl; or
R1 and R2, together with the atom to which they are attached, can join together to form a 3-8 membered ring that can optionally contain one or two heteroatoms;
Y1 is O, NH or S;
Y2 is O, NH or S;
Y3 is O, NH or S;
Y4 is O, NH or S;
FG1 is a functional group capable of reacting through click chemistry; and
CpG is a TLR9 agonist oligodeoxynucleotide wherein the CpG is covalently bound to X1 through a 3′-O or 5′-O of a terminal nucleotide; or
(iv) formula (VI):
CpG-X—FG¹ (VI)
or a tautomer, a mixture of two or more tautomers, or an isotopic variant thereof;
wherein:
X is a spacer moiety;
FG¹ is a functional group capable of reacting through click chemistry; and
CpG is a TLR9 agonist oligodeoxynucleotide wherein the CpG is covalently bound to X through a 3′-O or 5′-O of a terminal nucleotide of the CpG, or
(v) a TLR9 agonist derivative that is released from the conjugates of claim 1, wherein the released TLR9 agonist has a structure of formula (XX): 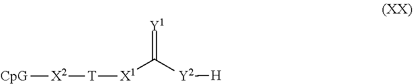
or a tautomer, a mixture of two or more tautomers, a regioisomer, a mixture of two or more regioisomers, or an isotopic variant thereof; or a pharmaceutically acceptable salt, solvate, hydrate, or prodrug thereof;
wherein:
X¹ and X² are each independently a spacer moiety;
Y¹ and Y² are each independently O or S;
T is a triazole functional group; and
CpG is a TLR9 agonist oligodeoxynucleotide;
wherein one or more atoms in the CpG is independently replaced with a covalent bond to X²; or
(vi) a TLR9 agonist having a structure of formula (I): 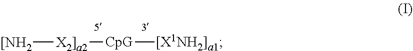
or a tautomer, a mixture of two or more tautomers, or an isotopic variant thereof; or a pharmaceutically acceptable salt, solvate, hydrate, or prodrug thereof;
wherein:
X¹, X² are each independently a spacer moiety;
a1 is an integer of 0 or 1;
a2 is an integer of 0 or 1;
wherein a1+a2 is 1 or 2; and
CpG is a TLR9 agonist oligodeoxynucleotide;
wherein one or more atoms in the CpG is independently replaced with a covalent bond to X¹ and/or X².

102.-120. (canceled)

121. The compound of claim 101, wherein the compound is: 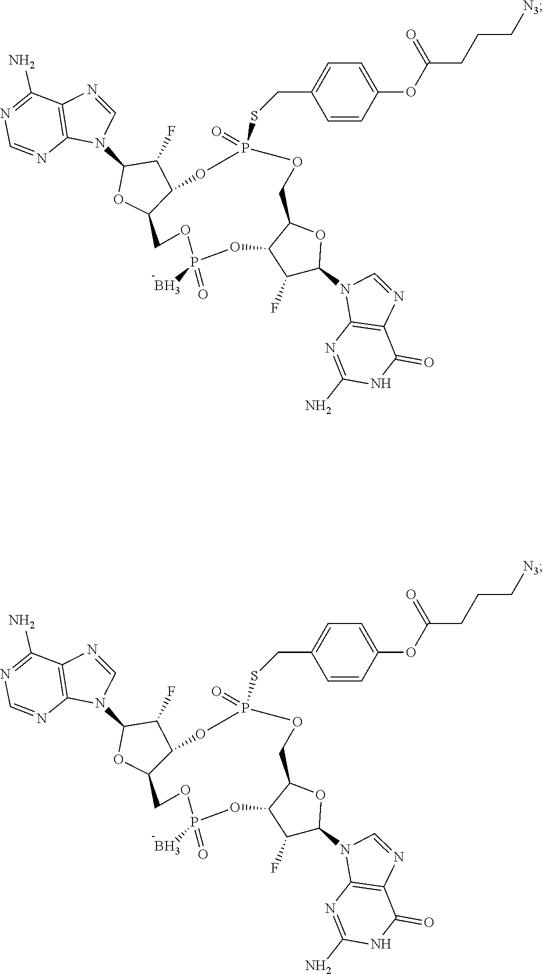 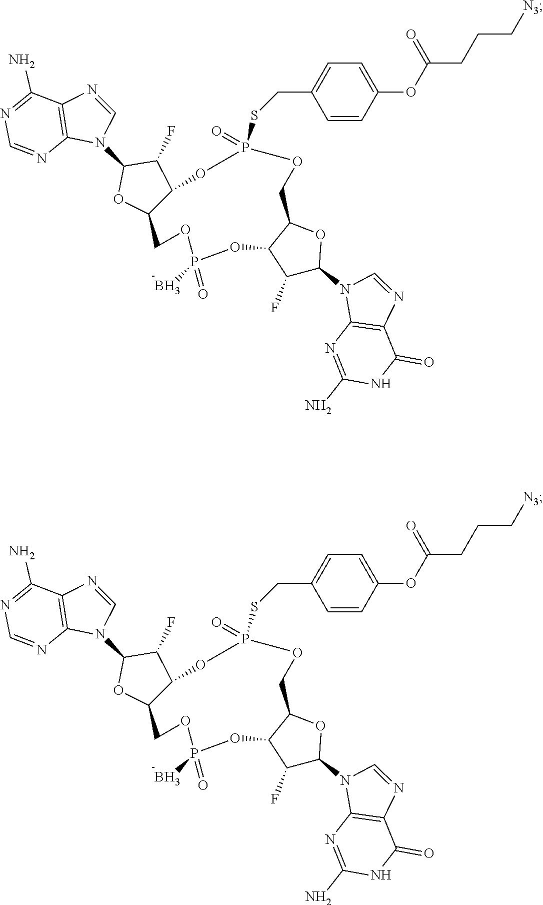 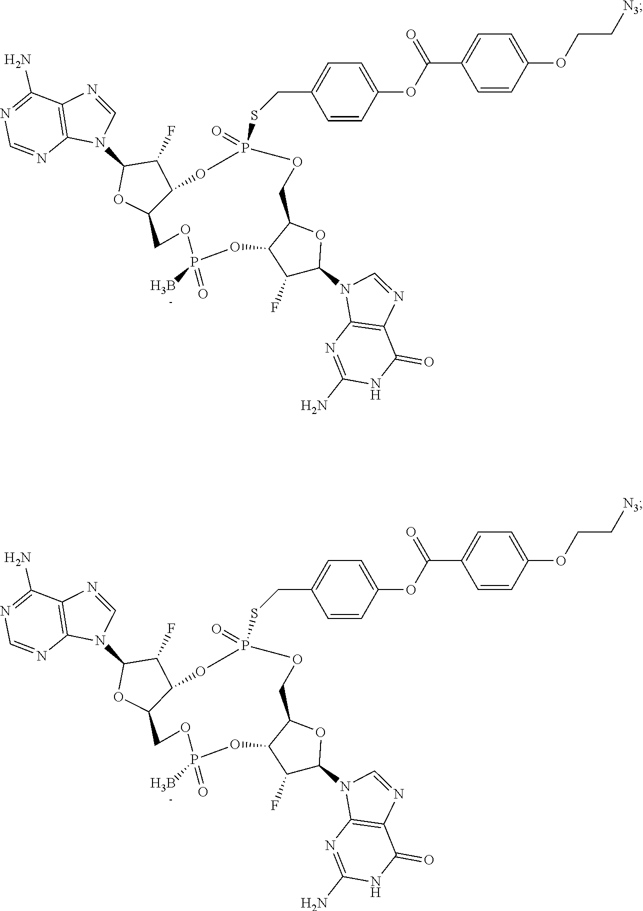 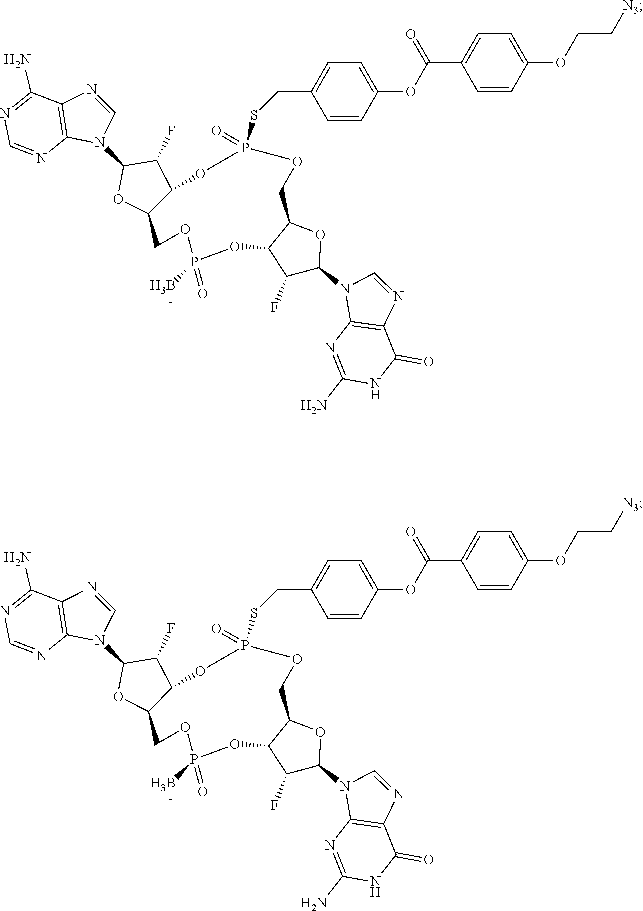 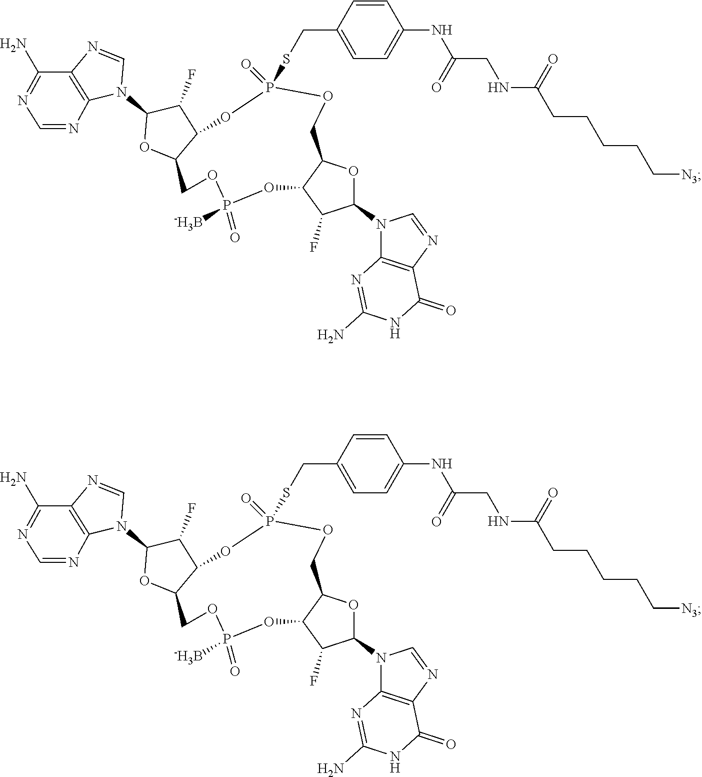 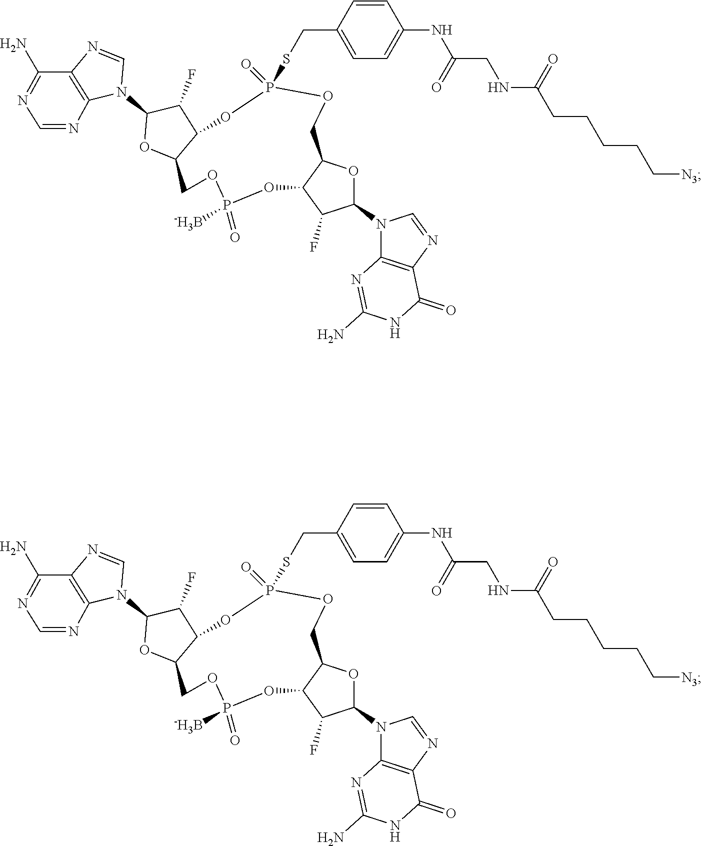 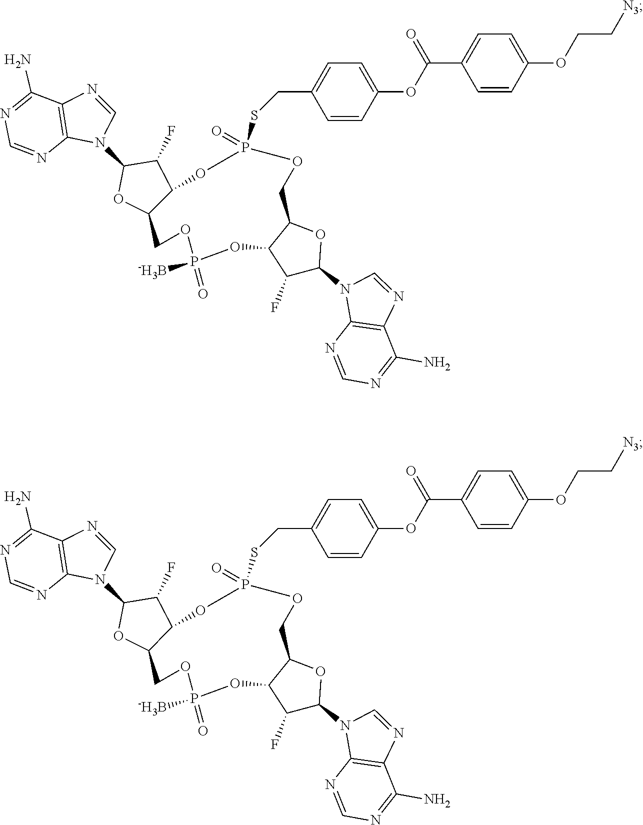 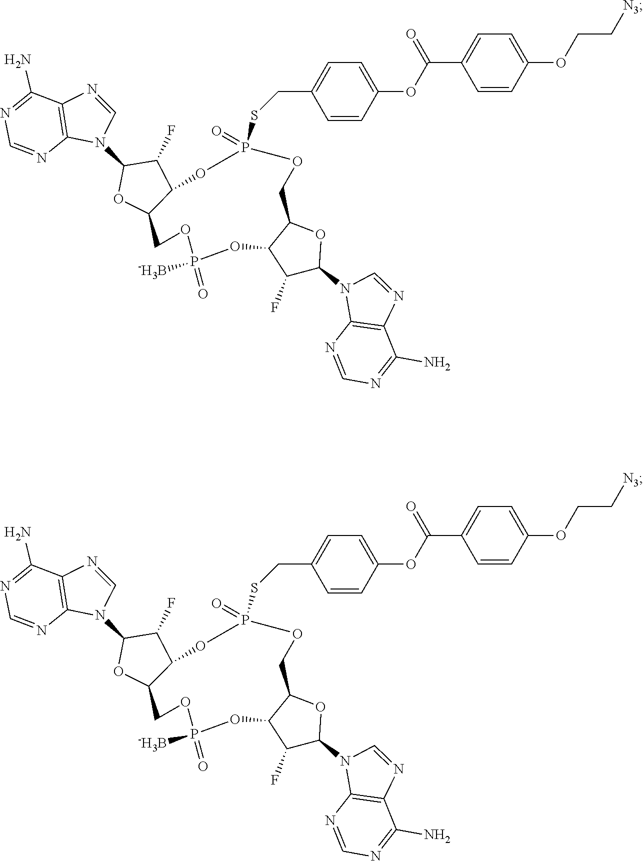 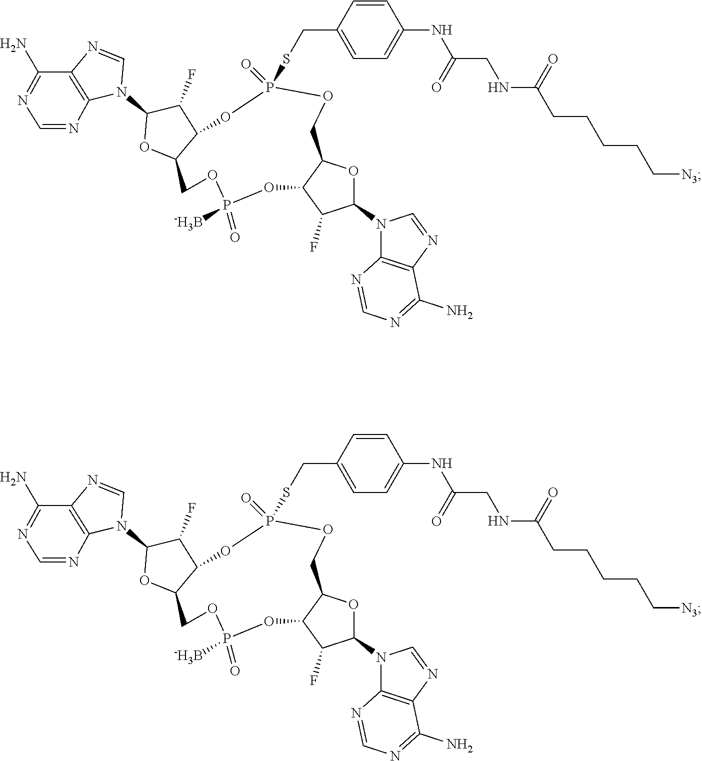 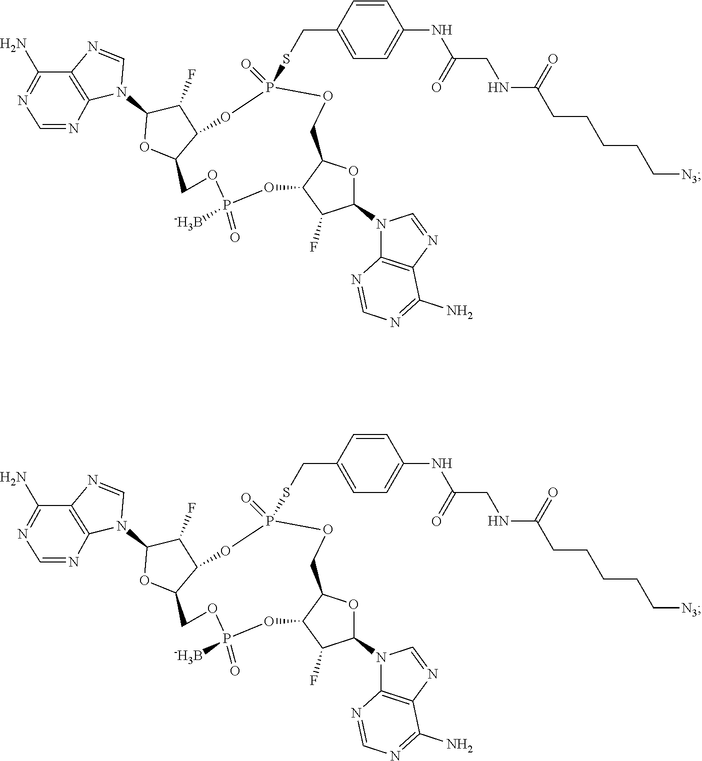 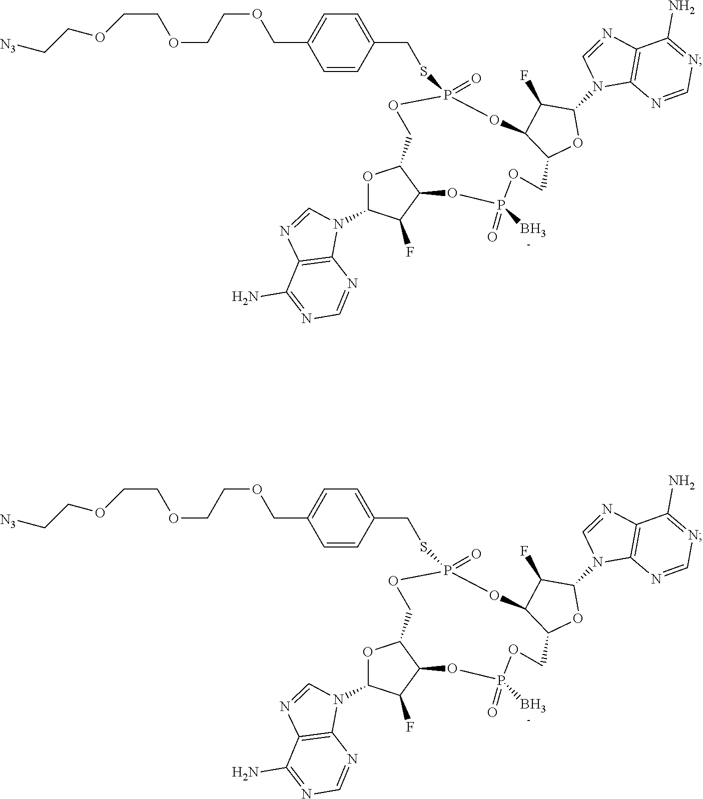 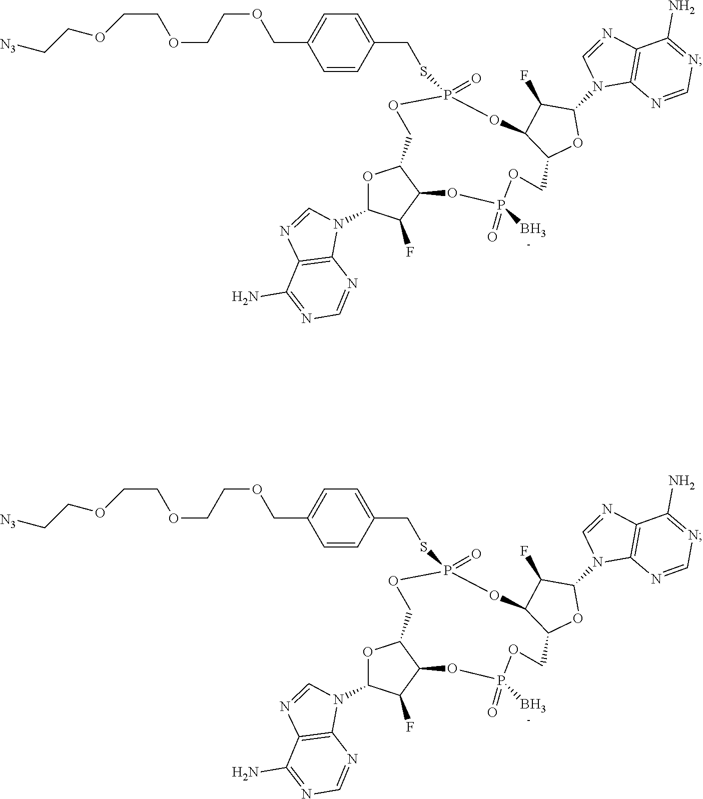 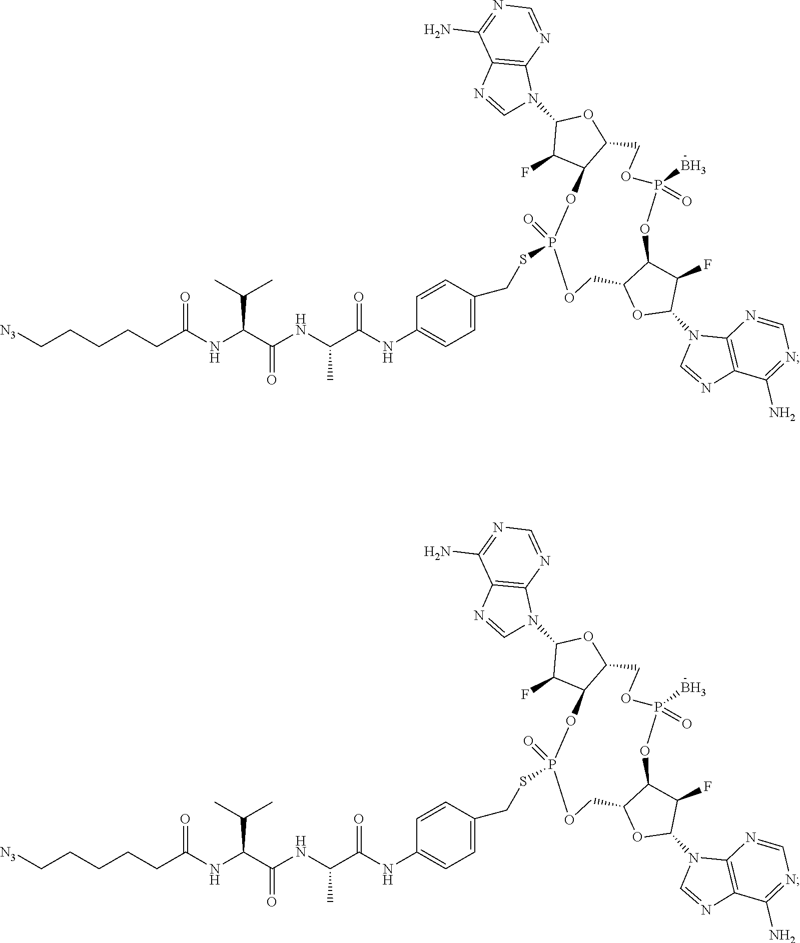 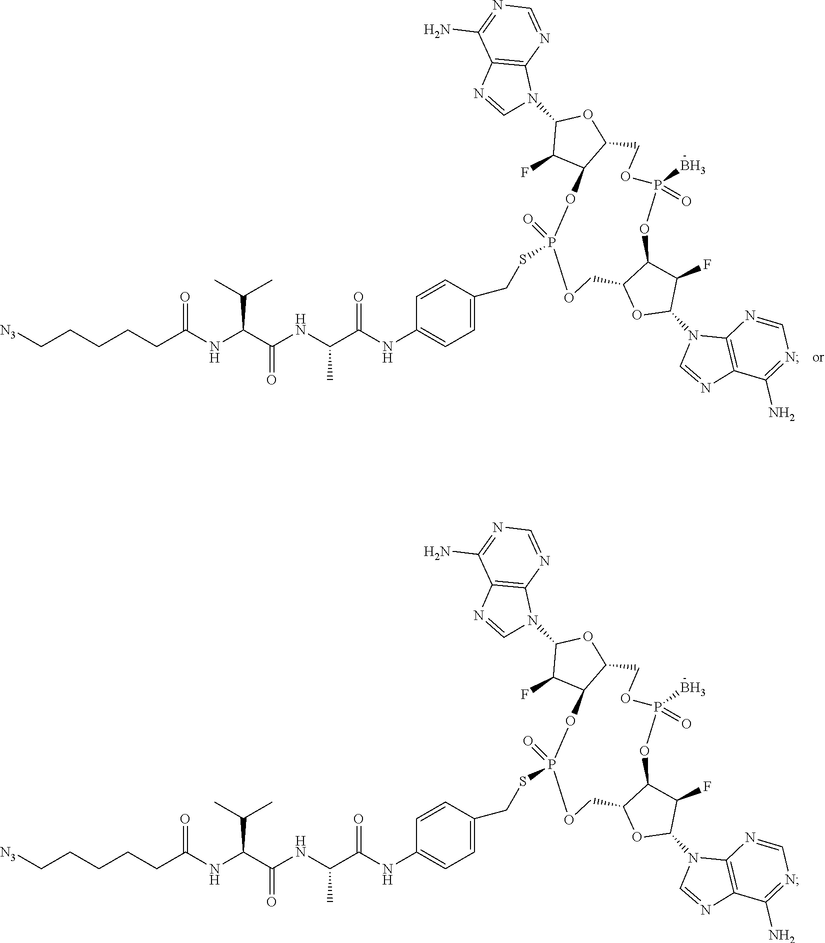 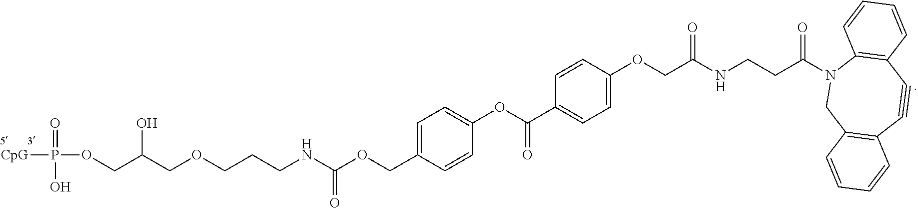
wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide; 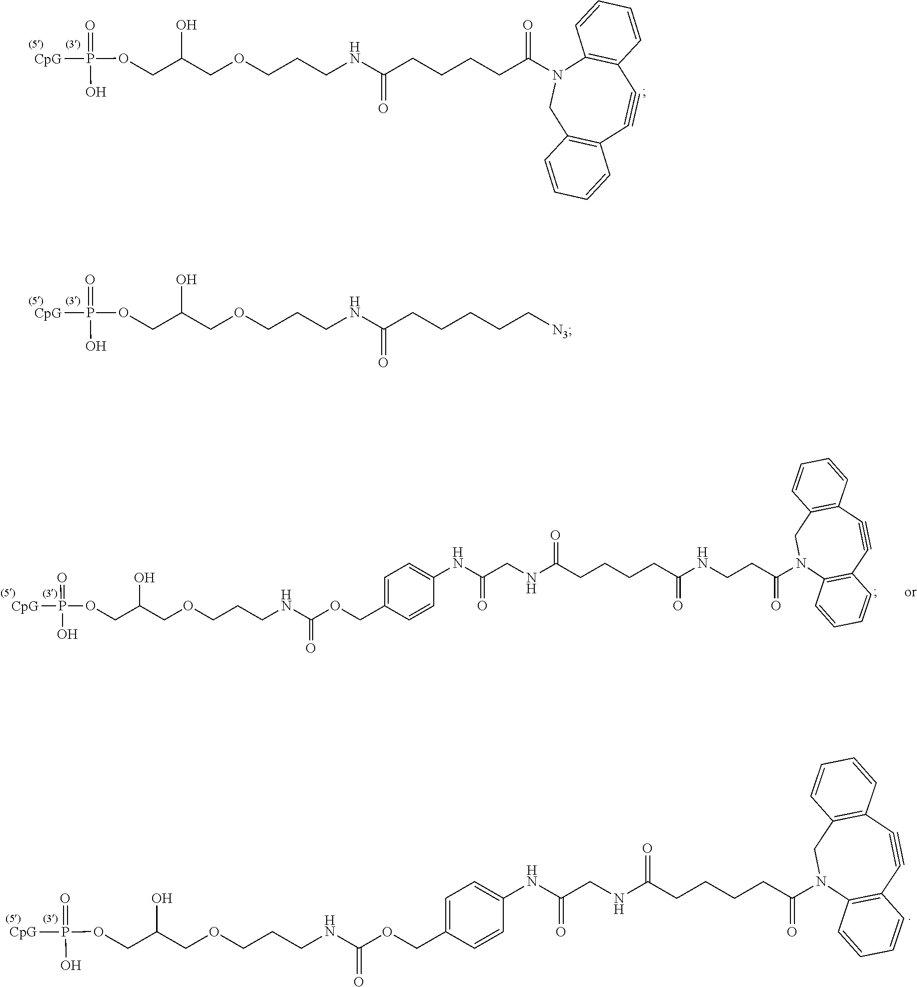
wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ (SD-101), connecting at 3′-O of the terminal nucleotide; 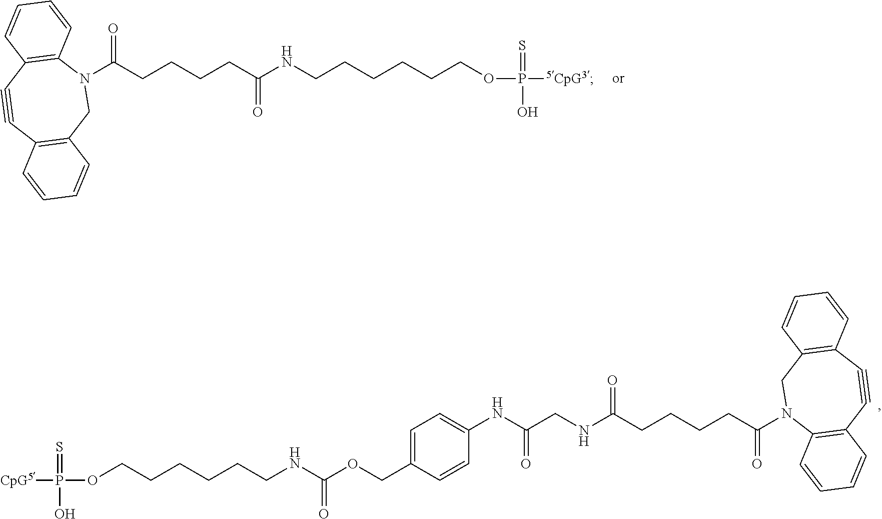
wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ (SD-101), connecting at 5′-O of the terminal nucleotide; 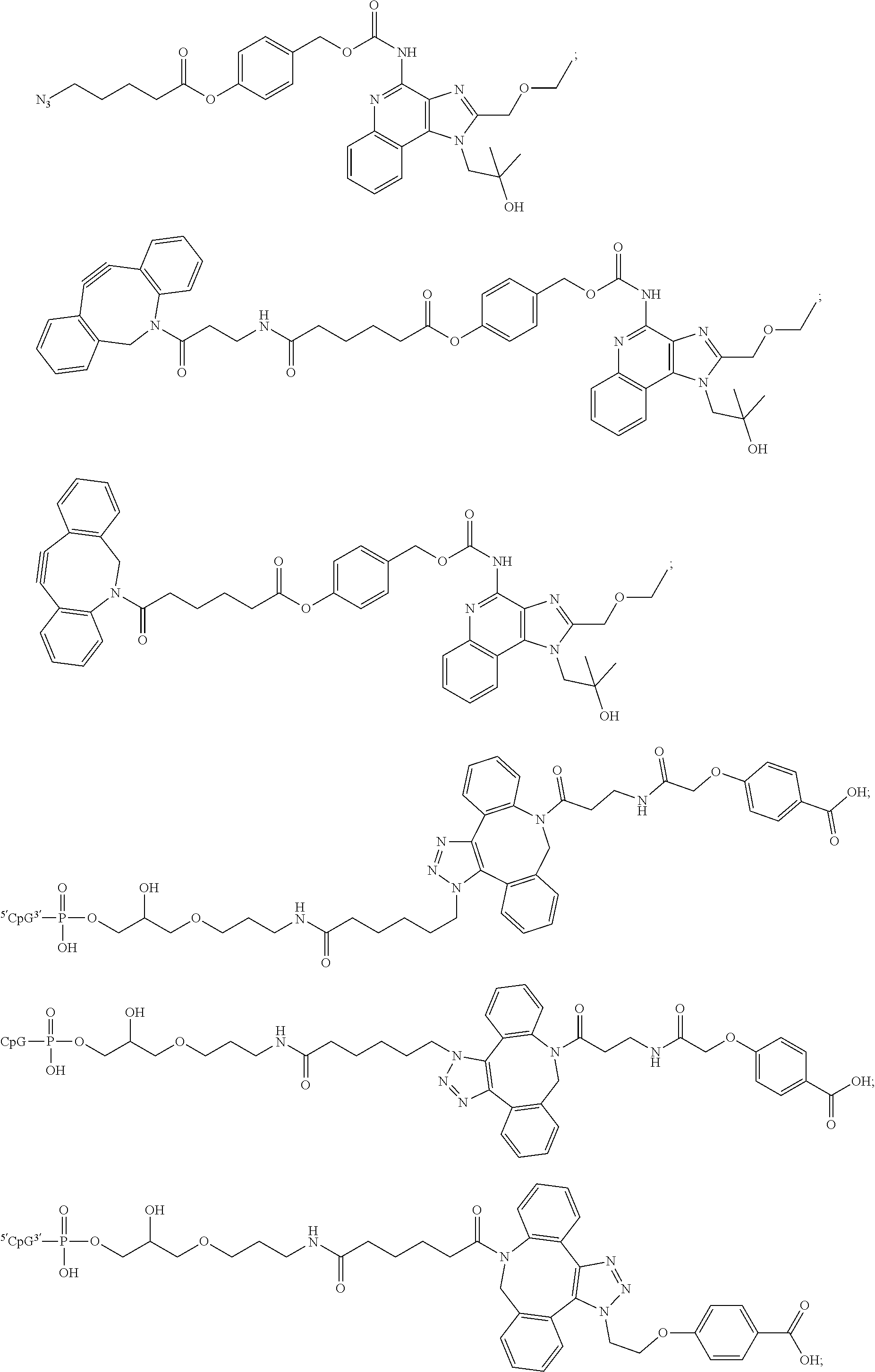 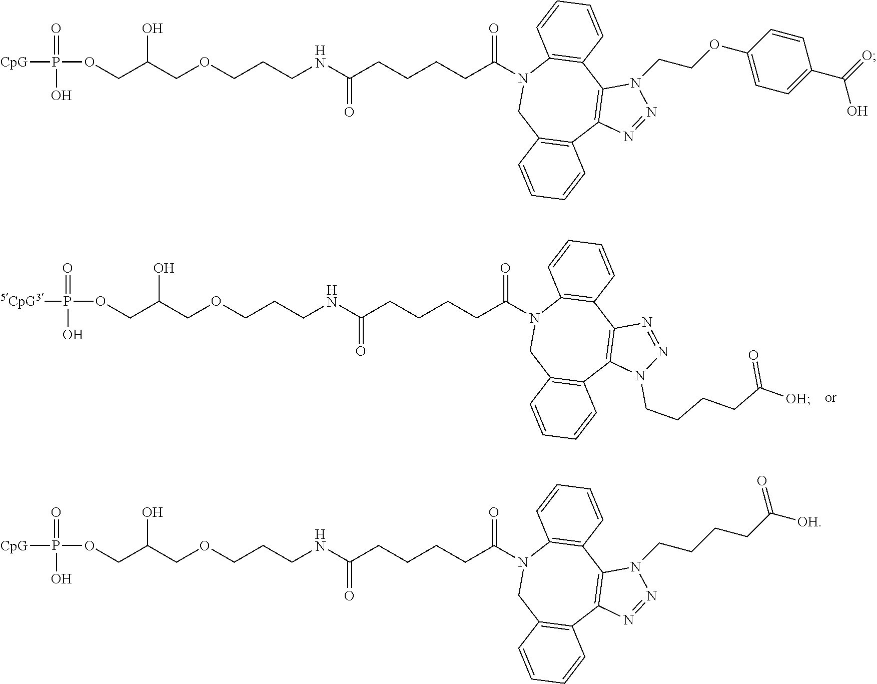
wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ connecting at 3′-O of the terminal nucleotide; 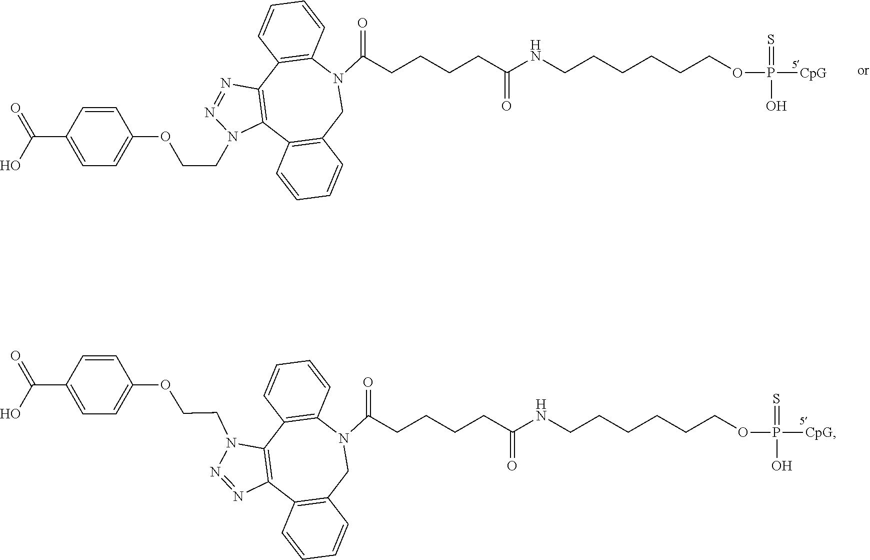
wherein CpG is a phosphorothioate linked oligodeoxynucleotide with a sequence of 5′-T*C*G *A*A*C *G*T*T *C*G*A *A*C*G *T*T*C *G*A*A *C*G*T *T*C*G *A*A*T-3′ connecting at 5′-O of the terminal nucleotide; 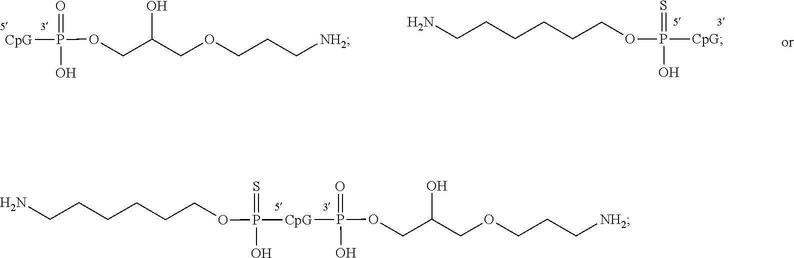
wherein CpG is a phosphorothioate linked oligodeoxynucleotides with a sequence of 5′-T*C*G *A*A*C *G*T*T *C*G*A *A*C*G *T*T*C *G*A*A *C*G*T *T*C*G *A*A*T-3′ (SD-101), connecting at either 5′-O or/and 3′-O of the terminal nucleotide.

122.-187. (canceled)

188. A linker having a structure of:
(i) a releasable linker of formula (II): 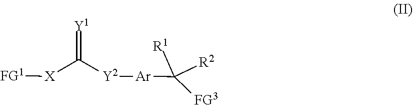
or a tautomer, a mixture of two or more tautomers, or an isotopic variant thereof;
wherein:
Ar is substituted or unsubstituted aryl, or substituted or unsubstituted heteroaryl;
X is a spacer moiety;
R¹ is hydrogen, substituted or unsubstituted alkyl, substituted or unsubstituted alkenyl, substituted or unsubstituted alkynyl, or substituted or unsubstituted aryl;
R² is hydrogen, substituted or unsubstituted alkyl, substituted or unsubstituted alkenyl, substituted or unsubstituted alkynyl, or substituted or unsubstituted aryl; or
R¹ and R² together with the atom to which they are attached, can join together to form a 3-8 membered ring that can contain one or two heteroatoms;
Y¹ is O, NH or S;
Y² is O, NH or S;
FG¹ is a functional group capable of reacting through click chemistry; and
FG³ is a functional group —OH, SH, LG¹ (leaving group 1) which includes but is not limited to —Cl, —Br, —I, 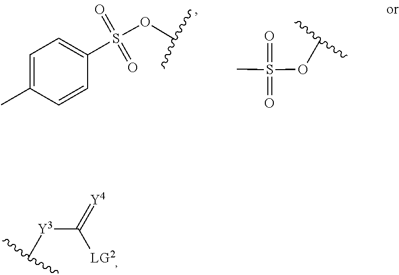
wherein Y³ is O or S; Y⁴ is O or S; LG 2 is a leaving group 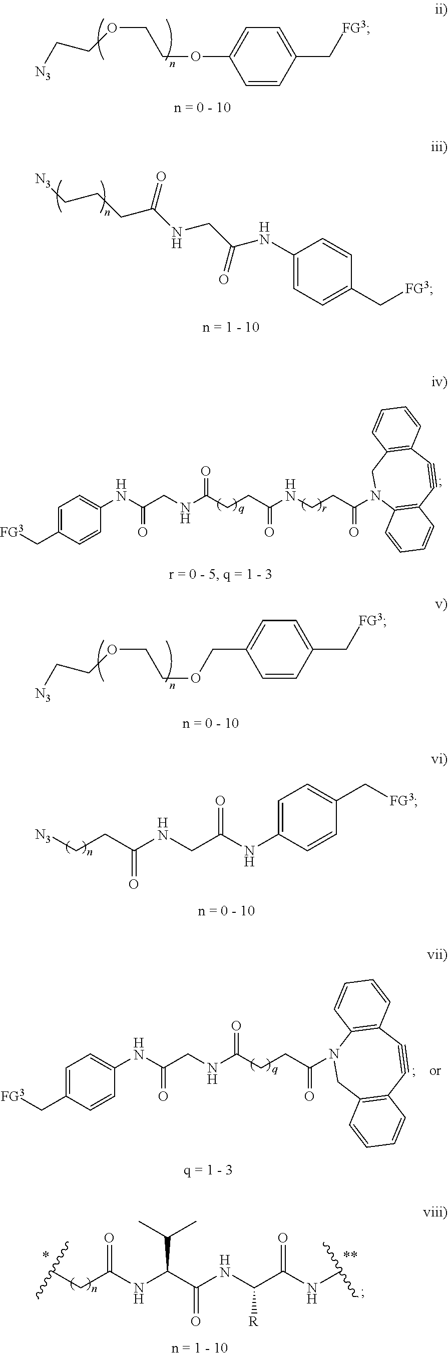
wherein R is methyl or 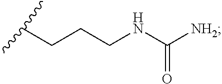 [SMILES: *CCCNC(N)=O]
wherein FG³ is independently —OH, —Cl, —I, or

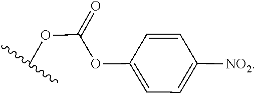 [SMILES: *OC(=O)Oc1ccc([N+](=O)[O-])cc1]

189.-192. (canceled)

193. The linker of claim 188, wherein the releasable linker of formula (II) has a structure of formula (II-C): 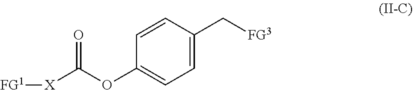
wherein:
X is: 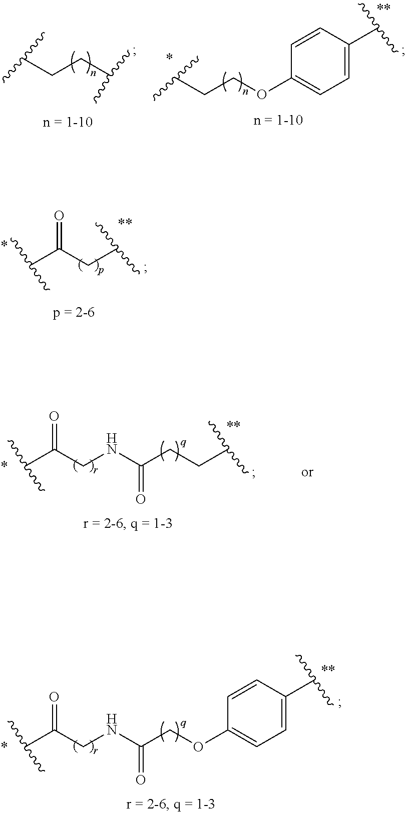
wherein * indicates the attachment point connecting to selected FG¹, ** indicates the attachment point connecting to the carbonyl group, and the spacer without * or ** indicates each of the two attachment points can be connected to either FG¹ or the carbonyl group; and
FG¹ is an azide, dibenzocyclooctyne (DBCO), or alkynyl.

194. (canceled)

195. The linker of claim 188, wherein the linker has the following structure:

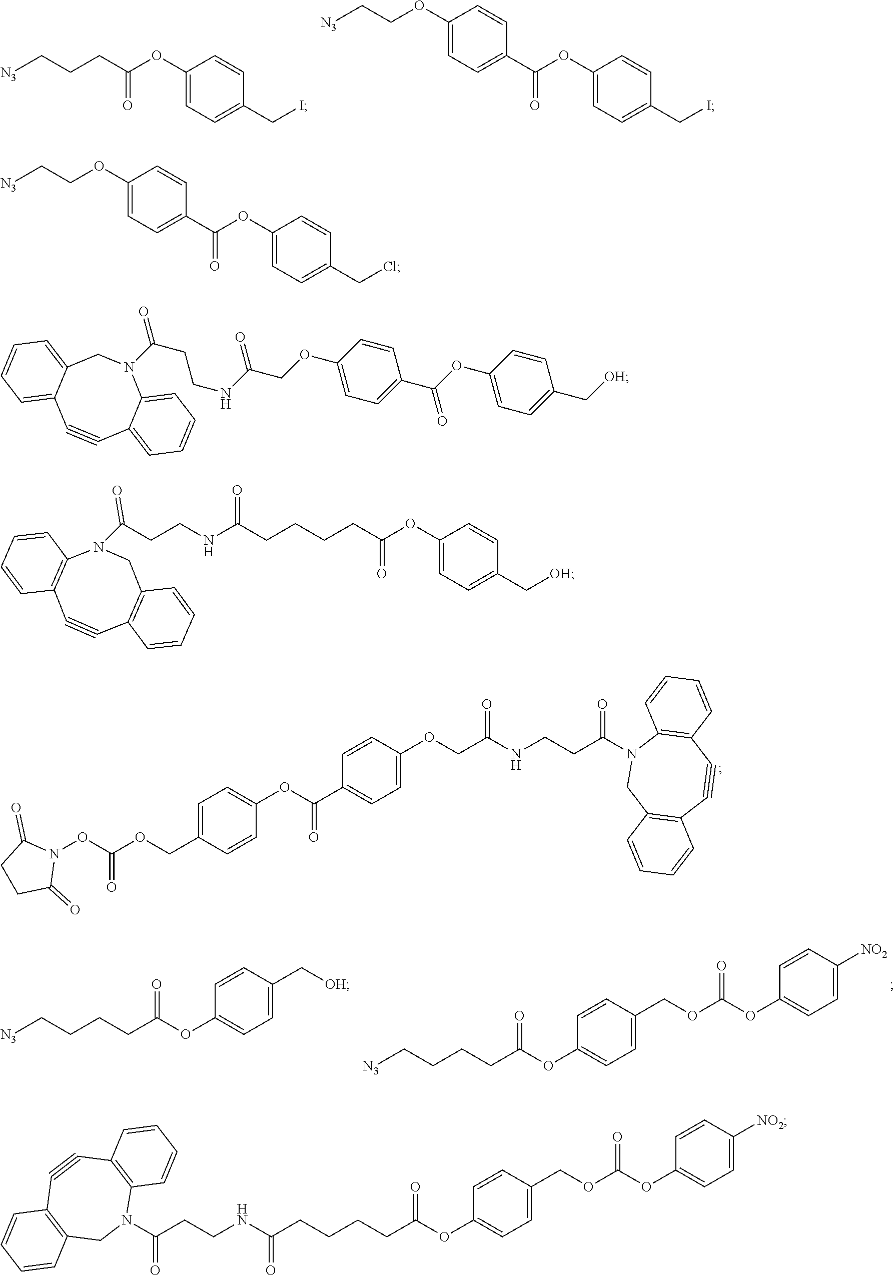

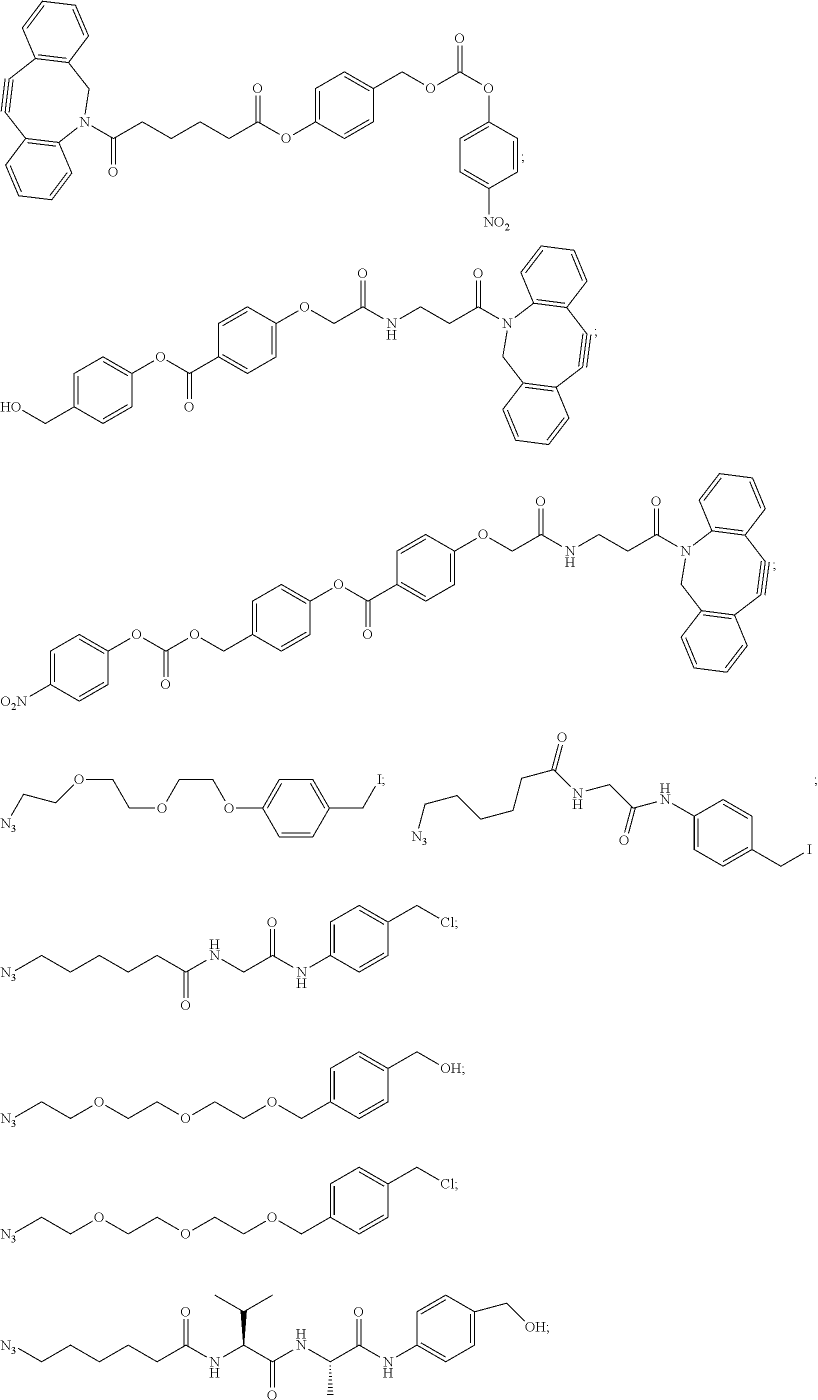

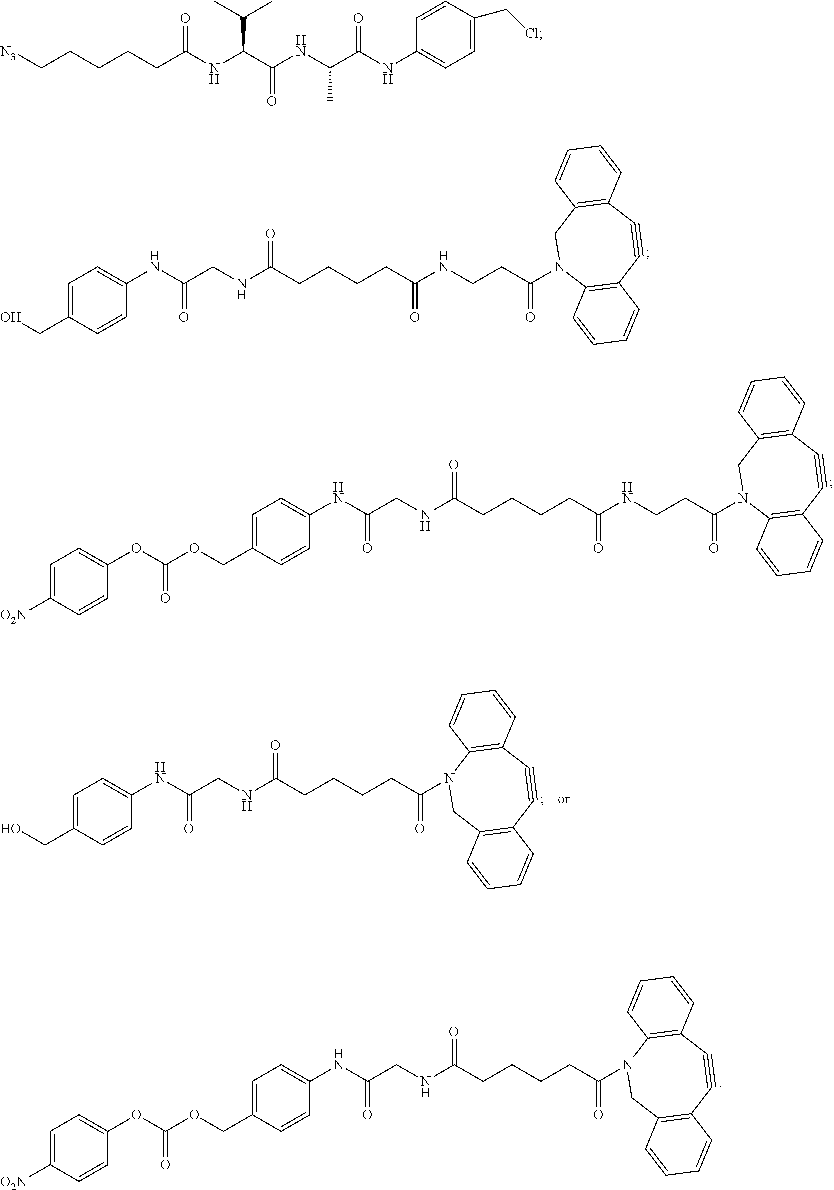 [SMILES: CC(C)[C@H](NC(=O)CCCCCN=[N+]=[N-])C(=O)N[C@@H](C)C(=O)Nc1ccc(CCl)cc1 ; O=C(CCCCC(=O)NCC(=O)Nc1ccc(CO)cc1)NCCC(=O)N1Cc2ccccc2C#Cc2ccccc21 ; O=C(CCCCC(=O)NCC(=O)Nc1ccc(COC(=O)Oc2ccc([N+](=O)[O-])cc2)cc1)NCCC(=O)N1Cc2ccccc2C#Cc2ccccc21 ; O=C(CCCCC(=O)N1Cc2ccccc2C#Cc2ccccc21)NCC(=O)Nc1ccc(CO)cc1 ; O=C(CCCCC(=O)N1Cc2ccccc2C#Cc2ccccc21)NCC(=O)Nc1ccc(COC(=O)Oc2ccc([N+](=O)[O-])cc2)cc1]

196.-210. (canceled)

211. A pharmaceutical composition comprising the conjugate of claim 1 and one or more pharmaceutically acceptable excipients.

212. A method of treating a disorder or disease in a subject in need thereof, comprising administering to the subject the pharmaceutical composition of claim 211.

213. (canceled)

214. A method of treating a disorder or disease in a subject in need thereof, comprising administering to the subject the pharmaceutical composition of claim 211 in combination with one or more additional therapeutic agents.

215.-220. (canceled)


SEQUENCE LISTING (4 sequences)

SEQ ID NO: 1 - DNA, 30 bp, Artificial Sequence
  TLR9 agonist
  misc_feature (1)..(30): joined by phosphorothioate bonds
tcgaacgttcgaacgttcgaacgttcgaat

SEQ ID NO: 2 - DNA, 22 bp, Artificial Sequence
  TLR9 agonist
  misc_feature (1)..(22): joined by phosphorothioate bonds
  modified_base (3)..(3): 2′-deoxy-7-deazaguanosine
  modified_base (7)..(7): 2′-deoxy-7-deazaguanosine
  modified_base (11)..(11): 2′-deoxy-7-deazaguanosine
  misc_feature (11)..(12): glycerol linker
  modified_base (12)..(12): 2′-deoxy-7-deazaguanosine
  modified_base (16)..(16): 2′-deoxy-7-deazaguanosine
  modified_base (20)..(20): 2′-deoxy-7-deazaguanosine
tcnaacnttcnncttncaanct

SEQ ID NO: 3 - DNA, 30 bp, Artificial Sequence
  TLR9 agonist
gggggggggggacgatcgtcgggggggggg

SEQ ID NO: 4 - DNA, 24 bp, Artificial Sequence
  TLR9 agonist
  misc_feature (1)..(24): joined by phosphorothioate bonds
tcgtcgttttgtcgttttgtcgtt